(19) United States
(12) Patent Application Publication
(10) Pub. No.: US 2026/0176235 A1
(43) Pub. Date: Jun. 25, 2026

(54) MONOMER, POLYMER, CHEMICALLY AMPLIFIED RESIST COMPOSITION, AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 19/422,761

(22) Filed: Dec. 17, 2025

(30) Foreign Application Priority Data
Dec. 24, 2024 (JP) 2024-227503

(51) Int. Cl. C07D 207/333; C07D 207/337; C07D 211/68; C08F 212/12; C08F 212/14; C08F 212/32; C08F 220/18; C08F 220/22; G03F 7/00; G03F 7/004

(52) U.S. Cl. CPC C07D 207/333; C07D 207/337; C07D 211/68; C08F 212/12; C08F 212/24; C08F 212/32; C08F 220/1806; C08F 220/22; G03F 7/0045; G03F 7/70033

(57) ABSTRACT

The polymer obtained from a monomer having a monocyclic lactam structure has excellent solvent solubility. The chemically amplified resist composition comprises the polymer. The chemically amplified resist composition exhibits a high sensitivity and contrast and forms a pattern of satisfactory profile having improved lithography properties such as EL, LWR, CDU and DOF.

### CROSS-REFERENCE TO RELATED APPLICATION

This non-provisional application claims priority under 35 U.S.C. § 119(a) on Patent Application No. 2024-227503 filed in Japan on Dec. 24, 2024, the entire contents of which are hereby incorporated by reference.

### TECHNICAL FIELD

This invention relates to a monomer, polymer, chemically amplified resist composition, and pattern forming process.

### BACKGROUND ART

To meet the demand for higher integration density and operating speed of LSIs, the effort to reduce the pattern rule is in rapid progress. As the use of 5G high-speed communications and artificial intelligence (AI) is widely spreading, high-performance devices are needed for their processing. As the advanced miniaturization technology, manufacturing of microelectronic devices at the 5-nm node by the lithography using EUV of wavelength 13.5 nm has been implemented in a mass scale. Studies are made on the application of EUV lithography to 3-nm node devices of the next generation and 2-nm node devices of the next-but-one generation.

As the feature size reduces, image blurs due to acid diffusion become a problem. To insure resolution for fine patterns of sub-45-nm size, not only an improvement in dissolution contrast is important as previously reported, but the control of acid diffusion is also important as reported in Non-Patent Document 1. Since chemically amplified resist compositions are designed such that sensitivity and contrast are enhanced by acid diffusion, an attempt to minimize acid diffusion by reducing the temperature and/or time of post-exposure bake (PEB) fails, resulting in drastic reductions of sensitivity and contrast.

A triangular tradeoff relationship among sensitivity, resolution, and edge roughness (LER or LWR) has been pointed out. Specifically, a resolution improvement requires to suppress acid diffusion whereas a short acid diffusion distance leads to a decline of sensitivity.

The addition of an acid generator capable of generating a bulky acid is an effective means for suppressing acid diffusion. It was then proposed to incorporate repeat units derived from an onium salt having a polymerizable unsaturated bond in a polymer. Since this polymer functions as an acid generator, it is referred to as polymer-bound acid generator. Patent Document 1 discloses a sulfonium or iodonium salt having a polymerizable unsaturated bond, capable of generating a specific sulfonic acid. Patent Document 2 discloses a sulfonium salt having a sulfonic acid directly attached to the backbone.

The component that contributes to the performance of a positive resist composition is required to achieve a high sensitivity and resolution and to form a resist pattern having tight adhesion to the substrate. As the unit for providing substrate adhesion, especially in the case of ArF lithography, methacrylates of monocyclic lactone such as butyrolactone or valerolactone ring and methacrylates of fused ring lactones such as norbornane lactone are known from Patent Documents 3 to 5. In Patent Documents 6 and 7, sultone units having a cyclic sulfonic acid ester structure are used for the same purpose as the lactone units. In Patent Document 8, N-methacryloxy-β-lactam is used for the same purpose as the lactone units. Also known are fused ring lactam units as typified by methacrylates of norbornane lactam (Patent Documents 9 and 10), methacrylates having succinimide structure (Patent Document 11), and methacrylates having a urea bond or thiourea bond (Patent Documents 12 and 13). In some examples, these units for providing substrate adhesion as applied to the ArF lithography are diverted to the EUV lithography, but they fail to display satisfactory performance from the aspects of pattern roughness and substrate adhesion.

To solve the outstanding problems and to meet the demand for further miniaturization, it is desired to develop base polymer units capable of achieving satisfactory resist performance in terms of sensitivity, resolution, acid diffusion length control, pattern roughness, and substrate adhesion.

### CITATION LIST

Patent Document 1: JP-A 2006-045311 (U.S. Pat. No. 7,482,108)
Patent Document 2: JP-A 2006-178317
Patent Document 3: JP 3042618
Patent Document 4: JP 4131062
Patent Document 5: JP-A 2006-146143
Patent Document 6: JP 5496715
Patent Document 7: JP 6909909
Patent Document 8: JP 5635514
Patent Document 9: JP 5478161
Patent Document 10: JP 5657443
Patent Document 11: JP 6642345
Patent Document 12: JP 5008838
Patent Document 13: JP 5576411
Non-Patent Document 1: SPIE Vol. 6520 65203L-1 (2007)

### SUMMARY OF THE INVENTION

An object of the invention is to provide a monomer, a polymer obtained therefrom, and a chemically amplified resist composition comprising the polymer, the resist composition having a high solvent solubility, high sensitivity and high contrast, and forming a resist pattern of satisfactory profile with improved lithography properties such as LWR, CDU, EL, and DOF when processed by photolithography using high-energy radiation such as KrF or ArF excimer laser, EB or EUV; and a pattern forming process using the resist composition.

The inventor has found that a polymer obtained from a monomer having a monocyclic lactam structure has excellent solvent solubility, and that a chemically amplified resist composition comprising the polymer exhibits a high sensitivity and contrast, is effective for reducing acid diffusion, and forms a resist pattern of satisfactory profile having improved lithography properties such as LWR, CDU, EL and DOF as well as substrate adhesion and being resistant to collapse in forming small-size patterns.

In one aspect, the invention provides a monomer having the formula (A).

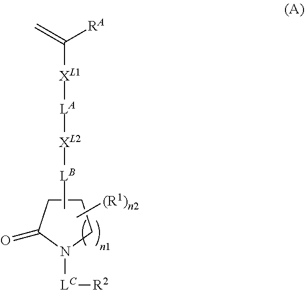

Herein n1 is 1, 2 or 3, n2 is 0, 1 or 2,
R^{A} is hydrogen, fluorine, methyl or trifluoromethyl,
X^{L1} is a single bond, optionally substituted phenylene group or optionally substituted naphthylene group,
X^{L2} is a single bond or C₁-C₄₀ hydrocarbylene group which may contain a heteroatom,
L^{A} and L^{B} are each independently a single bond, ether bond, ester bond, amide bond, carbonate bond or carbamate bond,
L^{C} is a single bond, carbonyl group or ester bond, R¹ is halogen, nitro, cyano, hydroxy, carboxy, or a C₁-C₂₀ hydrocarbyl group, C₁-C₂₀ hydrocarbyloxy group or C₁-C₂₀ hydrocarbylthio group, which may contain a heteroatom; when n2=2, two R¹ may be identical or different, and
R² is a C₁-C₂₀ hydrocarbyl group which may contain a heteroatom.

The preferred monomer has the formula (A1):

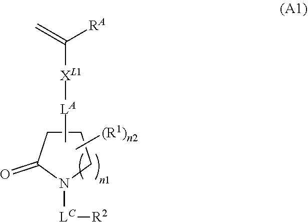

wherein n1, n2, R^{A}, X^{L1}, L^{A}, L^{C}, R¹ and R² are as defined above.

In another aspect, the invention provides a polymer comprising repeat units derived from the monomer defined herein.

The polymer may further comprise repeat units having the formula (a1) or (a2).

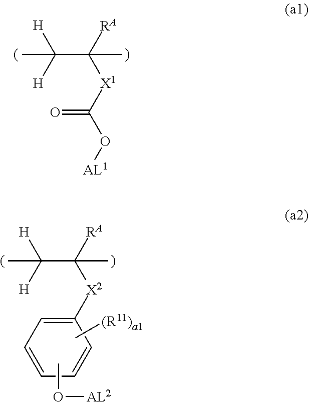

Herein R^{A} is each independently hydrogen, fluorine, methyl or trifluoromethyl,
X¹ is a single bond, phenylene, naphthylene, *—C(═O)—O—X¹¹— or *—C(═O)—NH—X¹¹—, the phenylene or naphthylene group may be substituted with hydroxy, nitro, cyano, optionally fluorinated C₁-C₁₀ saturated hydrocarbyl moiety, optionally fluorinated C₁-C₁₀ saturated hydrocarbyloxy moiety, or halogen, X¹¹ is a C₁-C₁₀ saturated hydrocarbylene group, phenylene group or naphthylene group, the saturated hydrocarbylene group may contain hydroxy, ether bond, ester bond or lactone ring,
X² is a single bond, *—C(═O)—O— or *—C(═O)—NH—,
* designates a point of attachment to the carbon atom in the backbone,
R¹¹ is halogen, cyano, hydroxy, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom; when a1 is 2, 3 or 4, a plurality of R¹¹ may be identical or different,
AL¹ and AL² are each independently an acid labile group, and
a1 is 0, 1, 2, 3 or 4.

The polymer may further comprise repeat units having the formula (a3).

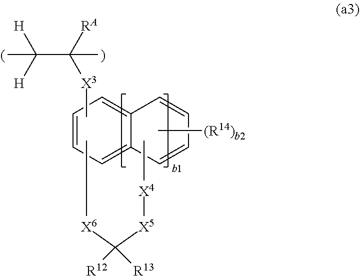

Herein b1 is 0 or 1, b2 is 0, 1, 2 or 3 when b1=0, b2 is 0, 1, 2, 3, 4 or 5 when b1=1,
R^{A} is hydrogen, fluorine, methyl or trifluoromethyl,
X³ is a single bond, *—C(═O)—O— or *—C(═O)—NH—, * designates a point of attachment to the carbon atom in the backbone,
X⁴ is a single bond, C₁-C₄ aliphatic hydrocarbylene group, carbonyl, sulfonyl or a group obtained by combining the foregoing,
X⁵ and X⁶ are each independently oxygen or sulfur, X⁴ and X⁶ are attached to adjoining carbon atoms on the aromatic ring,
R¹² and R¹³ are each independently hydrogen or a C₁-C₂₀ hydrocarbyl group which may contain a heteroatom, R¹² and R¹³ may bond together to form a ring with the carbon atom to which they are attached,
R¹⁴ is halogen, hydroxy, cyano, nitro, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{14A})(R^{14B}), R^{14A} and R^{14B} are each independently hydrogen or a C₁-C₆ hydrocarbyl group; and when b2 is 2 or more, a plurality of R¹⁴ may be identical or different and a plurality of R¹⁴ may bond together to form a ring with the carbon atom on the aromatic ring to which they are attached.

The polymer may further comprise repeat units having the formula (b1) or (b2).

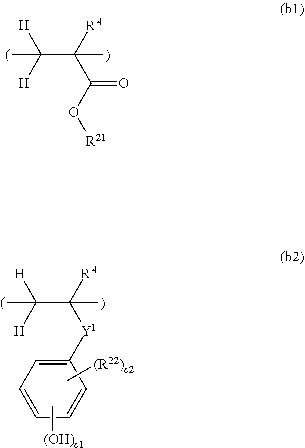

Herein R^{A} is hydrogen, fluorine, methyl or trifluoromethyl,
Y¹ is a single bond or *—C(═O)—O—, * designates a point of attachment to the carbon atom in the backbone,
R²¹ is hydrogen or a C₁-C₂₀ group containing at least one structure selected from hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—),
R²² is halogen, carboxy, nitro, cyano, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom; when c2 is 2, 3 or 4, a plurality of R²² may be identical or different,
c1 is 1, 2, 3 or 4, c2 is 0, 1, 2, 3 or 4, and 1≤c1+c2≤5.

The polymer may further comprise repeat units of at least one type selected from repeat units having the formula (c1), repeat units having the formula (c2), repeat units having the formula (c3), repeat units having the formula (c4), and repeat units having the formula (c5).

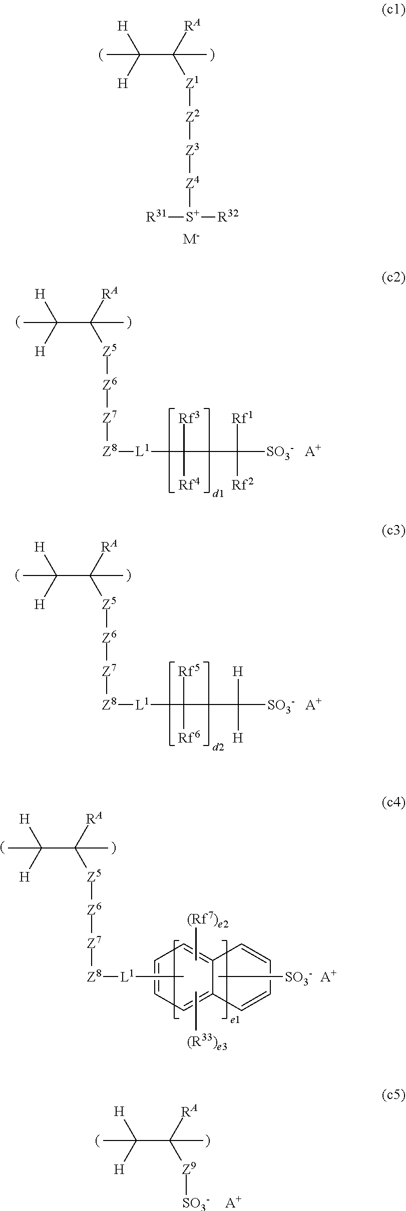

Herein d1 and d2 are each independently 0, 1, 2 or 3,
e1 is 0 or 1, e2 is 0, 1, 2, 3 or 4, e3 is 0, 1, 2, 3 or 4, meeting 0≤e2+e3≤4 when e1=0, and 0≤e2+e3≤6 when e1=1,
R^{A} is each independently hydrogen, fluorine, methyl or trifluoromethyl,
Z¹ is a single bond or optionally substituted phenylene group,
Z² is a single bond, **—C(═O)—O—Z²¹—, **—C(═O)—NH—Z²¹—, or **—O—Z²¹—, Z²¹ is a C₁-C₆ aliphatic hydrocarbylene group, phenylene group or a divalent group obtained by combining the foregoing, which may contain halogen, carbonyl moiety, ester bond, ether bond or hydroxy moiety,
Z³ is a single bond, ether bond, ester bond, sulfonate ester bond, amide bond, sulfonamide bond, carbonate bond or carbamate bond,
Z⁴ is a single bond or a C₁-C₆ aliphatic hydrocarbylene group, phenylene group or a divalent group obtained by combining the foregoing, which may contain halogen, carbonyl moiety, ester bond, ether bond or hydroxy moiety,
Z⁵ is each independently a single bond, optionally substituted phenylene group, naphthylene group or *—C(═O)—O—Z⁵¹—, Z⁵¹ is a C₁-C₁₀ aliphatic hydrocarbylene group, phenylene group or naphthylene group, the aliphatic hydrocarbylene group may contain halogen, hydroxy, ether bond, ester bond or lactone ring,
Z⁶ is a single bond, ether bond, ester bond, sulfonate ester bond, amide bond, sulfonamide bond, carbonate bond or carbamate bond,
Z⁷ is each independently a single bond, ***—Z⁷—C(═O)—O—, ***—C(═O)—NH—Z⁷— or ***—O—Z⁷—, Z⁷¹ is a C₁-C₂₀ hydrocarbylene group which may contain a heteroatom,
Z⁸ is each independently a single bond, ****—Z⁸—C(═O)—O—, ****—C(═O)—NH—Z⁸¹— or ****—O—Z⁸′—, Z⁸¹ is a C₁-C₂₀ hydrocarbylene group which may contain a heteroatom,
Z⁹ is a single bond, methylene, ethylene, phenylene, fluorinated phenylene, trifluoromethyl-substituted phenylene, *—C(═O)—O—Z⁹¹—, *—C(═O)—N(H)—Z⁹¹— or *—O—Z⁹¹—, Z⁹¹ is a C₁-C₆ aliphatic hydrocarbylene group, phenylene group, fluorinated phenylene group, or trifluoromethyl-substituted phenylene group, which may contain carbonyl, ester bond, ether bond or hydroxy,
* designates a point of attachment to the carbon atom in the backbone, ** designates a point of attachment to Z¹, *** designates a point of attachment to Z⁶, **** designates a point of attachment to Z⁷,
L¹ is a single bond, ether bond, ester bond, carbonyl group, sulfonate ester bond, sulfonamide bond, carbonate bond or carbamate bond,
Rf¹ and Rf² are each independently fluorine or a C₁-C₆ fluorinated saturated hydrocarbyl group,
Rf³ and Rf⁴ are each independently hydrogen, fluorine or a C₁-C₆ fluorinated saturated hydrocarbyl group,
Rf⁵ and Rf⁶ are each independently hydrogen, fluorine or a C₁-C₆ fluorinated saturated hydrocarbyl group, excluding that all Rf⁵ and Rf⁶ are hydrogen at the same time,
Rf⁷ is fluorine, a C₁-C₆ fluorinated alkyl group, C₁-C₆ fluorinated alkoxy group, or C₁-C₆ fluorinated alkylthio group; when e2 is 2, 3 or 4, a plurality of Rf may be identical or different,
R³¹ and R³² are each independently a C₁-C₂₀ hydrocarbyl group which may contain a heteroatom, R³¹ and R³² may bond together to form a ring with the sulfur atom to which they are attached,
R³³ is halogen exclusive of fluorine, or a C₁-C₂₀ hydrocarbyl group which may contain a heteroatom; when e3 is 2, 3 or 4, a plurality of R³³ may be identical or different and a plurality of R³³ may bond together to form a ring with the carbon atoms to which they are attached,
M⁻ is a non-nucleophilic counter ion, and
A⁺ is an onium cation.

In a further aspect, the invention provides a chemically amplified resist composition comprising a base polymer containing the polymer defined herein.

The resist composition may further comprise an organic solvent, quencher, acid generator, and/or surfactant.

In a still further aspect, the invention provides a pattern forming process comprising the steps of applying the chemically amplified resist composition defined herein onto a substrate to form a resist film thereon, exposing the resist film to high-energy radiation, and developing the exposed resist film in a developer.

Typically, the high-energy radiation is KrF excimer laser, ArF excimer laser, EB or EUV of wavelength 3 to 15 nm.

### Advantageous Effects of the Invention

When a chemically amplified resist composition comprising a polymer obtained from polymerization of the inventive monomer is processed by lithography, resist patterns having a high sensitivity, high contrast, and improved lithography properties including LWR, CDU, EL, and DOF can be formed. The risk of pattern collapse during formation of small-size patterns is minimized.

### DESCRIPTION OF THE PREFERRED EMBODIMENT

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. In chemical formulae, Me stands for methyl, and Ac for acetyl. The broken line (---) and the asterisk (*) designate a valence bond or point of attachment. As used herein, the term “halogenated” refers to a halogen-substituted or halogen-containing compound or group. The terms “group” and “moiety” are interchangeable.

The abbreviations and acronyms have the following meaning.
EB: electron beam
EUV: extreme ultraviolet
Mw: weight average molecular weight
Mn: number average molecular weight
Mw/Mn: molecular weight dispersity
GPC: gel permeation chromatography
PEB: post-exposure bake
PAG: photoacid generator
EL: exposure latitude
LWR: line width roughness
CDU: critical dimension uniformity
DOF: depth of focus

It is understood that for some structures represented by chemical formulae, there can exist enantiomers and diastereomers because of the presence of asymmetric carbon atoms. In such a case, a single formula collectively represents all such isomers. The isomers may be used alone or in admixture.

### [Monomer]

The invention provides a monomer having the formula (A).

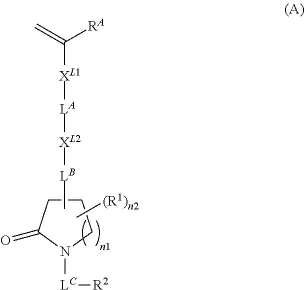

In formula (A), n1 is 1, 2 or 3. The relevant structure is a 5-membered ring when n1=1, a 6-membered ring when n1=2, and a 7-membered ring when n1=3. It is preferred from the aspect of synthesis that n1 be 1 or 2. The subscript n2 is 0, 1 or 2. It is preferred for reactant availability that n2 be 0 or 1.

In formula (A), R^{A} is hydrogen, fluorine, methyl or trifluoromethyl. Of these, hydrogen and methyl are preferred.

In formula (A), X^{L1} is a single bond, optionally substituted phenylene group or optionally substituted naphthylene group, preferably a single bond or optionally substituted phenylene group. Preferred substituents include halogen, cyano, hydroxy, nitro, pentafluorosulfanyl, C₁-C₂₀ hydrocarbyl groups which may contain a heteroatom, C₁-C₂₀ hydrocarbyloxy groups which may contain a heteroatom, C₂-C₂₀ hydrocarbylcarbonyl groups which may contain a heteroatom, C₂-C₂₀ hydrocarbylcarbonyloxy groups which may contain a heteroatom, and C₂-C₂₀ hydrocarbyloxycarbonyl groups which may contain a heteroatom.

In formula (A), X^{L2} is a single bond or C₁-C₄₀ hydrocarbylene group which may contain a heteroatom. The hydrocarbylene group may be straight, branched or cyclic. Examples include alkanediyl groups, cyclic saturated hydrocarbylene groups, and arylene groups. Suitable heteroatoms include oxygen, nitrogen and sulfur.

Examples of the C₁-C₄₀ hydrocarbylene group which may contain a heteroatom, represented by X^{L2}, are shown below, but not limited thereto. Herein * designates a point of attachment to L^{A} or L^{B}.

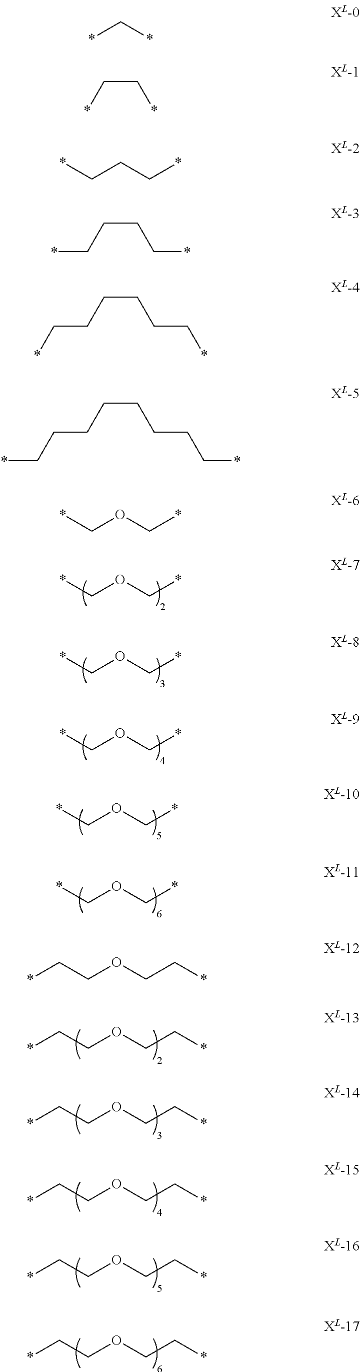

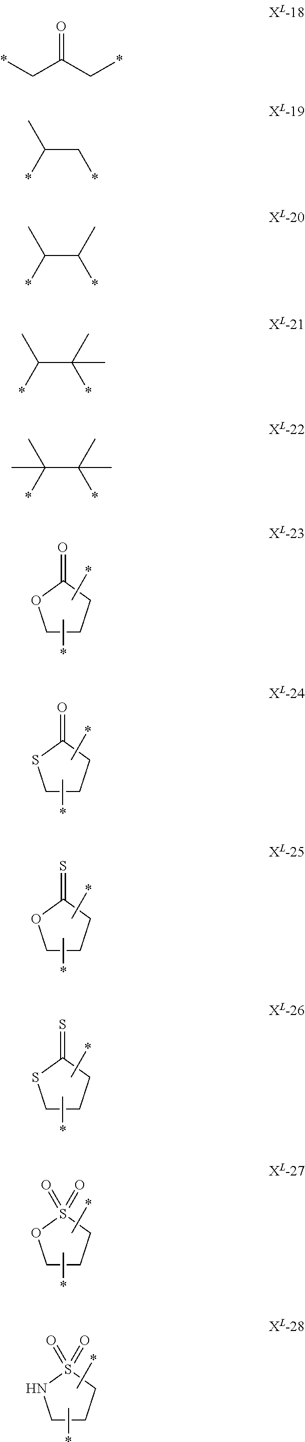

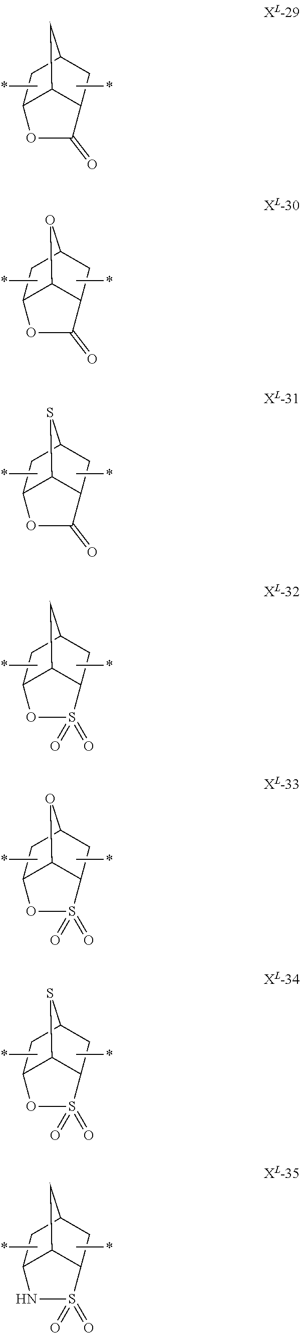

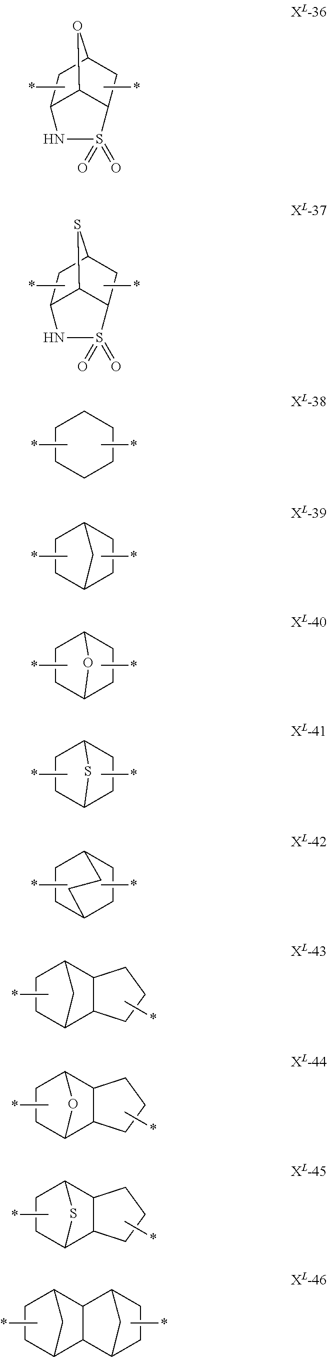

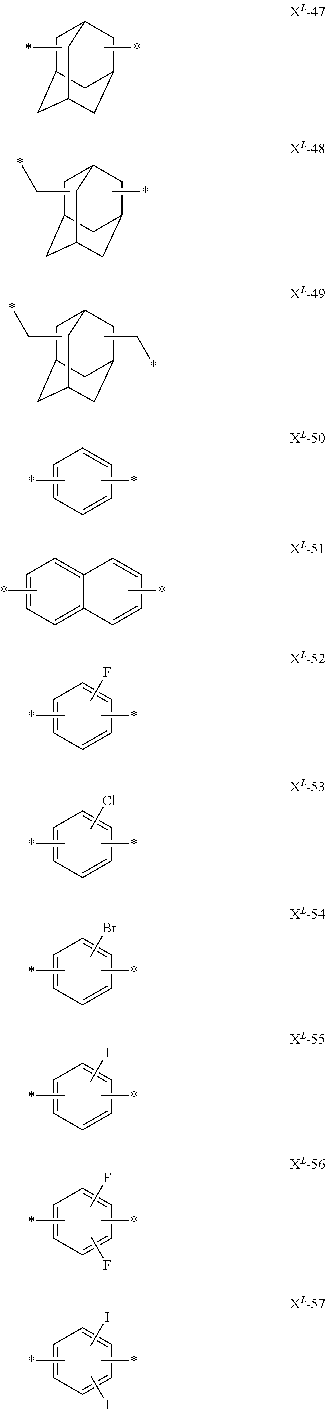

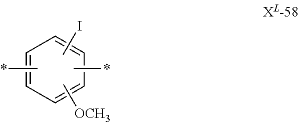

Of these, X^{L}-0 to X^{L}-22, X^{L}-29 to X^{L}-34, and X^{L}-47 to X^{L}-58 are preferred.

In formula (A), L^{A} and L^{B} are each independently a single bond, ether bond, ester bond, amide bond, carbonate bond or carbamate bond. L^{A} is preferably an ester bond or amide bond, more preferably an ester bond, when X^{L1} is a single bond. L^{A} is preferably an ether, ester, amide, carbonate or carbamate bond, more preferably an ether or ester bond, when X^{L1} is an optionally substituted phenylene or naphthylene group. L^{B} is preferably an ester or amide bond when X^{L2} is a C₁-C₄₀ hydrocarbylene group which may contain a heteroatom. L^{B} is preferably a single bond when X^{L2} is a single bond.

In formula (A), L^{C} is a single bond, carbonyl group or ester bond.

In formula (A), R¹ is halogen, nitro, cyano, hydroxy, carboxy, or a C₁-C₂₀ hydrocarbyl group, C₁-C₂₀ hydrocarbyloxy group or C₁-C₂₀ hydrocarbylthio group. The hydrocarbyl group, hydrocarbyloxy group or hydrocarbylthio group may contain a heteroatom. Suitable halogen atoms include fluorine, chlorine, bromine, and iodine, with fluorine and iodine being preferred. The hydrocarbyl group may be saturated or unsaturated and straight, branched or cyclic. Examples thereof include C₁-C₂₀ alkyl groups such as methyl, ethyl, n-propyl, isopropyl, n-butyl, and tert-butyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, allyl, propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl, and combinations thereof. Inter alia, aryl groups are preferred. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety. When n2 is 2, two R¹ may be identical or different.

In formula (A), R² is a C₁-C₂₀ hydrocarbyl group which may contain a heteroatom. The hydrocarbyl group may be saturated or unsaturated and straight, branched or cyclic. Examples thereof are as exemplified above for the hydrocarbyl group R¹, but not limited thereto. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety.

The monomer of formula (A) preferably has the formula (A1).

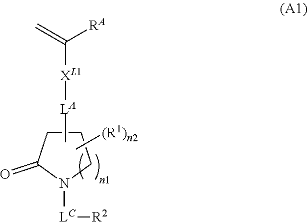

Herein n1, n2, R^{A}, X^{L1}, L^{A}, L^{C}, R¹ and R² are as defined above.

Examples of the monomer having formula (A) are shown below, but not limited thereto. Herein R^{A} is as defined above.

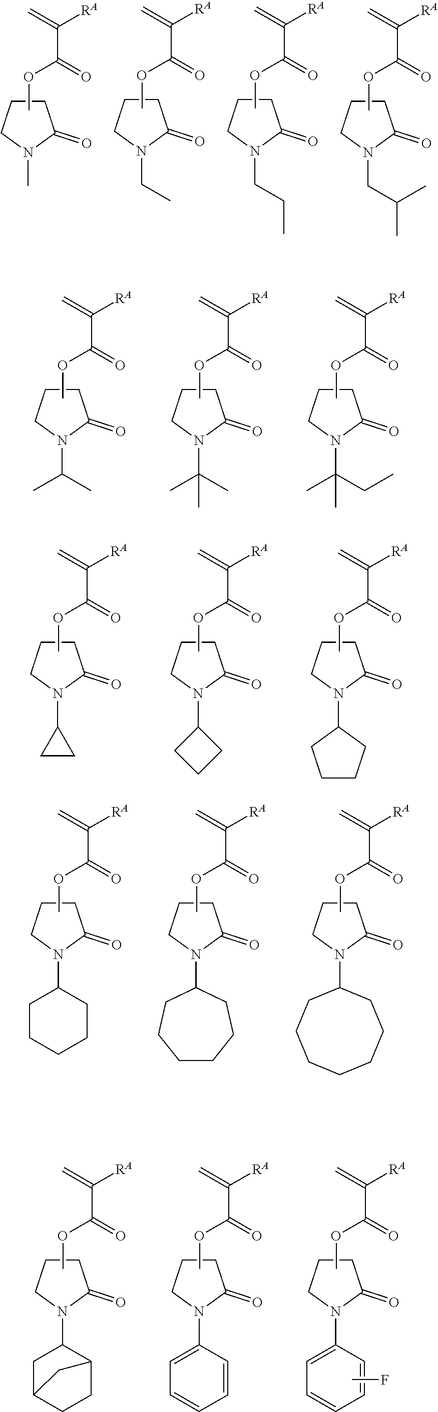

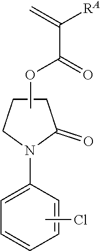

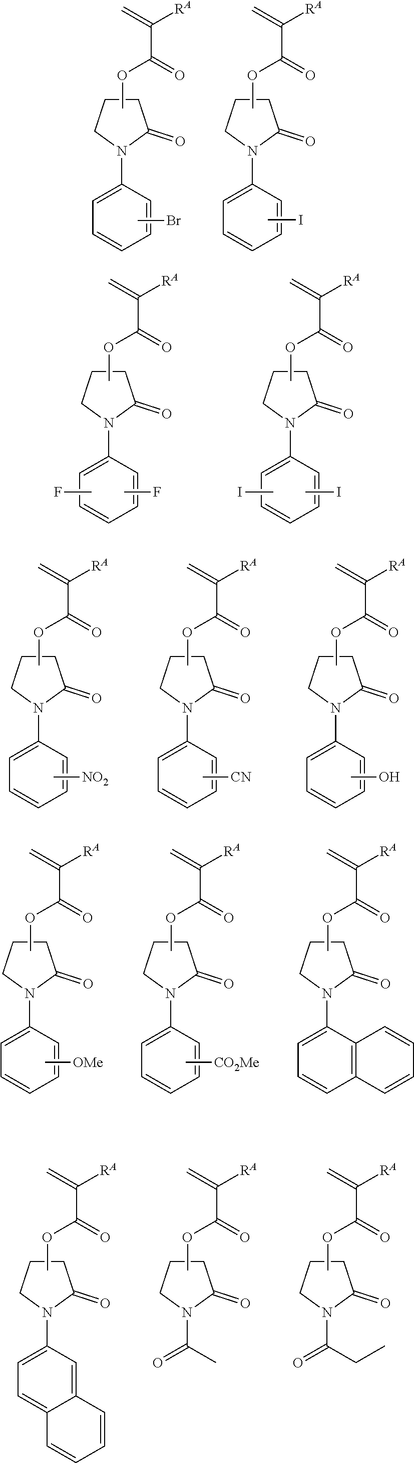

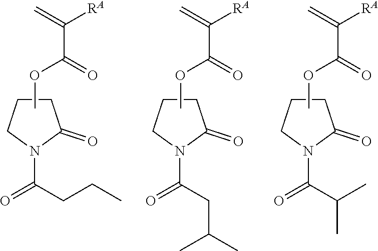

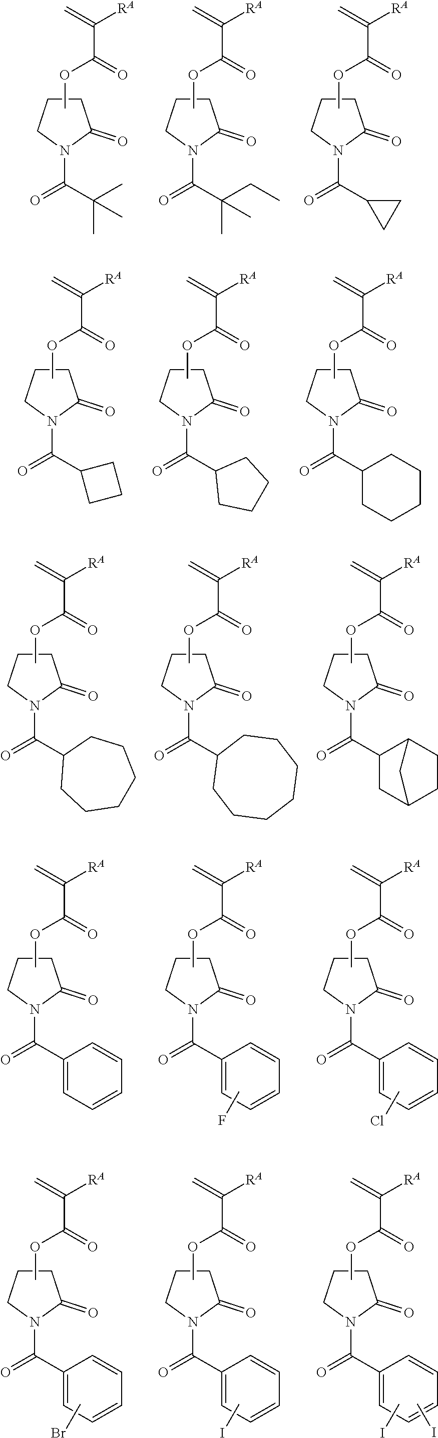

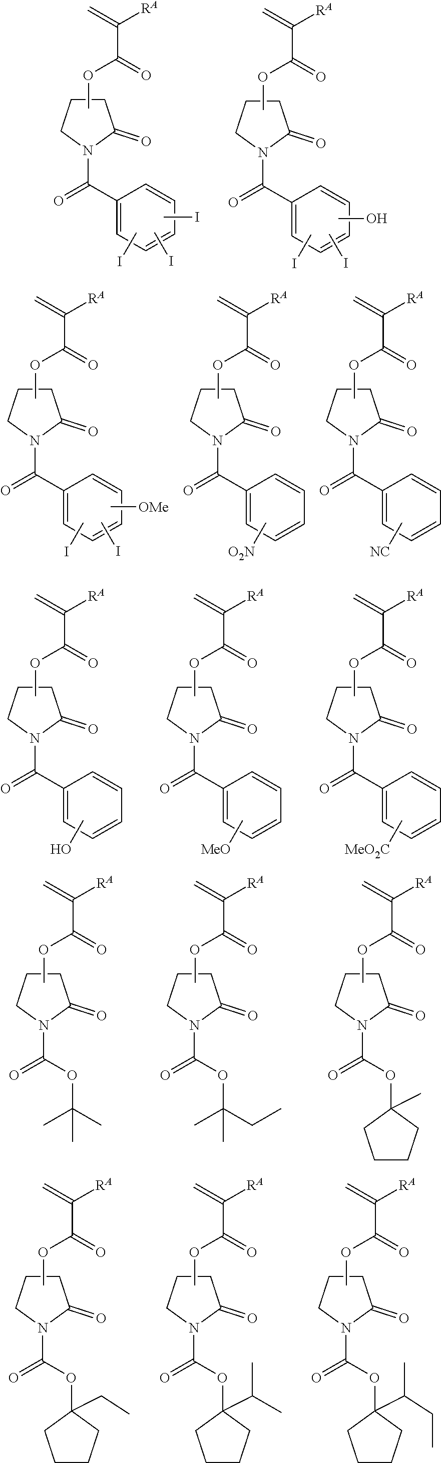

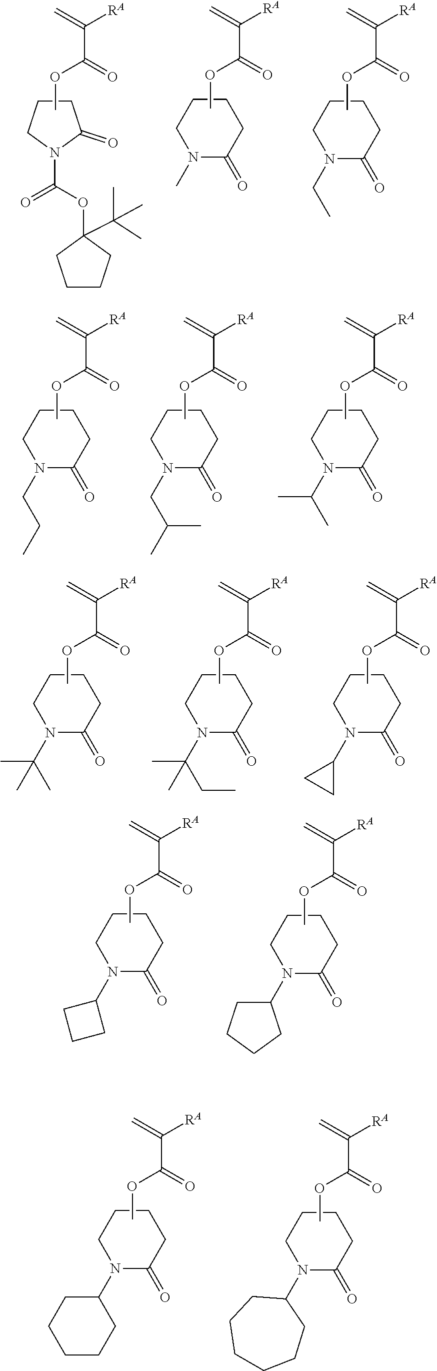

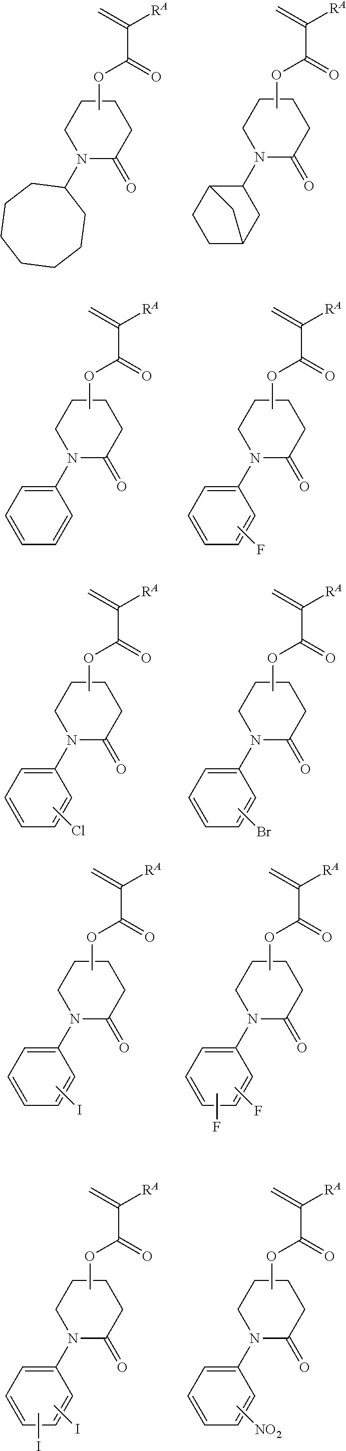

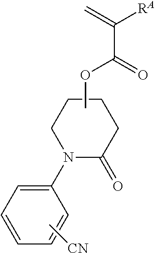

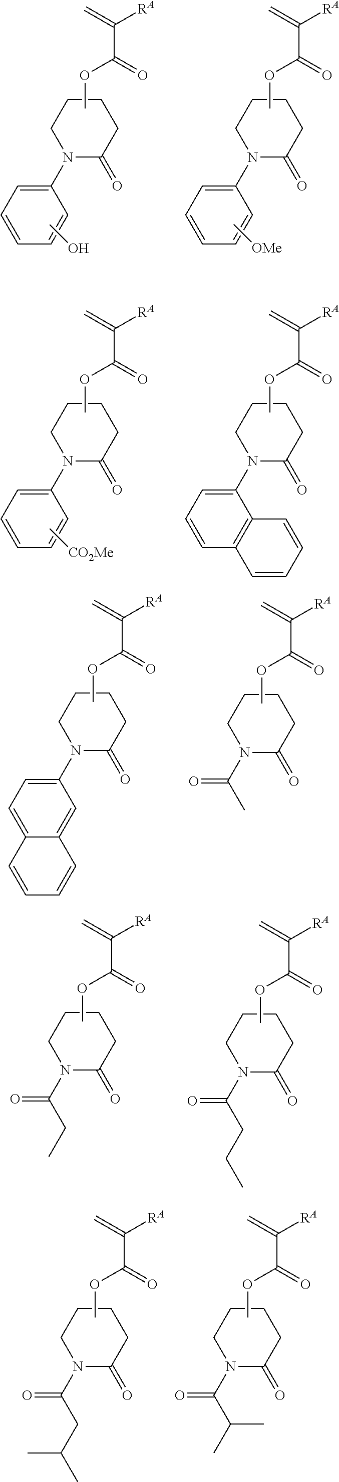

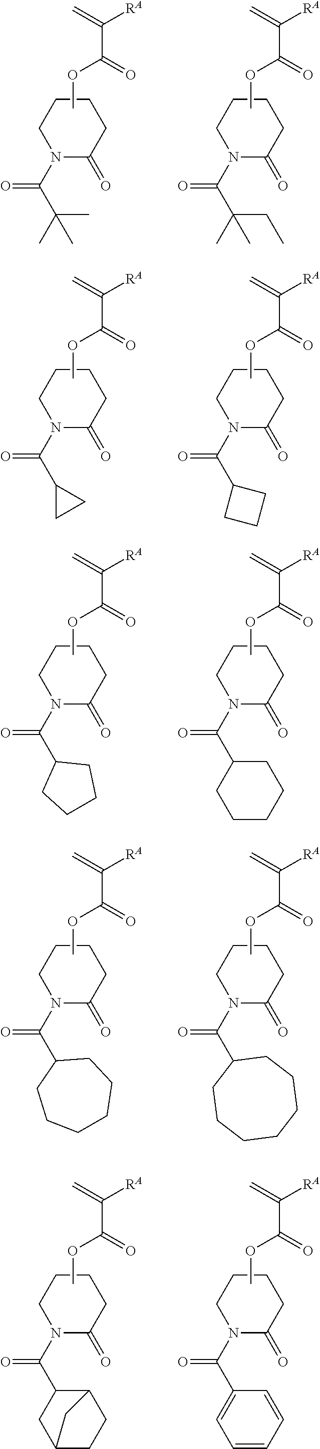

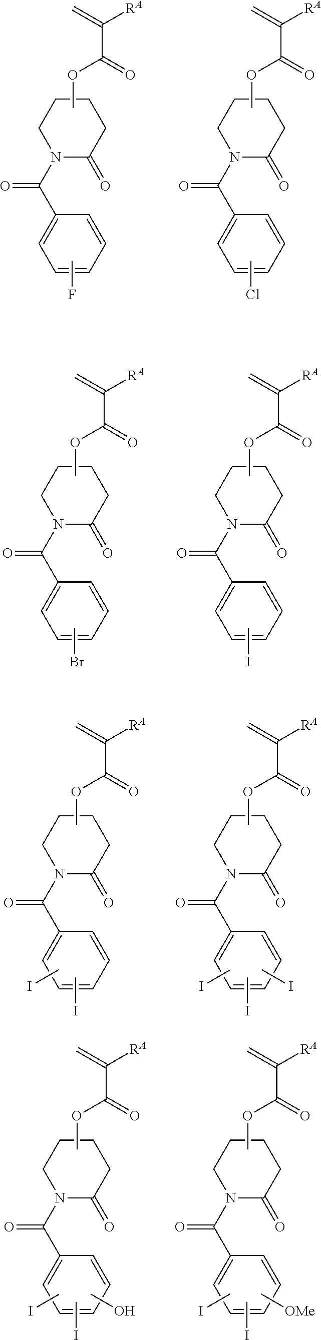

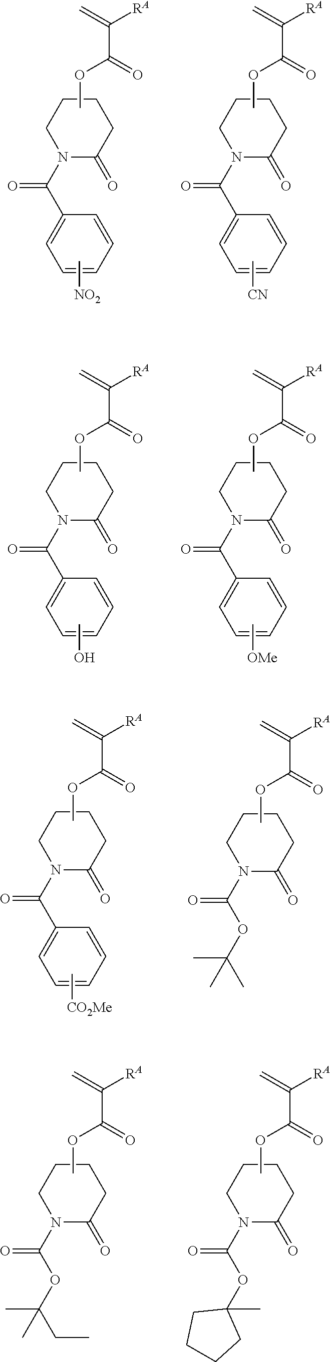

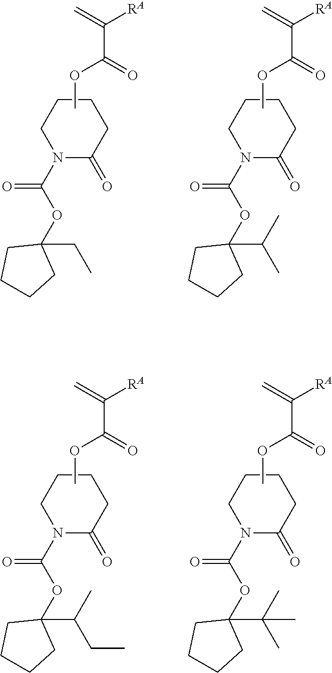

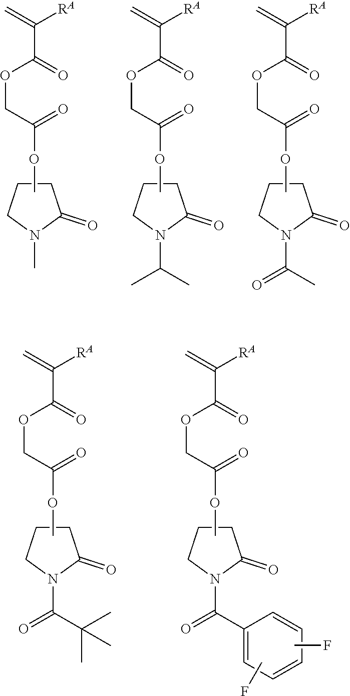

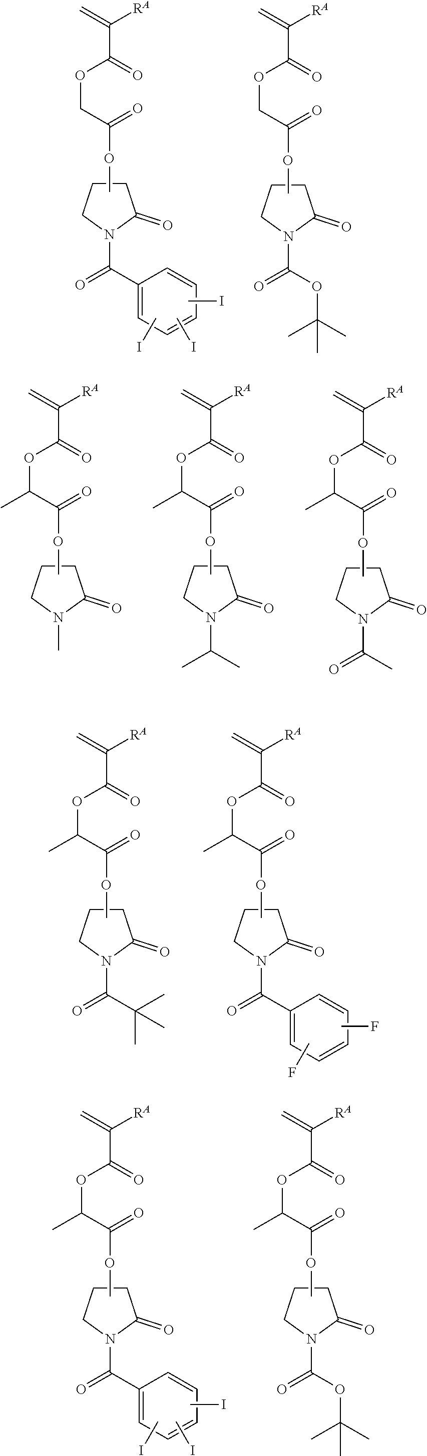

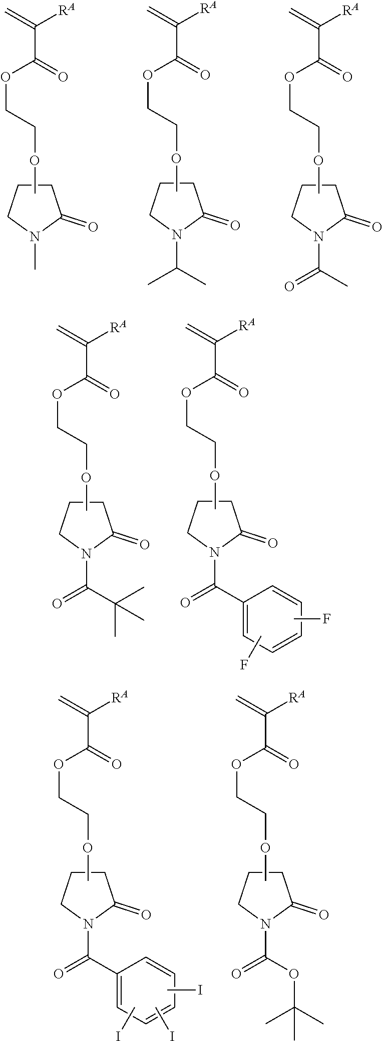

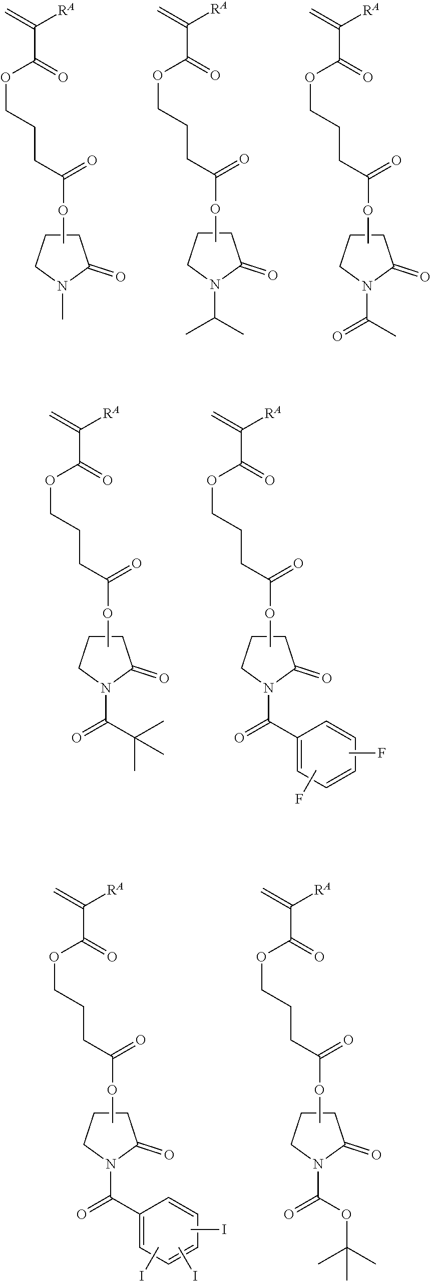

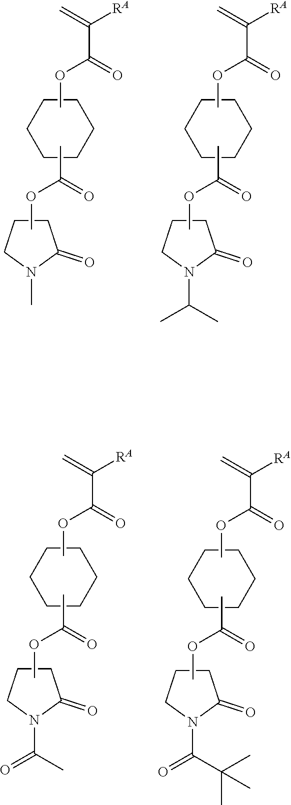

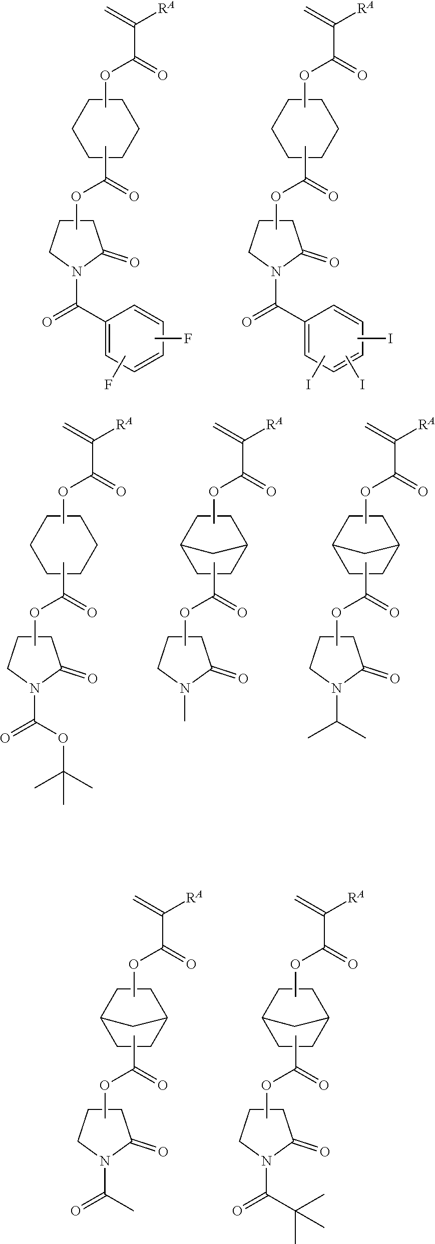

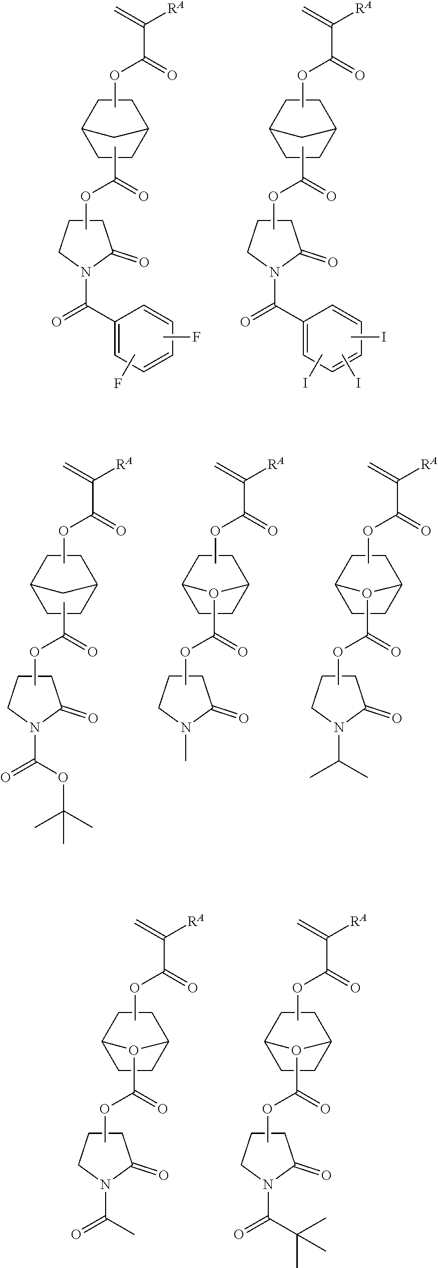

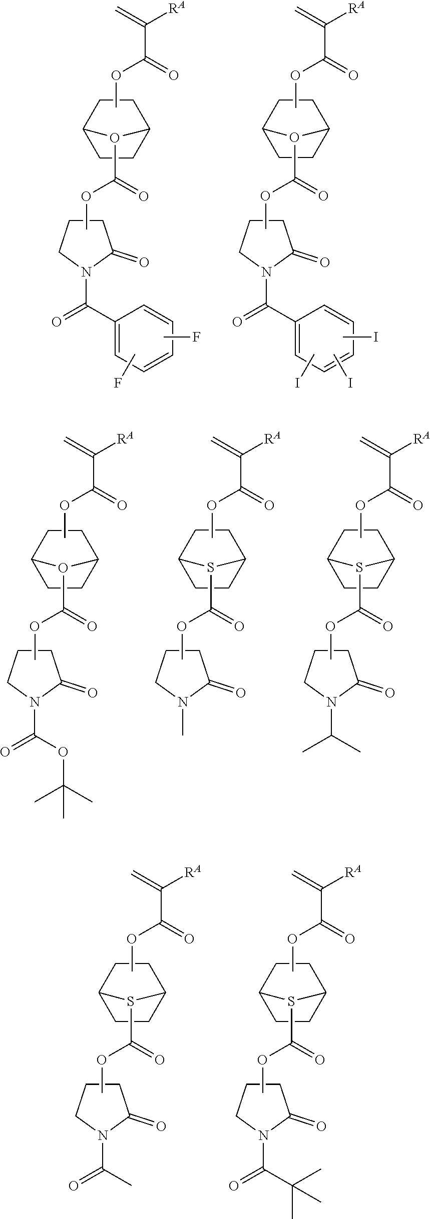

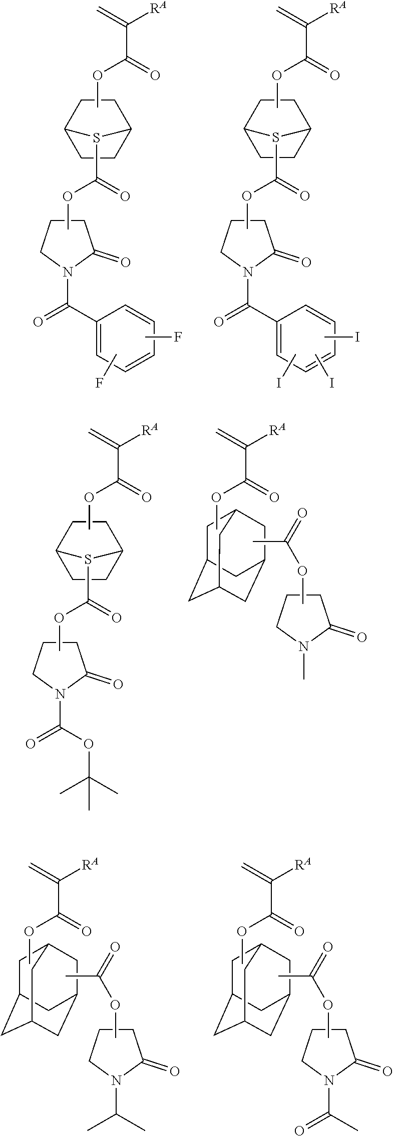

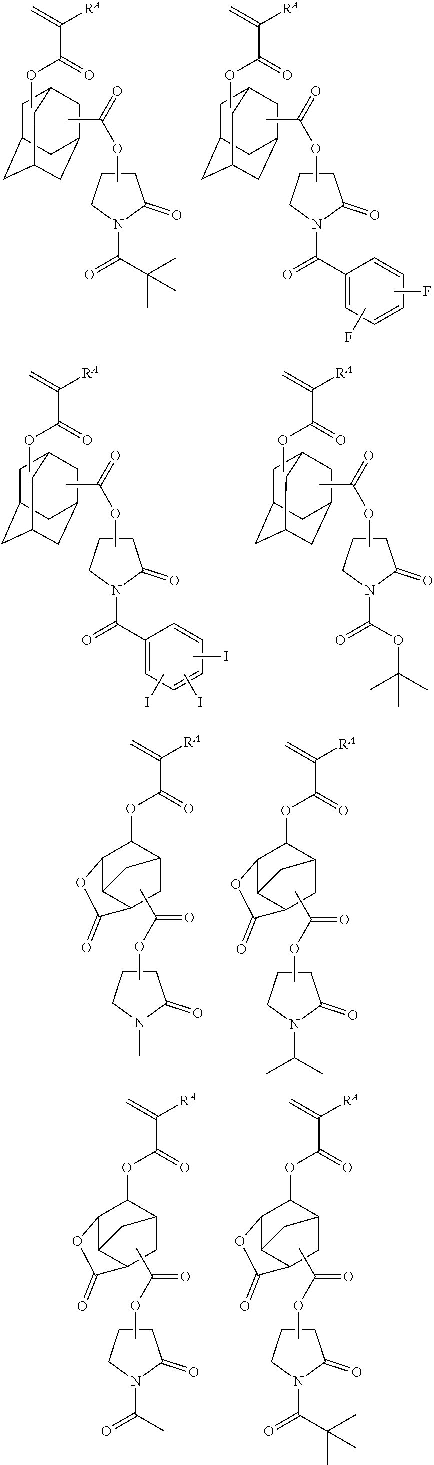

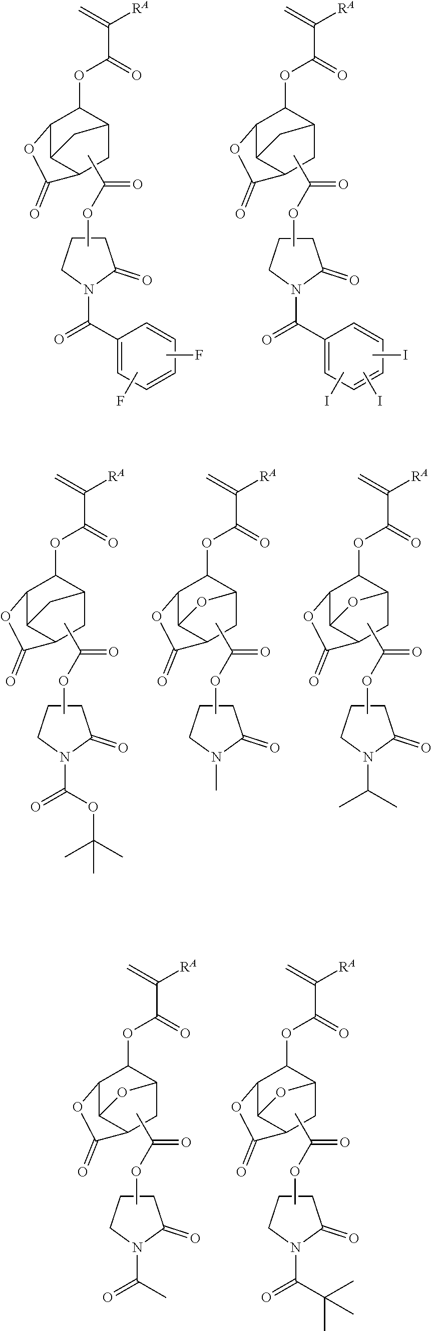

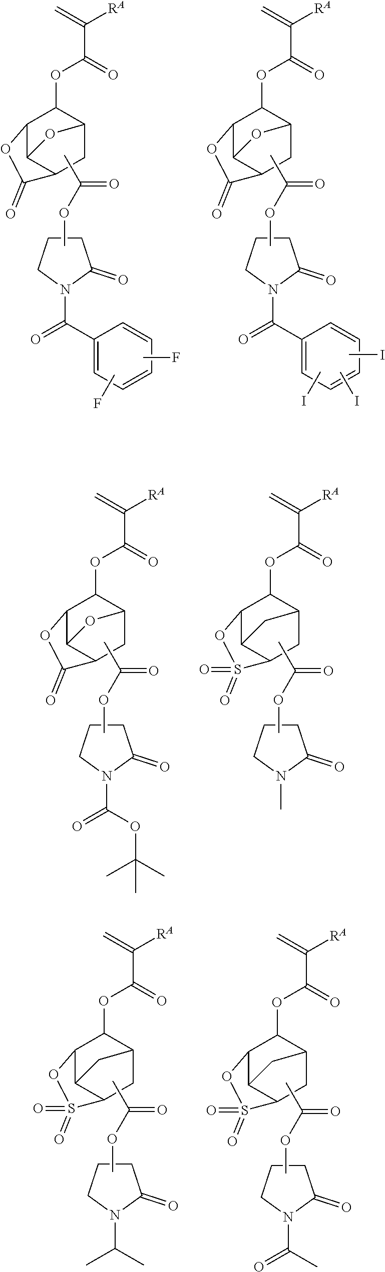

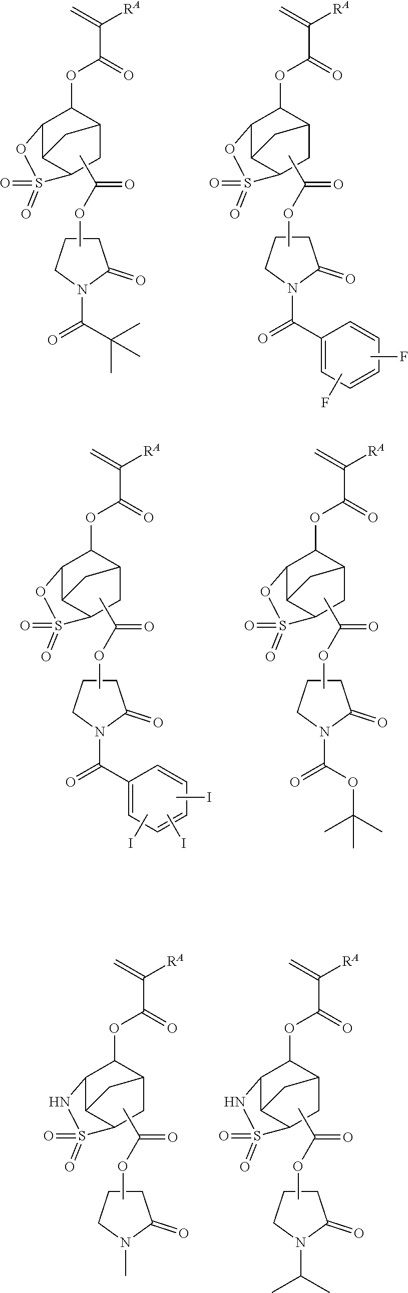

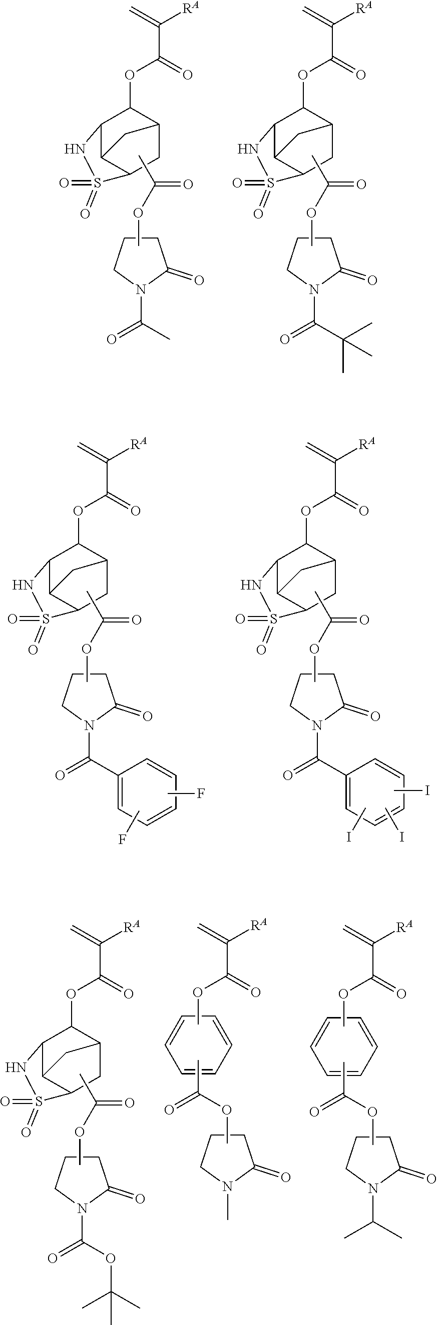

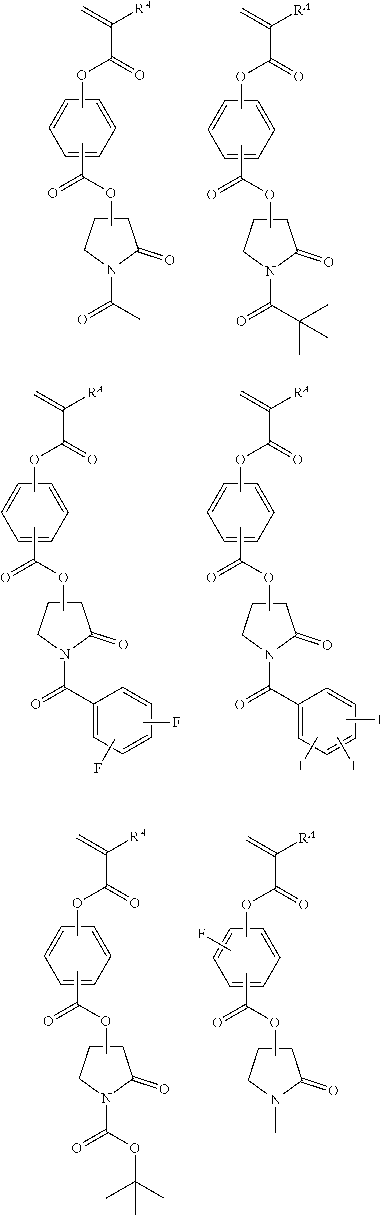

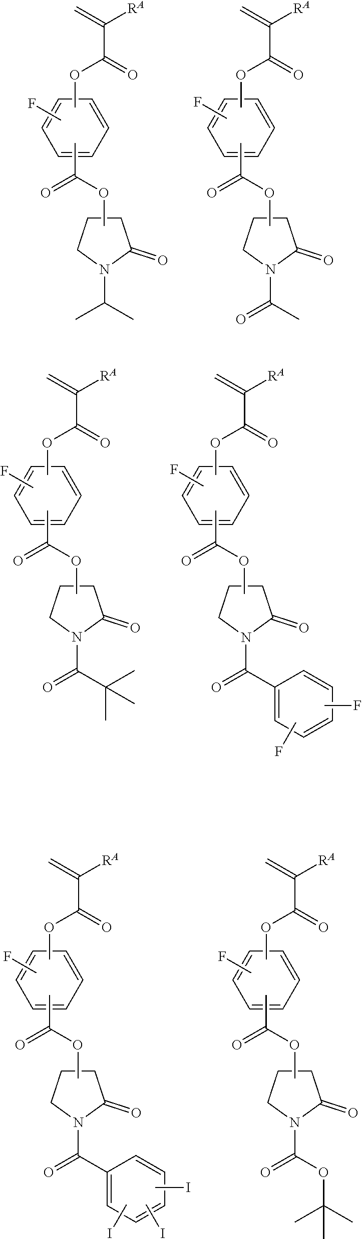

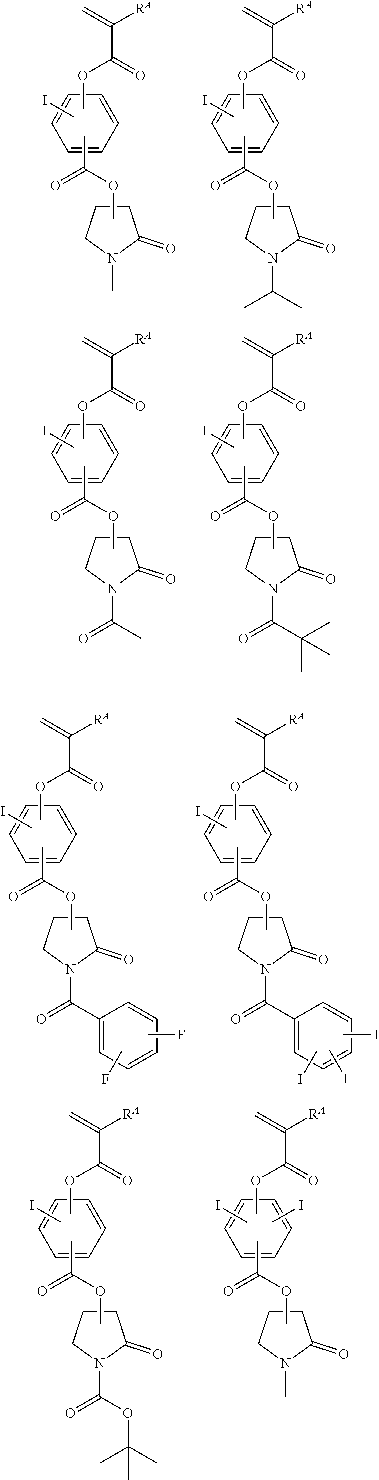

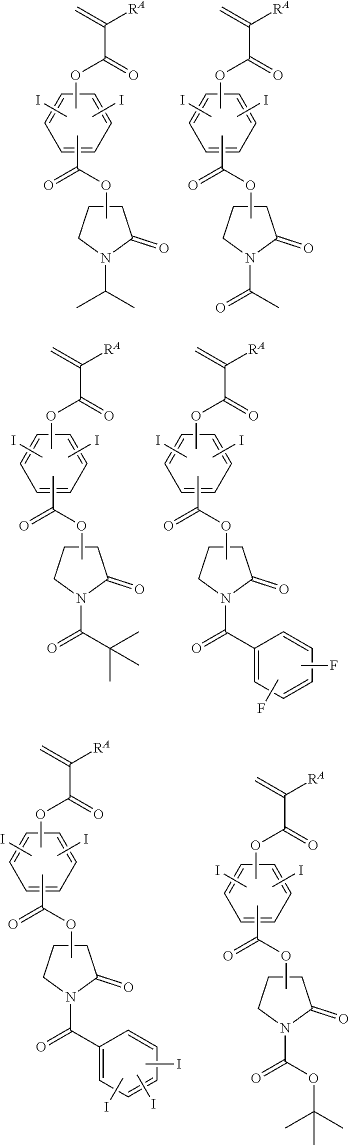

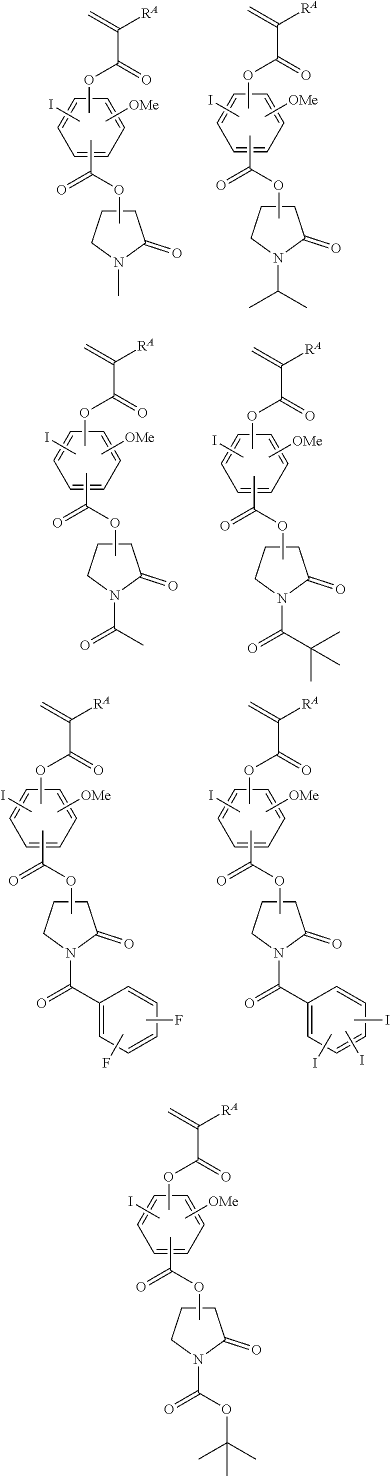

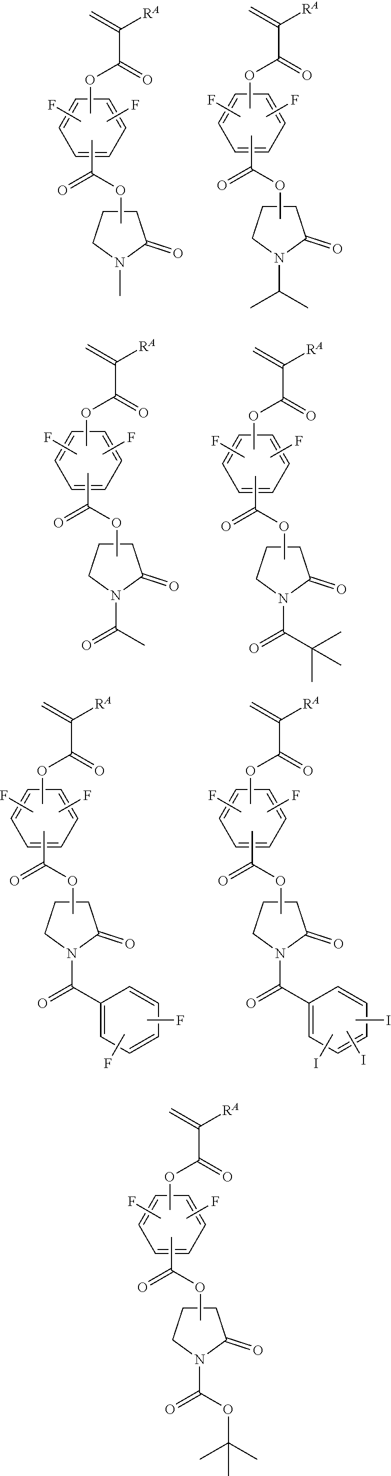

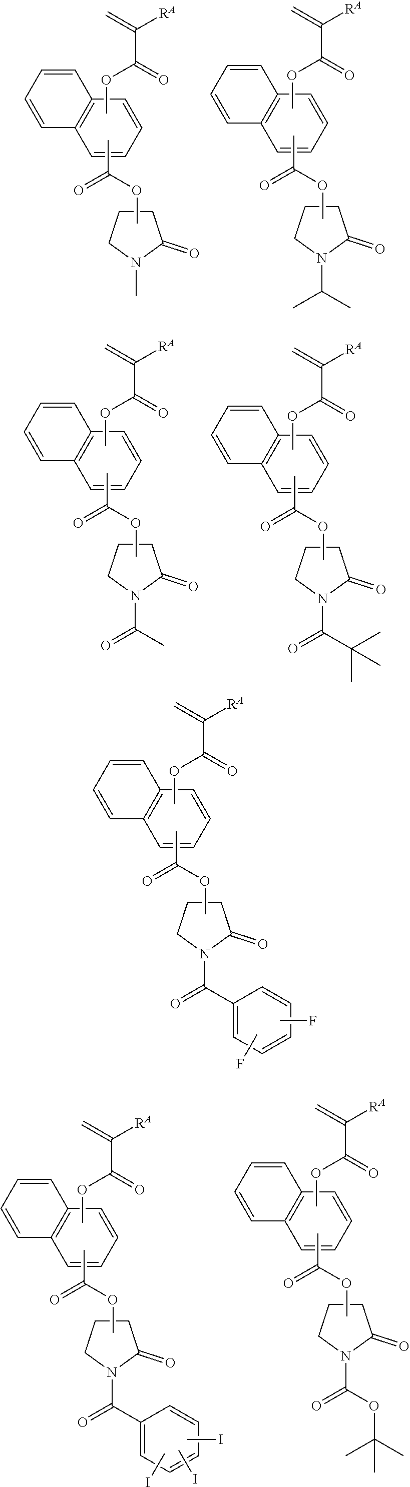

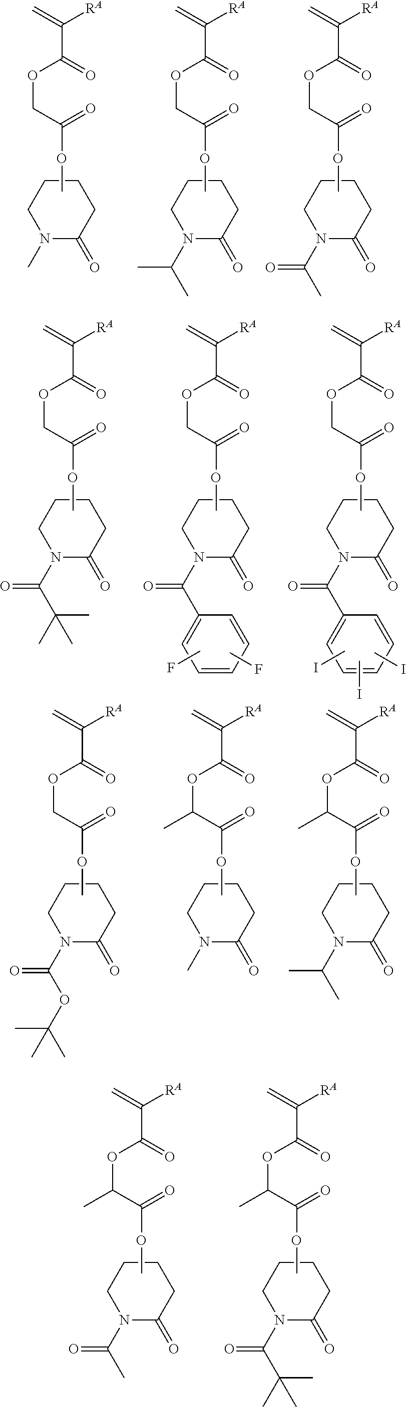

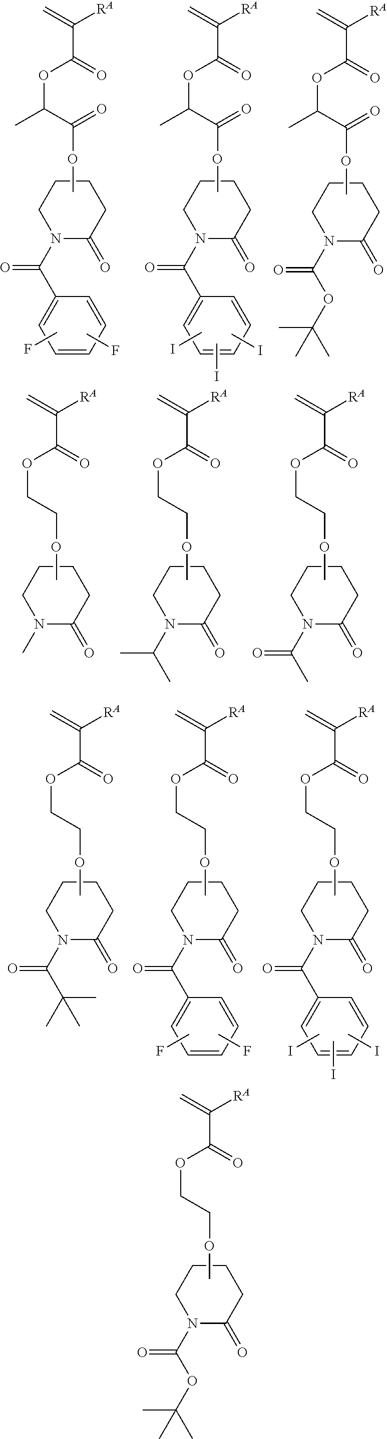

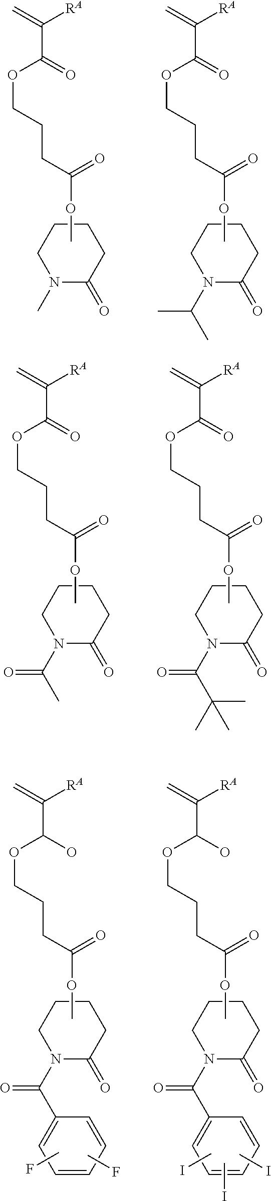

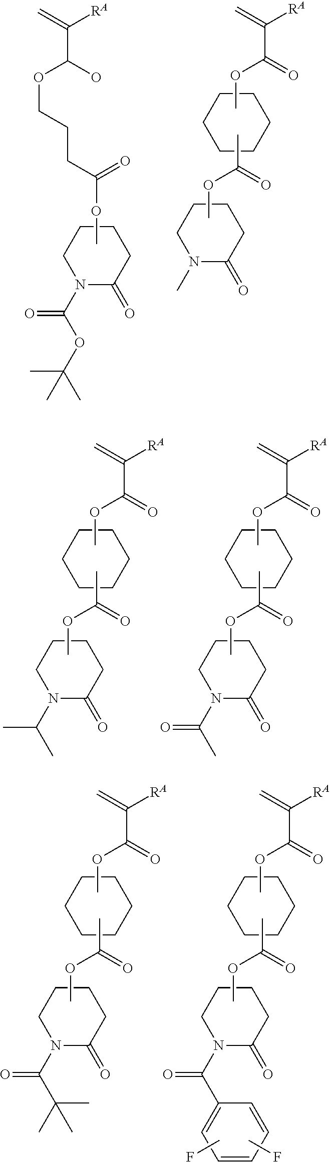

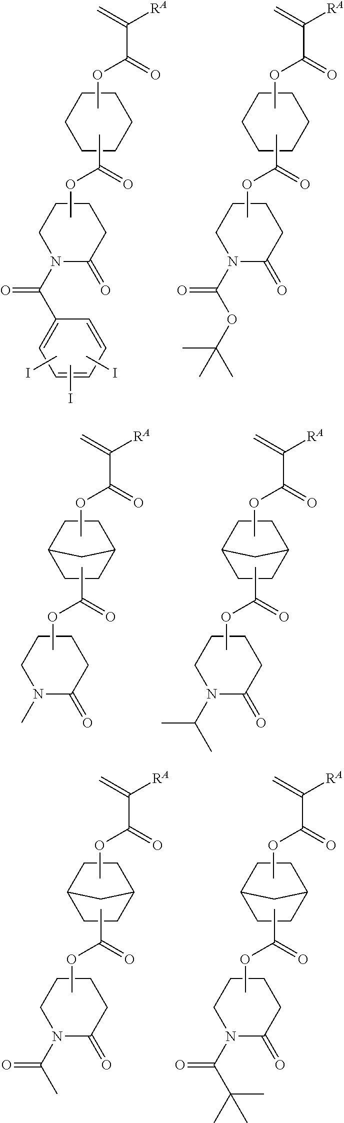

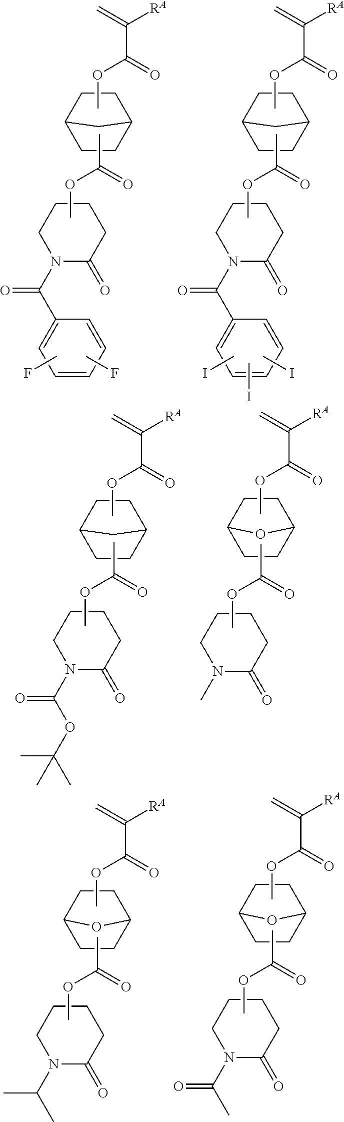

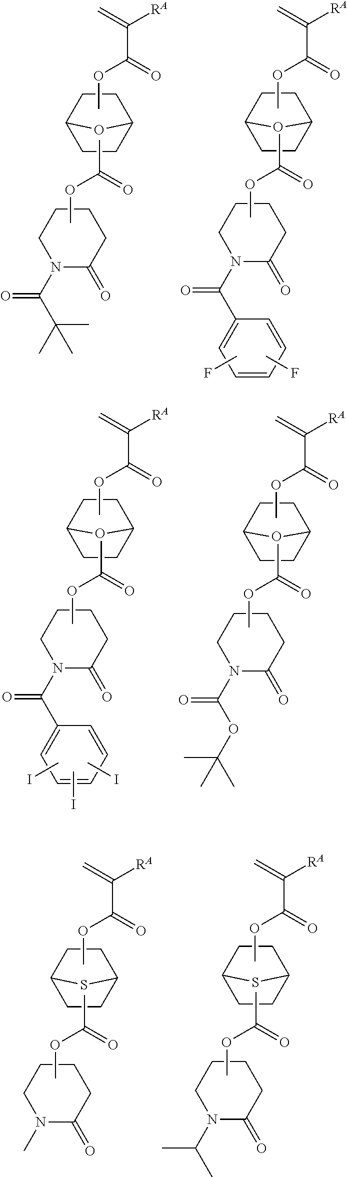

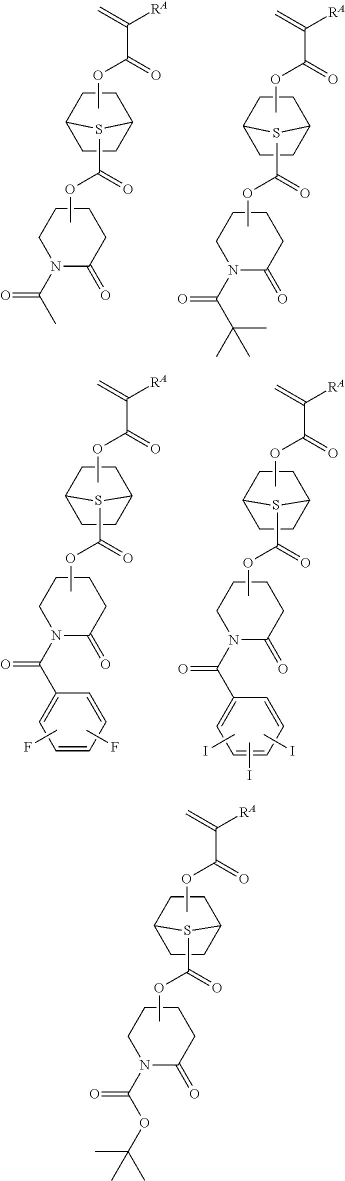

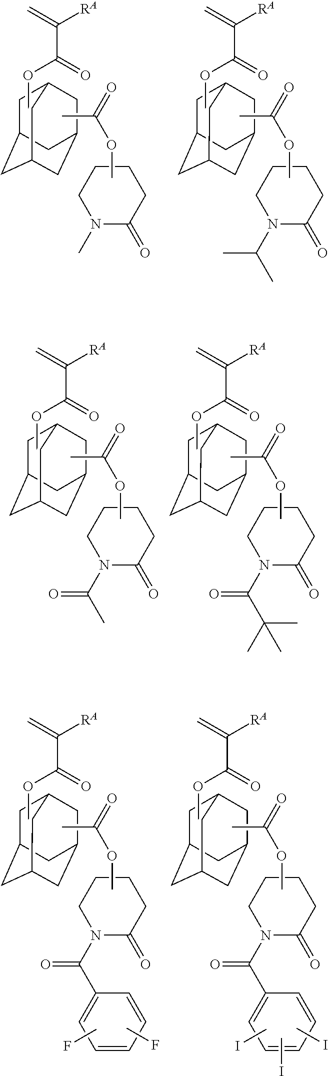

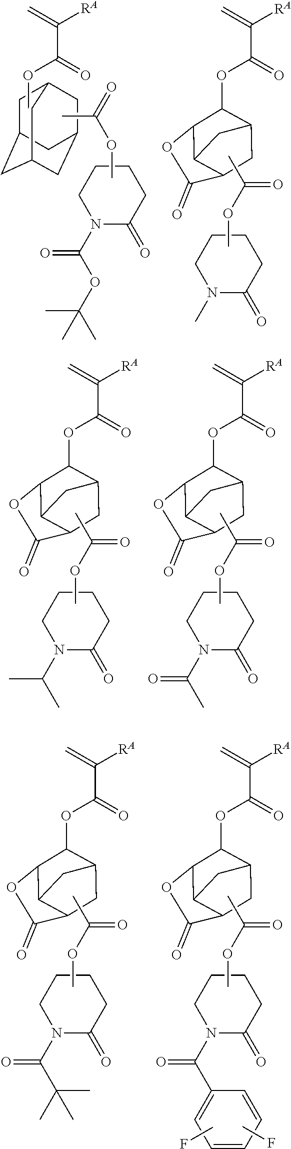

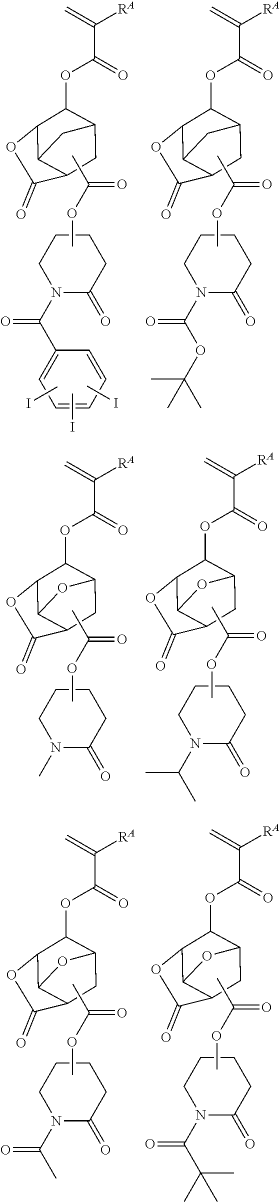

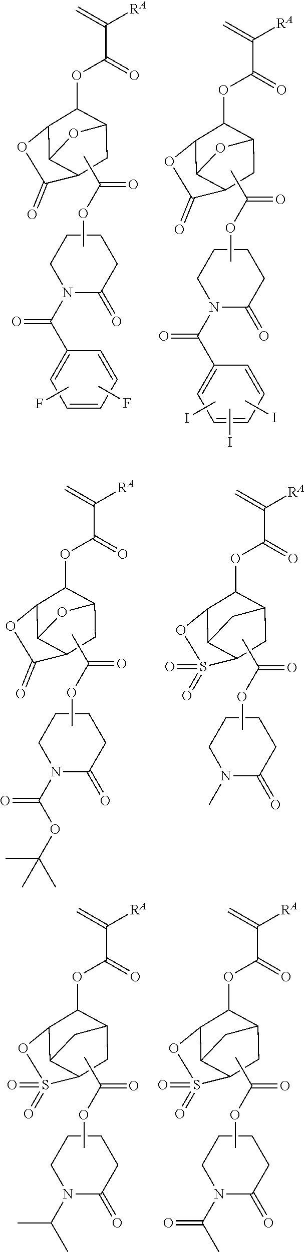

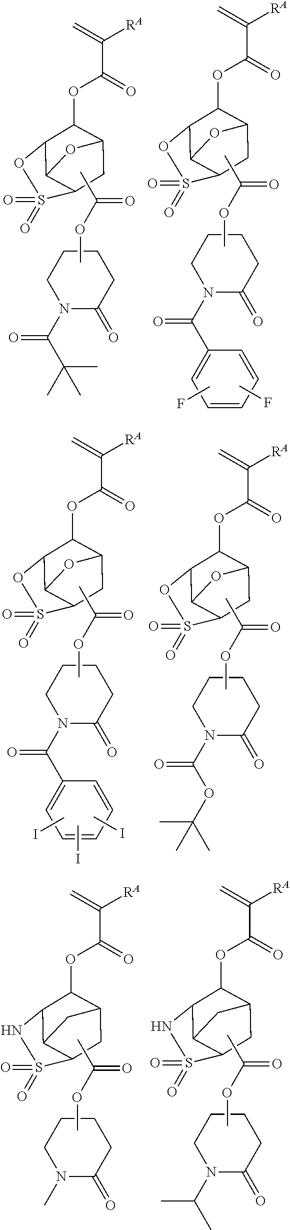

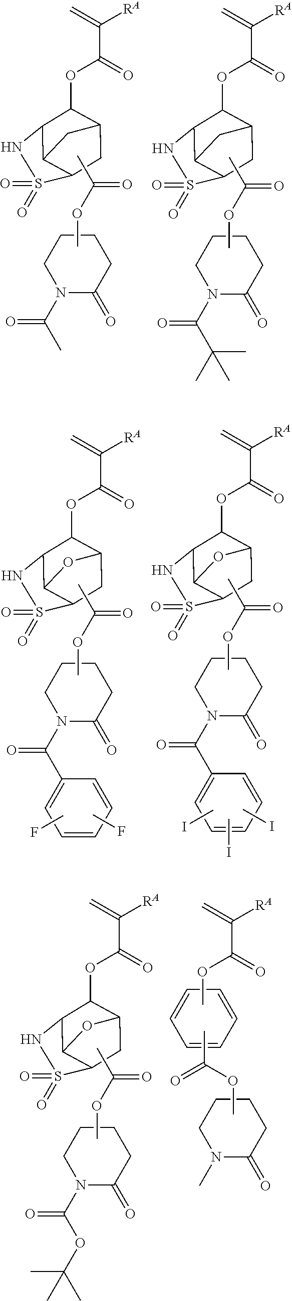

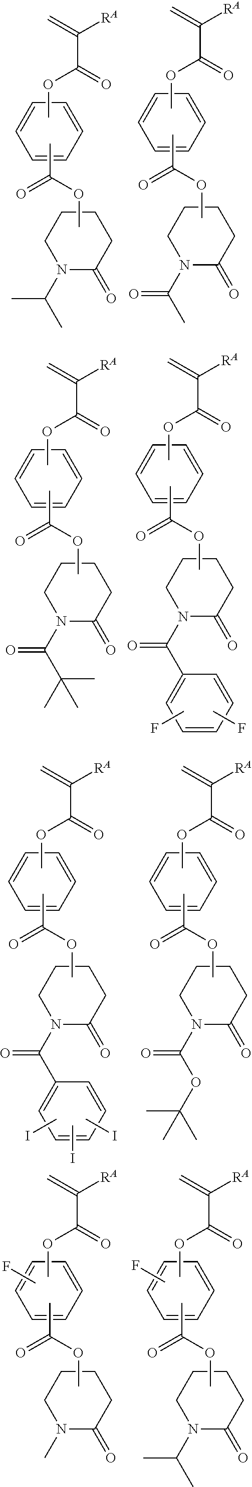

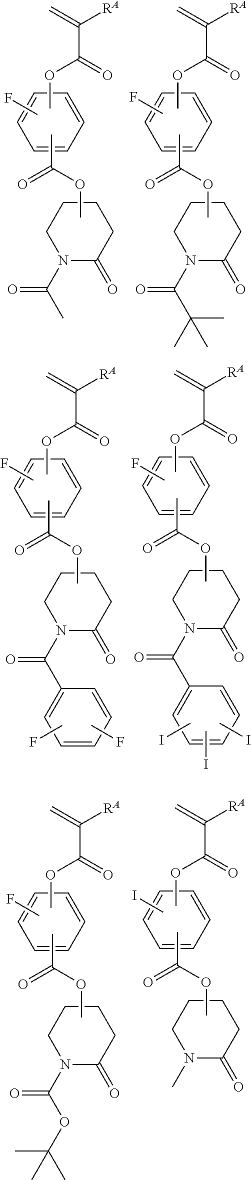

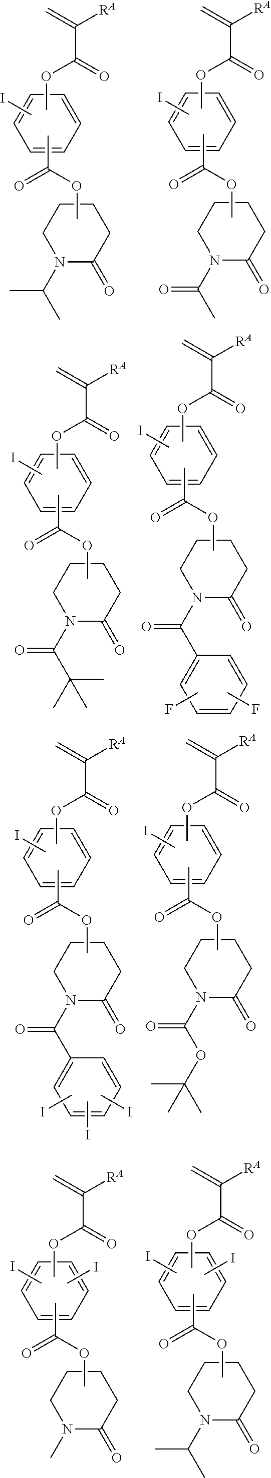

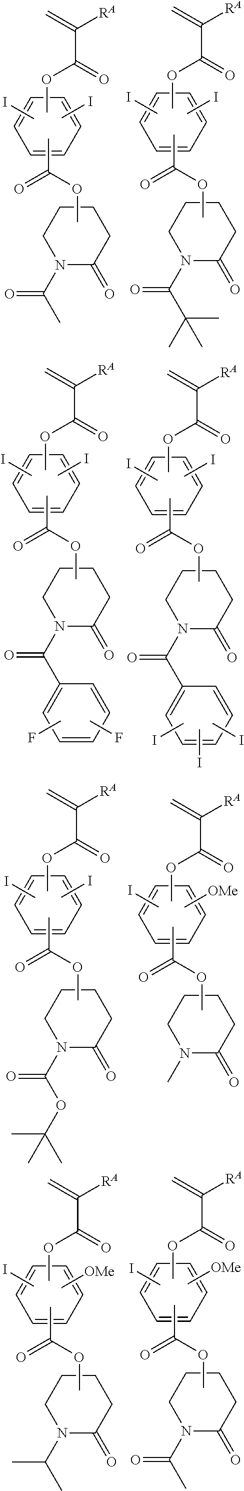

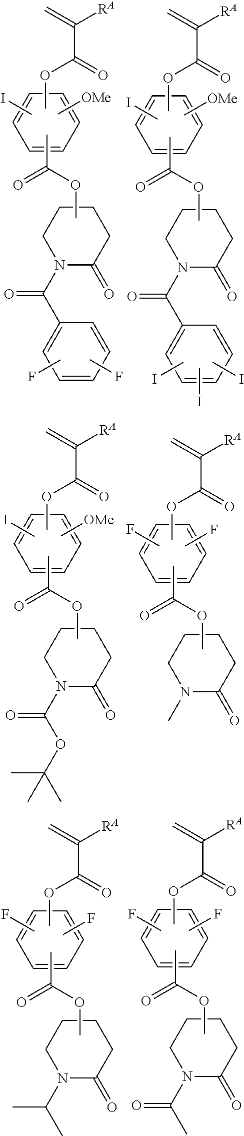

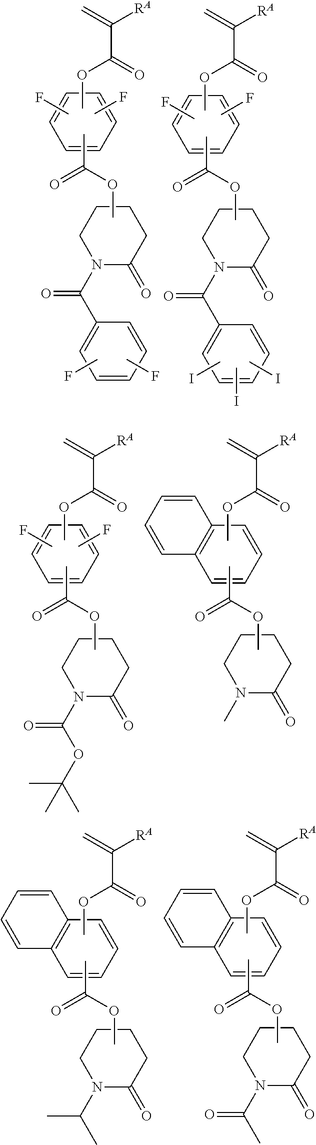

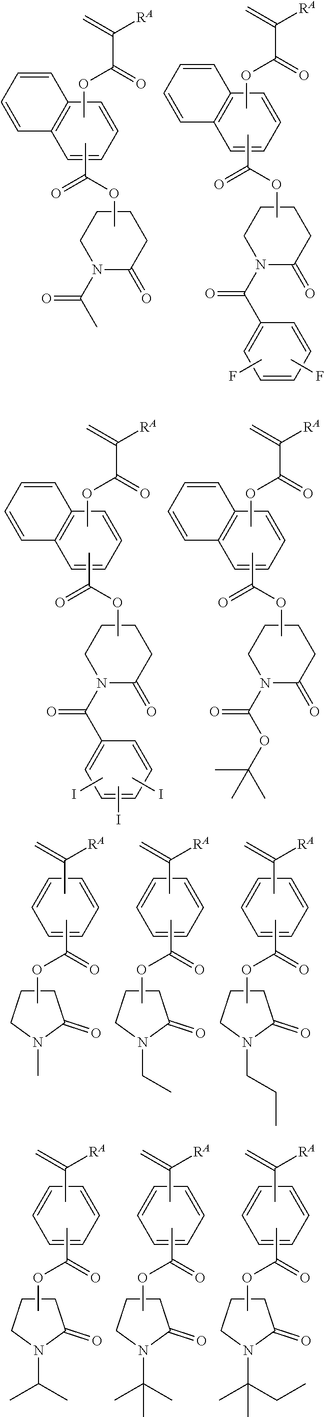

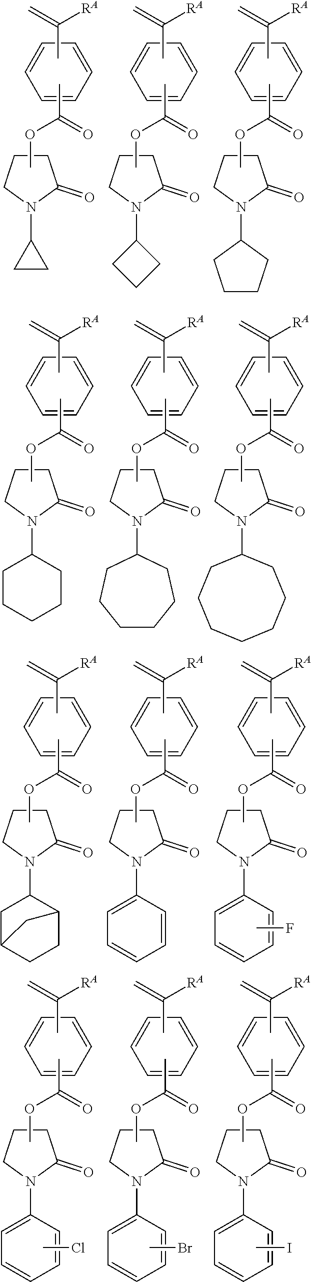

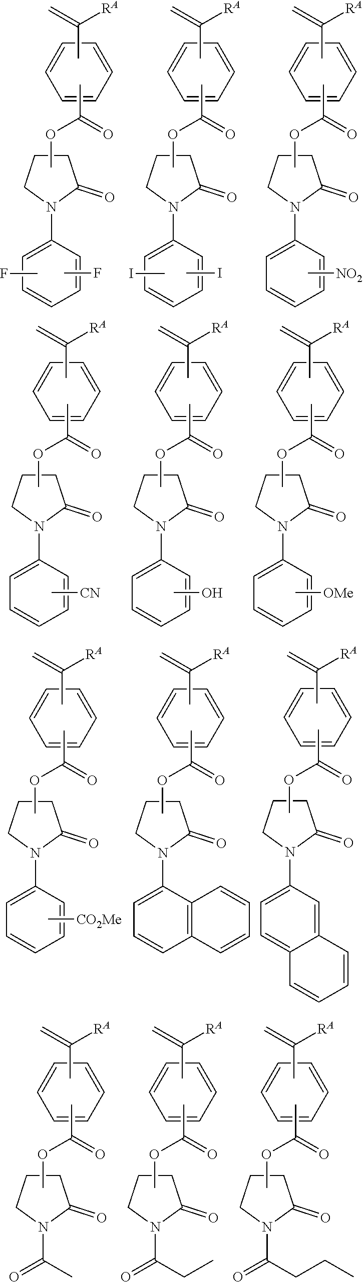

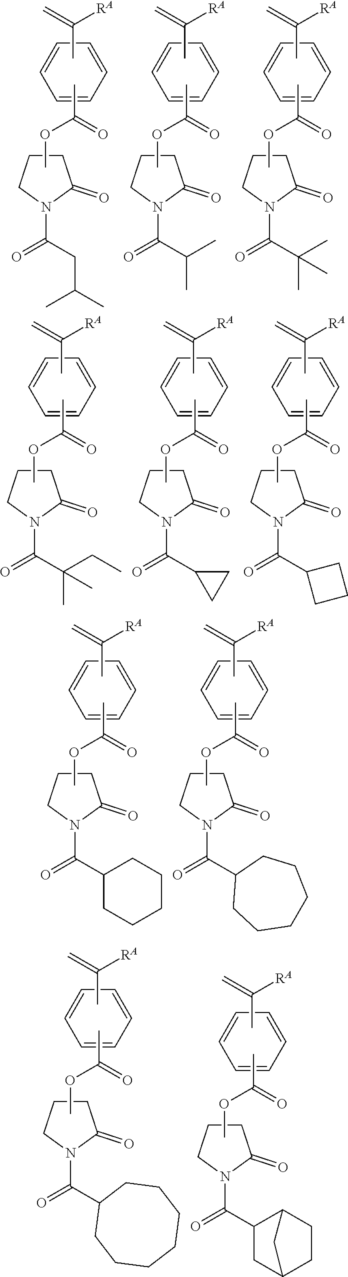

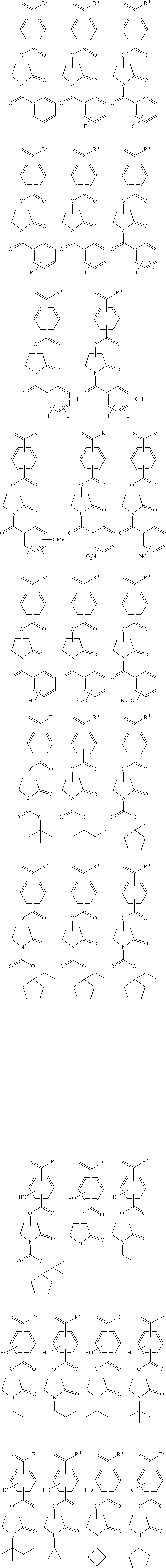

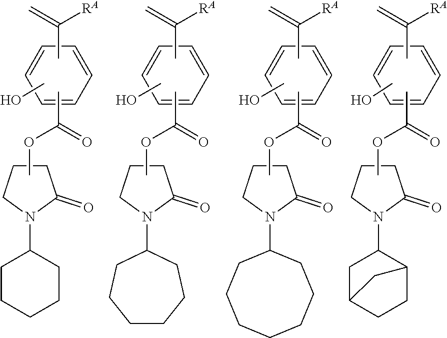

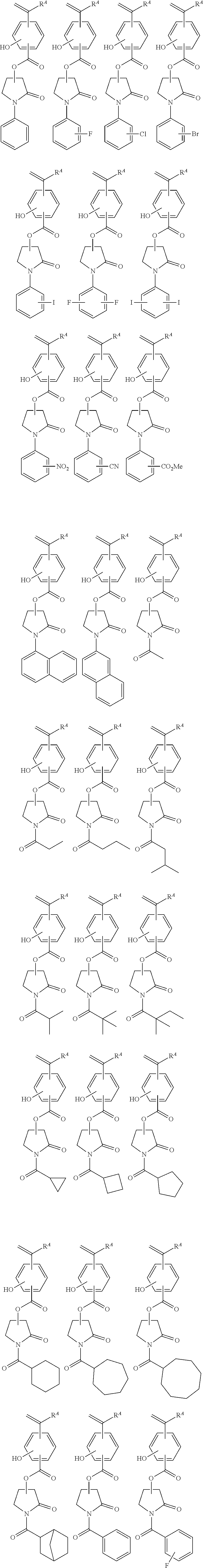

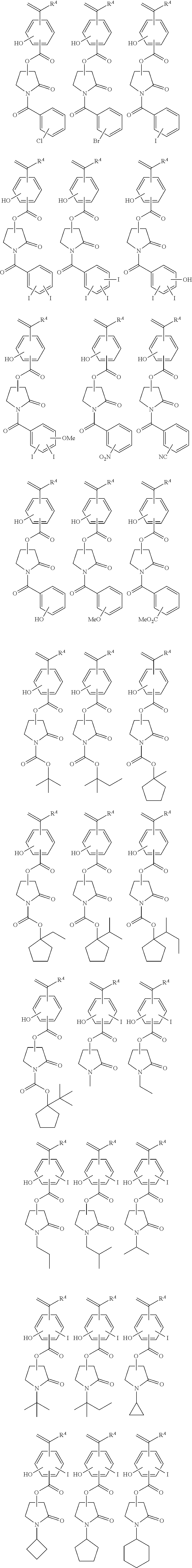

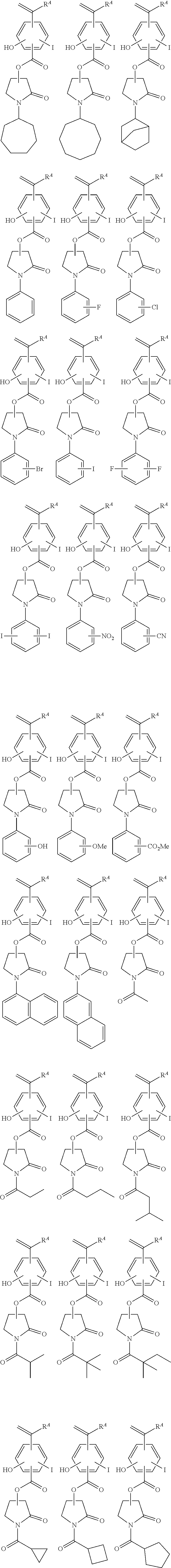

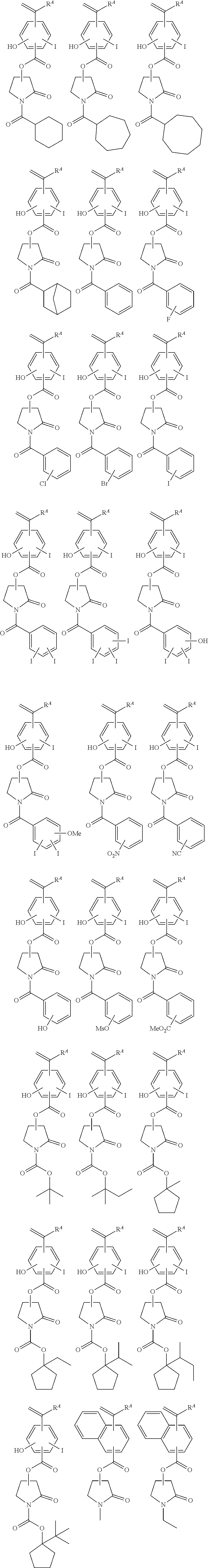

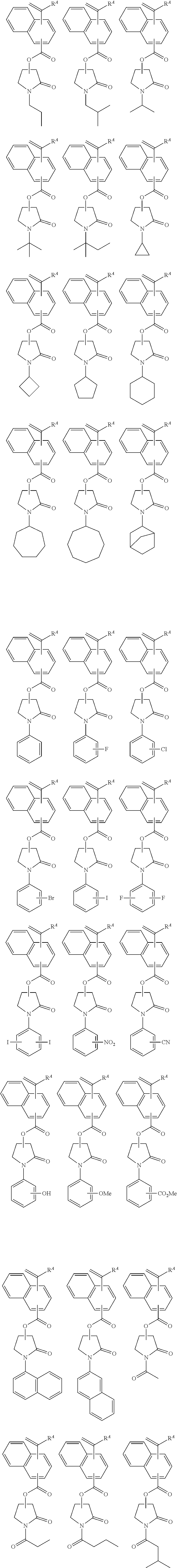

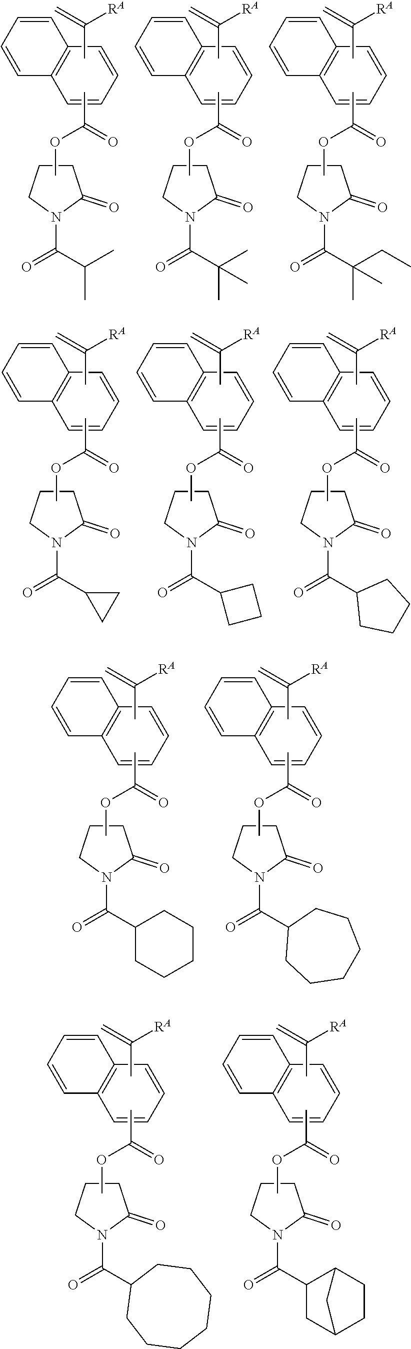

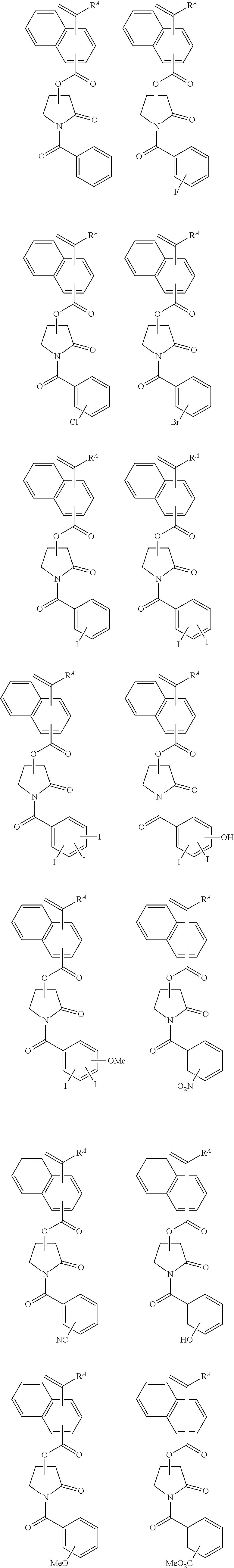

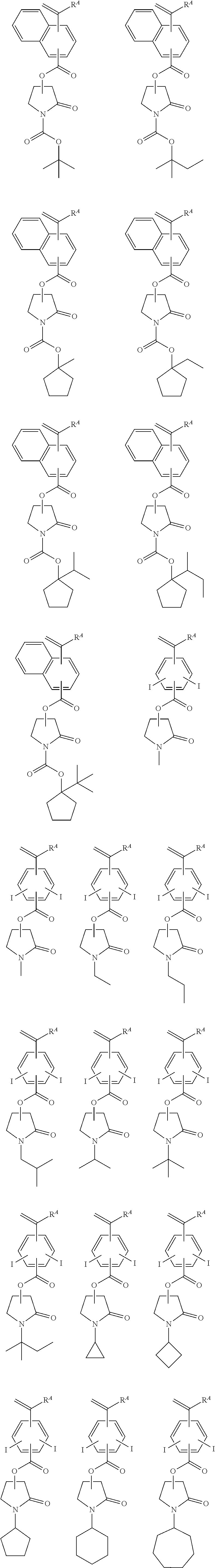

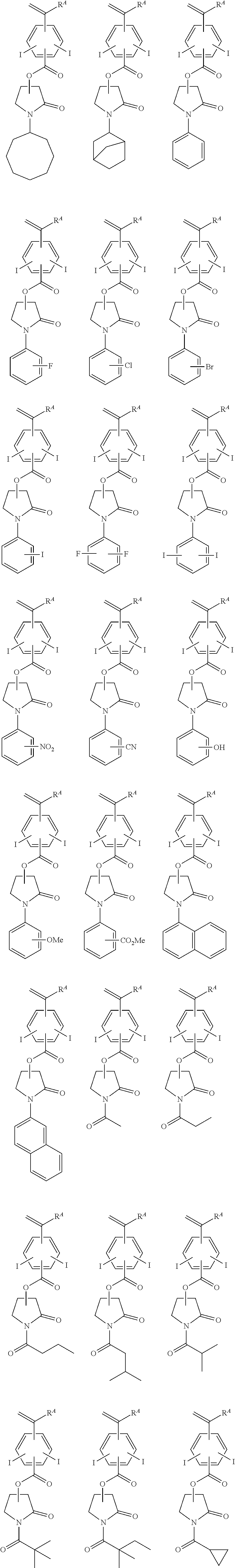

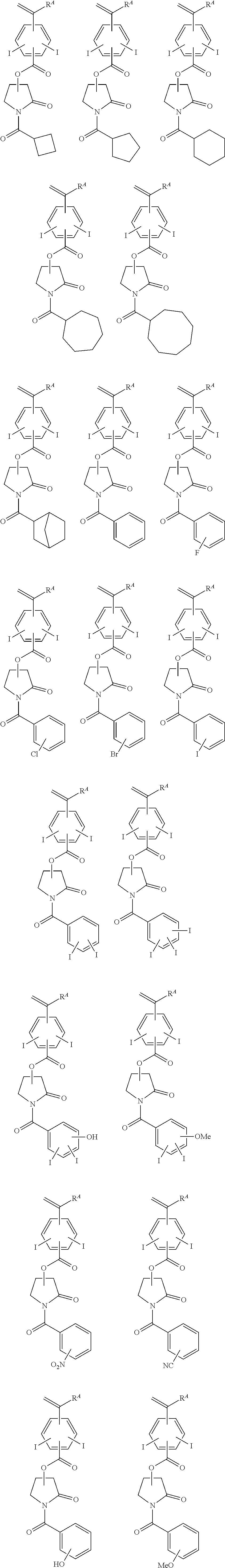

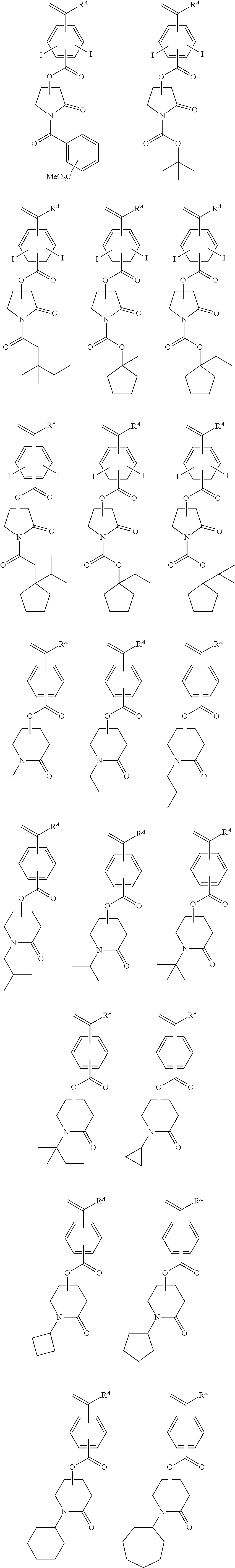

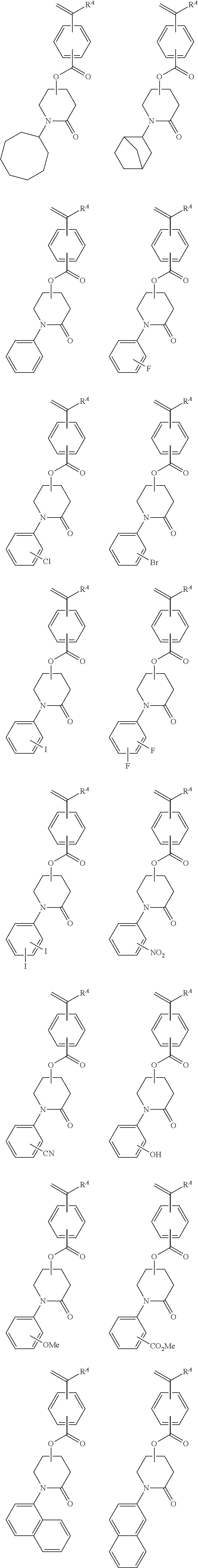

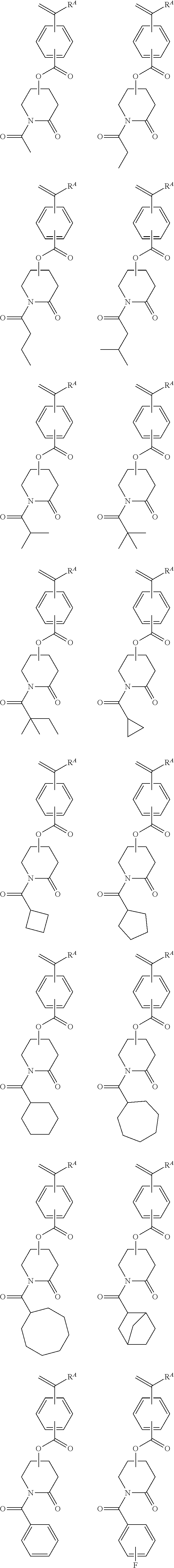

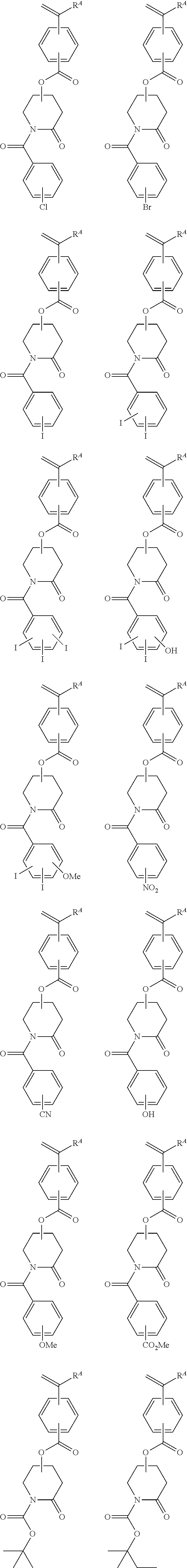

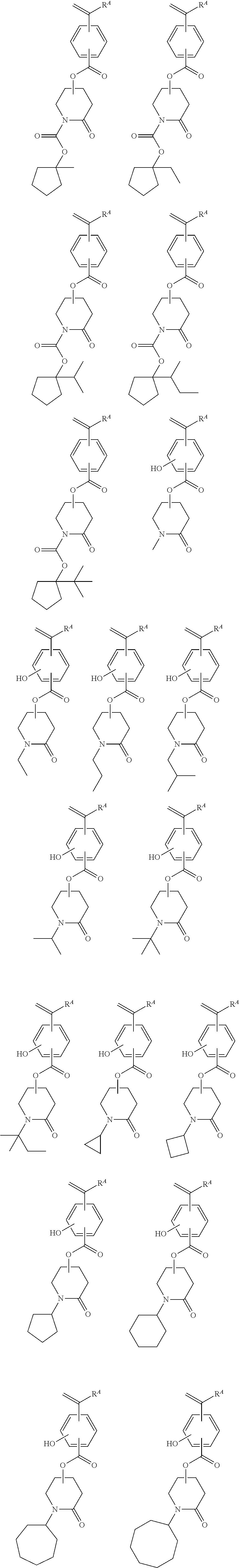

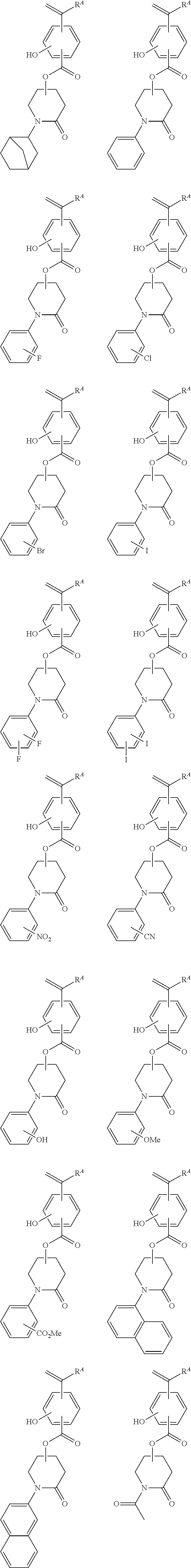

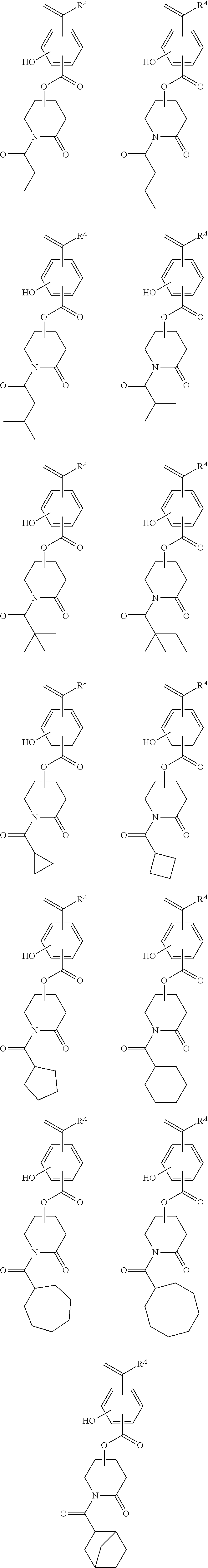

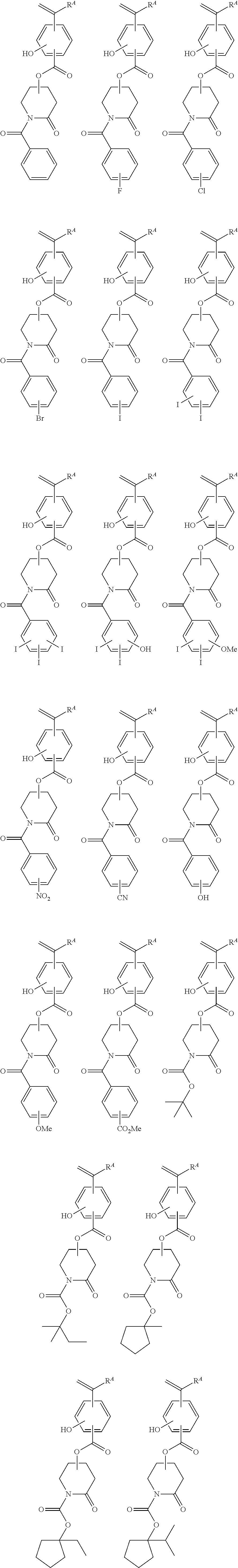

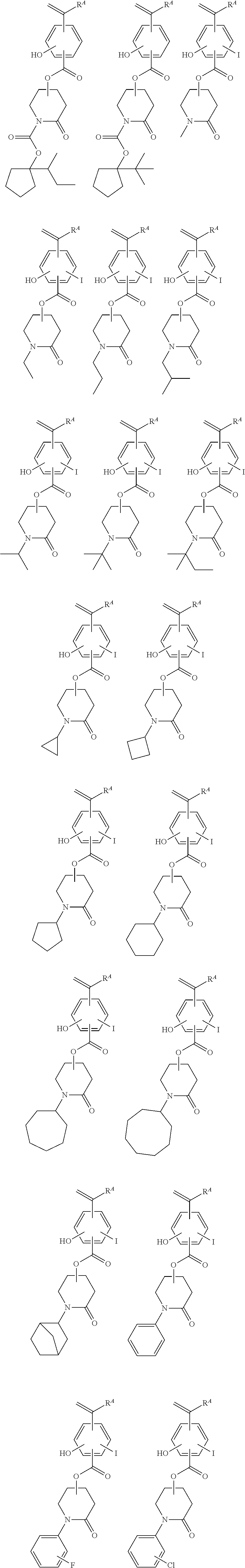

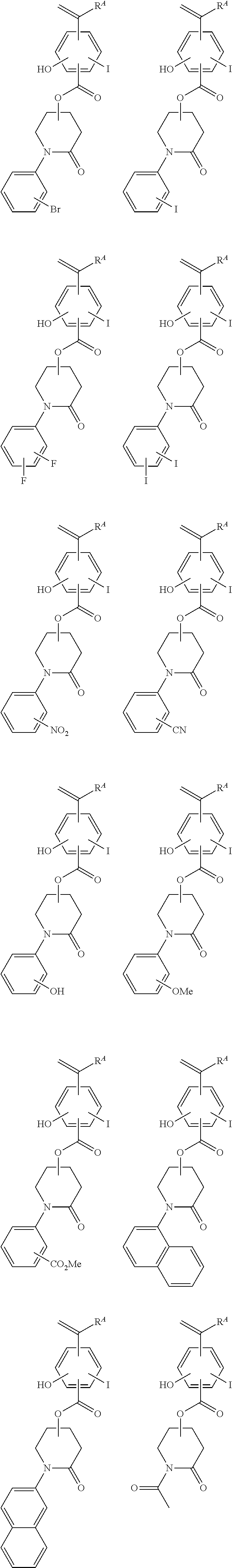

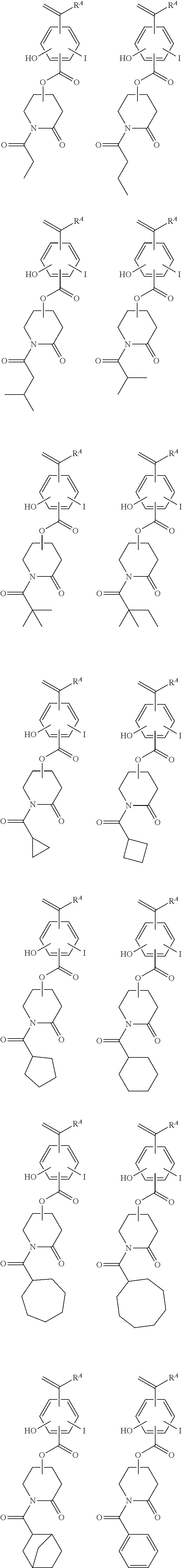

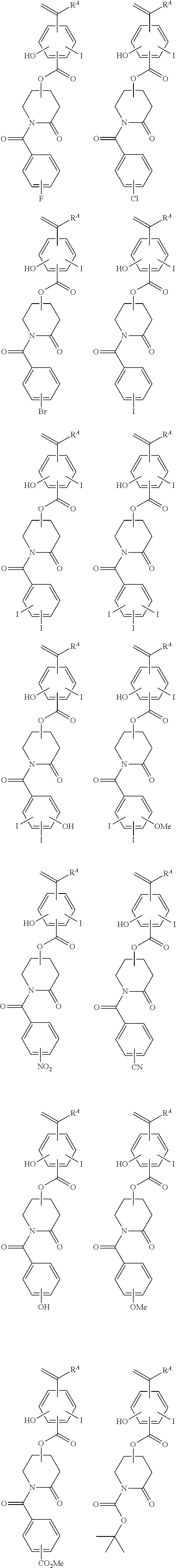

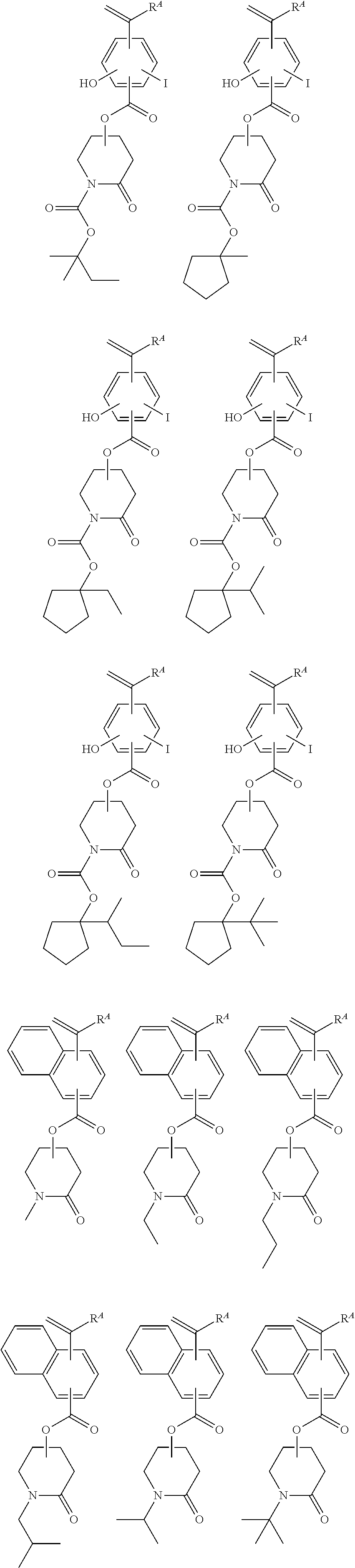

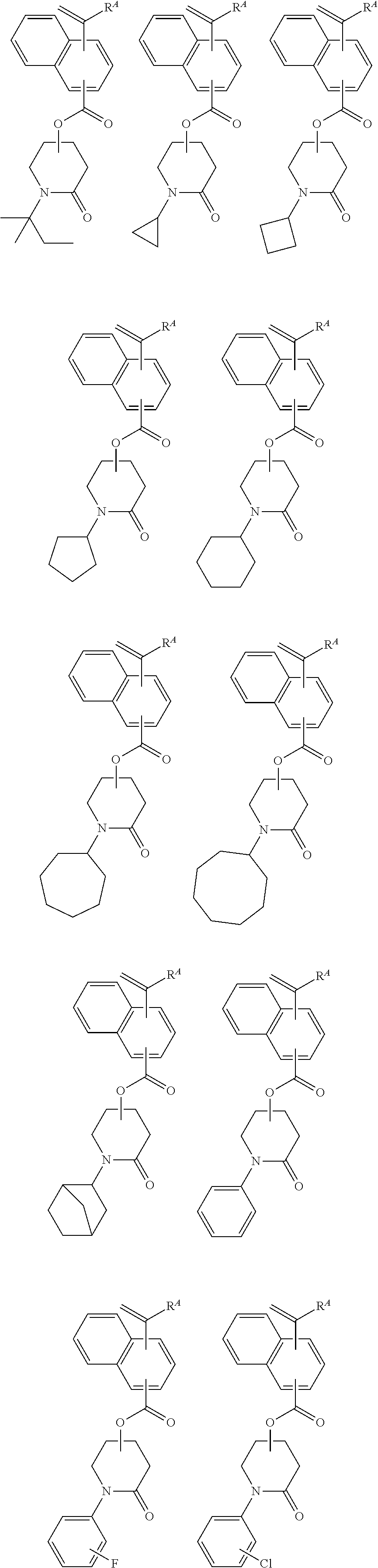

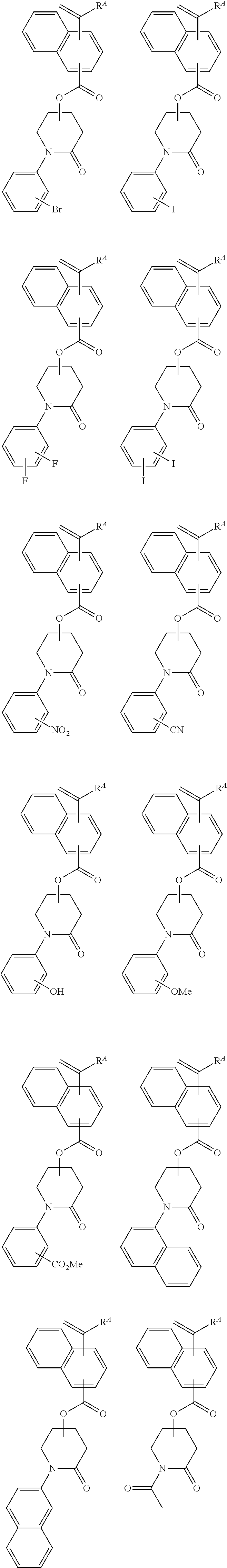

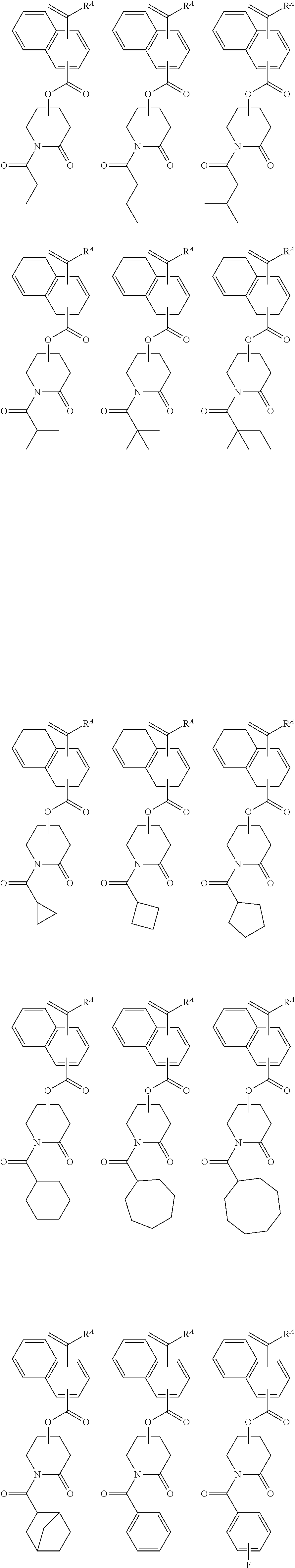

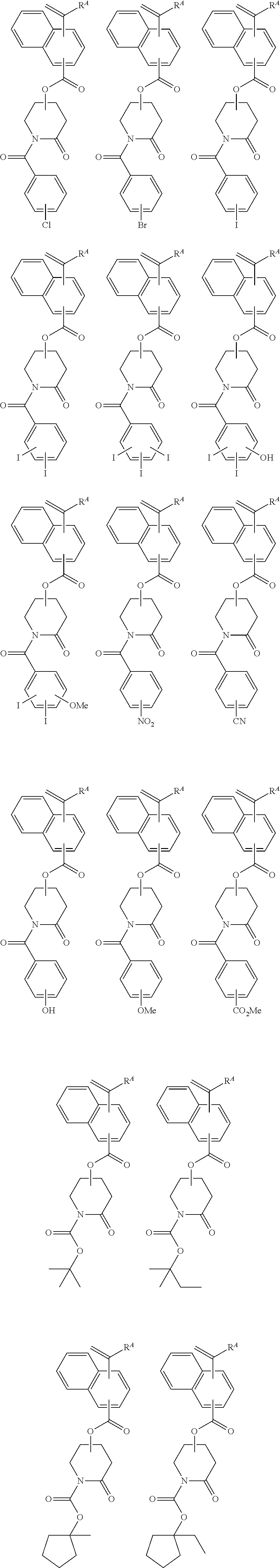

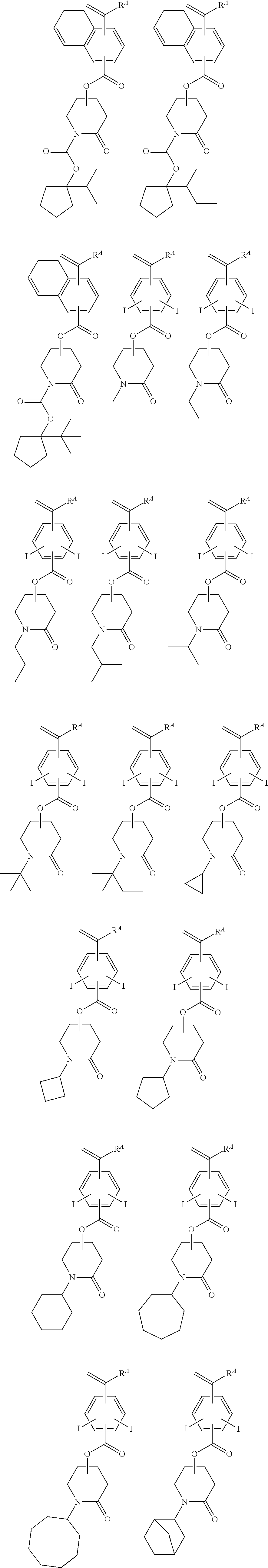

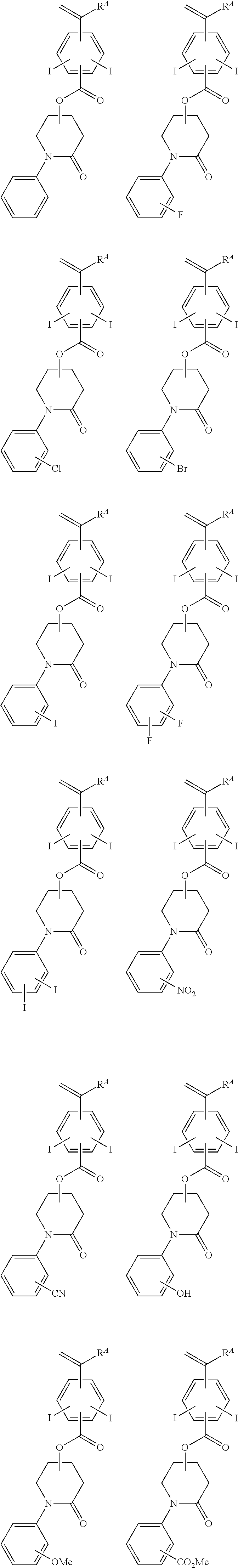

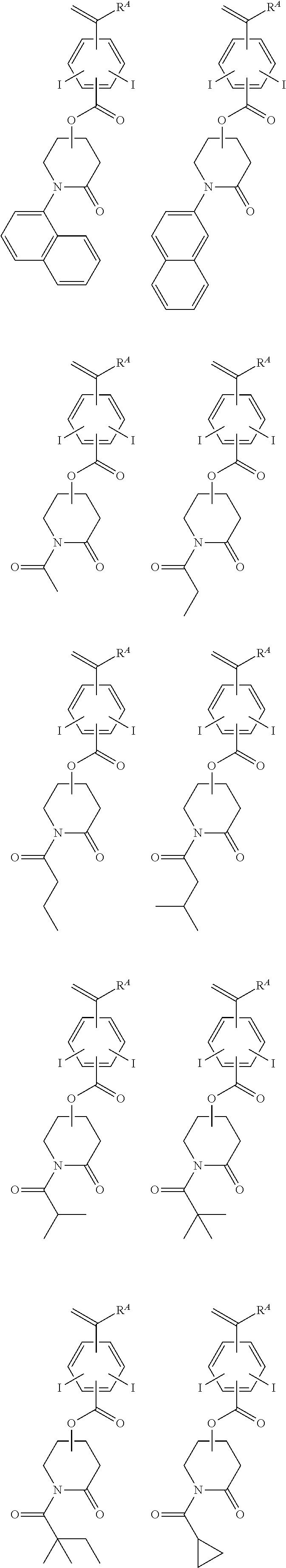

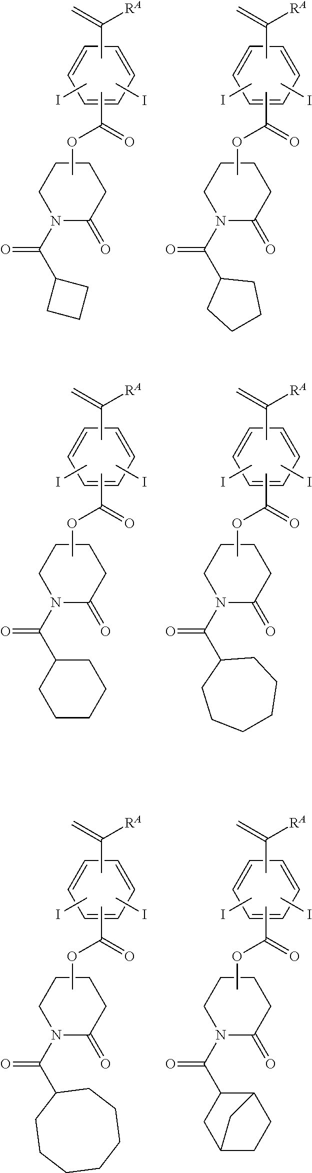

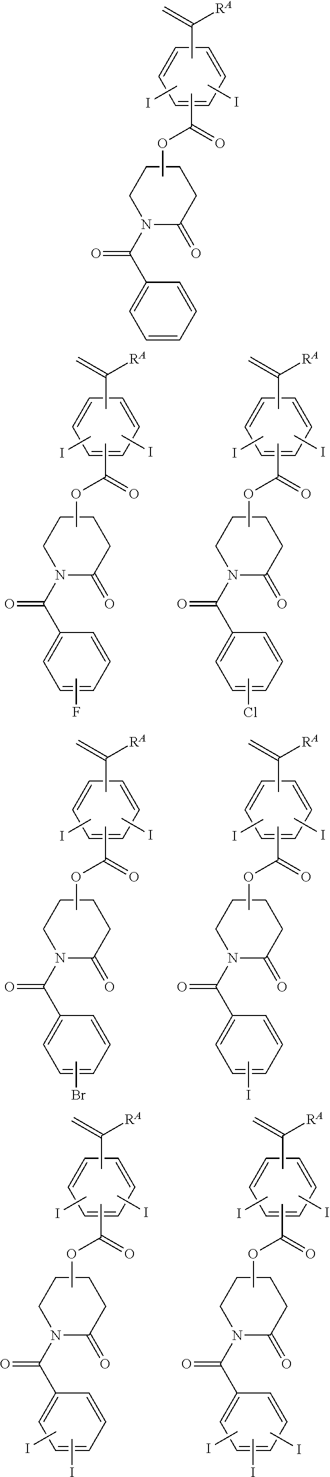

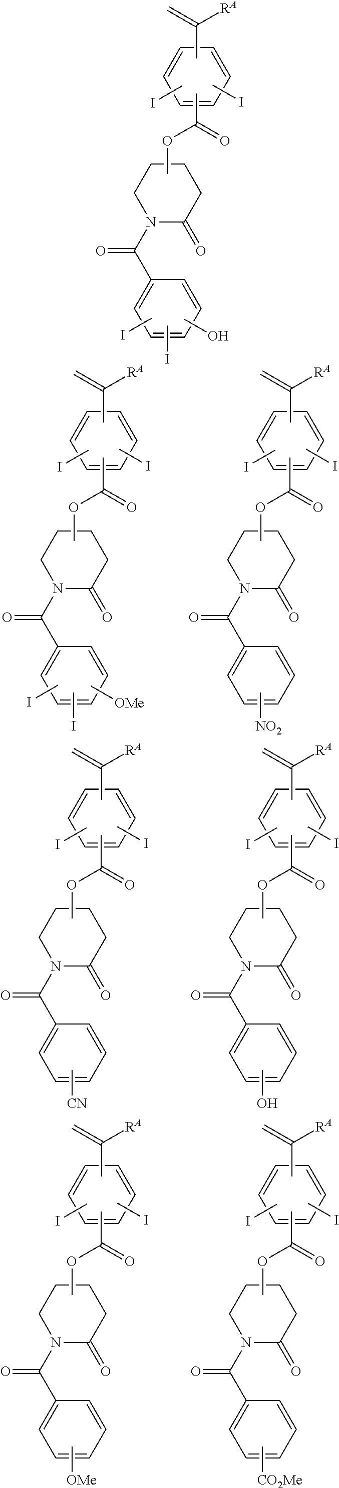

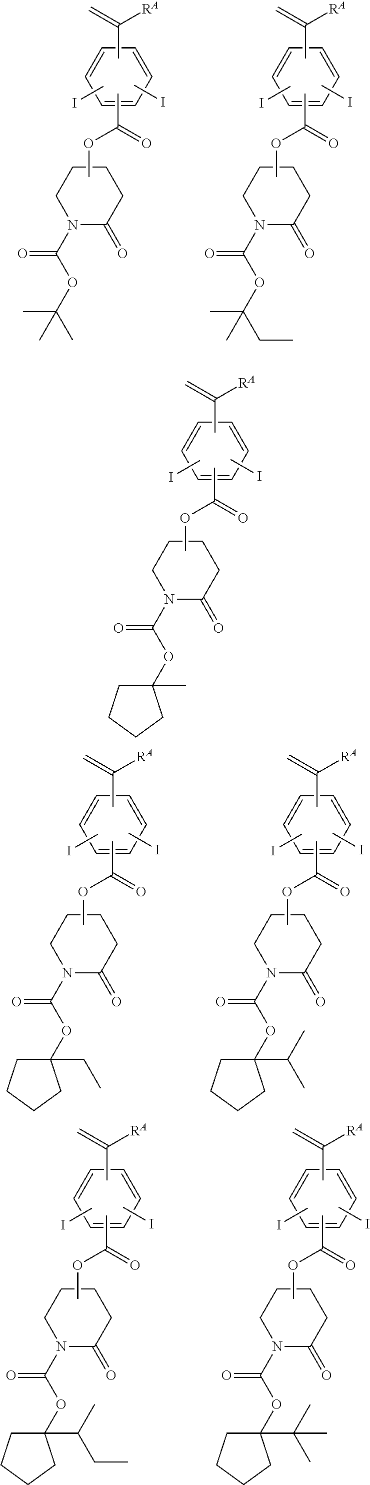

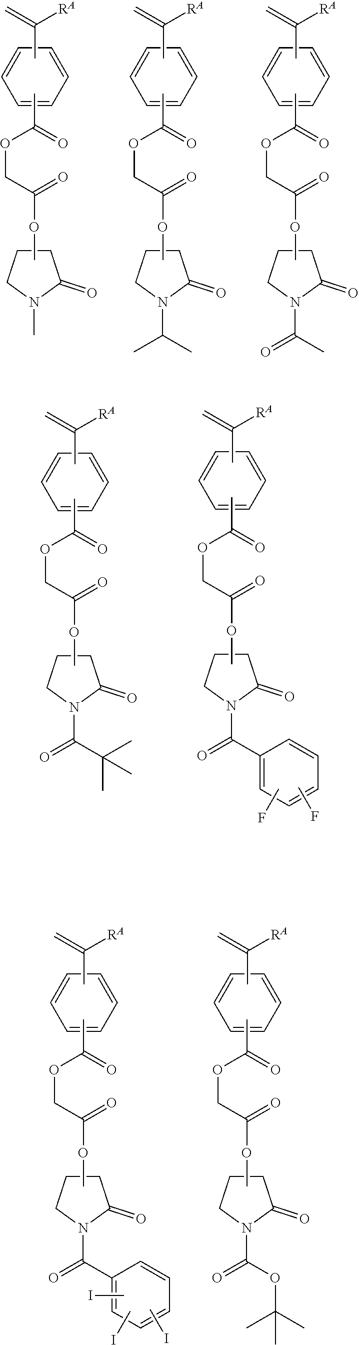

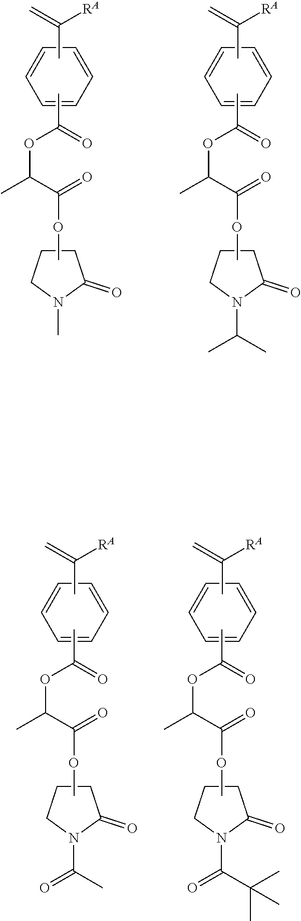

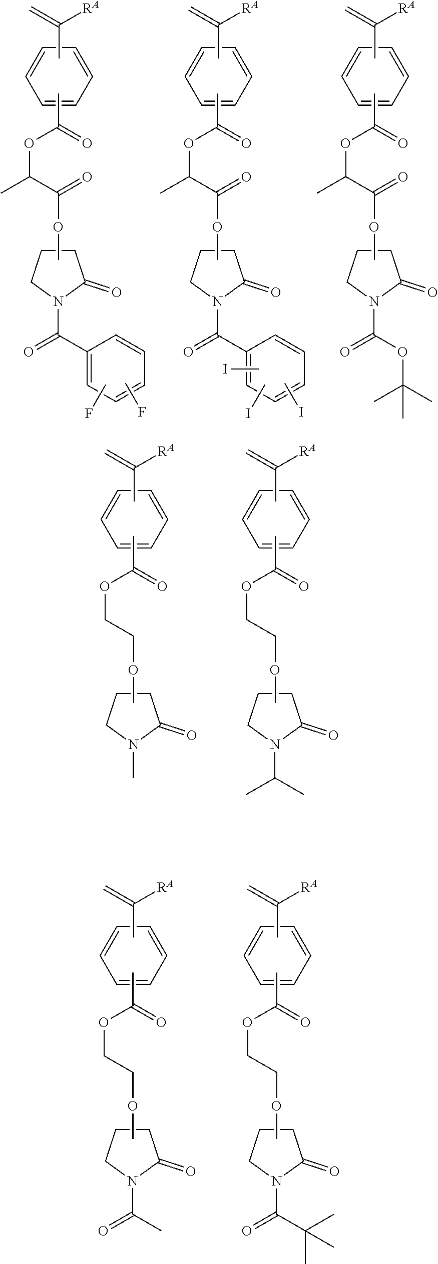

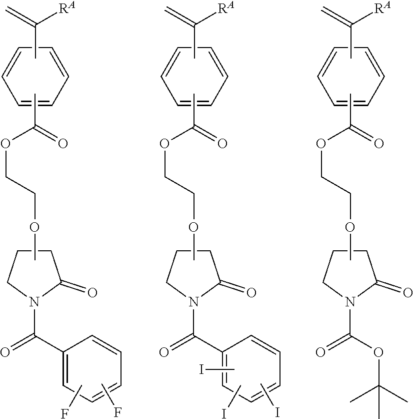

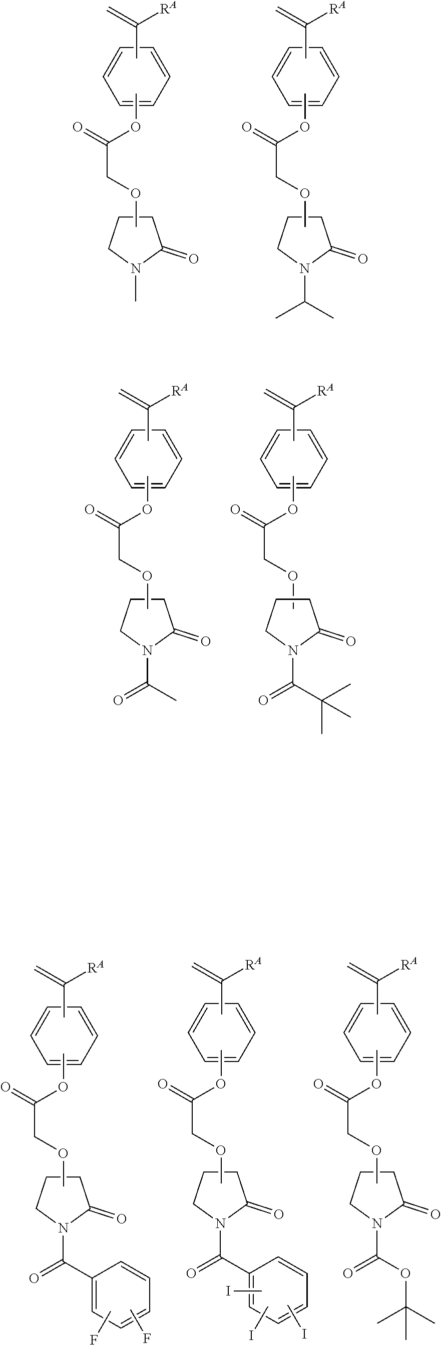

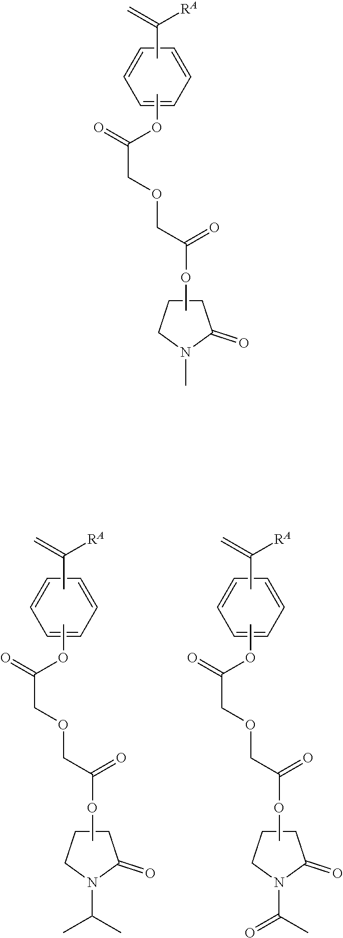

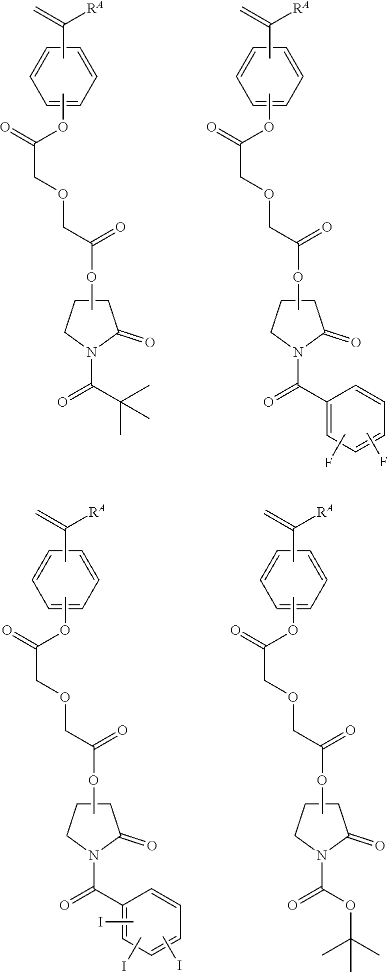

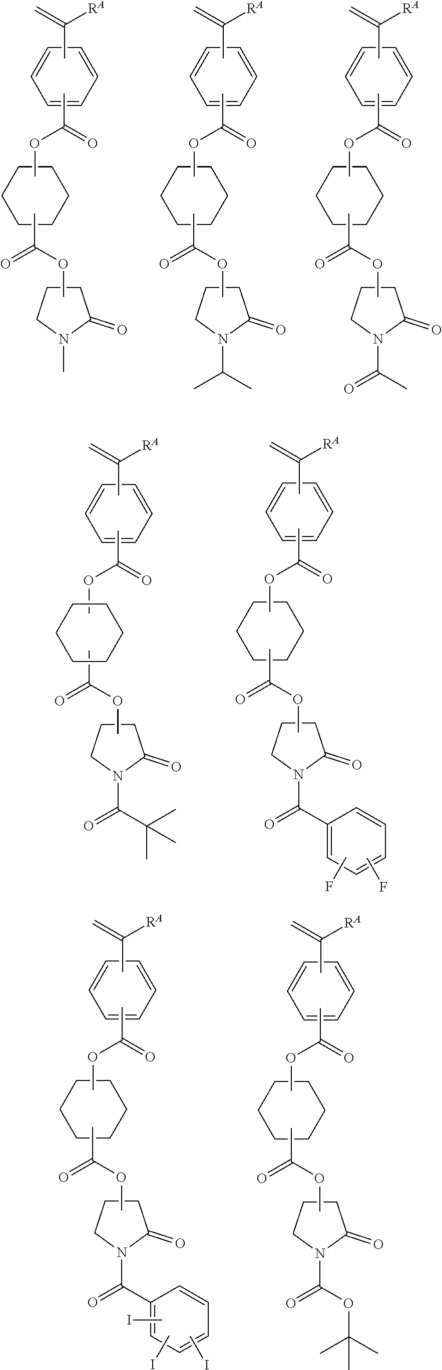

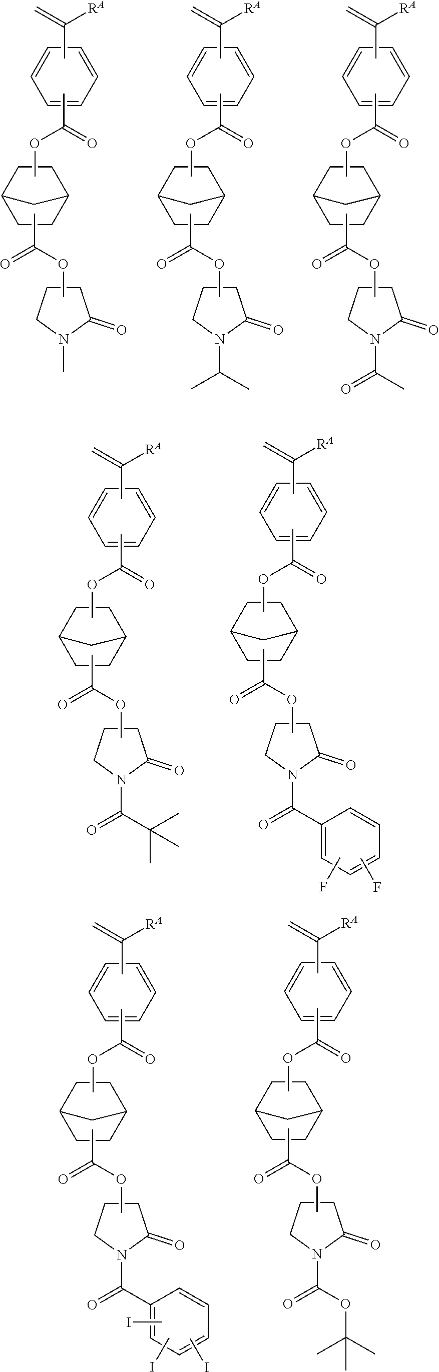

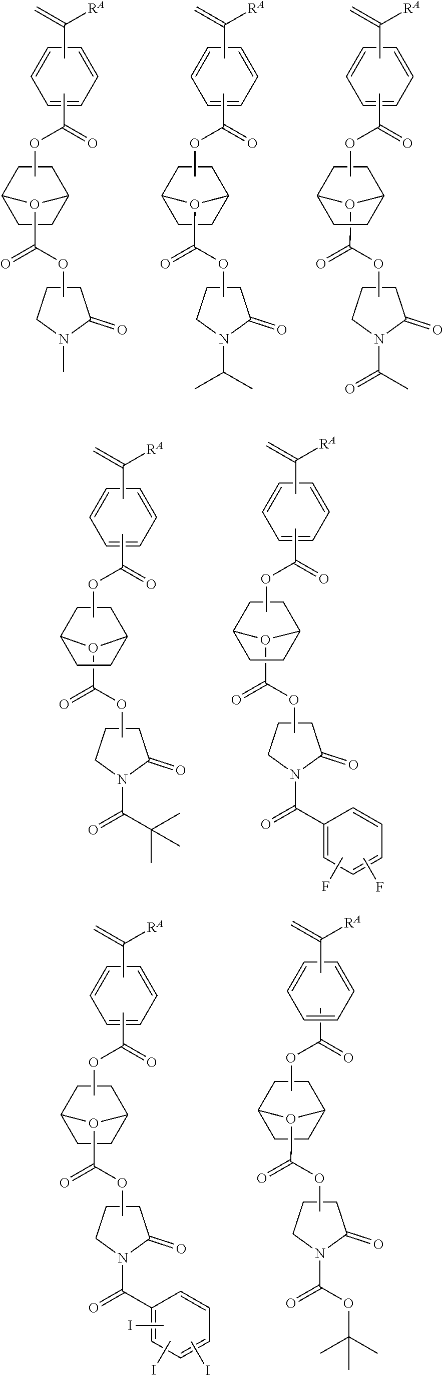

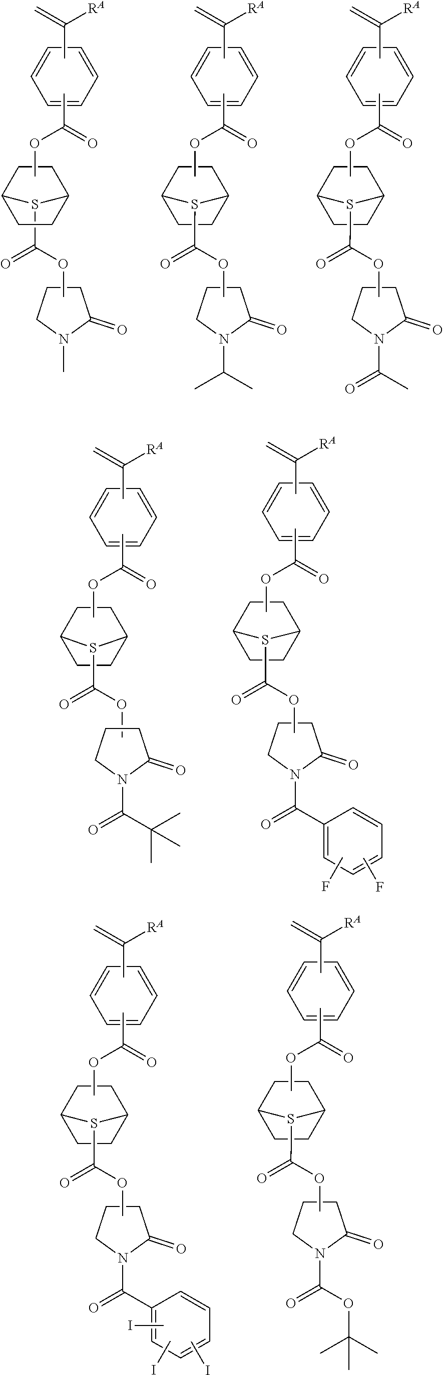

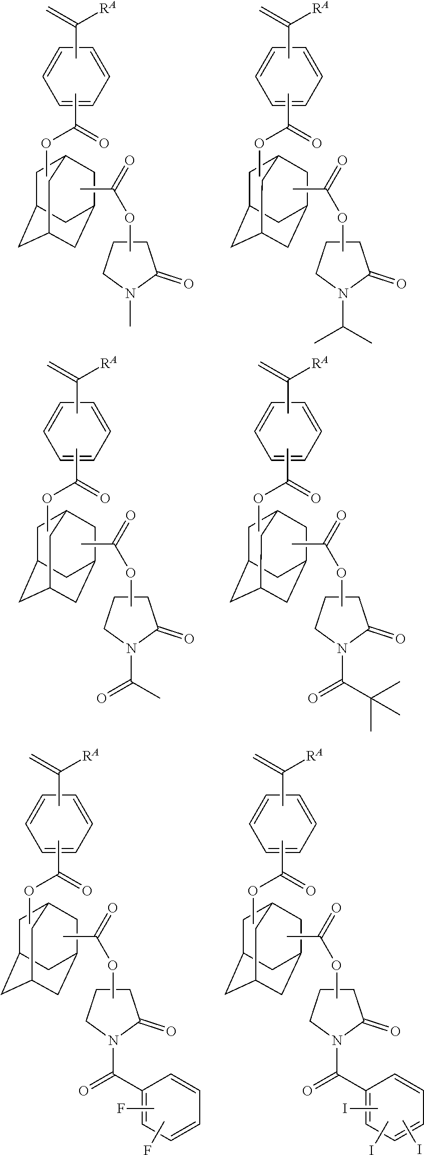

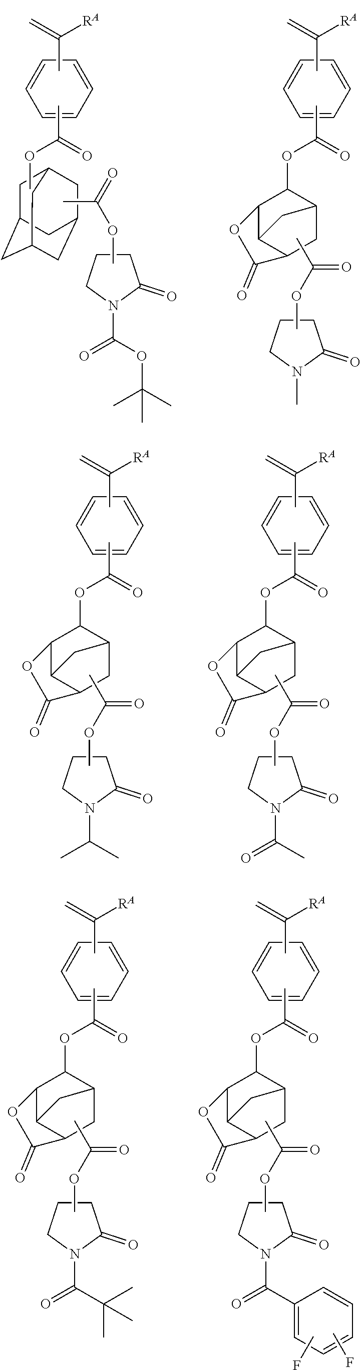

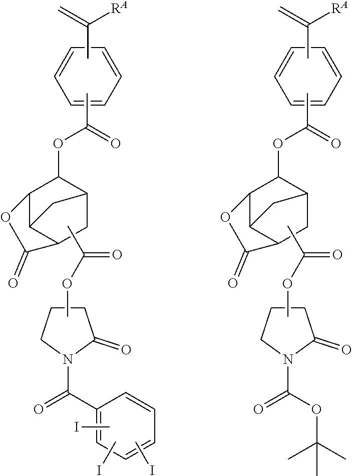

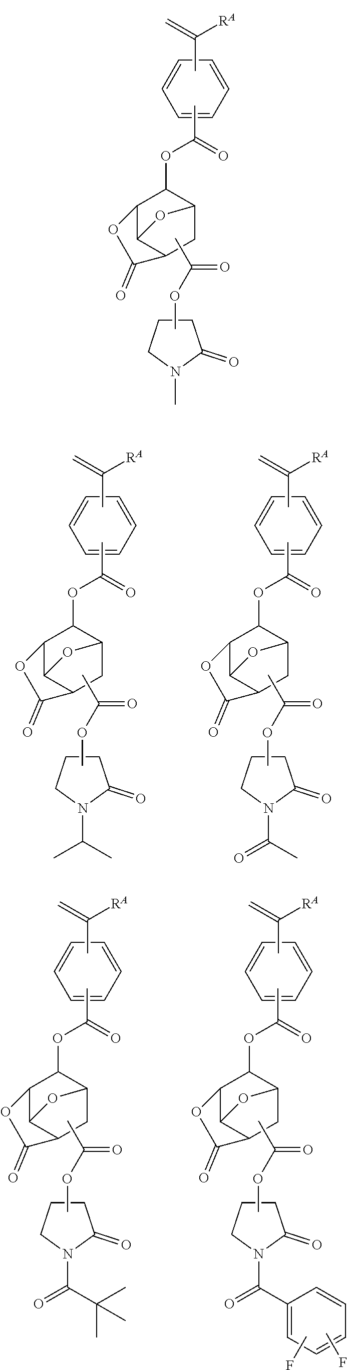

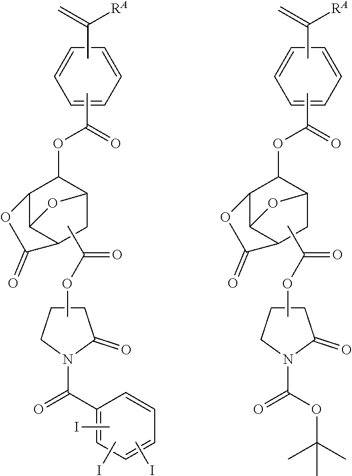

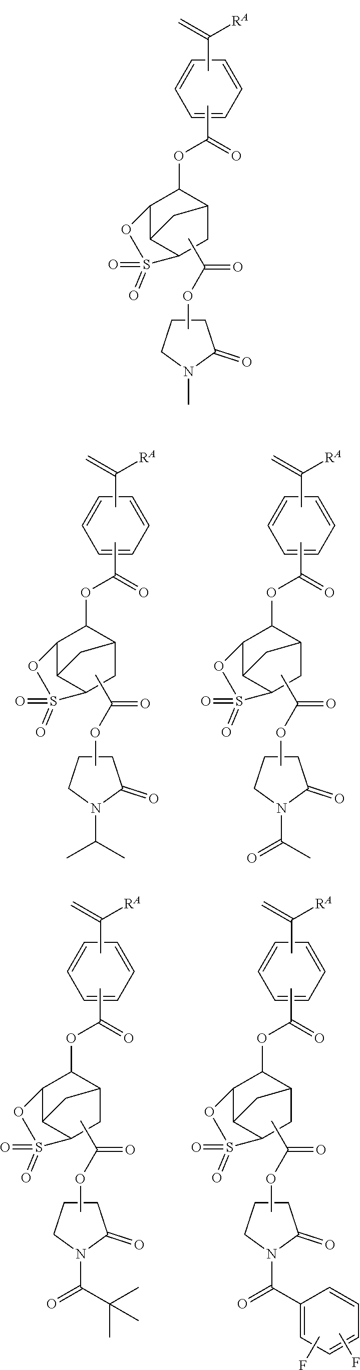

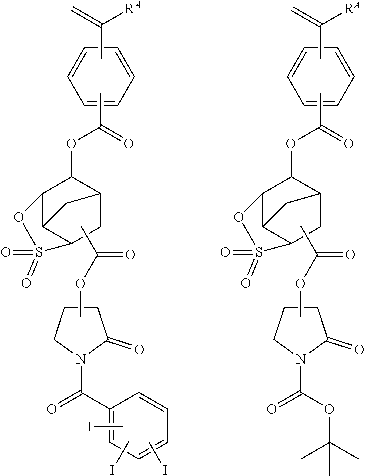

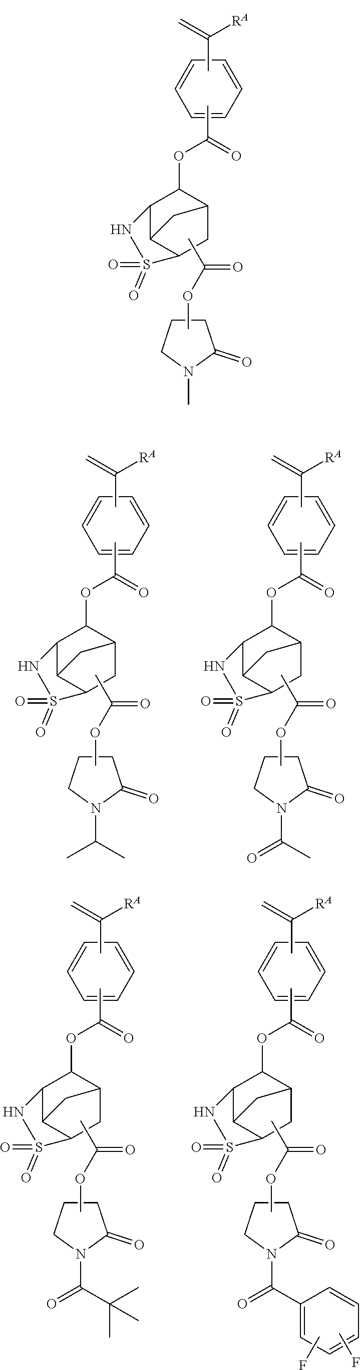

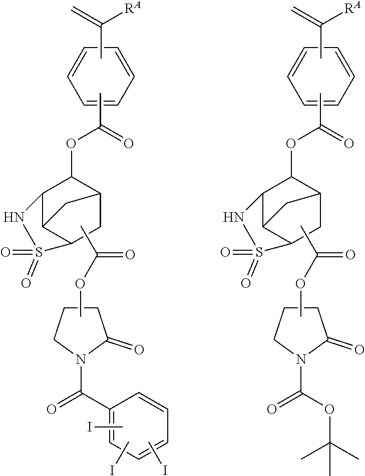

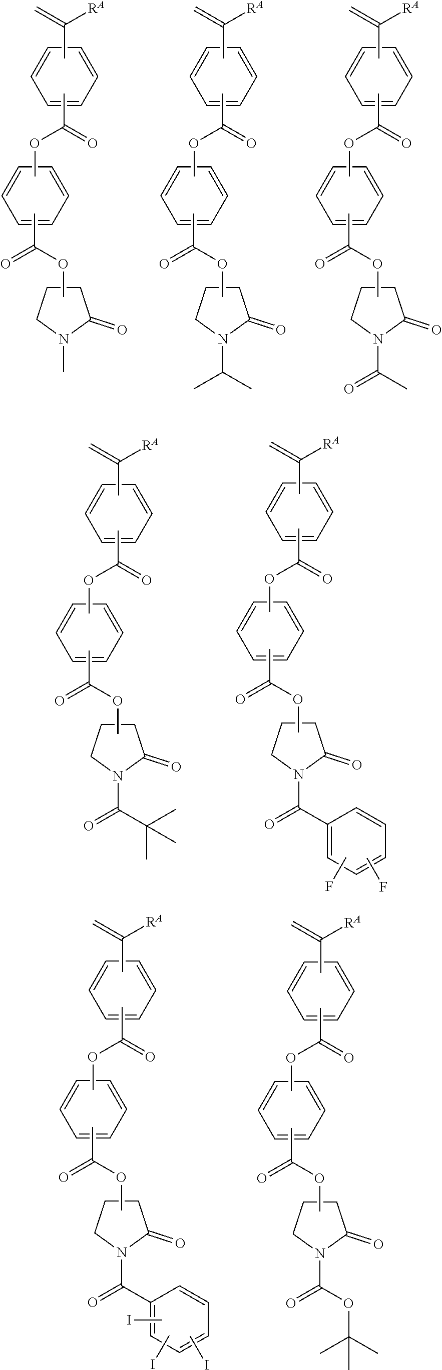

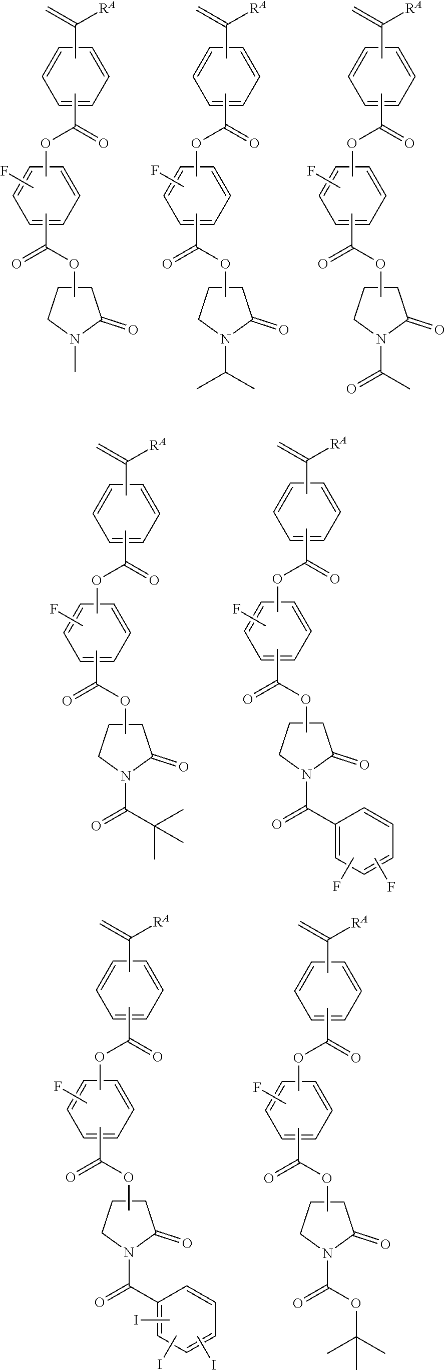

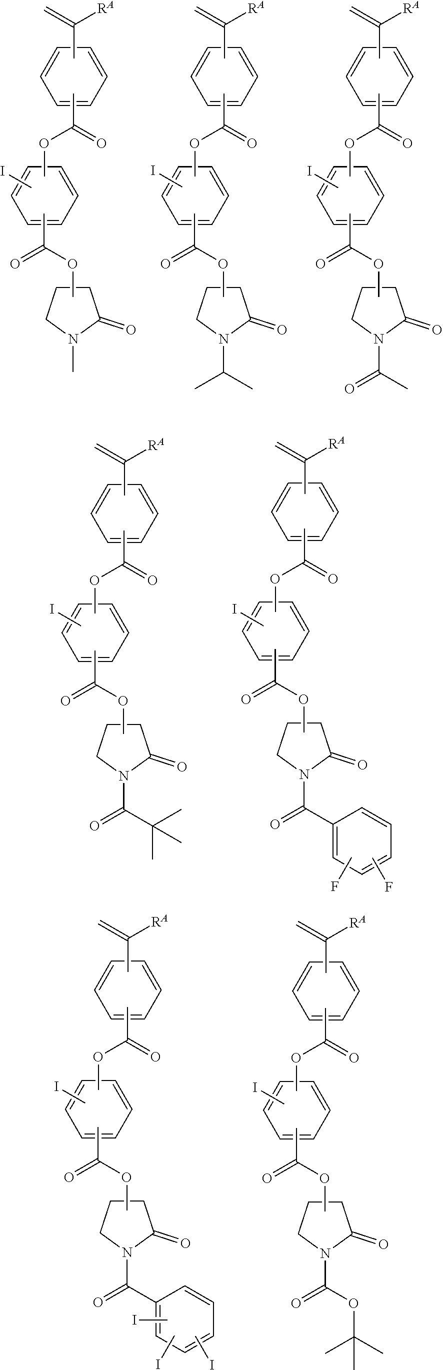

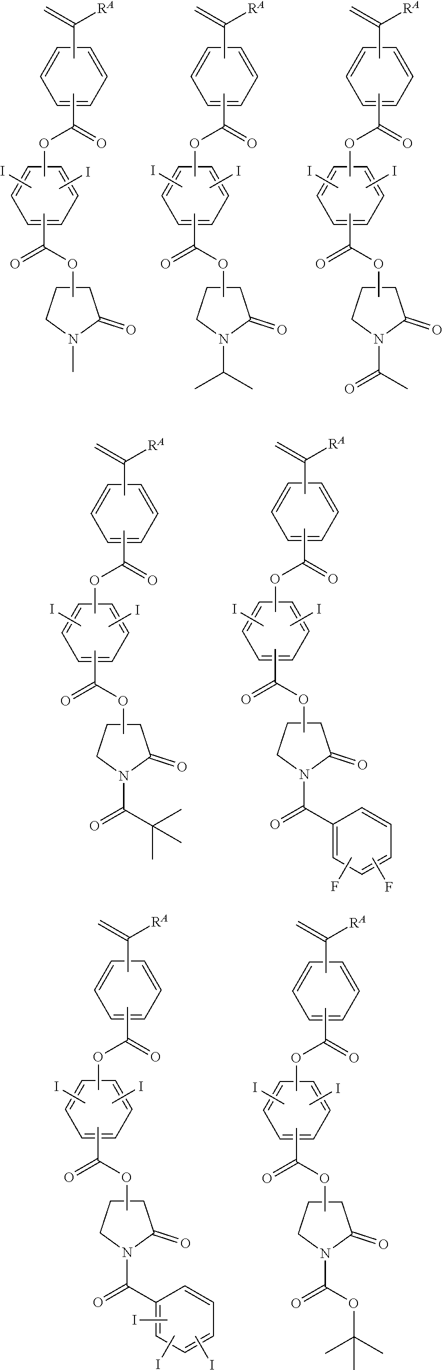

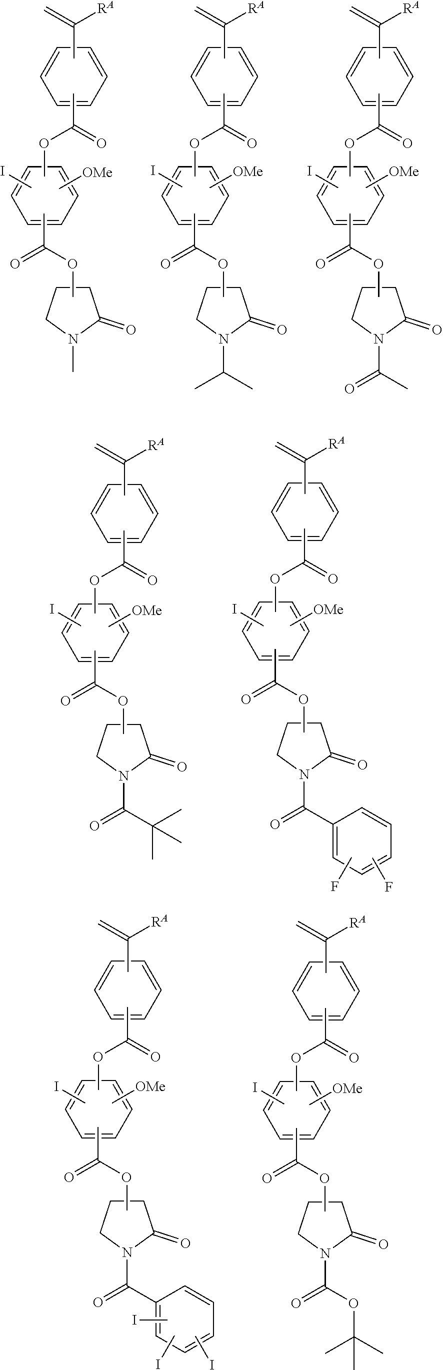

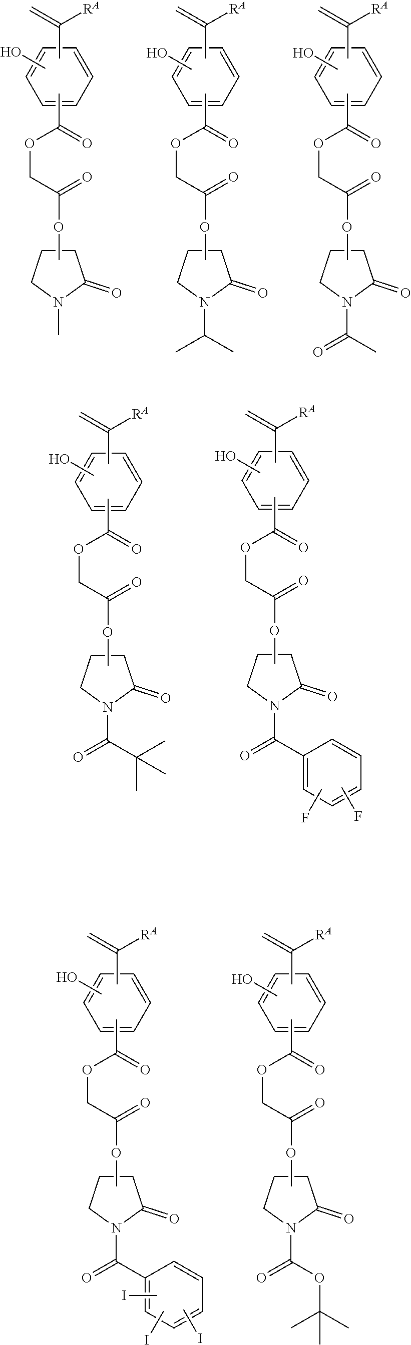

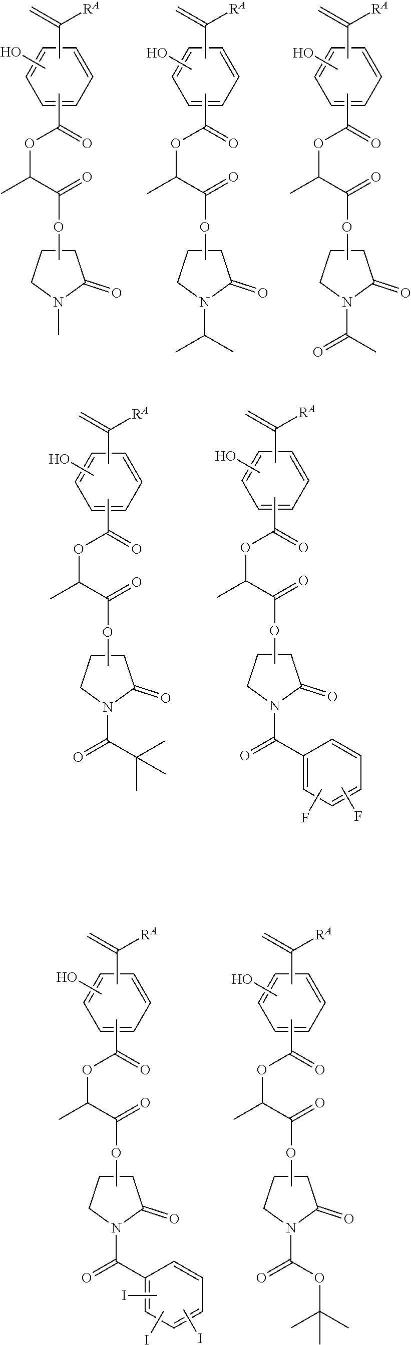

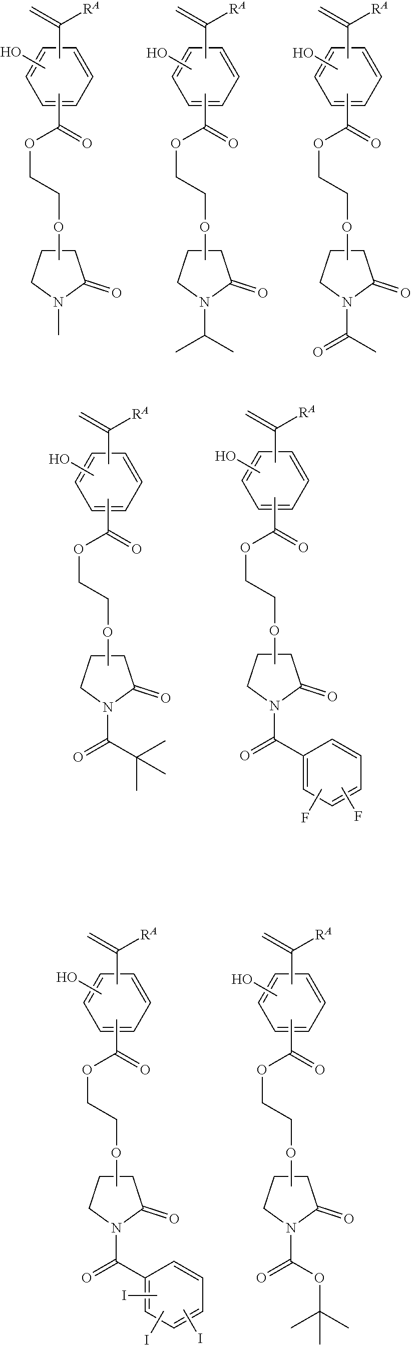

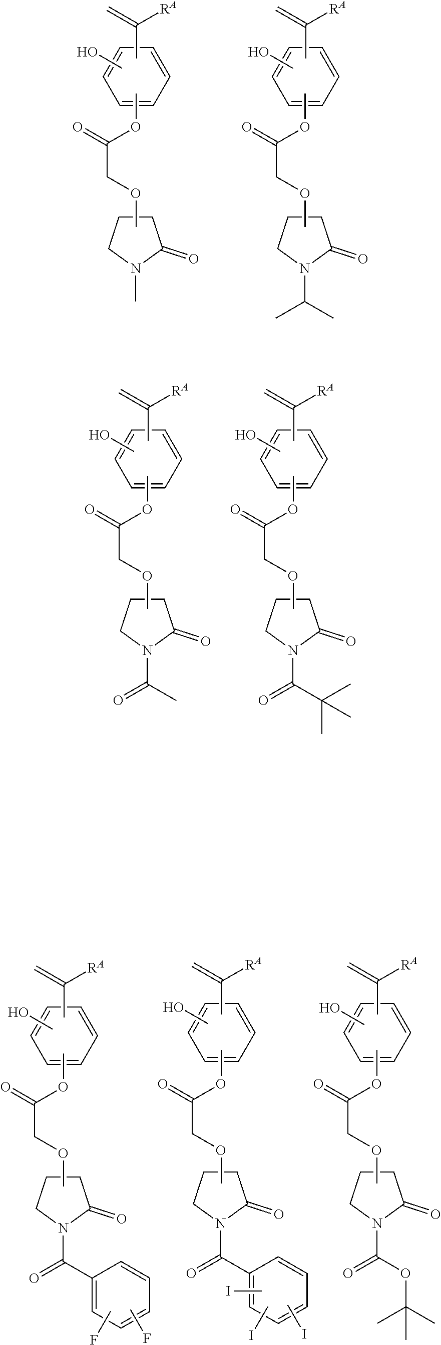

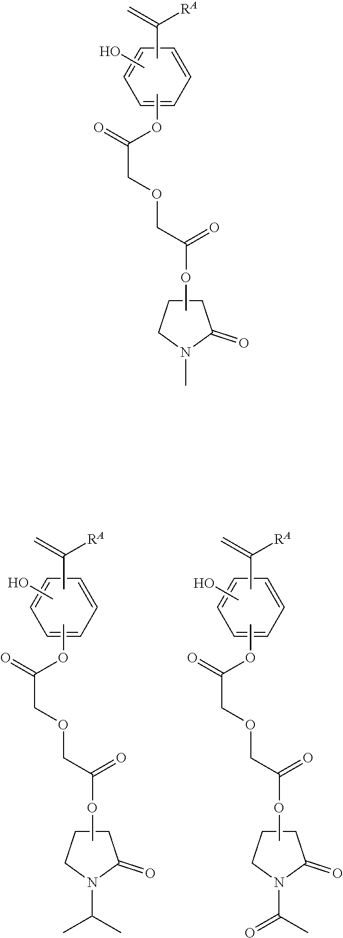

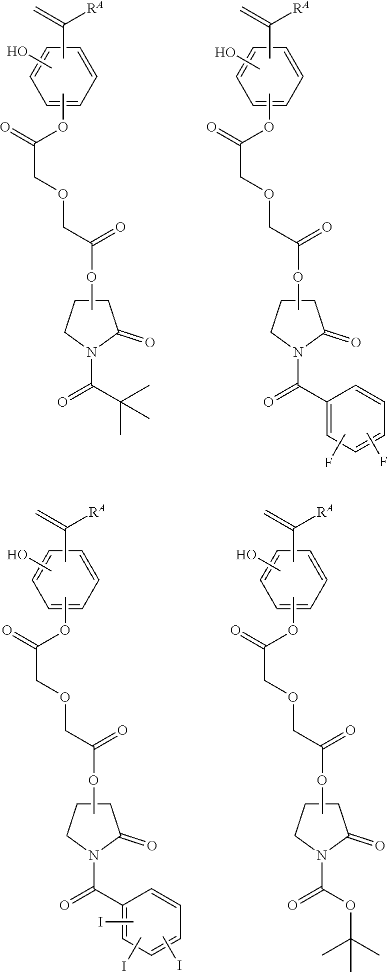

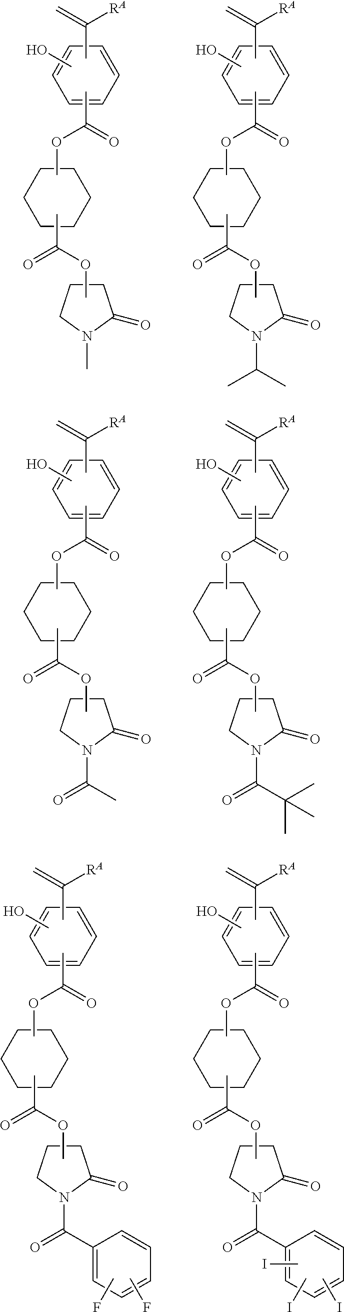

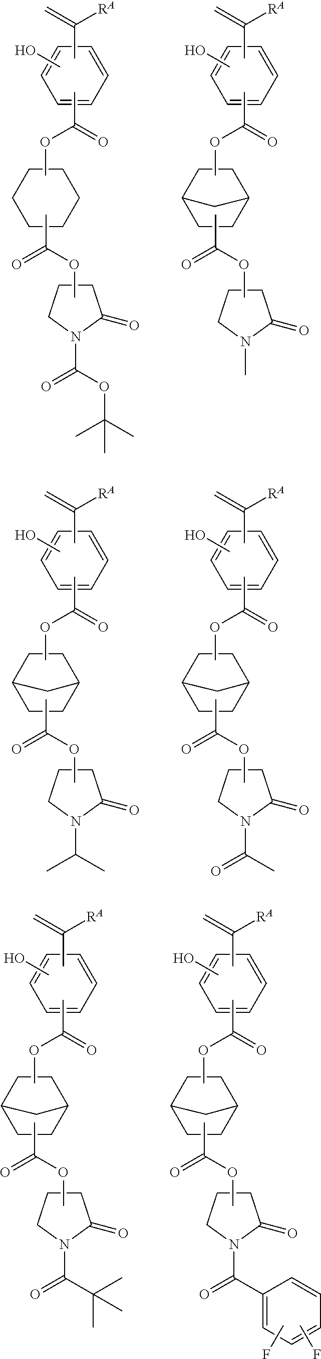

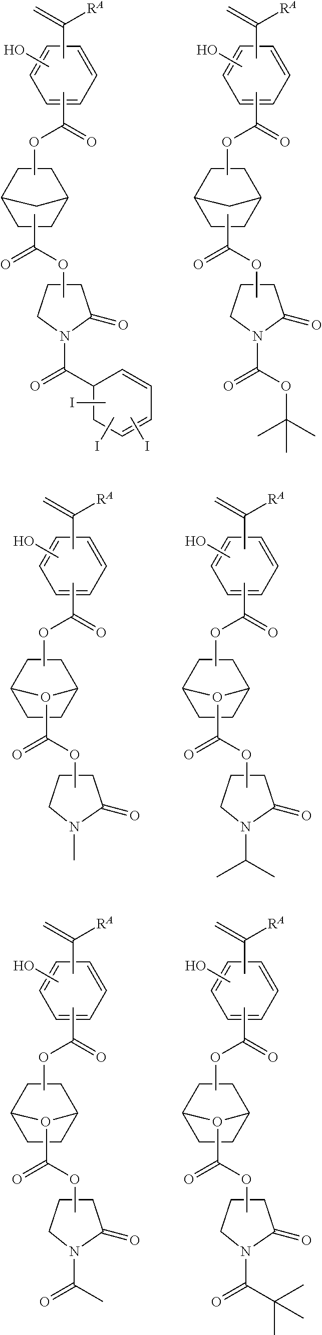

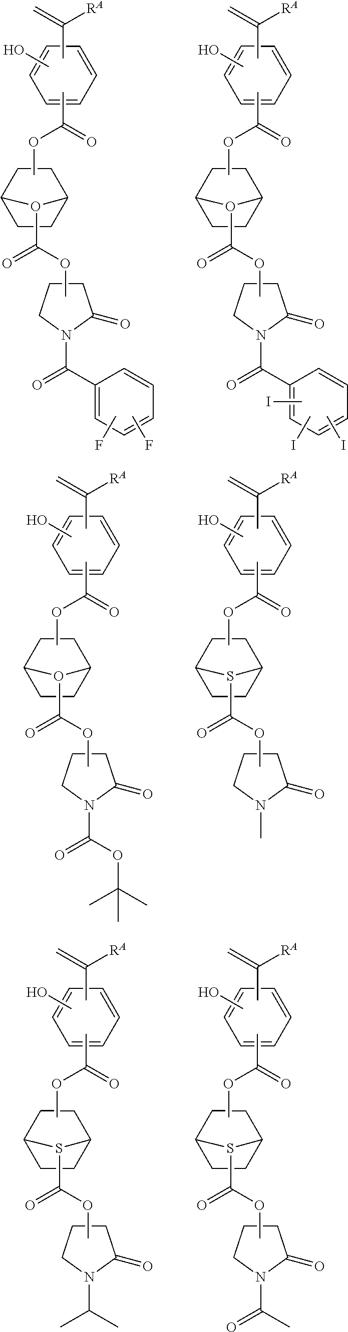

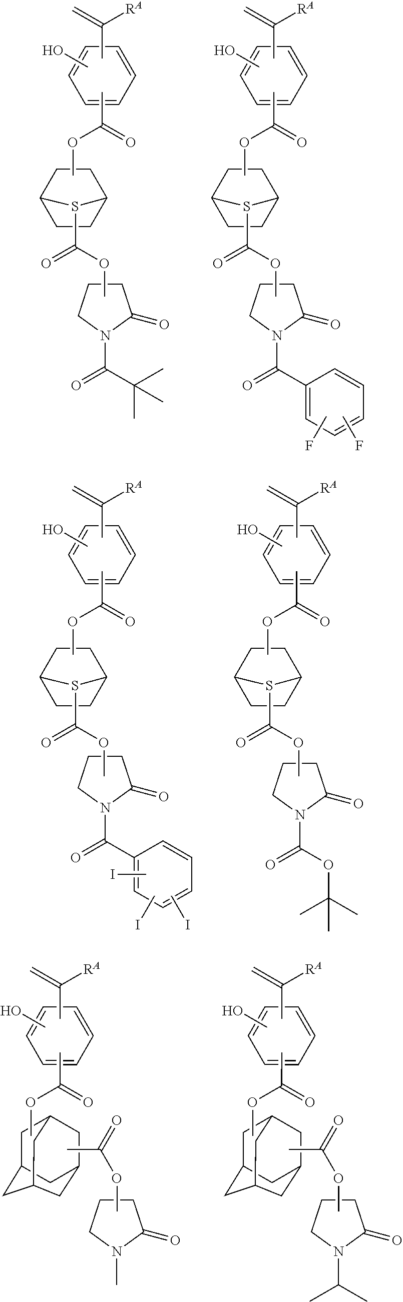

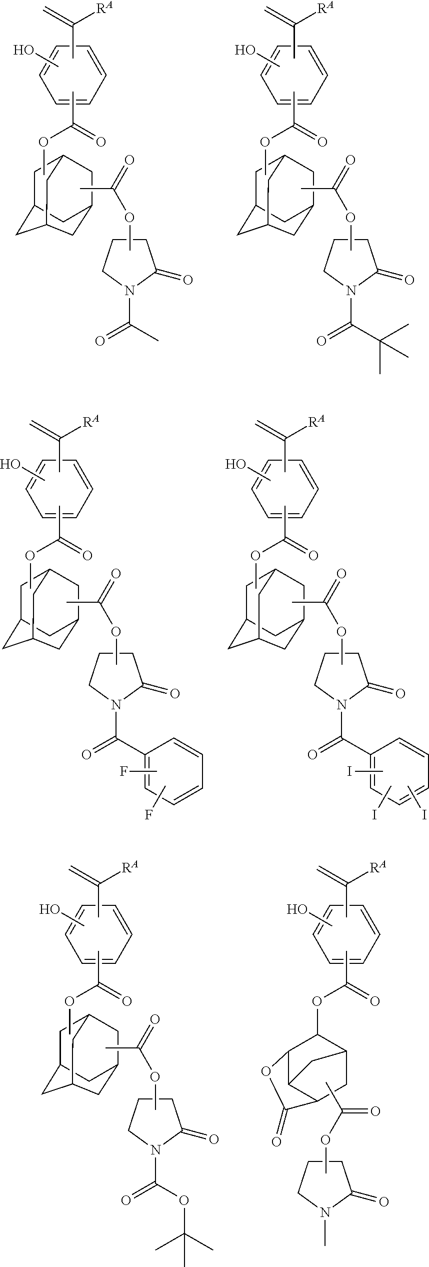

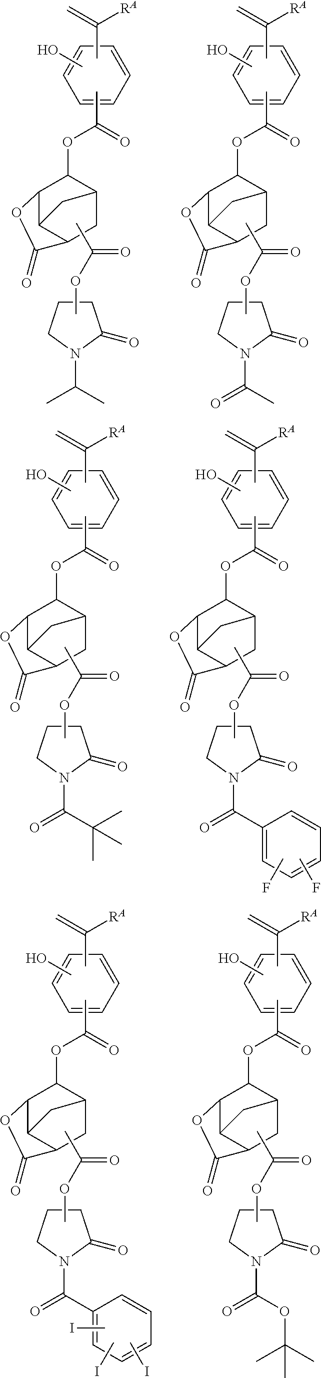

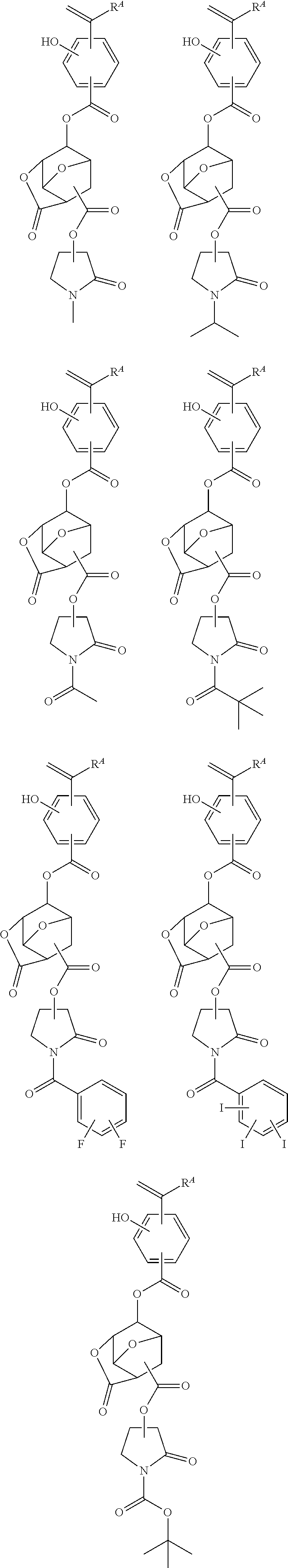

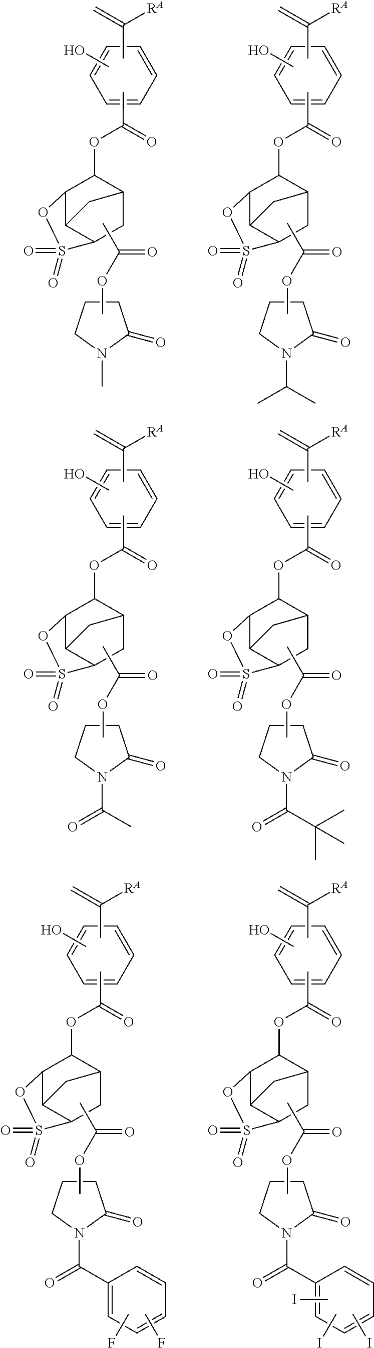

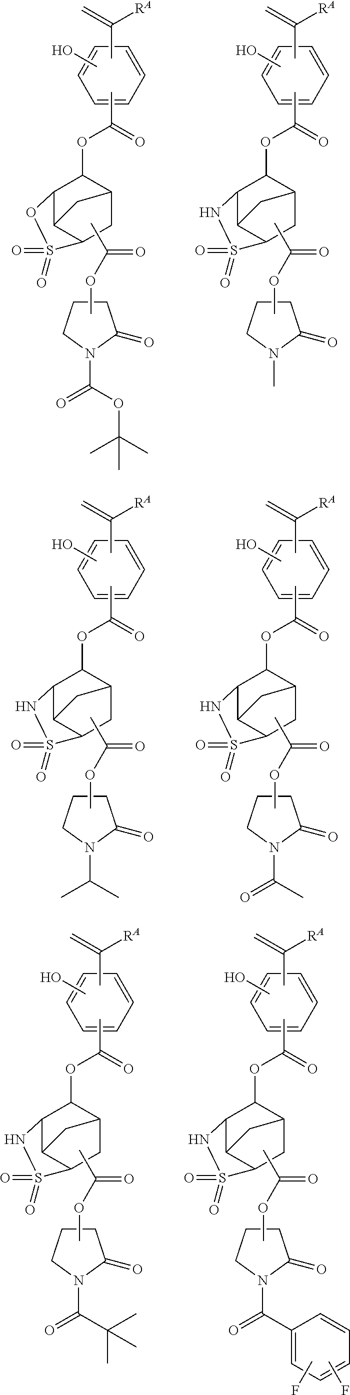

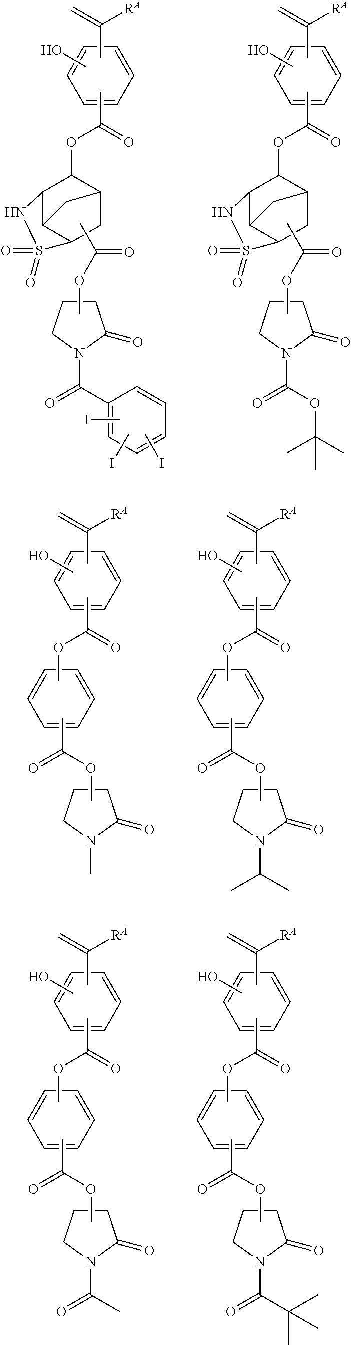

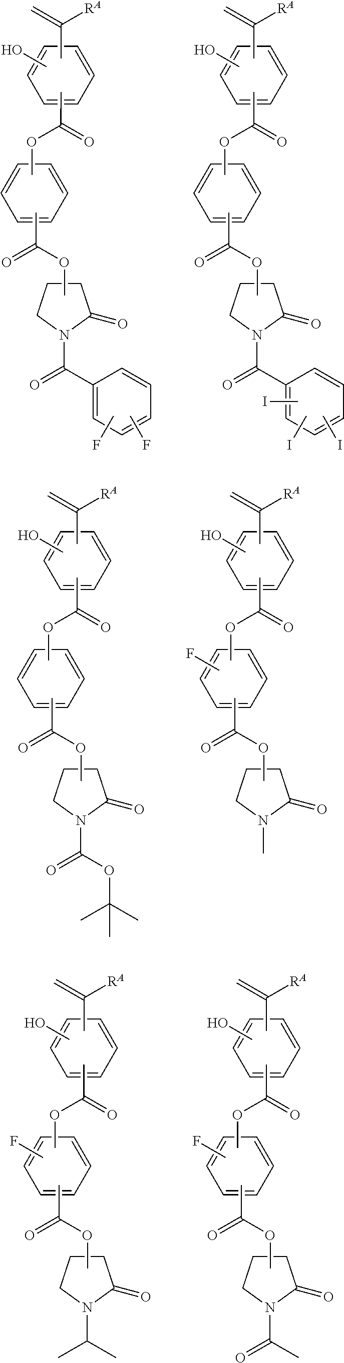

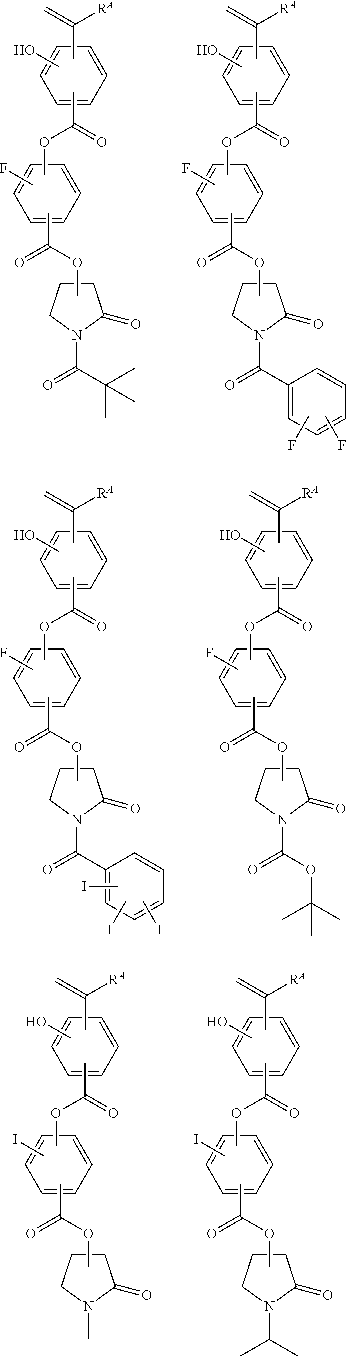

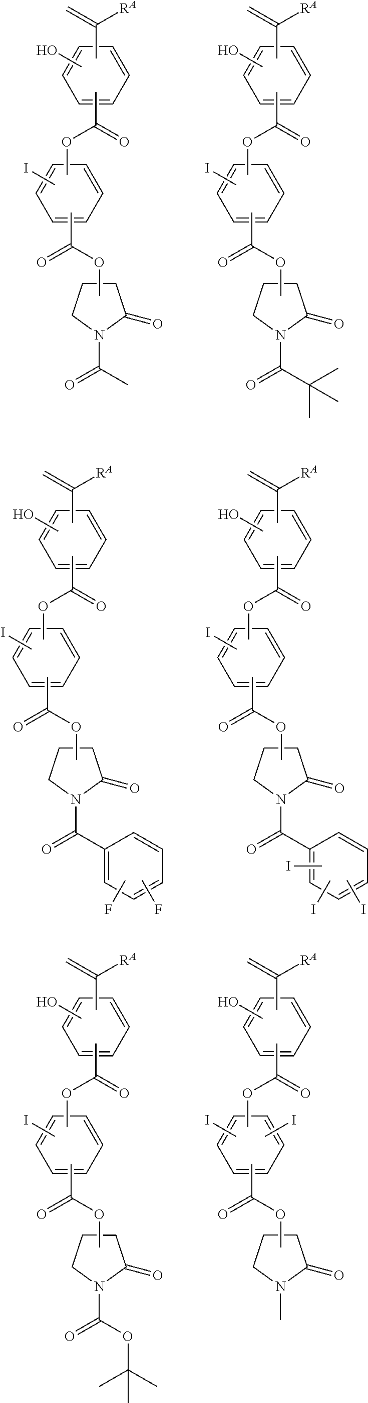

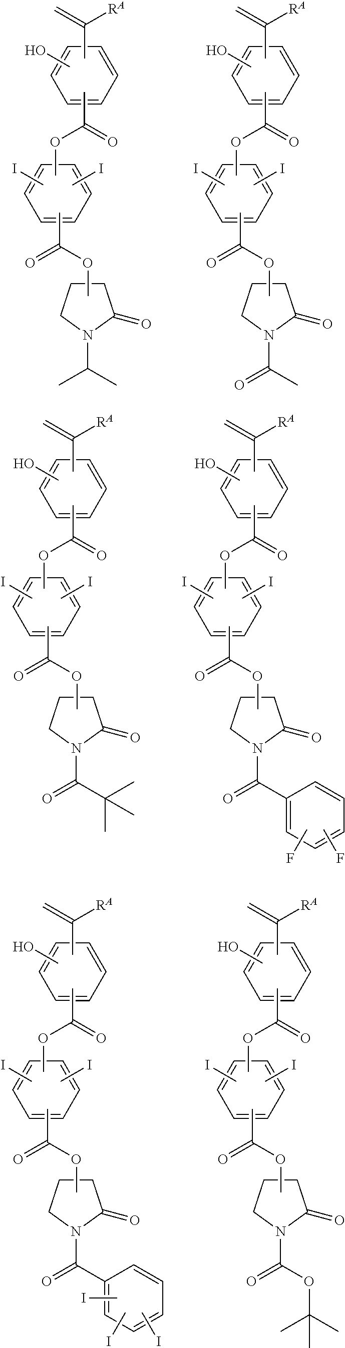

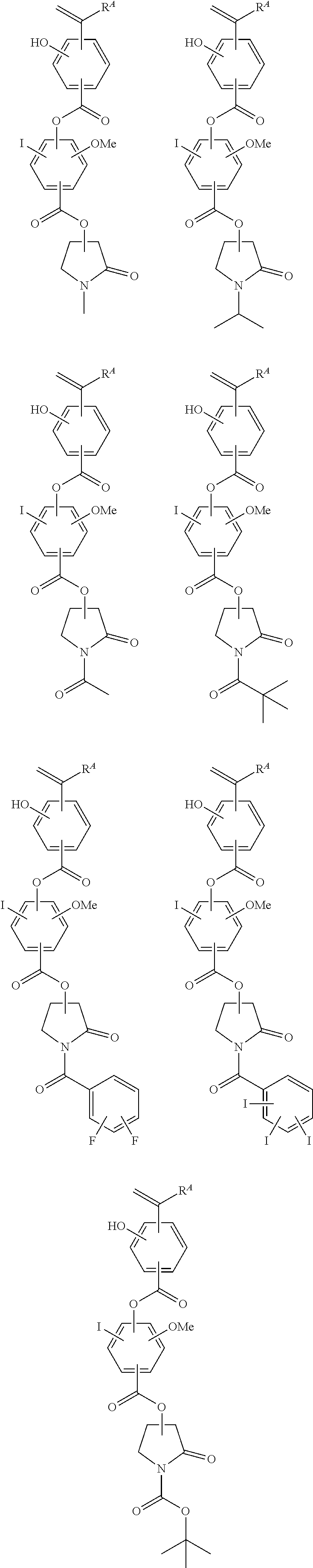

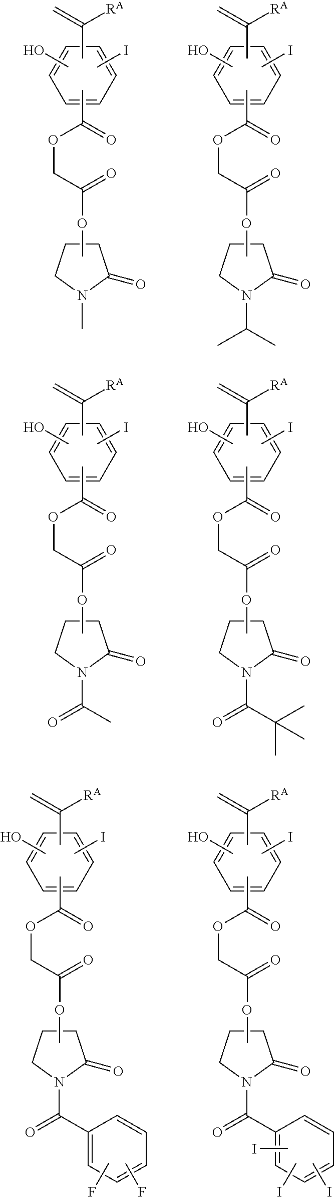

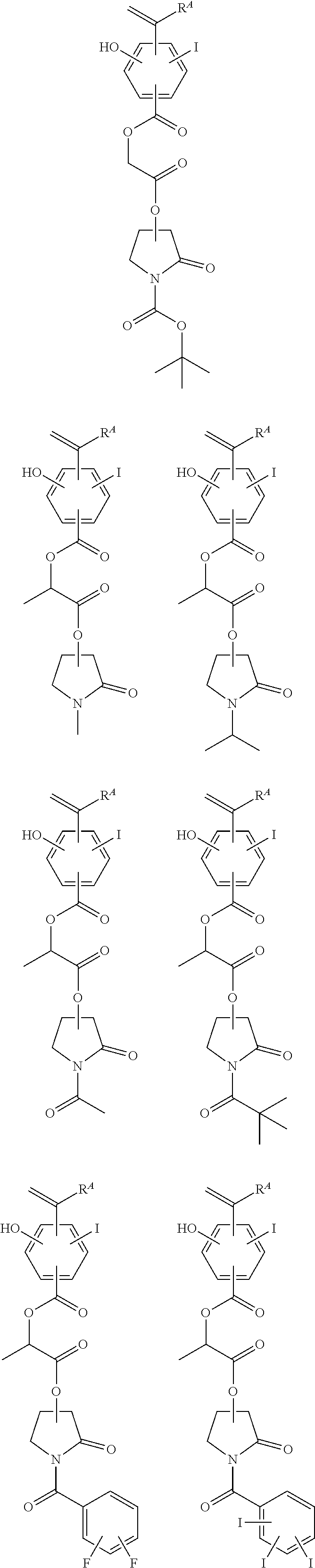

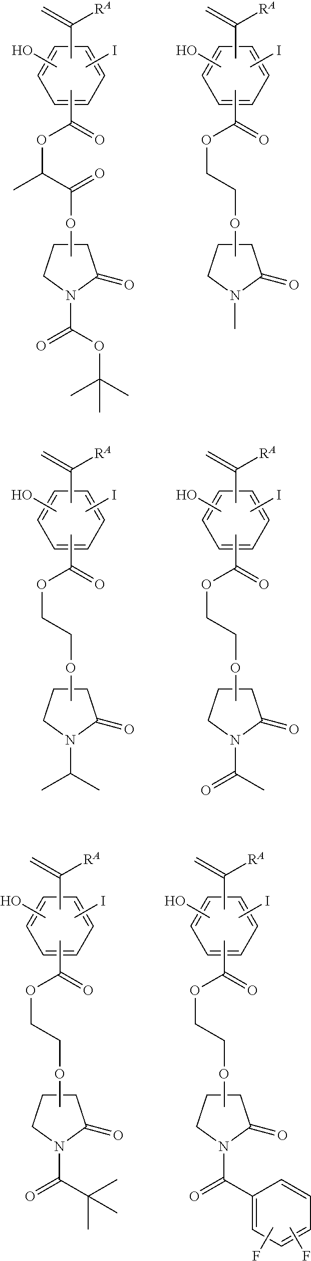

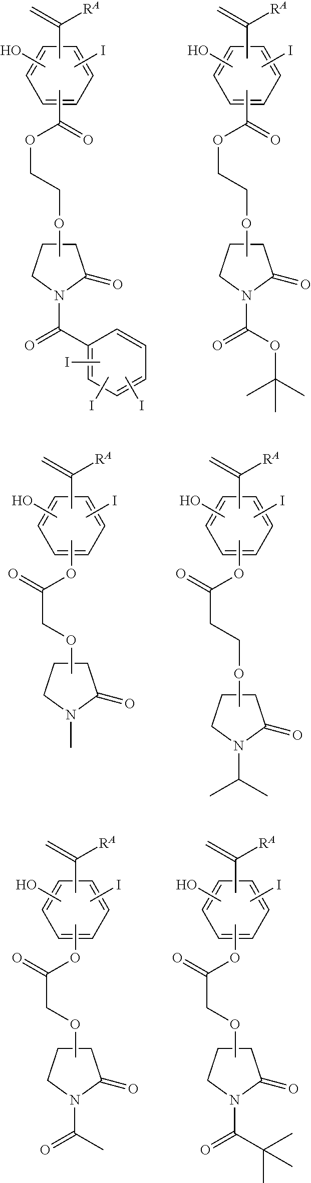

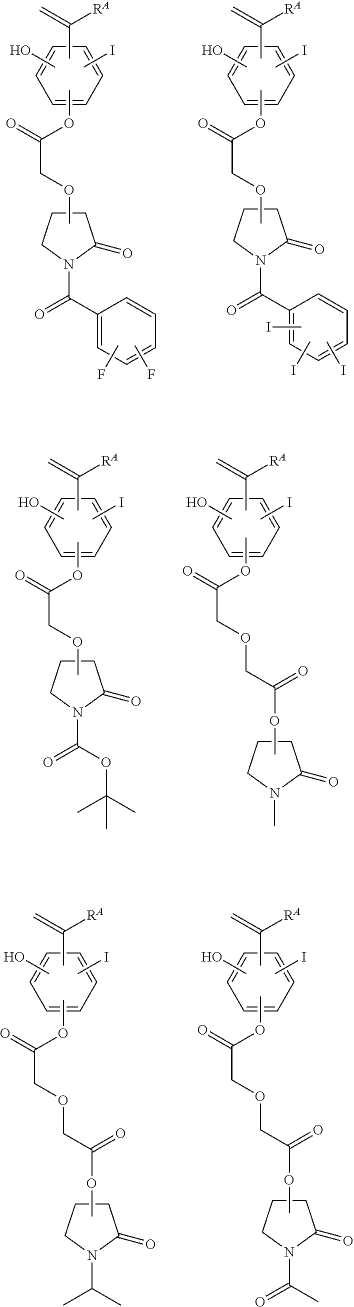

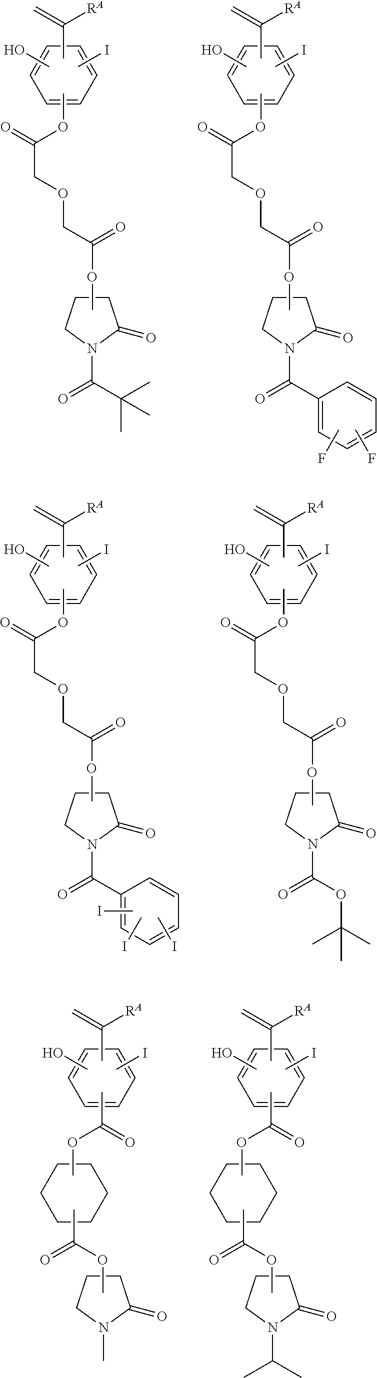

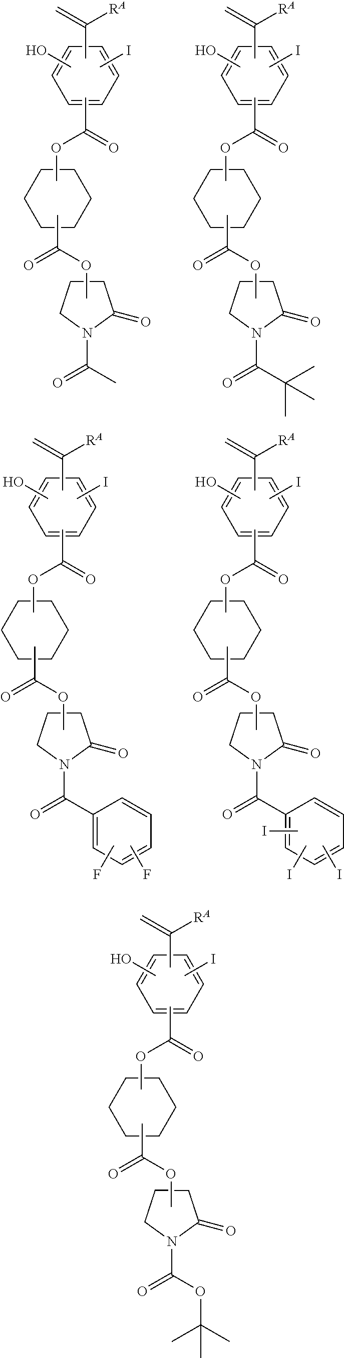

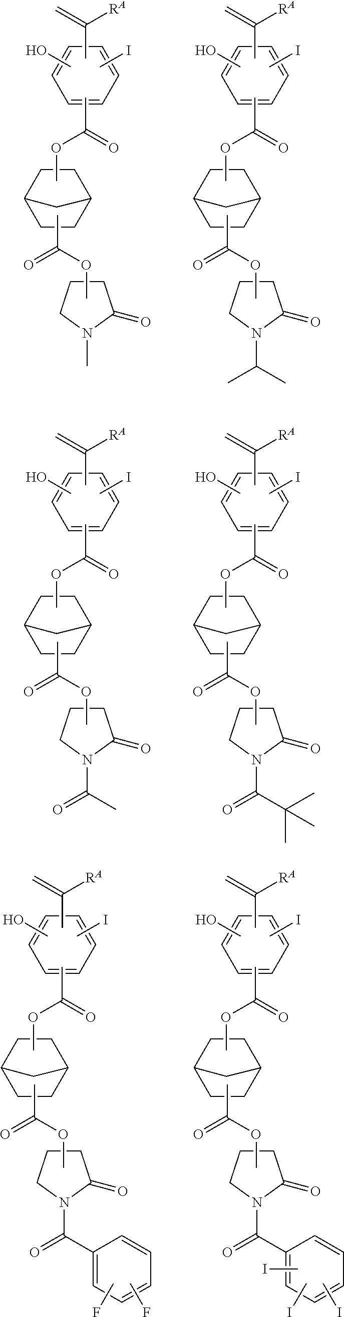

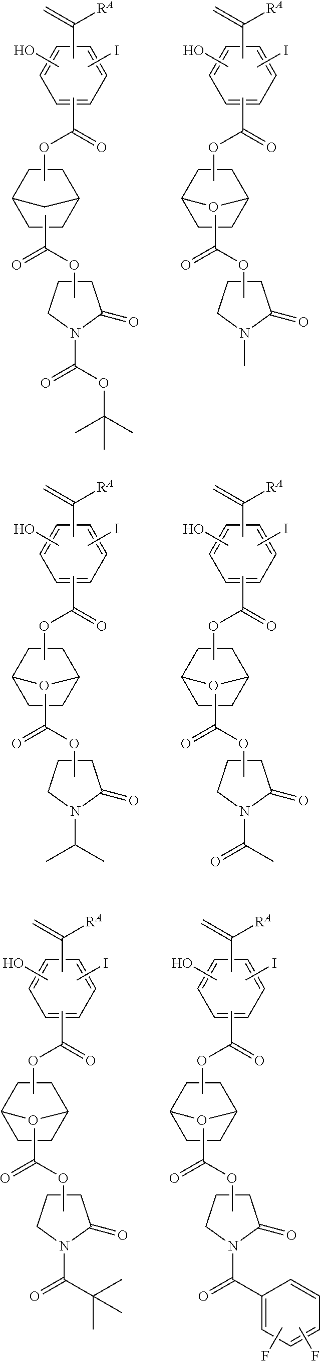

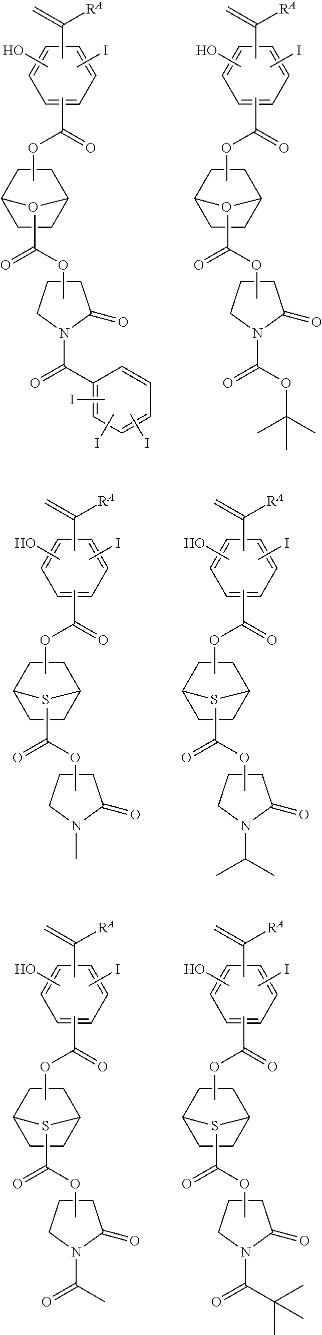

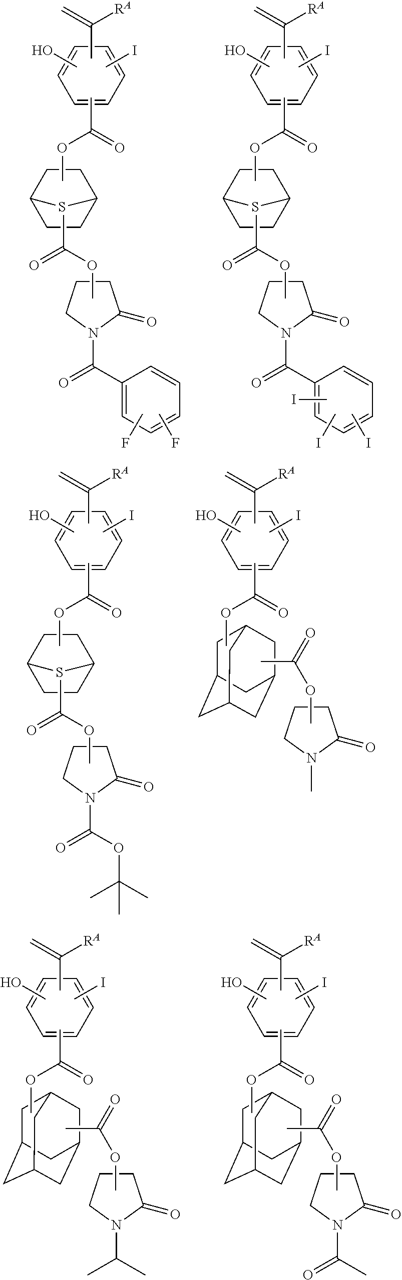

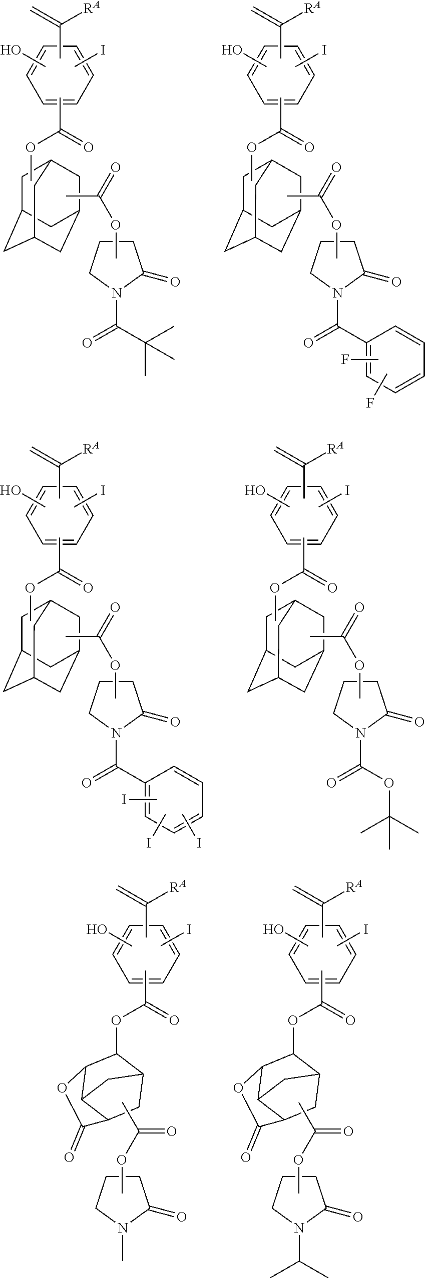

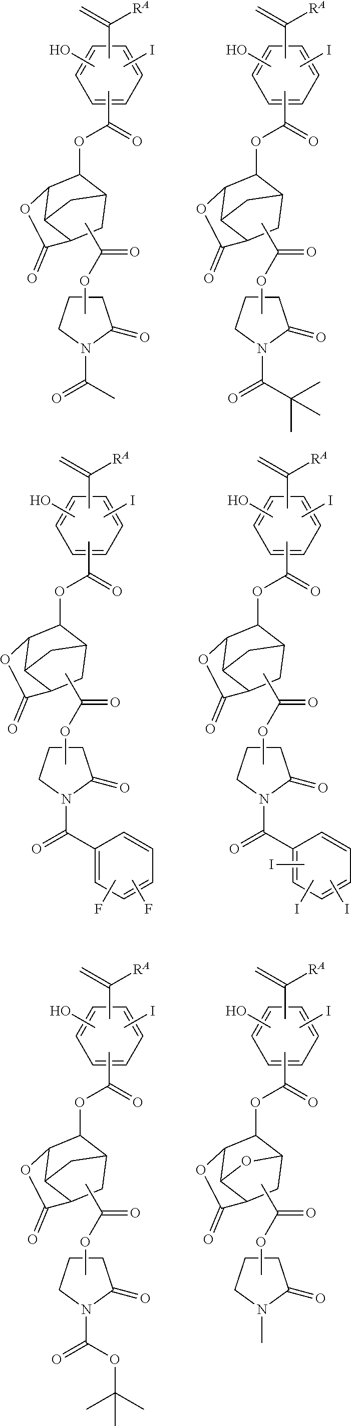

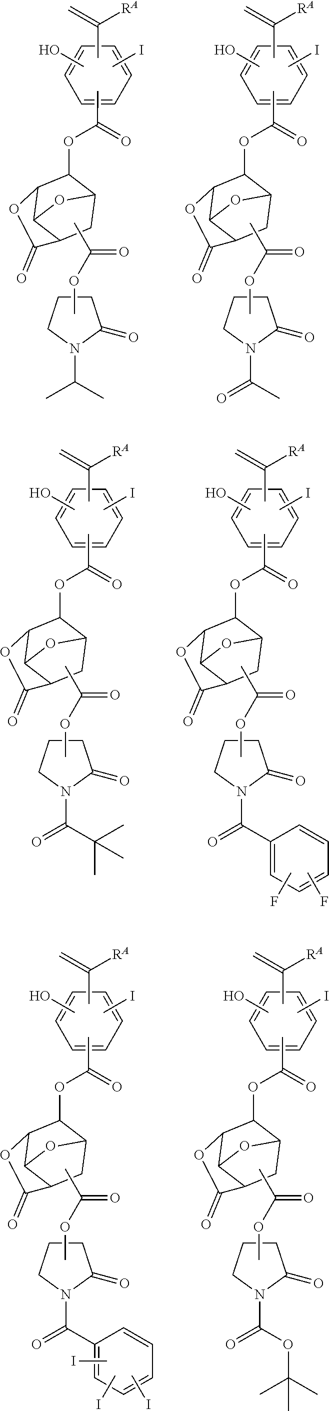

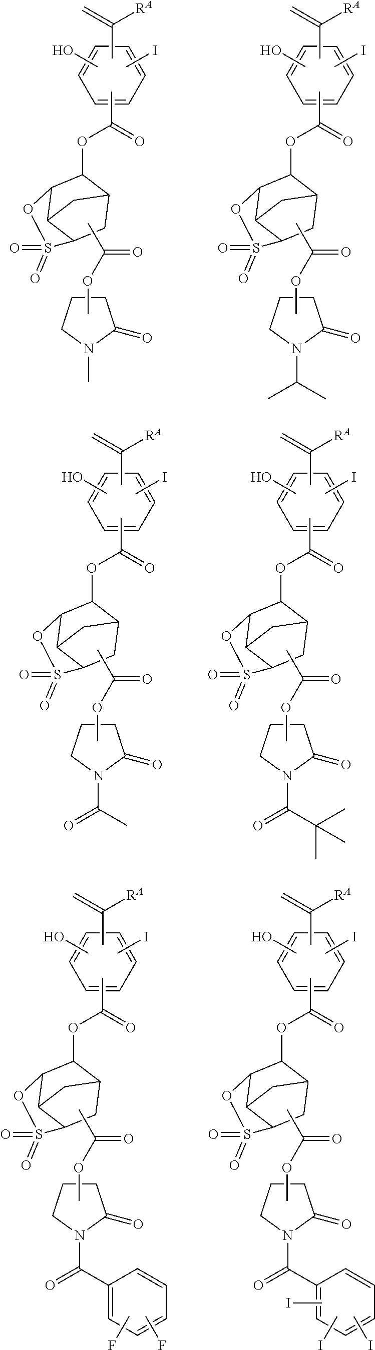

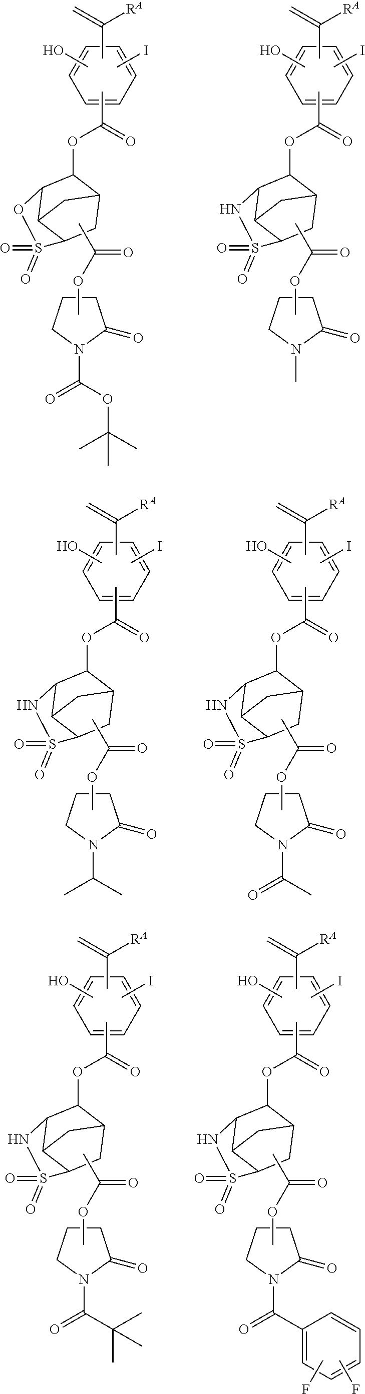

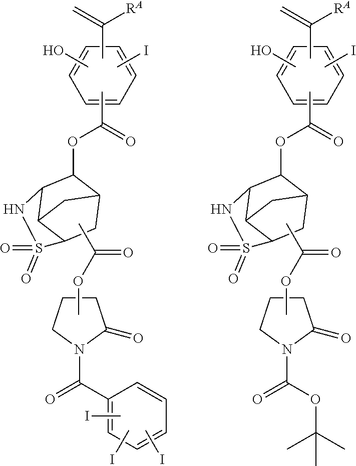

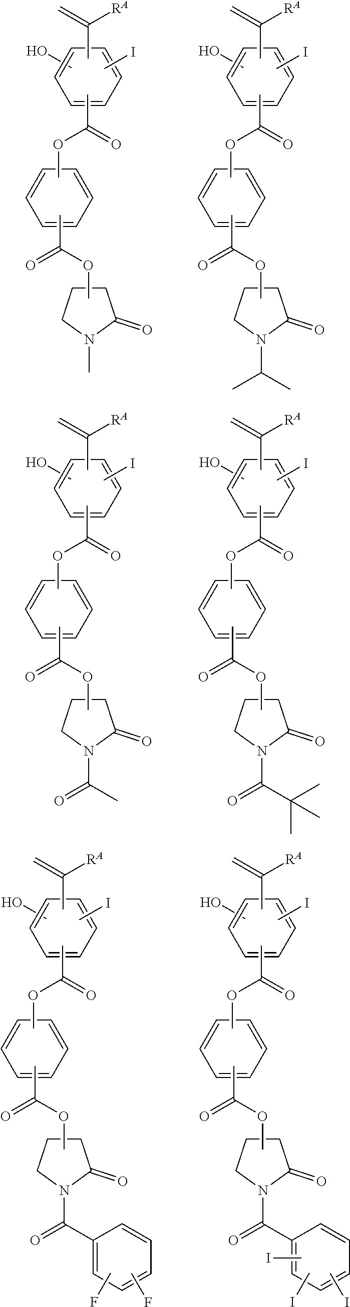

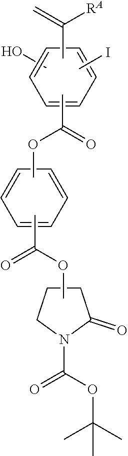

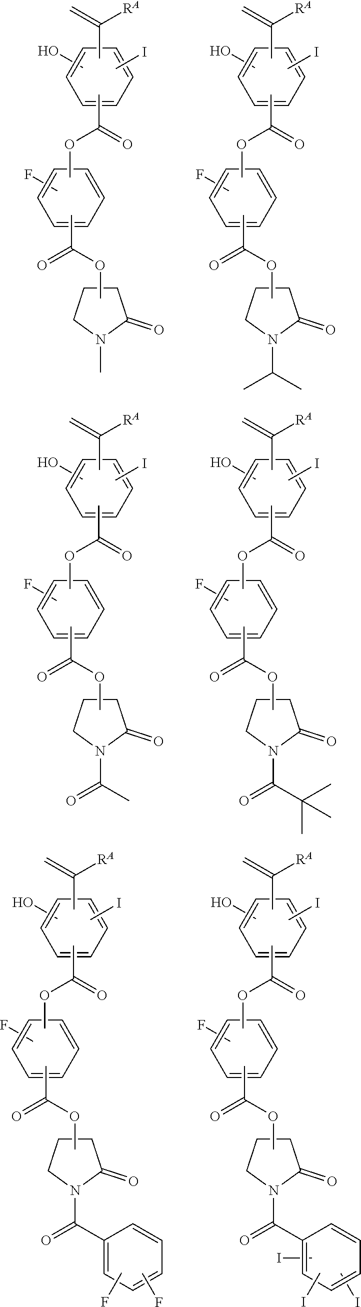

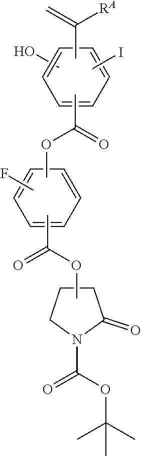

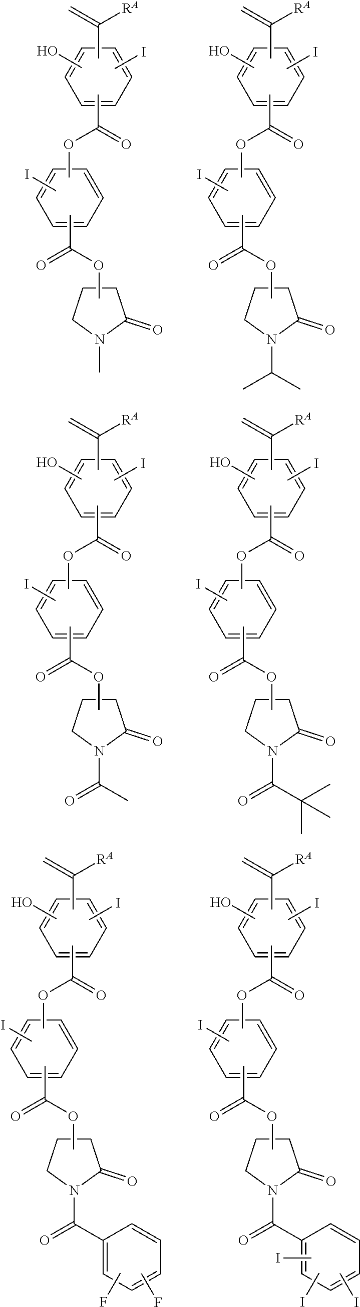

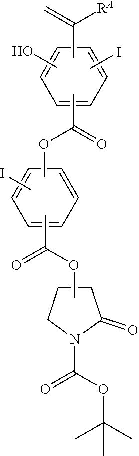

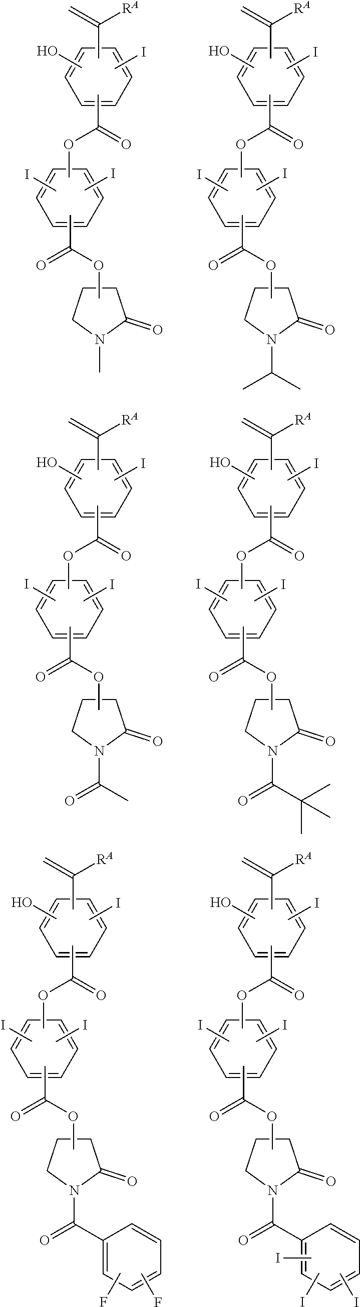

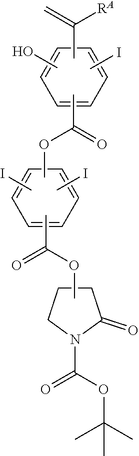

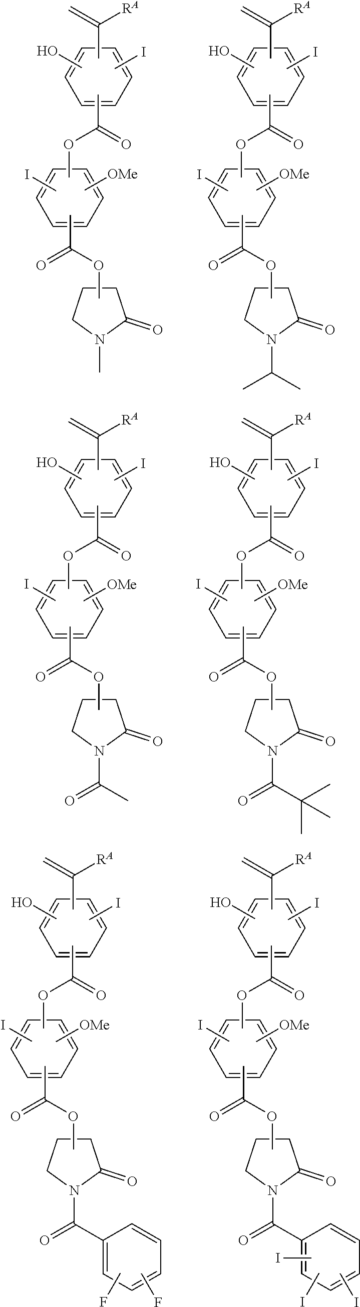

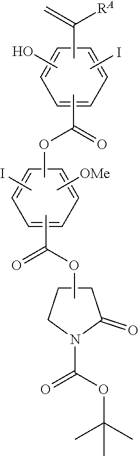

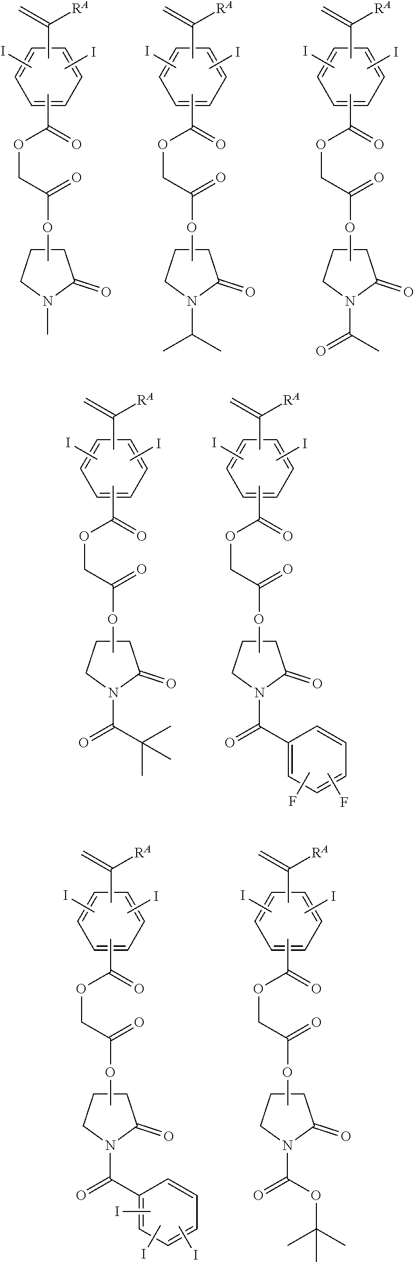

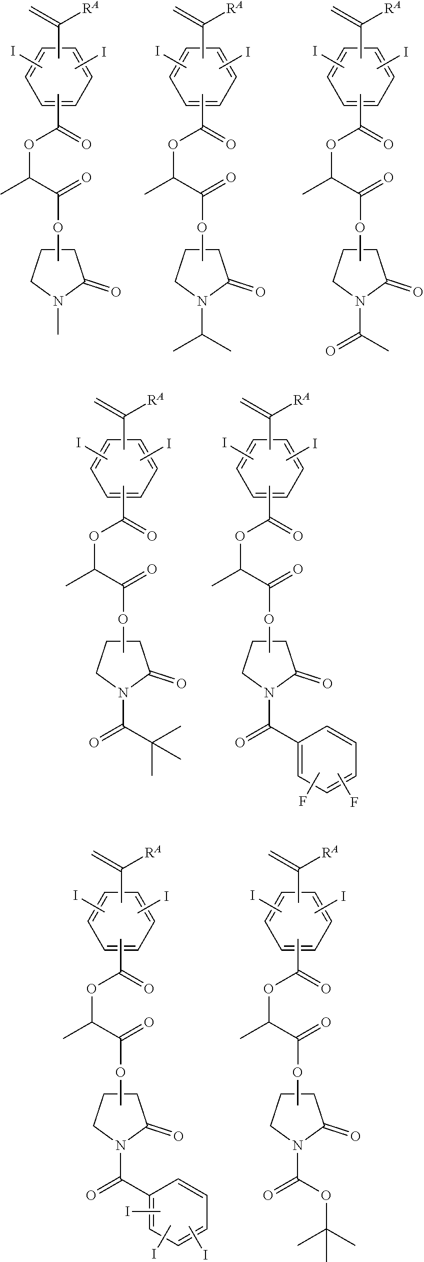

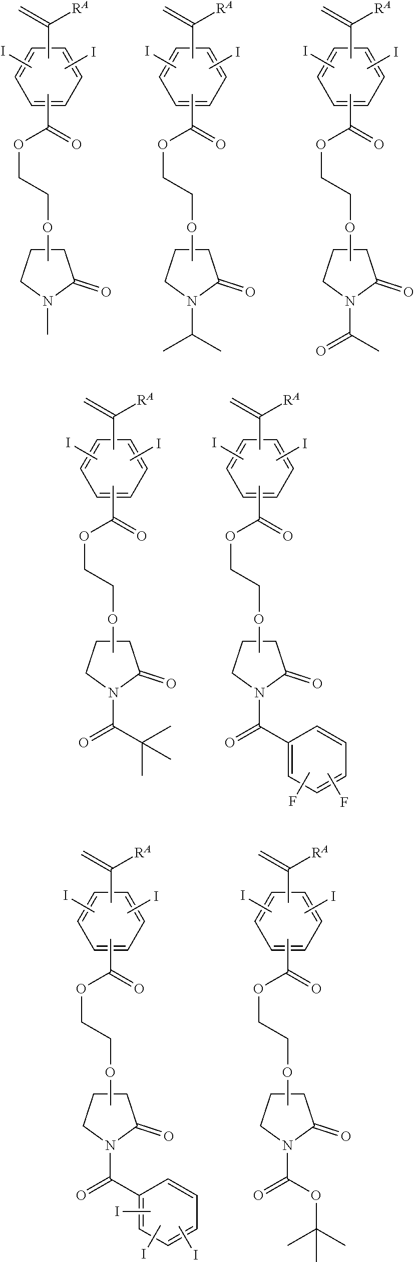

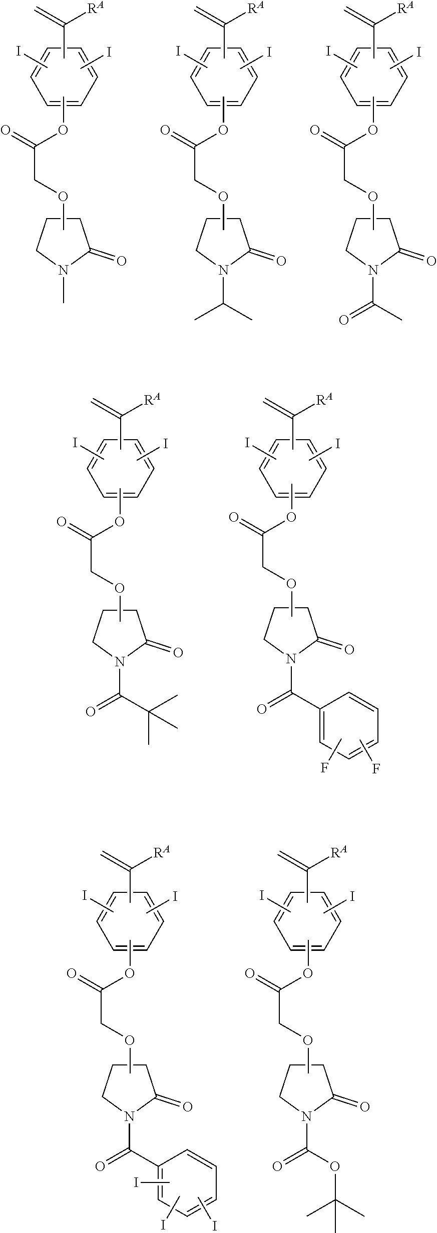

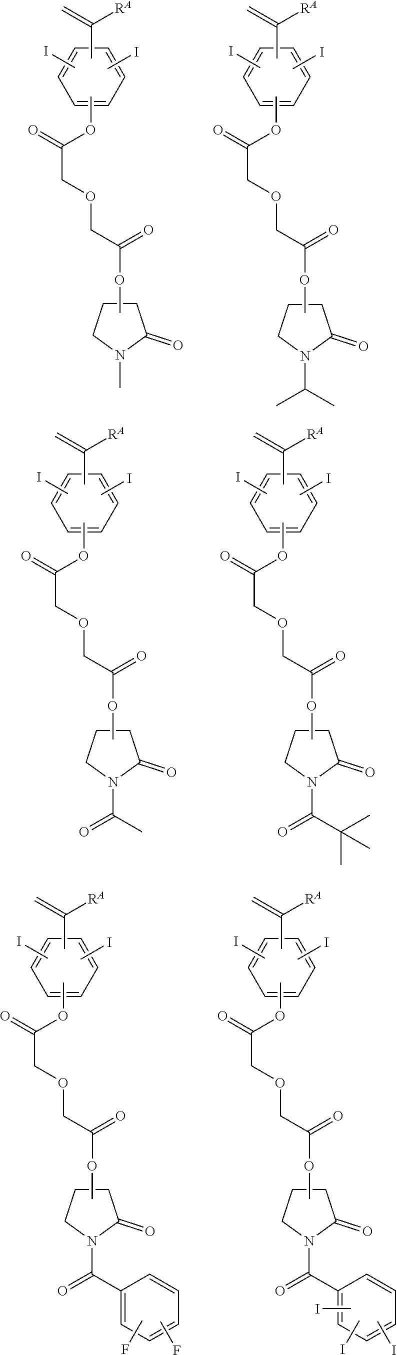

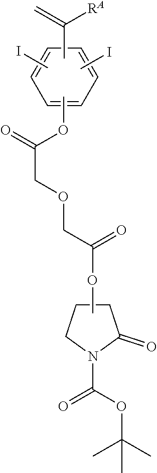

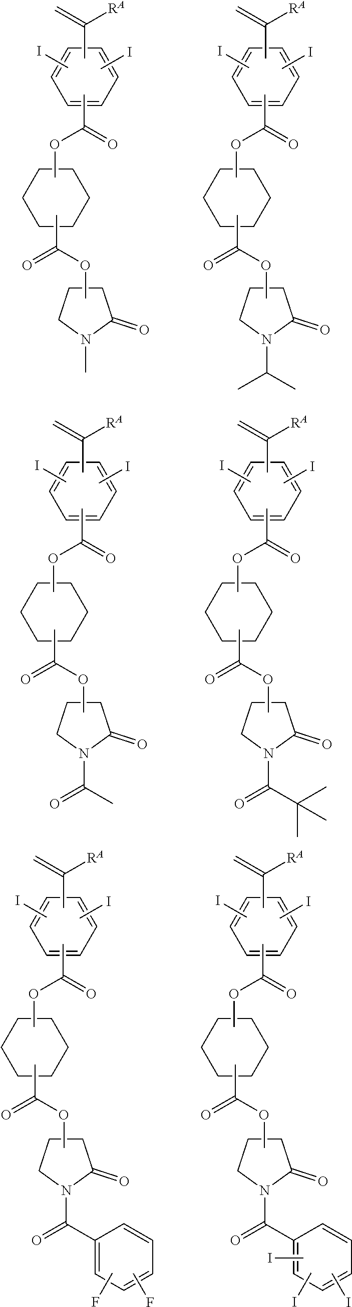

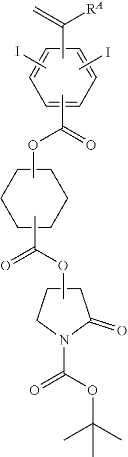

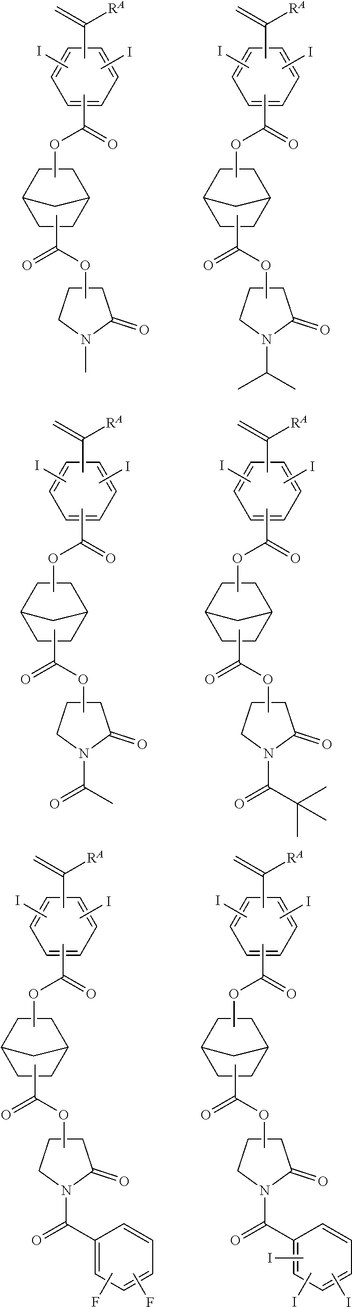

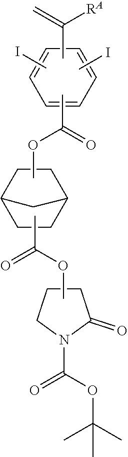

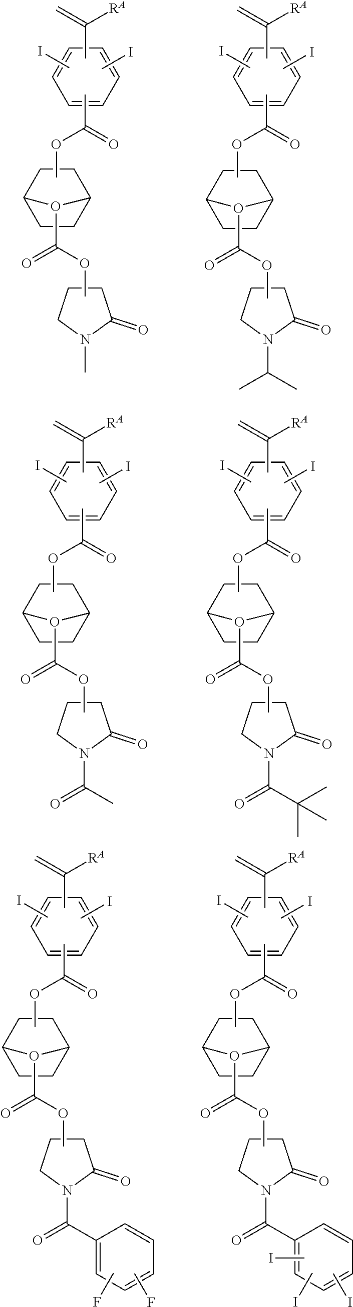

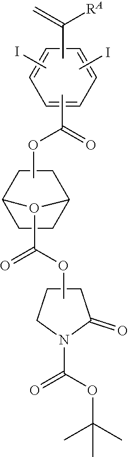

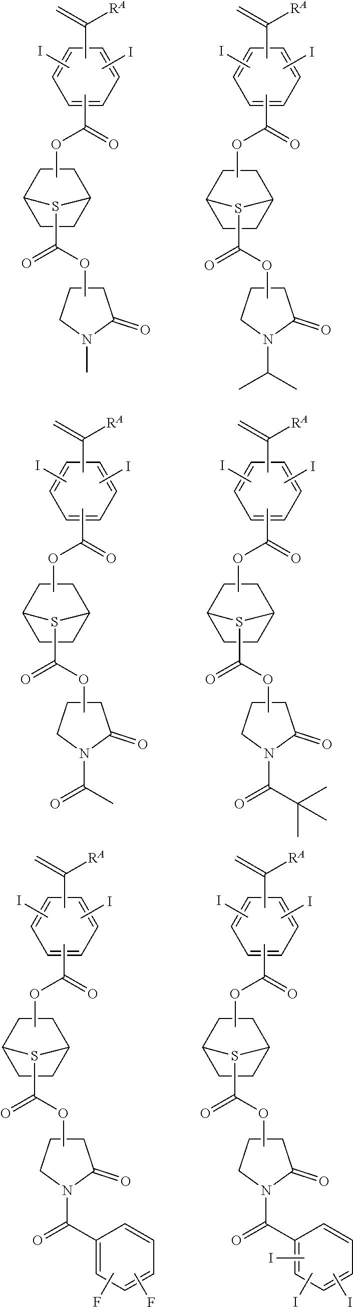

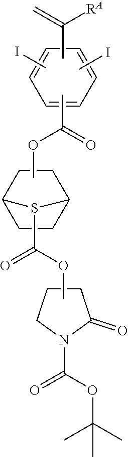

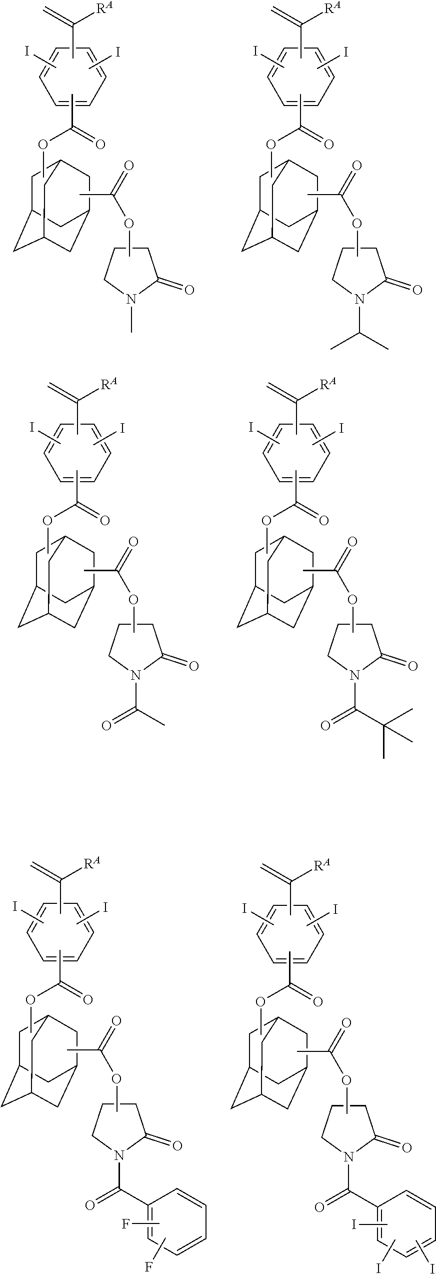

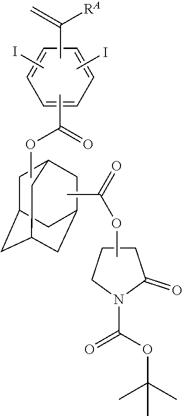

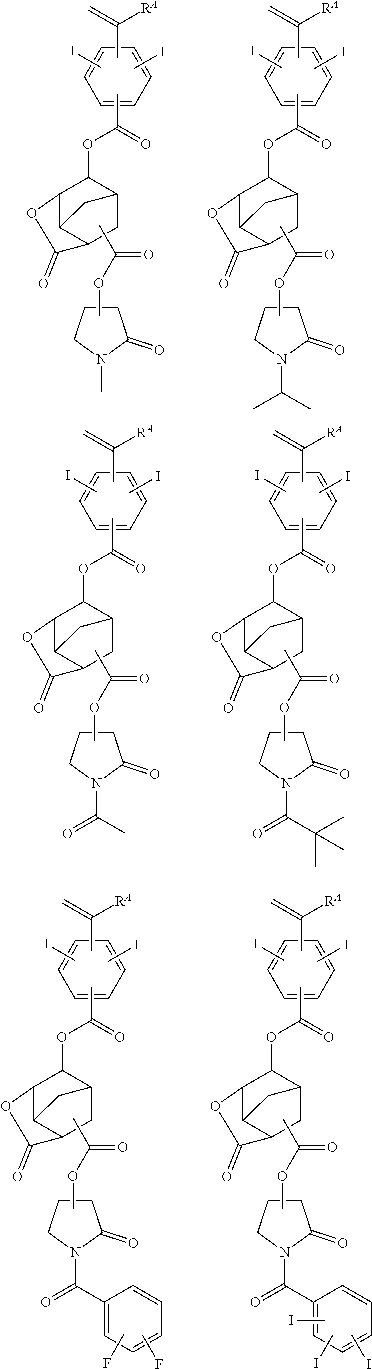

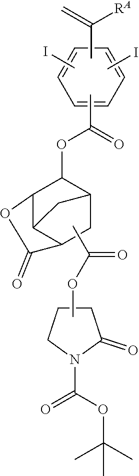

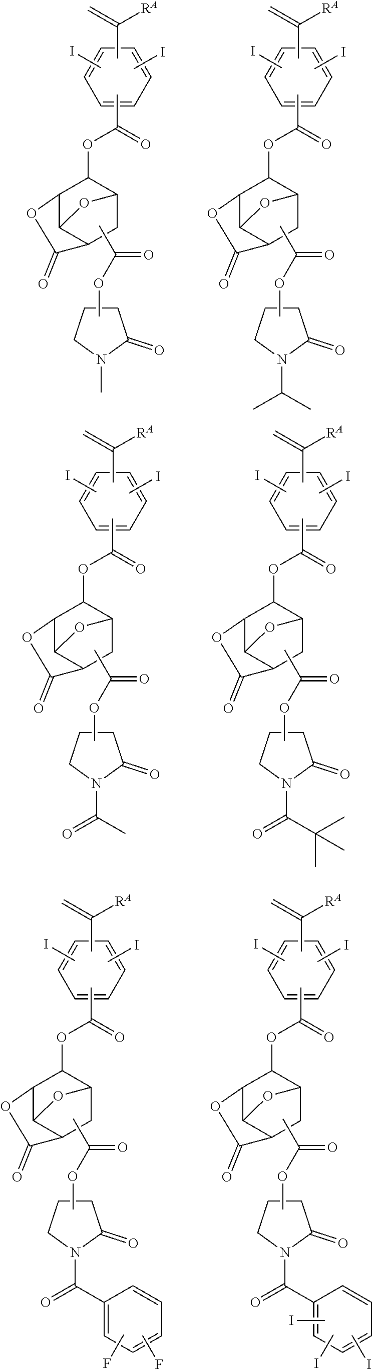

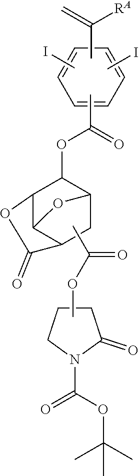

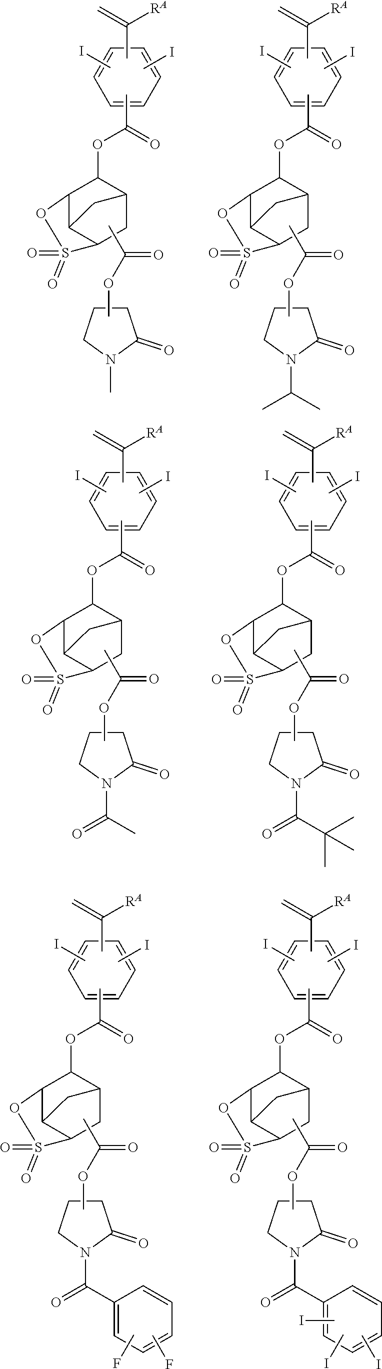

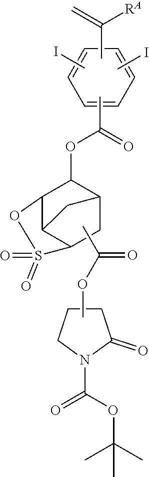

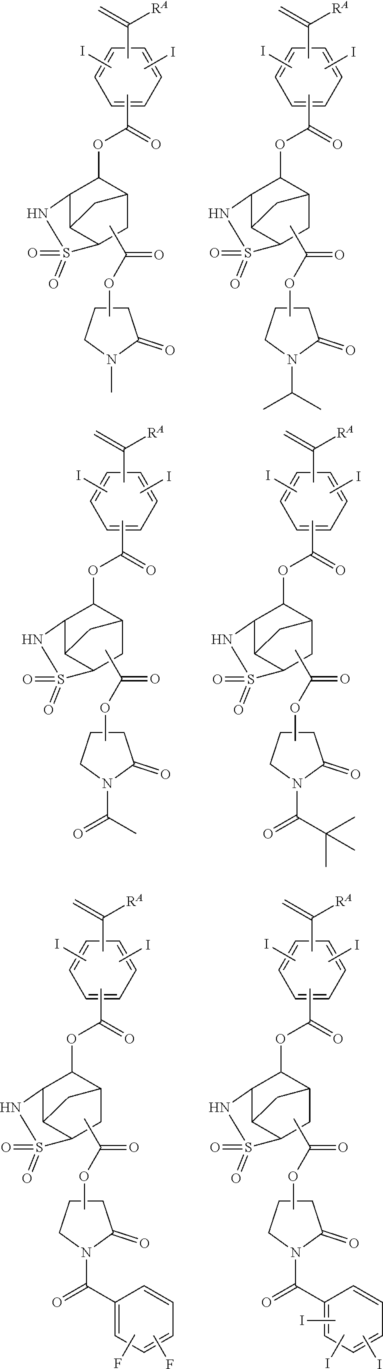

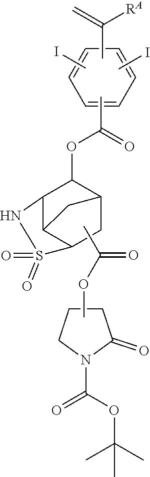

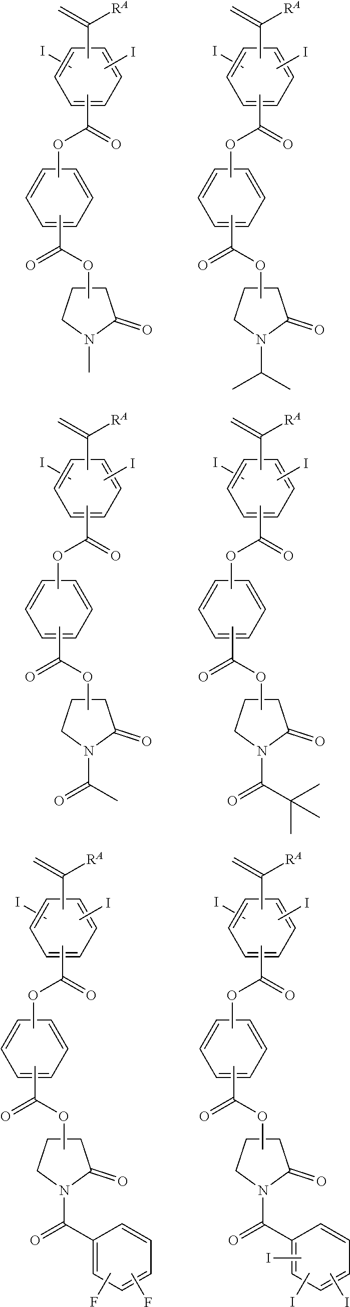

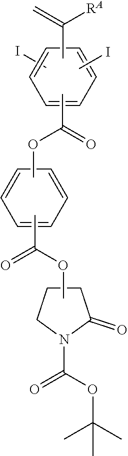

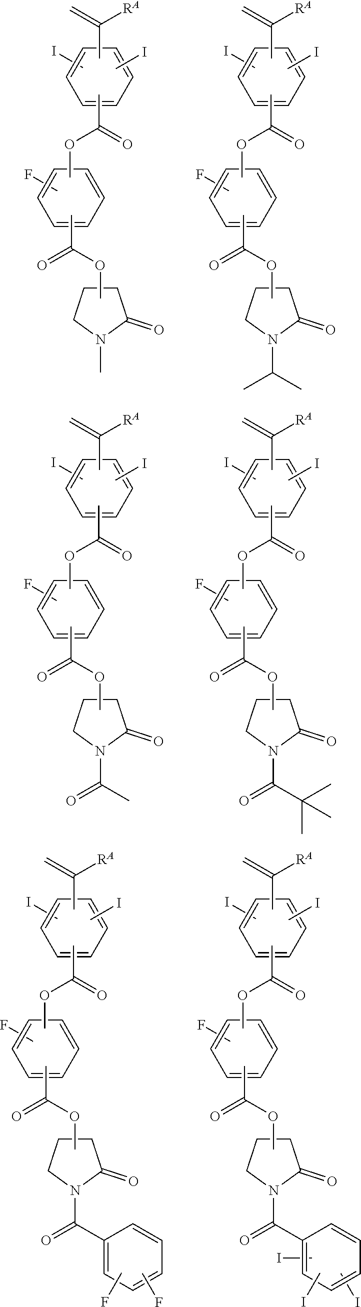

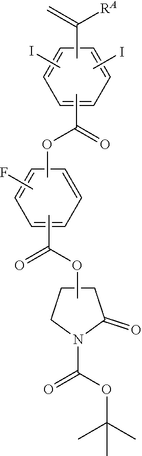

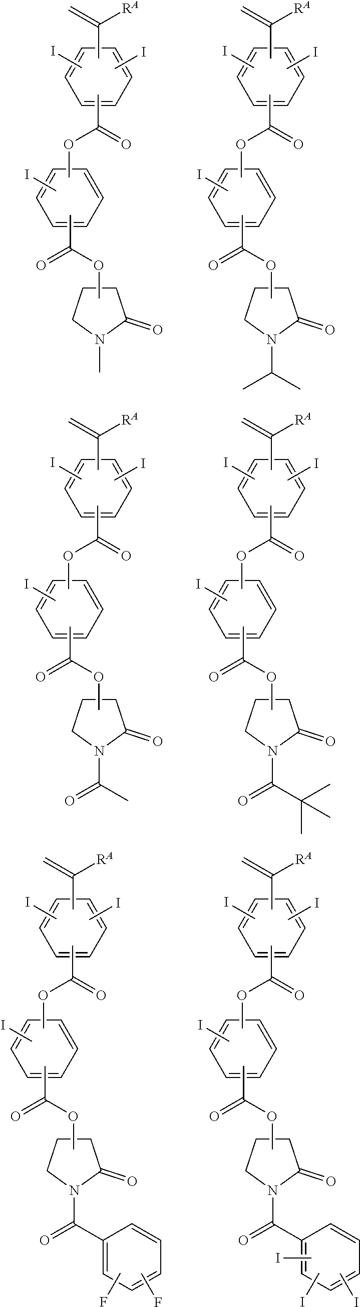

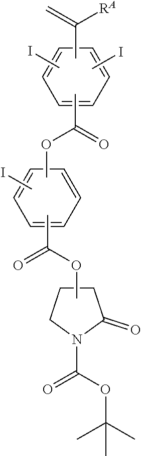

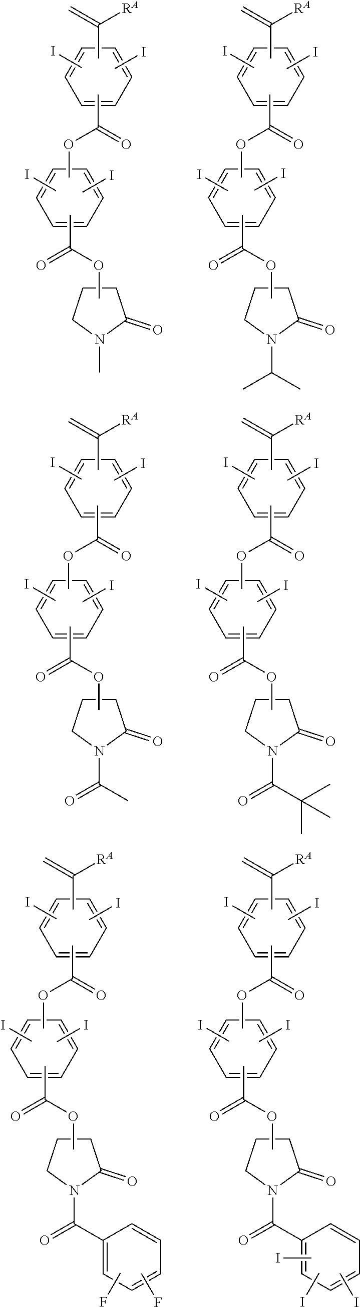

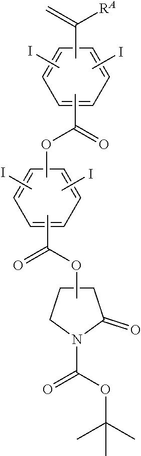

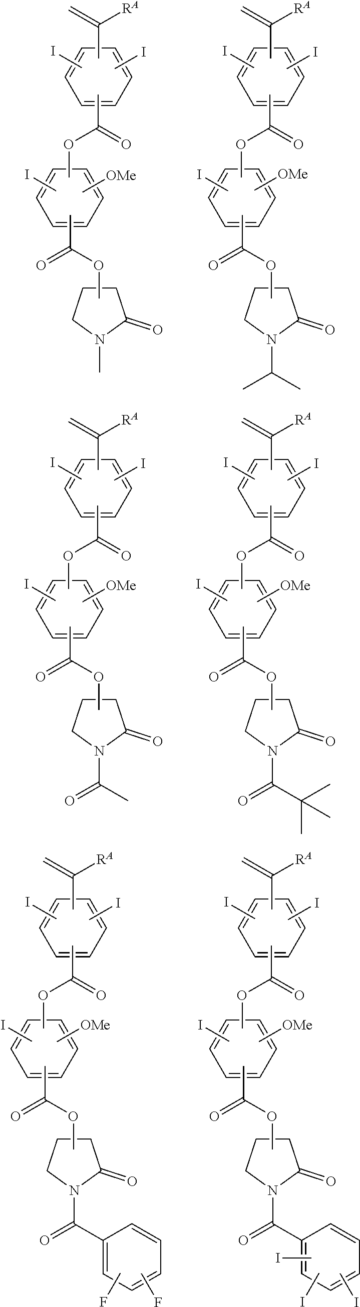

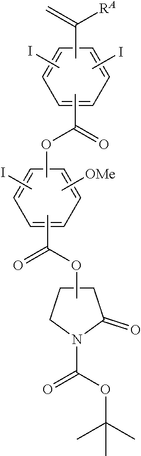

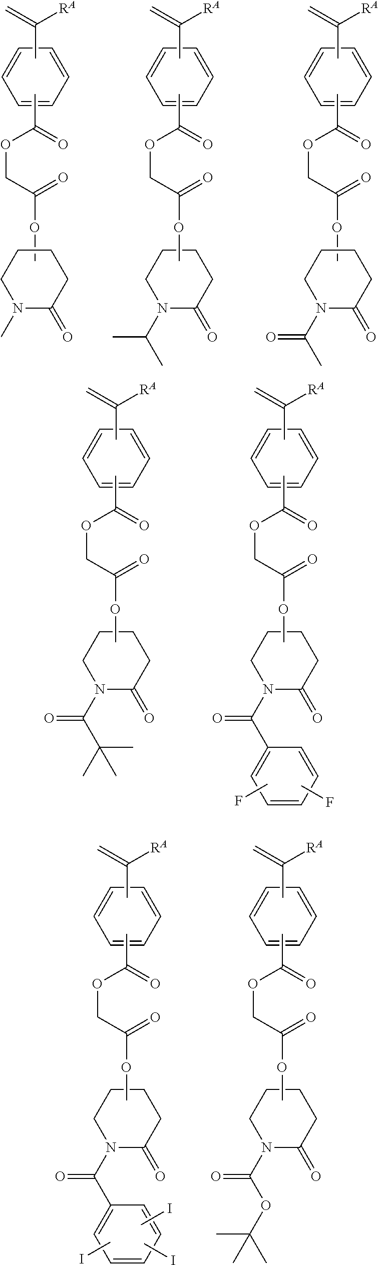

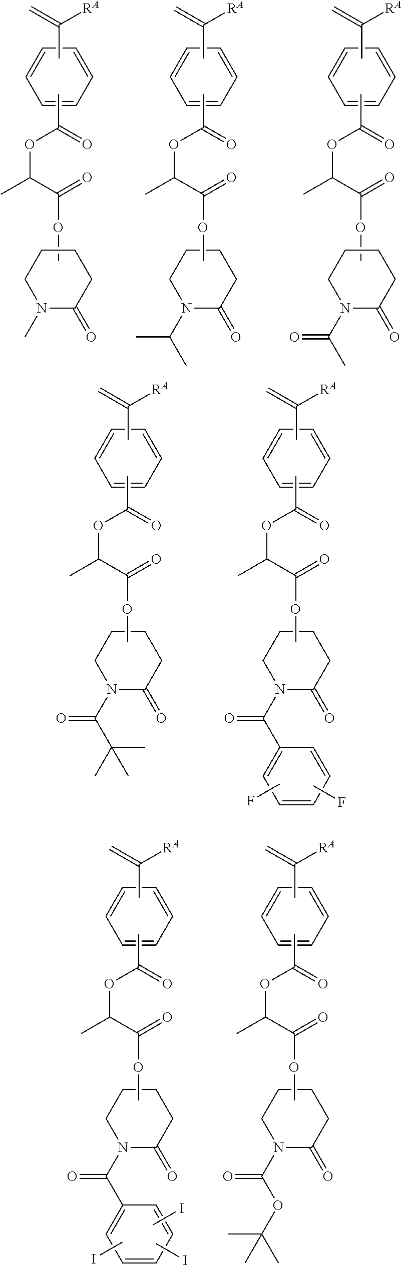

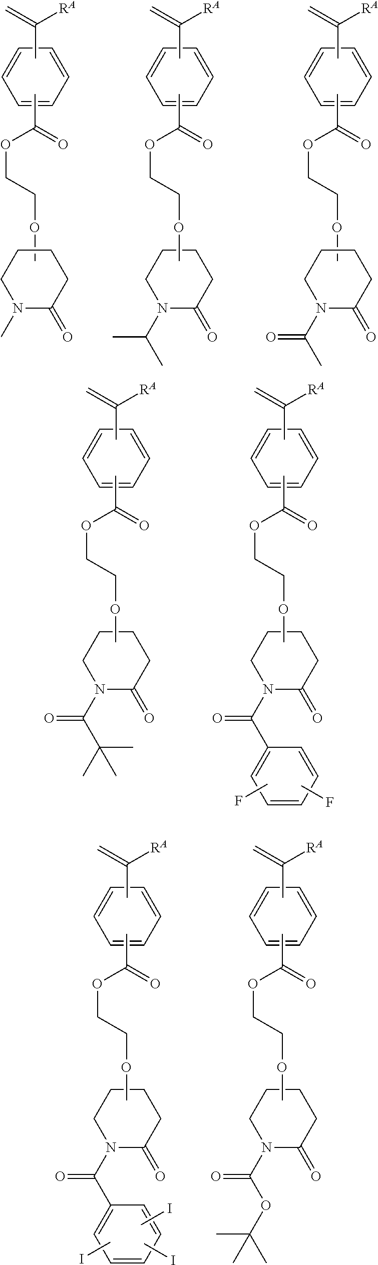

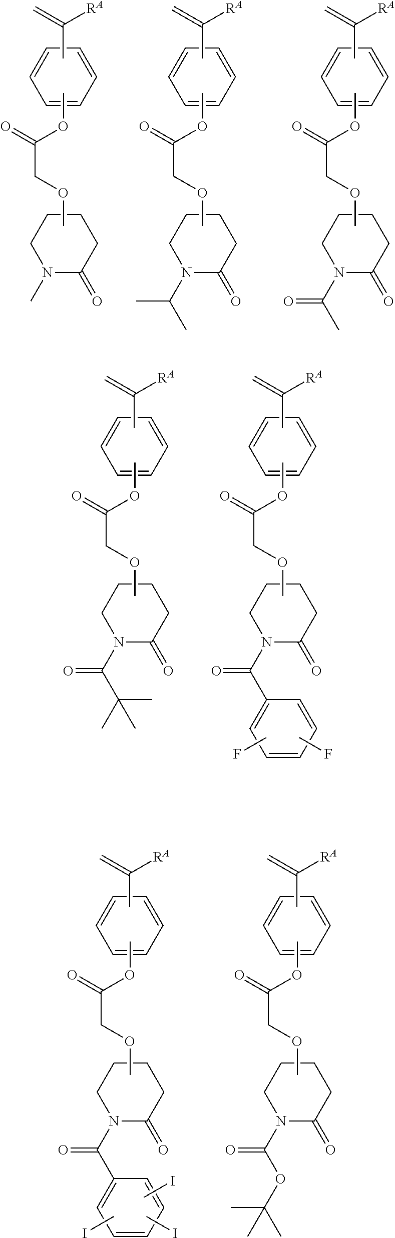

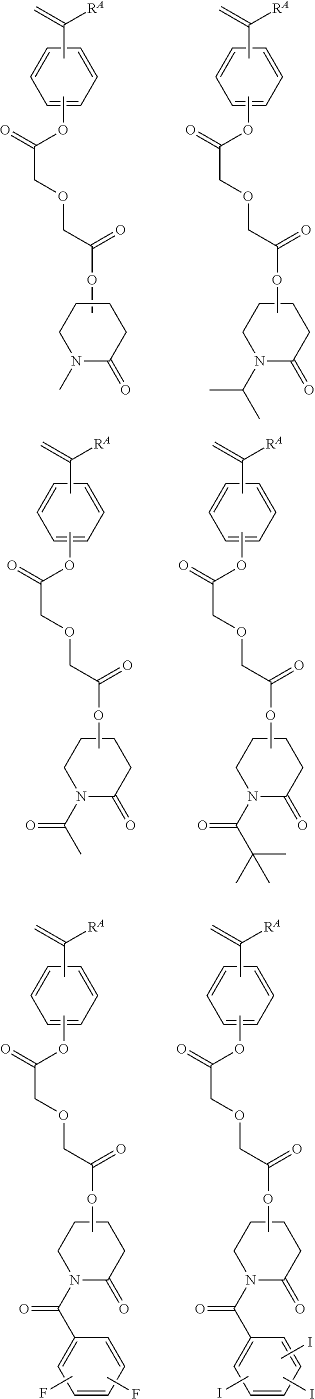

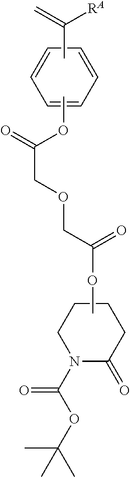

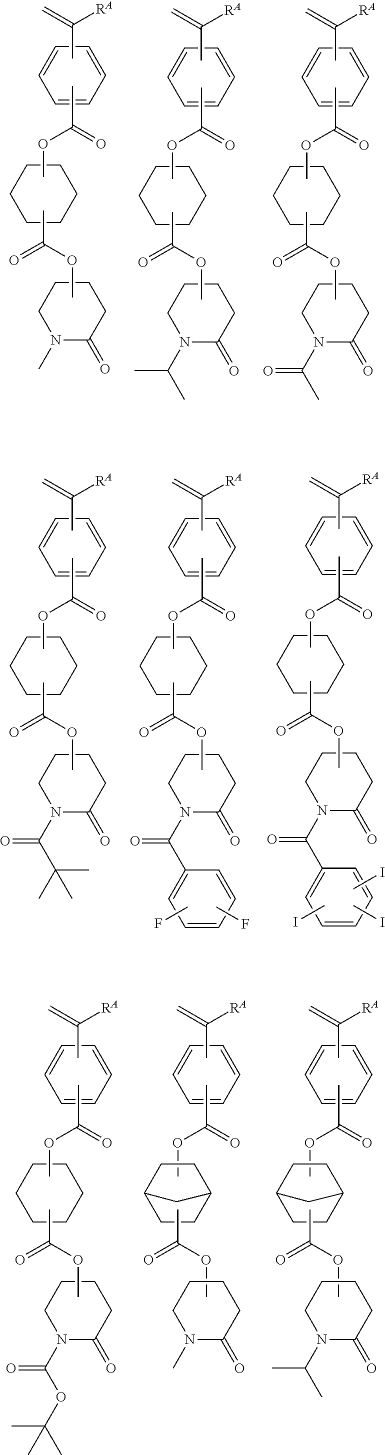

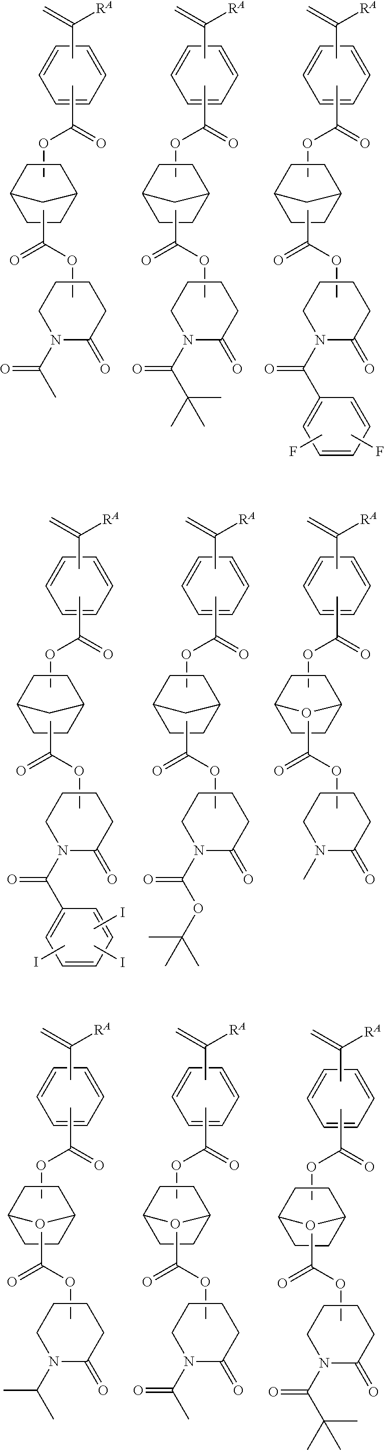

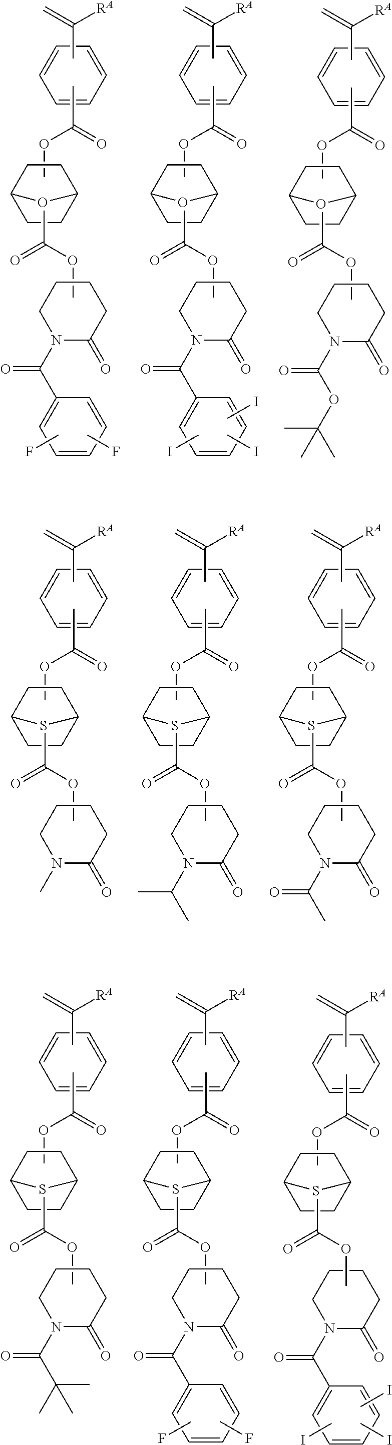

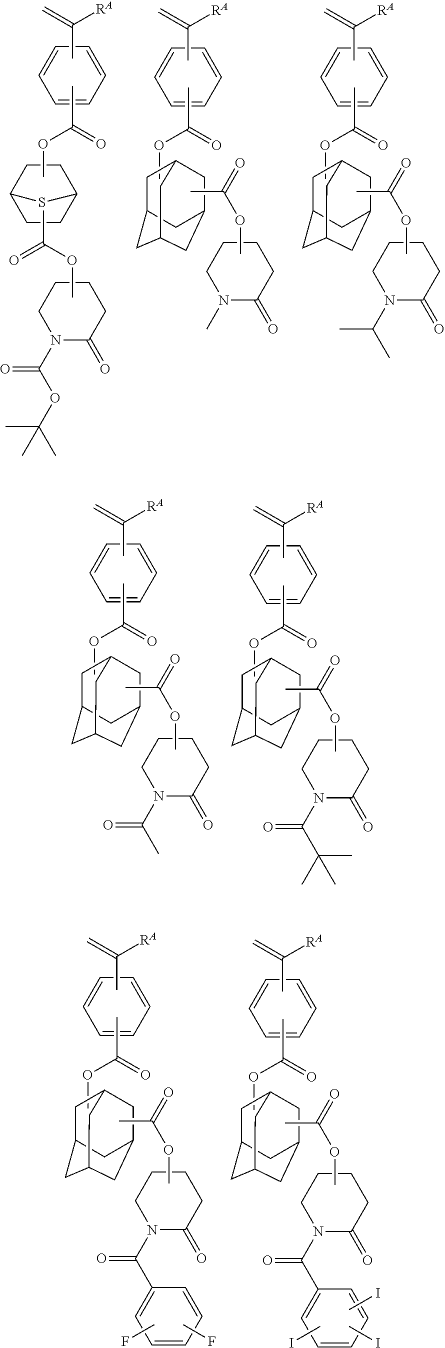

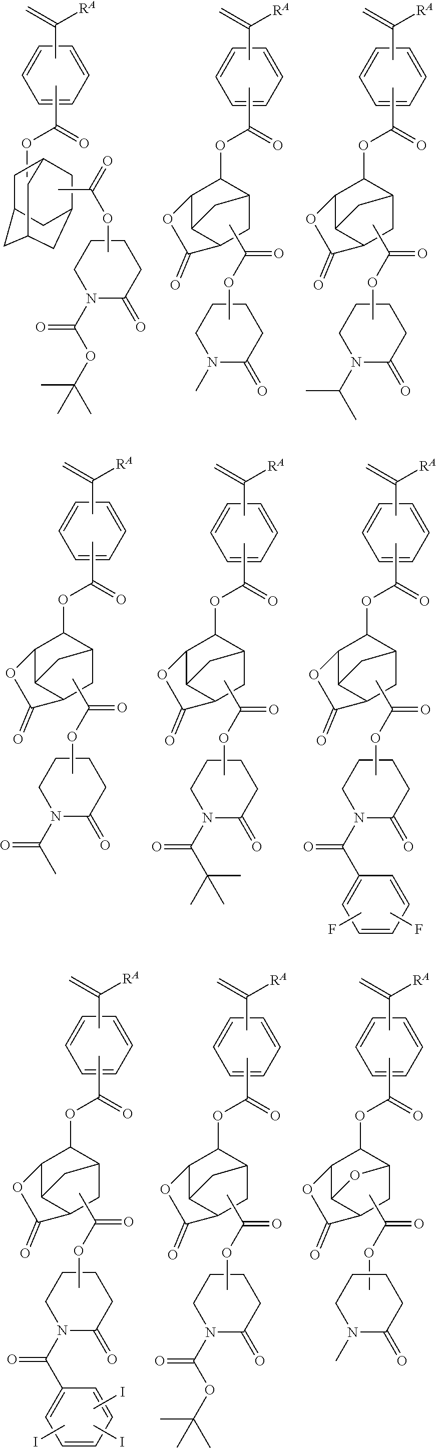

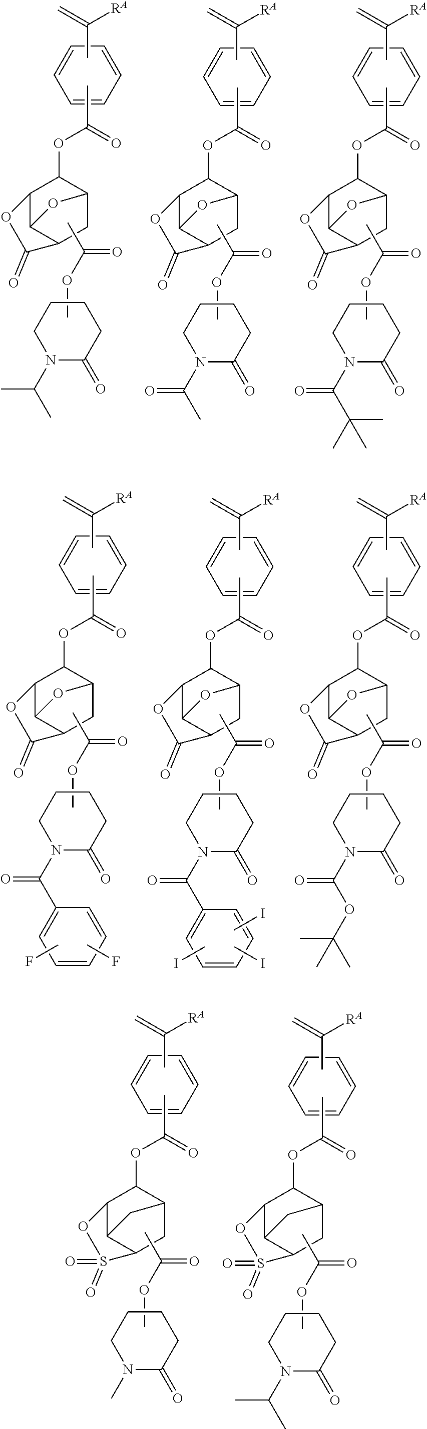

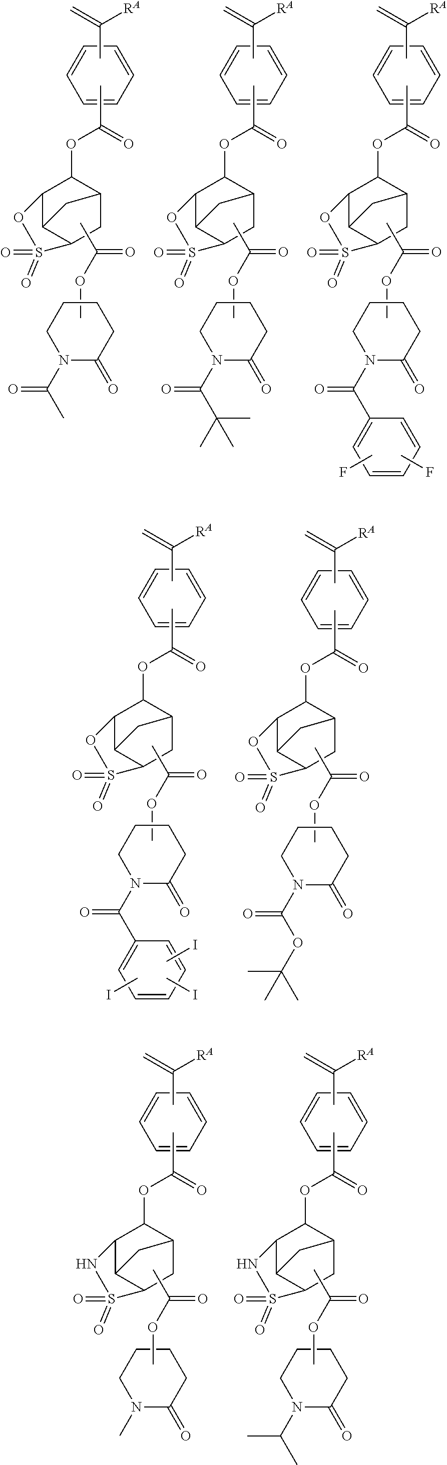

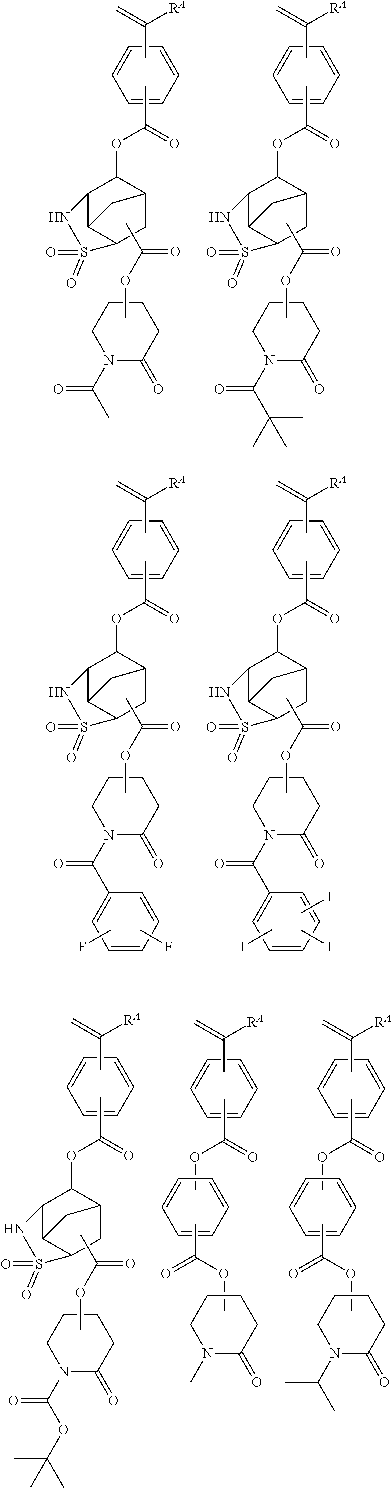

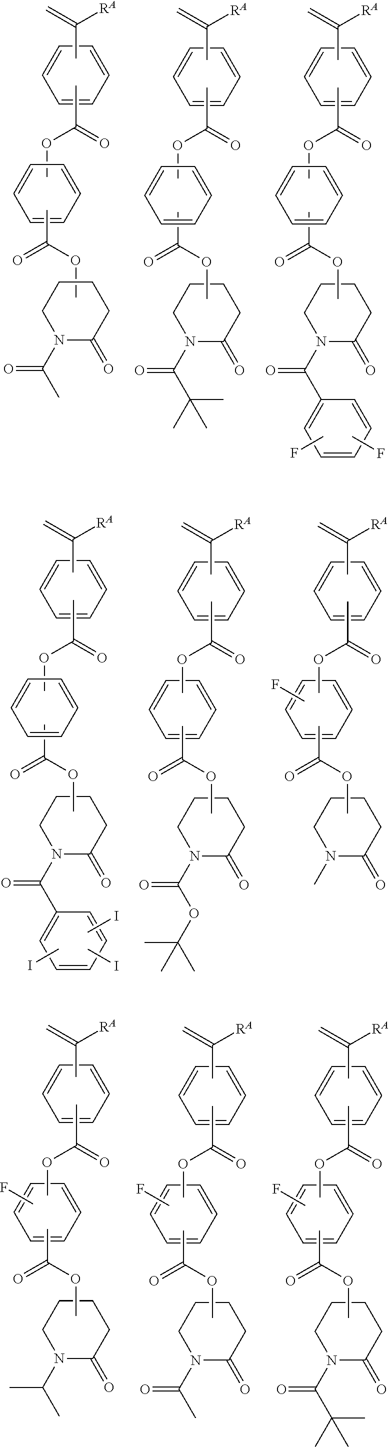

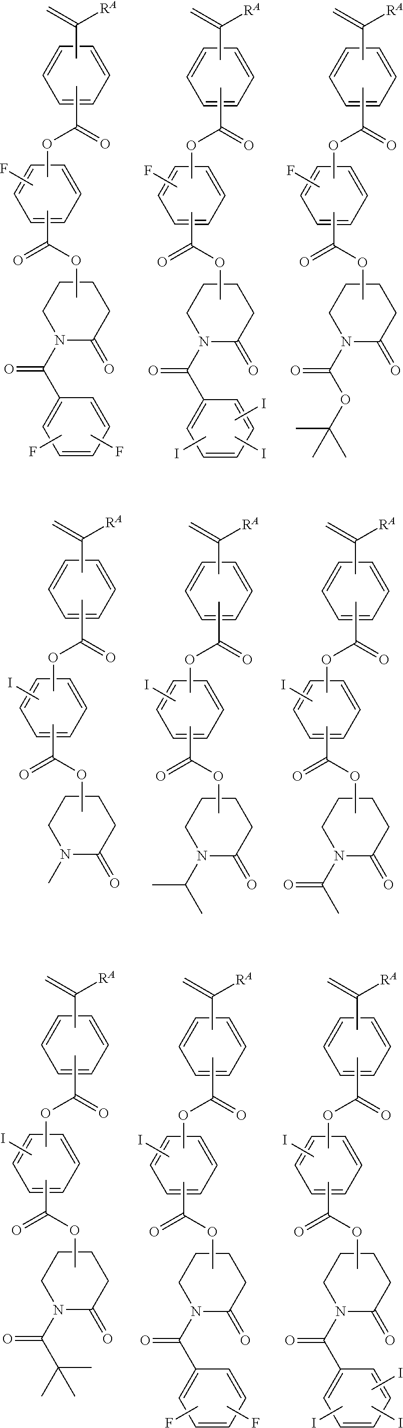

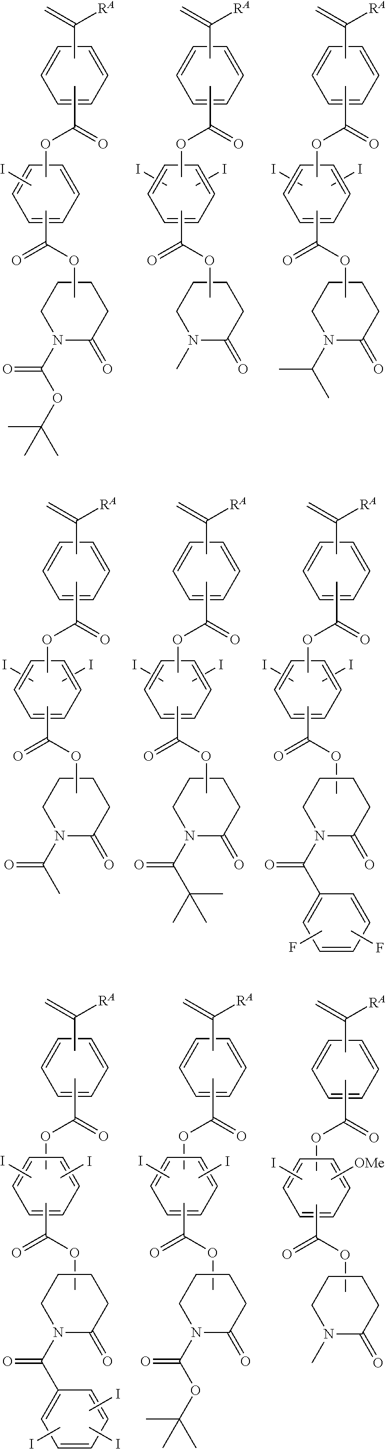

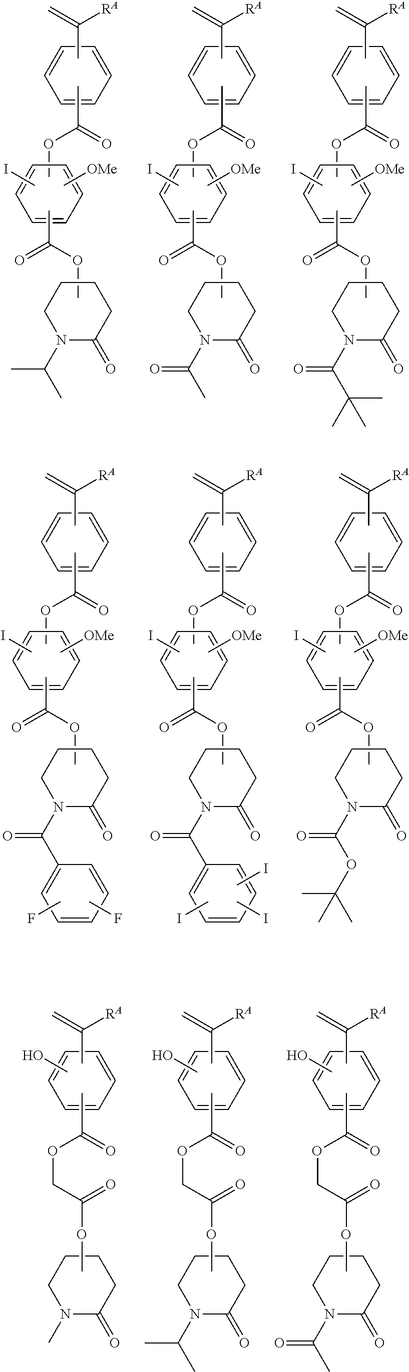

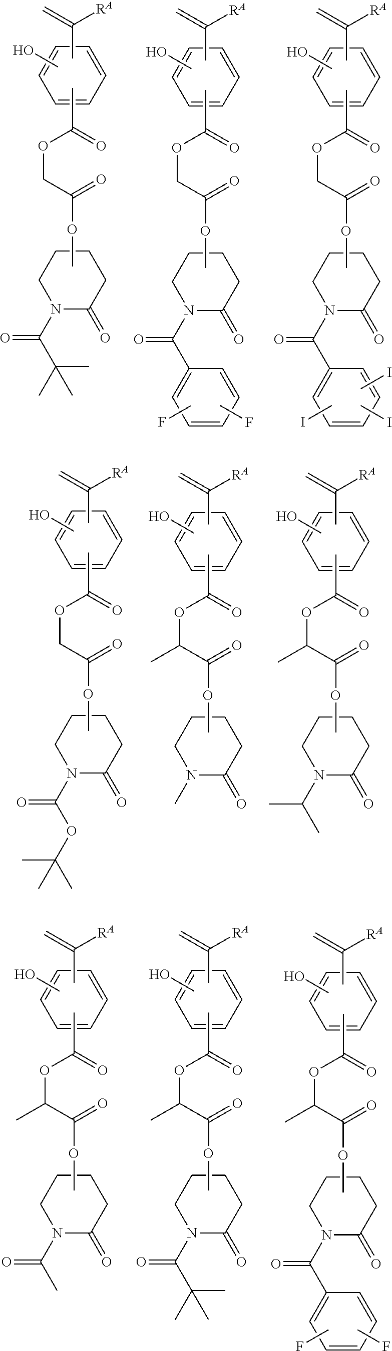

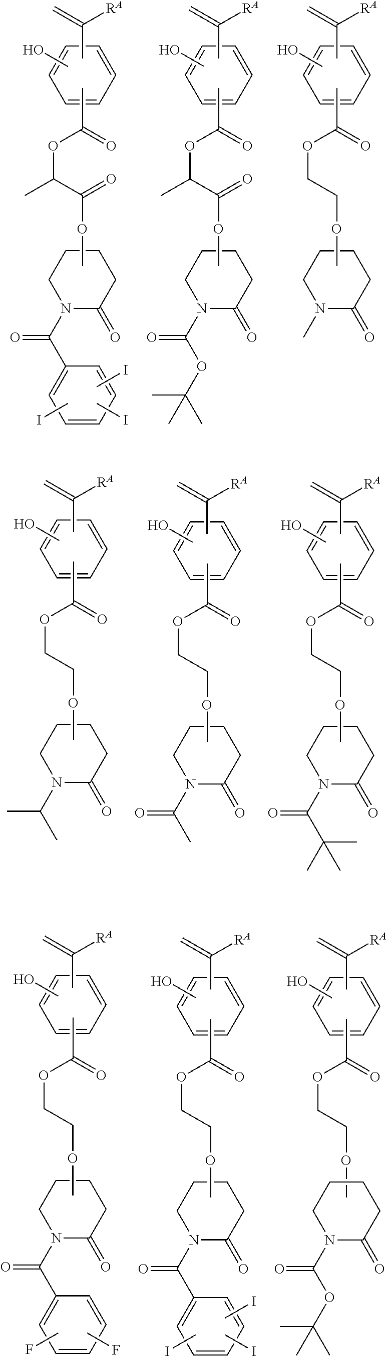

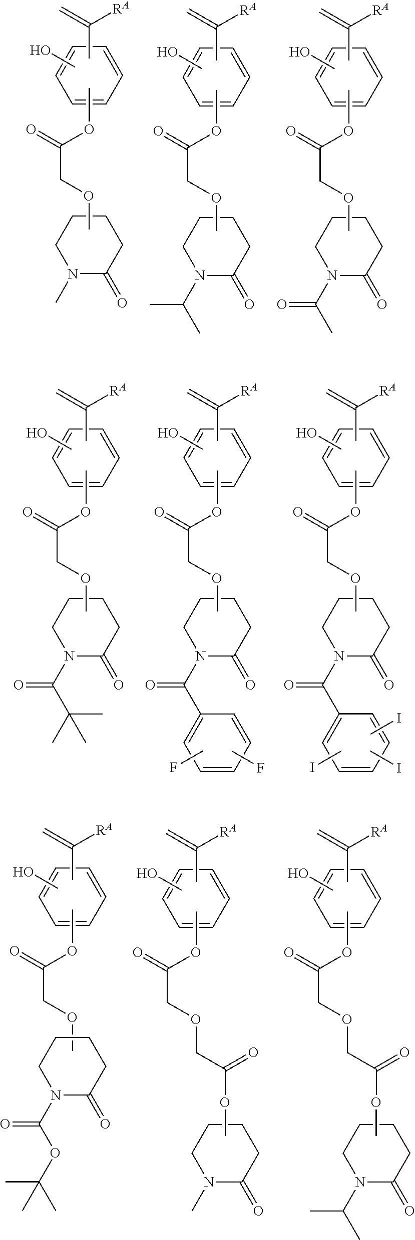

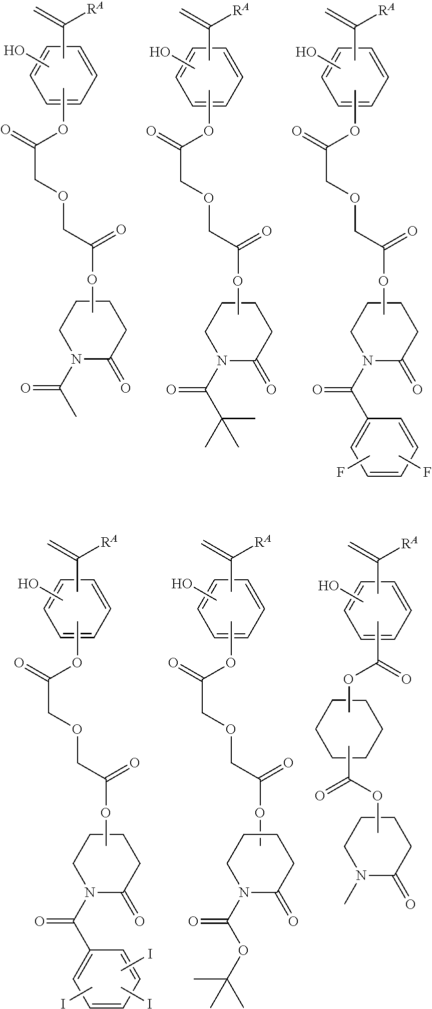

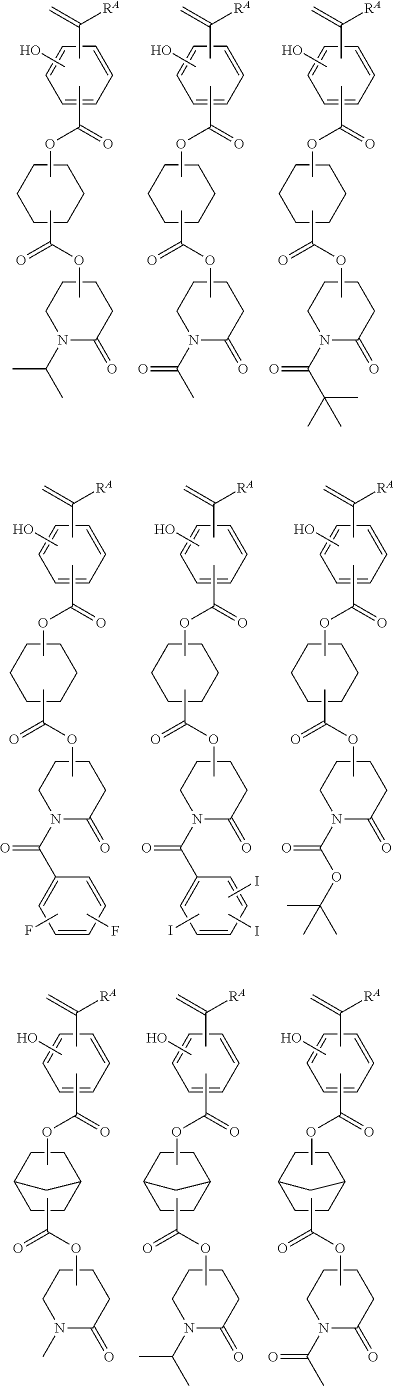

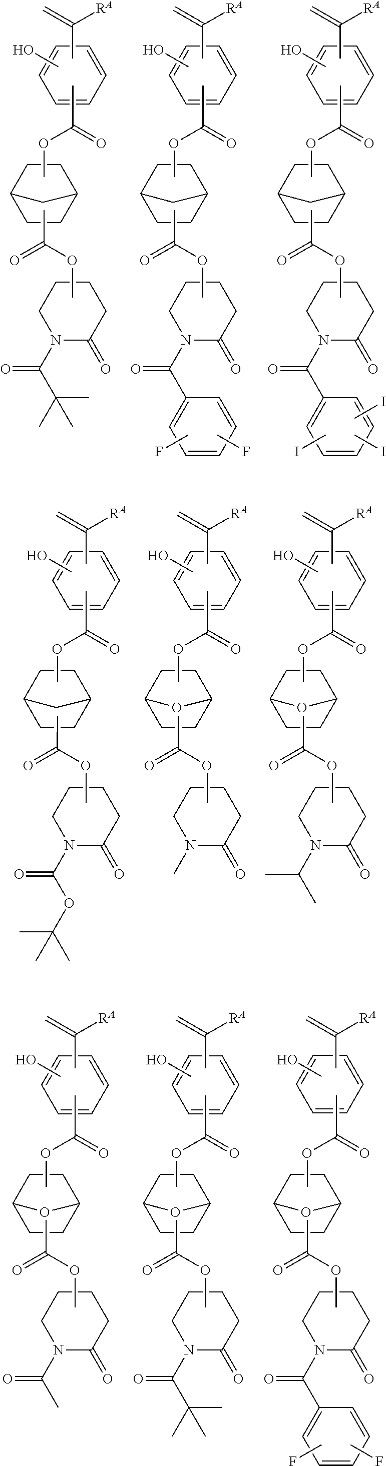

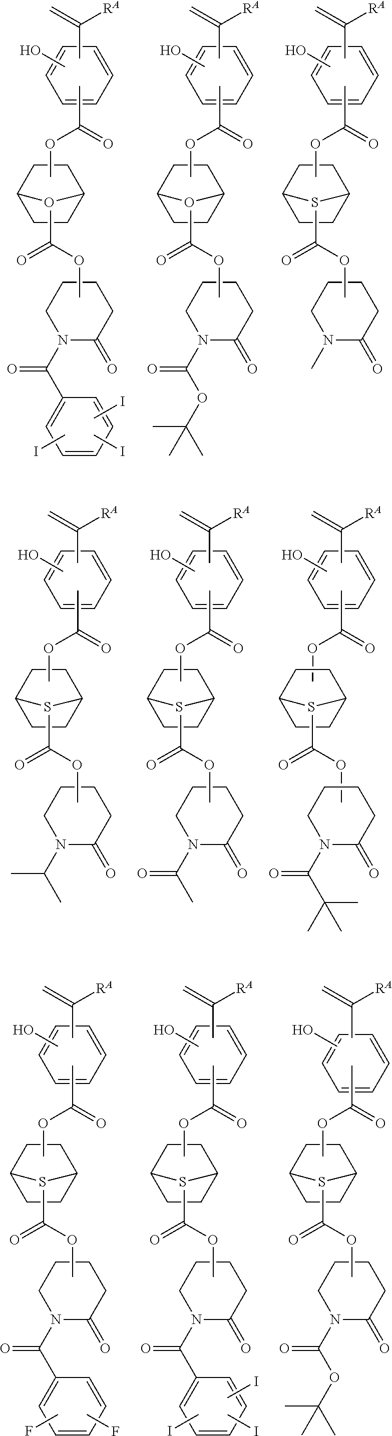

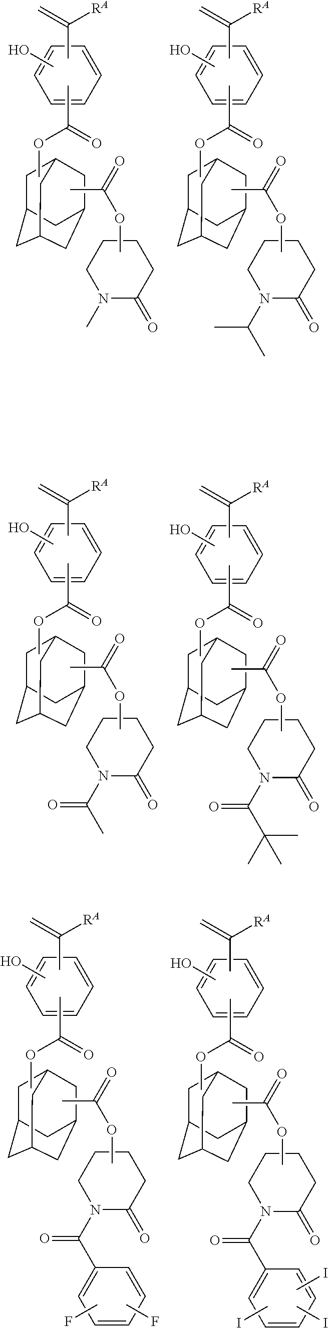

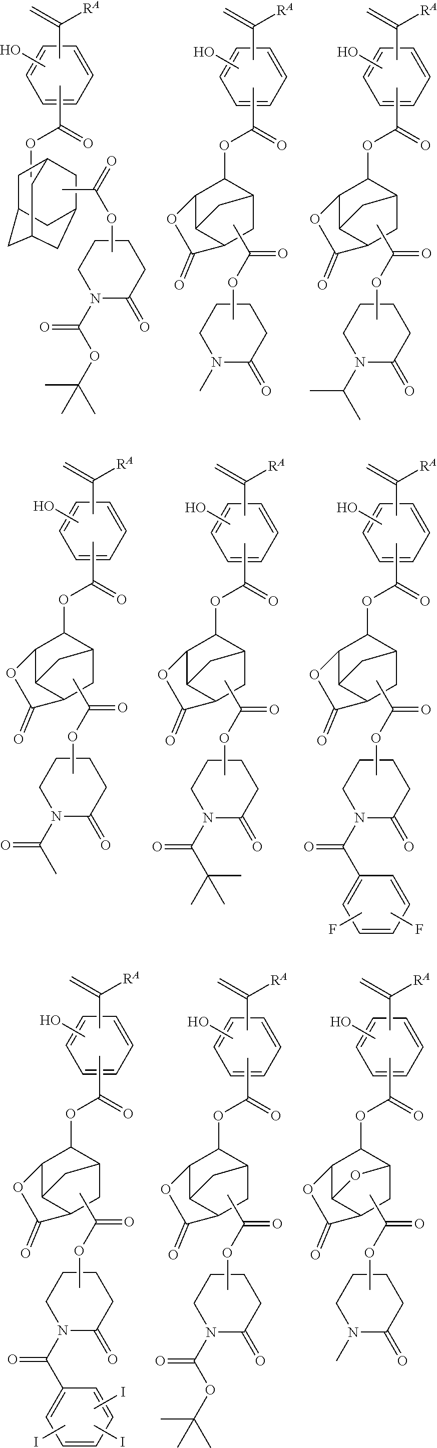

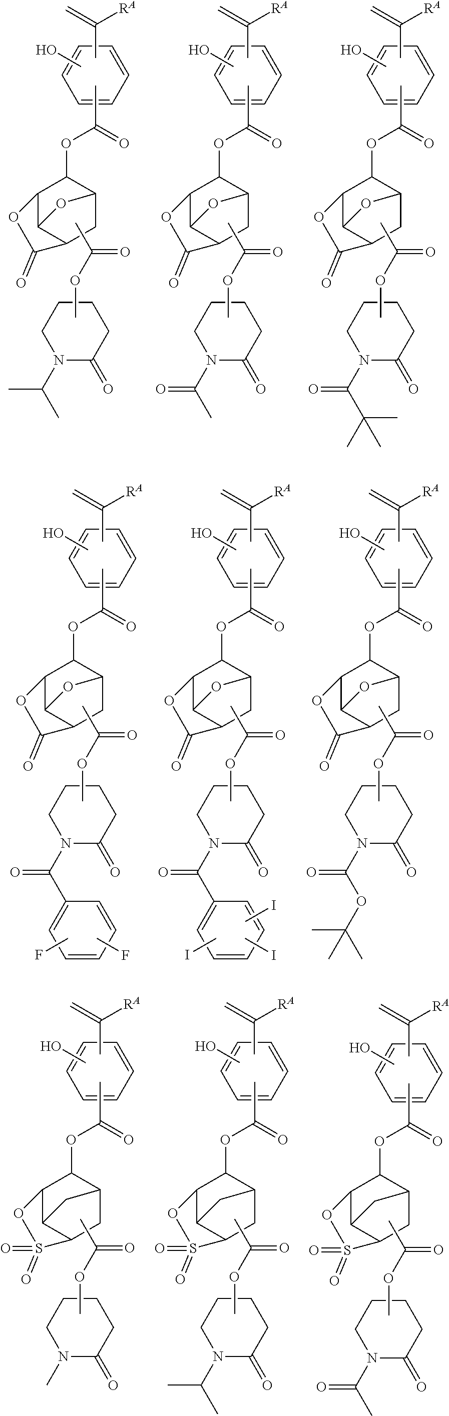

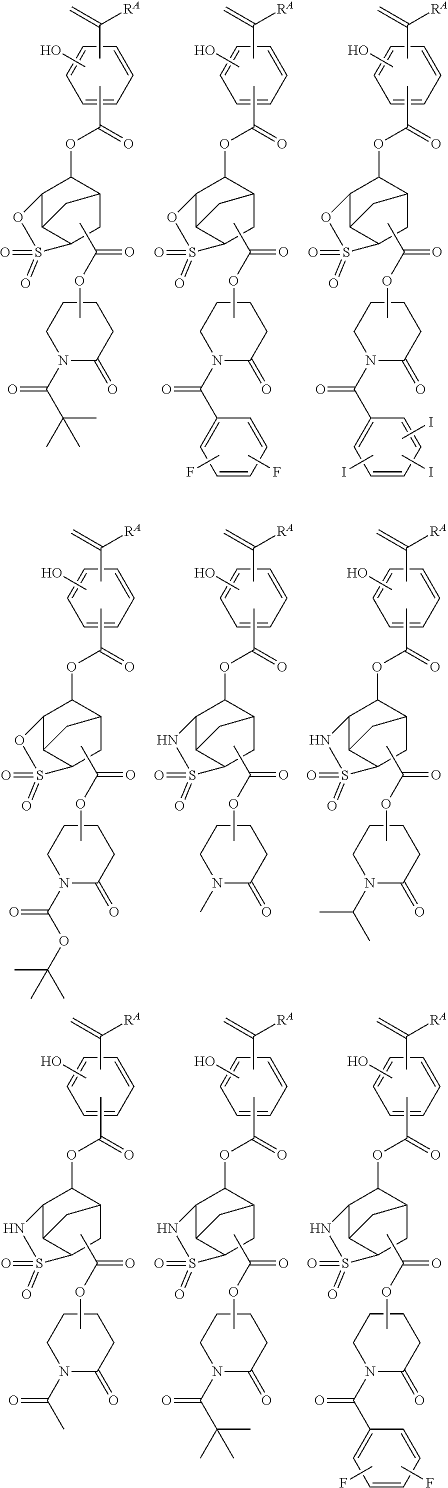

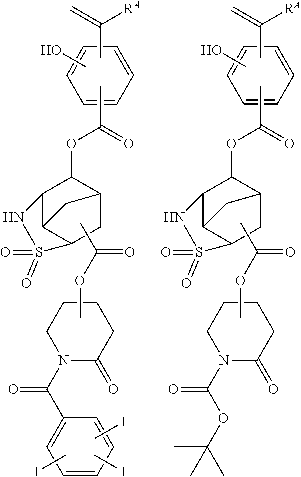

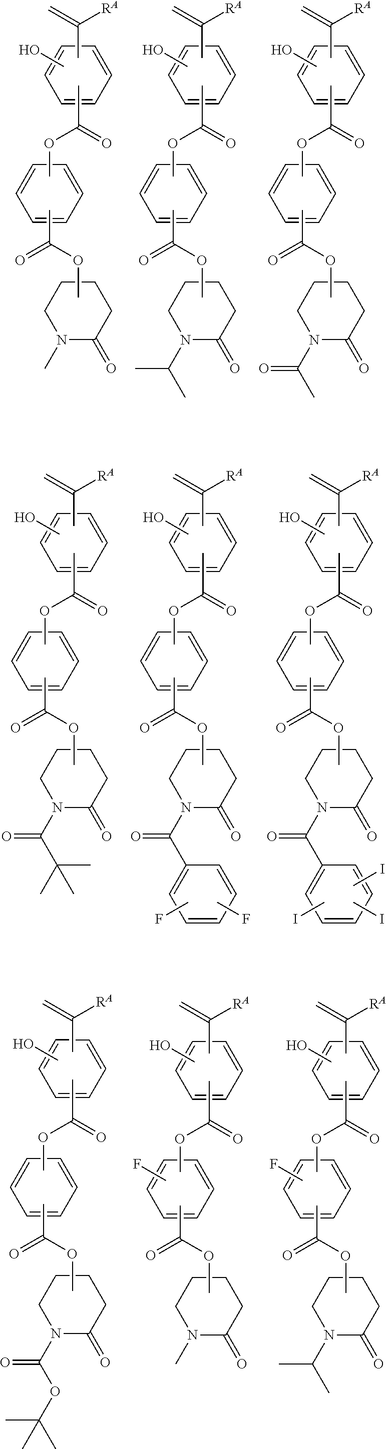

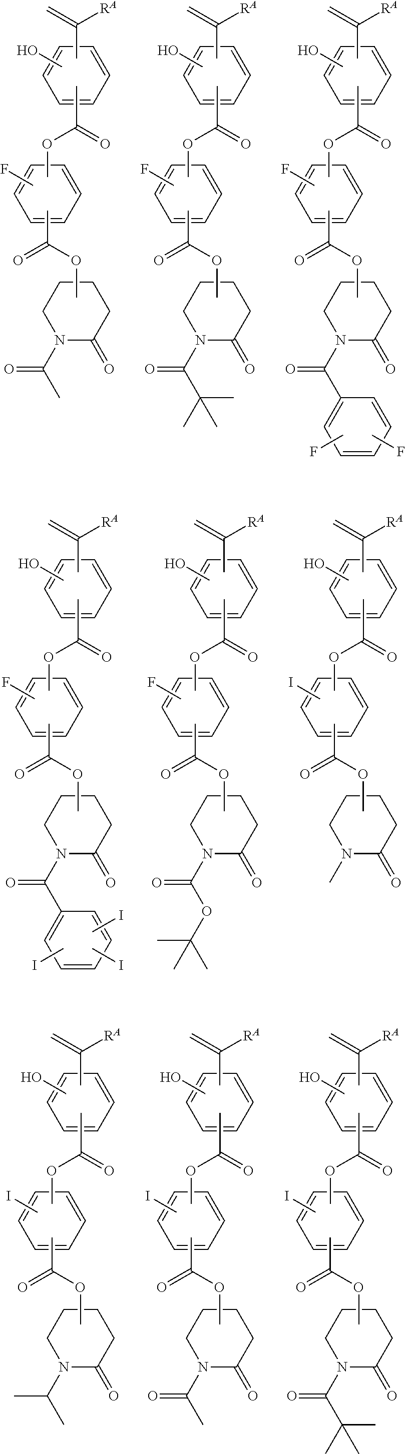

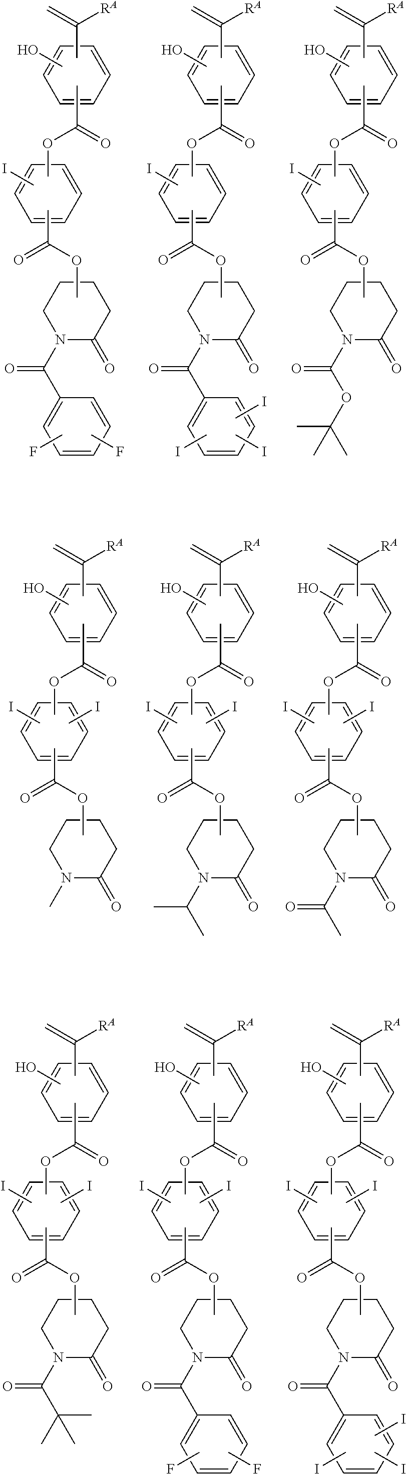

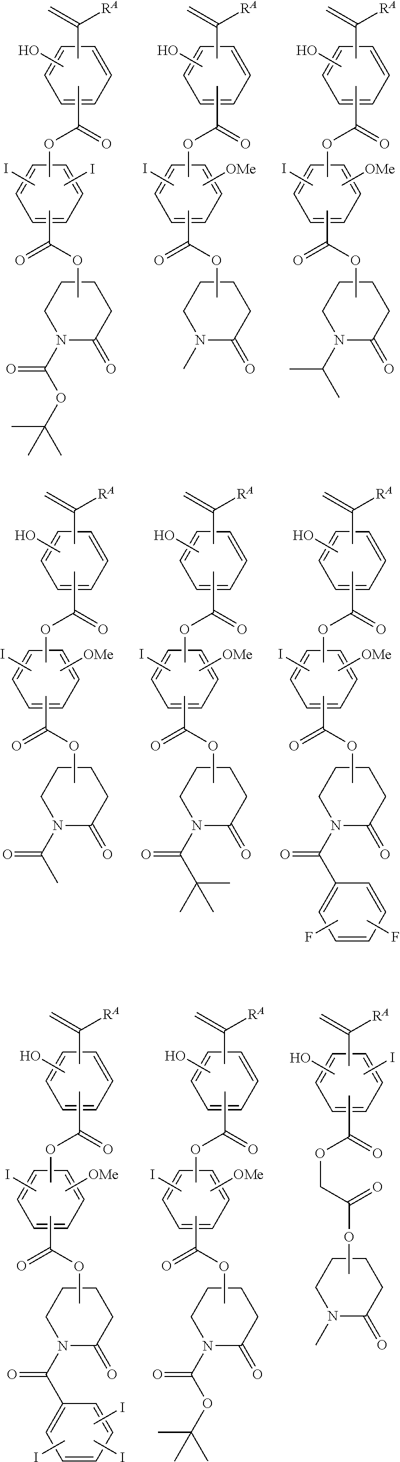

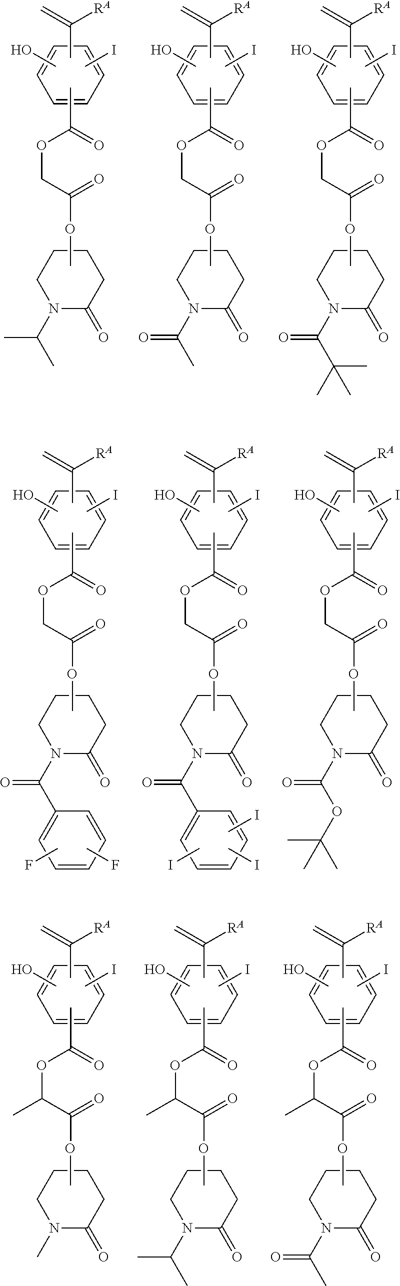

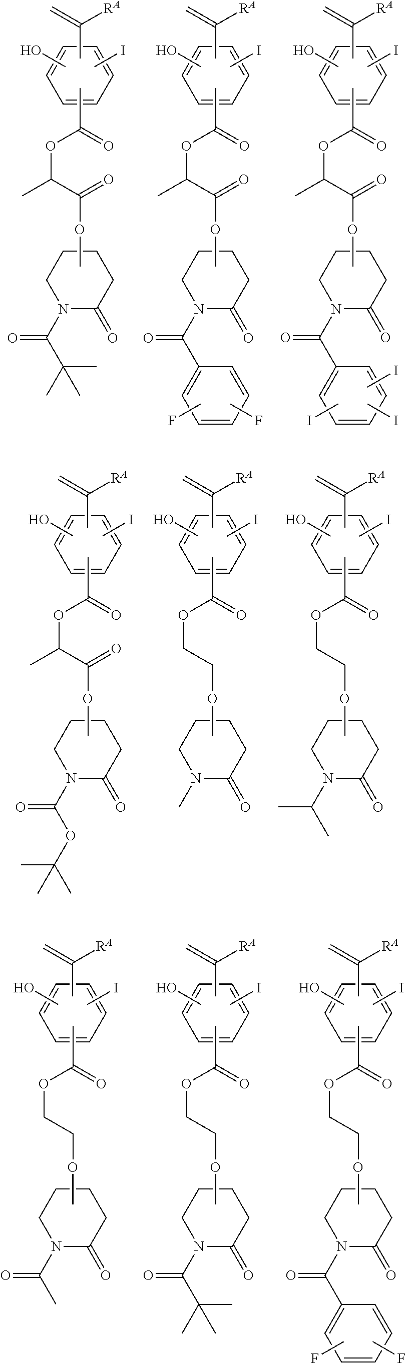

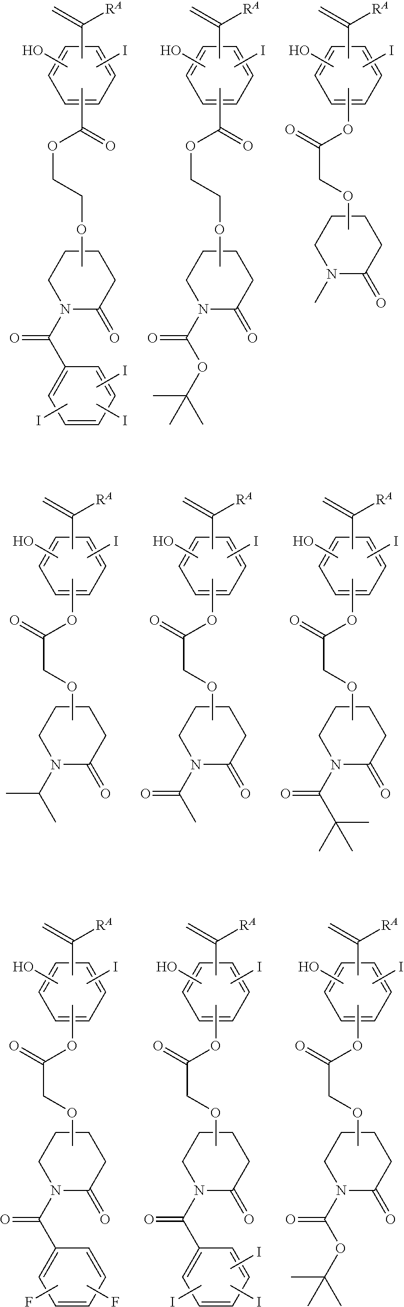

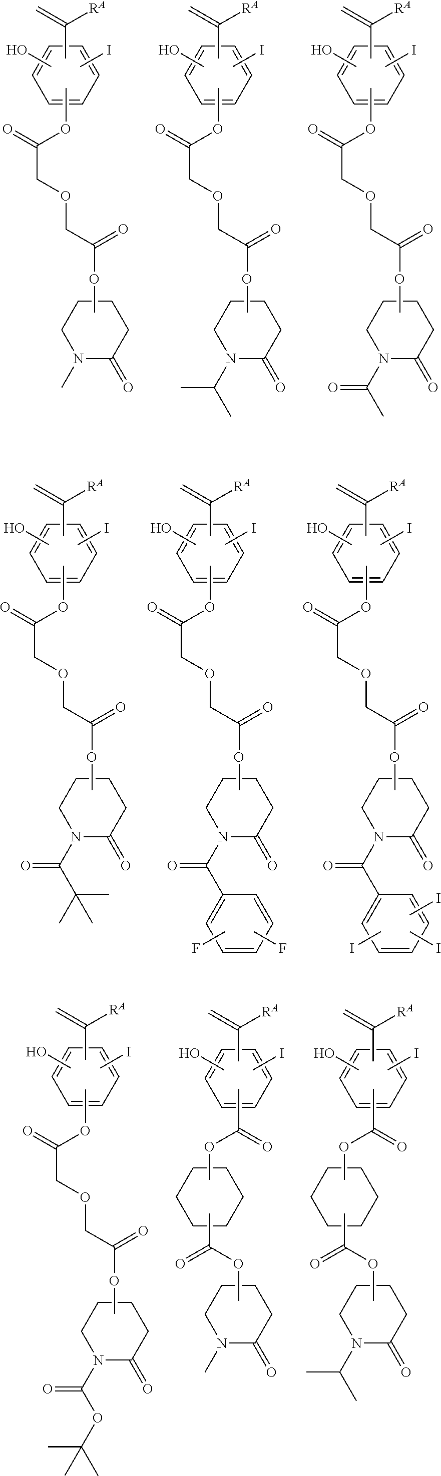

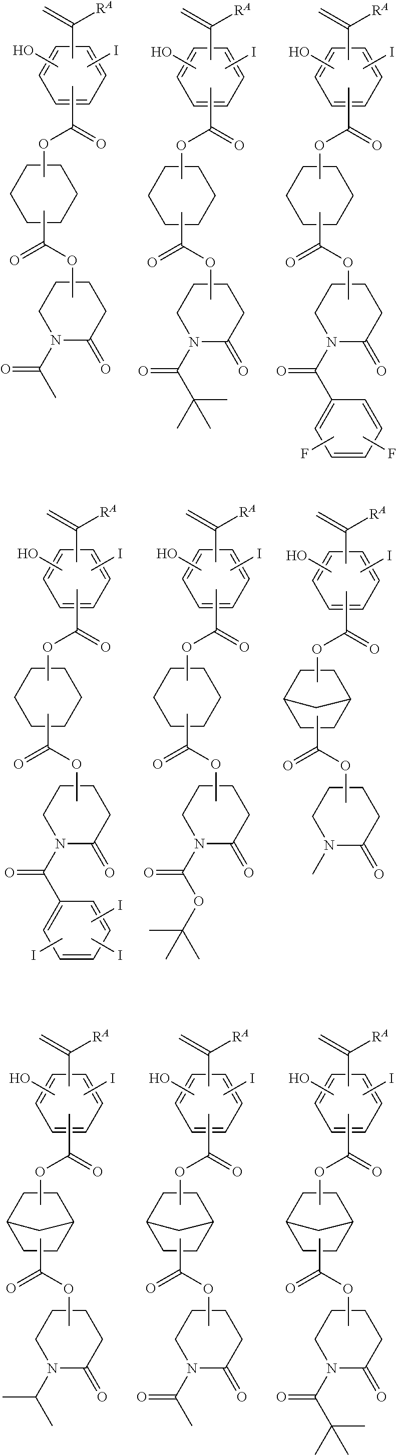

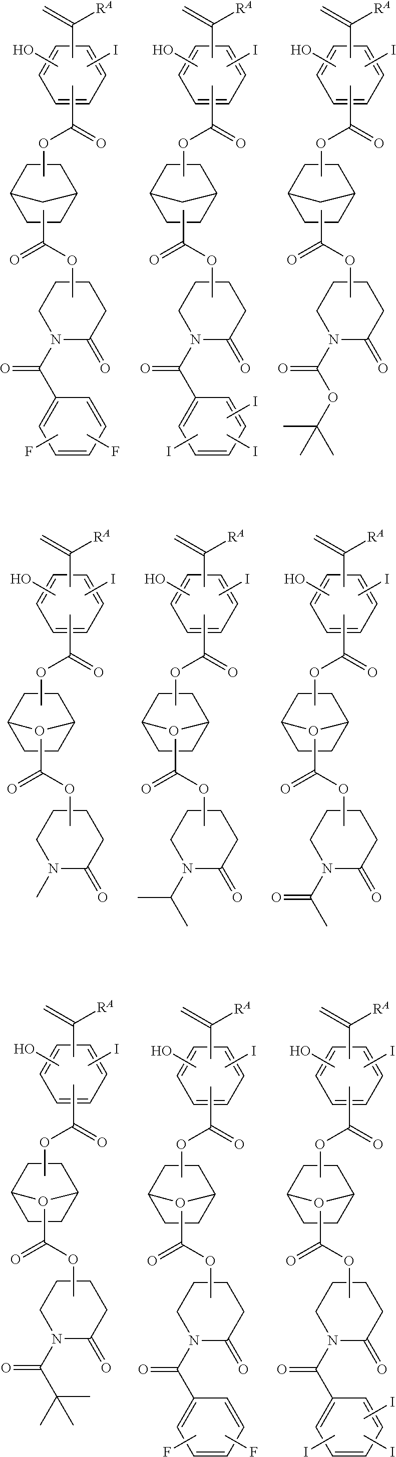

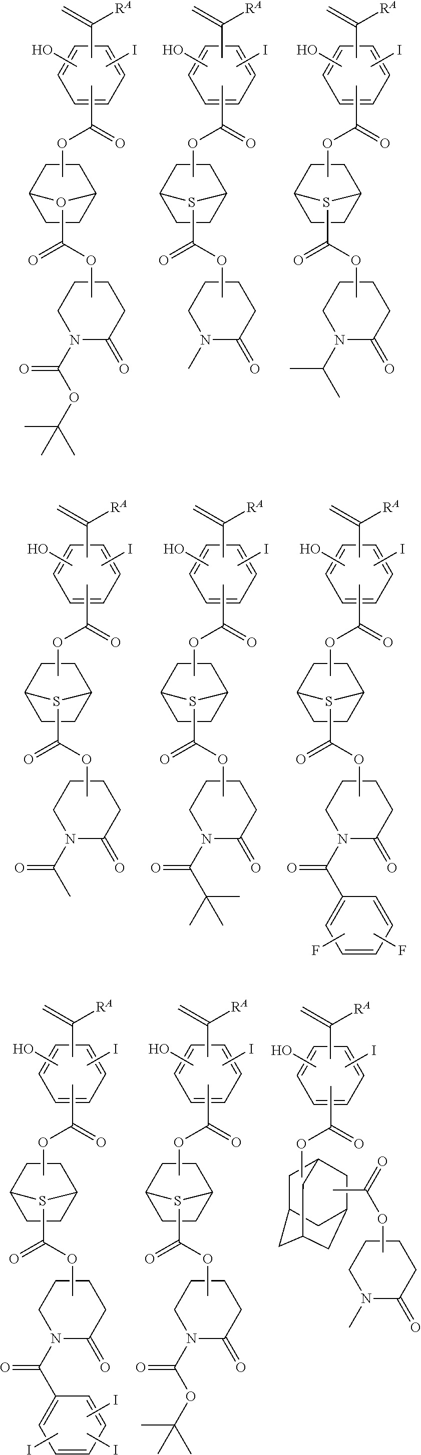

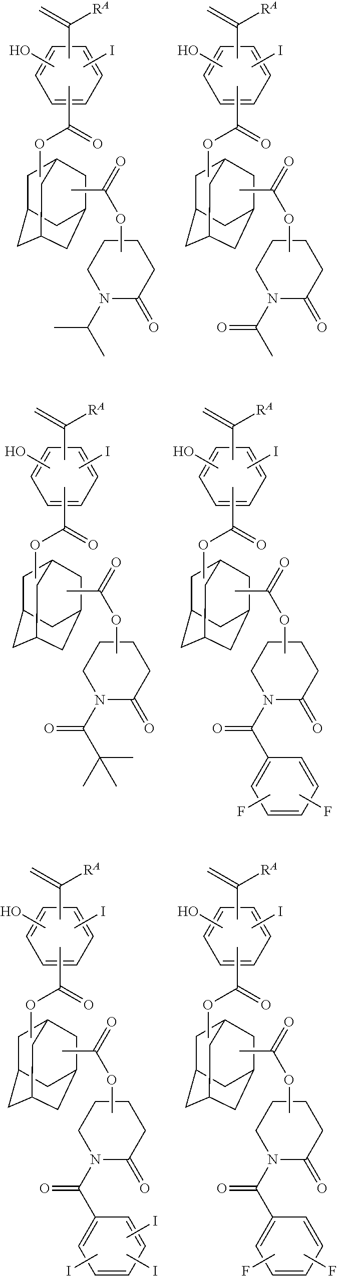

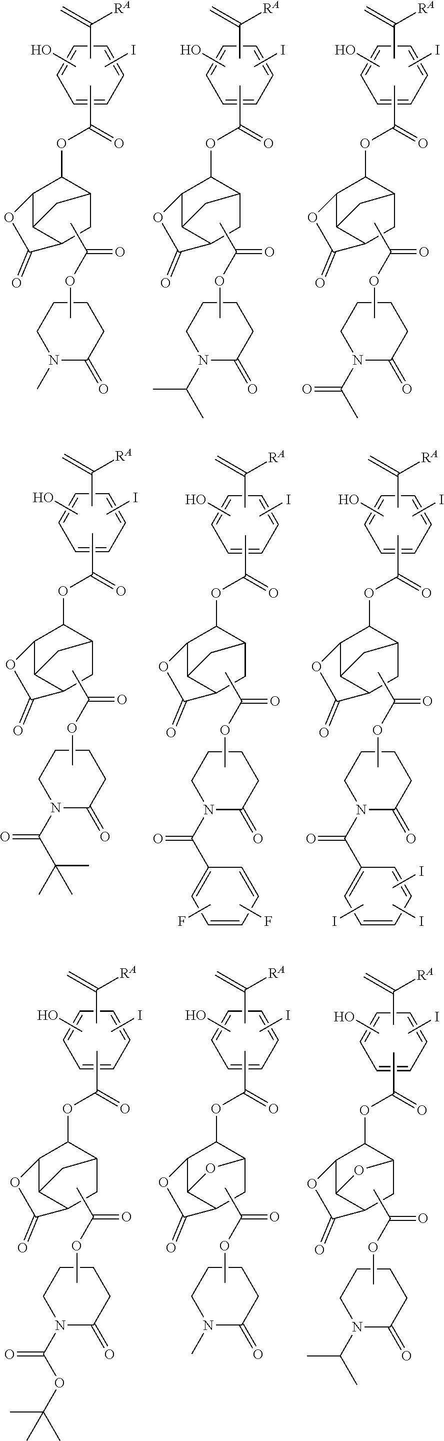

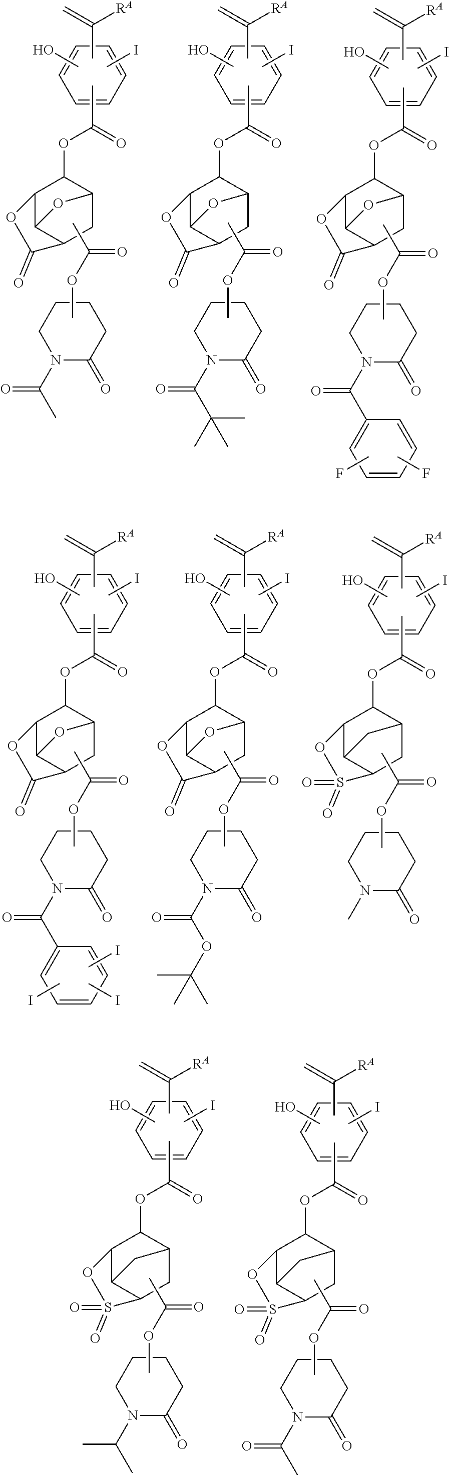

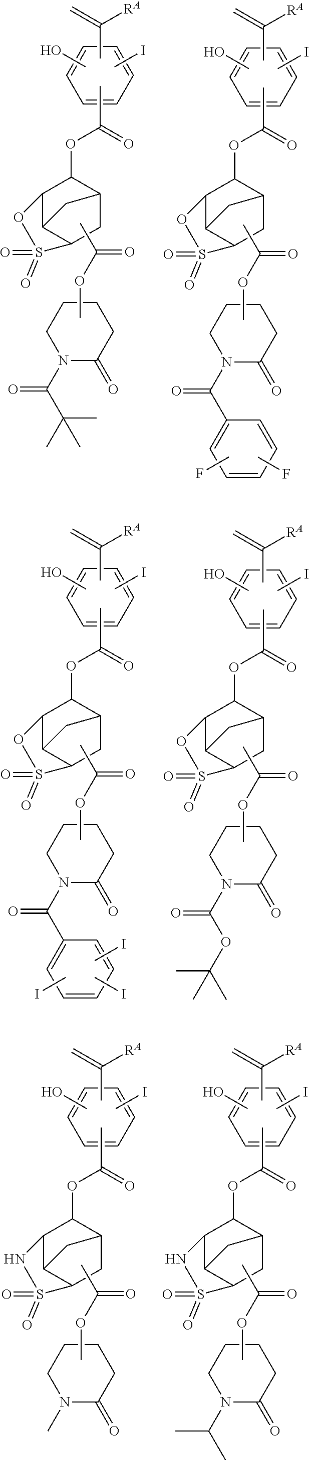

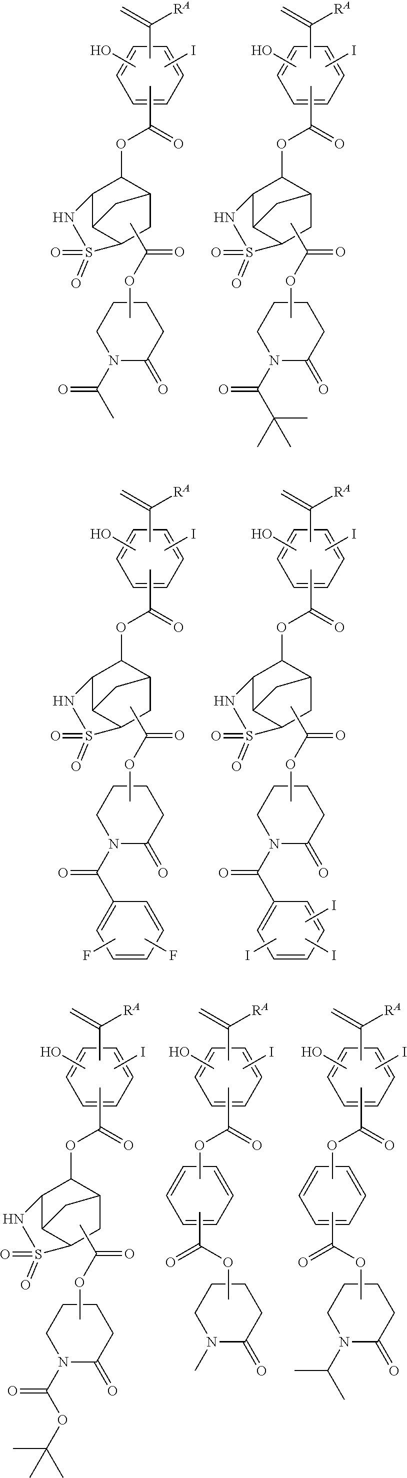

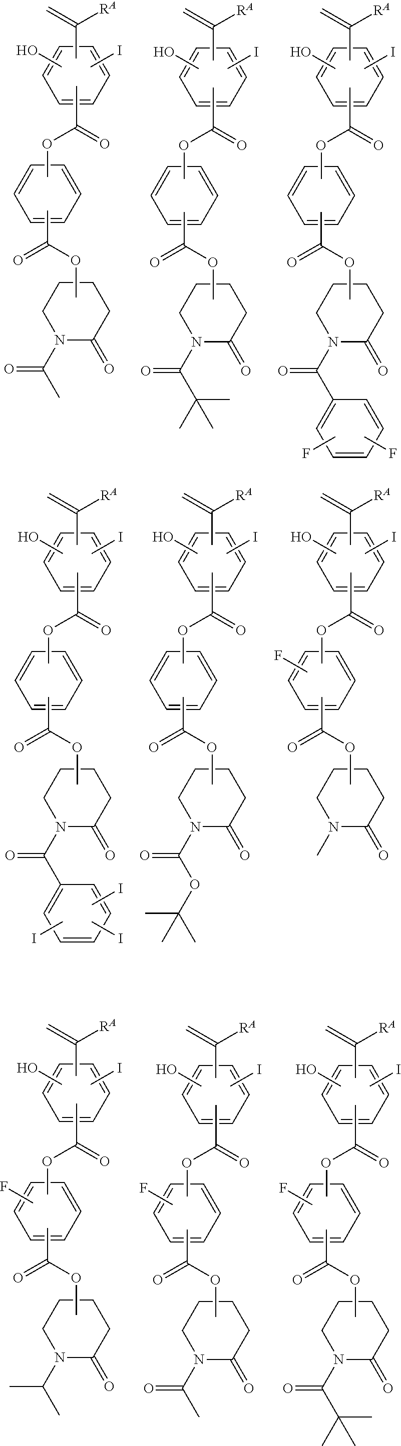

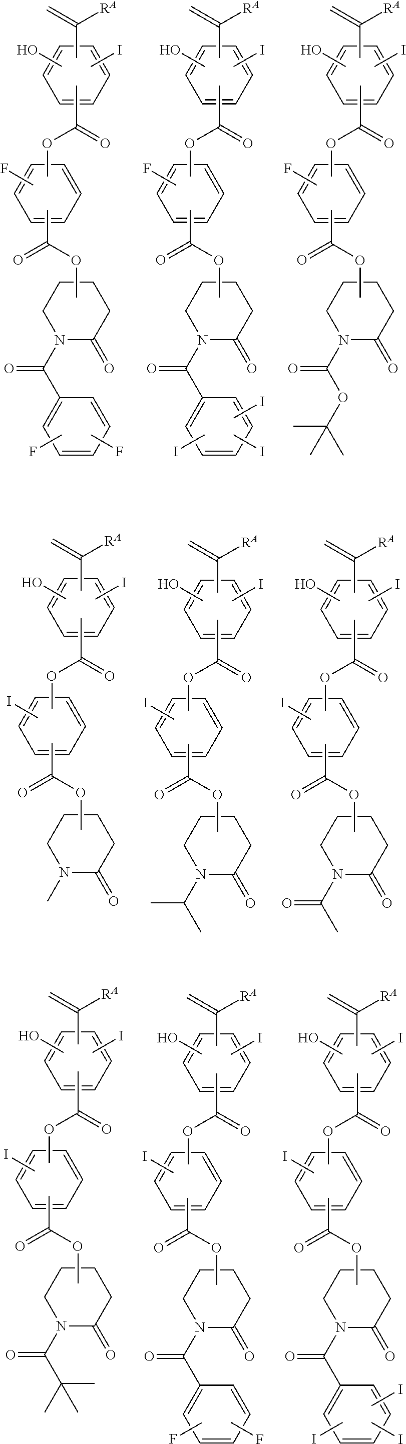

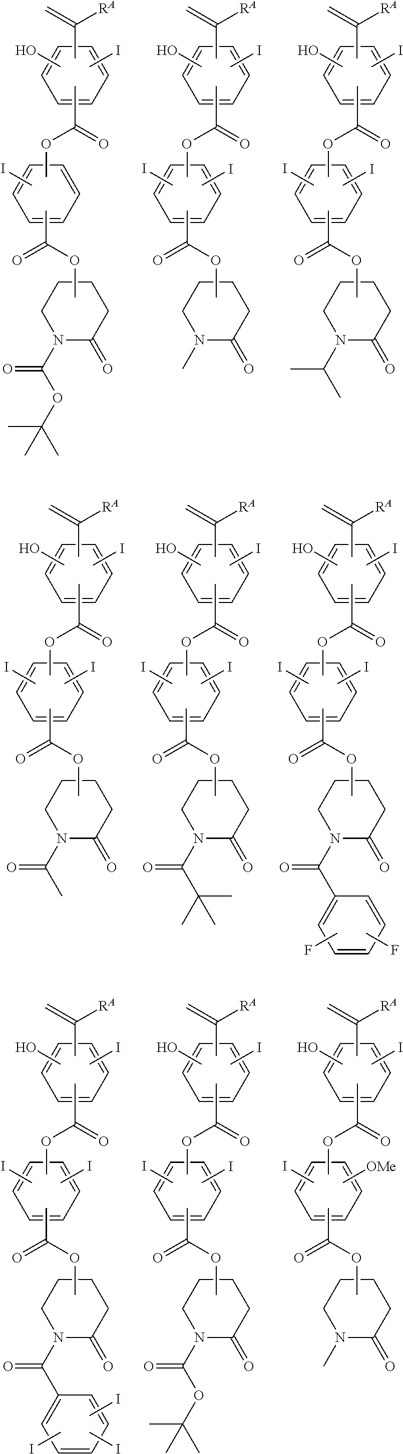

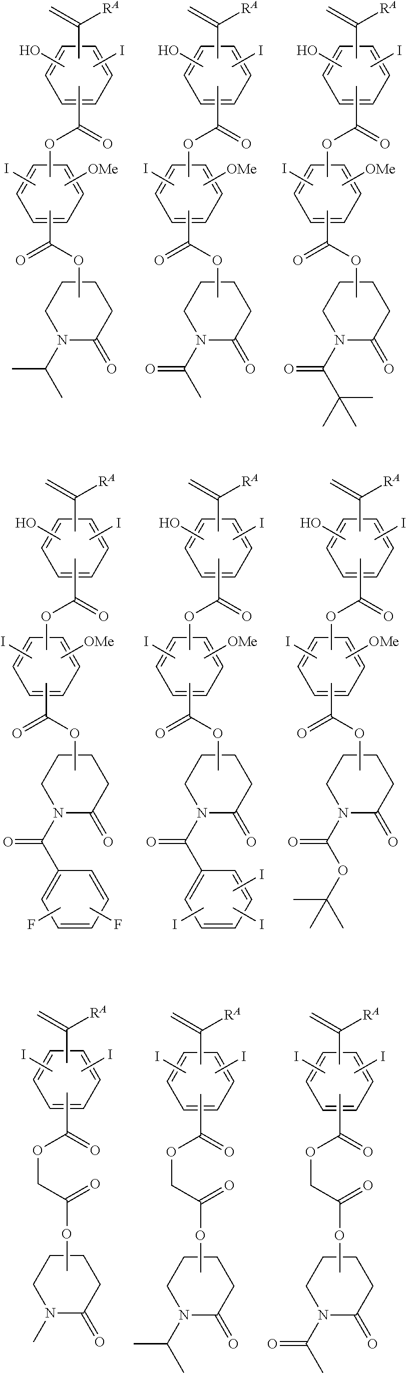

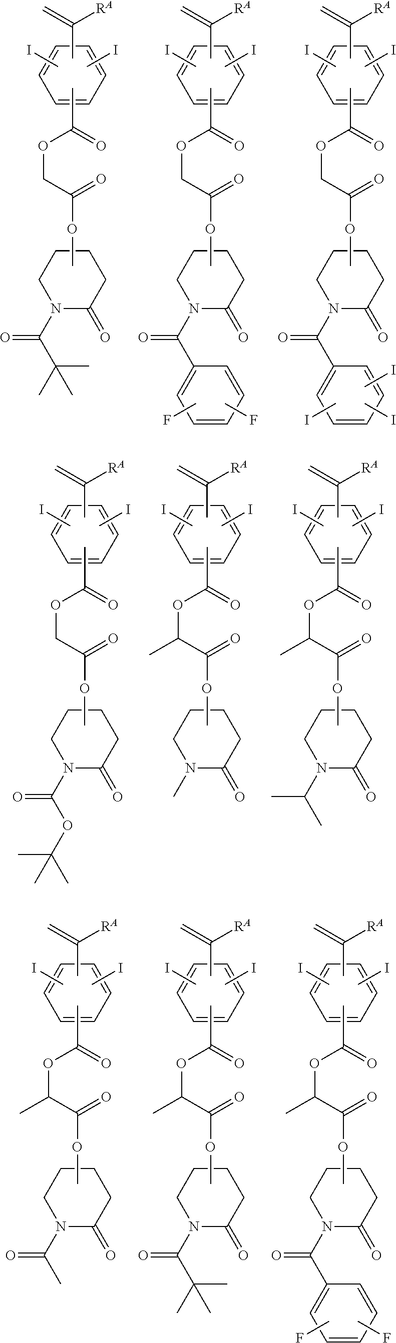

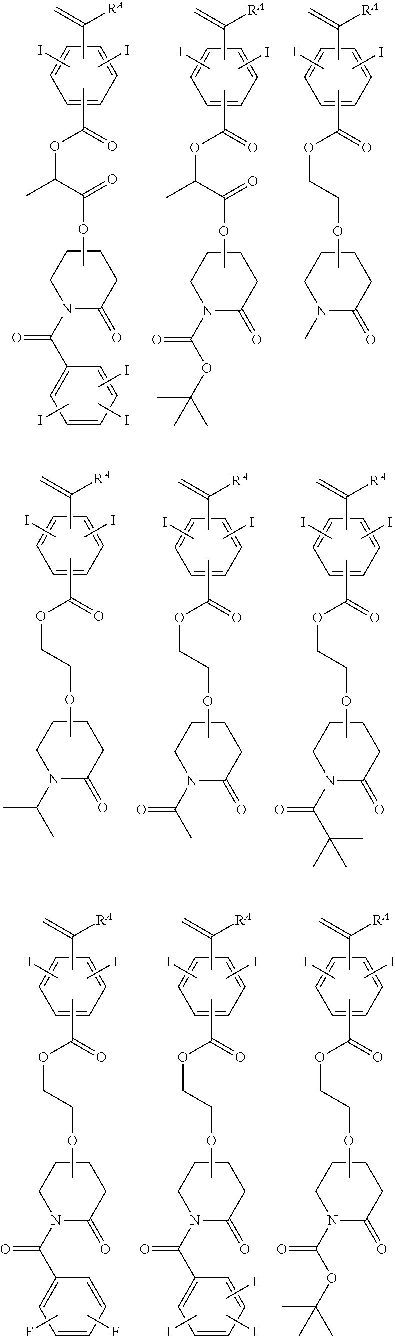

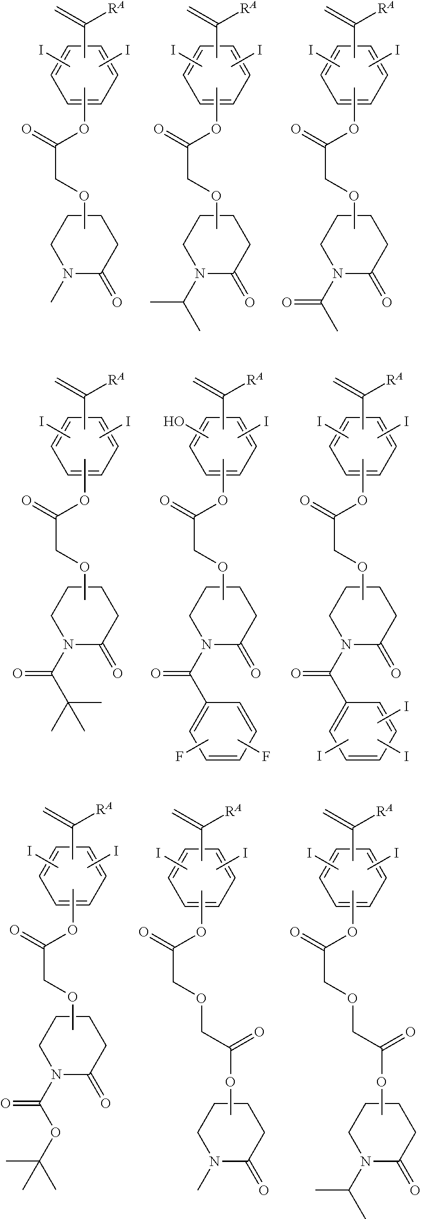

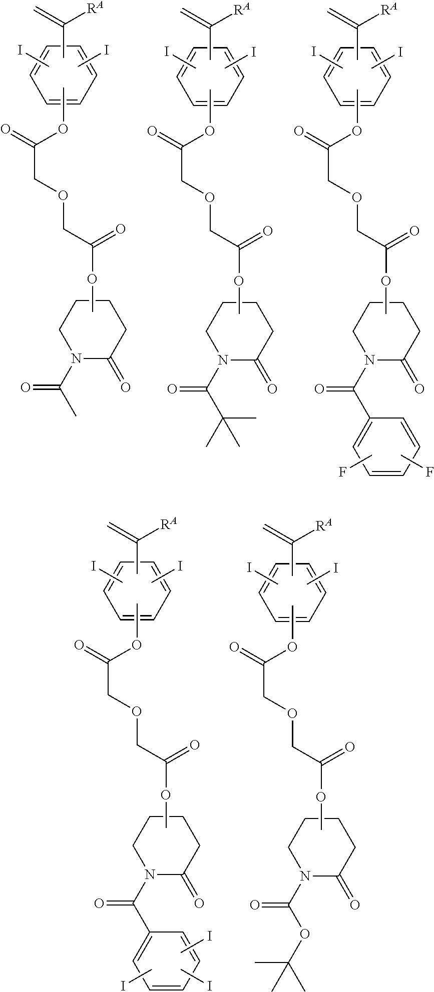

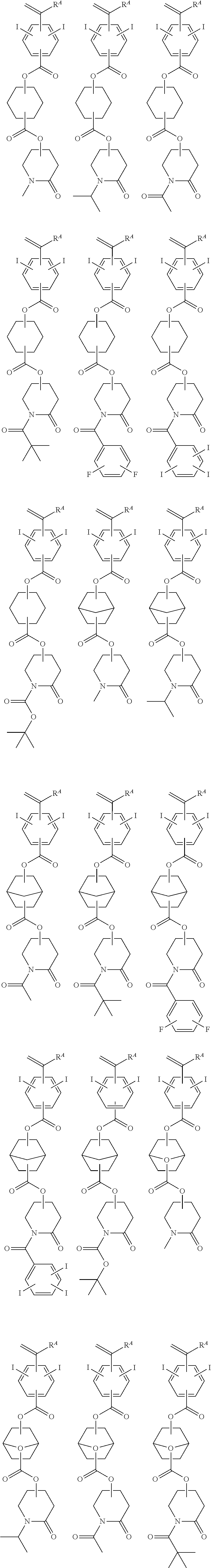

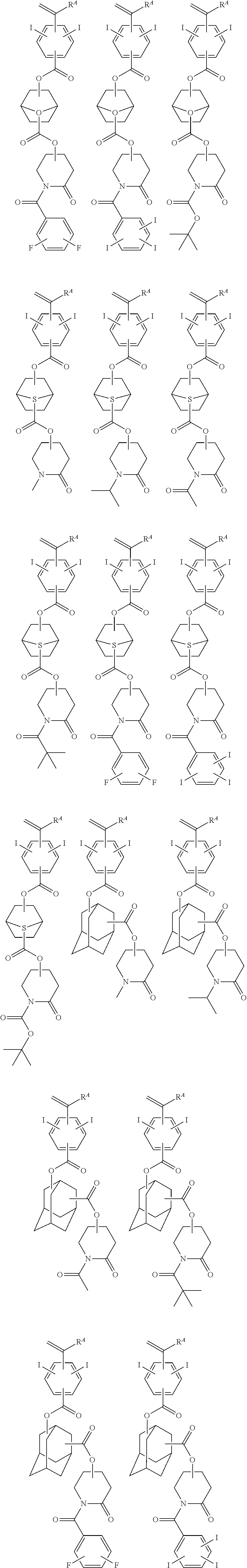

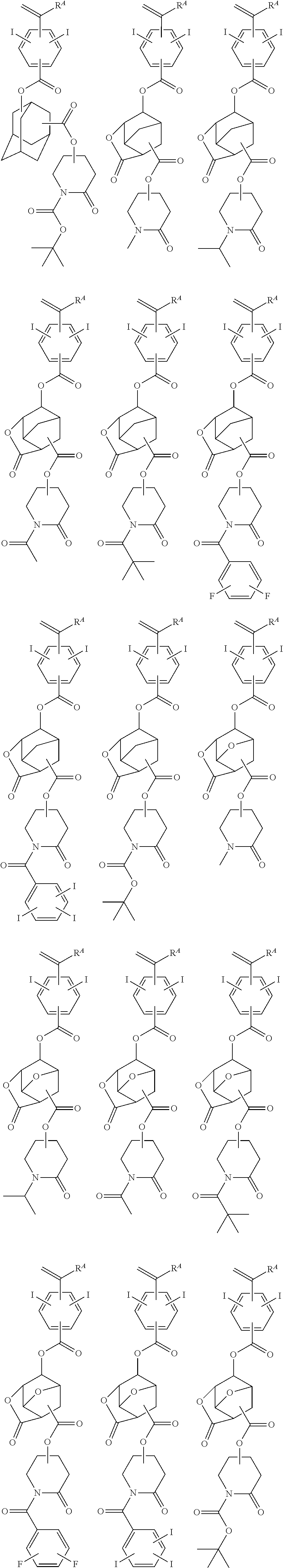

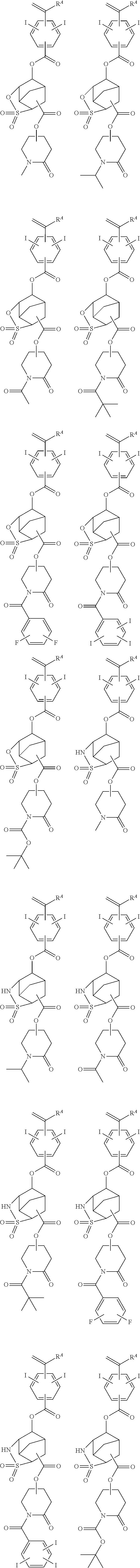

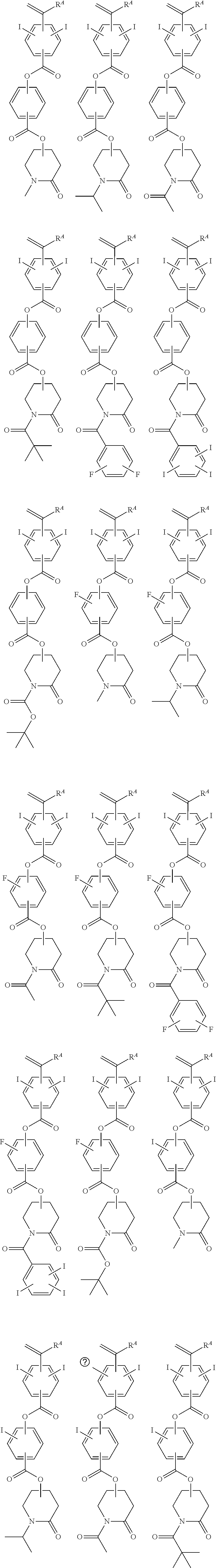

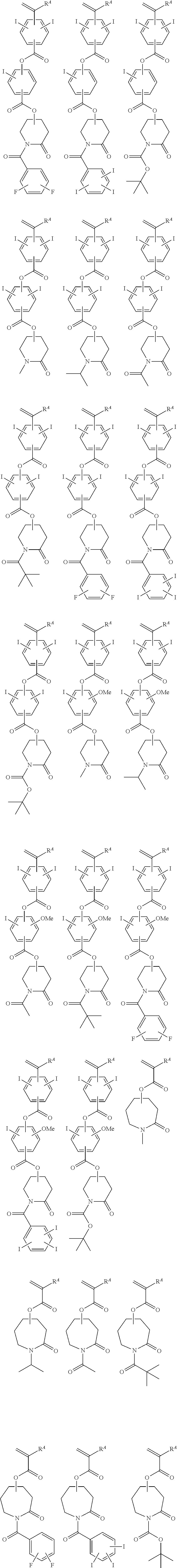

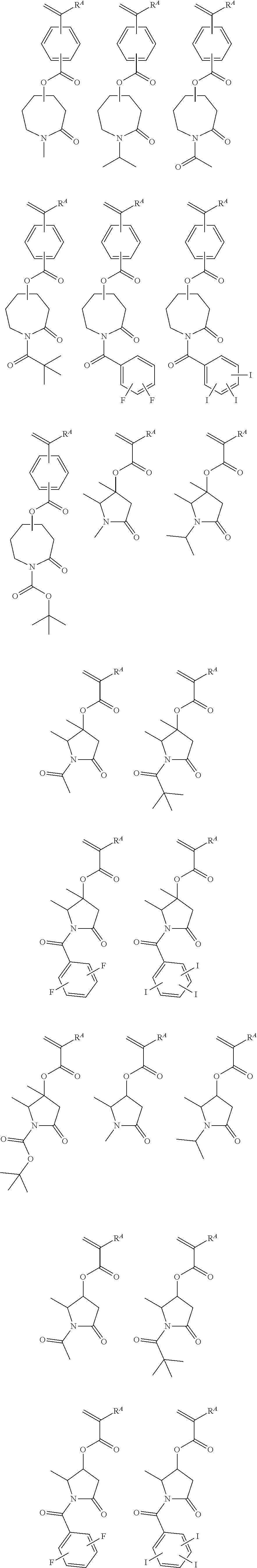

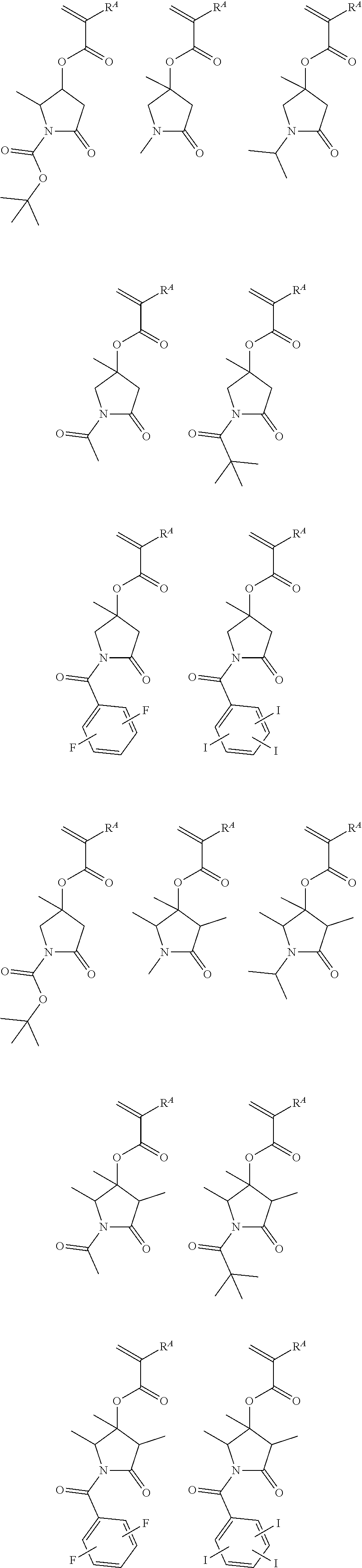

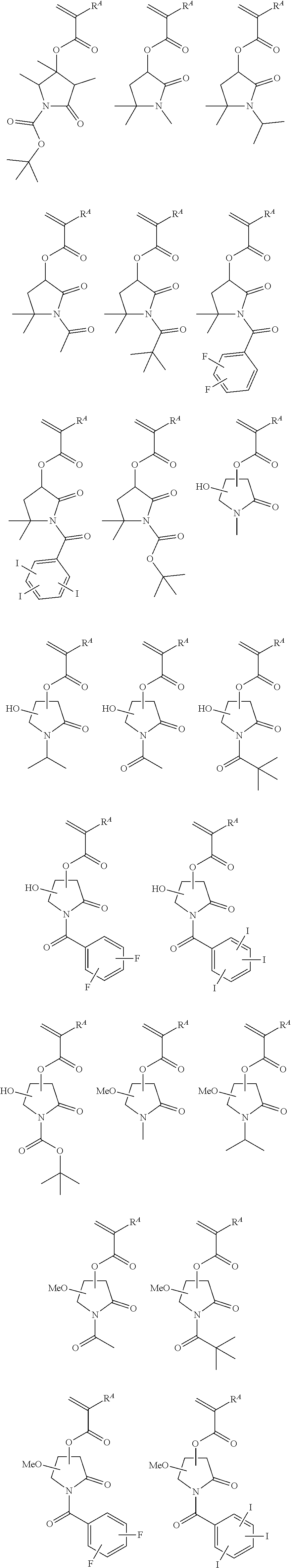

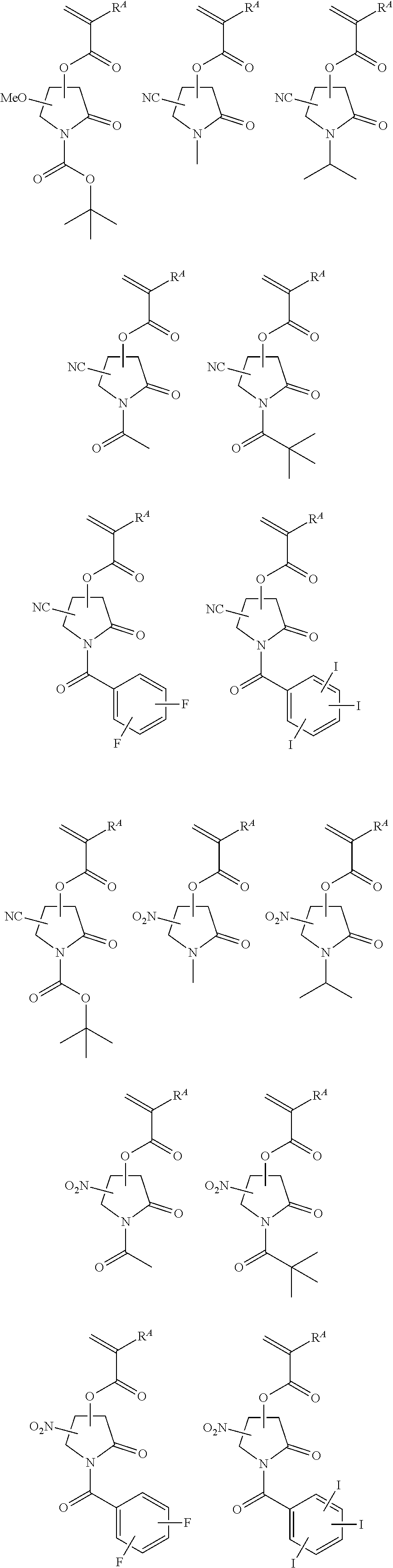

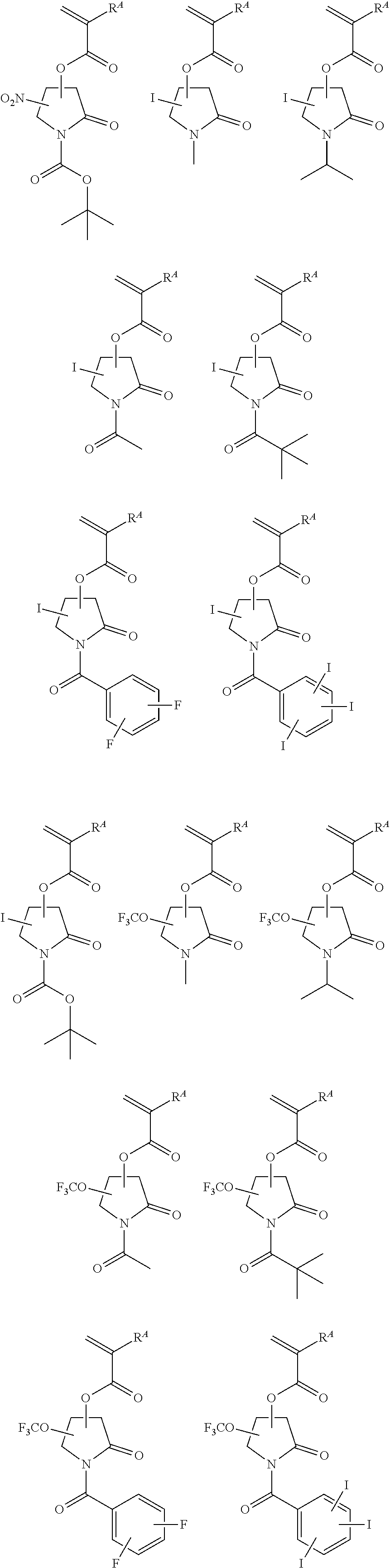

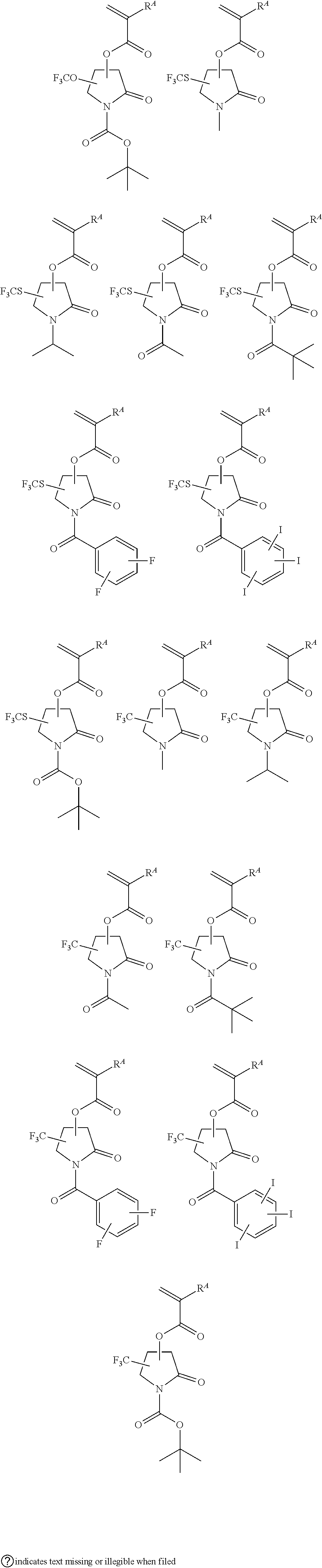

The monomer can be synthesized by any well-known methods. For the synthesis of a monocyclic lactam intermediate, the method described in Tetrahedron: Asymmetry (2003), 14 (22), 3541-3551 may be used. For the subsequent conversion to the monomer, any well-known organic chemical reaction may be used.

The above-mentioned preparation method is merely exemplary and the method of preparing the inventive monomer is not limited thereto.

The inventive monomer is structurally characterized by having a 5 to 7-membered monocyclic lactam structure. Since the monocycle has a smaller excluded volume than the fused ring, the polymer chain can be more dense. In general, the lactam ring has a higher polarity, lacks basicity, but due to the nitrogen atom contained, forms a hydrogen bond with the acid (or proton) generated by a photoacid generator for thereby restraining excessive acid diffusion. In an embodiment wherein an imide bond is formed by introducing an acyl group on the nitrogen atom, two carbonyl groups exert a chelating effect, enabling further suppression of acid diffusion. Due to the high polarity, the substrate adhesion is so strong that when the exposed resist film is developed in a developer, the resist pattern is prevented from collapsing. By the synergy of these effects, the inventive monomer enables to provide both full suppression of acid diffusion and substrate adhesion by virtue of monocyclic lactam and form a resist pattern having reduced LWR of line patterns, improved CDU of hole patterns, and high collapse resistance. The monomer is thus suited for formulating a positive resist composition.

### [Polymer]

Another embodiment of the invention is a polymer comprising repeat units derived from the monomer having formula (A), also referred to as repeat units (A), hereinafter. The repeat unit (A) is represented by the formula (Aa).

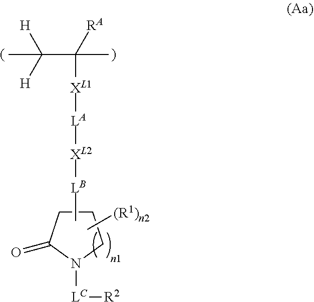

Herein, n1, n2, R^{A}, X^{L1}, X^{L2}, L^{A}, L^{B}, L^{C}, R¹ and R² are as defined above.

The repeat units (A) may be used alone or in admixture of two or more as constituent units of the base polymer.

The polymer may further comprise repeat units having the formula (a1) or repeat units having the formula (a2), also referred to as repeat units (a1) or (a2), hereinafter.

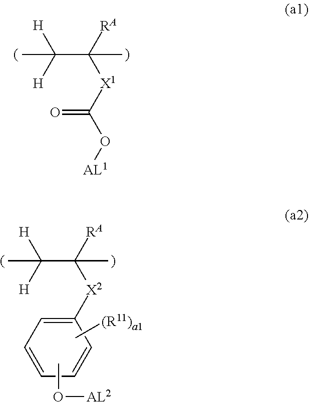

In formulae (a1) and (a2), R^{A} is each independently hydrogen, fluorine, methyl or trifluoromethyl.

In formula (a1), X¹ is a single bond, phenylene, naphthylene, *—C(═O)—O—X¹¹— or *—C(═O)—NH—X¹¹— wherein * designates a point of attachment to the carbon atom in the backbone. The phenylene or naphthylene group may be substituted with hydroxy, nitro, cyano, optionally fluorinated C₁-C₁₀ saturated hydrocarbyl moiety, optionally fluorinated C₁-C₁₀ saturated hydrocarbyloxy moiety, or halogen. X¹¹ is a C₁-C₁₀ saturated hydrocarbylene group, phenylene group or naphthylene group. The saturated hydrocarbylene group may contain hydroxy, ether bond, ester bond or lactone ring.

In formula (a2), X² is a single bond, *—C(═O)—O— or *—C(═O)—NH— wherein * designates a point of attachment to the carbon atom in the backbone.

In formula (a2), R¹¹ is halogen, cyano, hydroxy, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom.

In formula (a2), a1 is 0, 1, 2, 3 or 4, preferably 0 or 1. When a1 is 2, 3 or 4, a plurality of R¹¹ may be identical or different.

In formulae (a1) and (a2), AL¹ and AL² are each independently an acid labile group.

The acid labile group may be selected from a variety of such groups, for example, the groups described in U.S. Pat. No. 8,574,817 (JP-A 2013-080033) and U.S. Pat. No. 8,846,303 (JP-A 2013-083821), but are not limited thereto.

Typical of the acid labile group are groups having the following formulae (AL-1) to (AL-3).

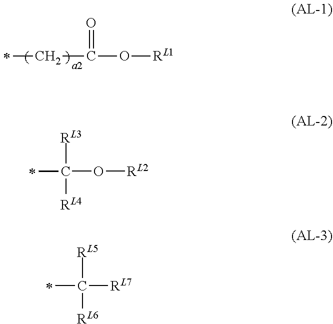

In formulae (AL-1) and (AL-2), R^{L1} and R^{L2} are each independently a C₁-C₄₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₂₀ hydrocarbyl groups are preferred.

In formula (AL-1), a2 is an integer of 0 to 10, preferably 1, 2, 3, 4 or 5.

In formula (AL-2), R^{L3} and R^{L4} are each independently hydrogen or a C₁-C₂₀ hydrocarbyl group which may contain a heteroatom such as oxygen, sulfur, nitrogen or fluorine. The hydrocarbyl group may be saturated or unsaturated and straight, branched or cyclic. Any two of R^{L2}, R^{L3} and R^{L4} may bond together to form a C₃-C₂₀ ring with the carbon atom or carbon and oxygen atoms to which they are attached. Rings of 4 to 16 carbon atoms are preferred, with aliphatic rings being more preferred.

In formula (AL-3), R^{L5}, R^{L6} and R^{L7} are each independently a C₁-C₂₀ hydrocarbyl group which may contain a heteroatom such as oxygen, sulfur, nitrogen or fluorine. The hydrocarbyl group may be saturated or unsaturated and straight, branched or cyclic. Any two of R^{L5}, R^{L6} and R^{L7} may bond together to form a C₃-C₂₀ ring with the carbon atom to which they are attached. Rings of 4 to 16 carbon atoms are preferred, with aliphatic rings being more preferred.

Other examples of the acid labile group include those described in JP-A 2023-123222, paragraphs [0064]-[0068] and JP 7492842, paragraphs [0013]-[0014]. These acid labile groups utilize the conjugated olefins or acrylate derivatives formed after acid elimination reaction as the reaction driving force.

Examples of repeat units (a1) are shown below, but not limited thereto.

Herein R^{A} and AL¹ are as defined above.

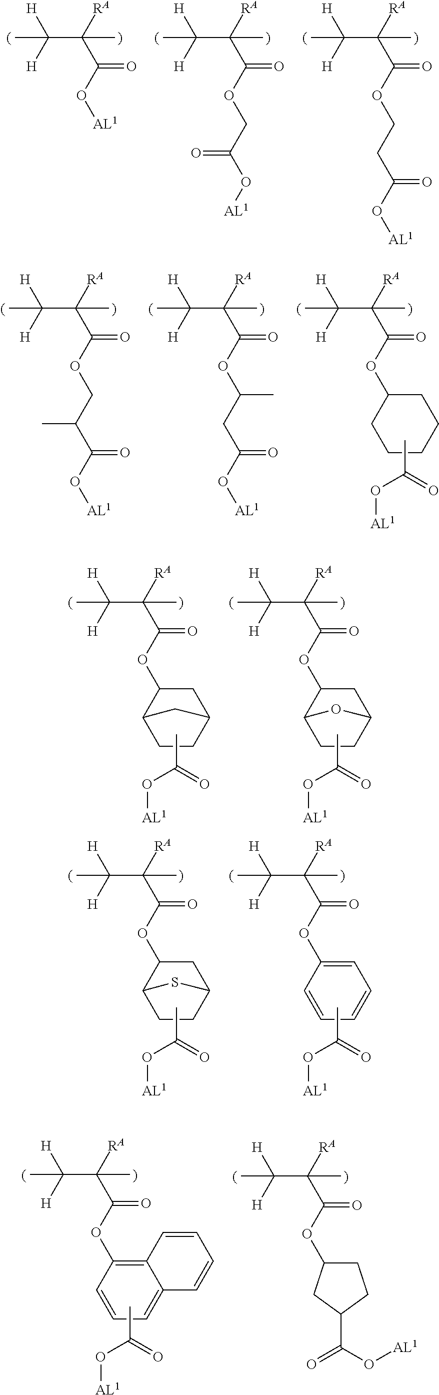

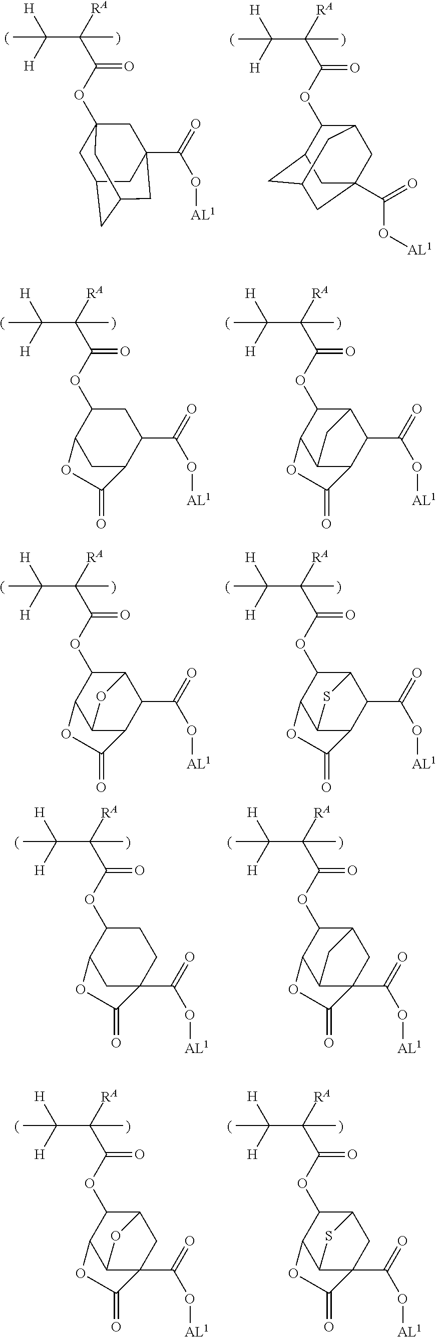

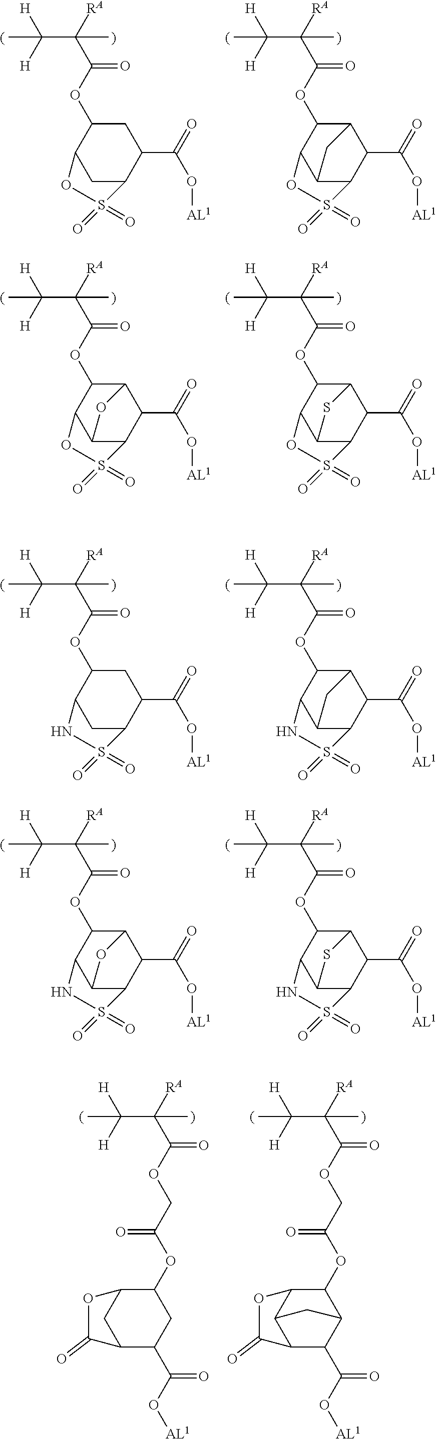

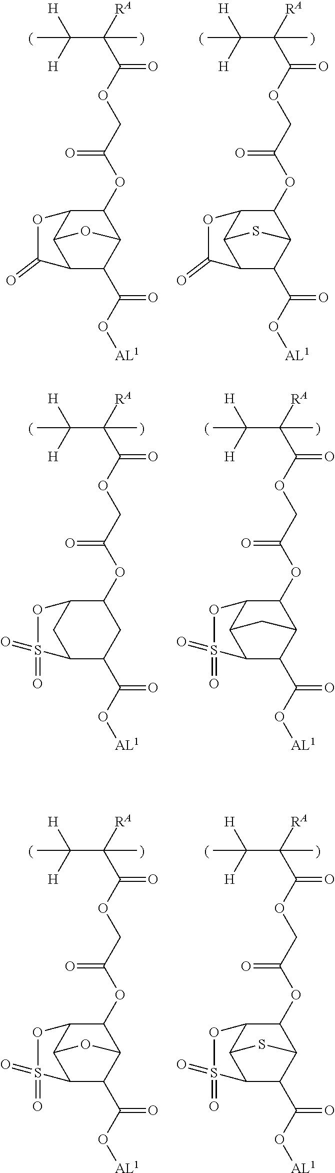

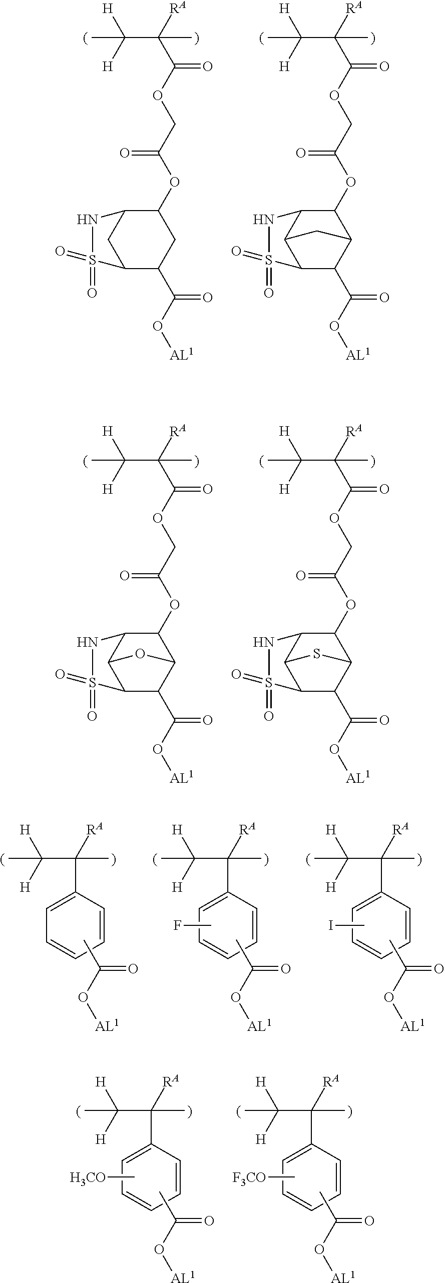

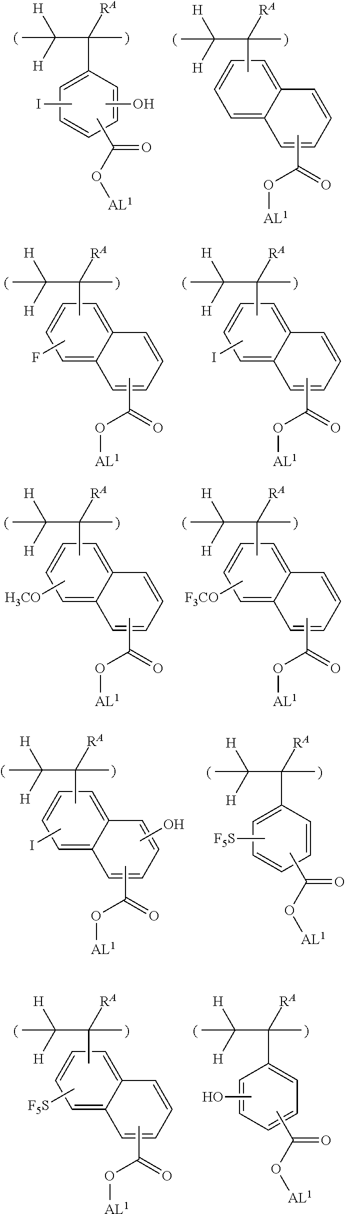

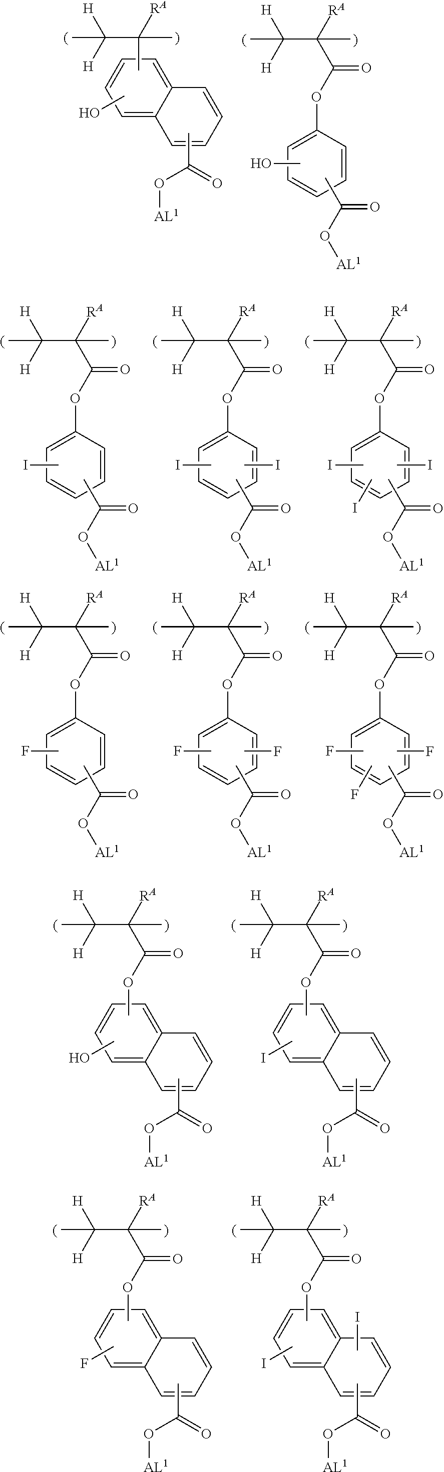

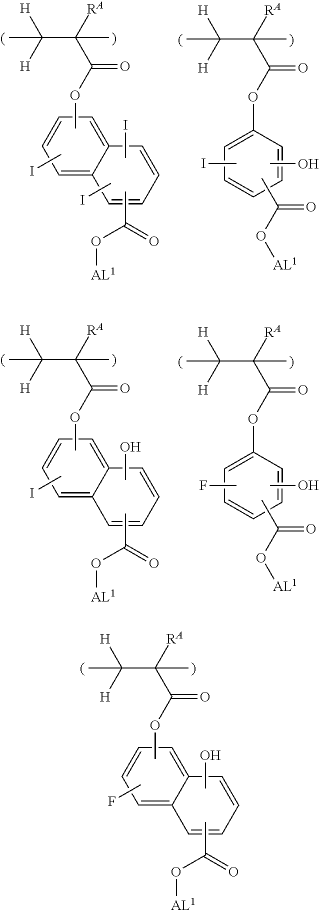

Examples of repeat units (a2) are shown below, but not limited thereto. Herein R^{A} and AL² are as defined above.

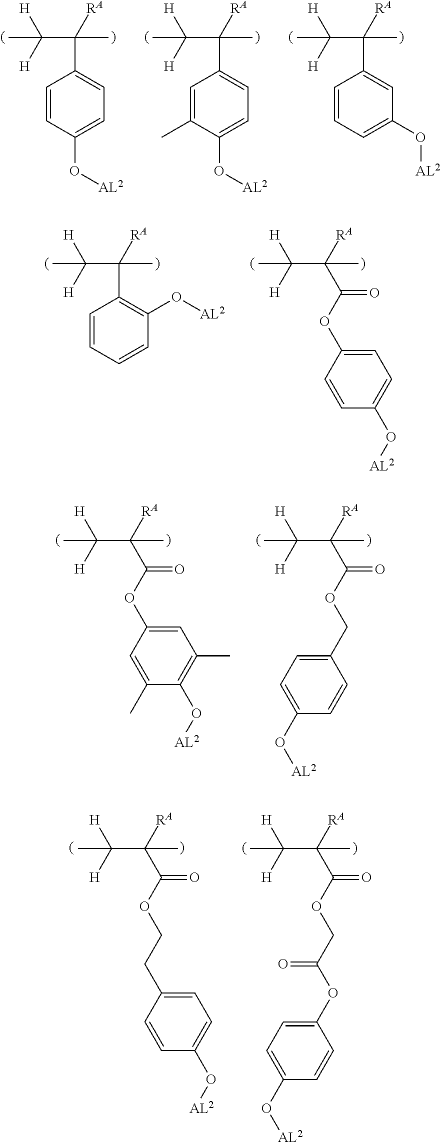

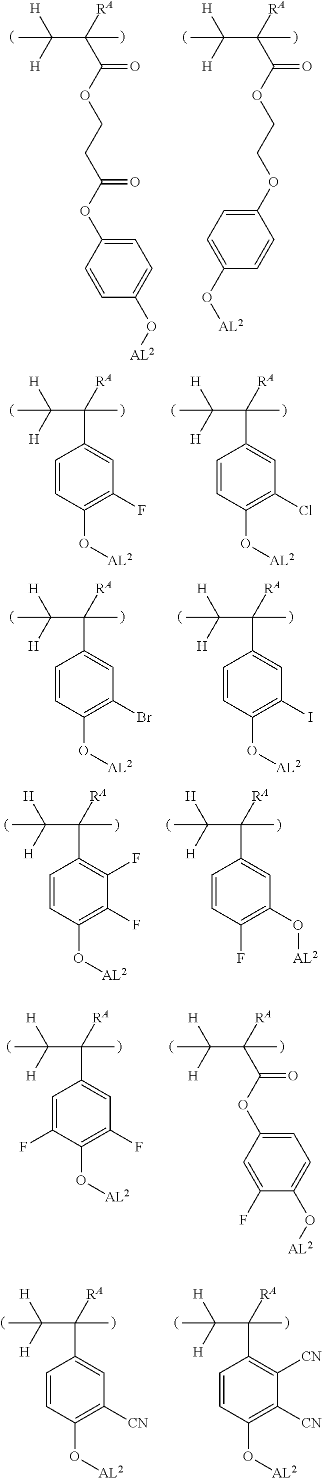

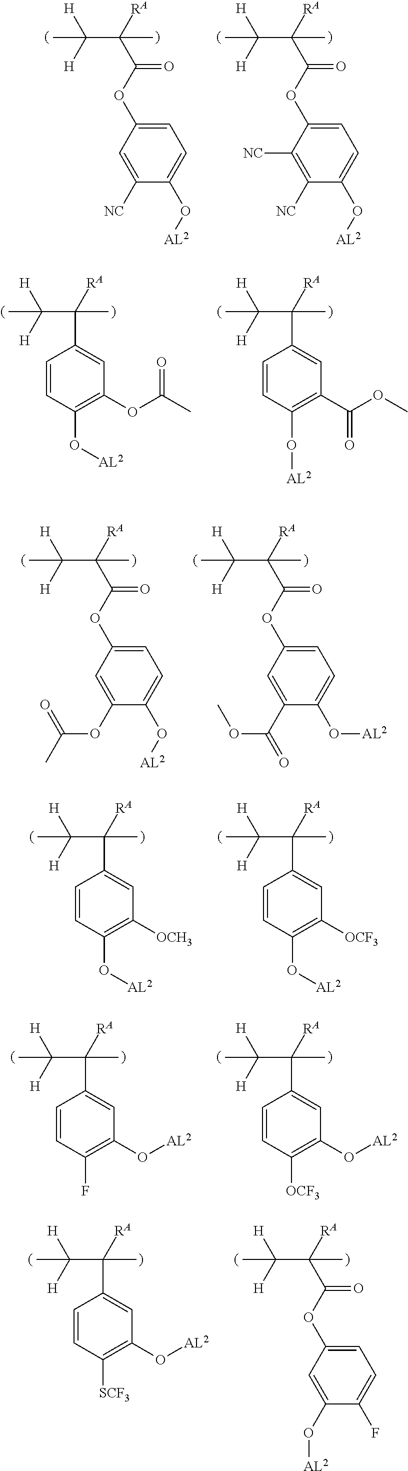

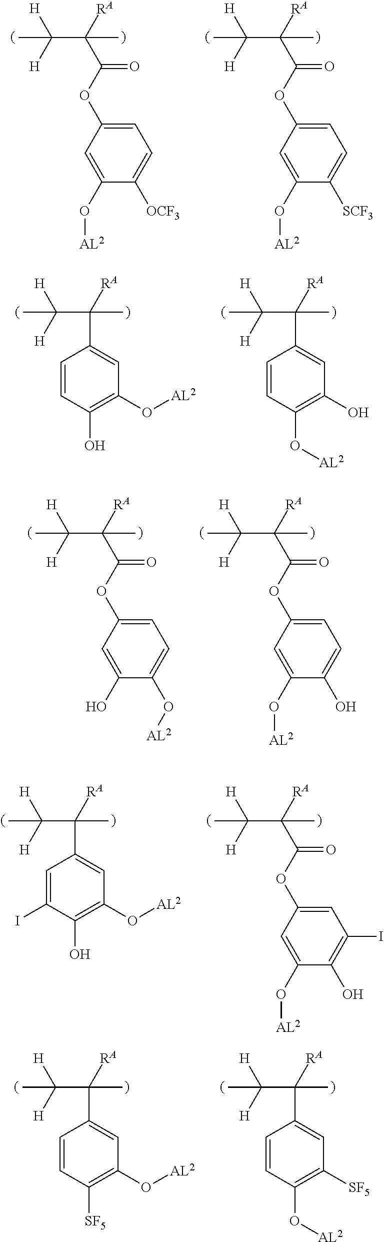

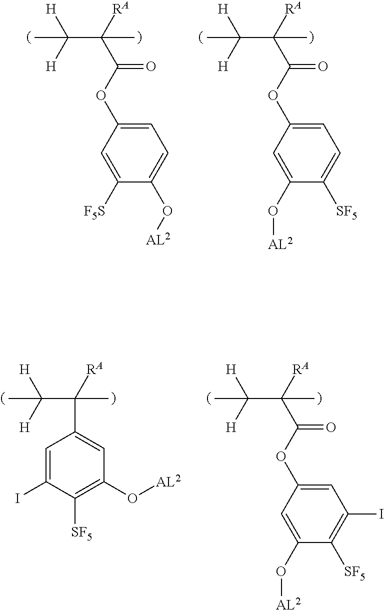

The polymer may further comprise repeat units having the formula (a3), also referred to as repeat units (a3).

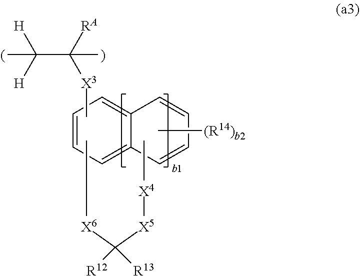

In formula (a3), b1 is 0 or 1. The relevant structure is a benzene ring when b1=0, and a naphthalene ring when b1=1. The benzene ring corresponding to b1=0 is preferred from the aspect of solvent solubility. The subscript b2 is 0, 1, 2 or 3 when b1=0, and b2 is 0, 1, 2, 3, 4 or 5 when b1=1. It is preferred for reactant availability that b2 be 0, 1, 2 or 3, more preferably 0, 1 or 2.

In formula (a3), R^{A} is hydrogen, fluorine, methyl or trifluoromethyl. Of these, hydrogen and methyl are preferred, with hydrogen being most preferred.

In formula (a3), X³ is a single bond, *—C(═O)—O— or *—C(═O)—NH—, wherein * designates a point of attachment to the carbon atom in the backbone. X³ is preferably a single bond or *—C(═O)—O—, with a single bond being more preferred.

In formula (a3), X⁴ is a single bond, C₁-C₄ aliphatic hydrocarbylene group, carbonyl, sulfonyl or a group obtained by combining the foregoing. It is preferred for reactant availability that X⁴ be a single bond, carbonyl or sulfonyl. It is more preferred from the aspect of a polar group created after reaction that X⁴ be a single bond or carbonyl.

In formula (a3), X⁵ and X⁶ are each independently oxygen or sulfur. X⁴ and X⁶ are attached to adjoining carbon atoms on the aromatic ring. X⁵ and X⁶ may be identical or different. It is preferred from the aspect of reactivity that both X⁵ and X⁶ be oxygen.

In formula (a3), R¹² and R¹³ are each independently hydrogen or a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl and 2-phenylethyl; and combinations thereof. In the hydrocarbyl group, some or all of the hydrogen atoms may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonate ester bond, carbonate bond, lactone ring, sultone ring, carboxylic anhydride (—C(═O)—O—C(═O)—) or haloalkyl moiety.

Also, R¹² and R¹³ may bond together to form a ring with the carbon atom to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

In formula (a3), R¹⁴ is halogen, hydroxy, cyano, nitro, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{14A})(R^{14B}) R^{14A} and R^{14B} are each independently hydrogen or a C₁-C₆ hydrocarbyl group. Suitable halogen atoms include fluorine, chlorine, bromine and iodine, with fluorine and iodine being preferred. The hydrocarbyl group and hydrocarbyl moiety in the hydrocarbyloxy, hydrocarbyloxycarbonyl, and hydrocarbylthio groups may be saturated or unsaturated and straight, branched or cyclic. Examples thereof are as exemplified above for the hydrocarbyl groups R¹² and R¹³. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. When b2 is 2 or more, a plurality of R¹⁴ may be identical or different.

When b2 is 2 or more, a plurality of R¹⁴ may bond together to form a ring with carbon atoms in the aromatic ring to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonic ester bond, carbonate bond, lactone ring, sultone ring, carboxylic anhydride (—C(═O)—O—C(═O)—) or haloalkyl moiety.

Examples of repeat unit (a3) are shown below, but not limited thereto. Herein R^{A} is as defined above. The positions of attachment of substituent groups on the aromatic ring are interchangeable.

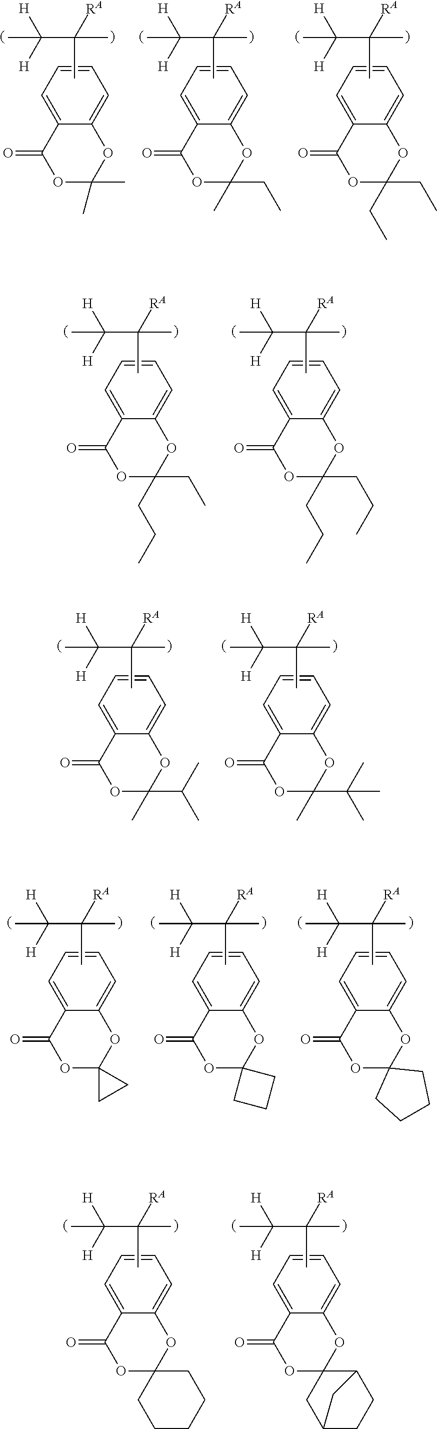

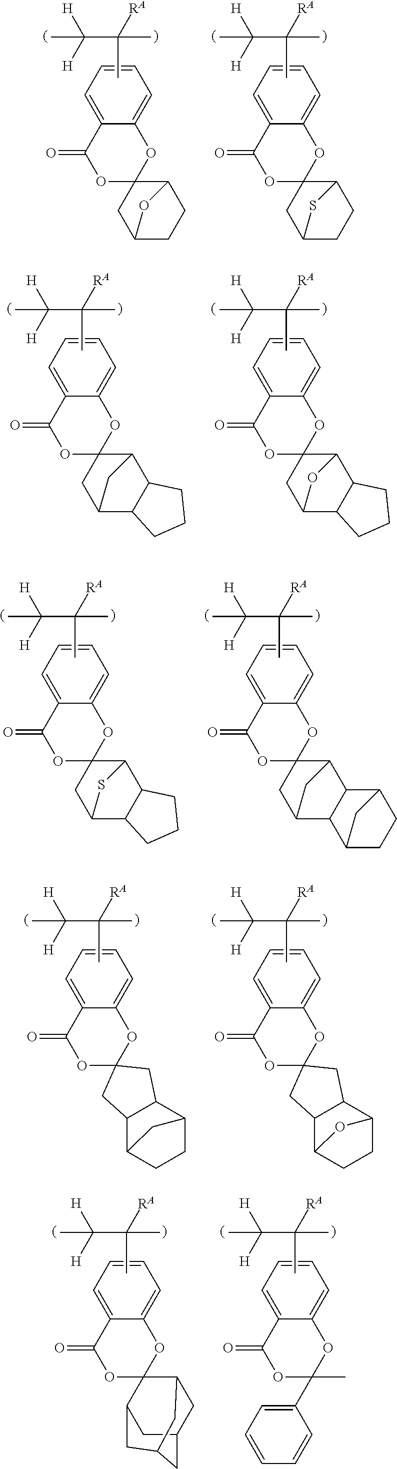

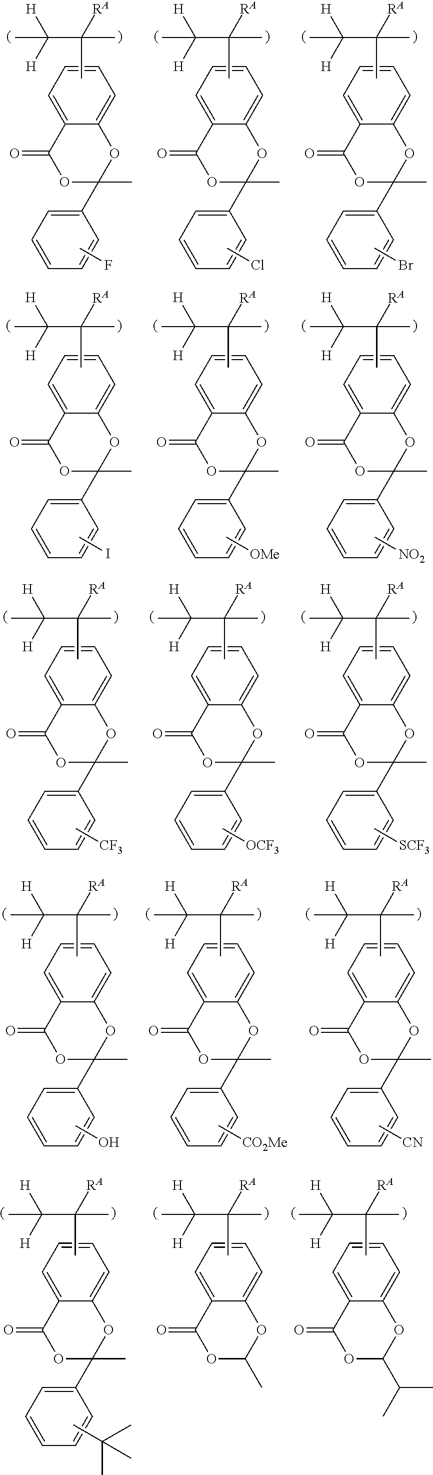

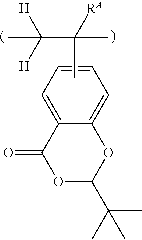

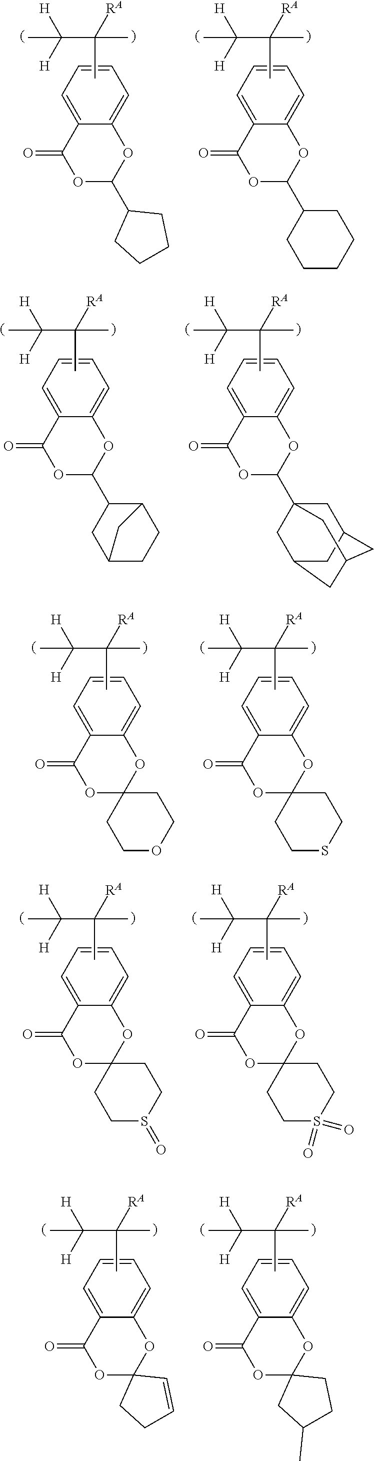

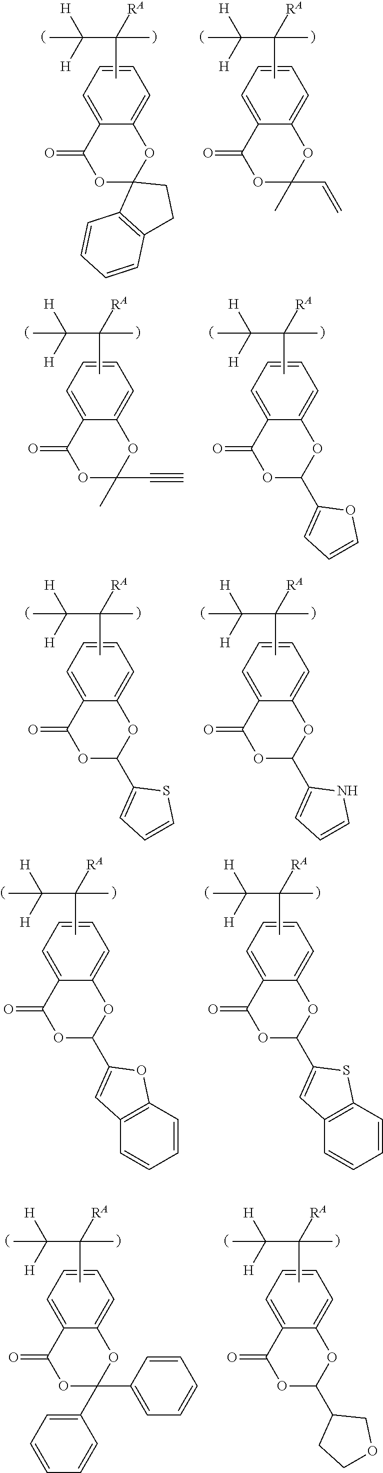

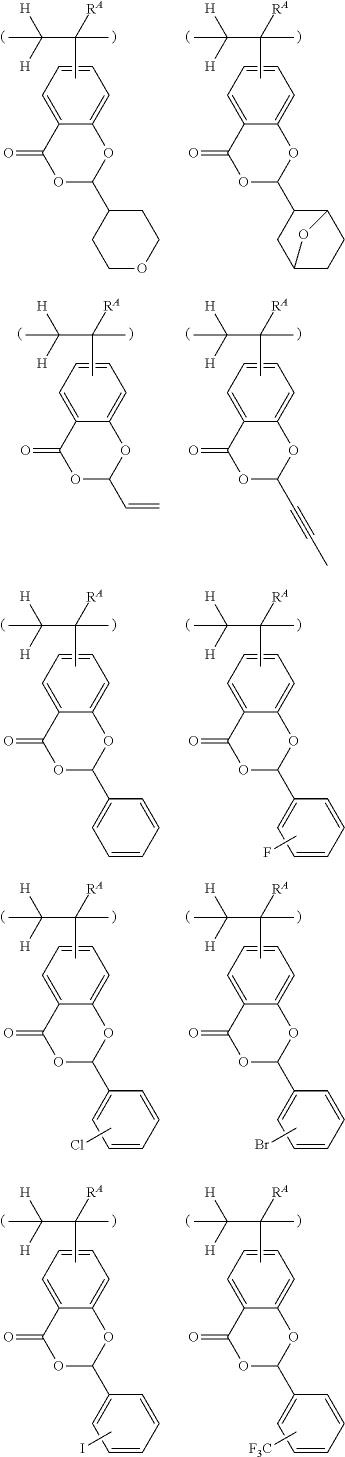

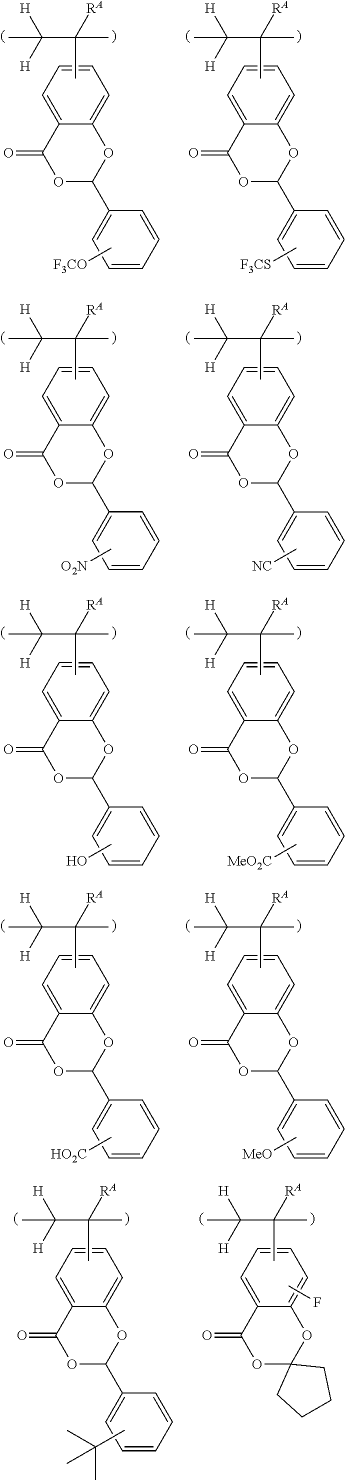

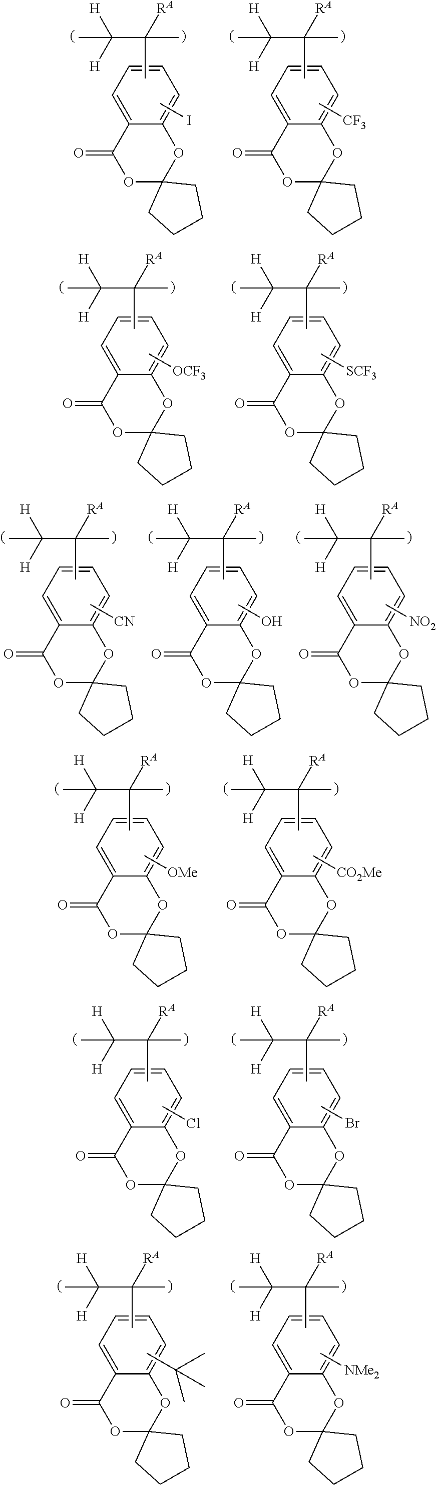

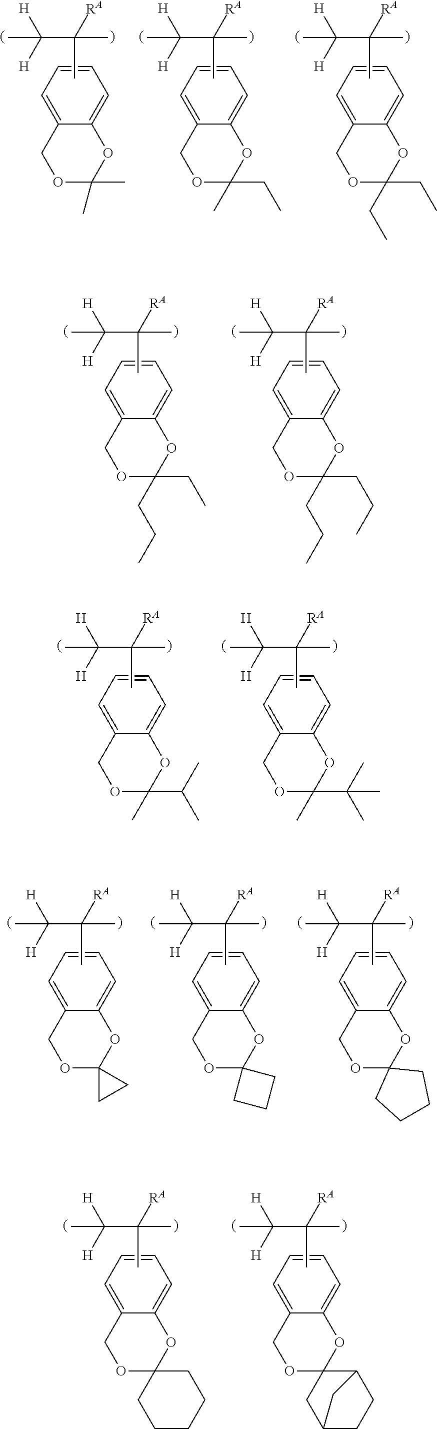

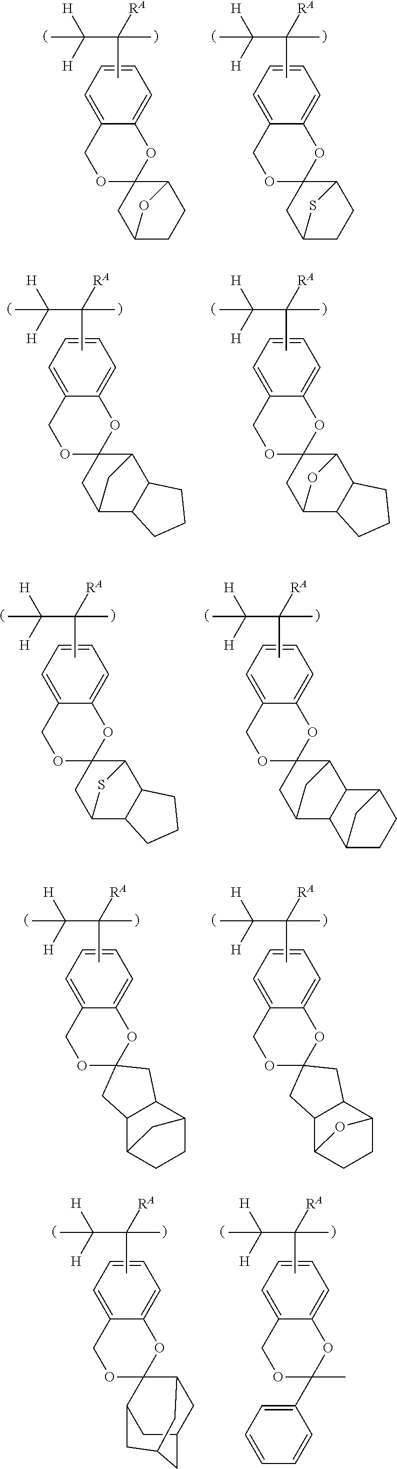

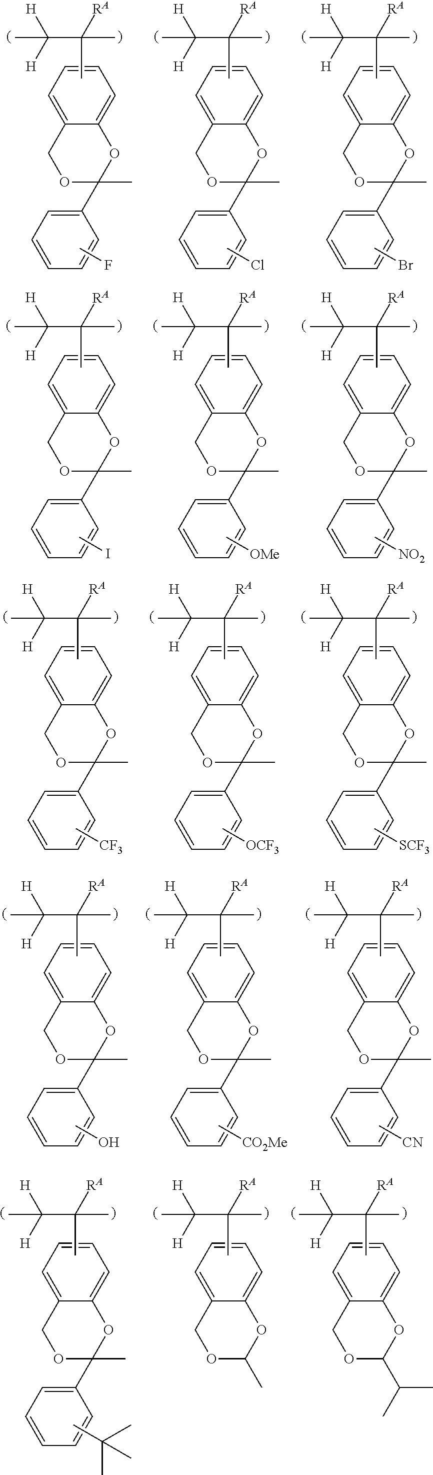

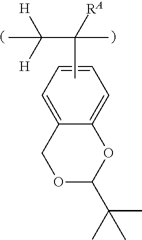

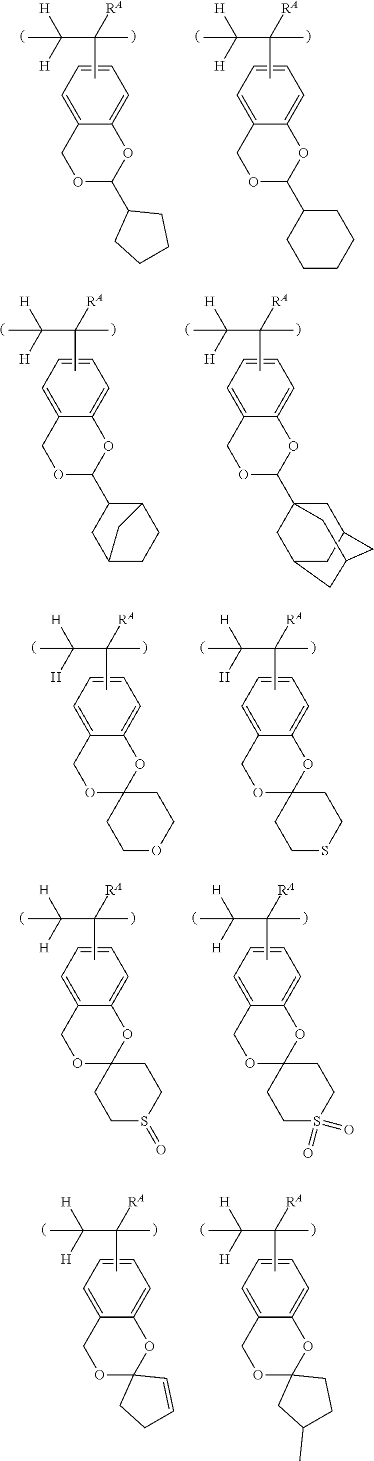

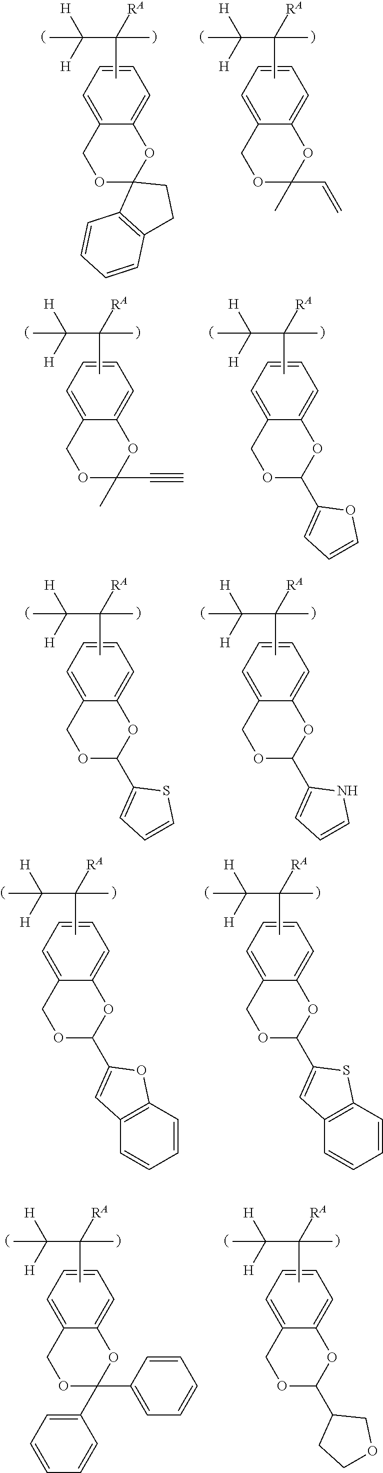

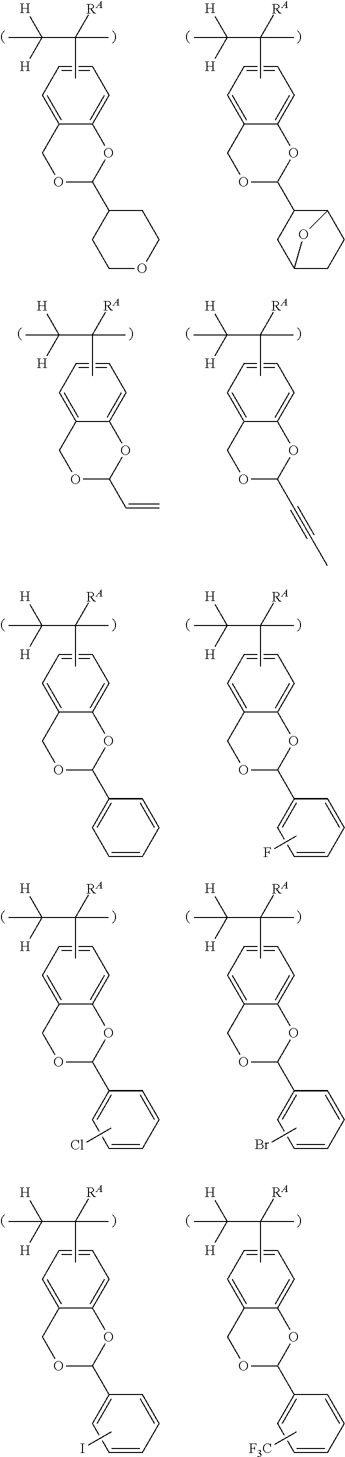

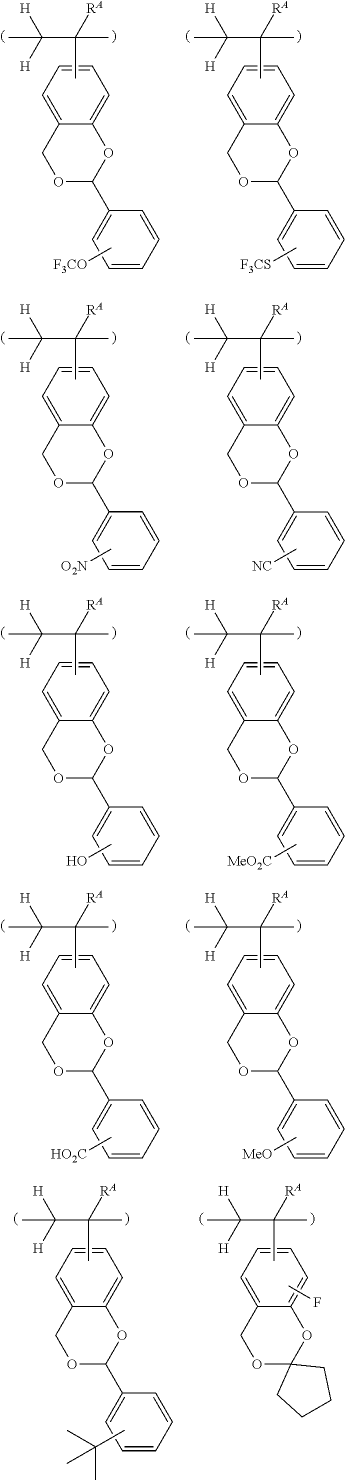

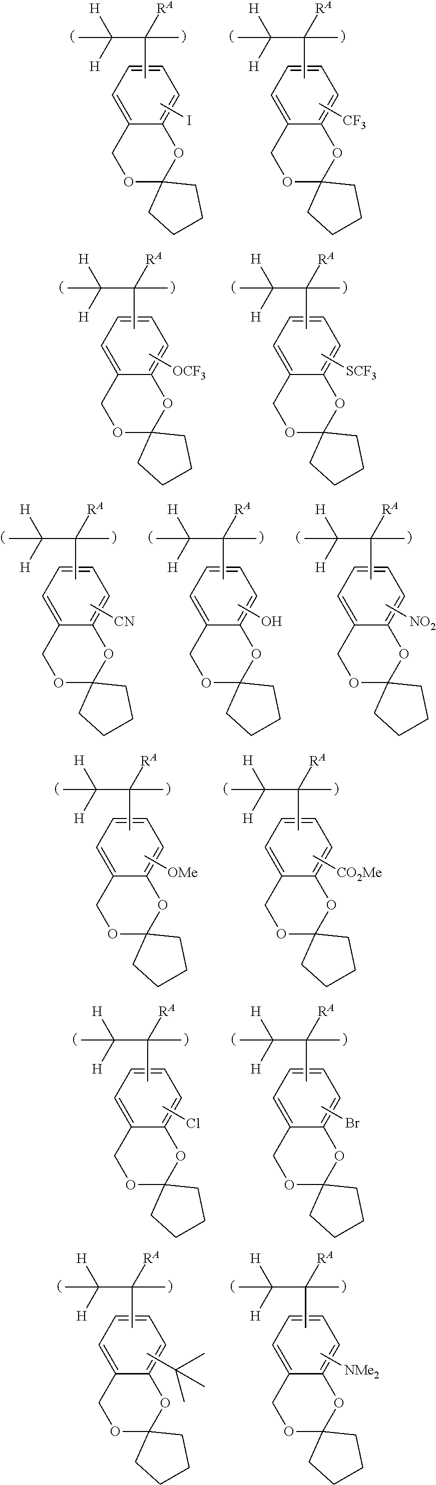

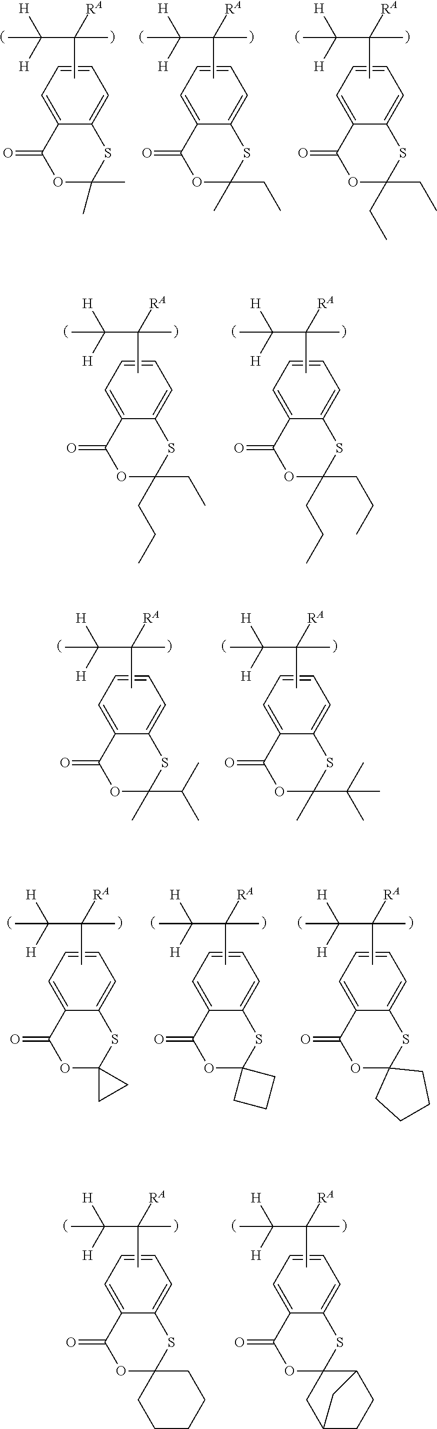

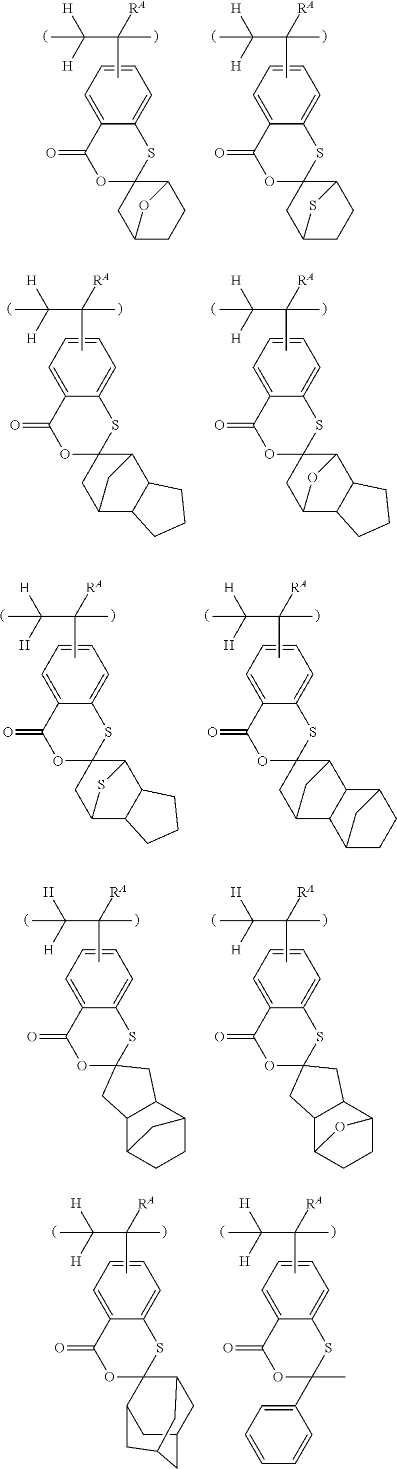

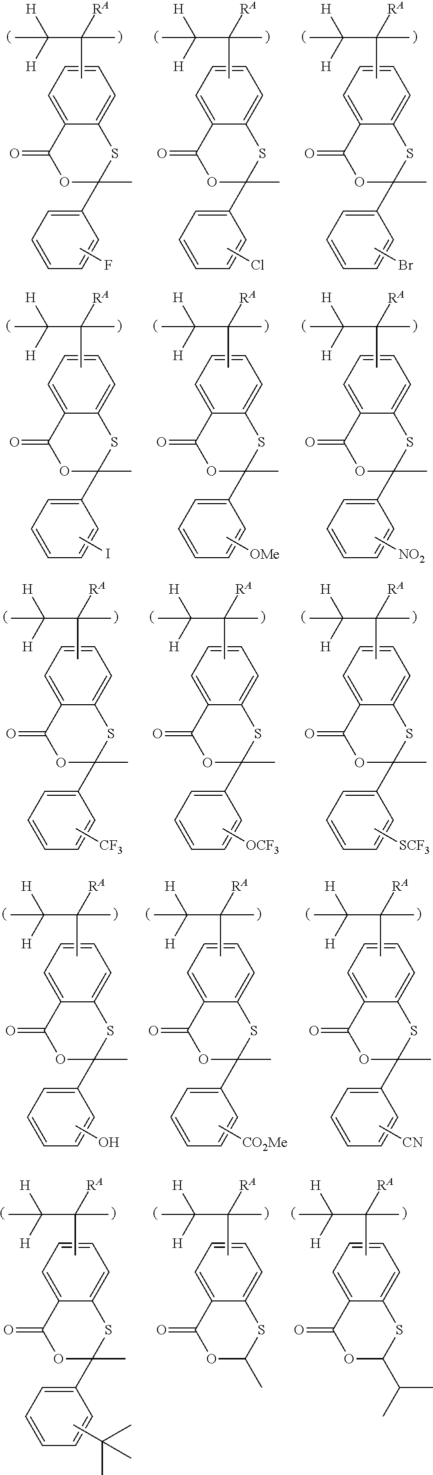

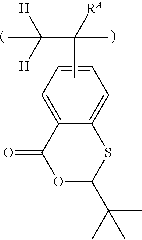

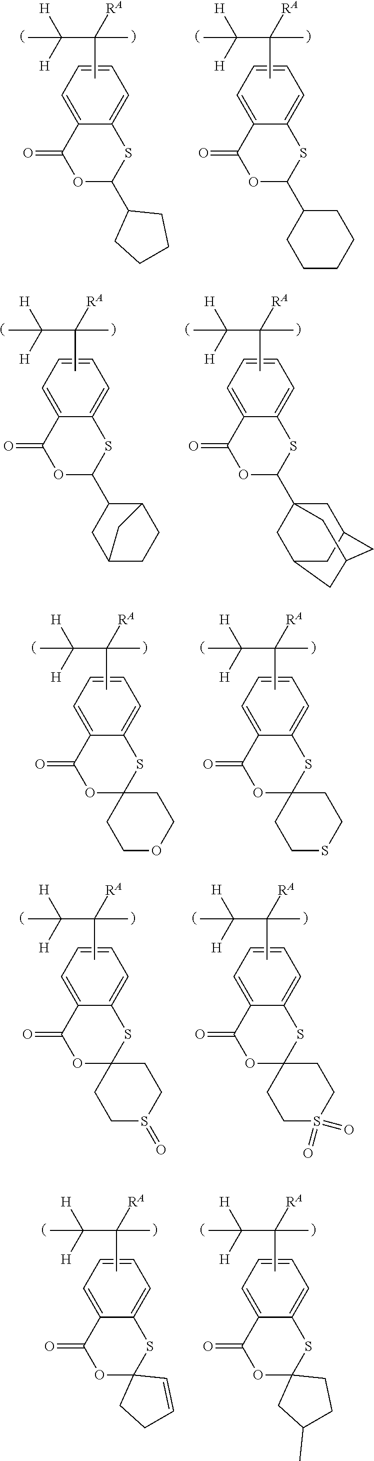

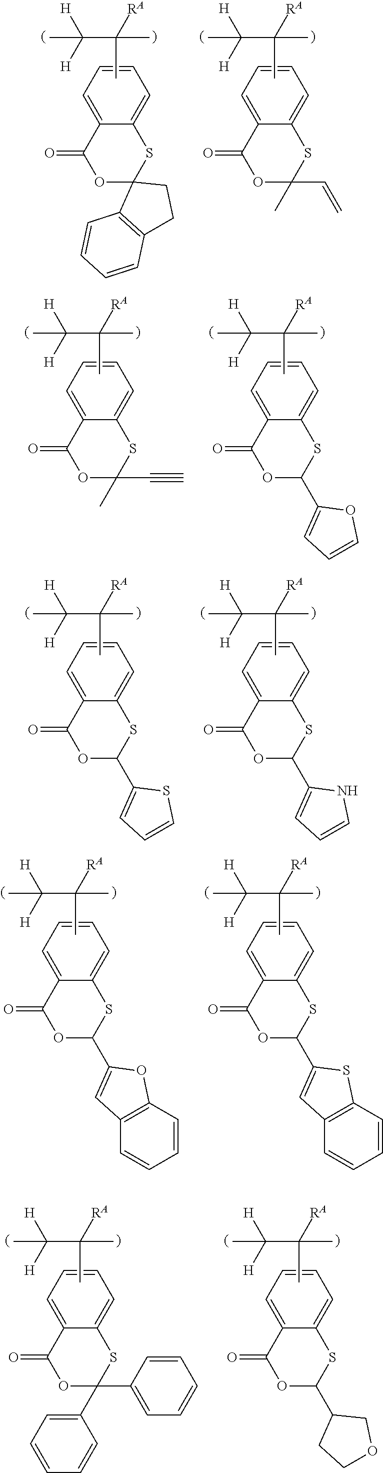

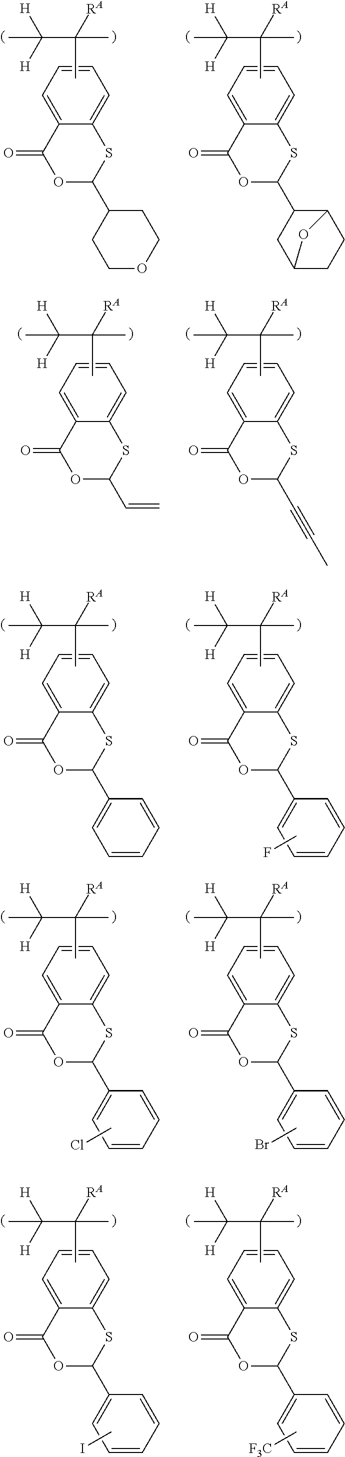

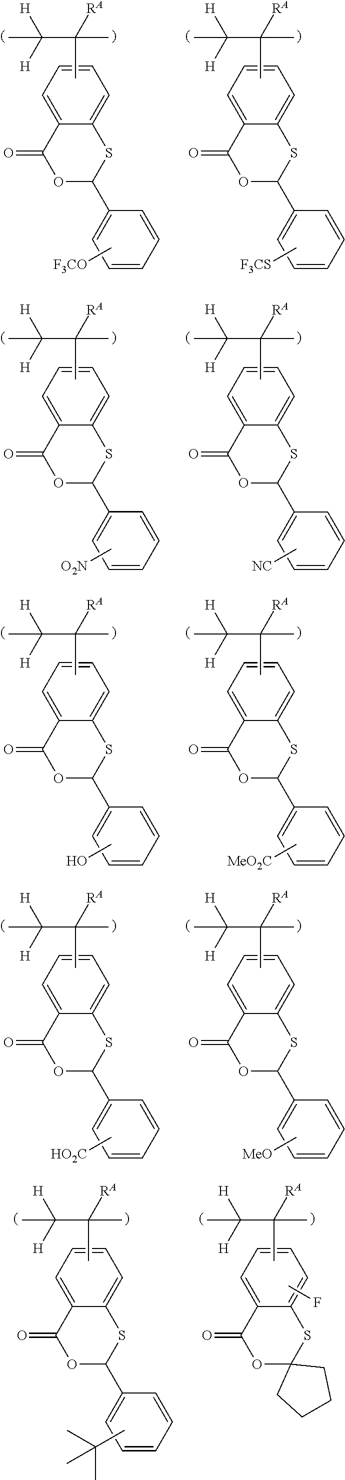

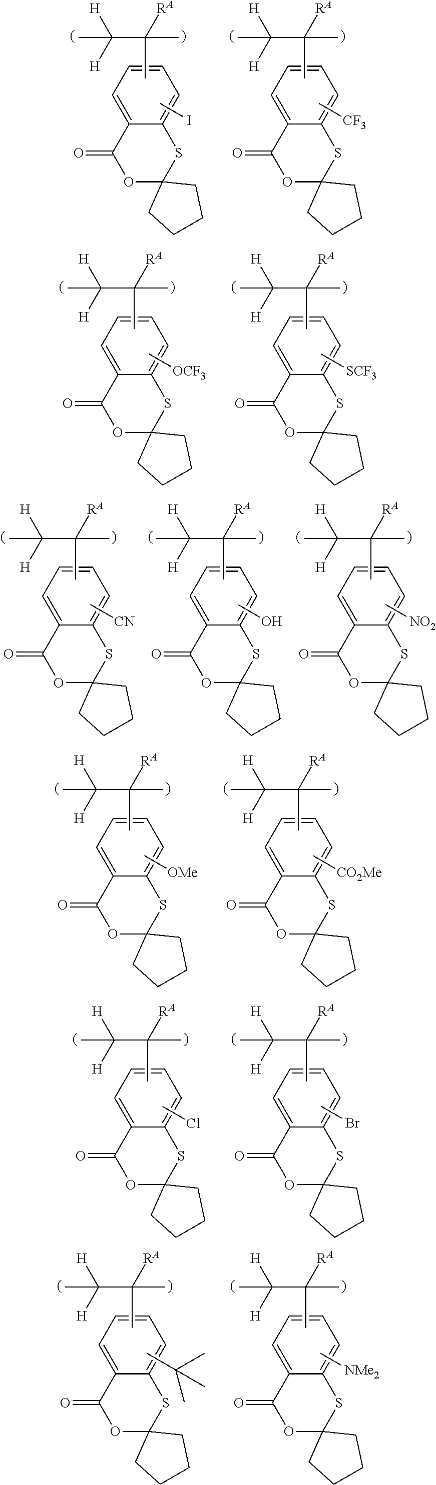

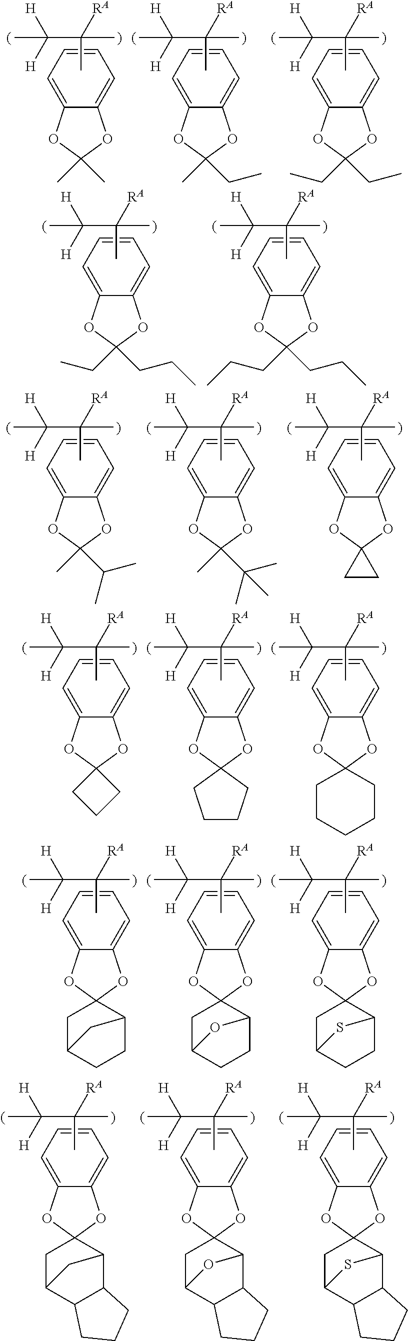

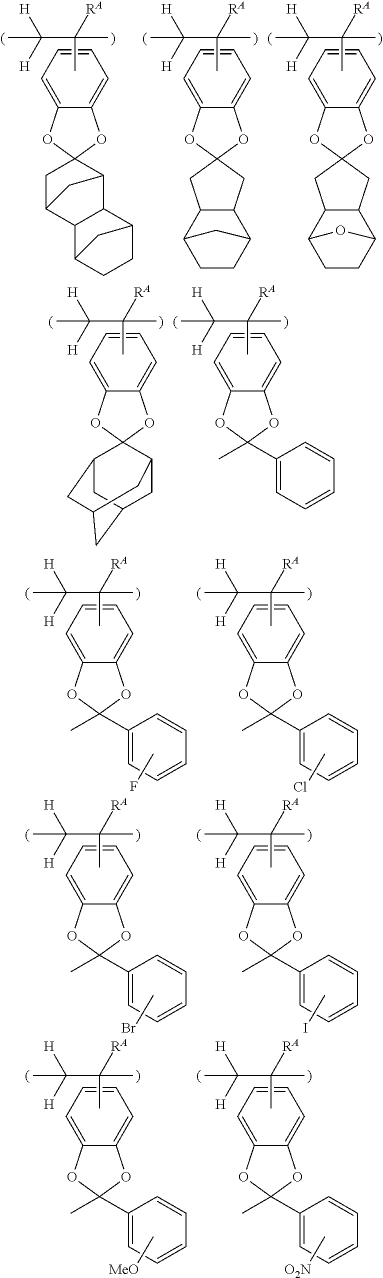

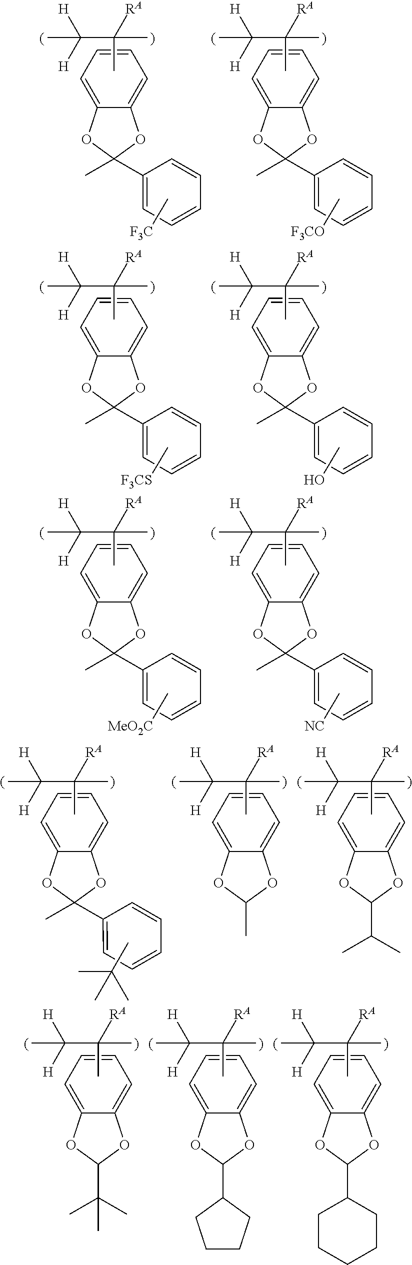

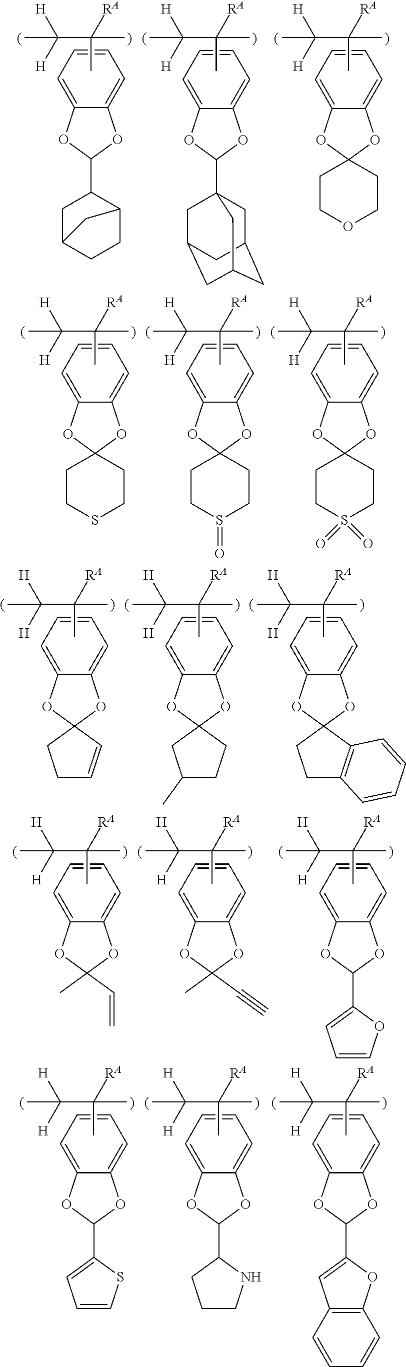

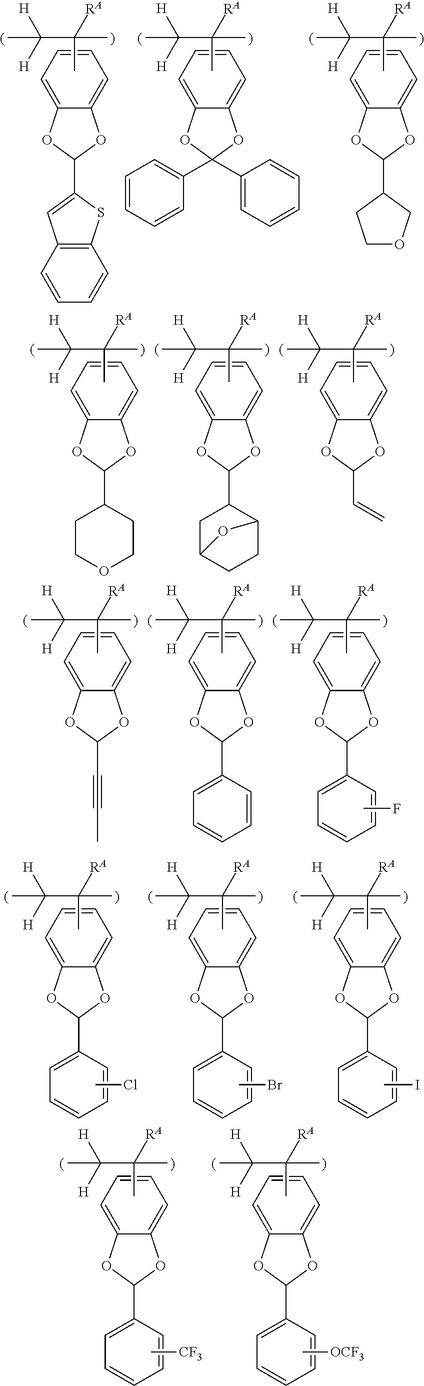

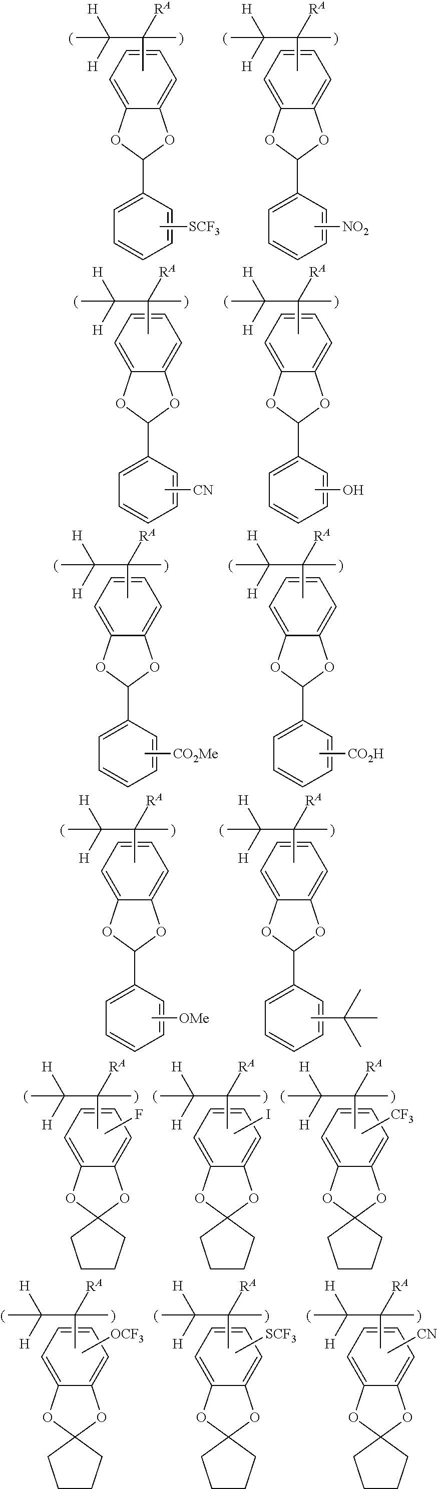

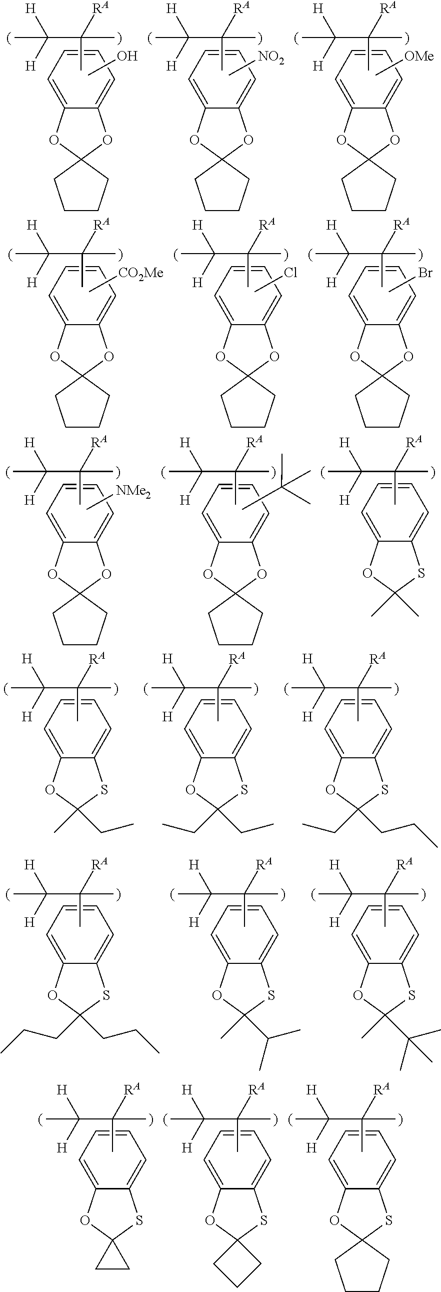

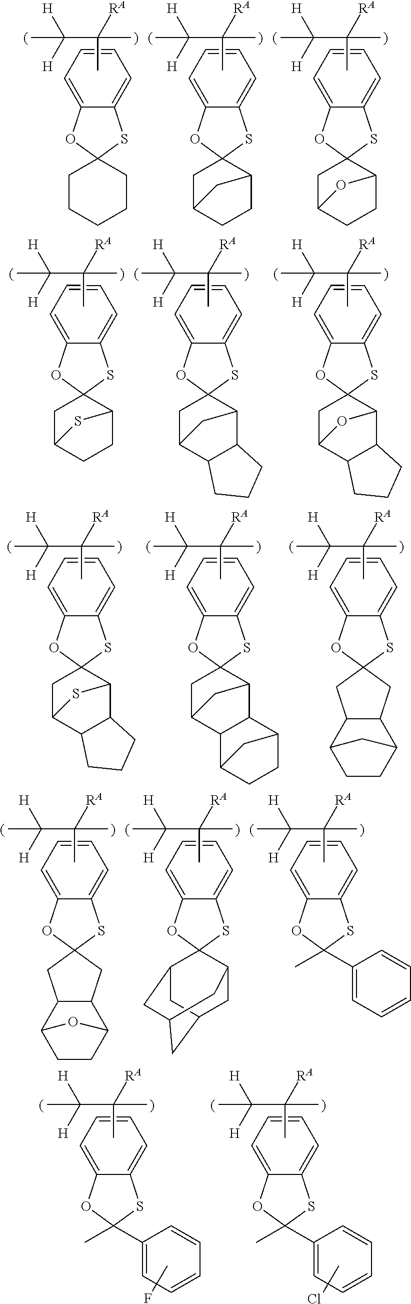

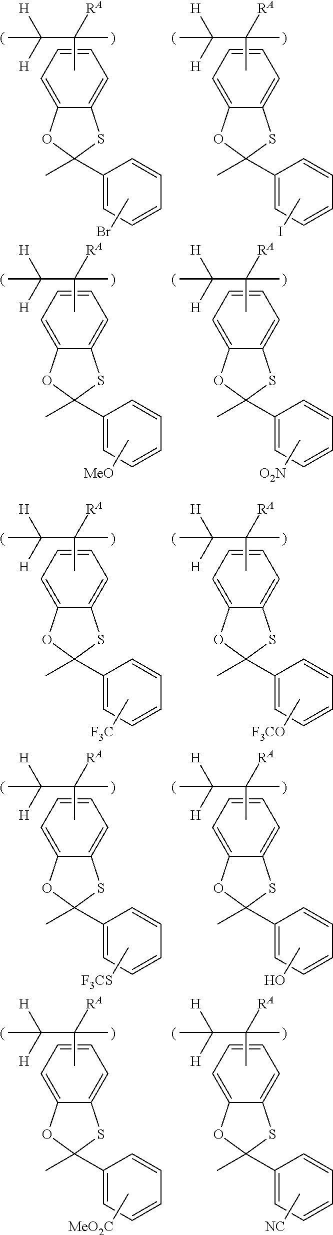

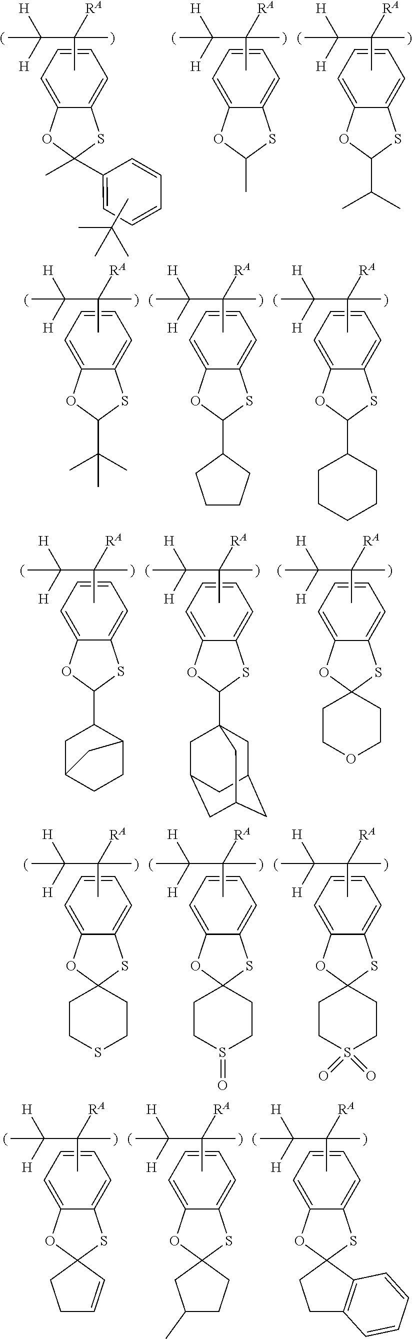

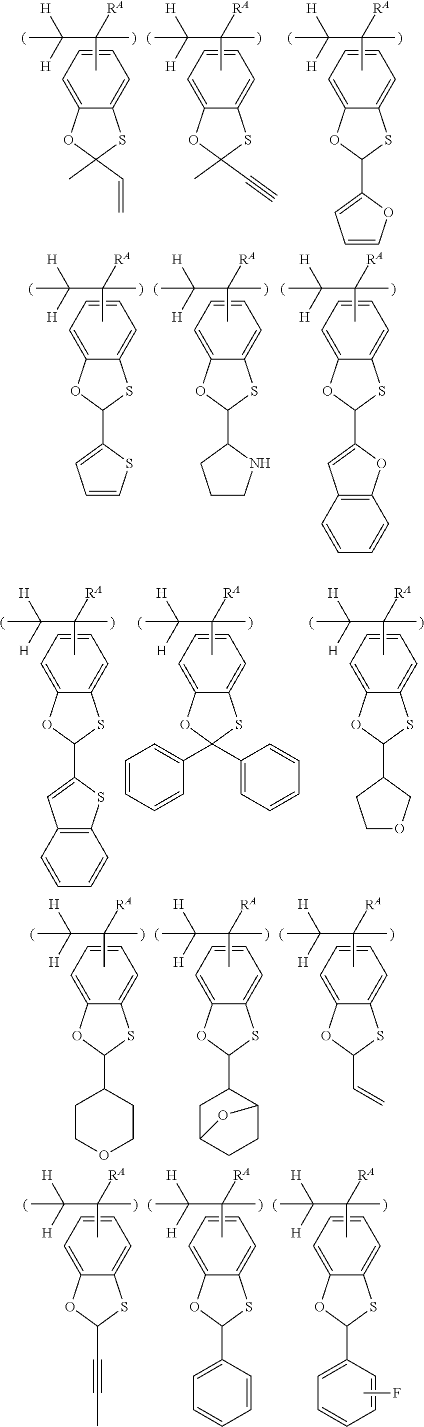

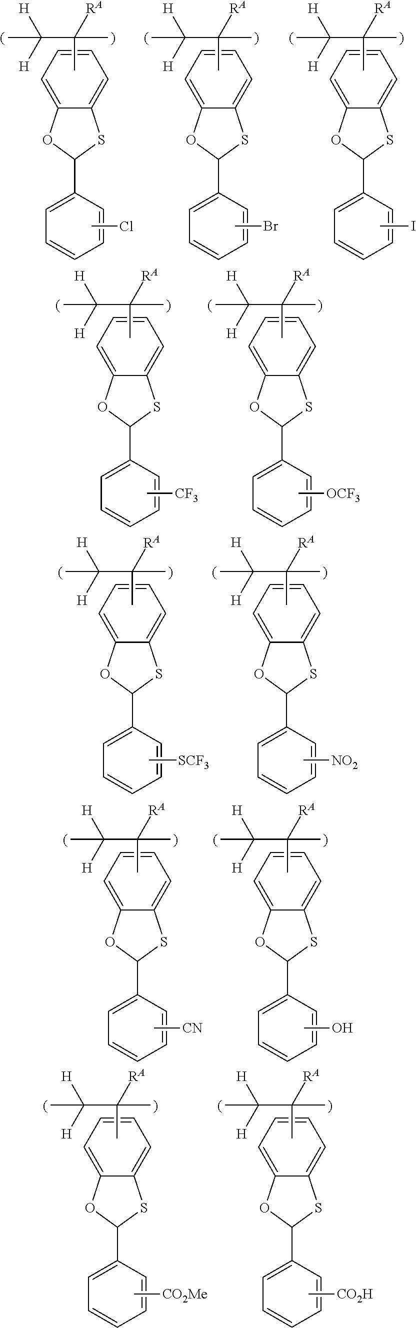

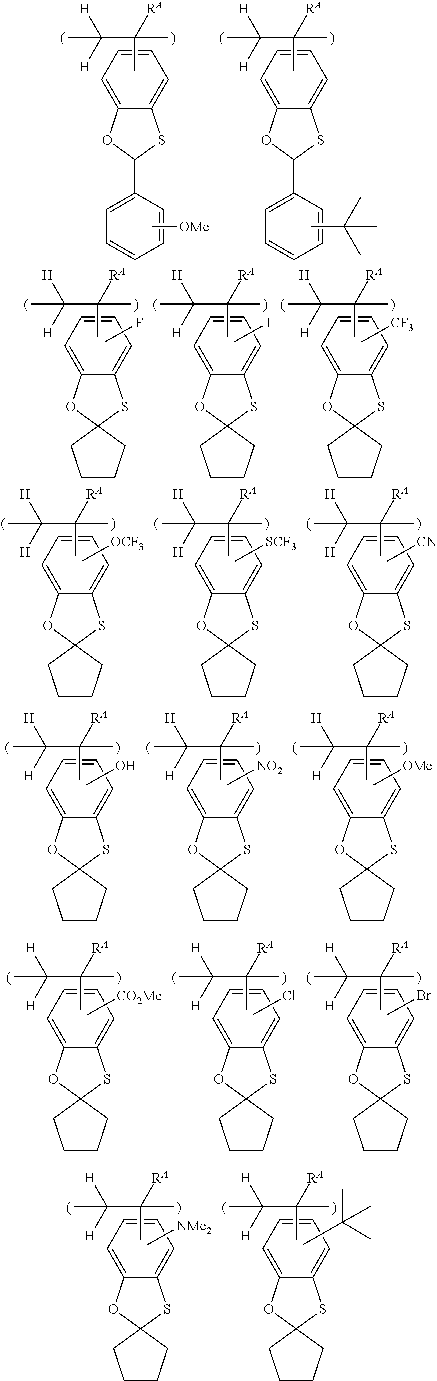

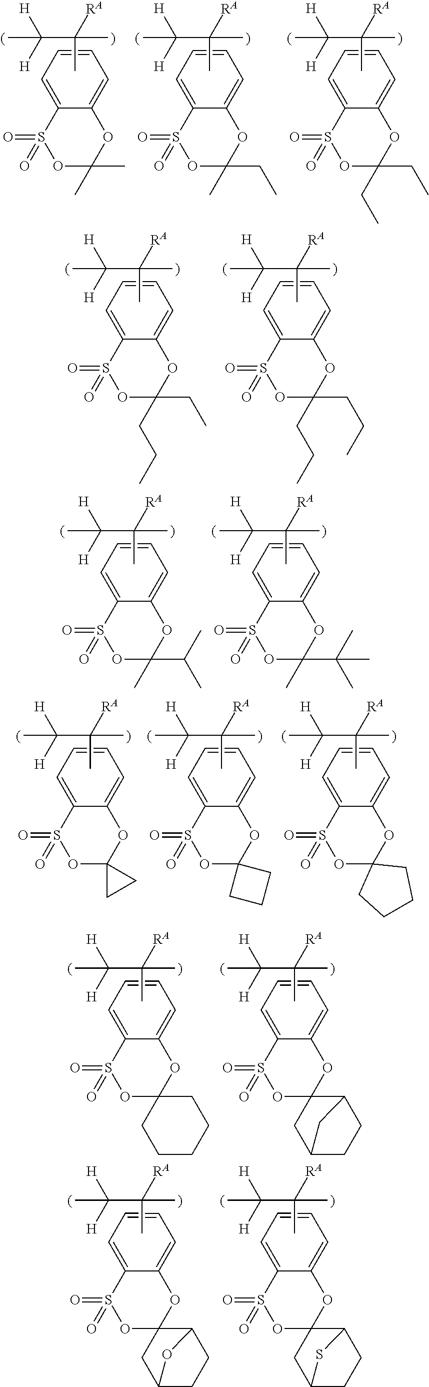

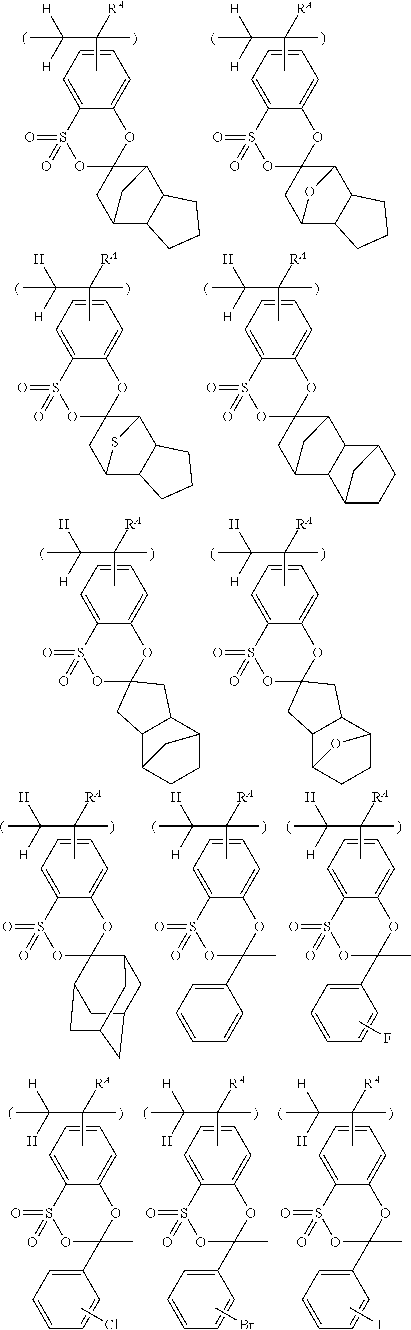

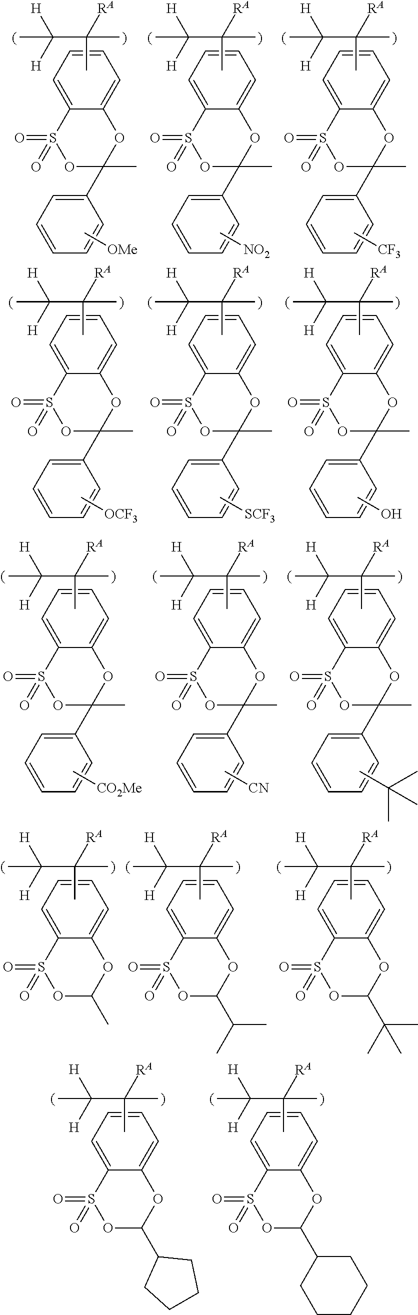

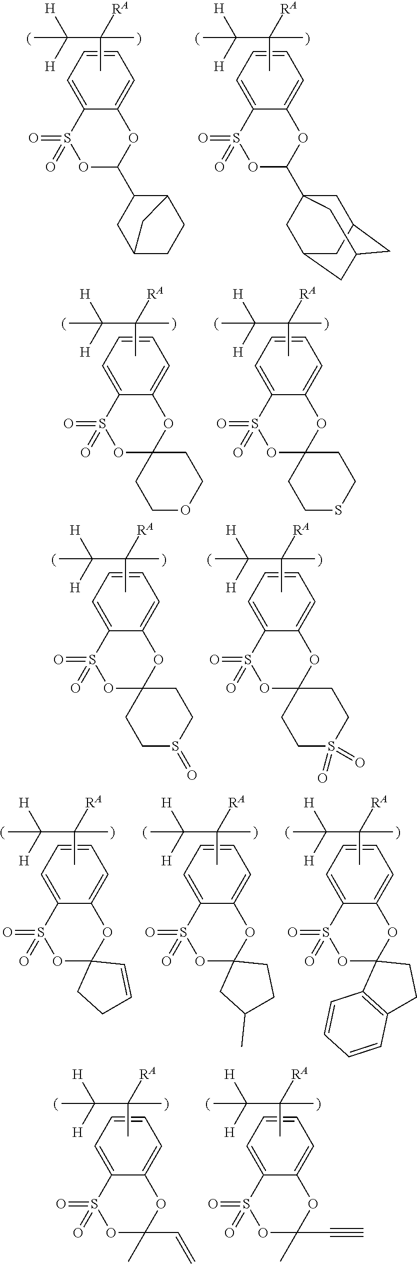

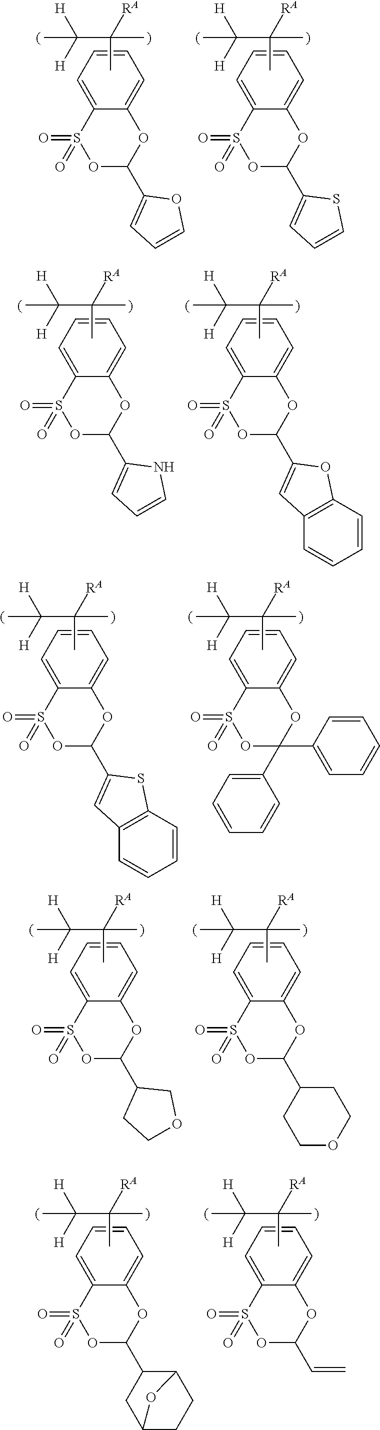

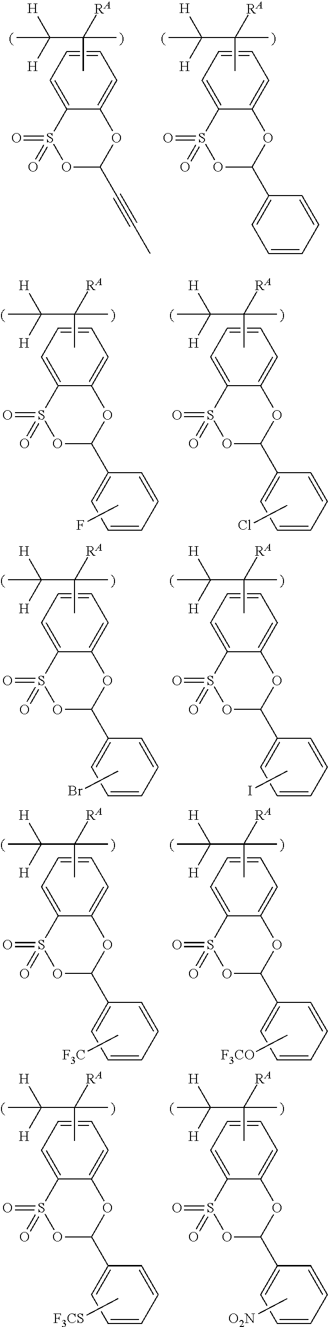

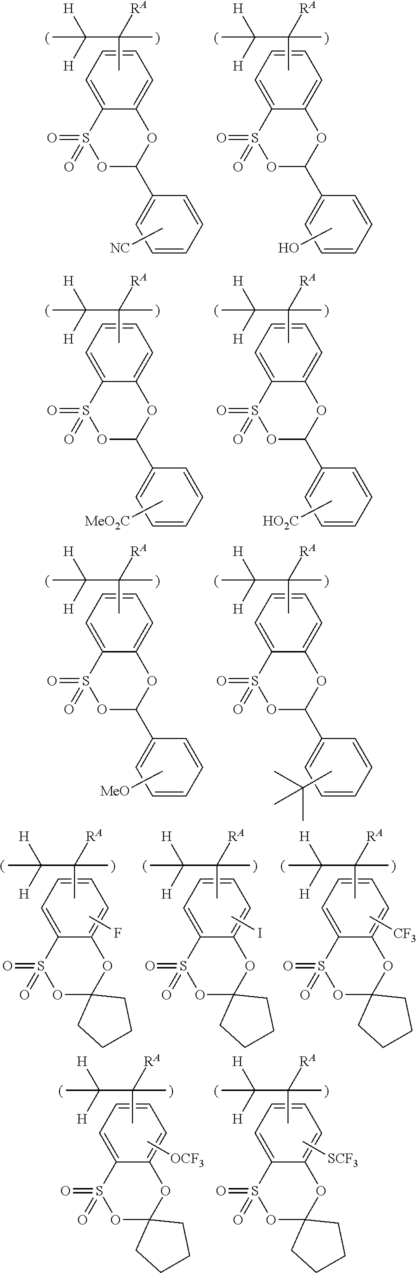

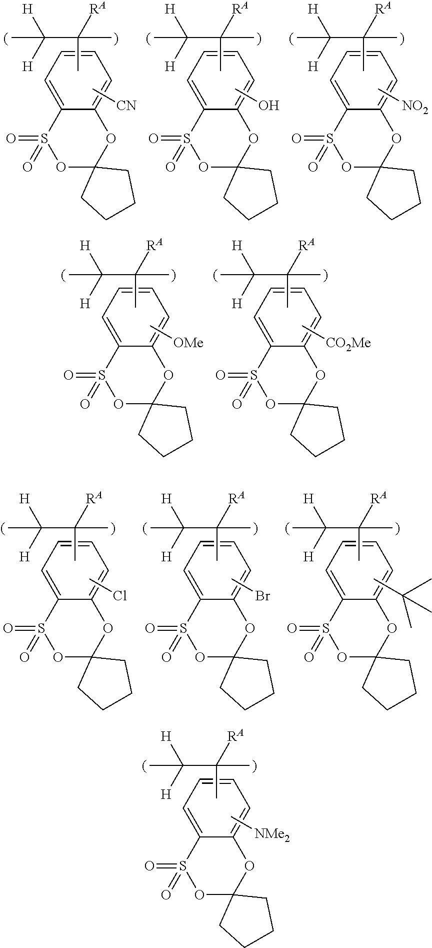

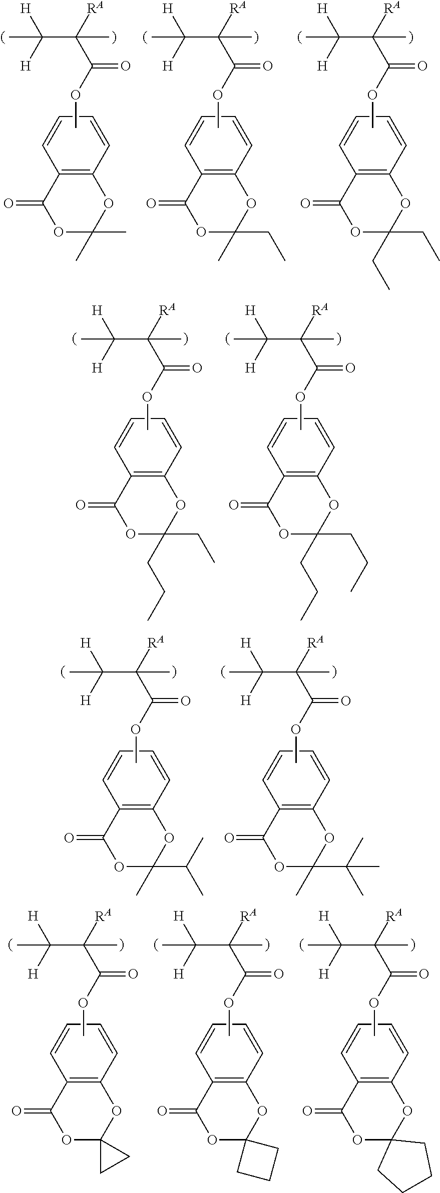

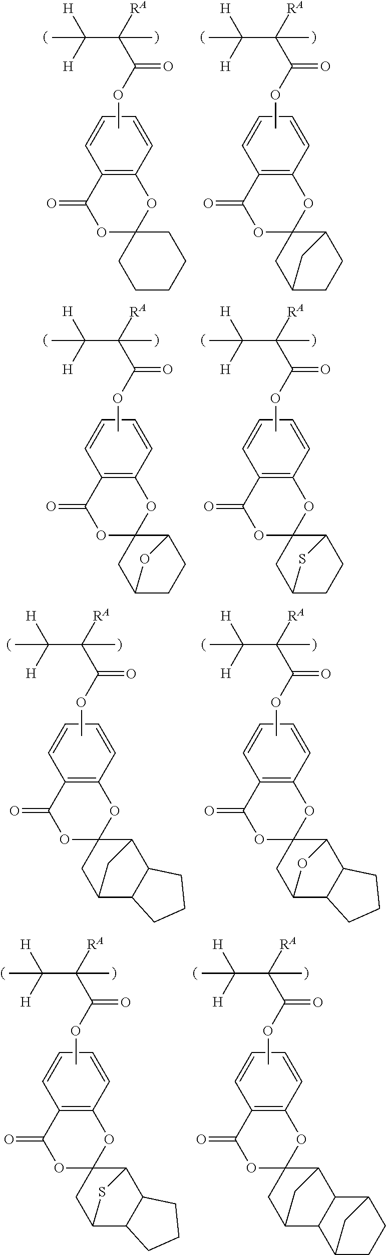

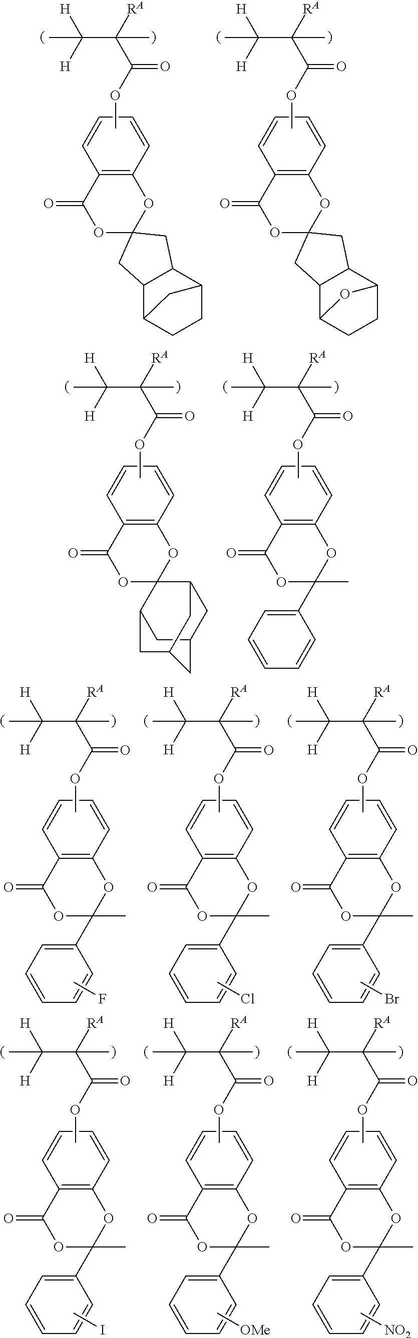

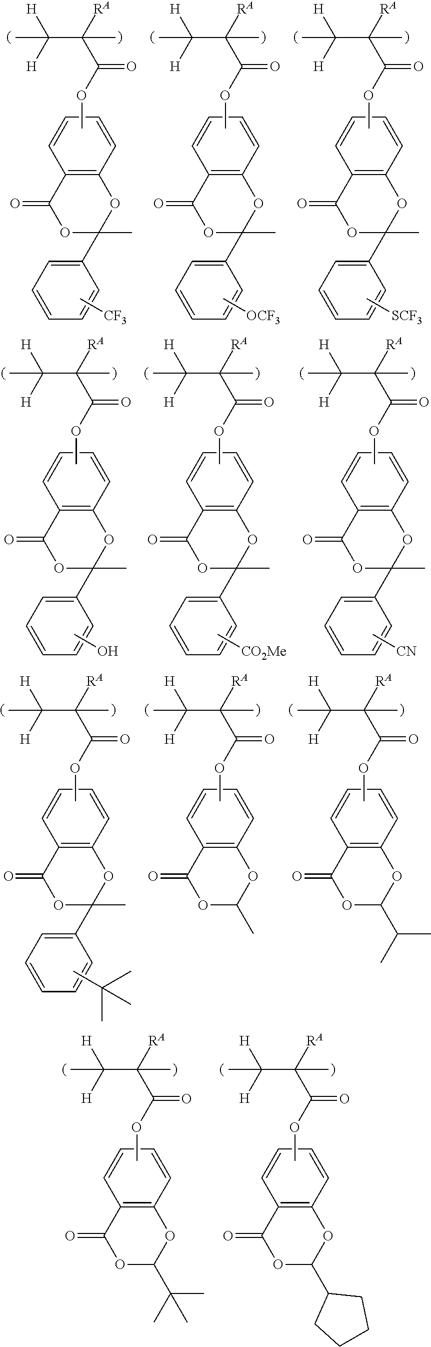

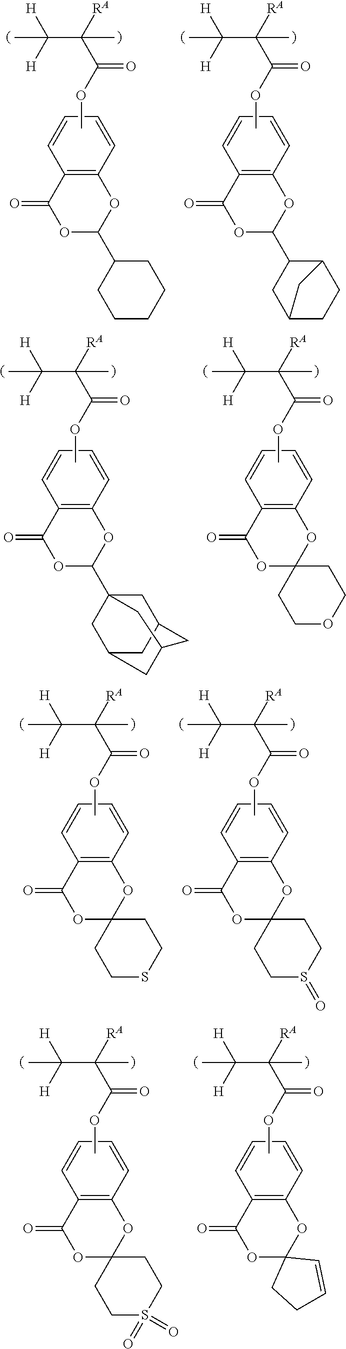

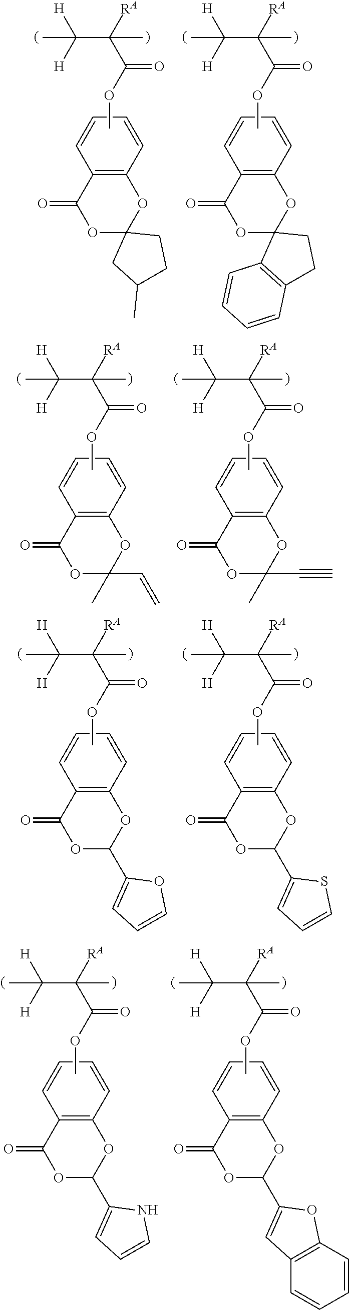

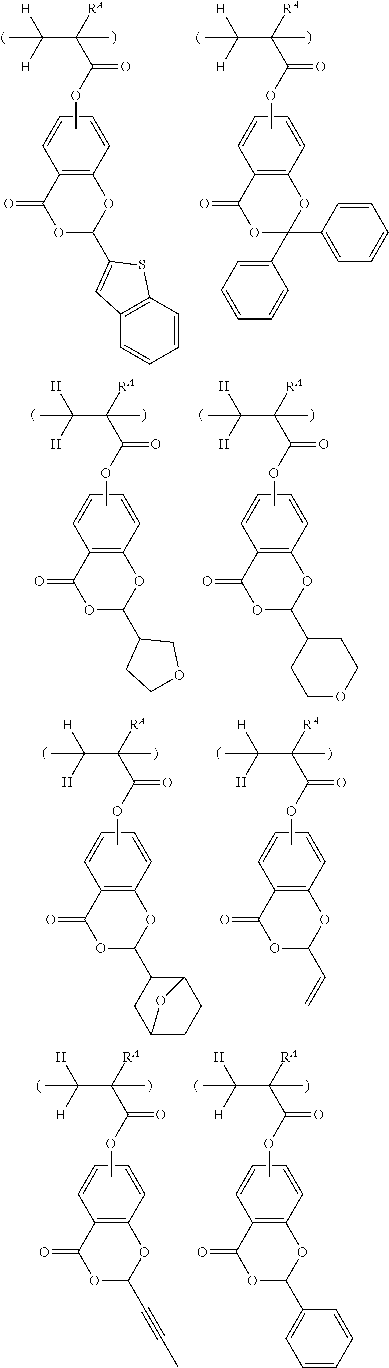

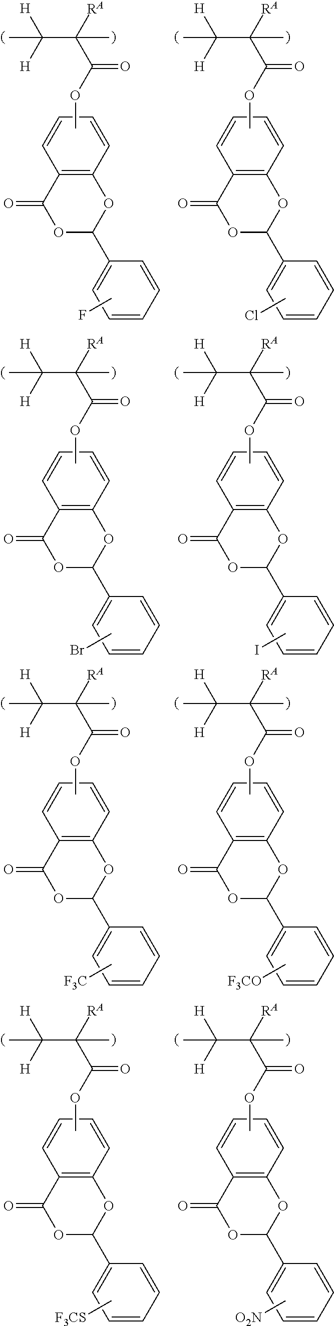

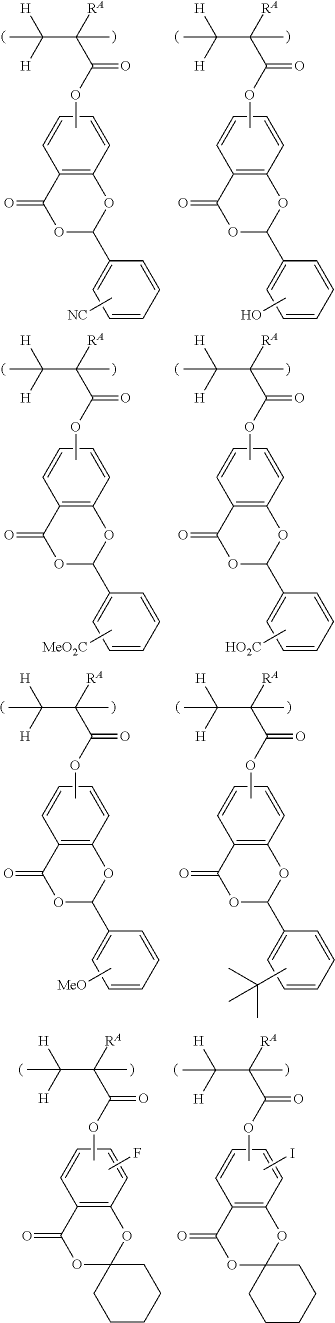

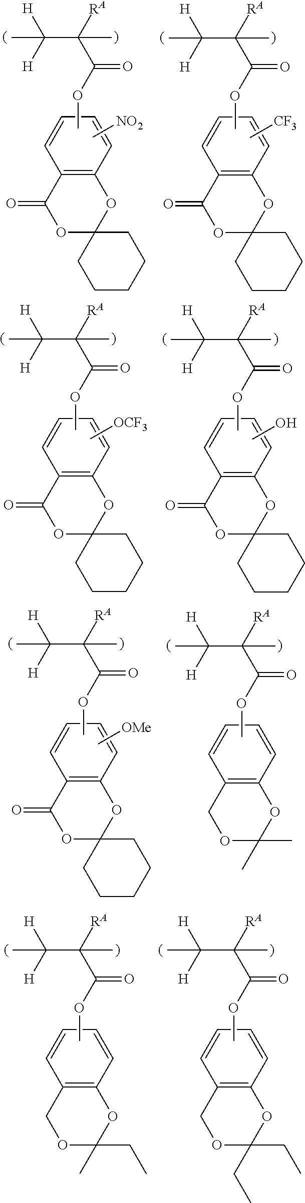

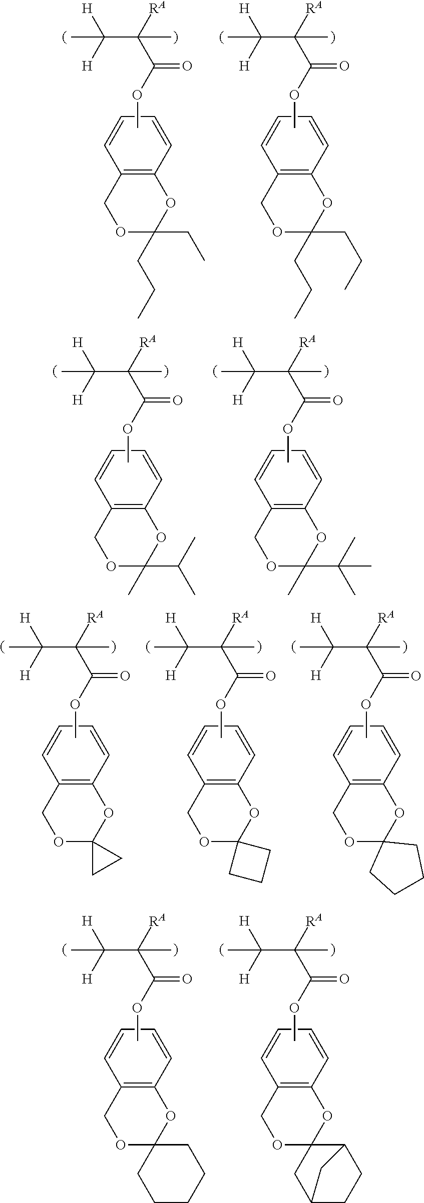

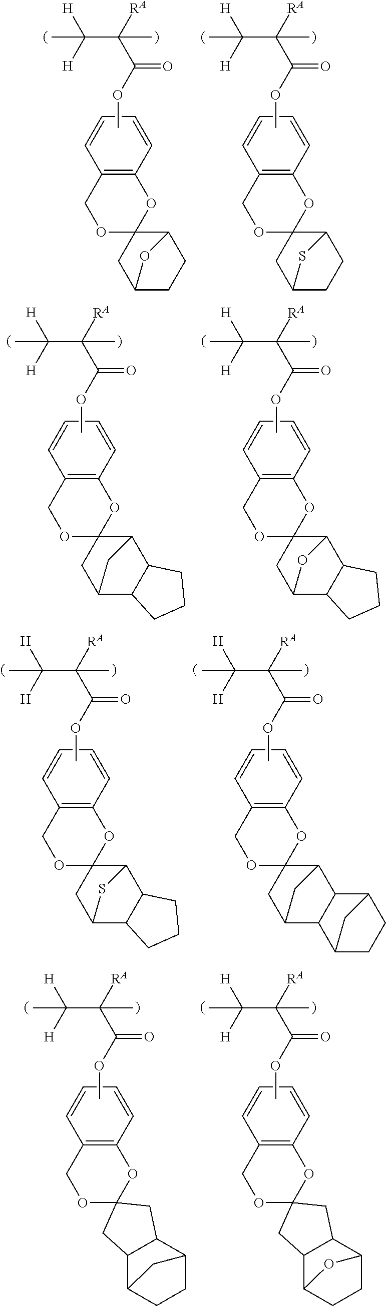

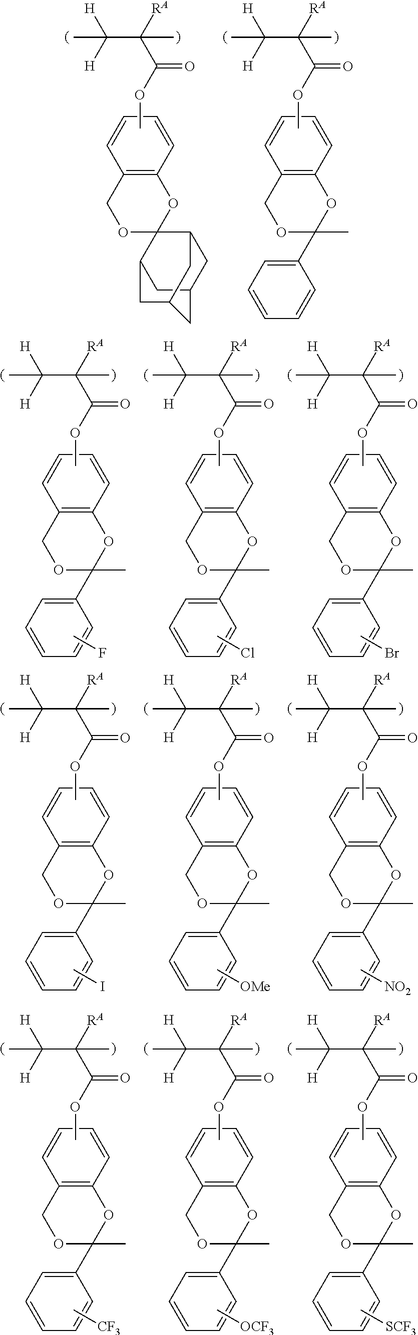

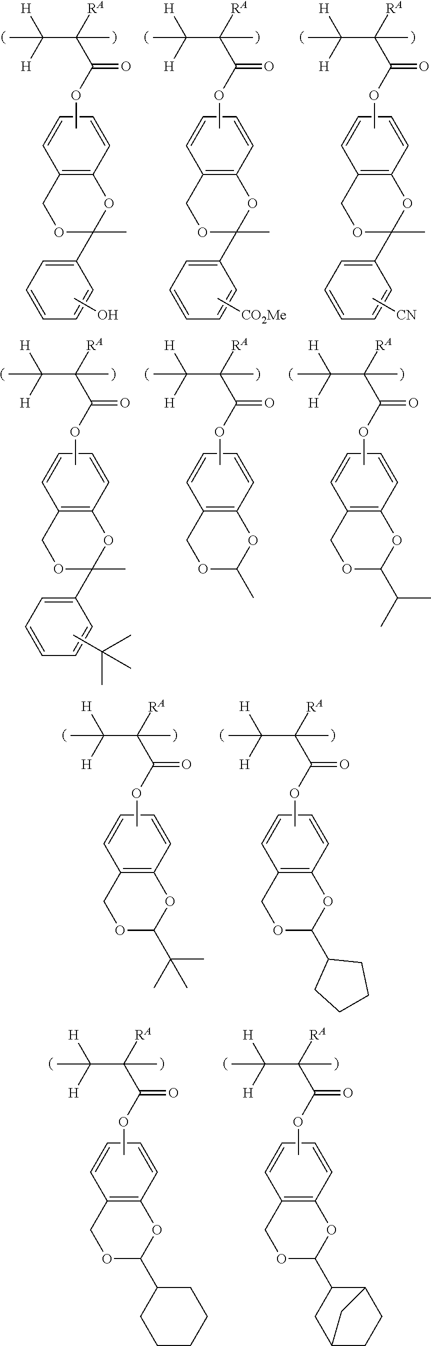

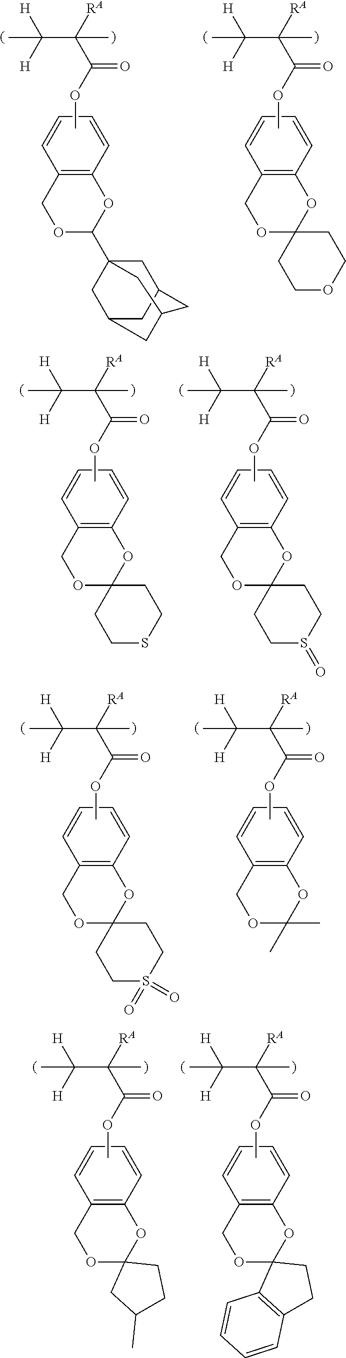

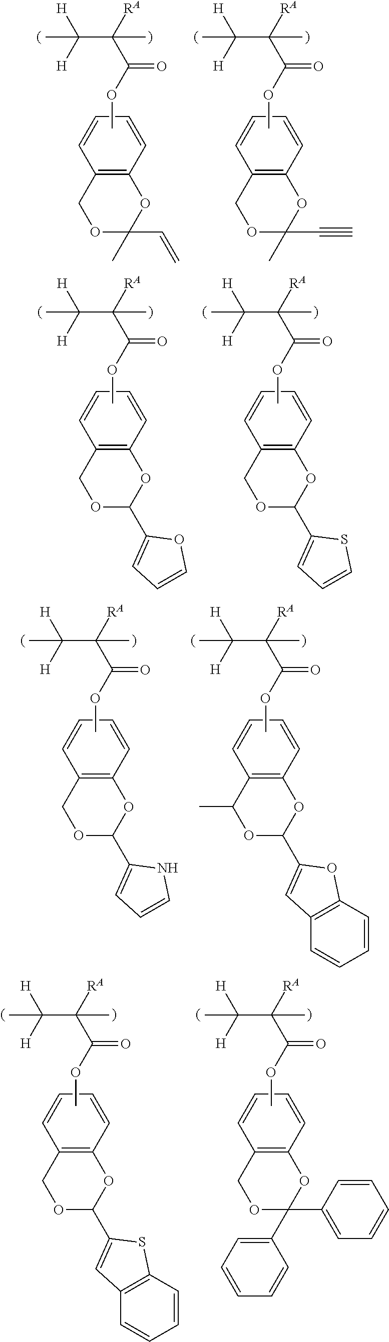

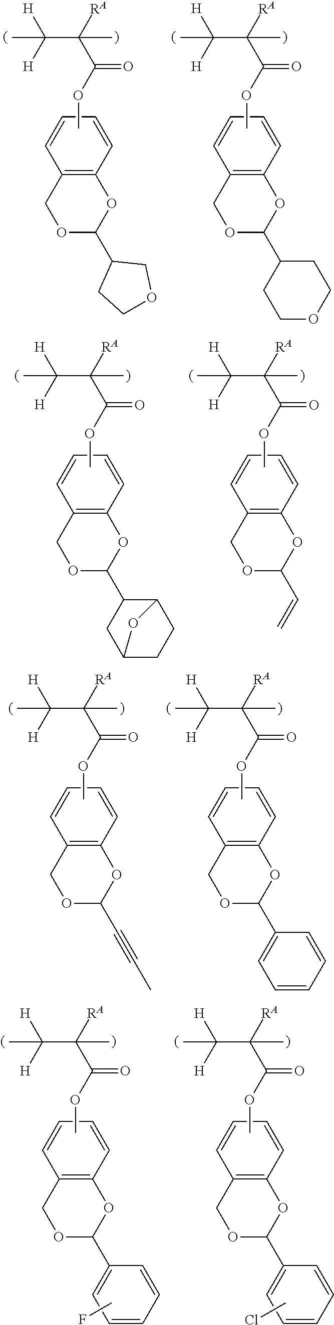

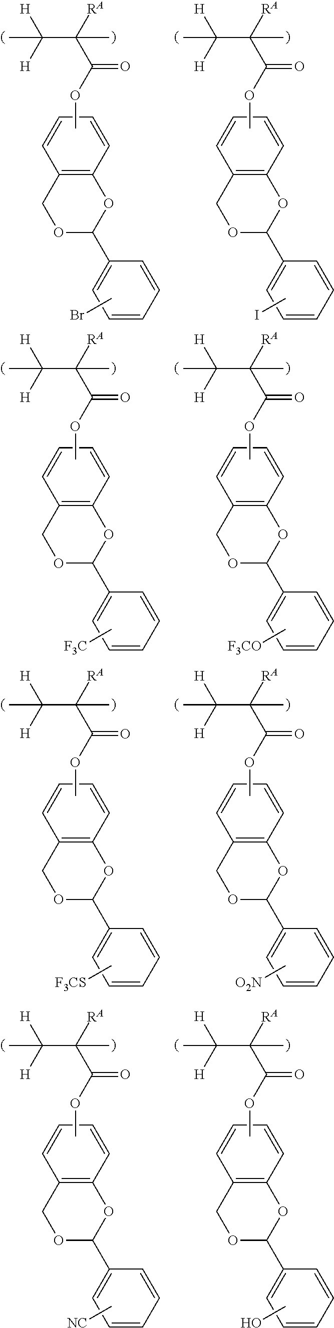

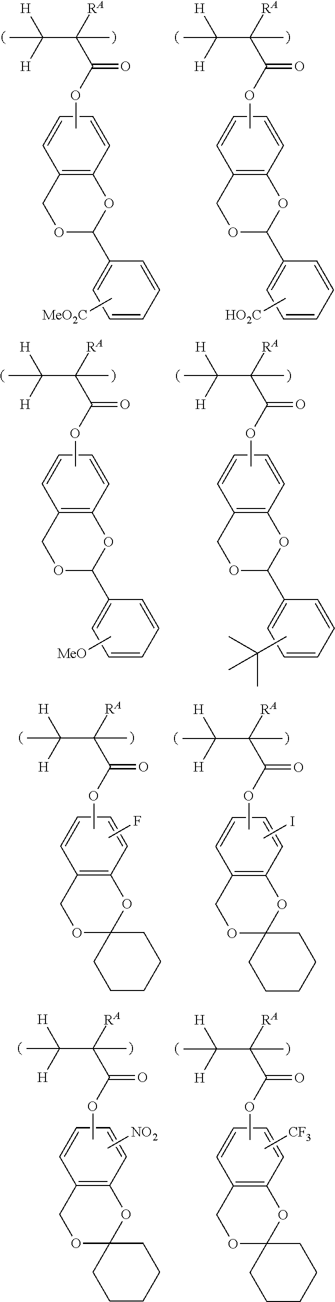

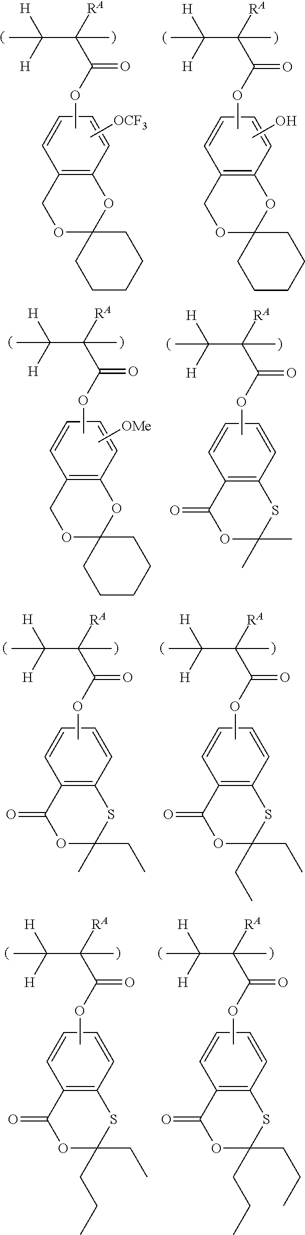

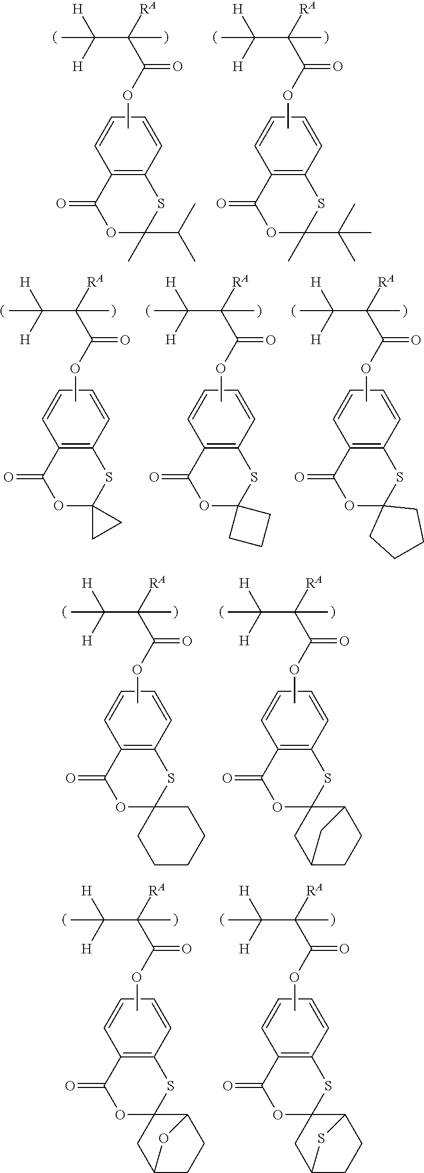

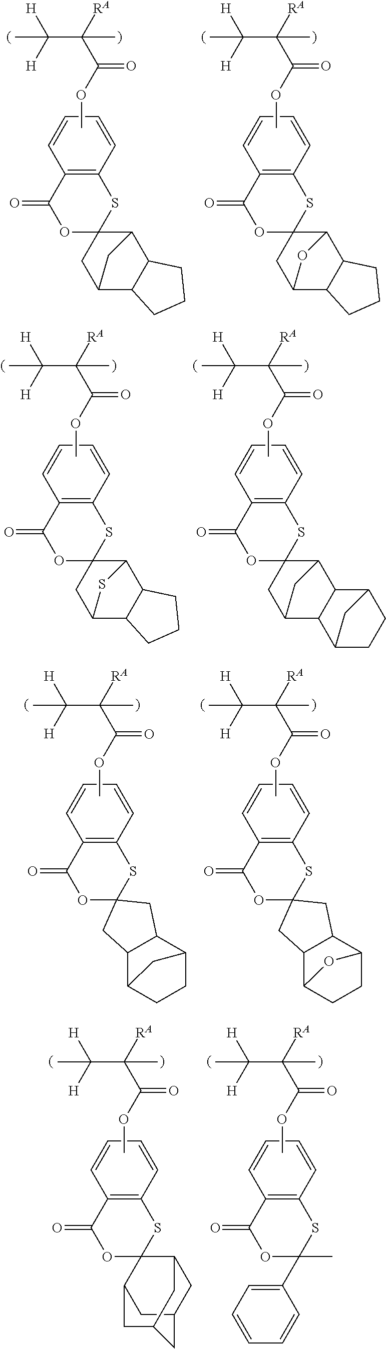

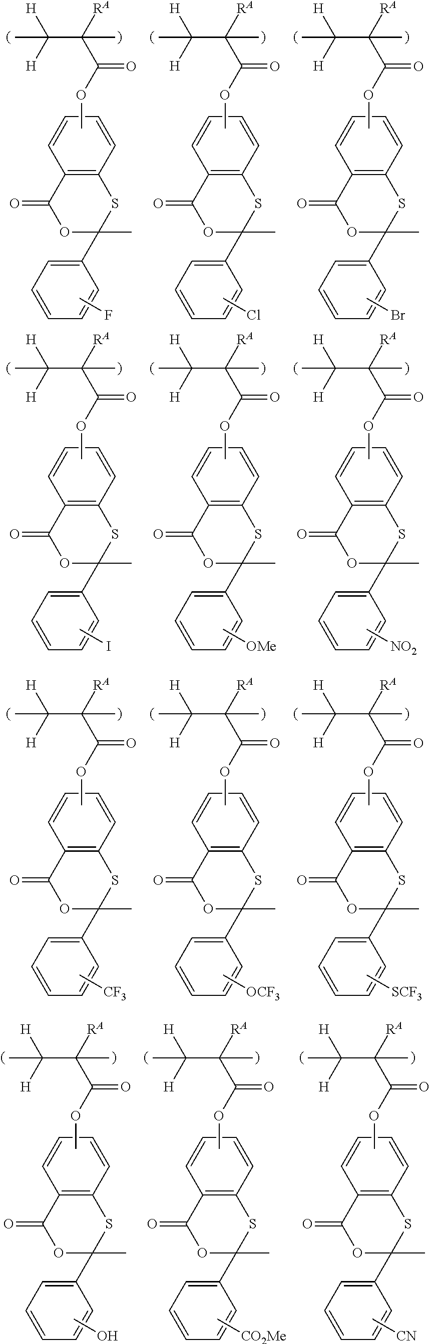

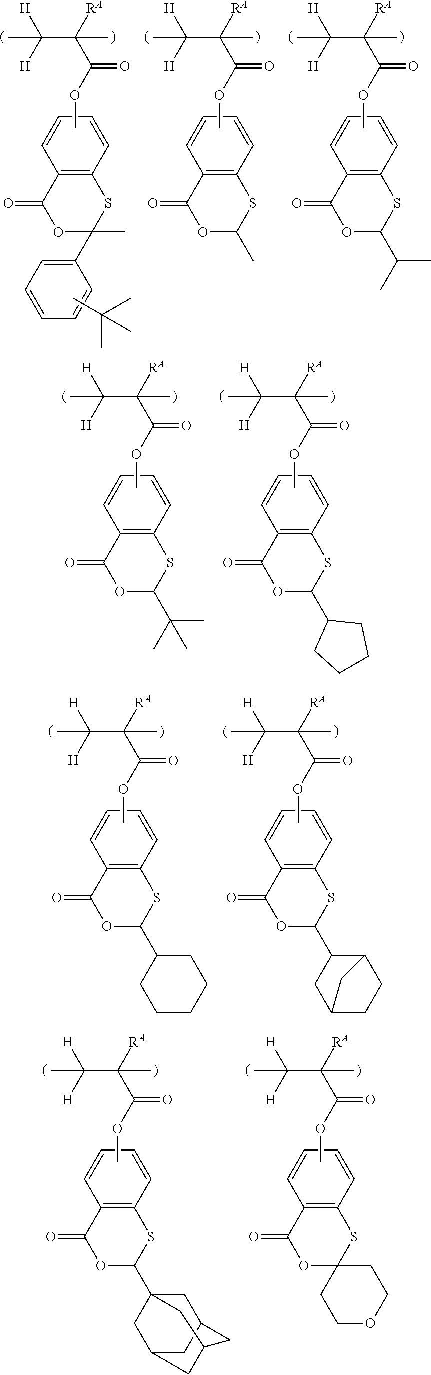

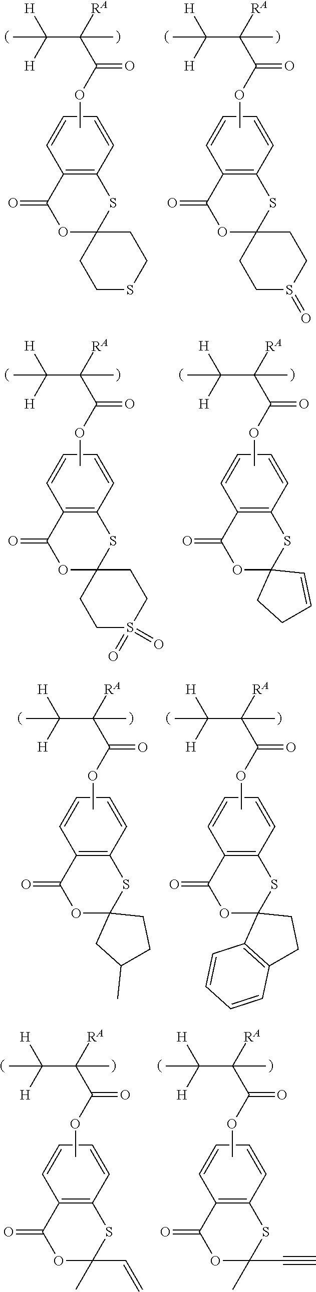

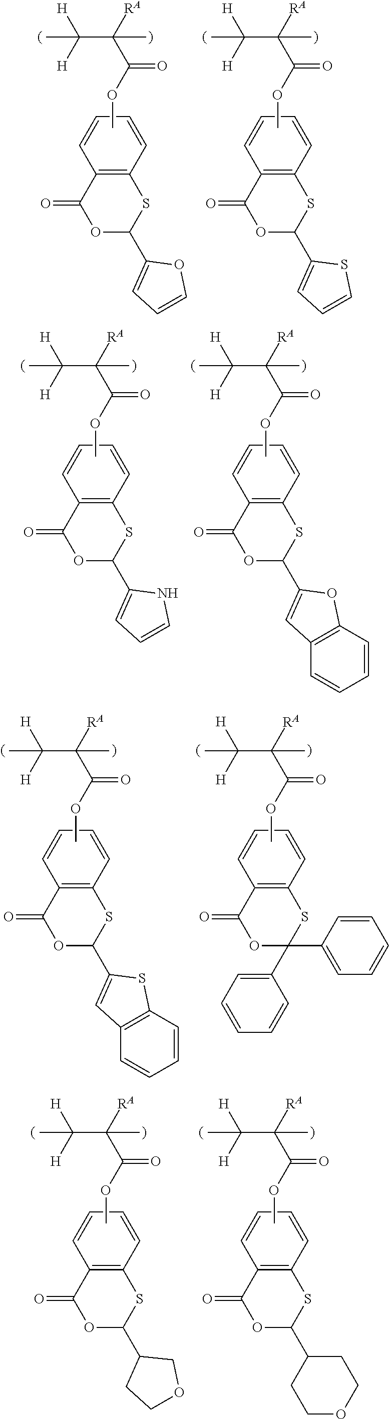

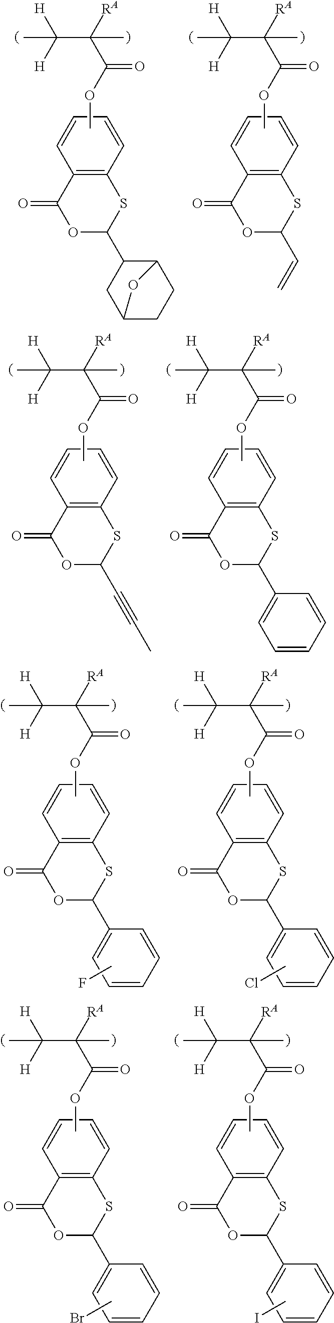

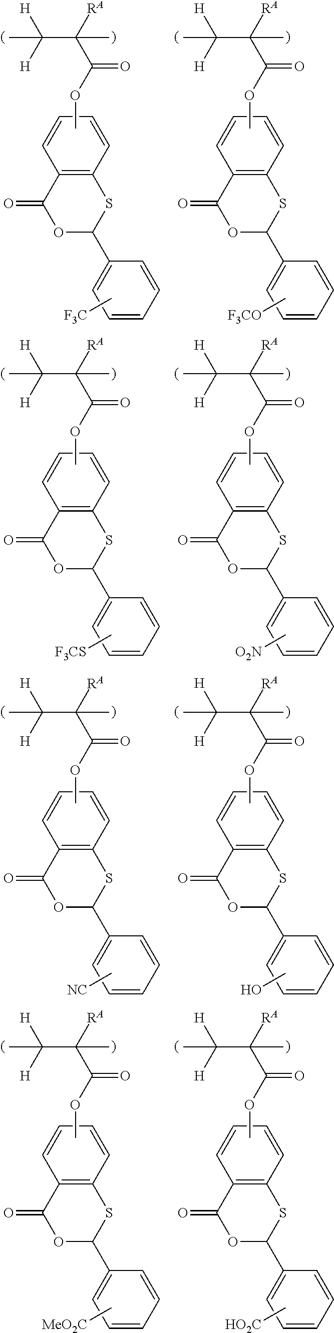

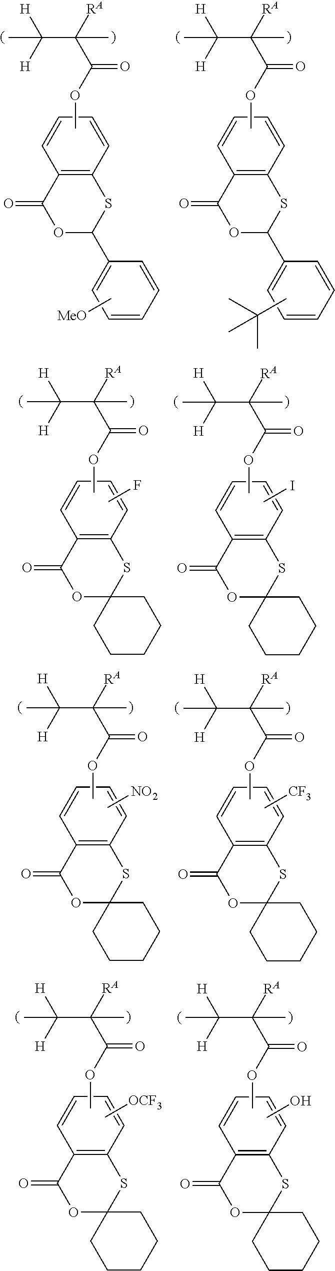

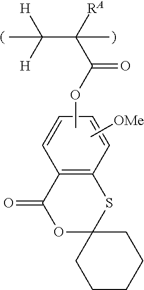

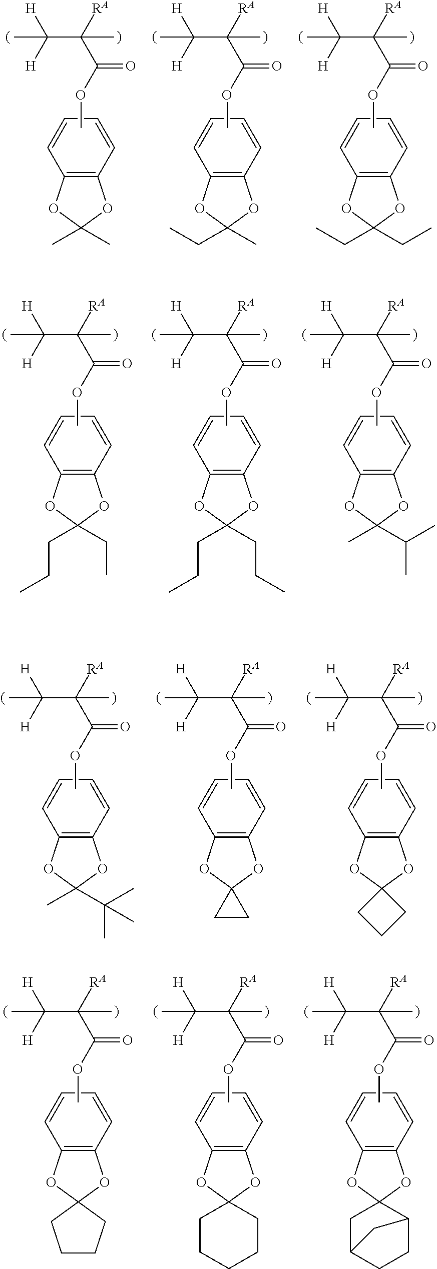

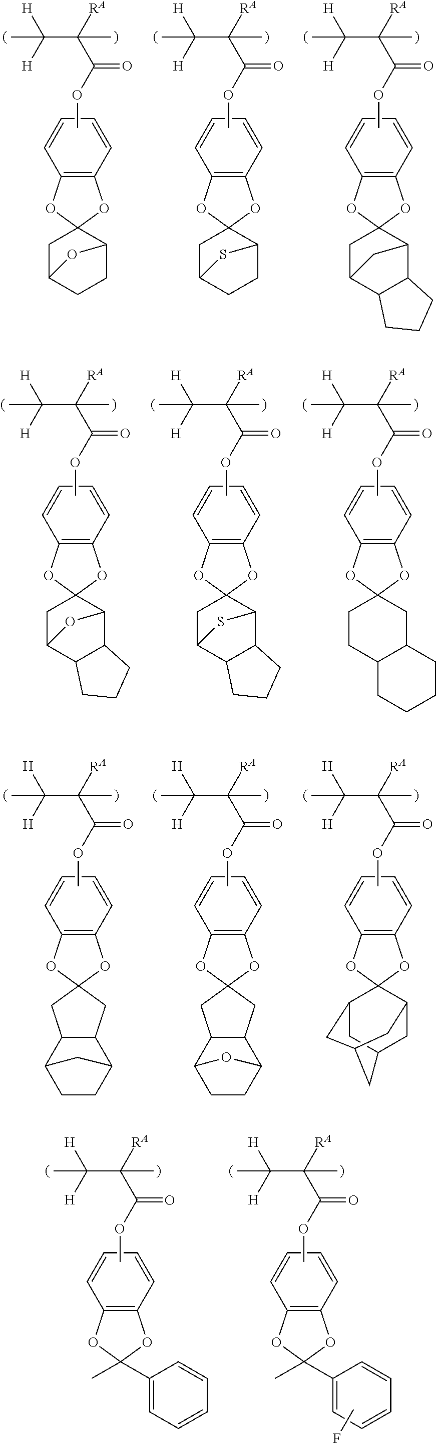

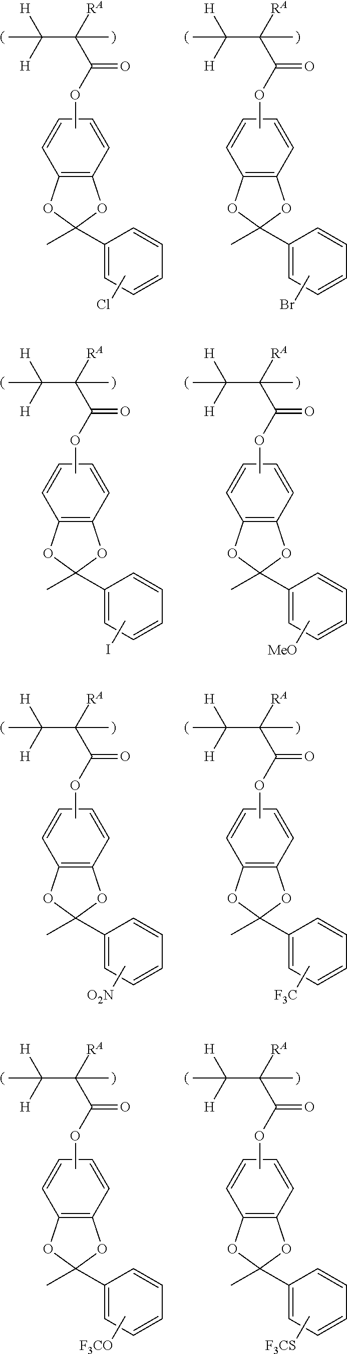

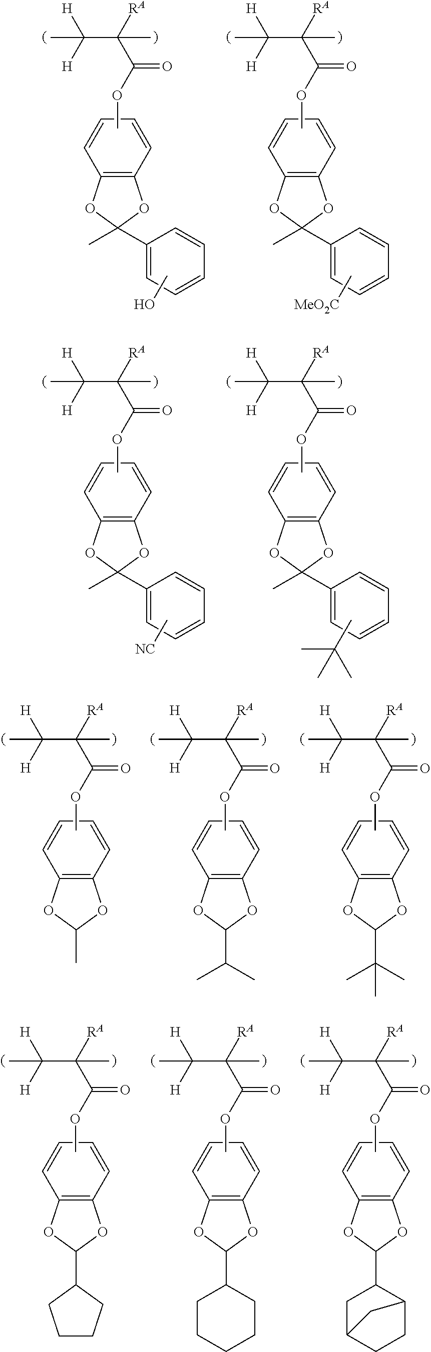

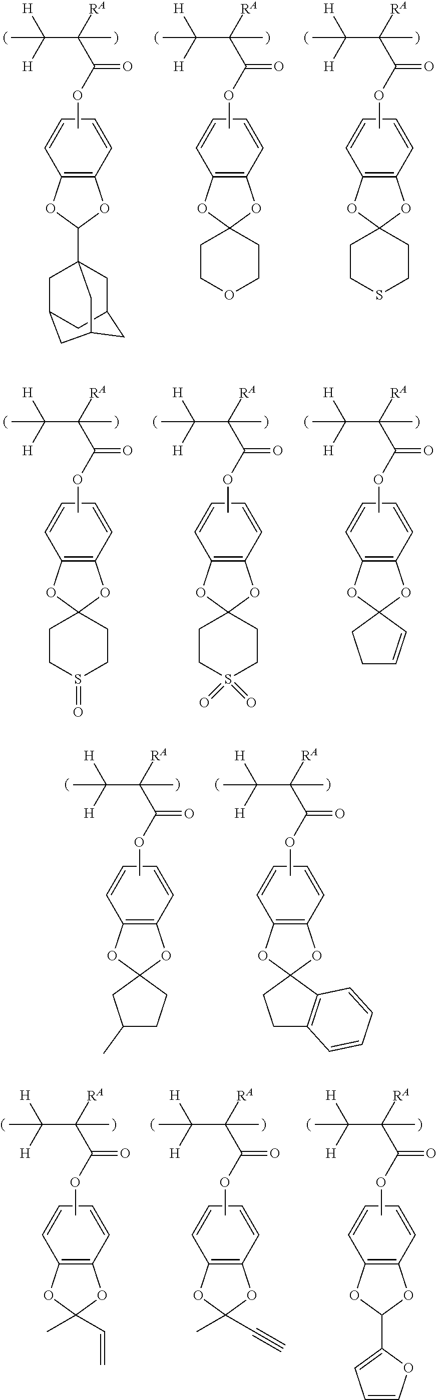

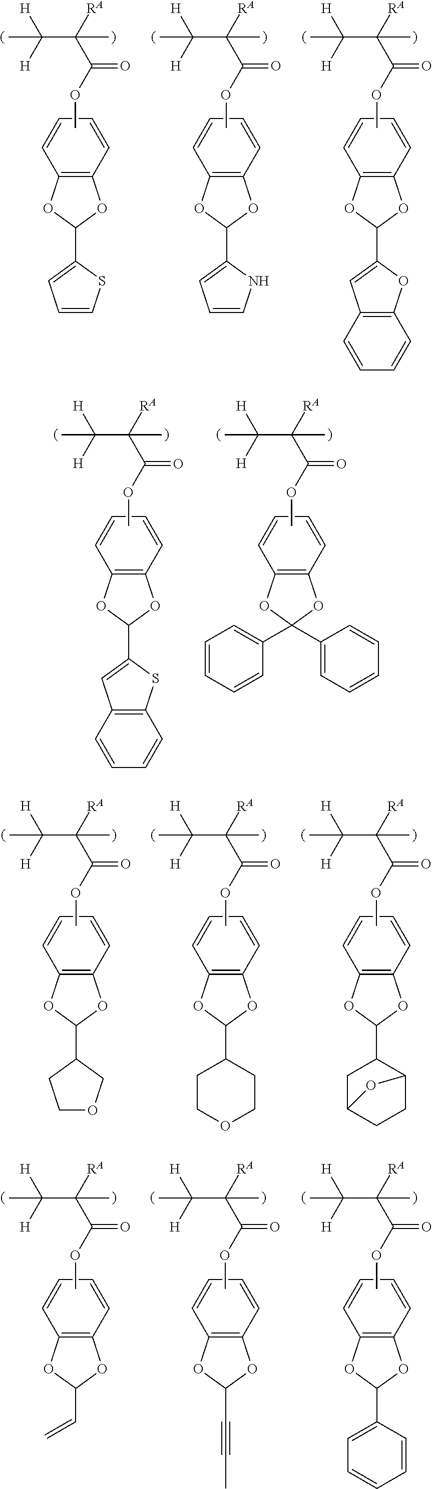

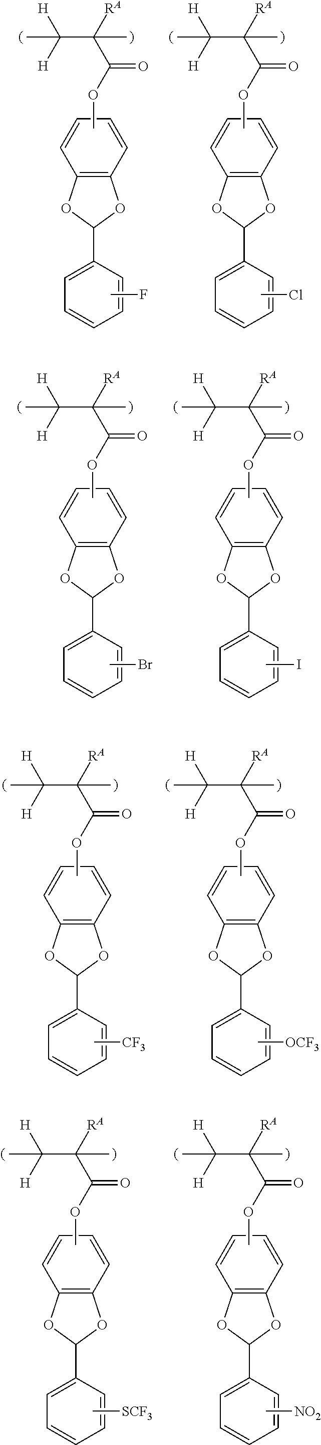

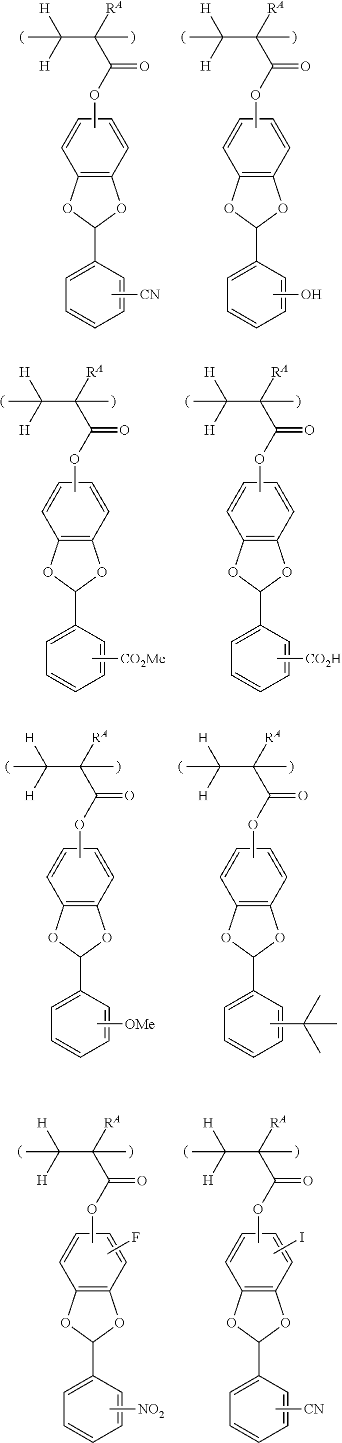

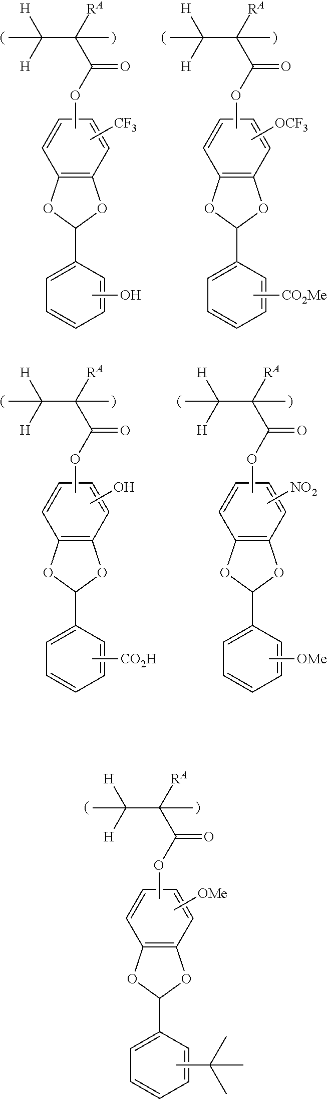

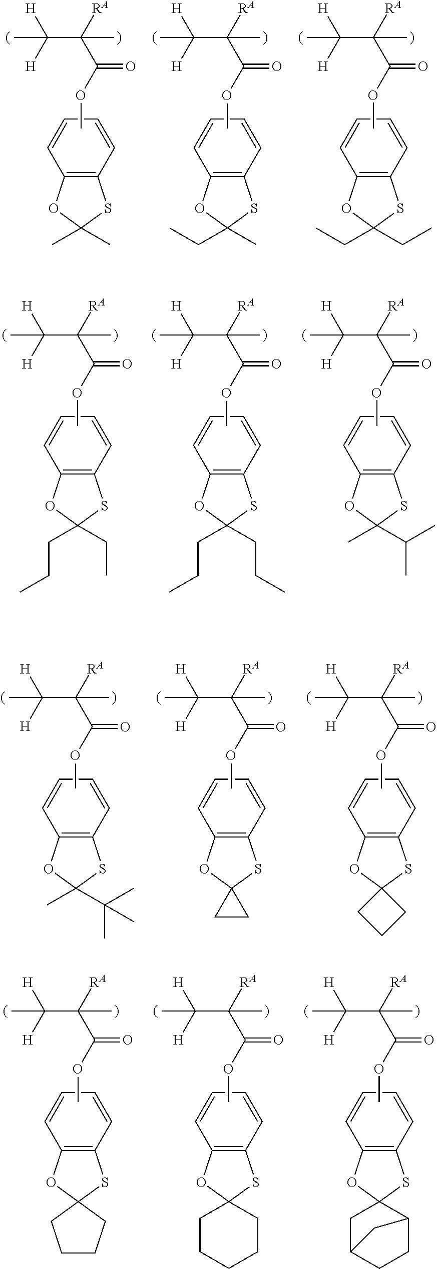

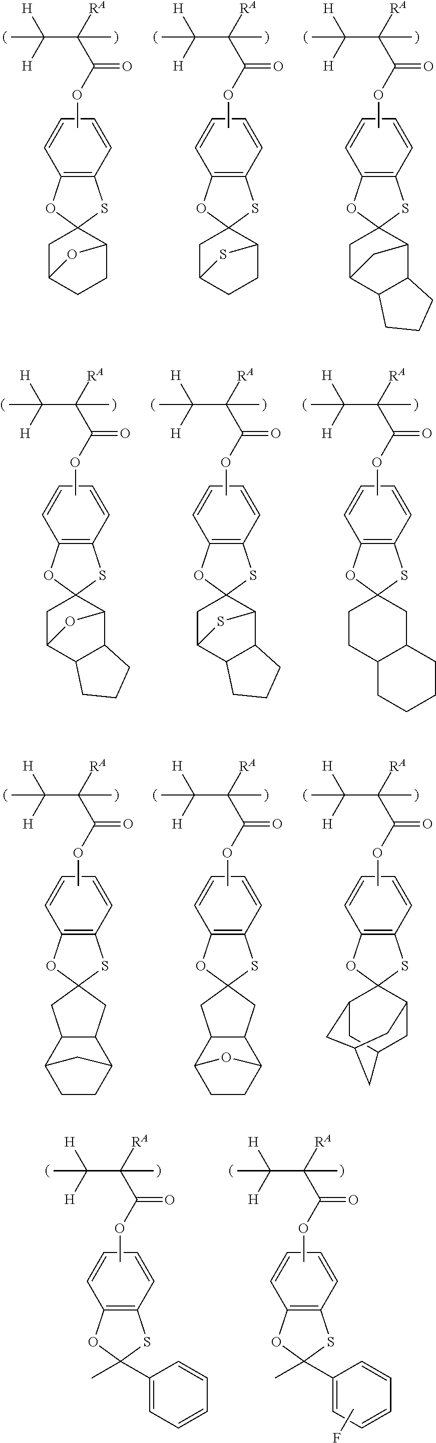

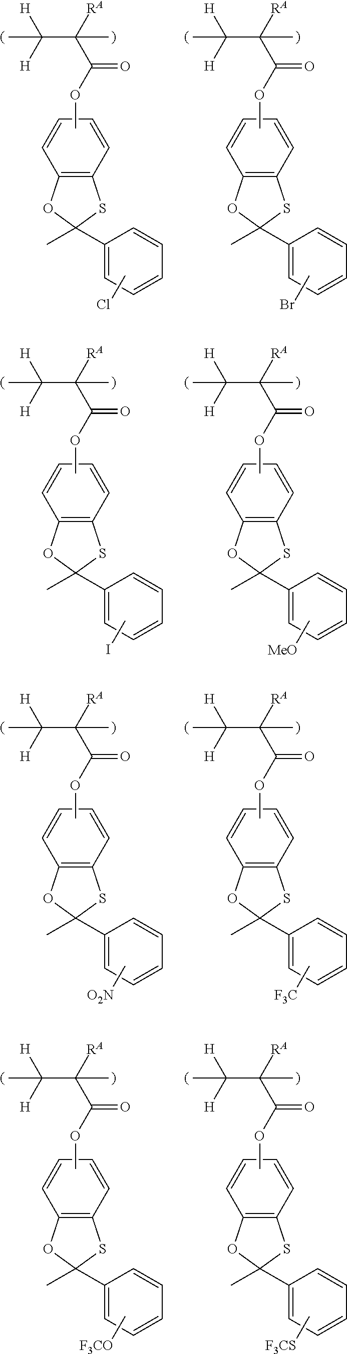

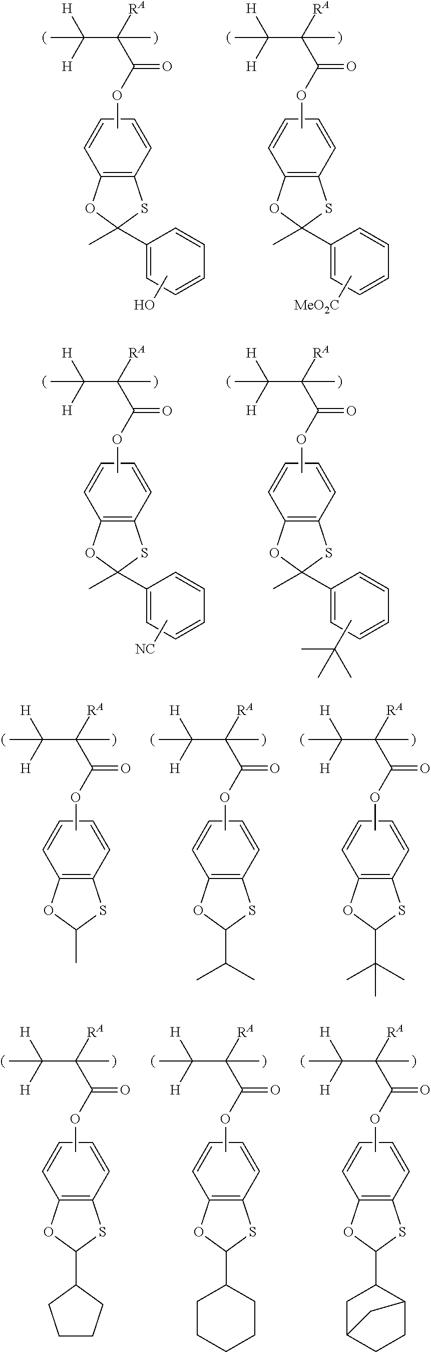

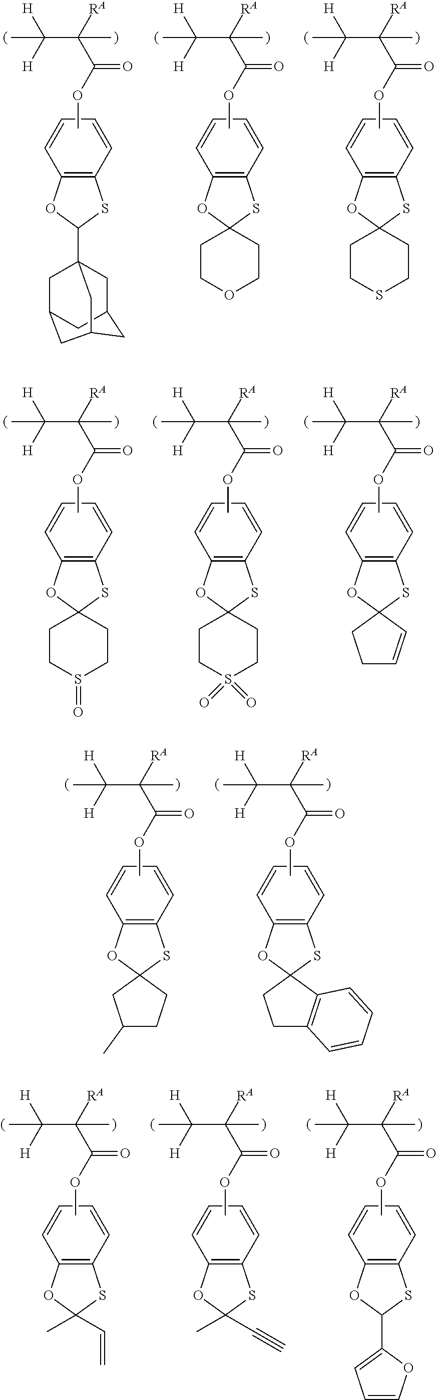

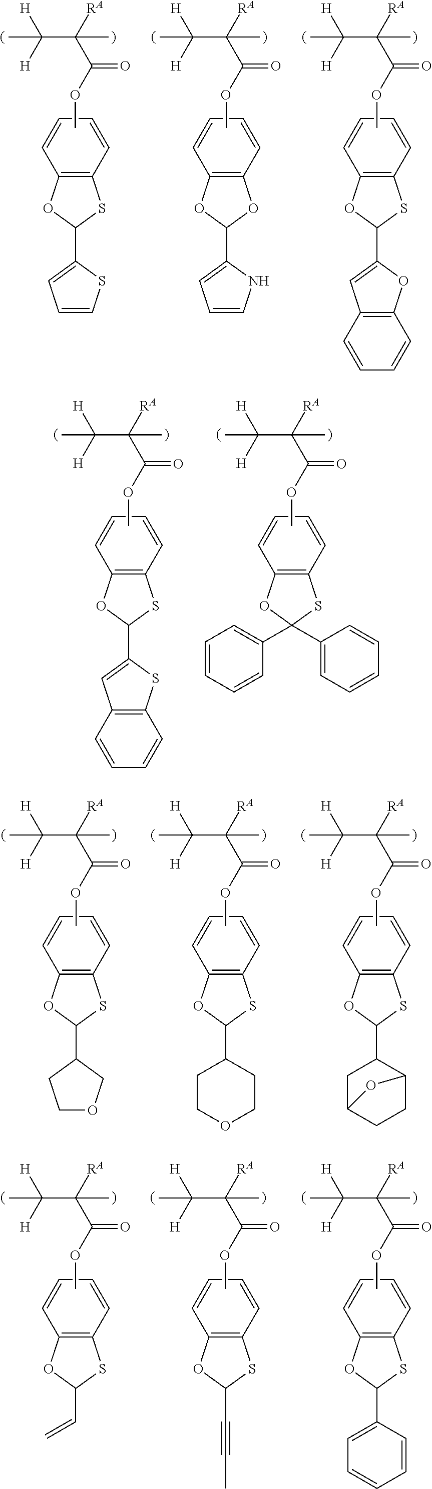

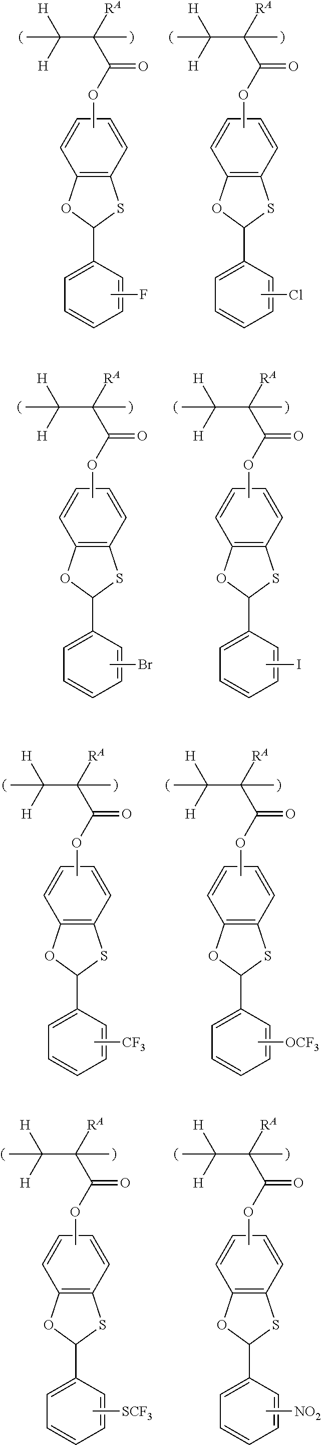

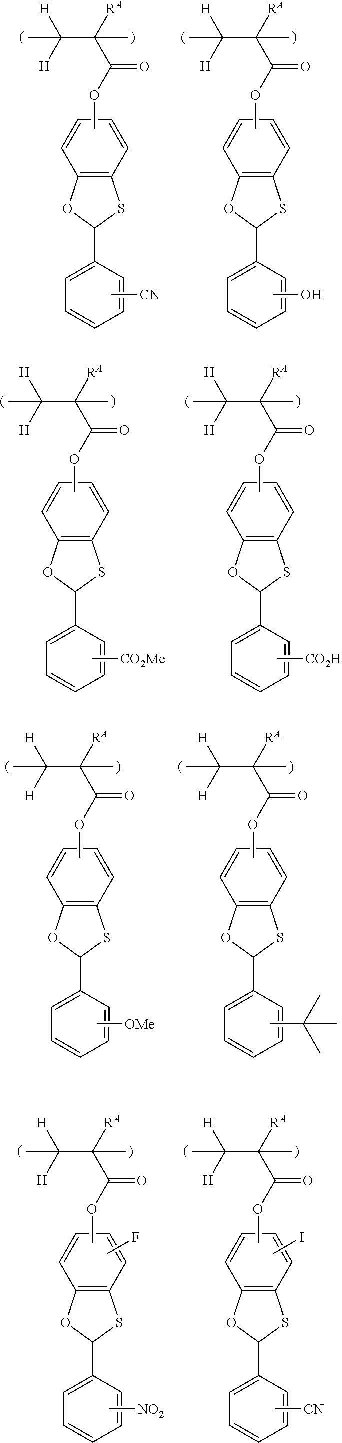

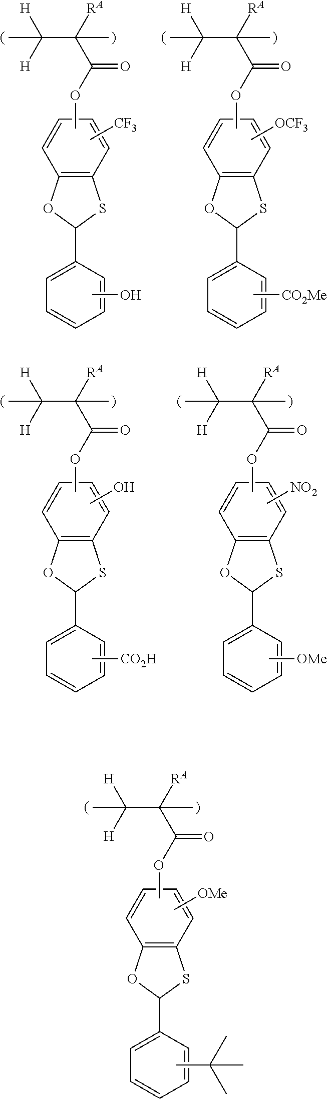

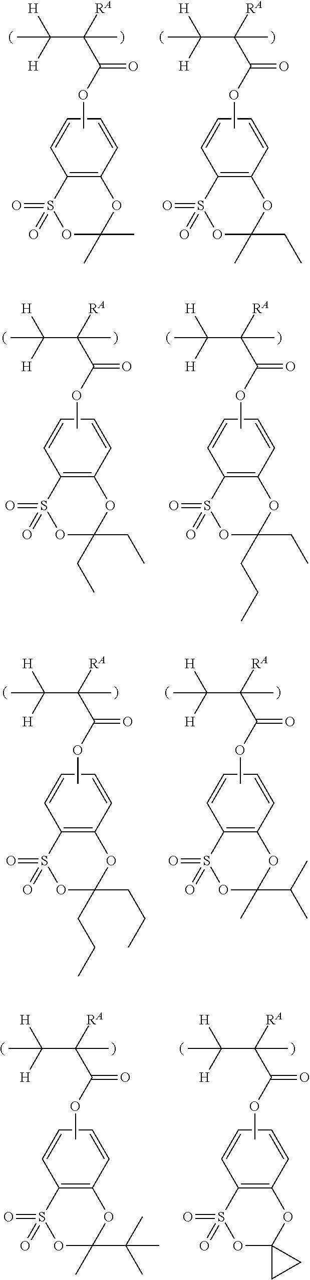

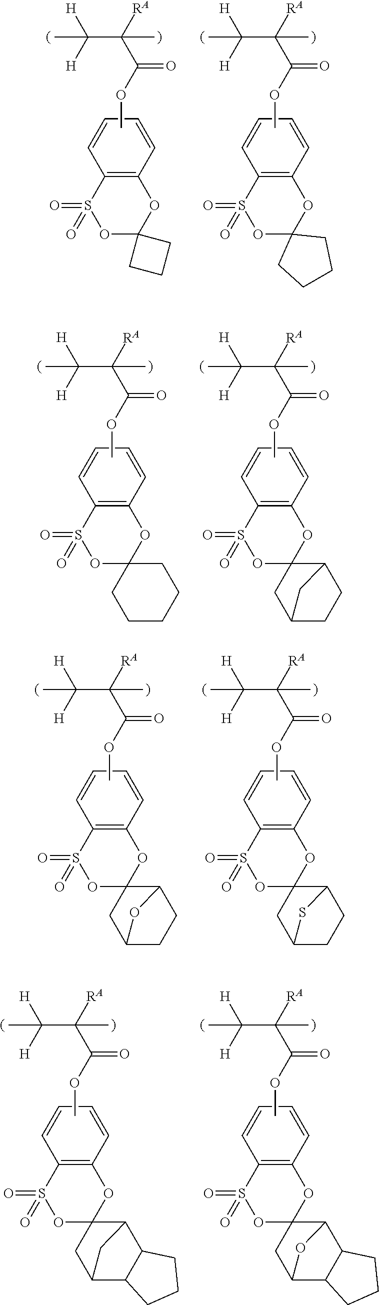

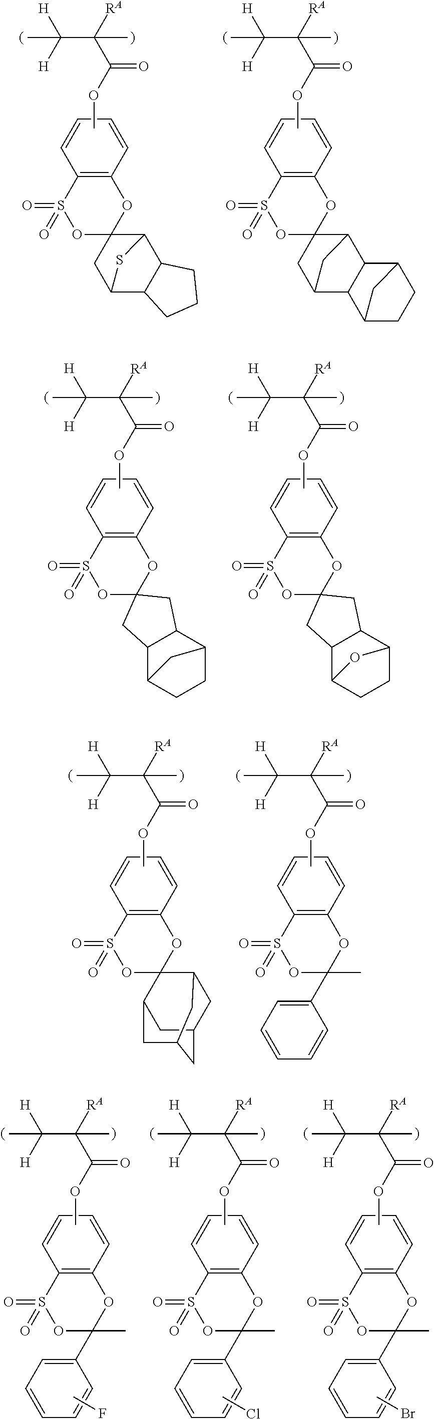

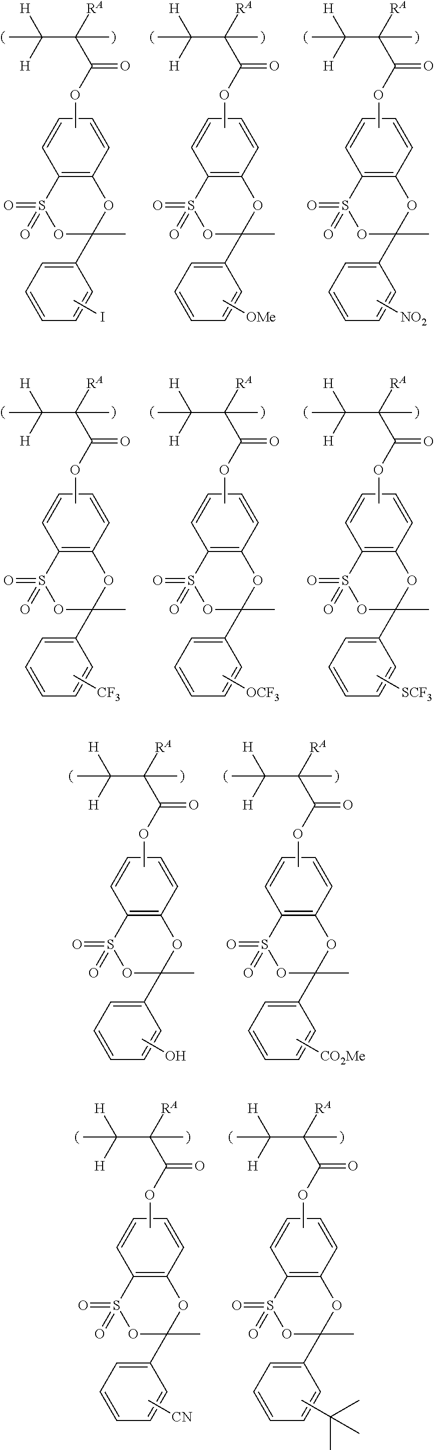

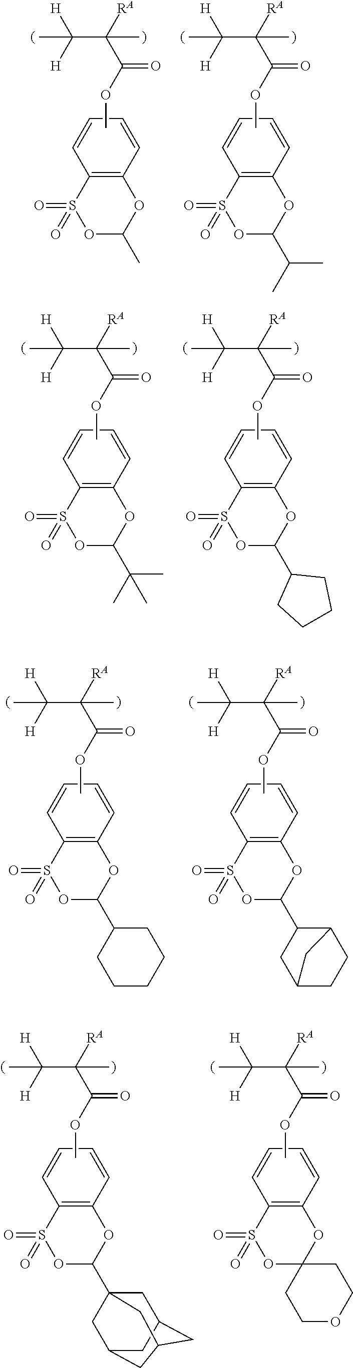

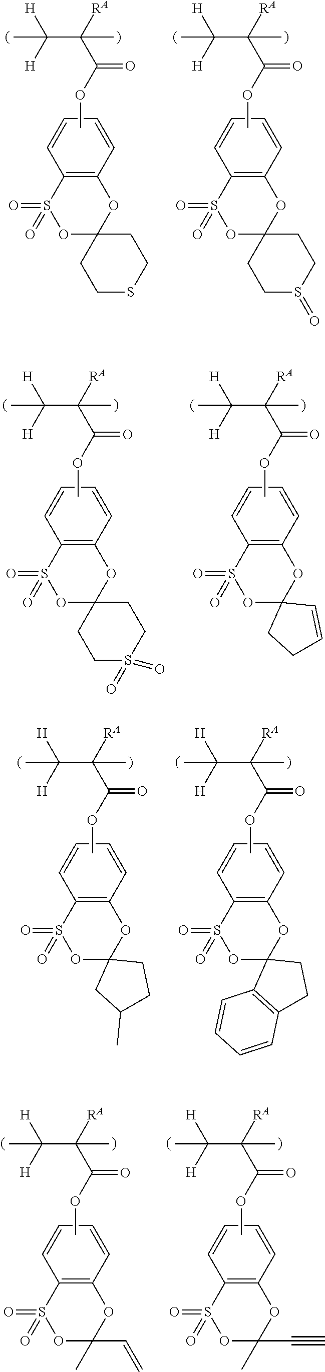

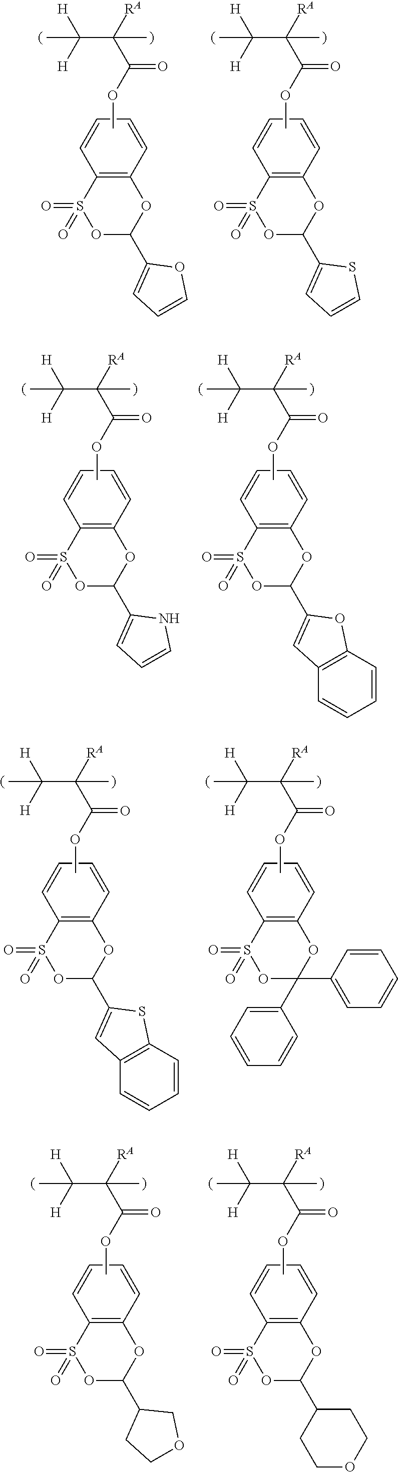

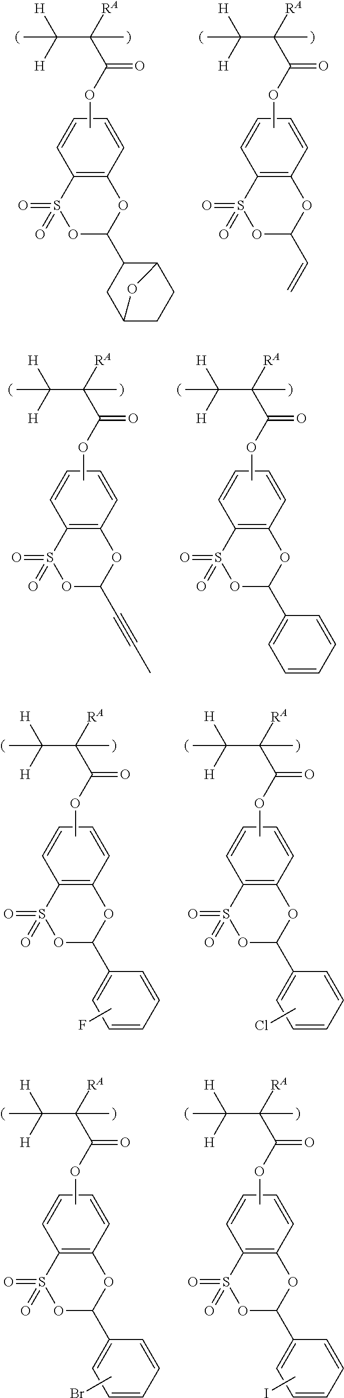

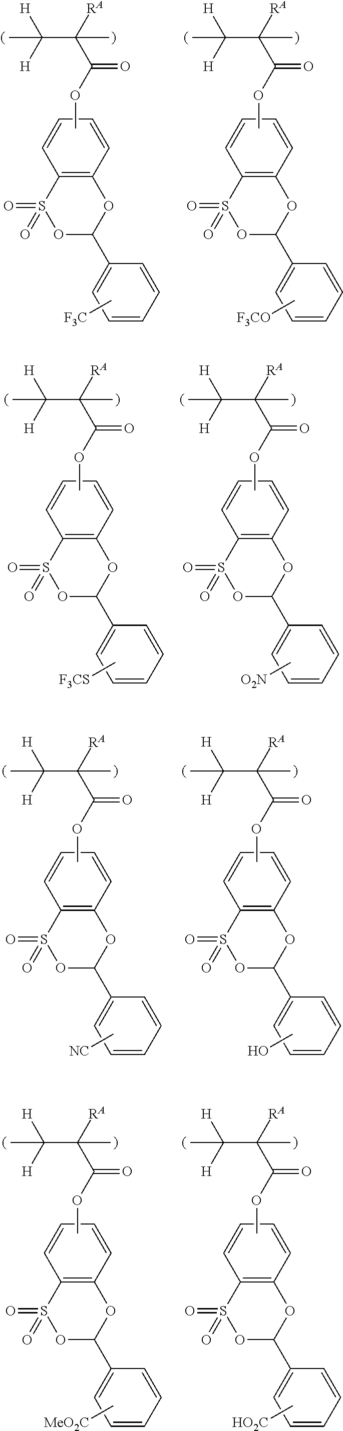

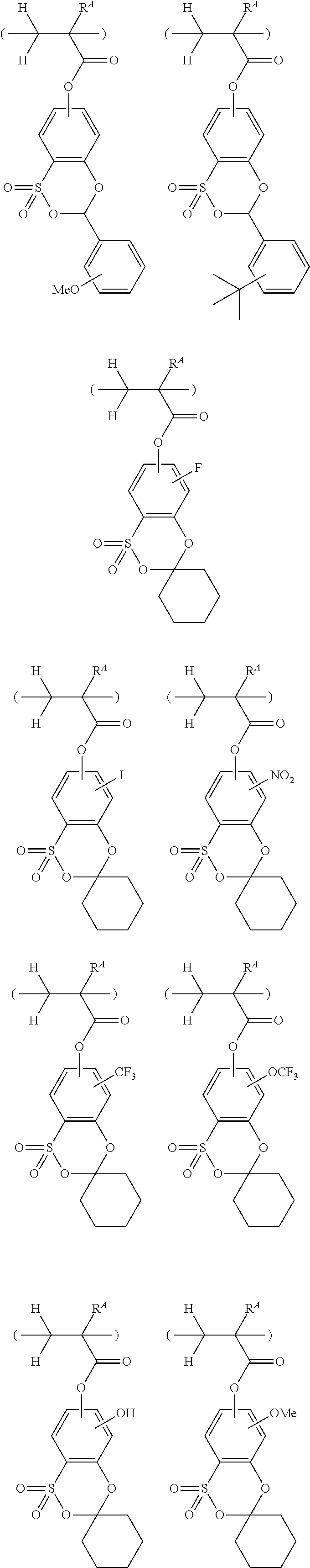

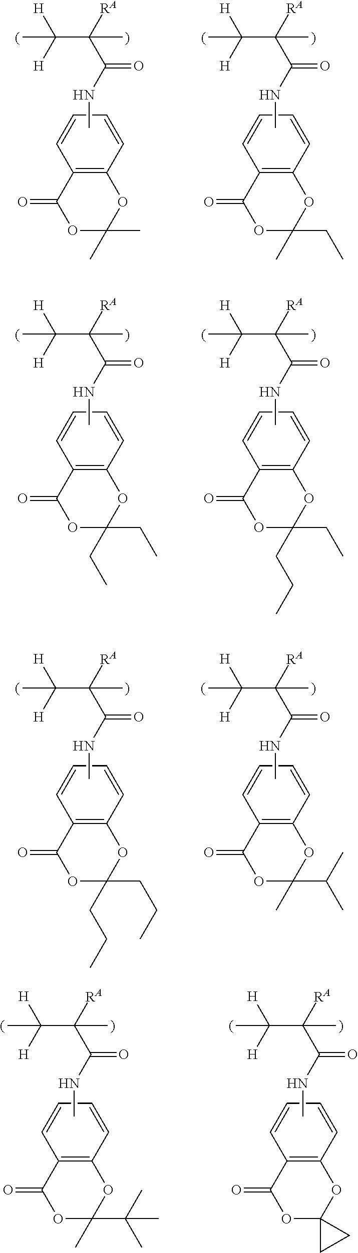

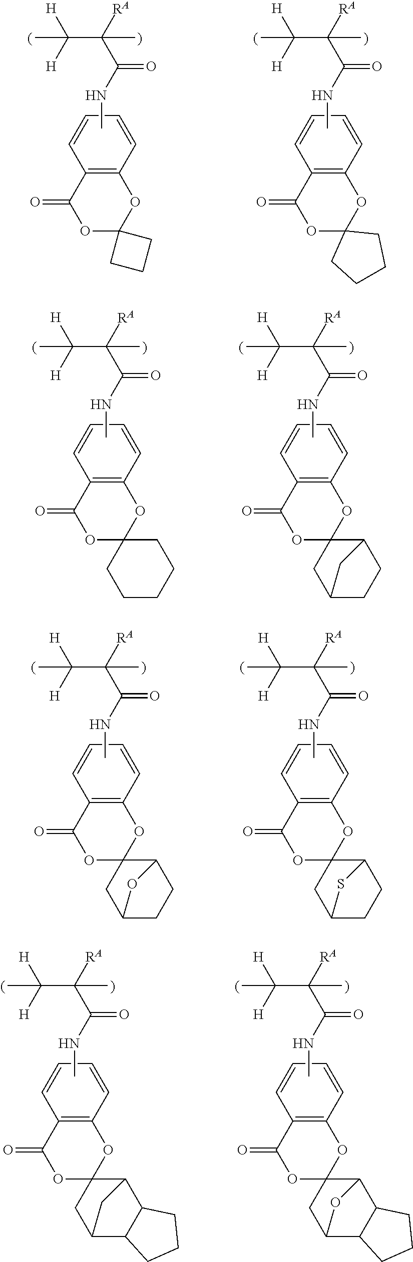

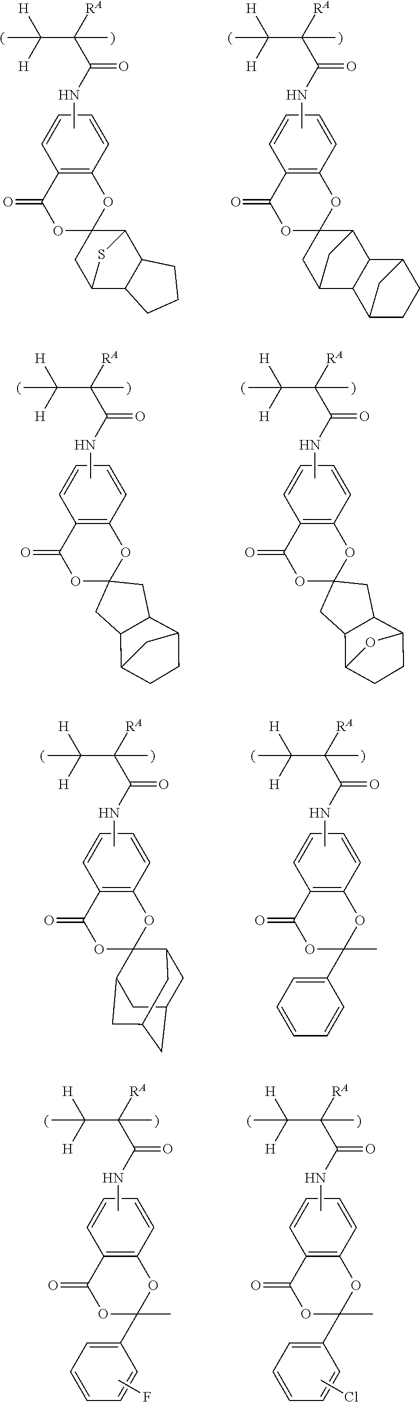

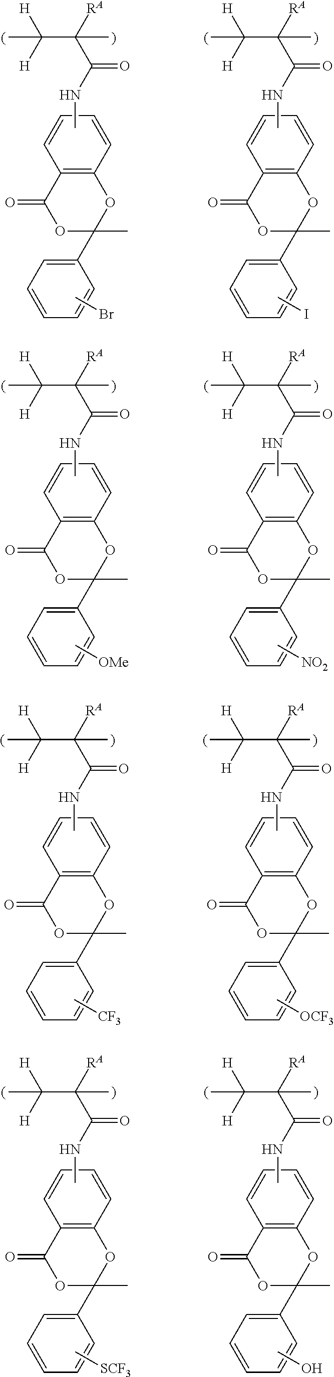

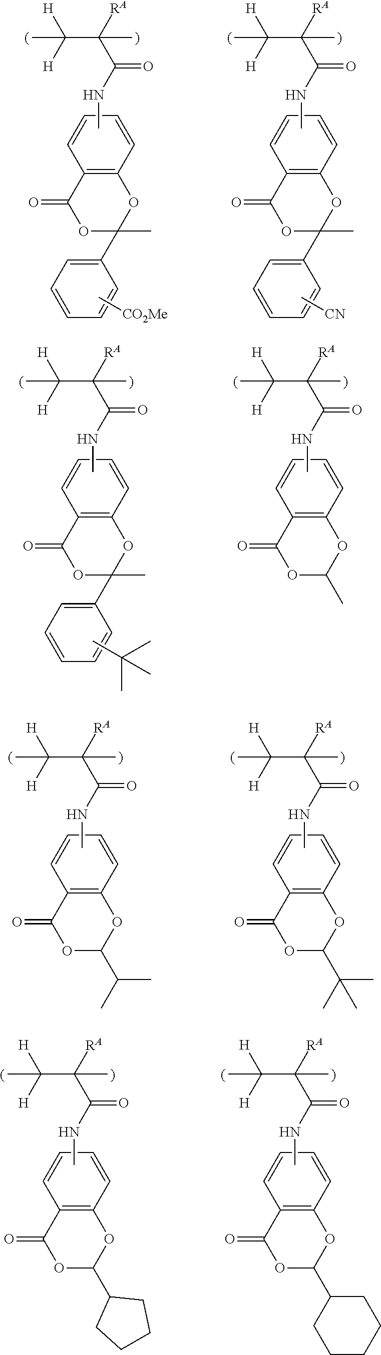

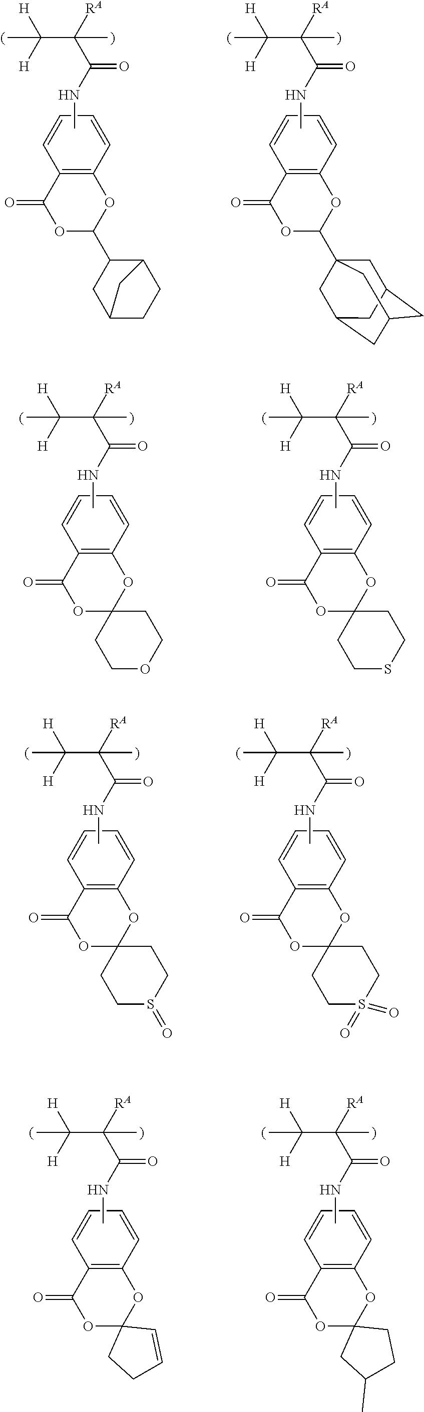

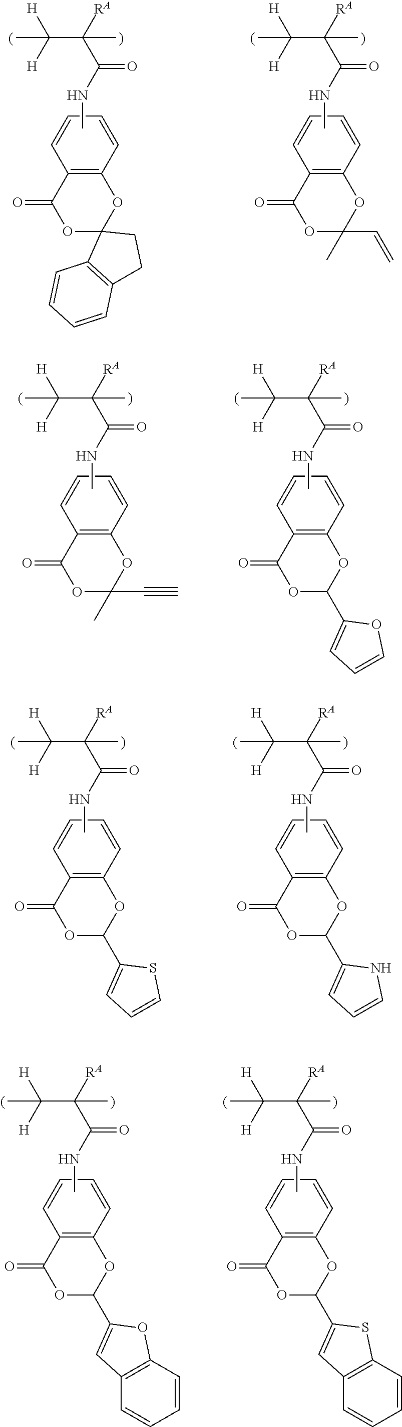

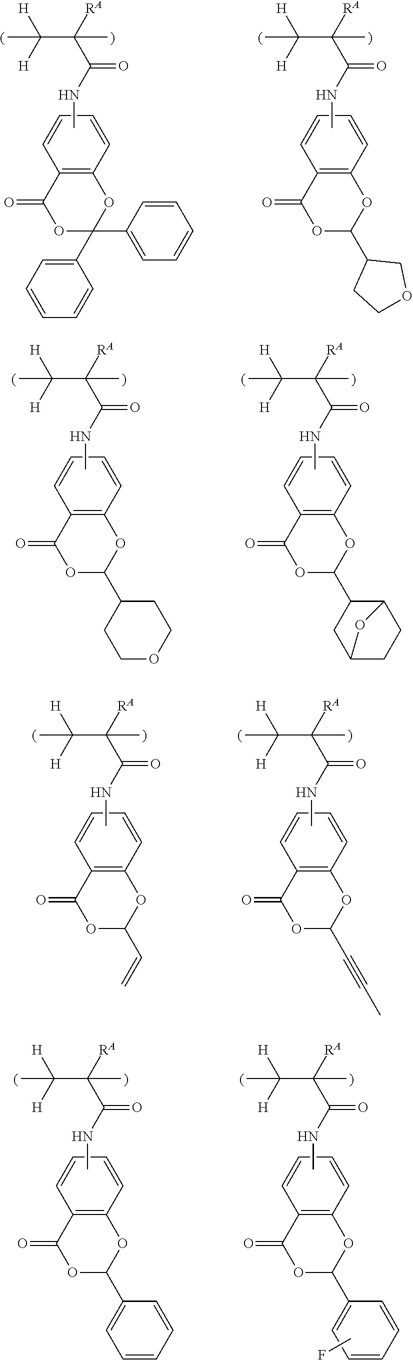

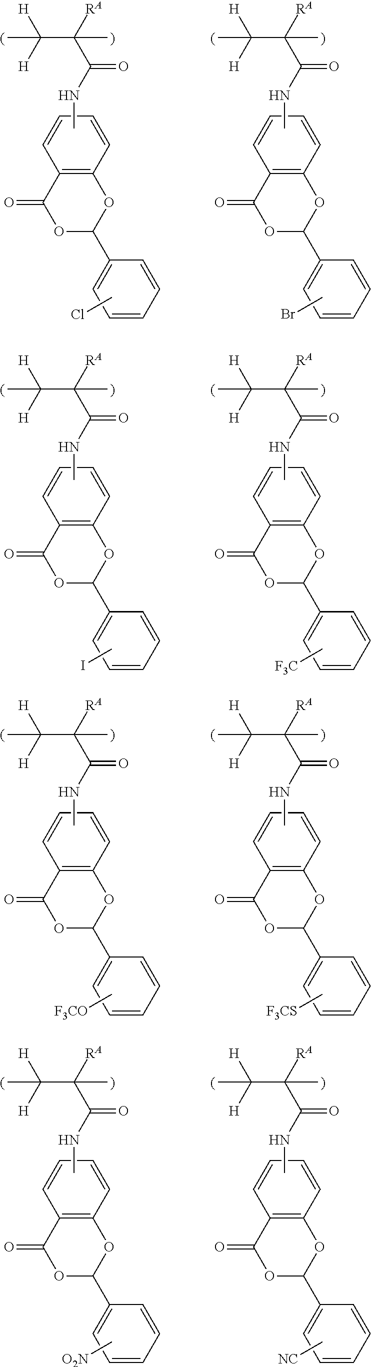

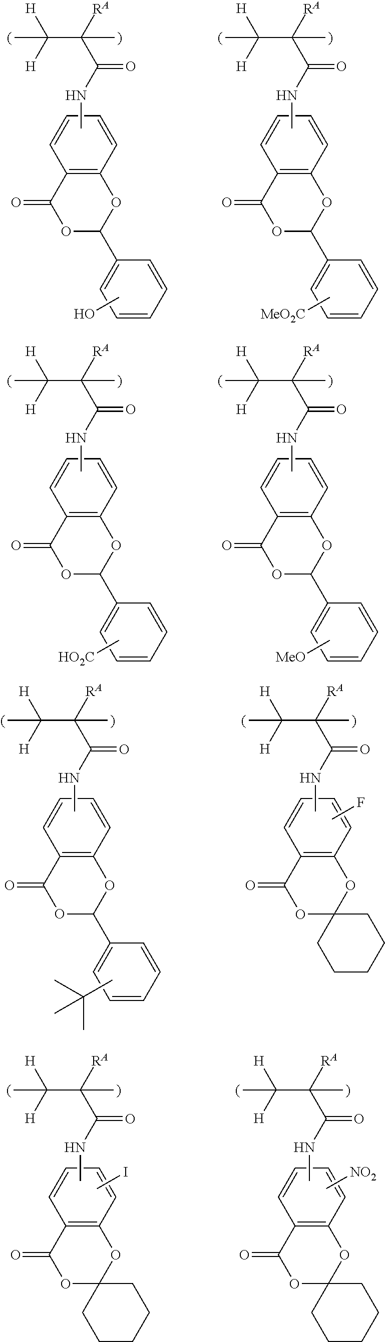

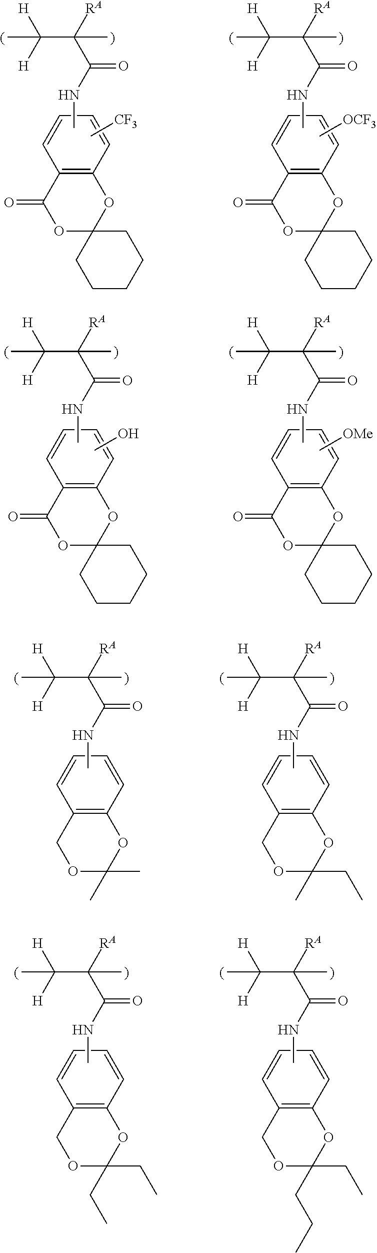

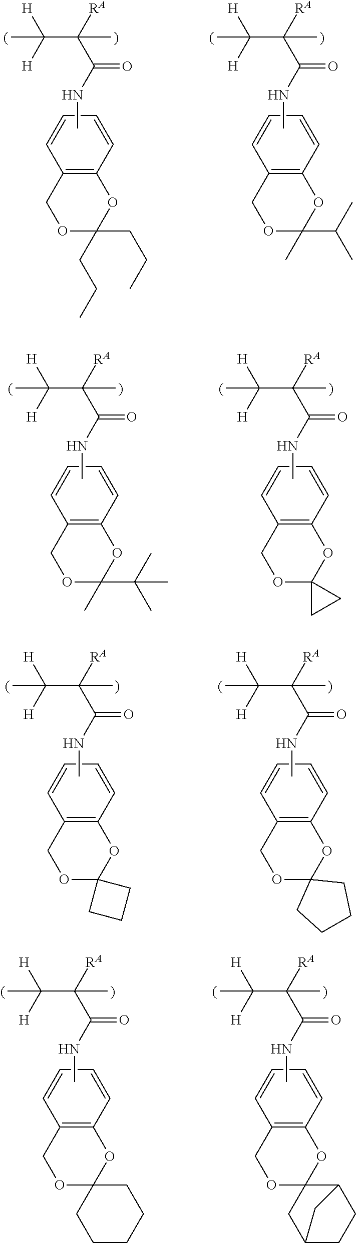

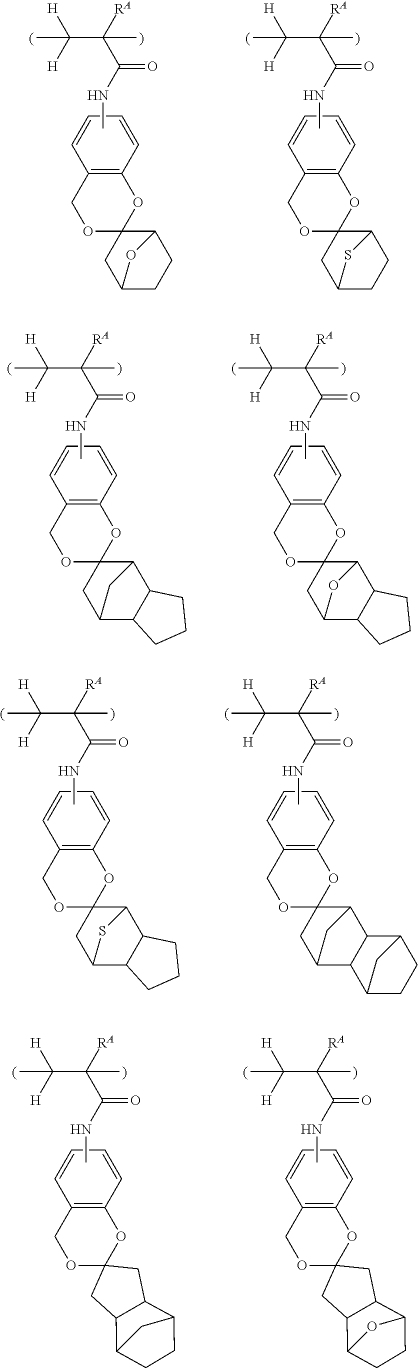

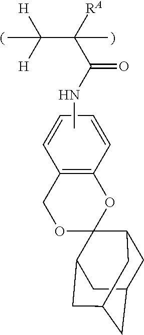

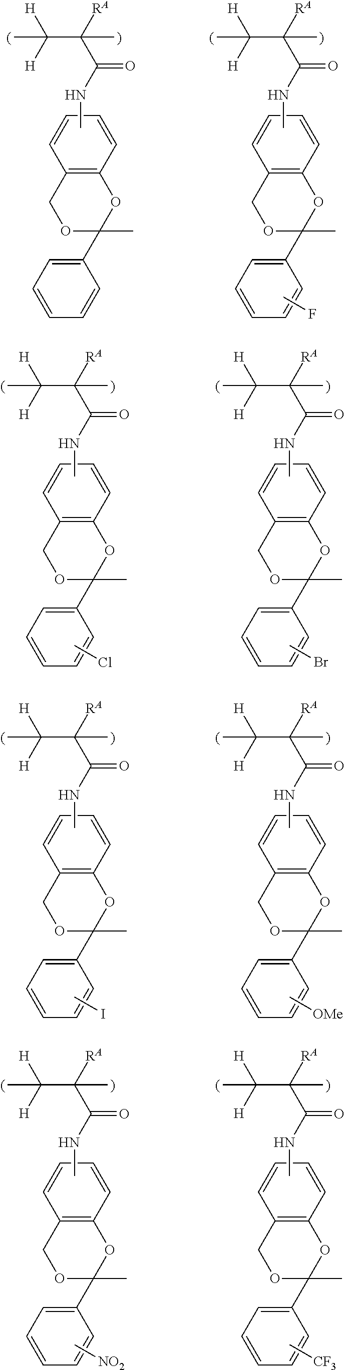

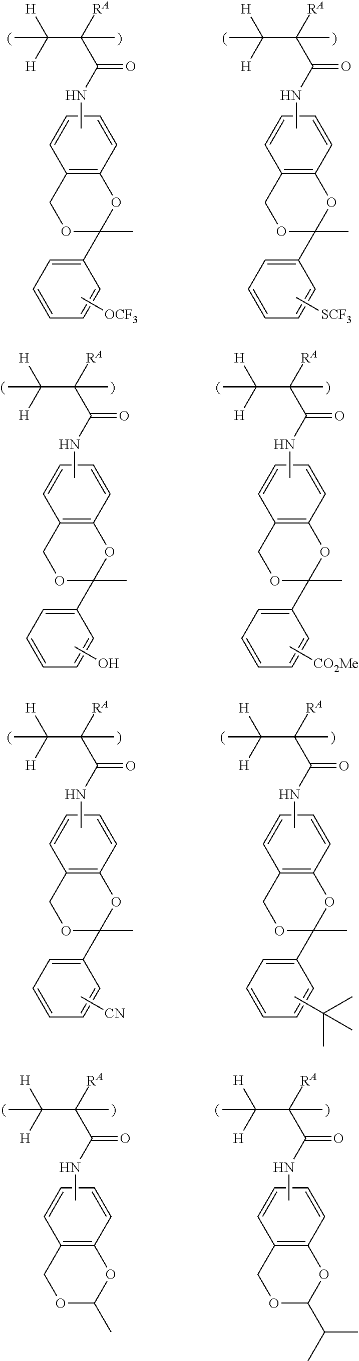

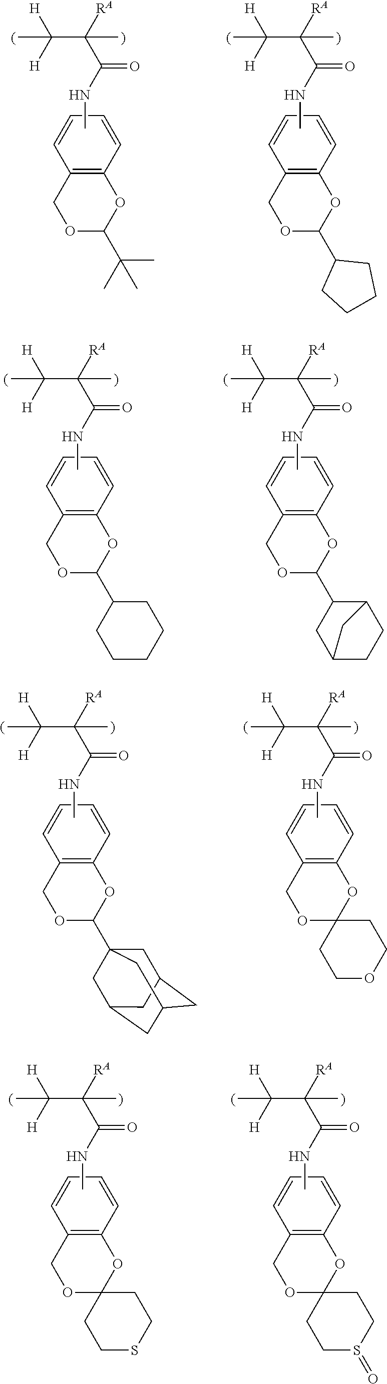

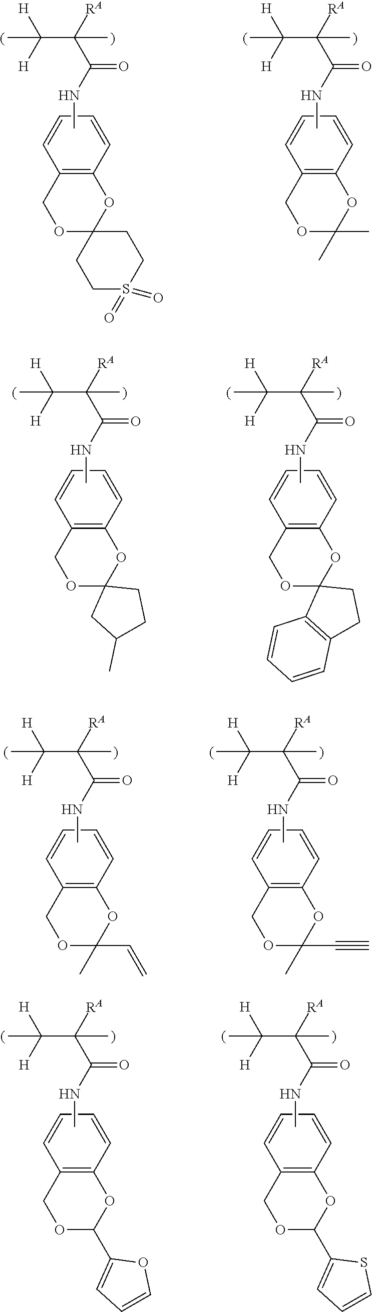

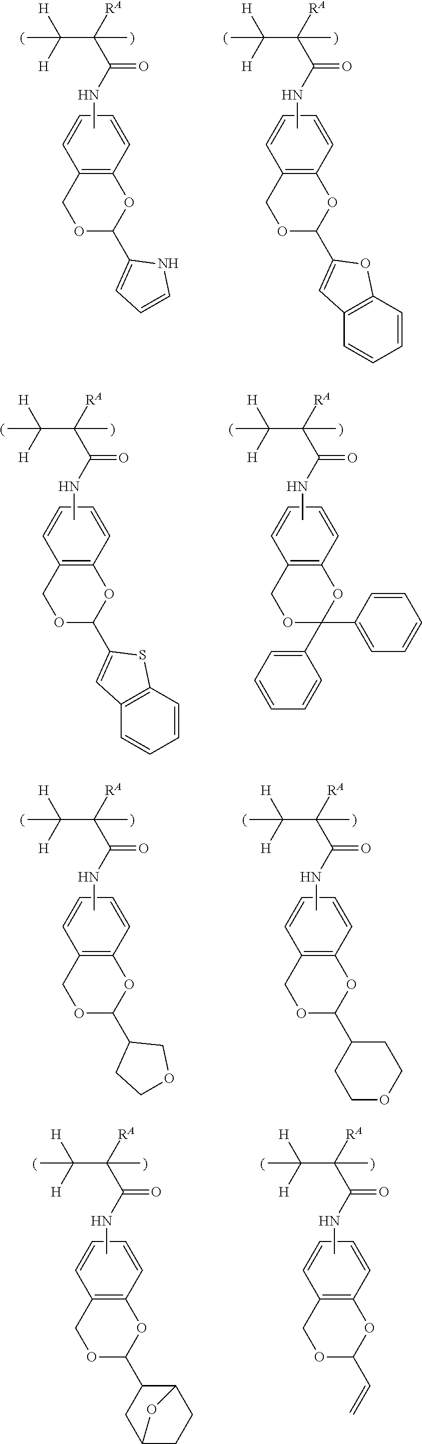

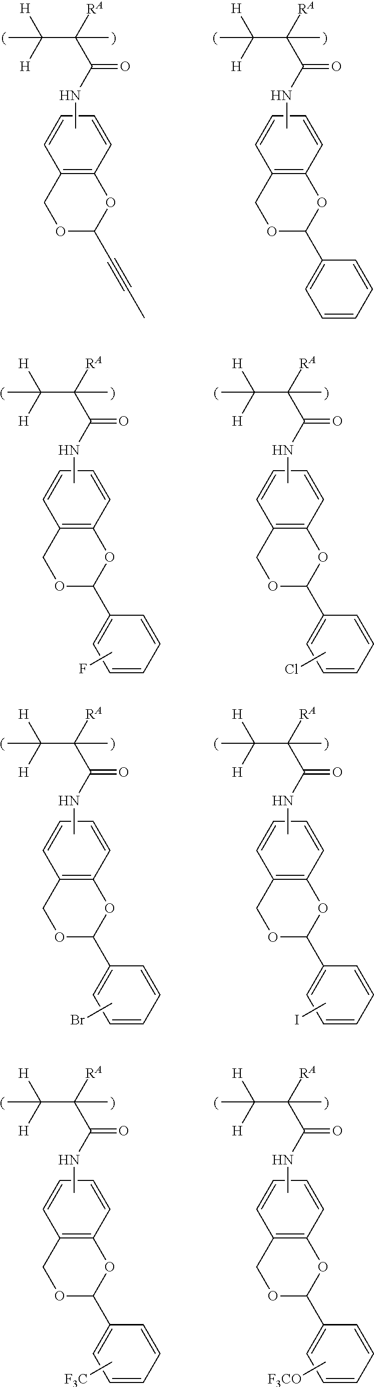

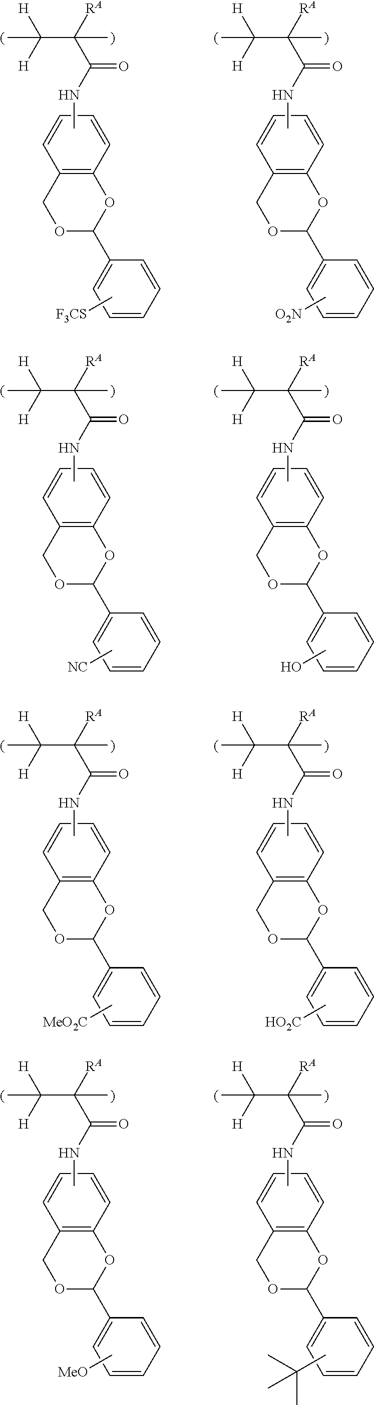

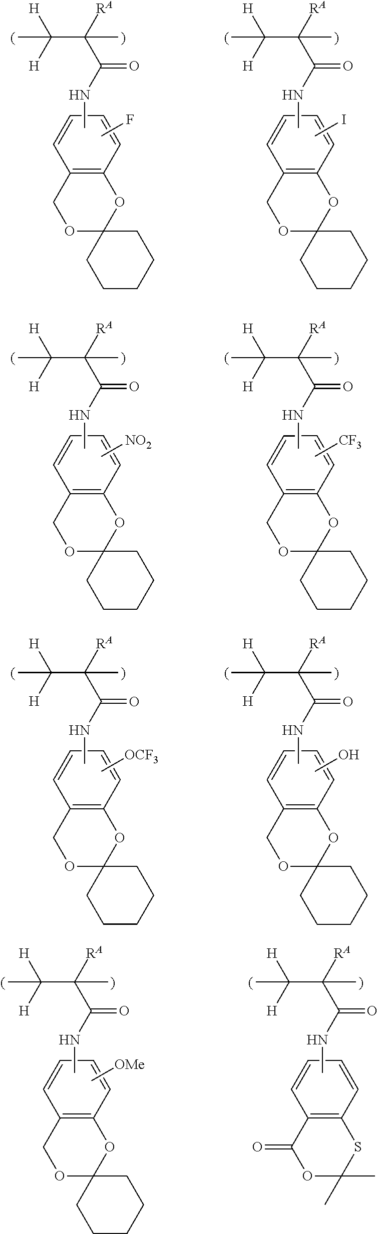

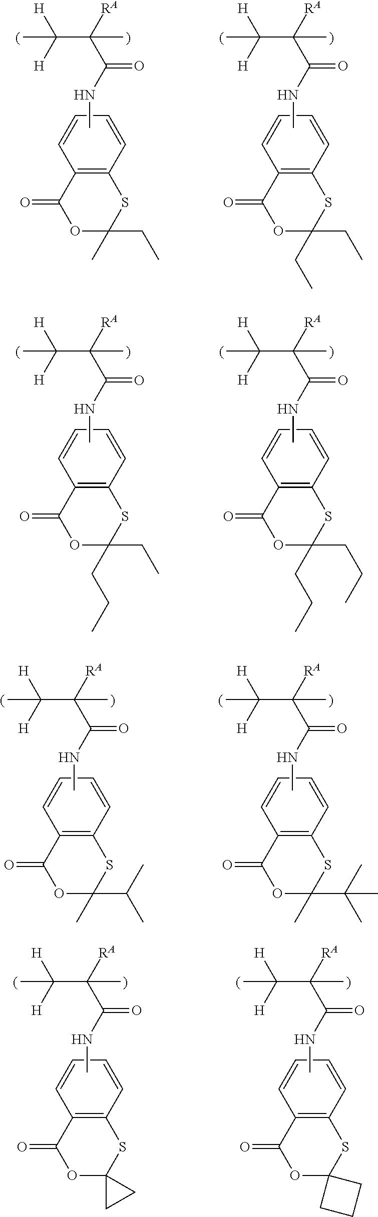

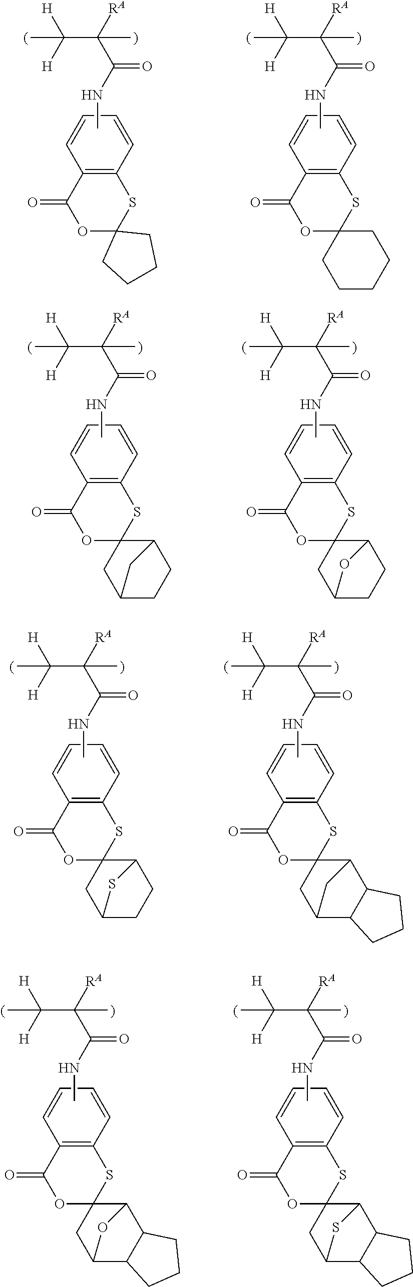

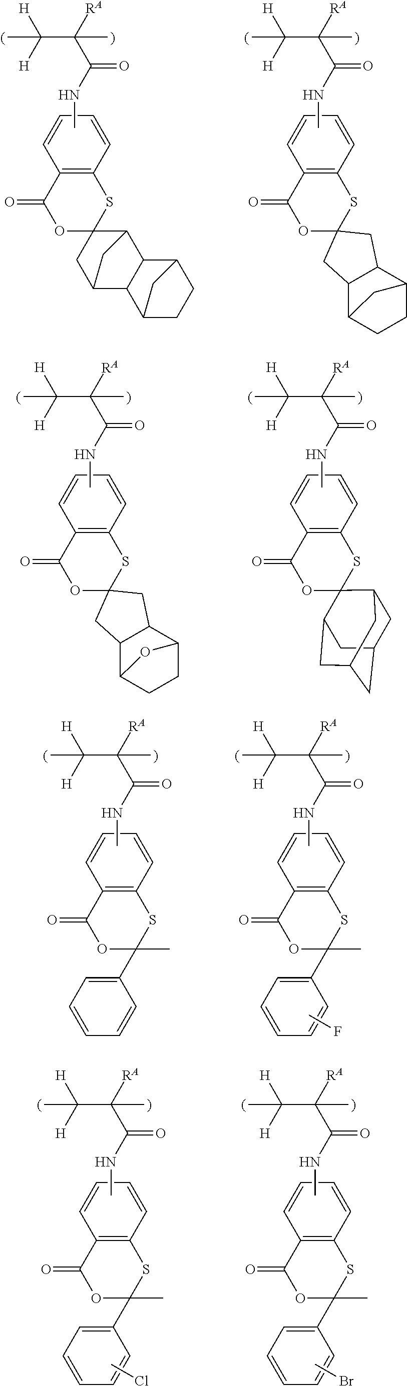

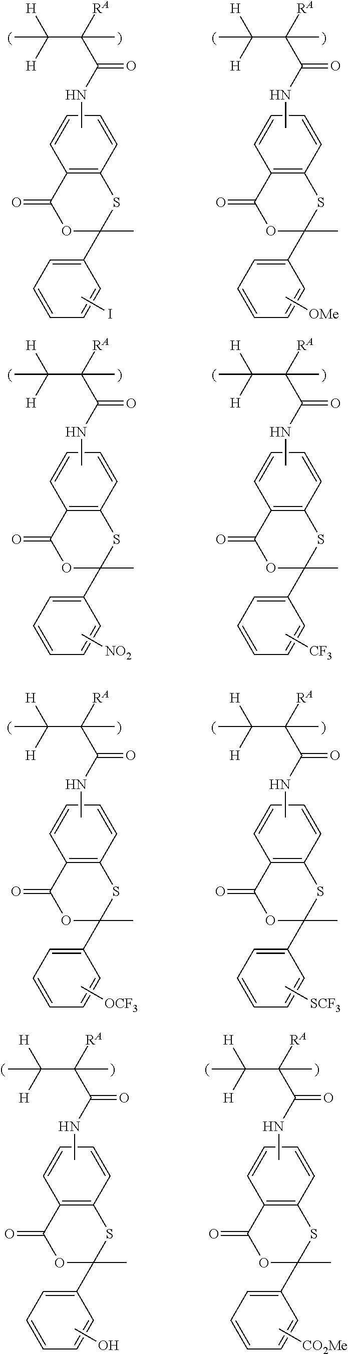

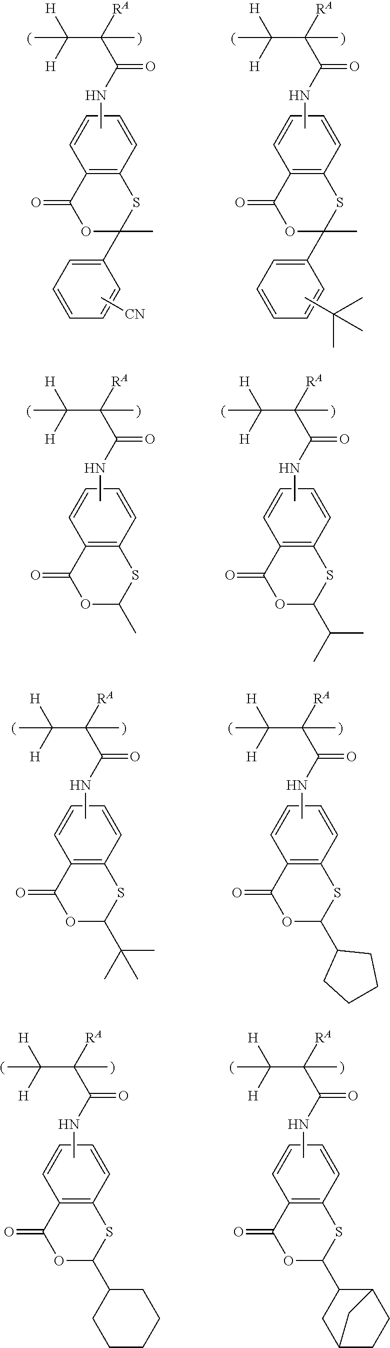

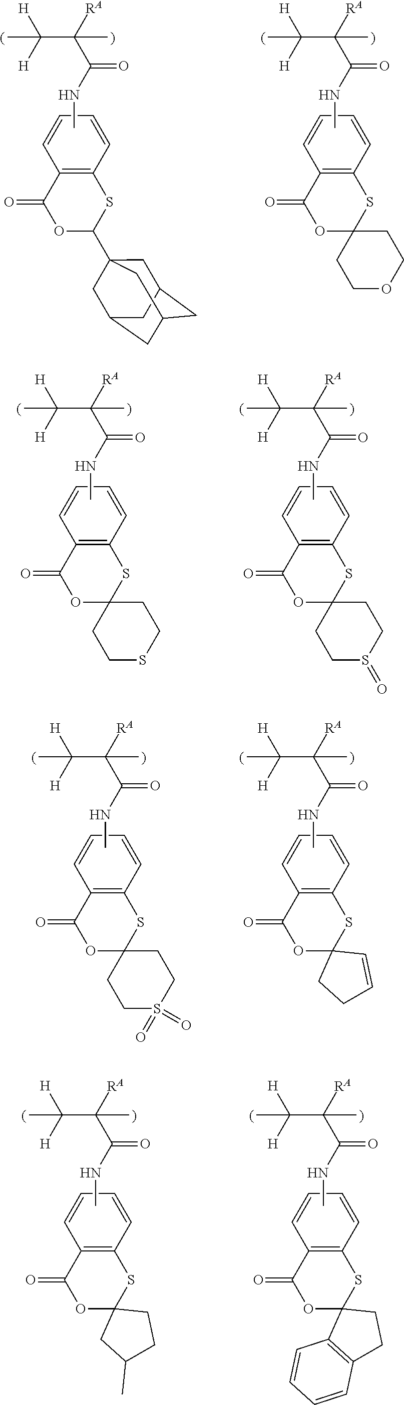

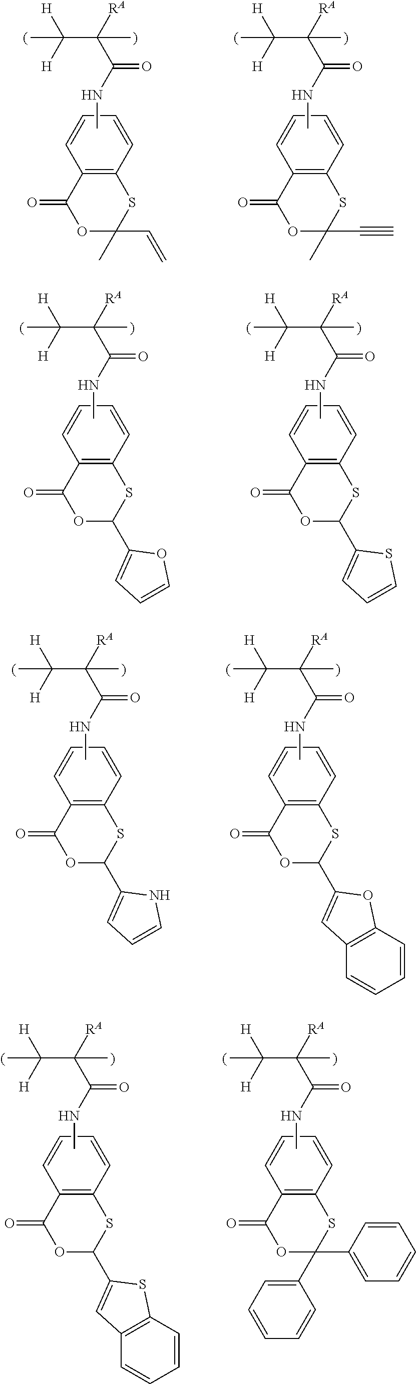

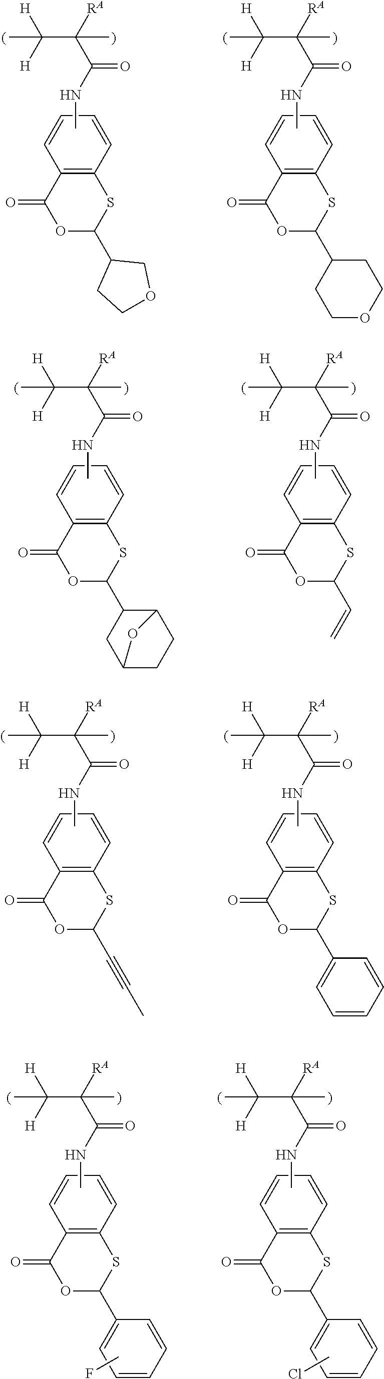

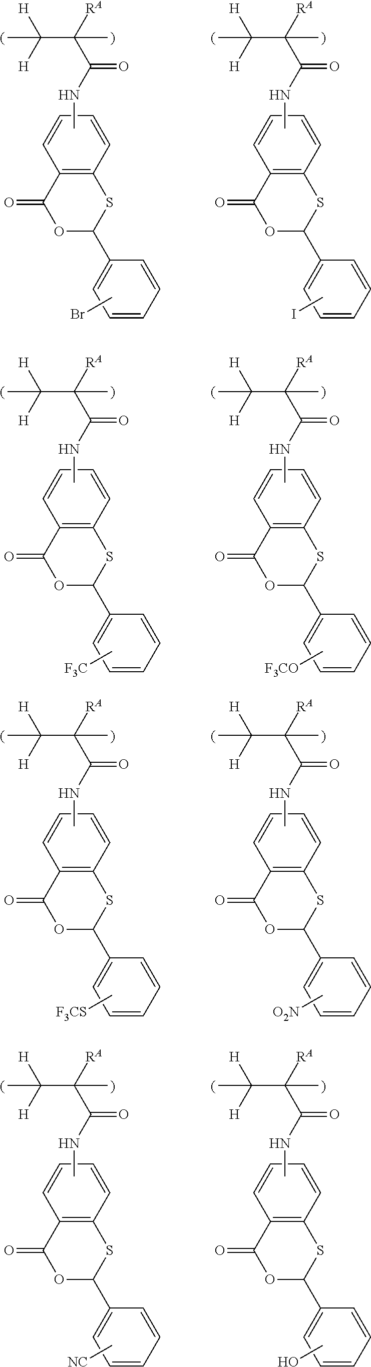

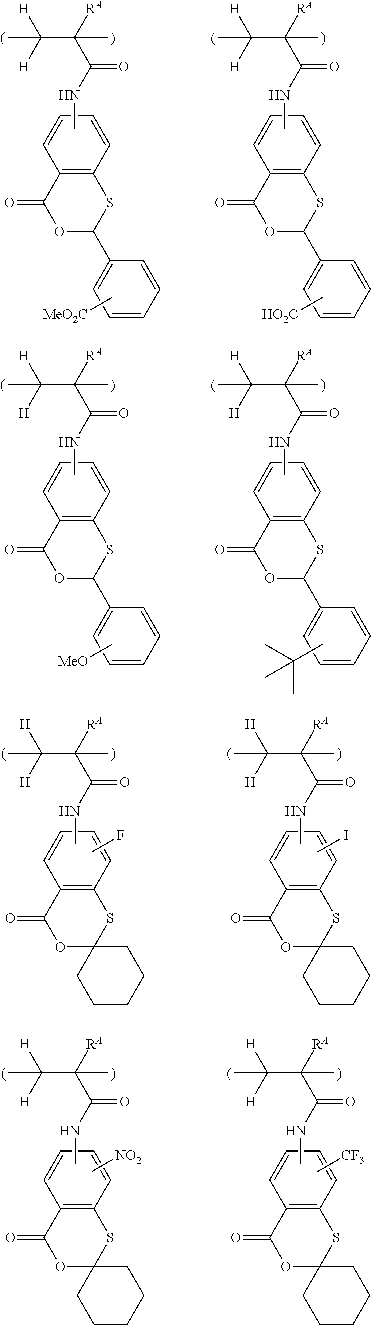

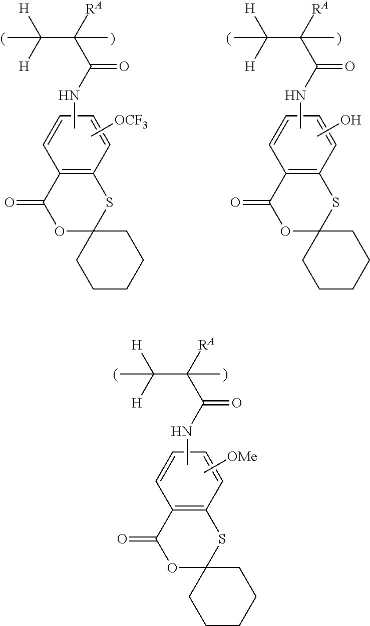

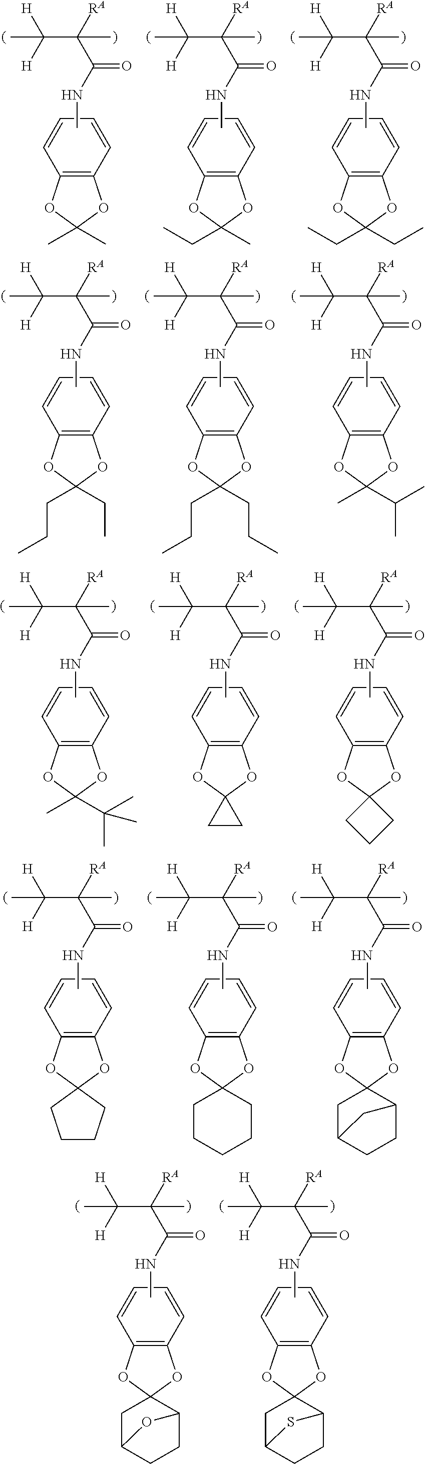

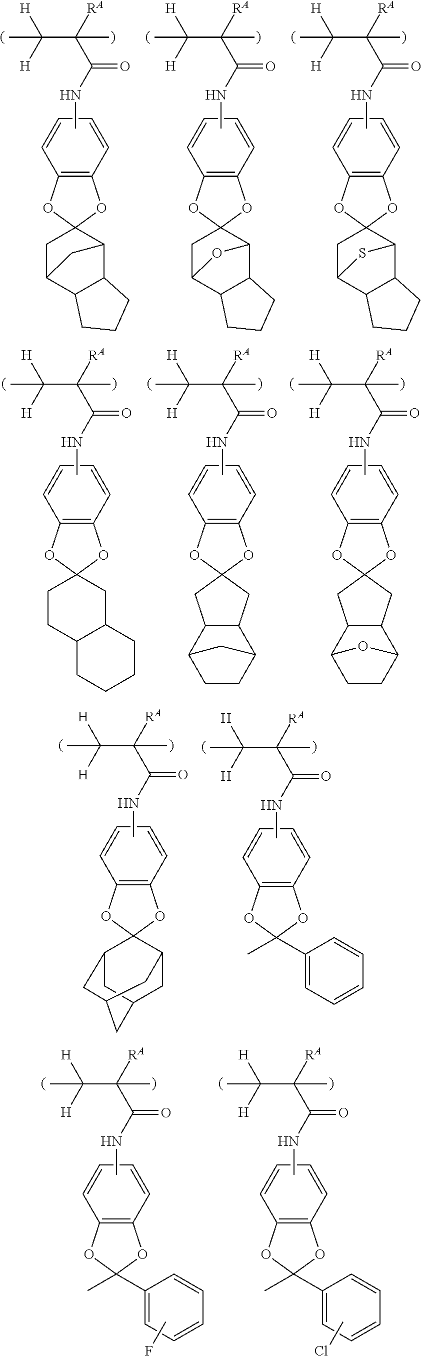

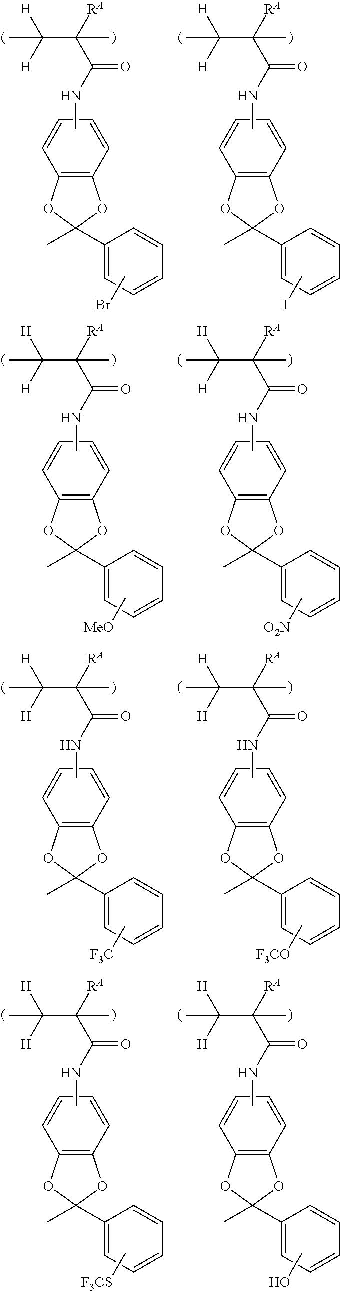

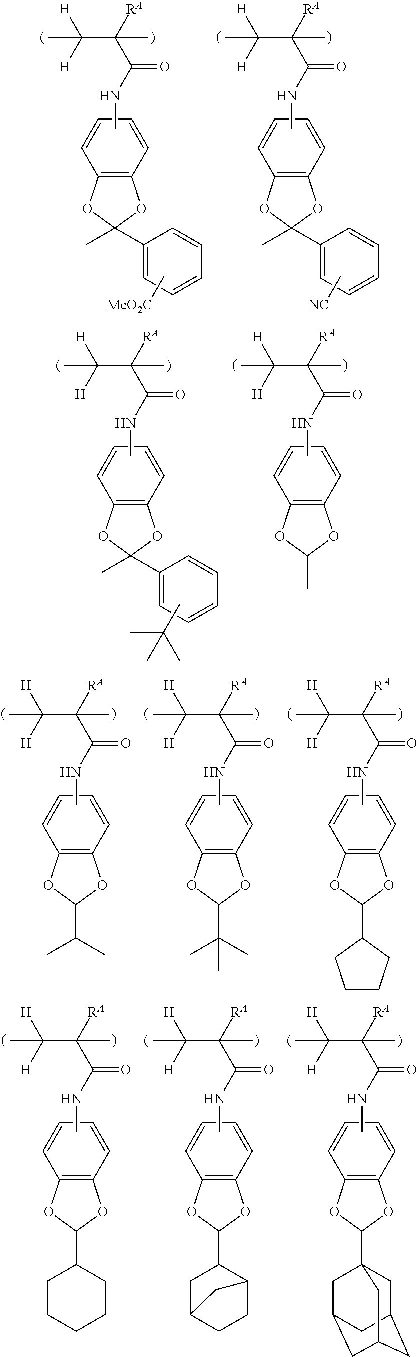

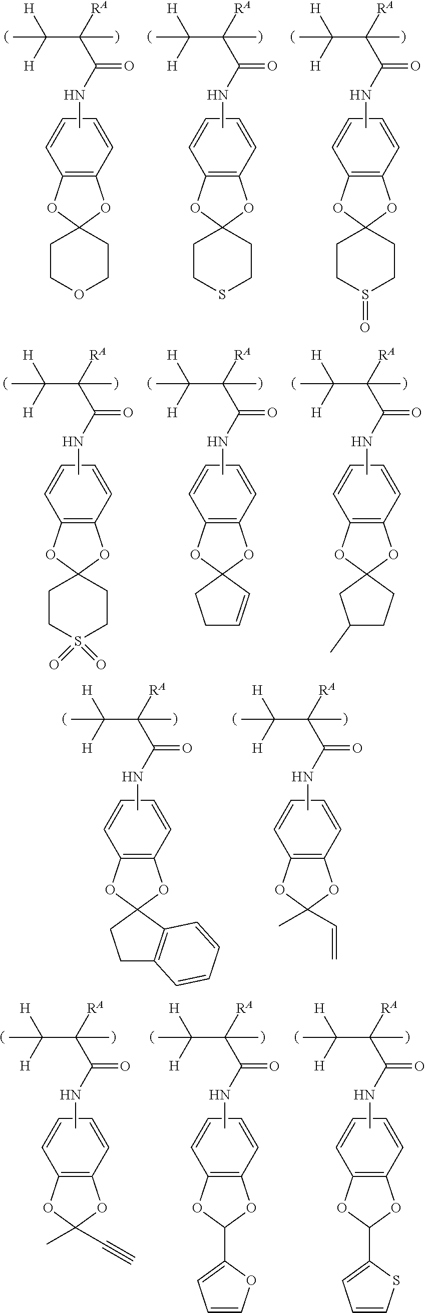

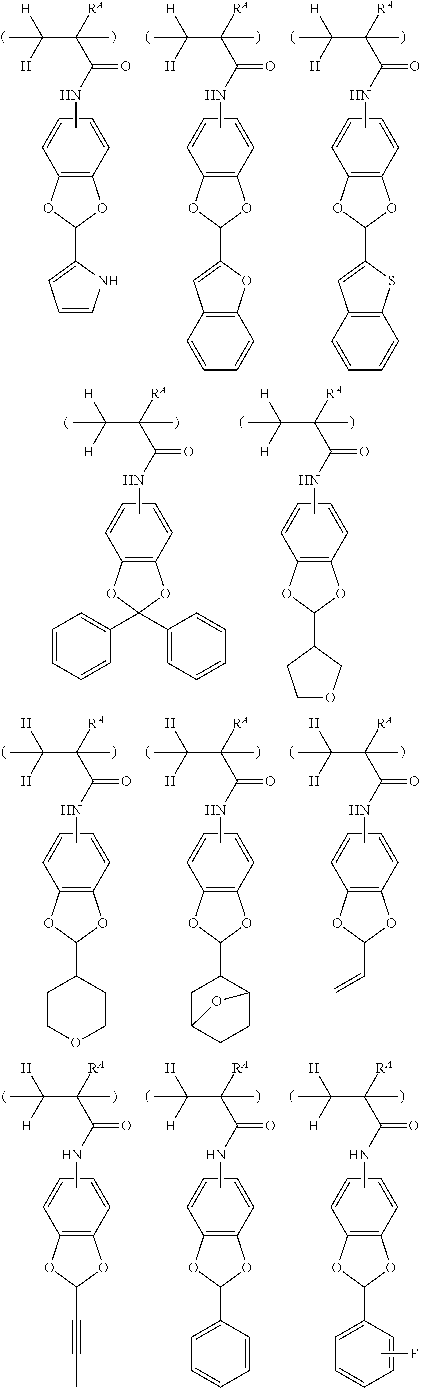

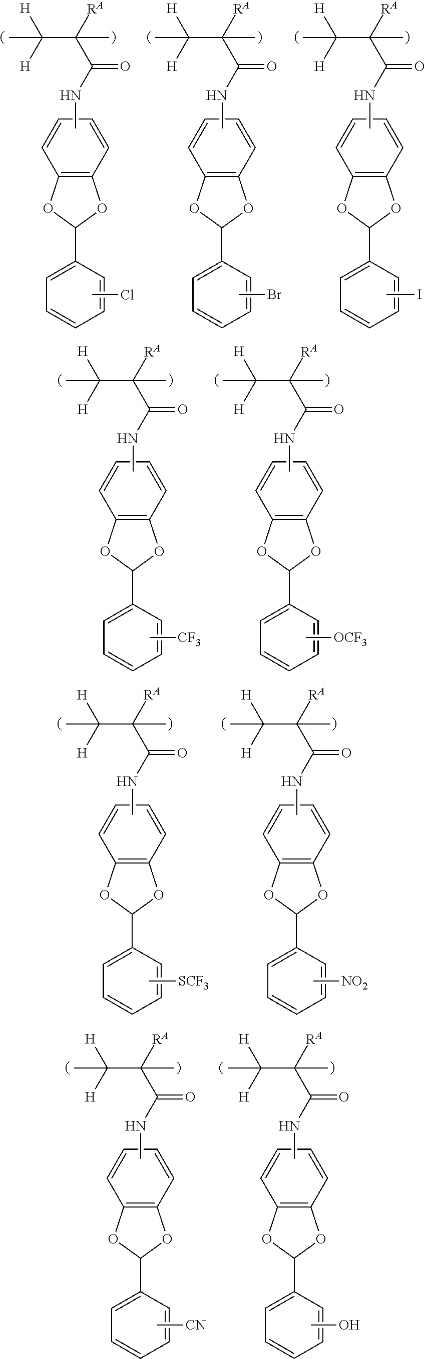

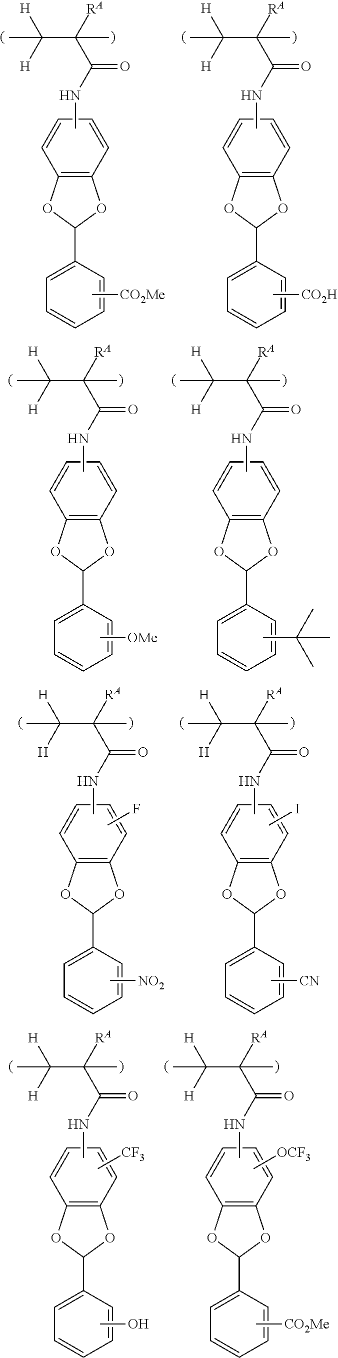

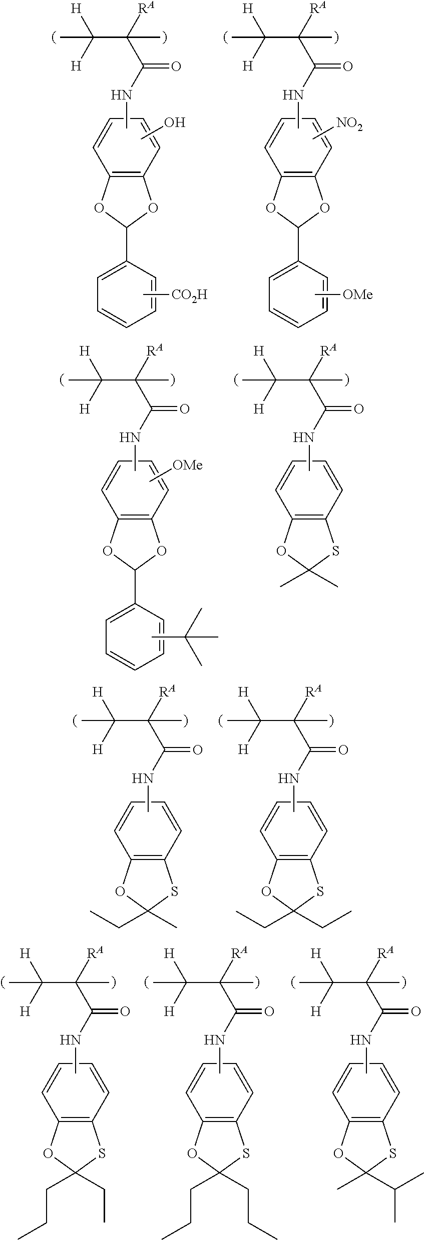

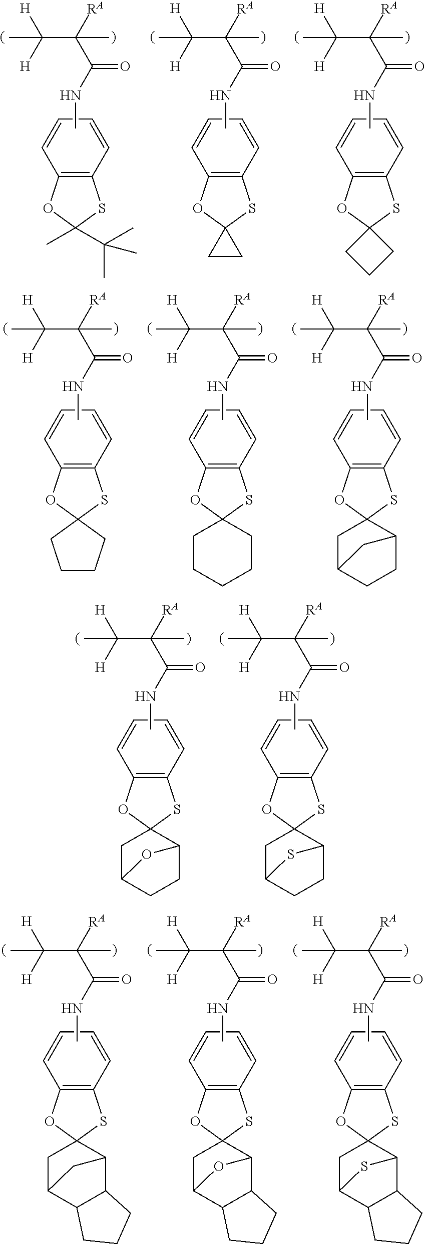

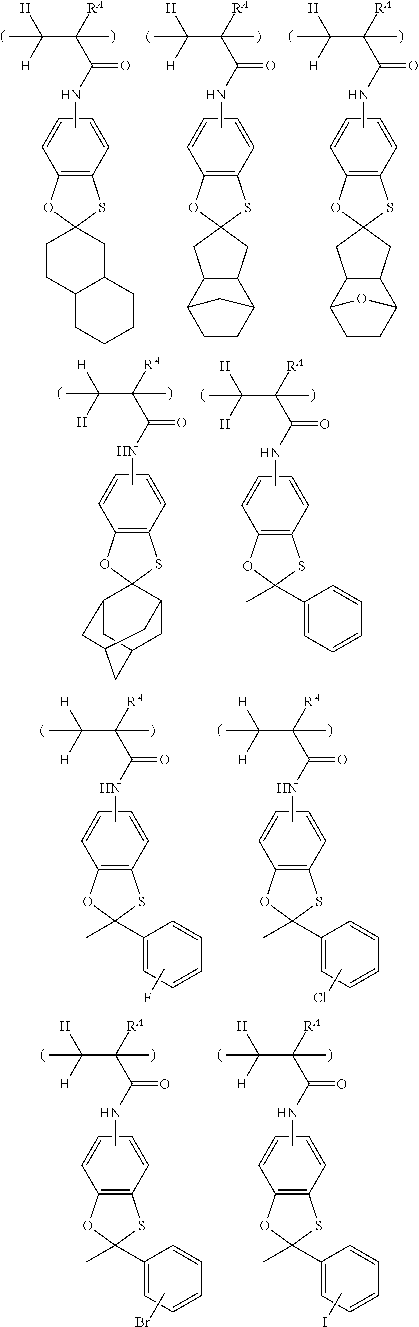

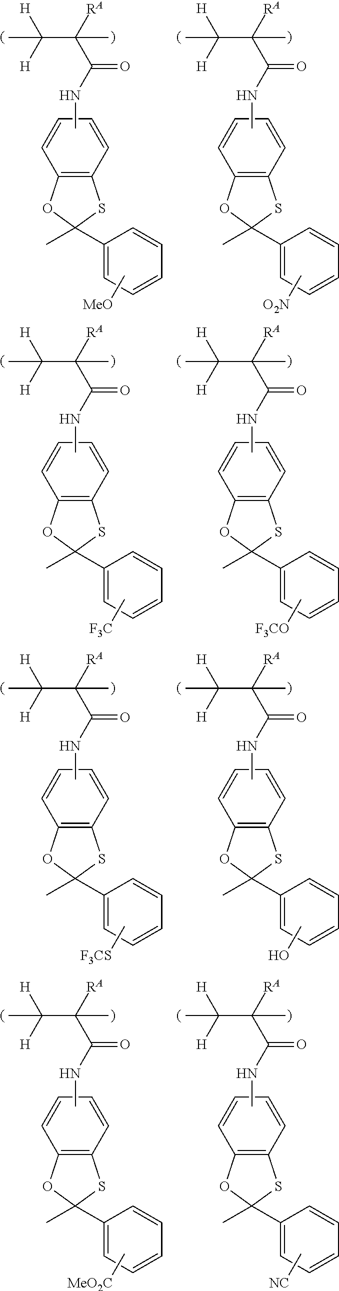

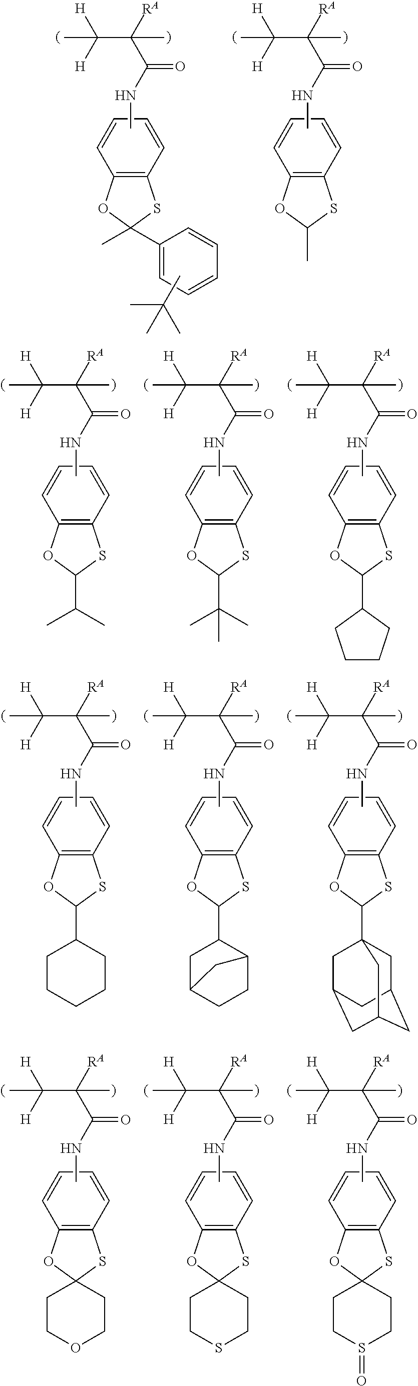

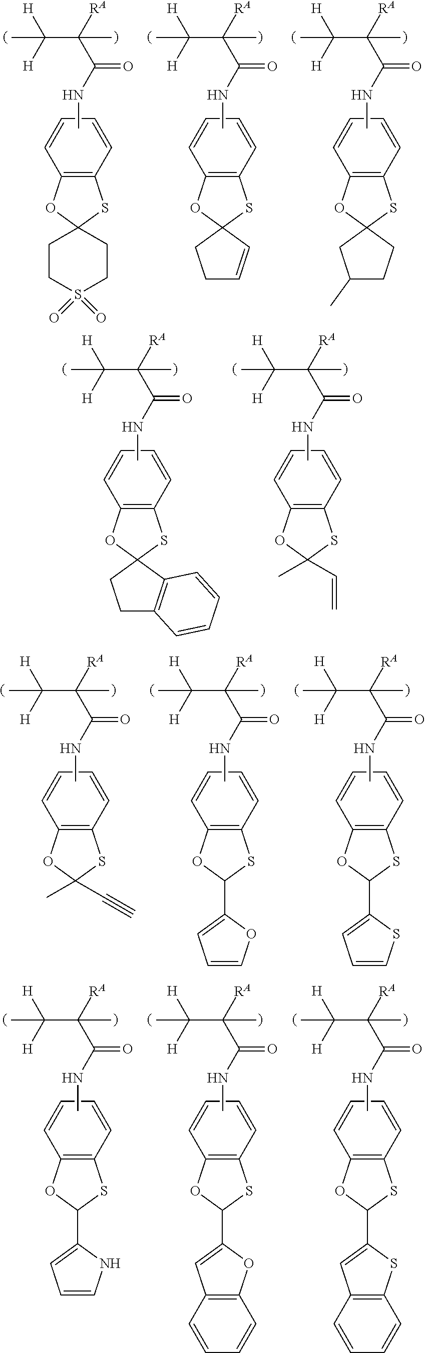

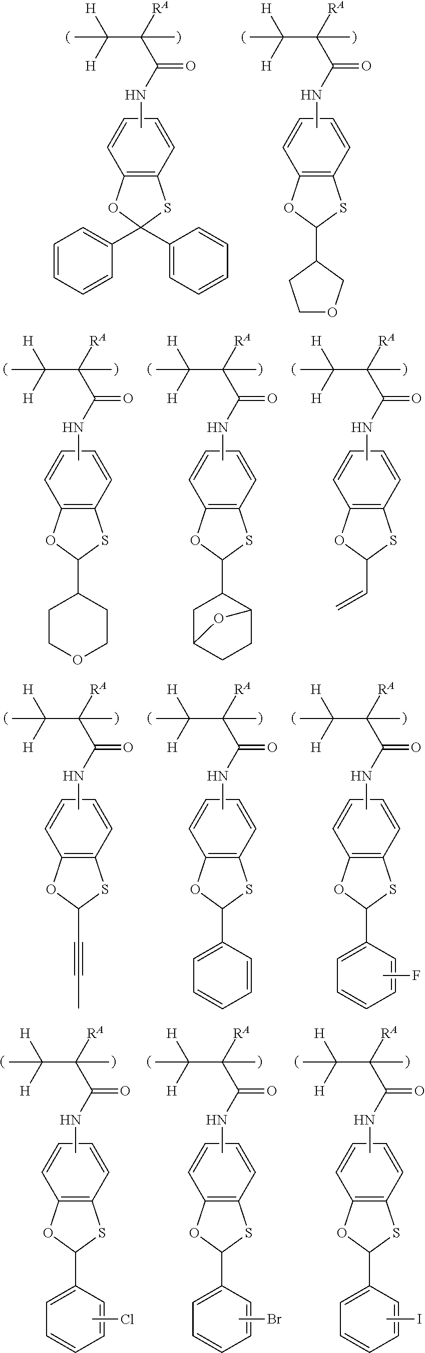

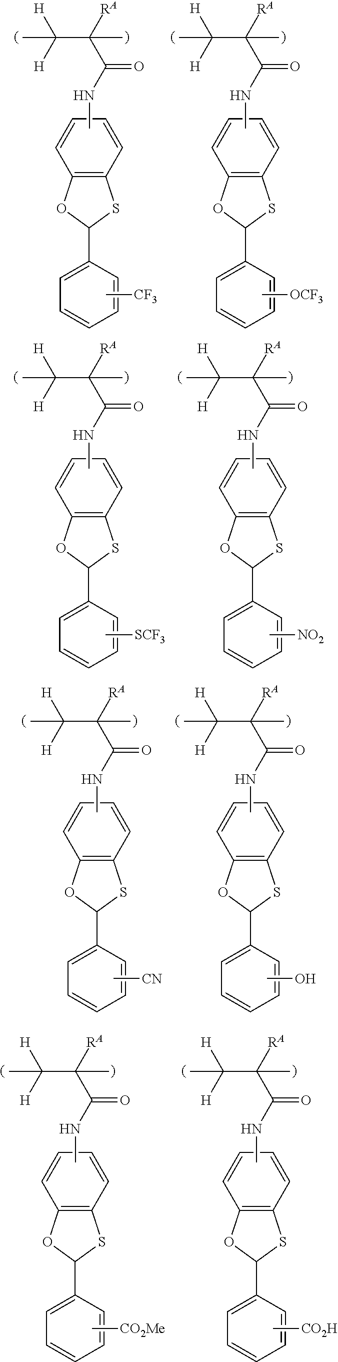

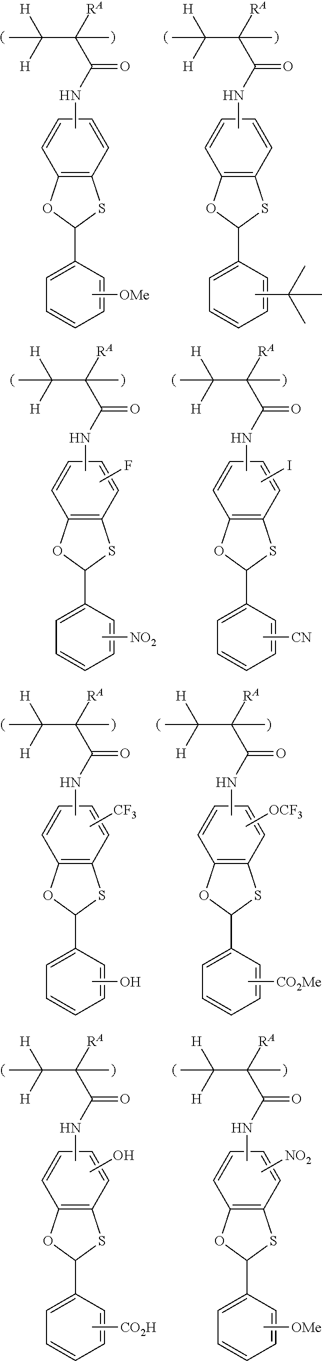

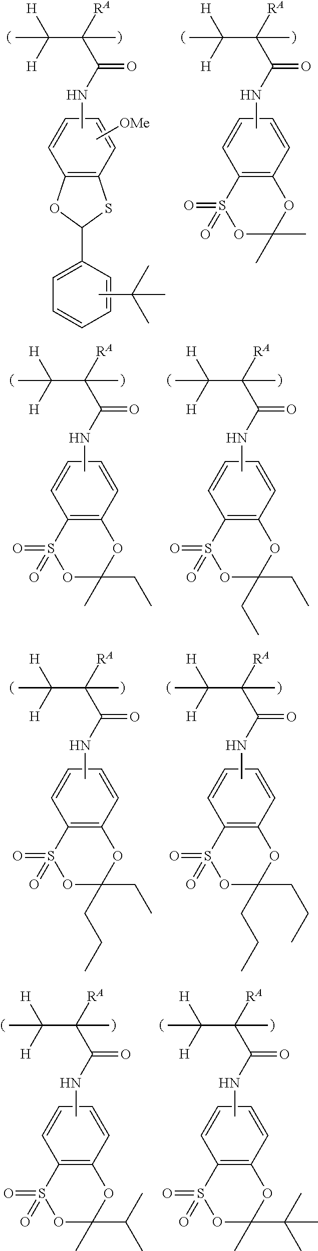

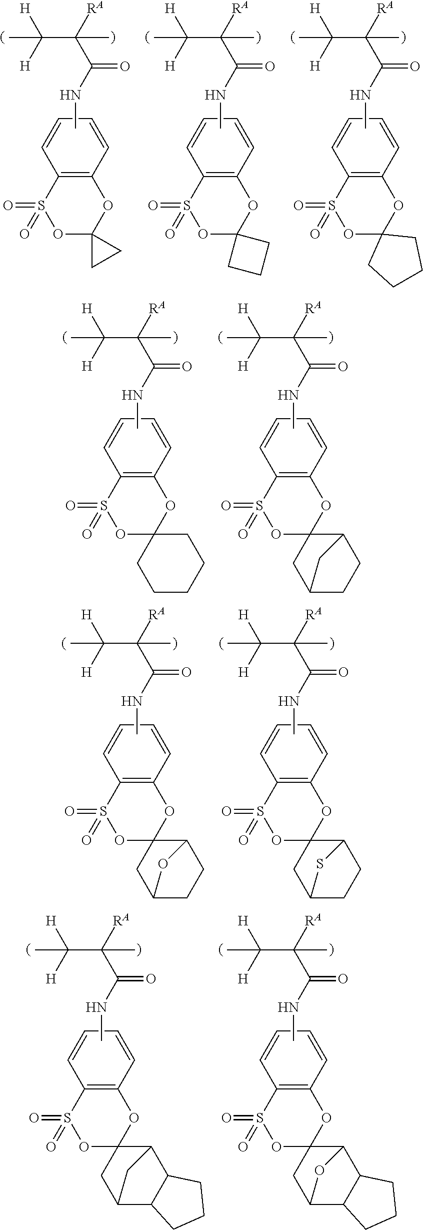

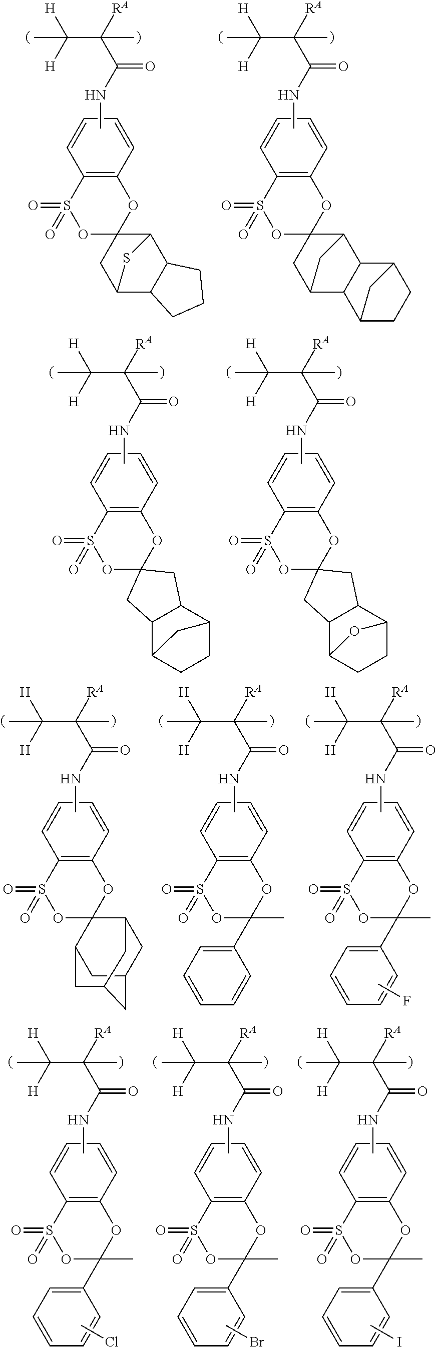

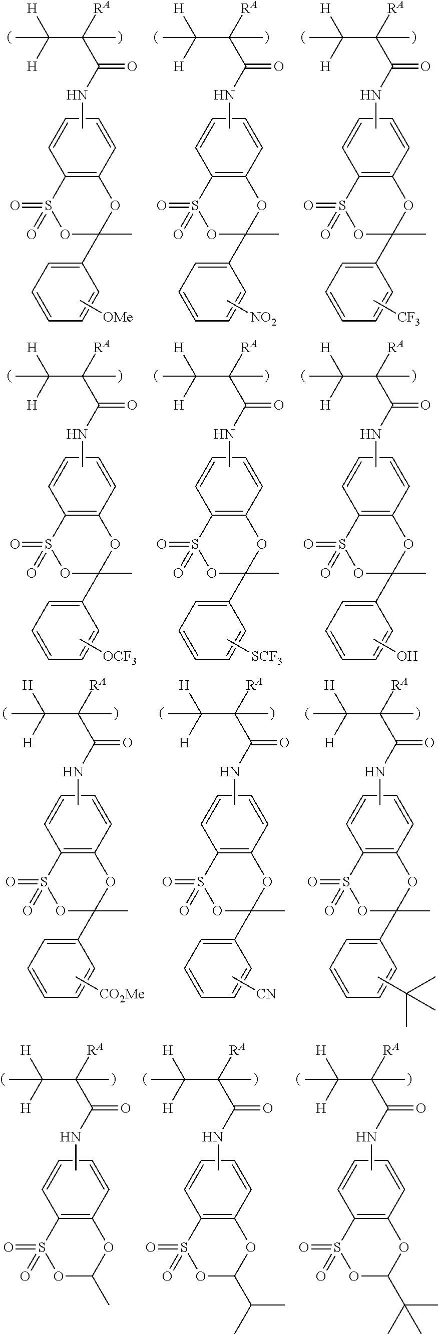

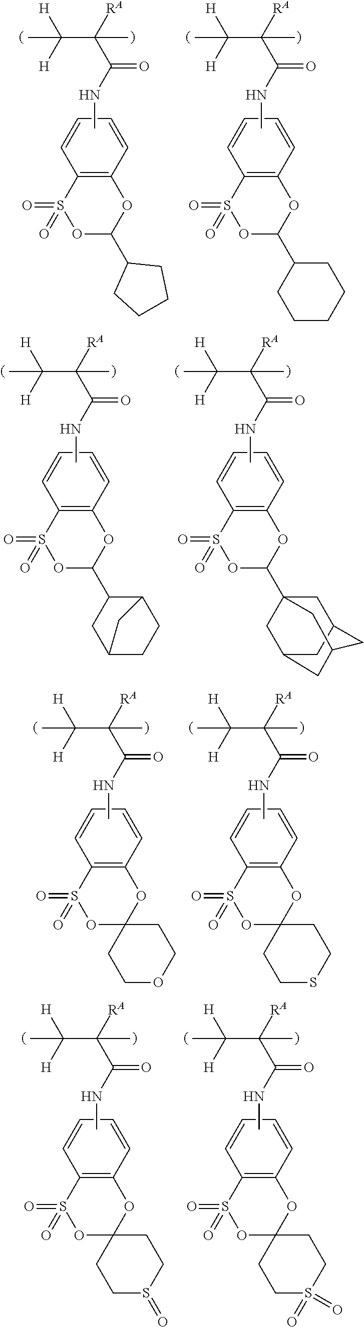

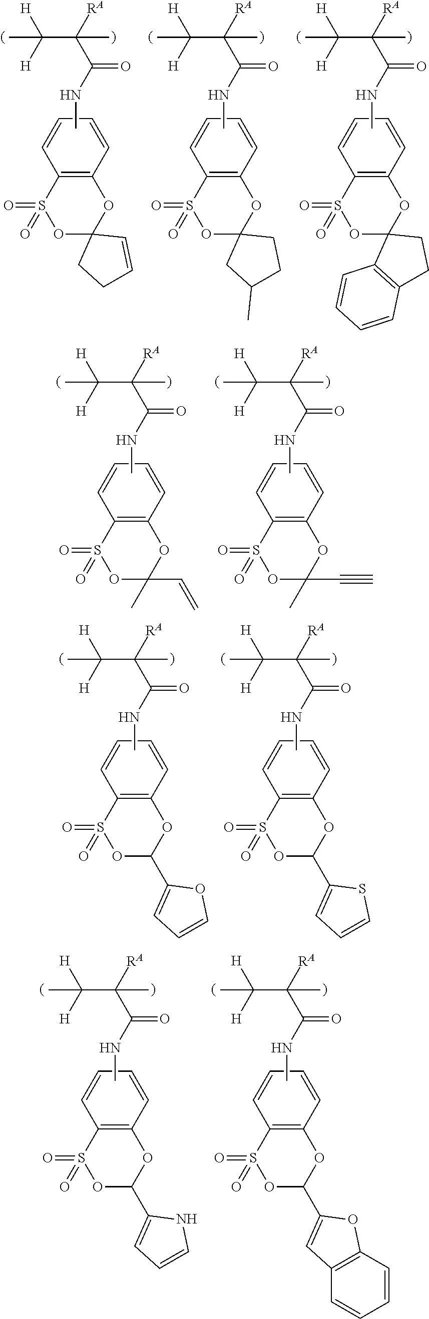

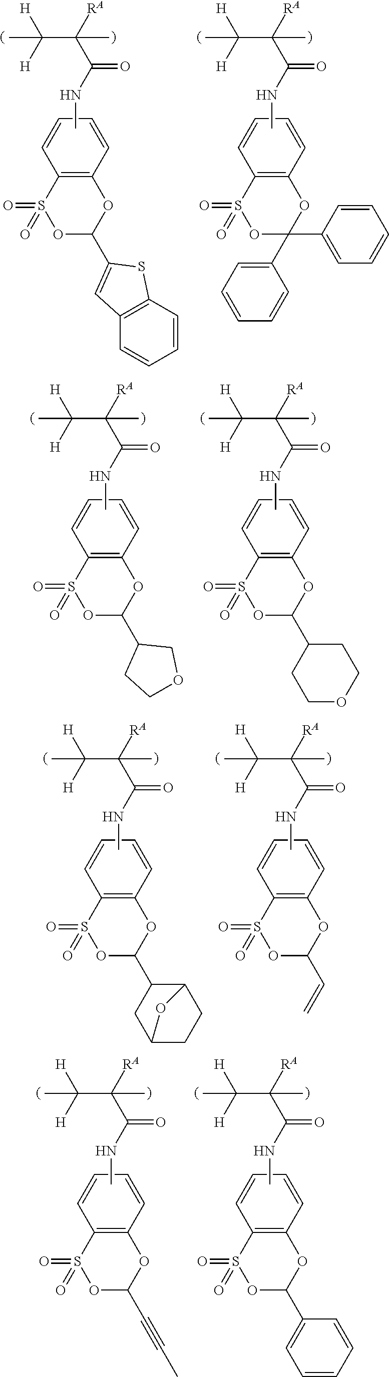

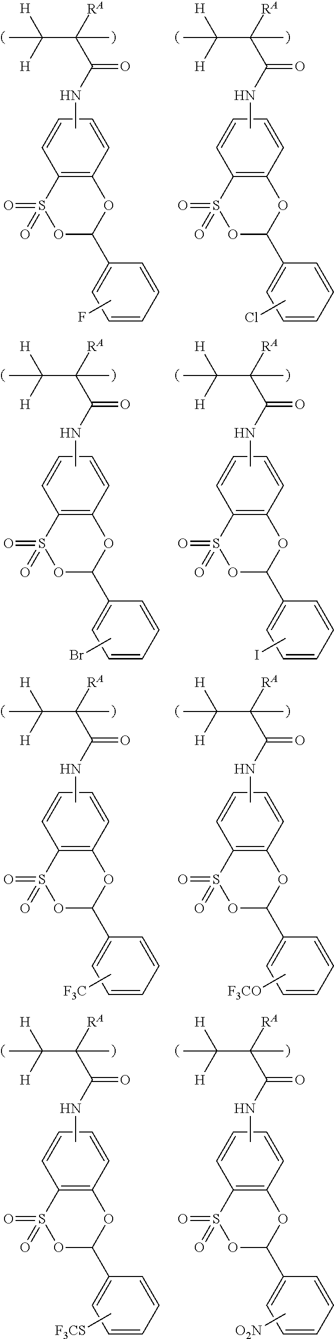

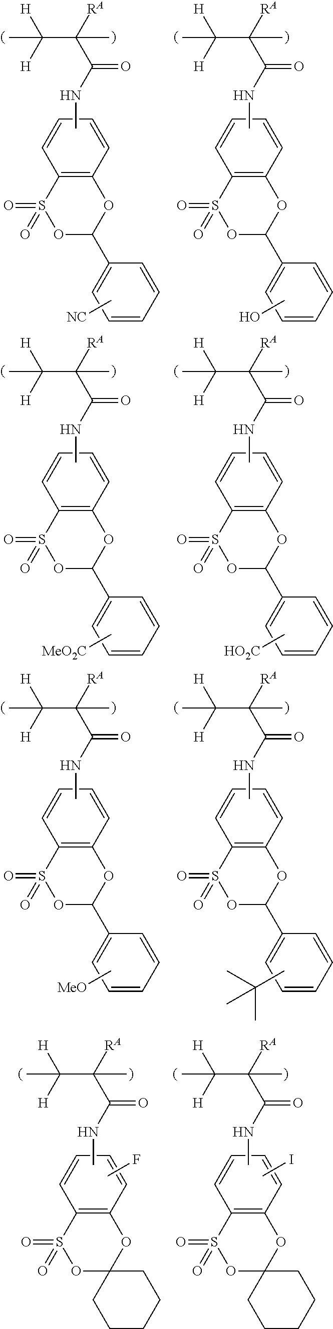

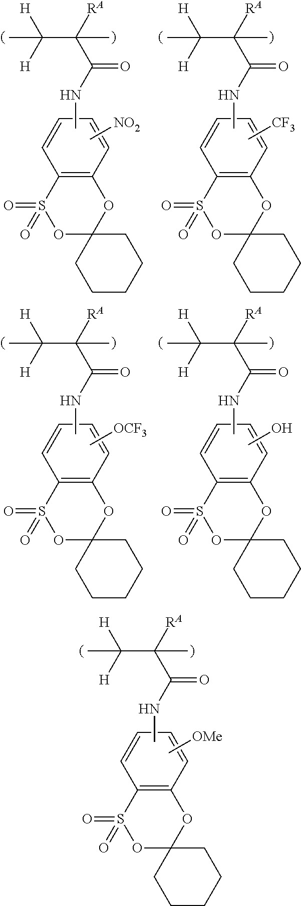

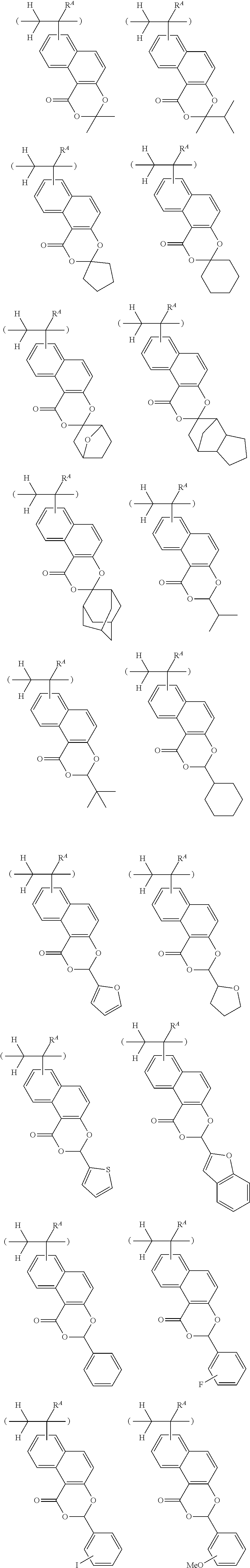

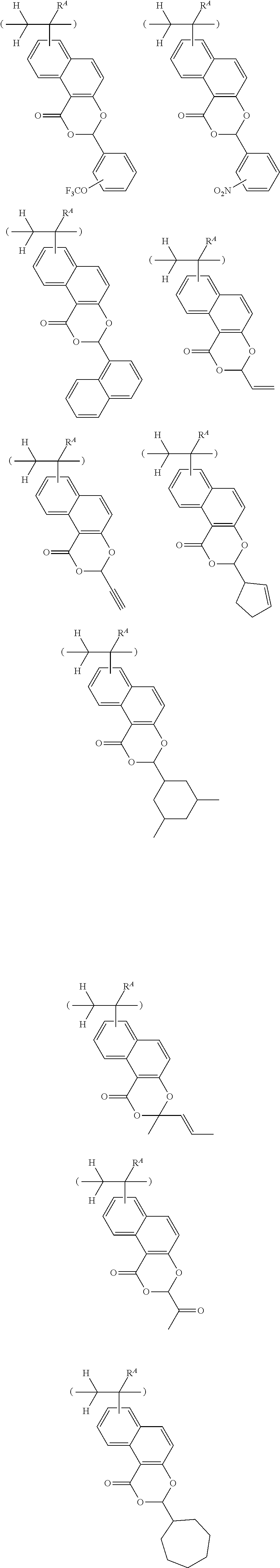

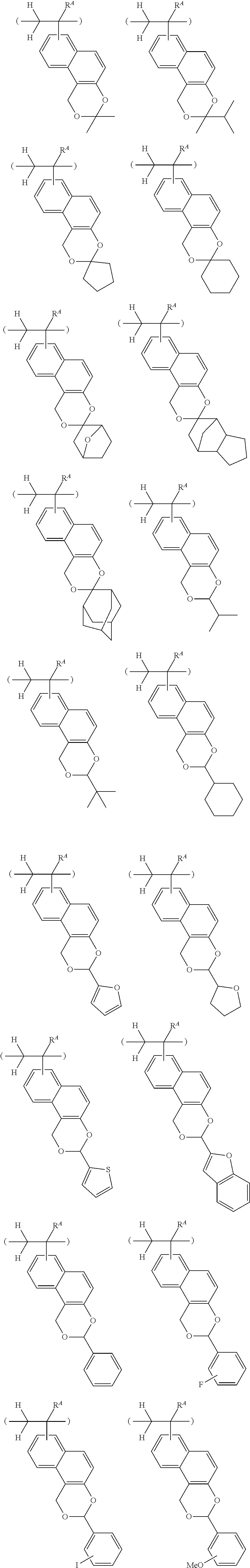

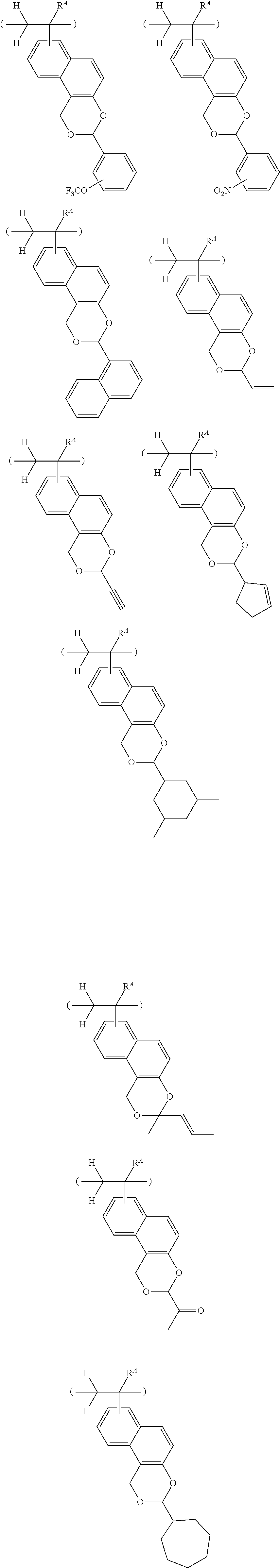

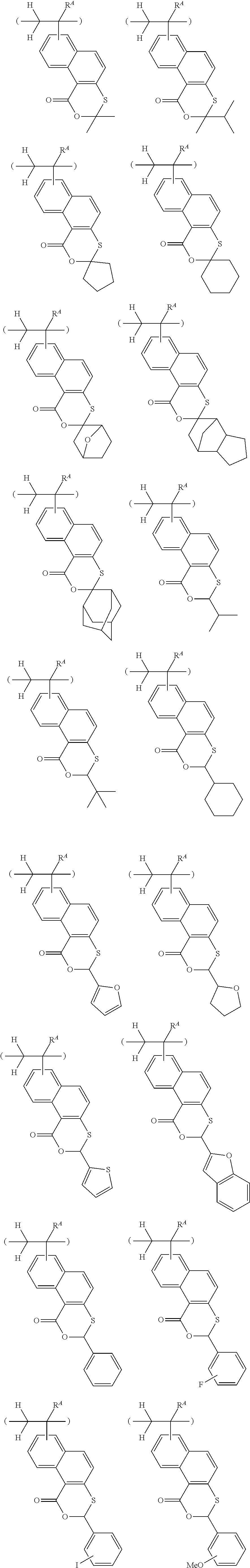

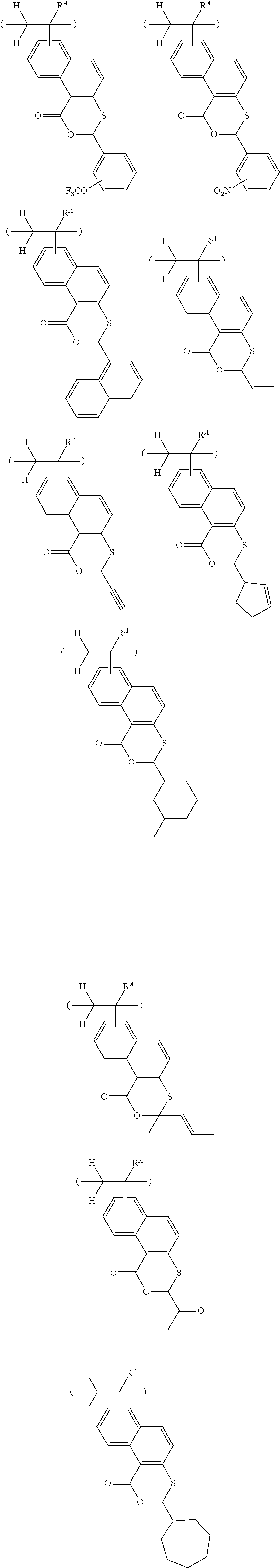

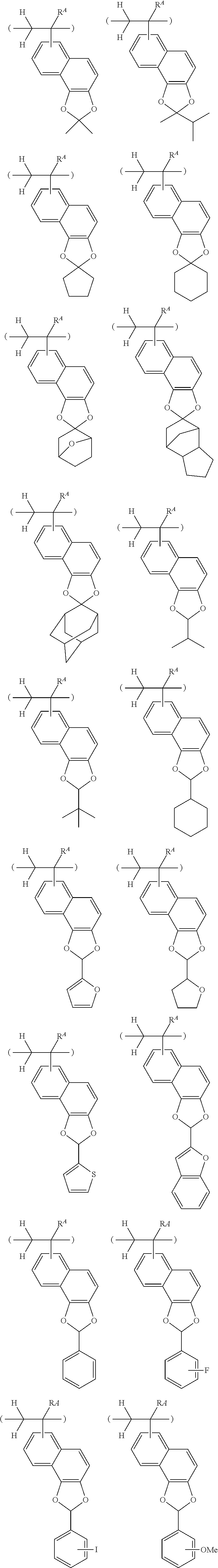

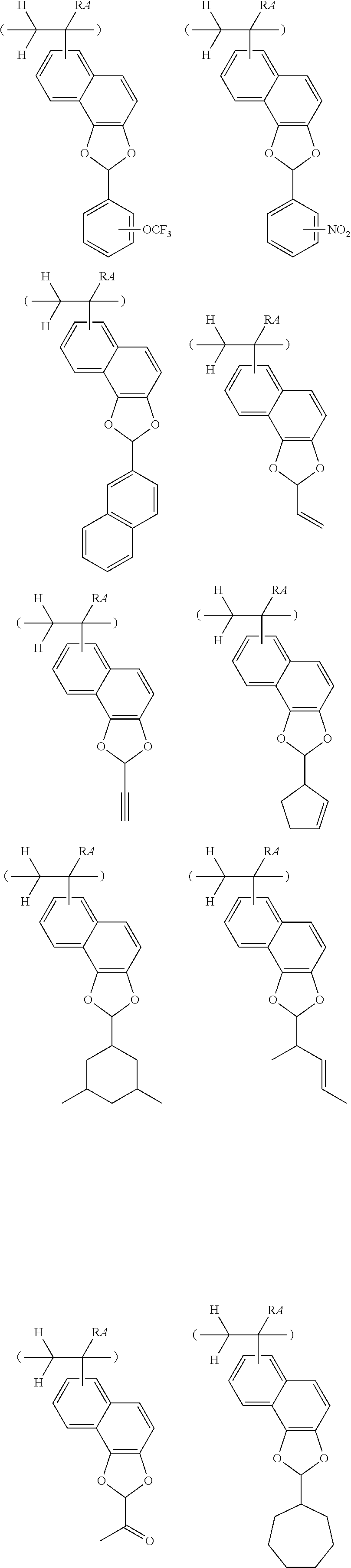

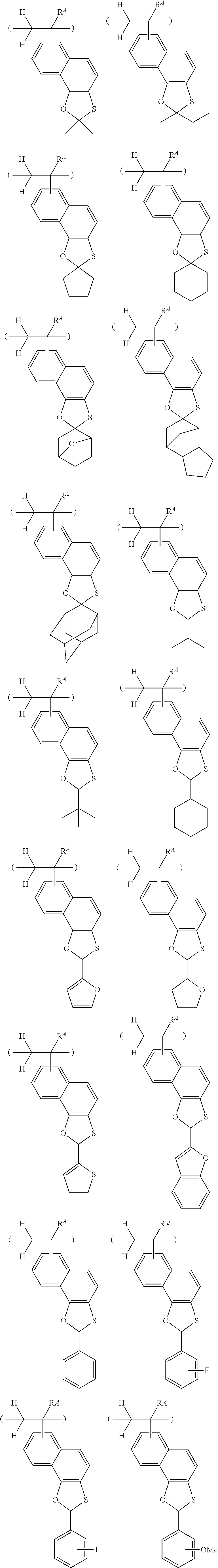

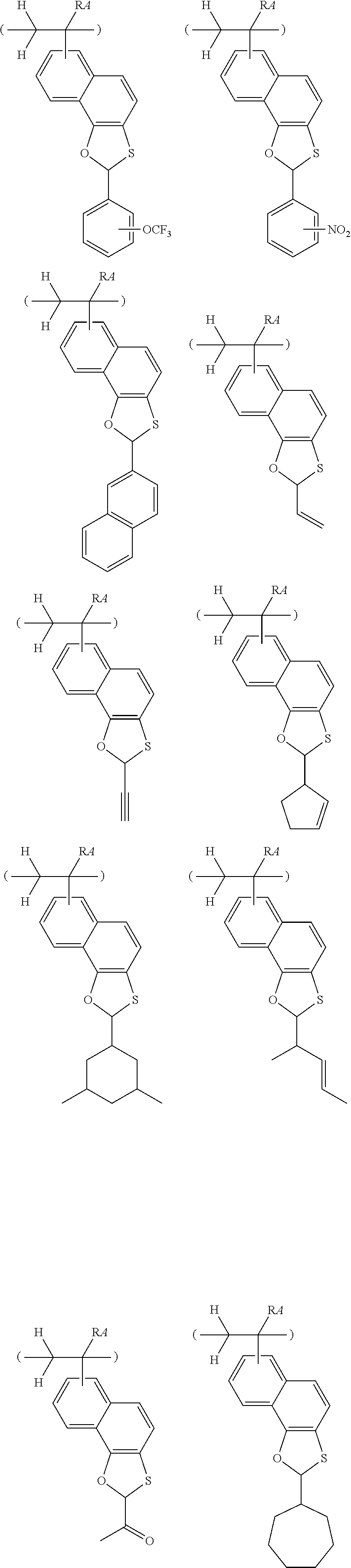

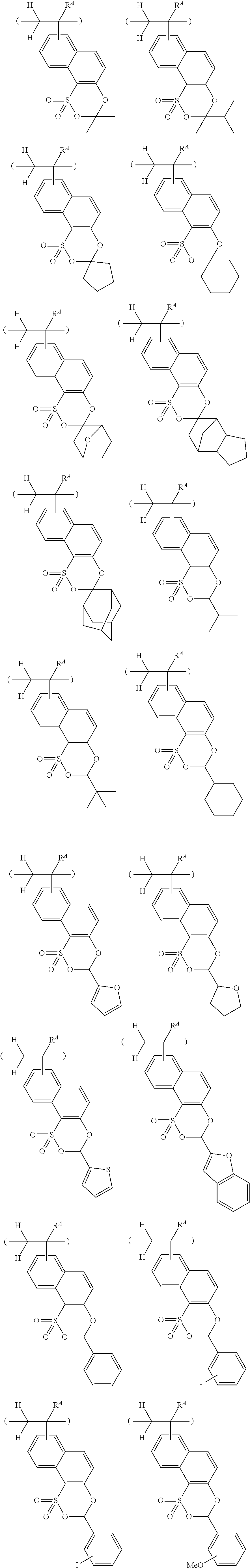

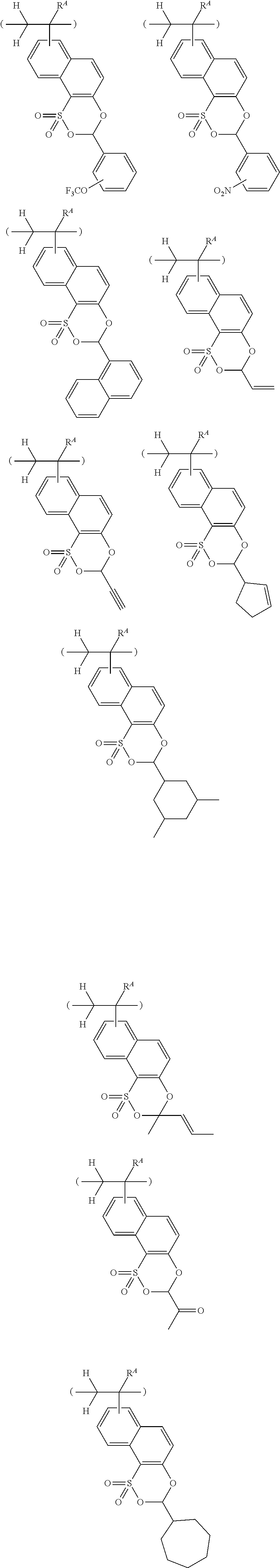

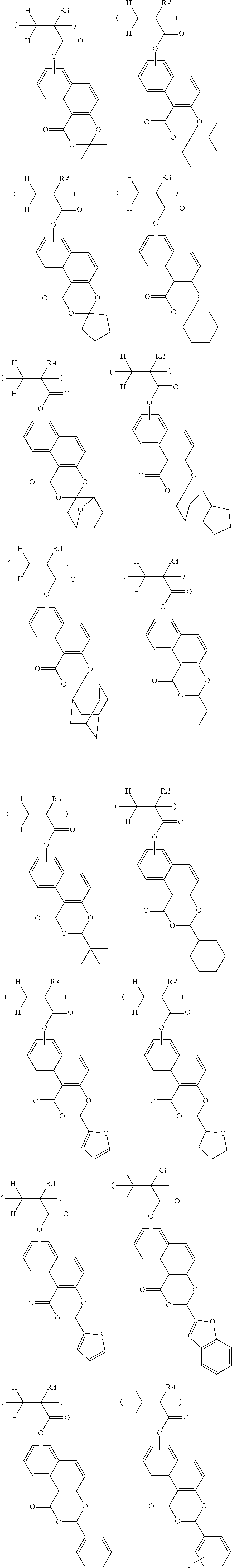

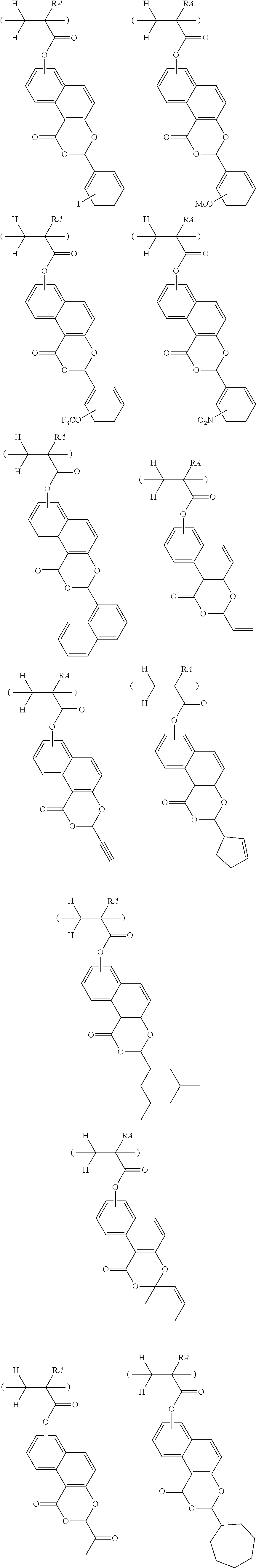

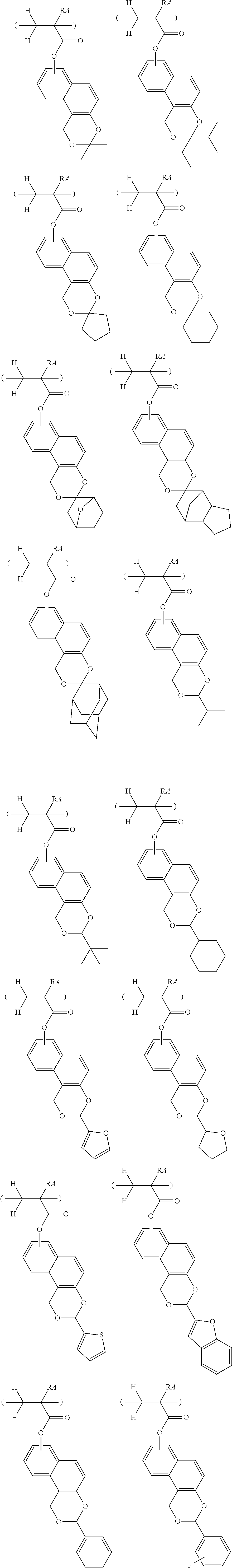

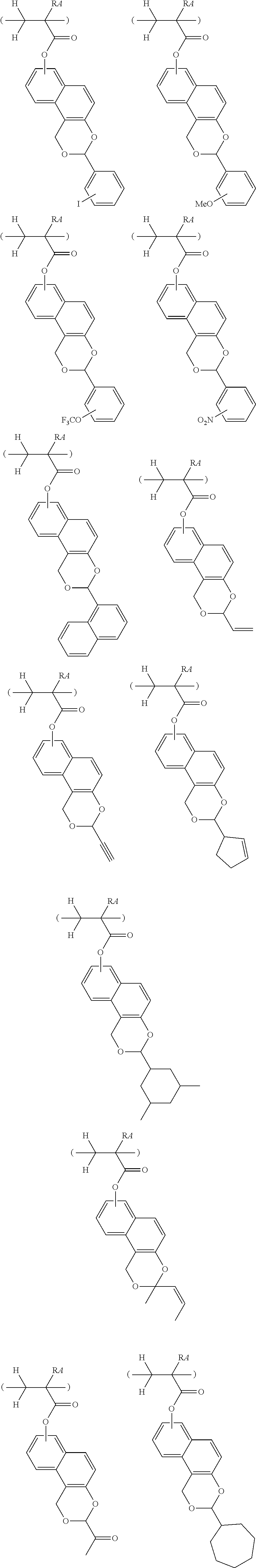

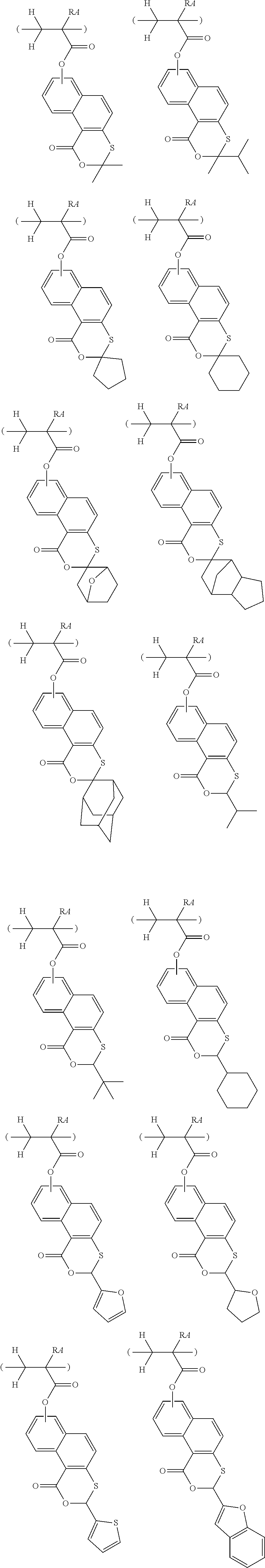

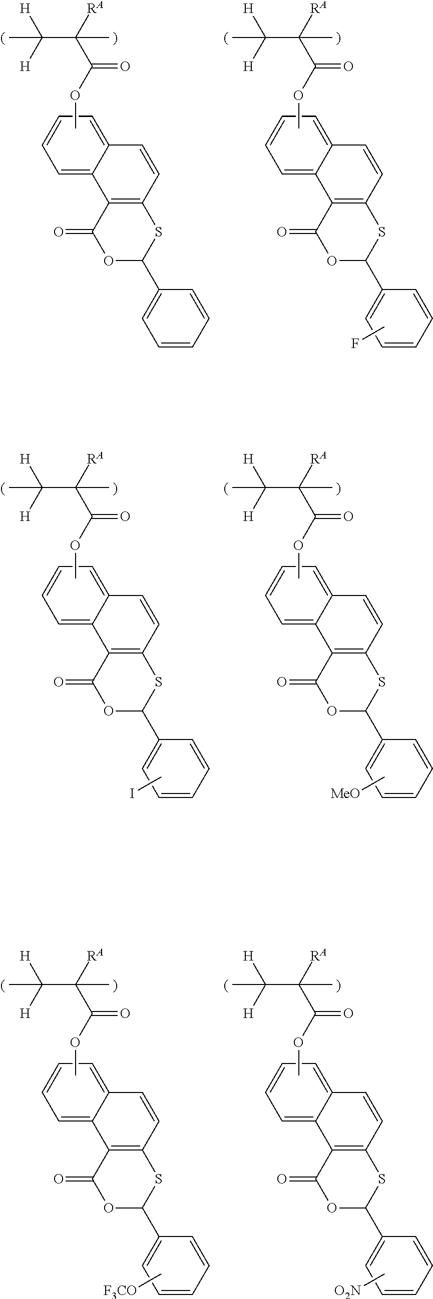

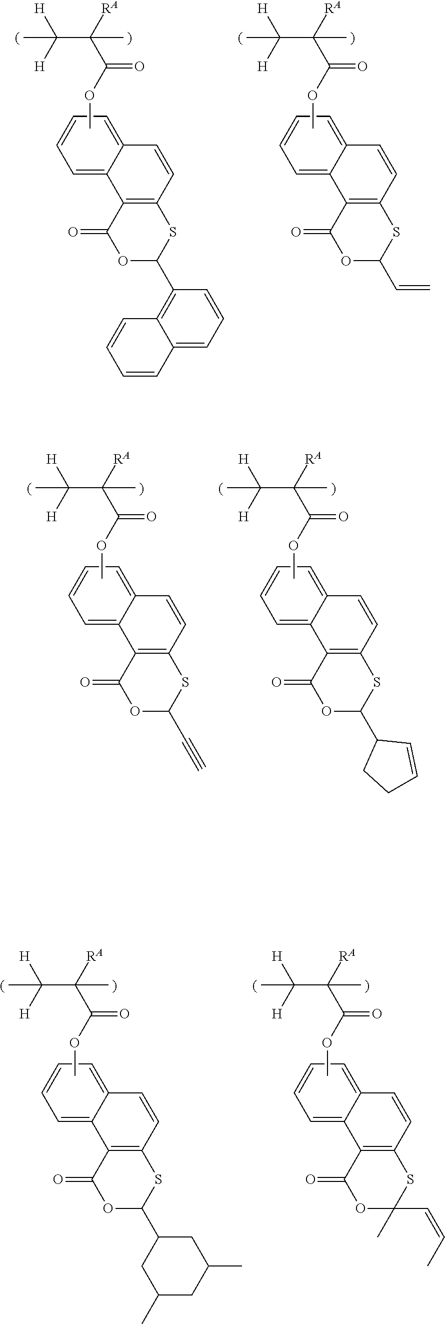

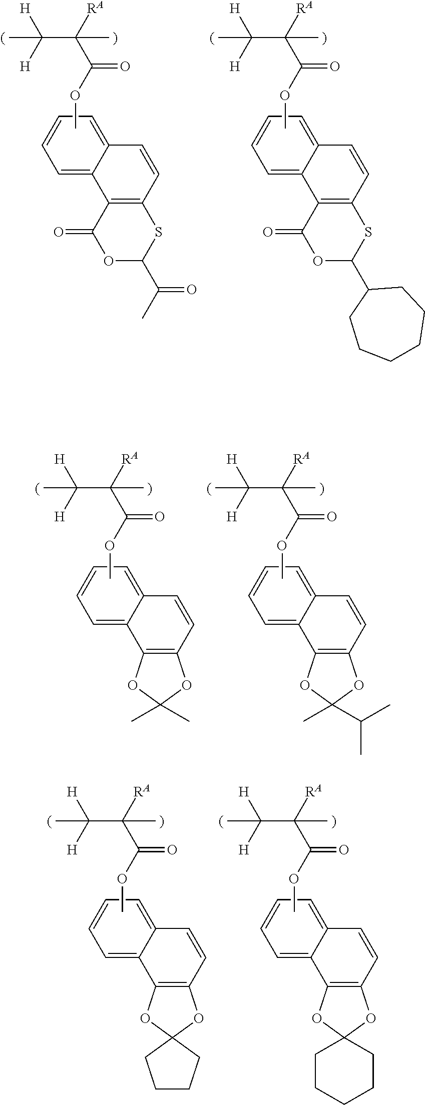

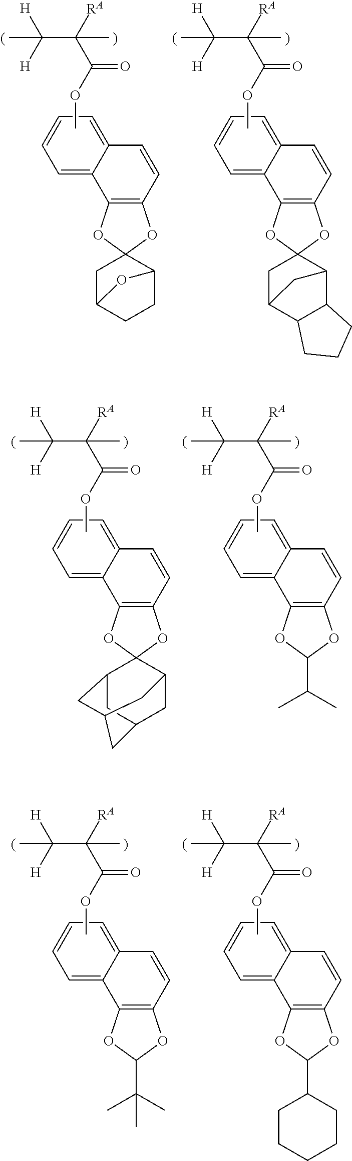

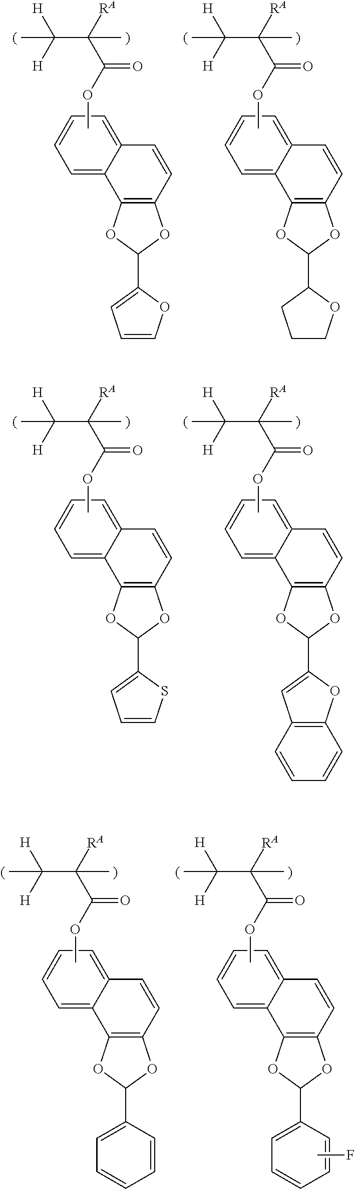

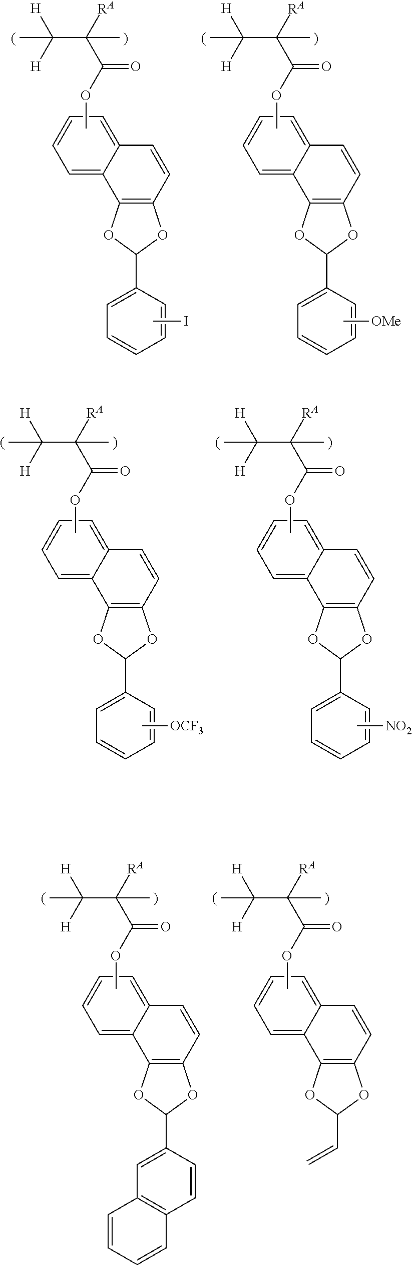

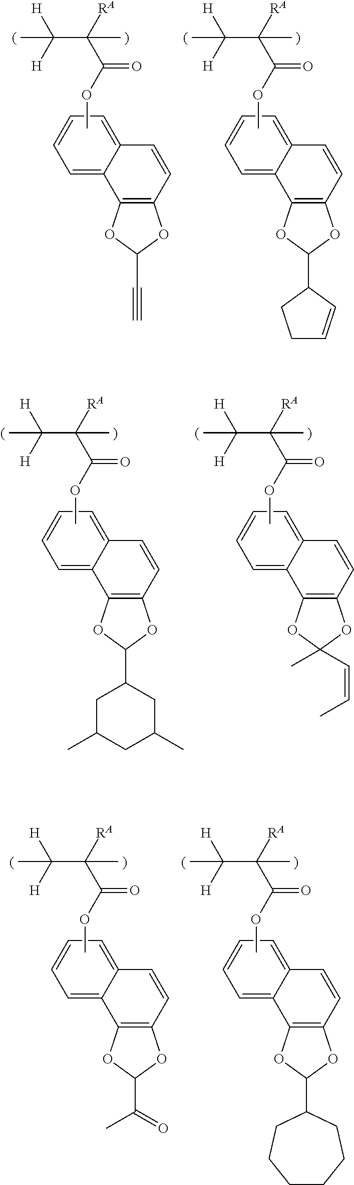

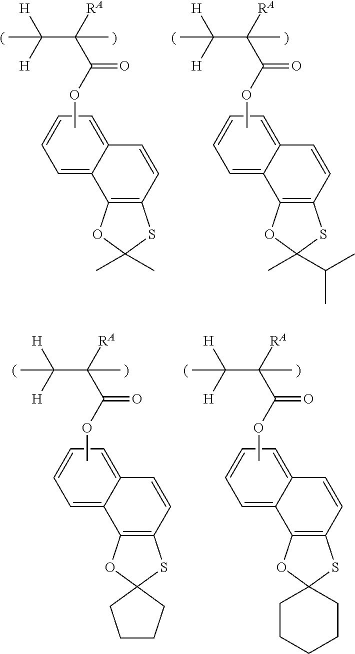

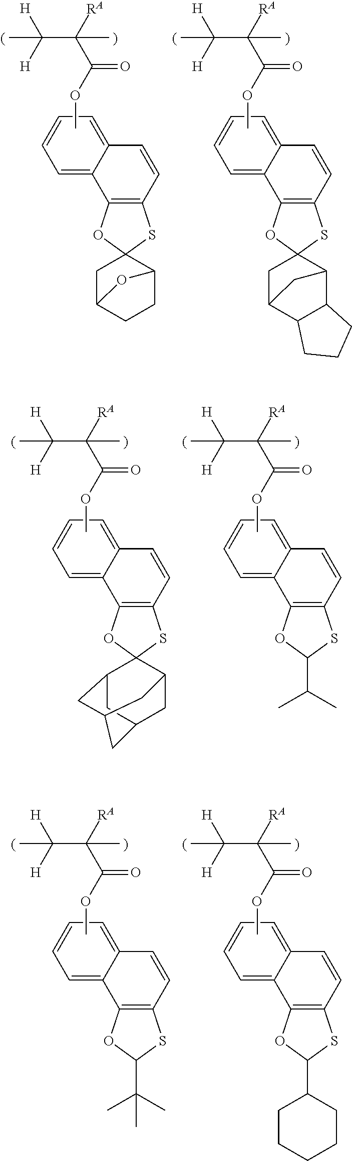

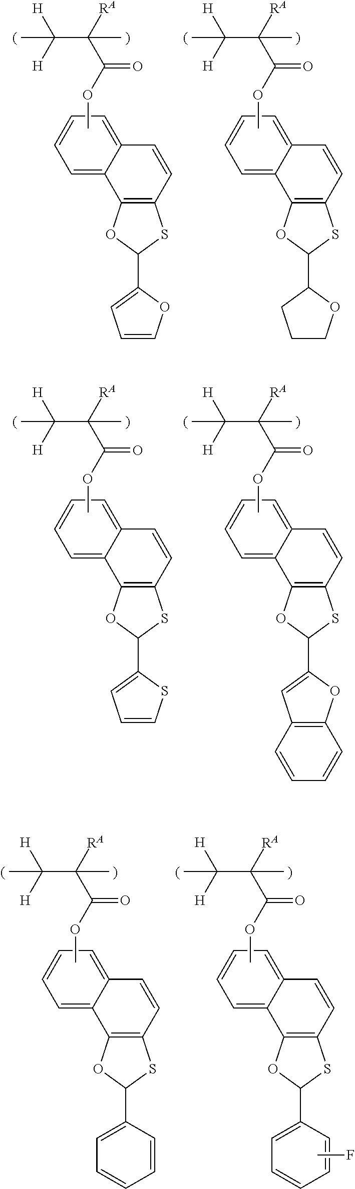

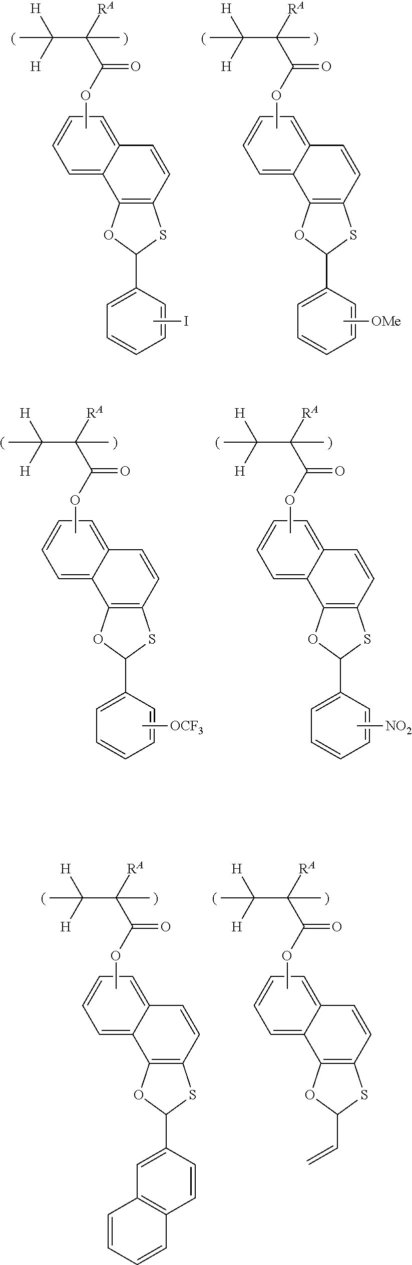

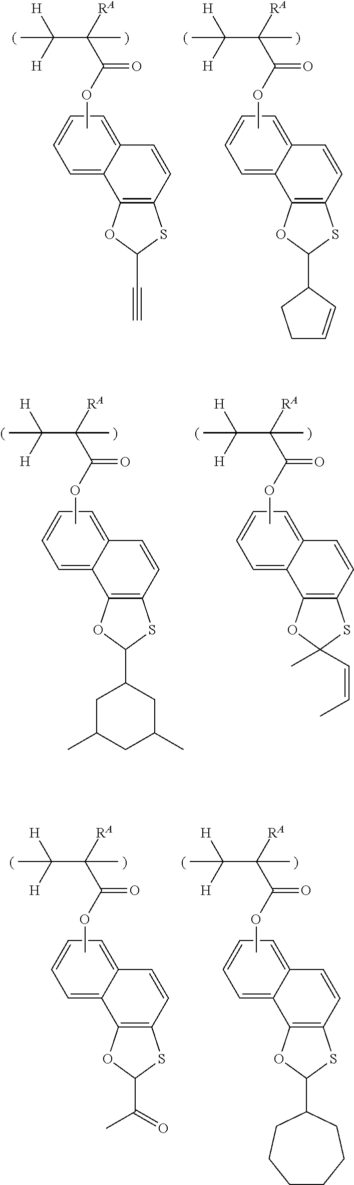

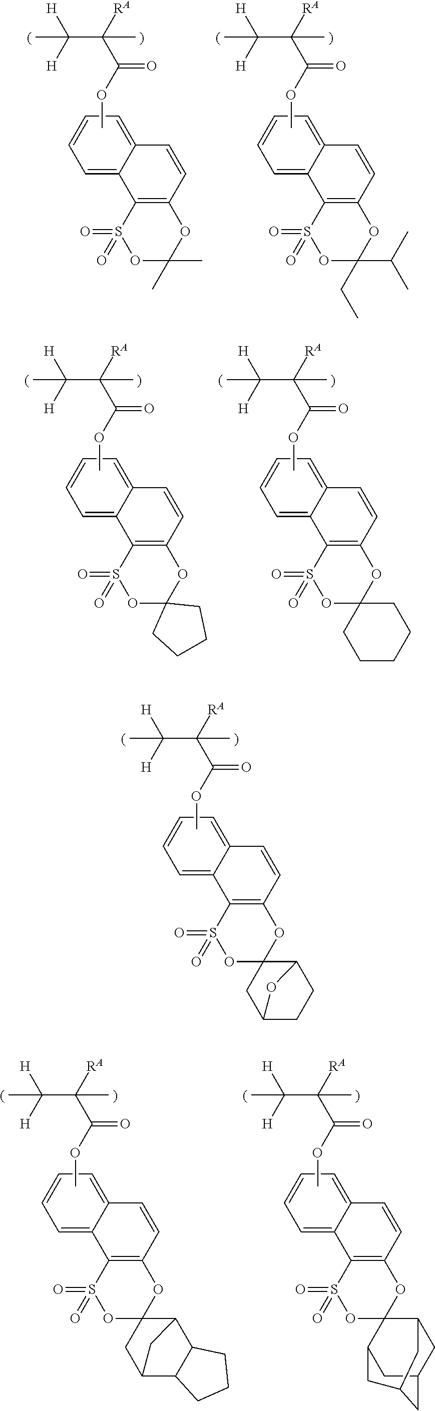

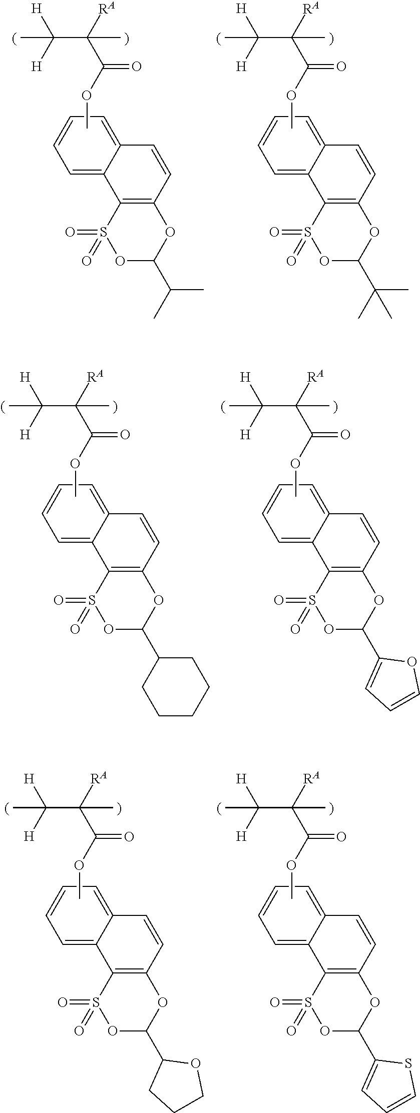

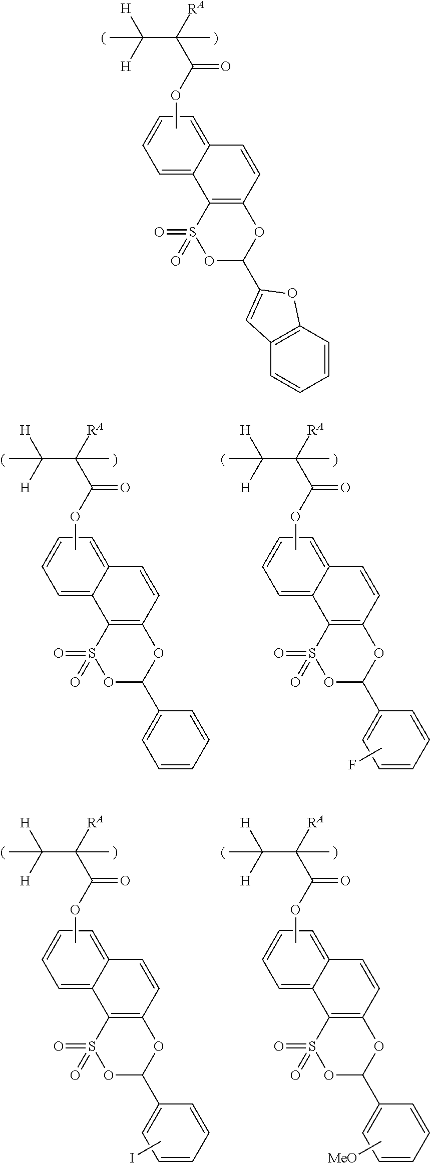

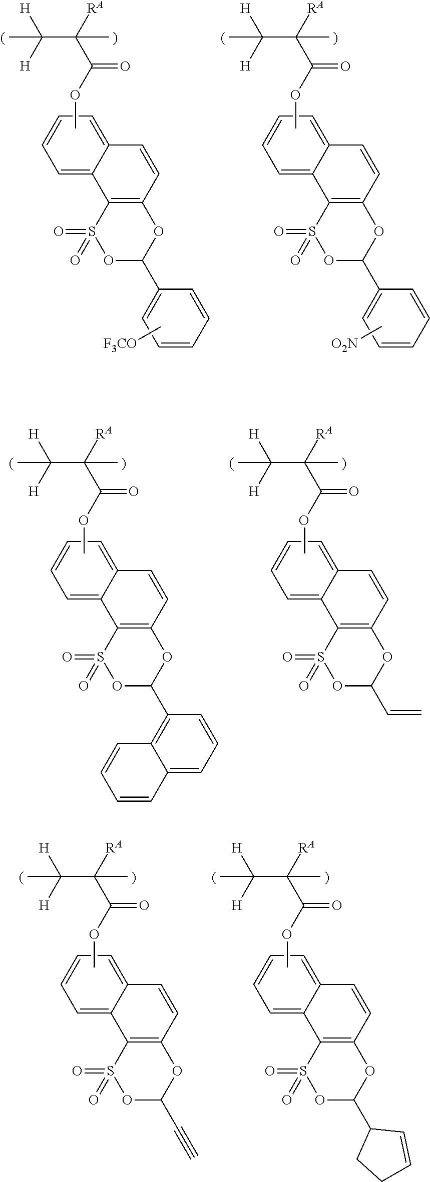

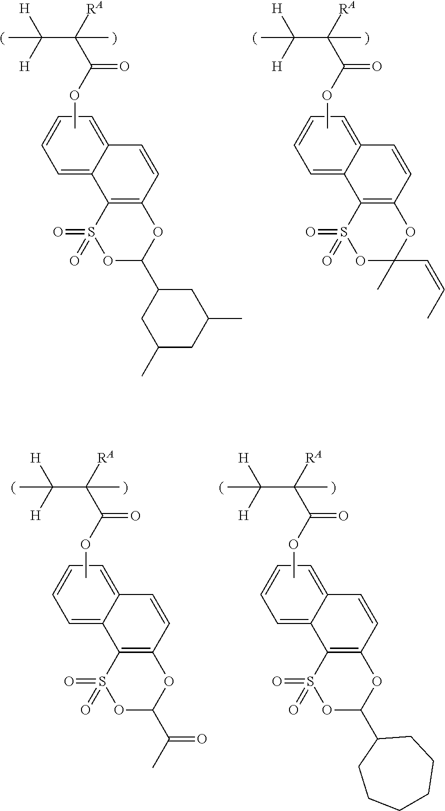

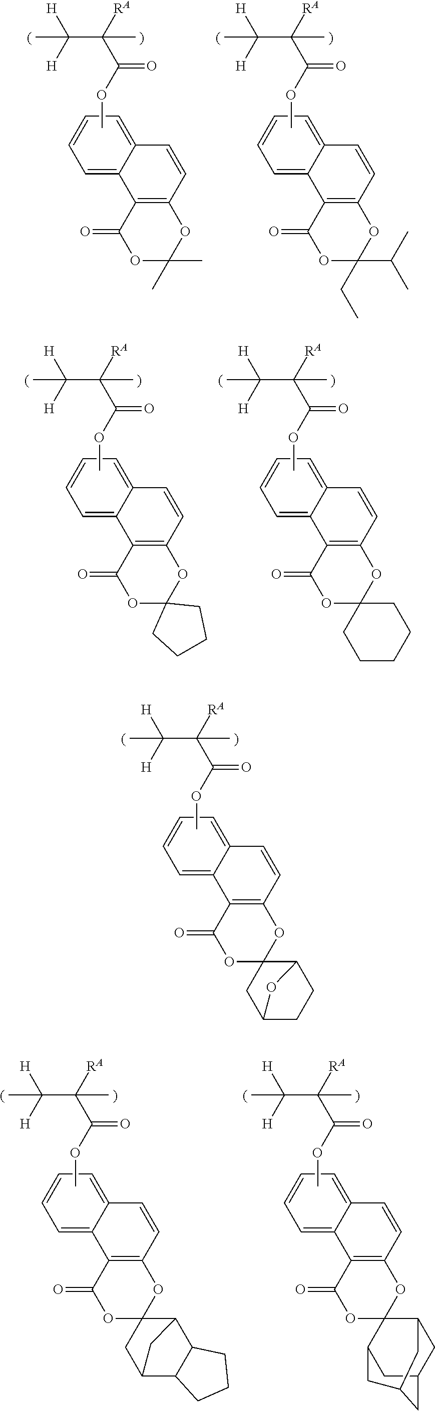

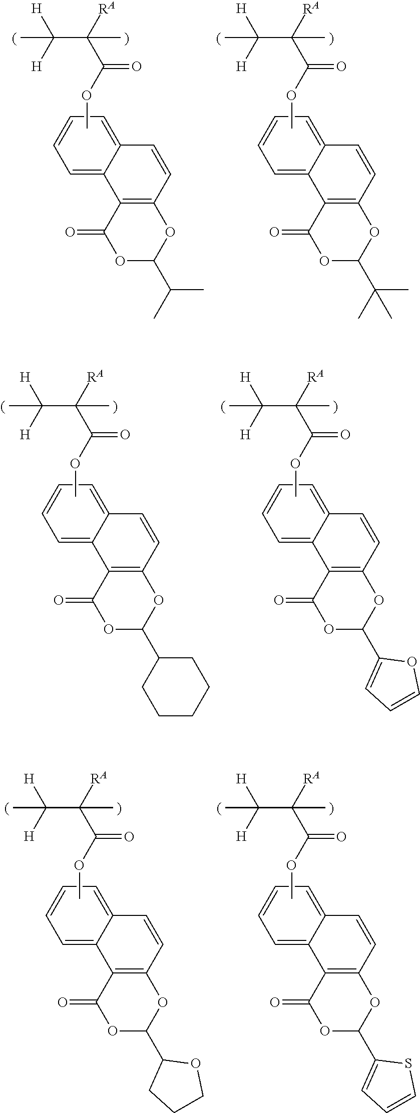

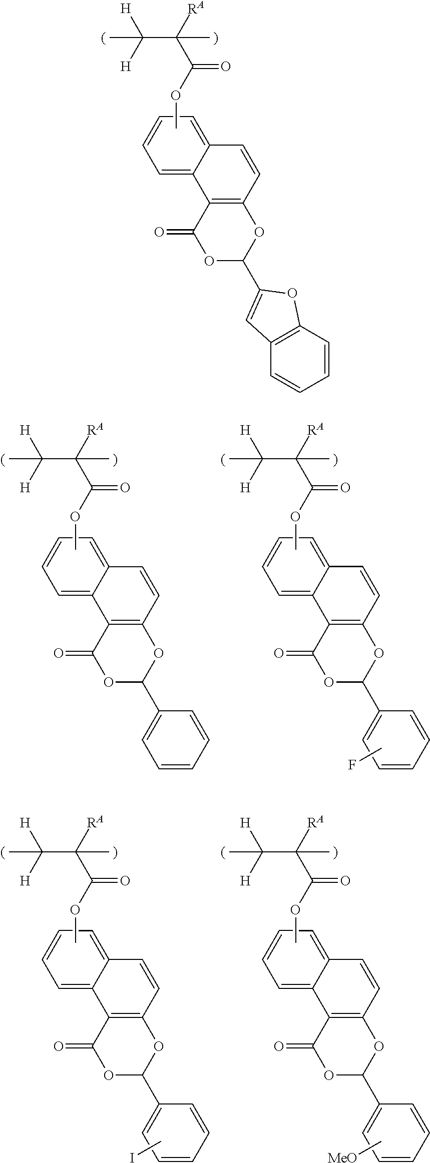

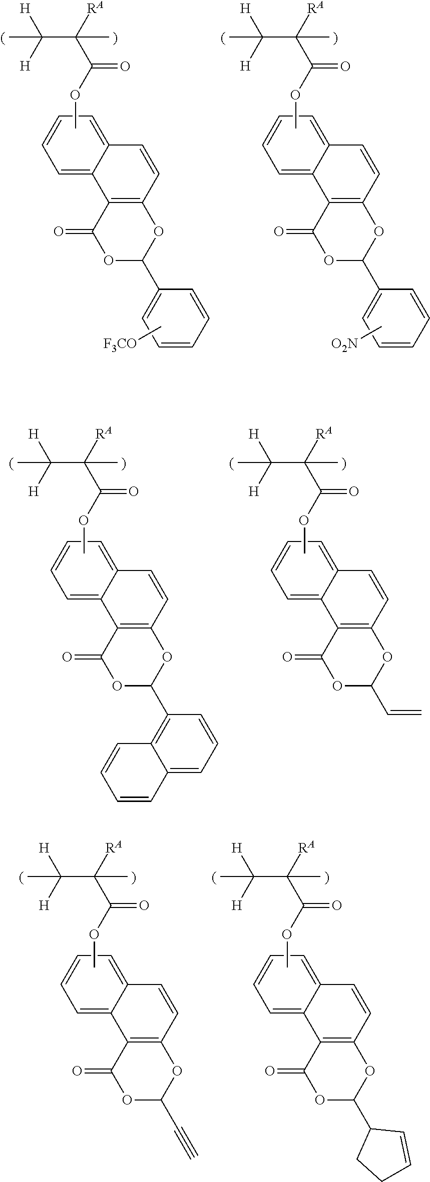

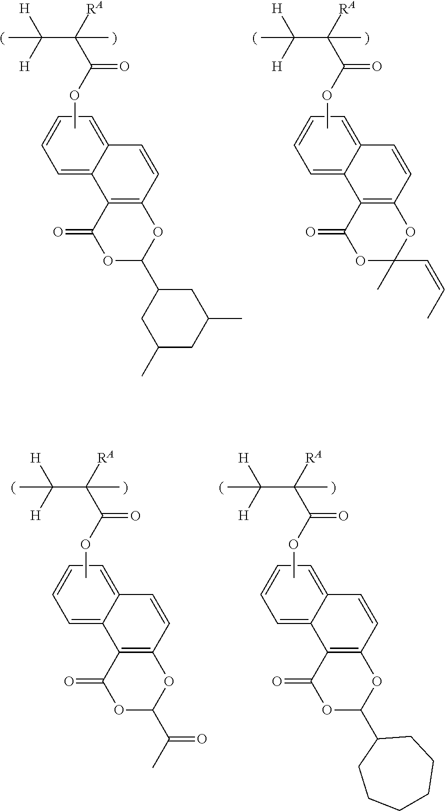

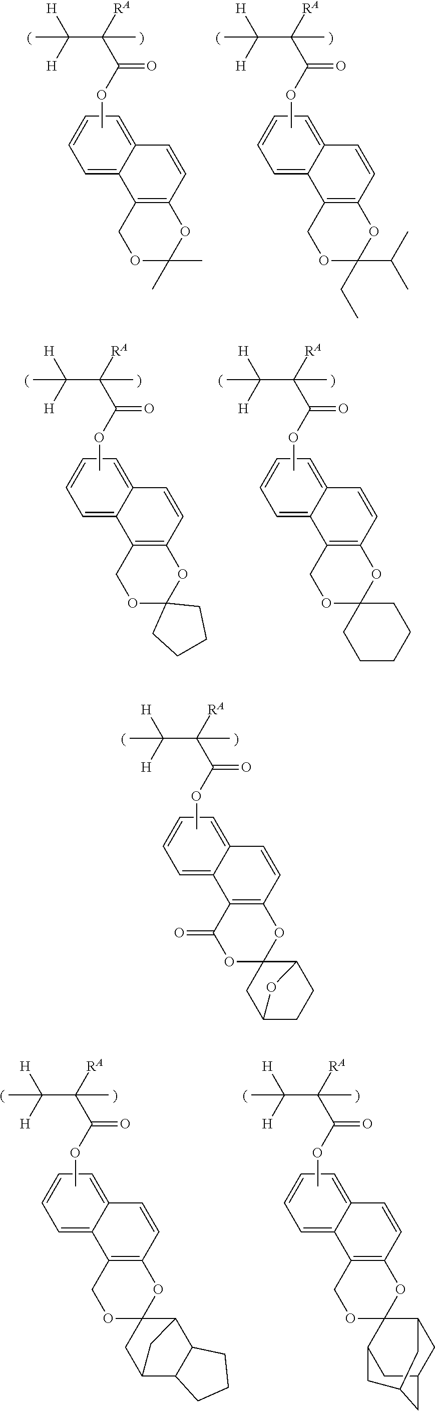

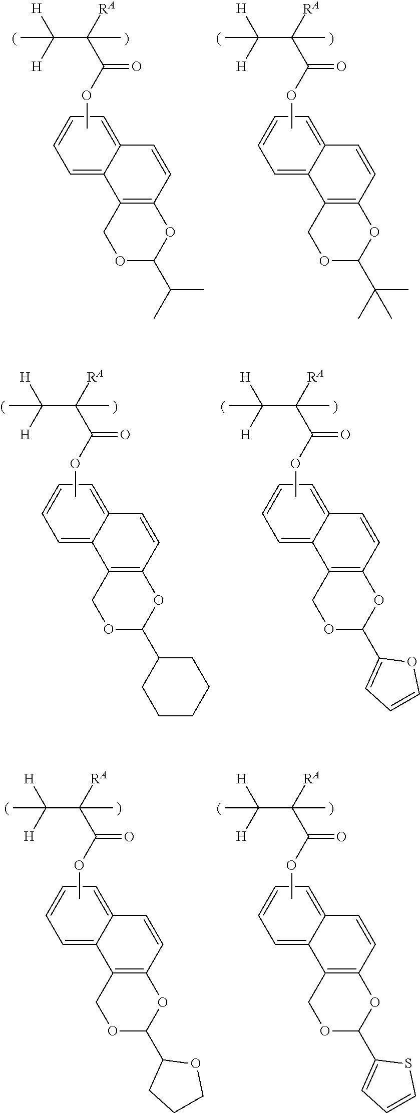

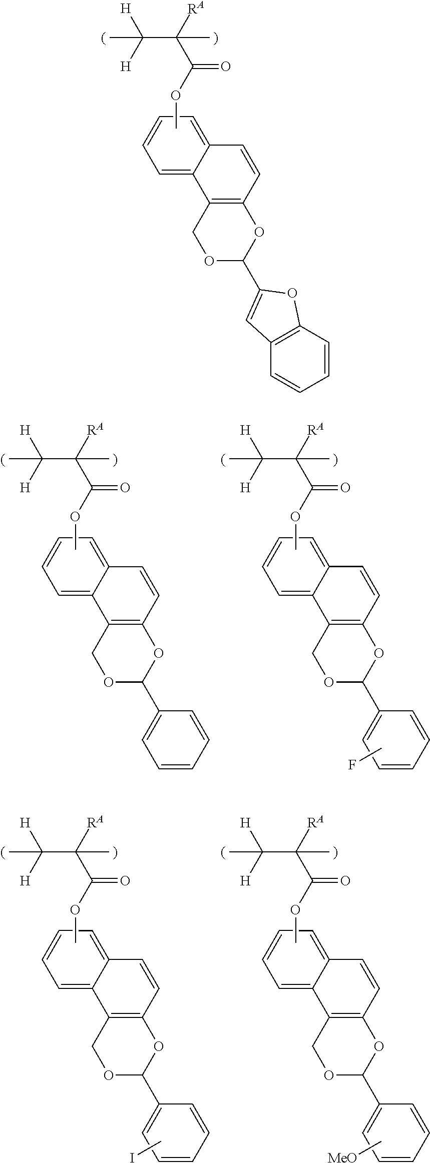

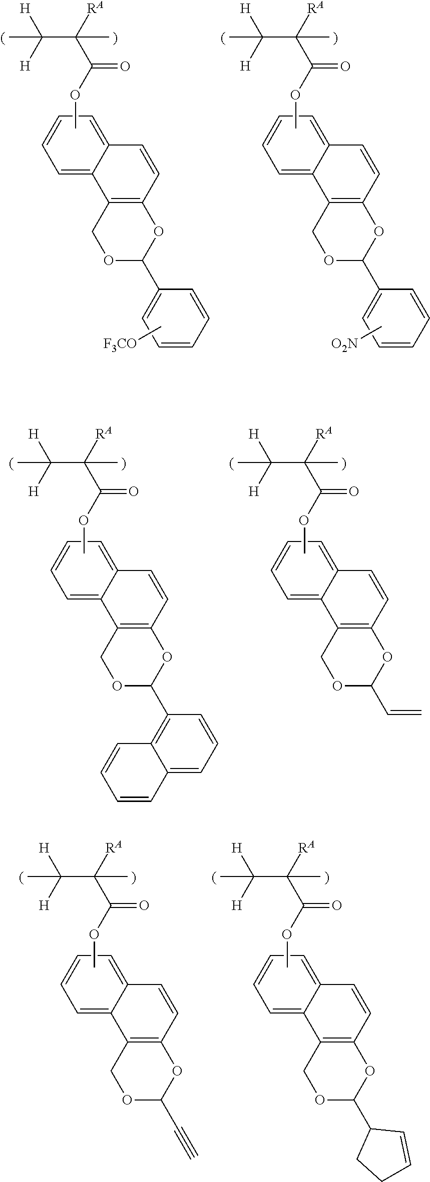

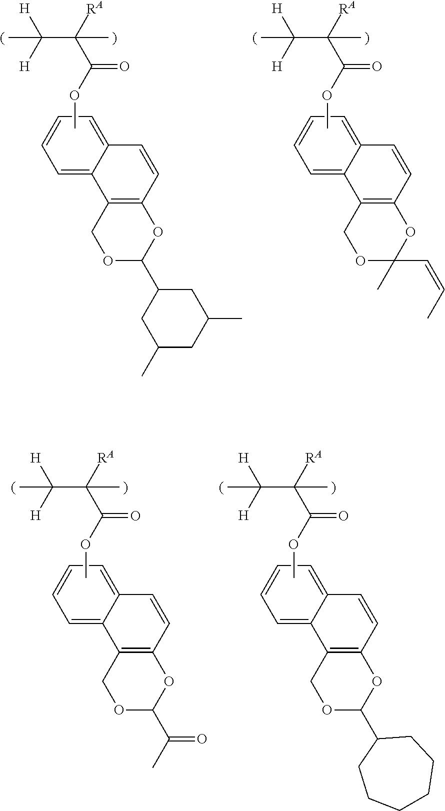

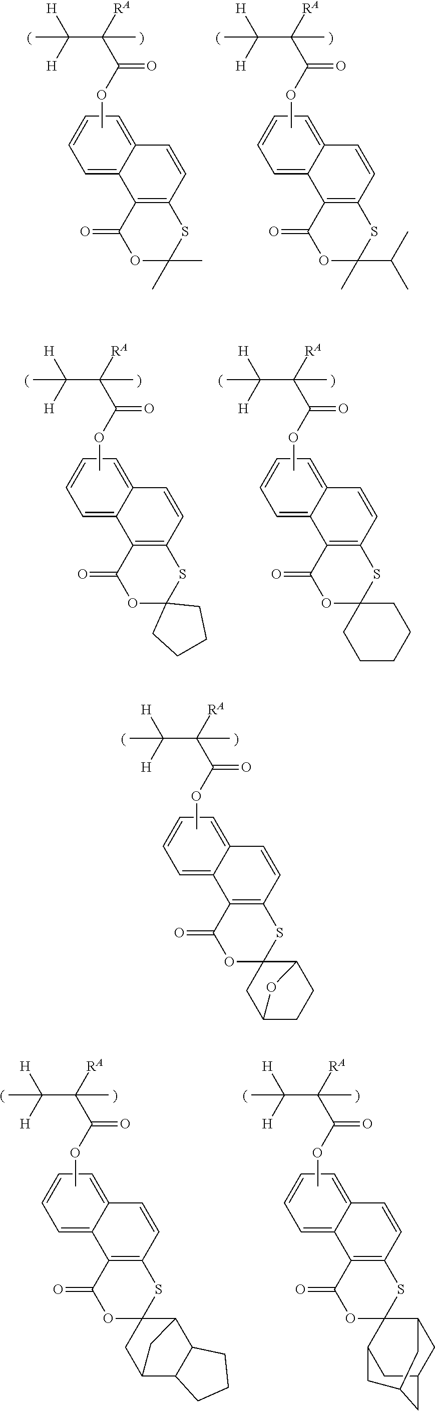

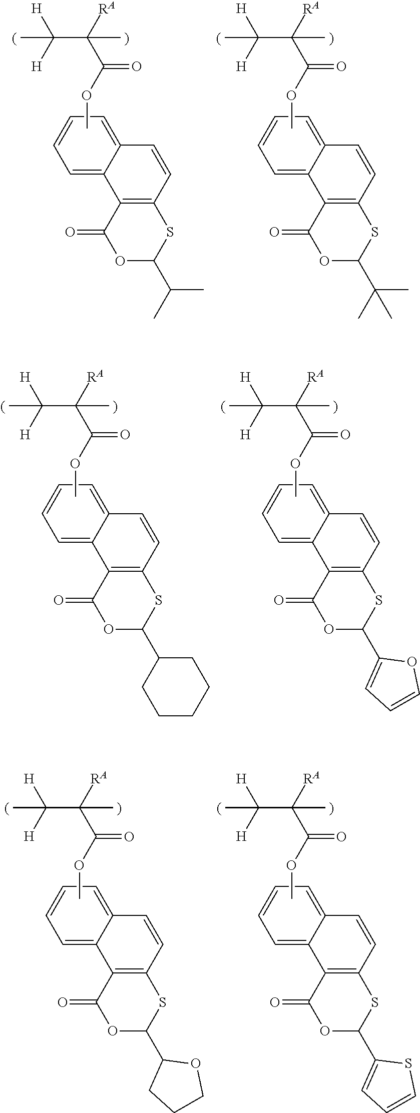

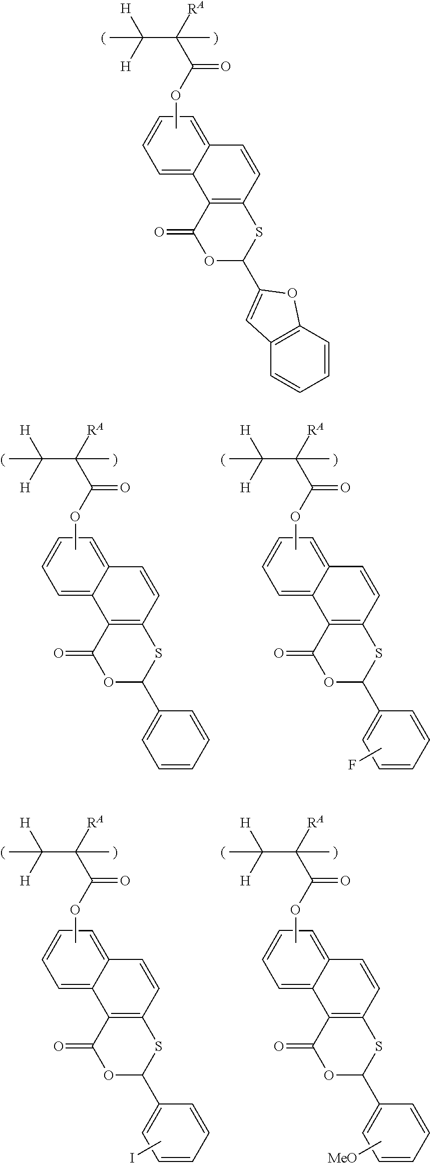

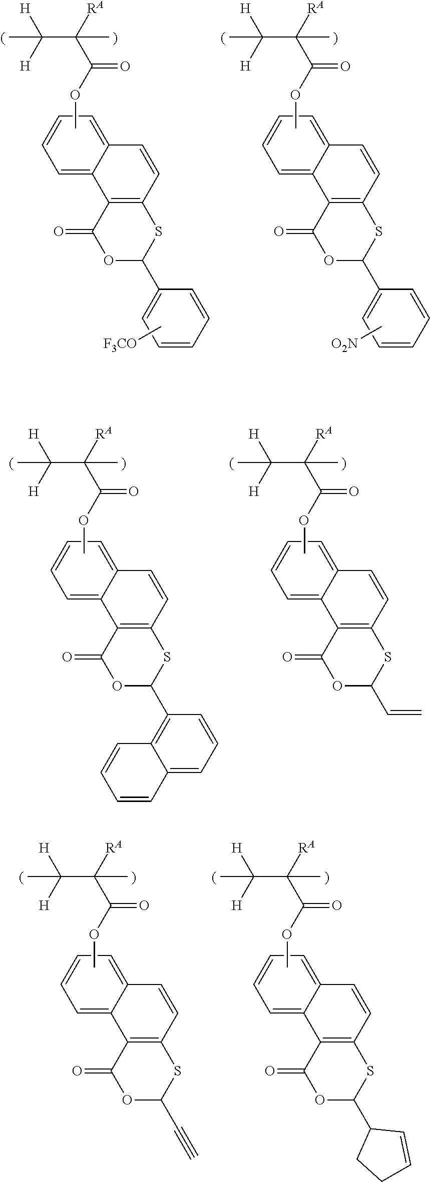

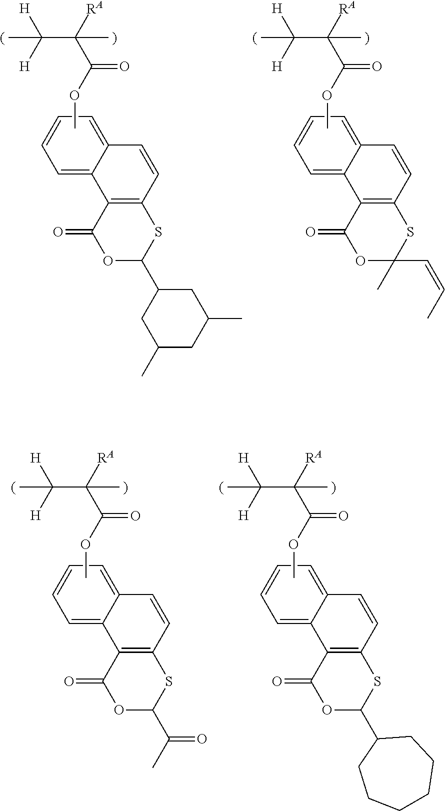

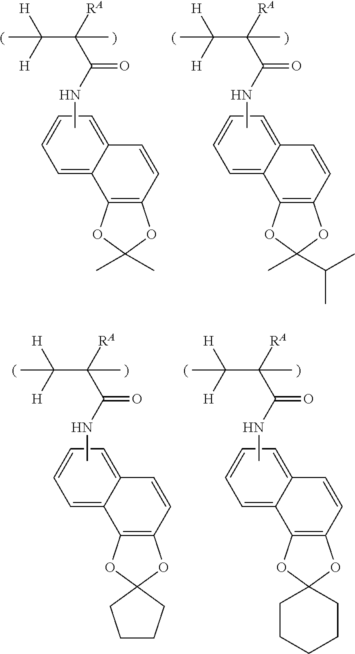

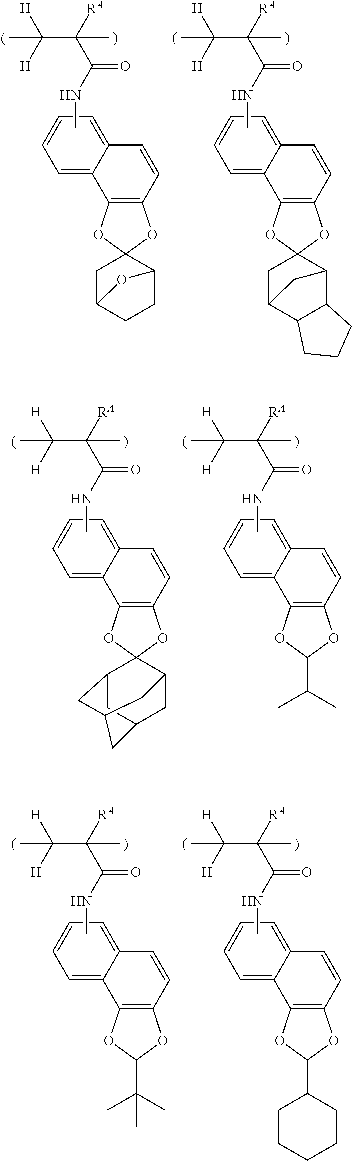

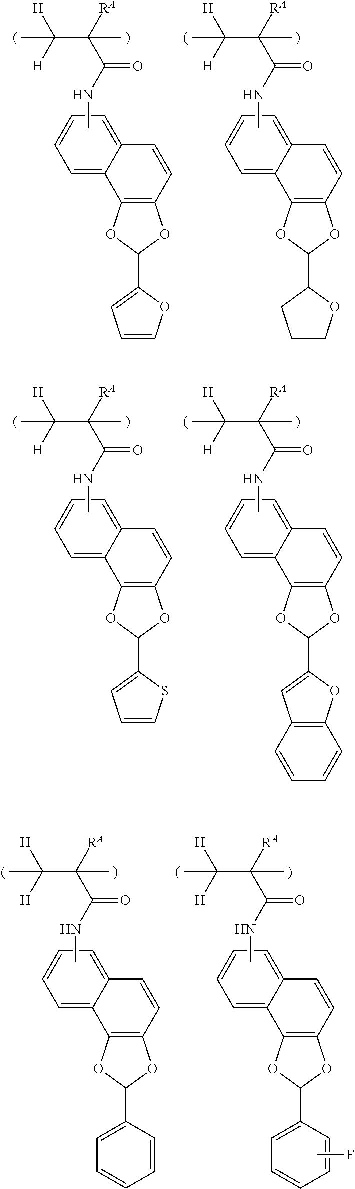

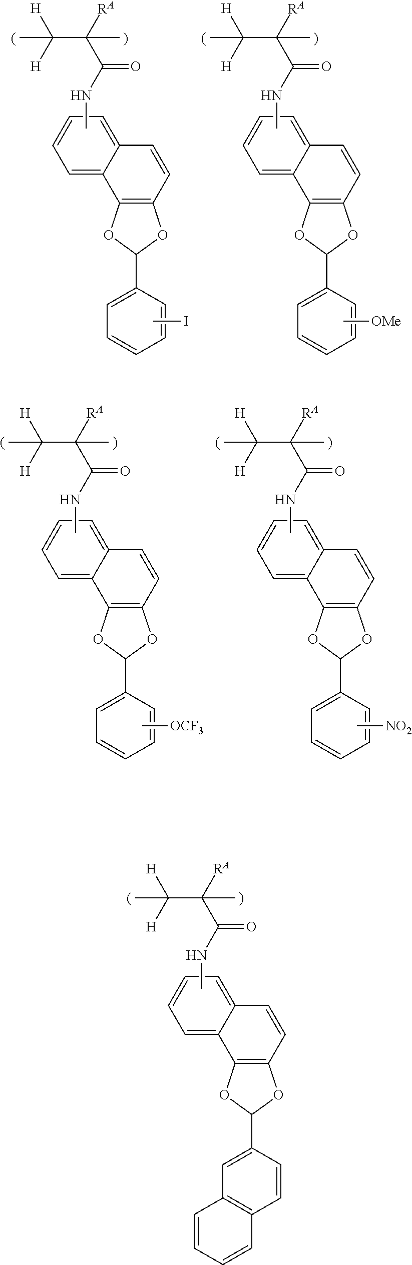

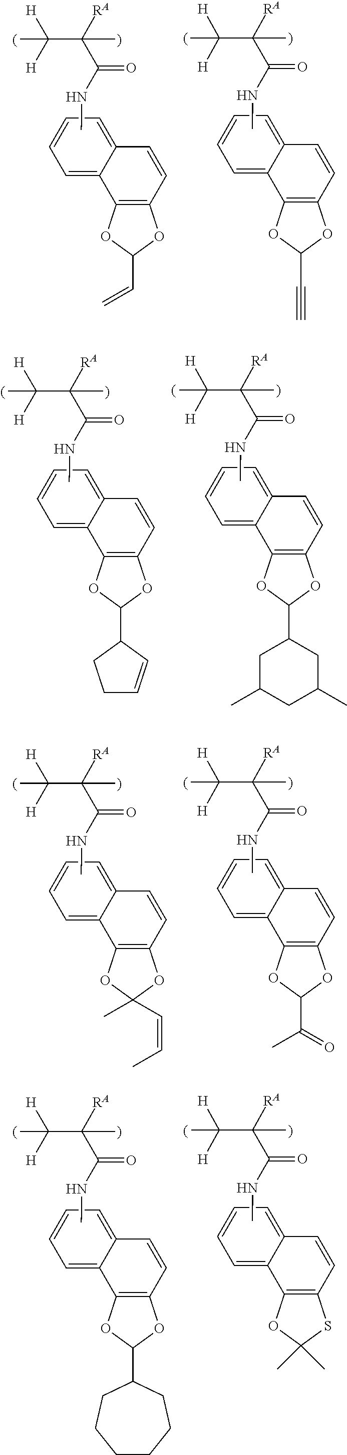

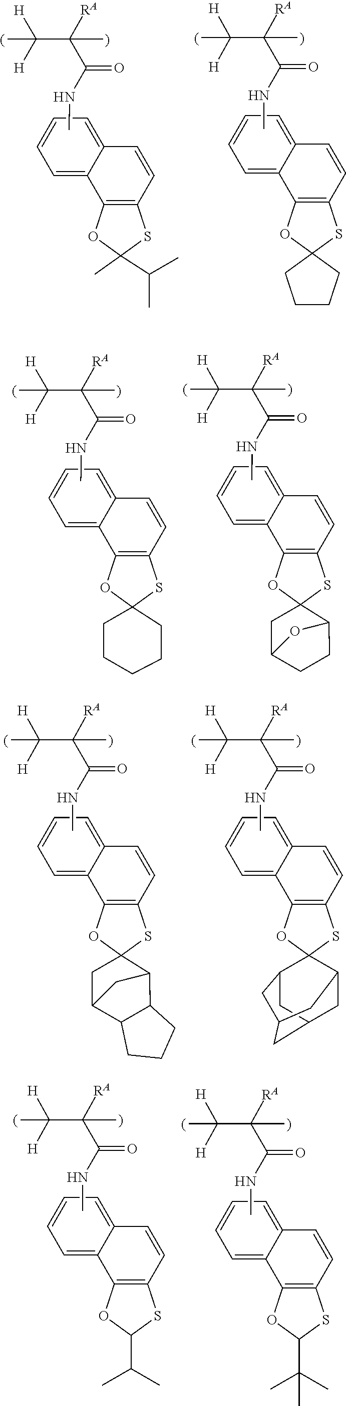

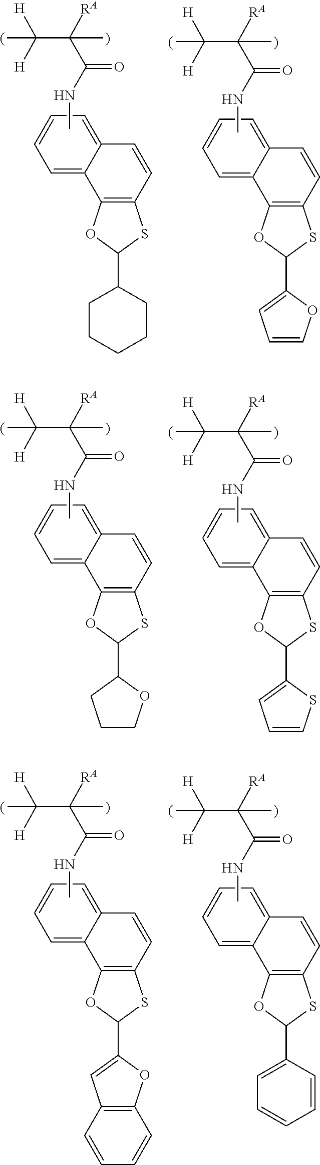

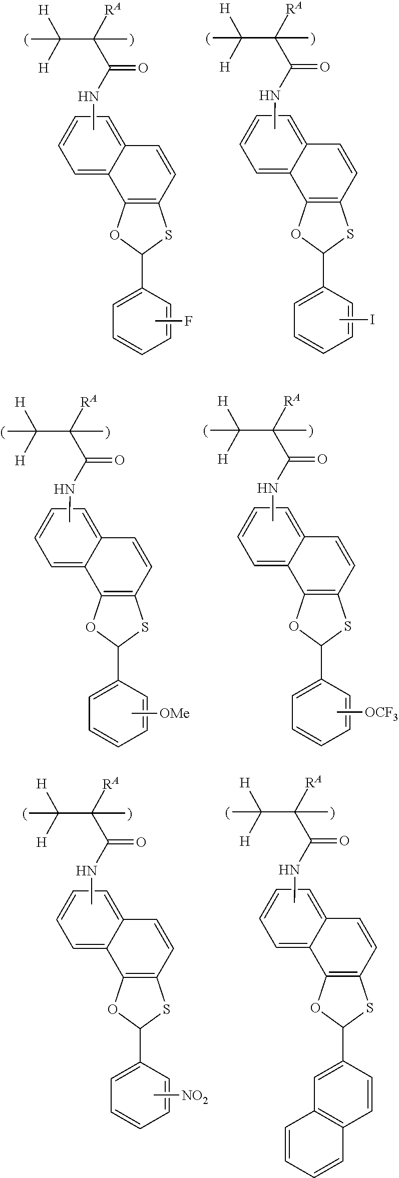

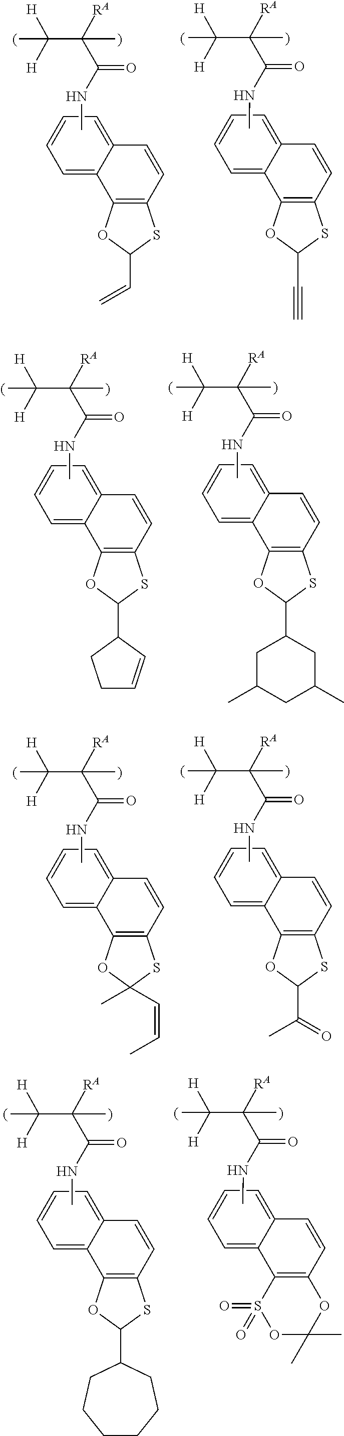

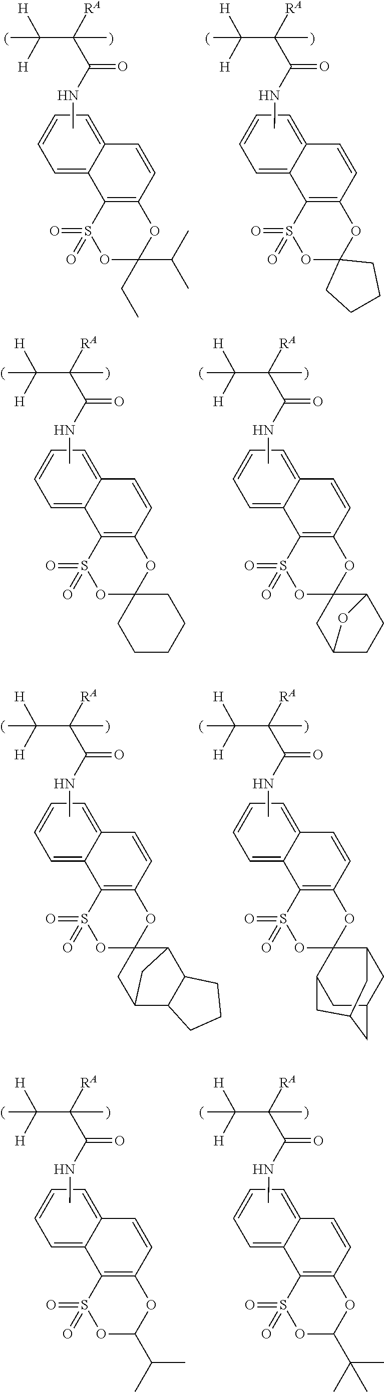

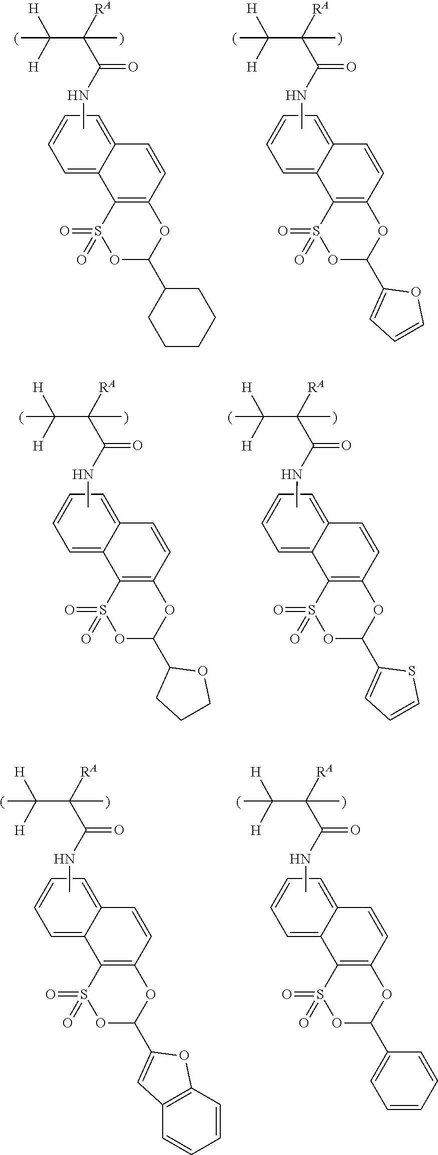

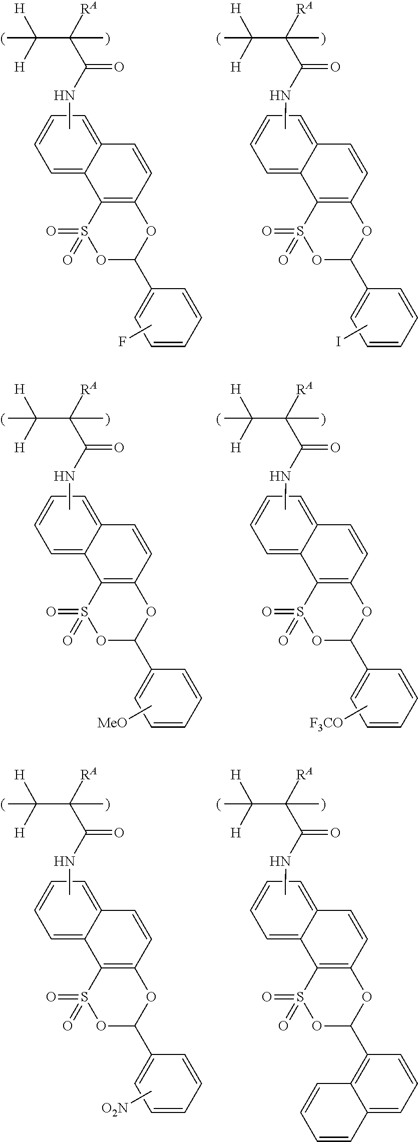

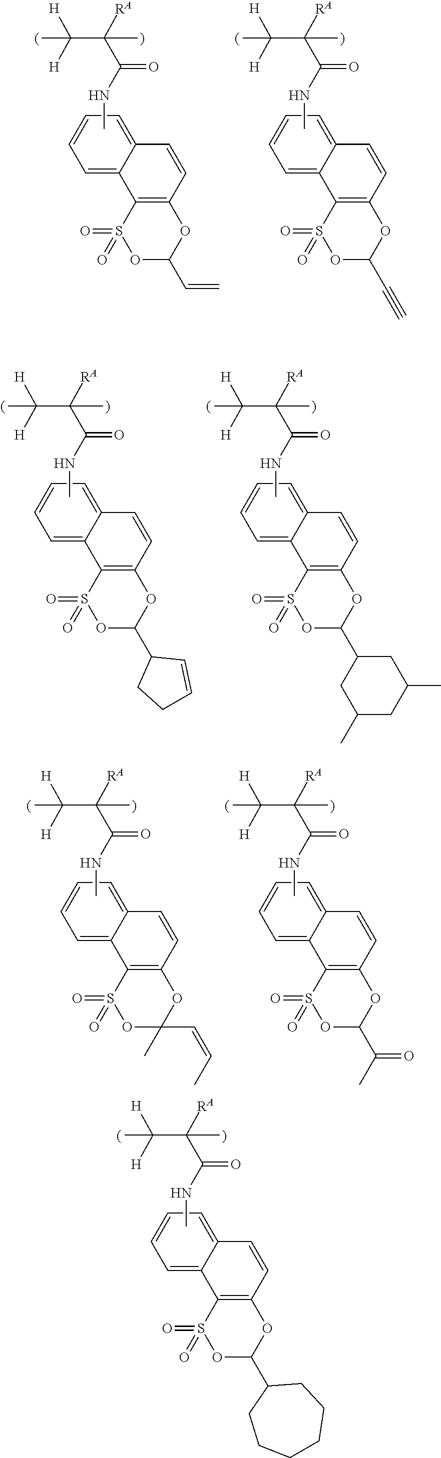

In a preferred embodiment, the base polymer further comprises repeat units having the formula (b1) or (b2), which are also referred to as repeat units (b1) or (b2).

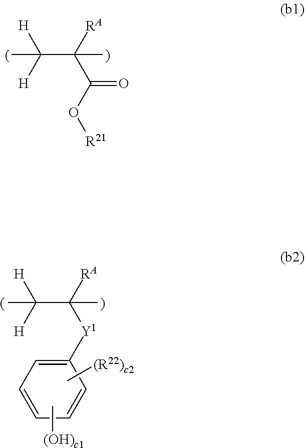

In formulae (b1) and (b2), R^{A} is each independently hydrogen, fluorine, methyl or trifluoromethyl. Y¹ is a single bond or *—C(═O)—O—, wherein * designates a point of attachment to the carbon atom in the backbone. R²¹ is hydrogen or a C₁-C₂₀ group containing at least one structure selected from hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—). R²² is halogen, carboxy, nitro, cyano, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom. When c2 is 2, 3 or 4, a plurality of R²² may be identical or different. The subscript c1 is 1, 2, 3 or 4, c2 is 0, 1, 2, 3 or 4, and 1≤c1+c2≤5.

Examples of repeat units (b1) are shown below, but not limited thereto. Herein R^{A} is as defined above.

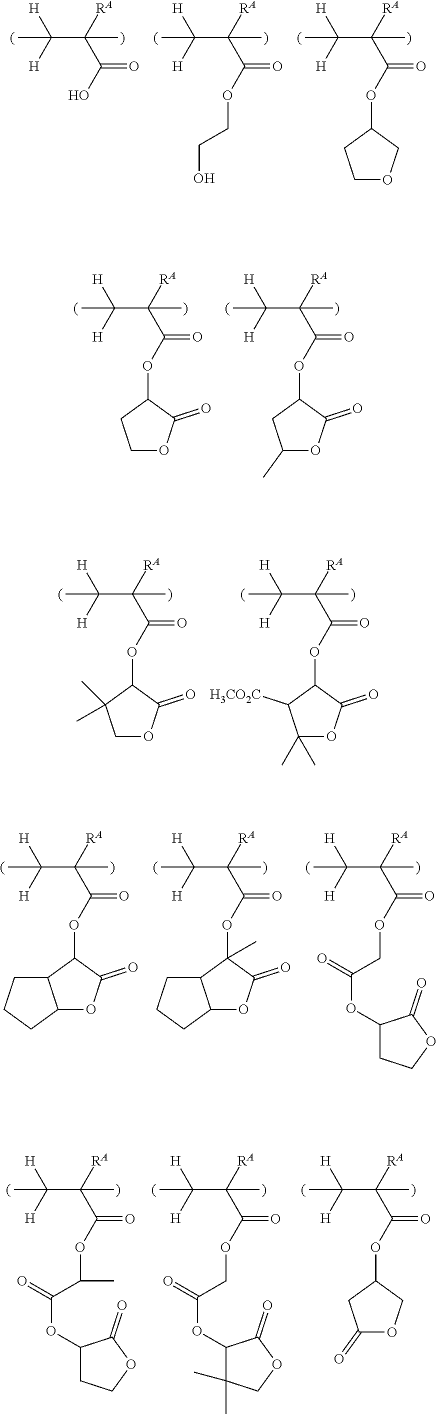

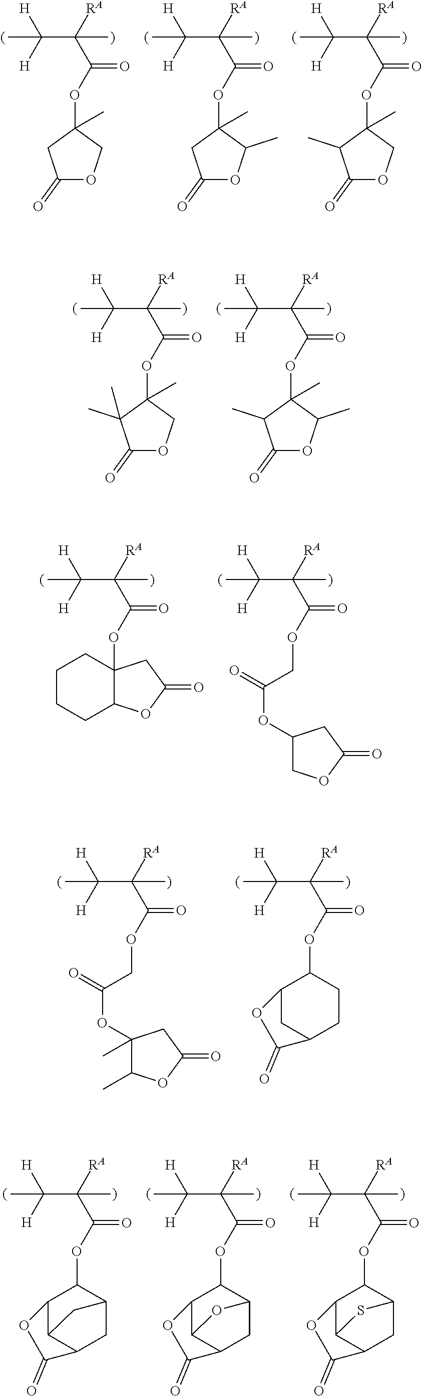

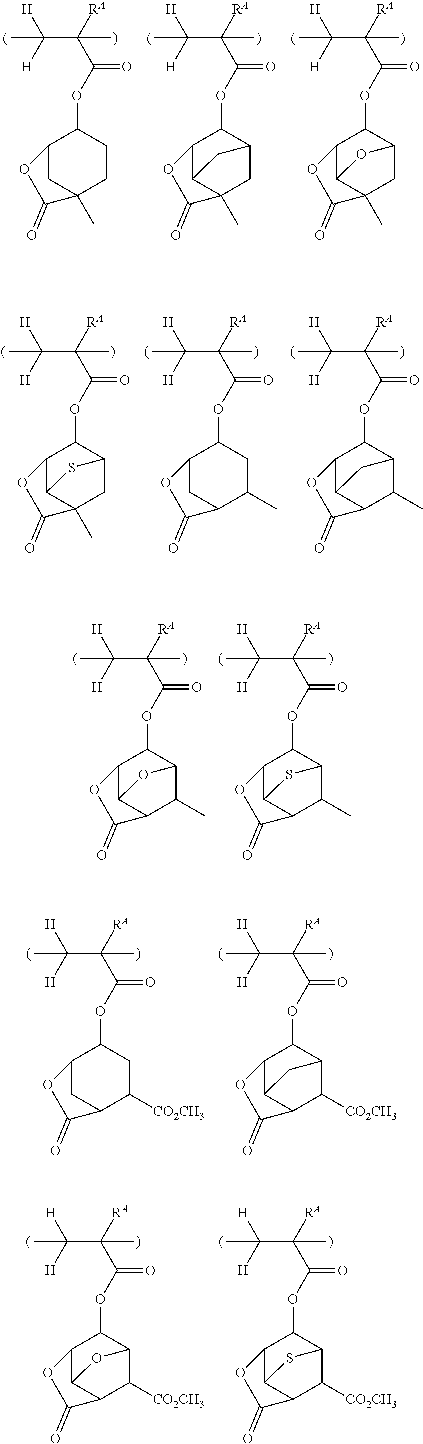

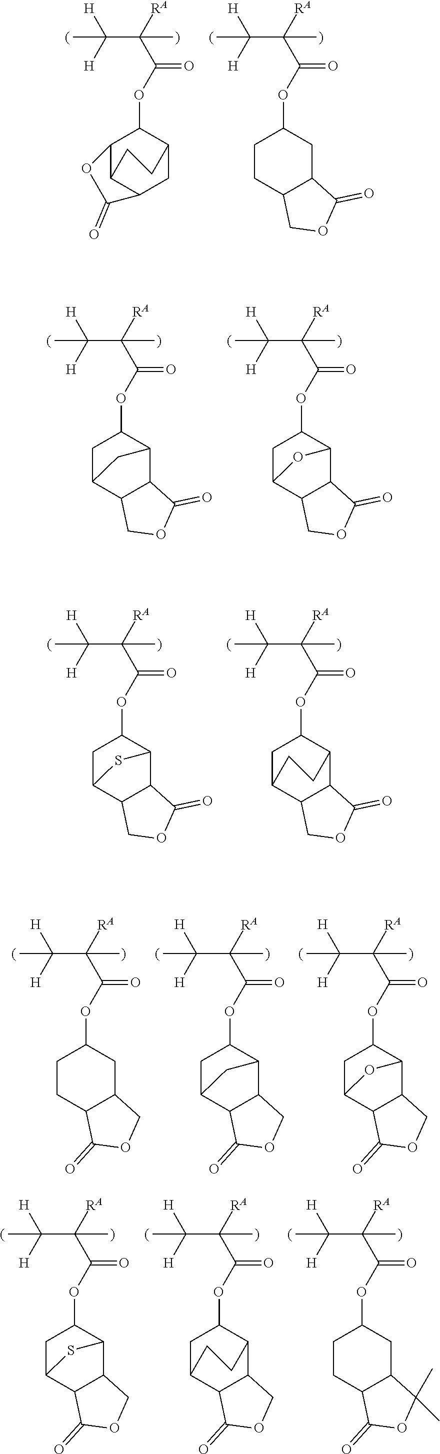

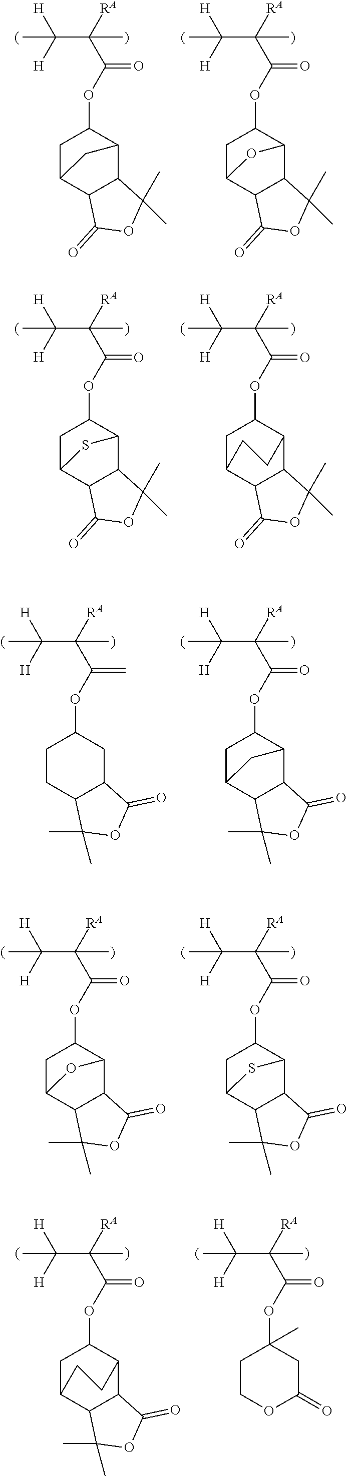

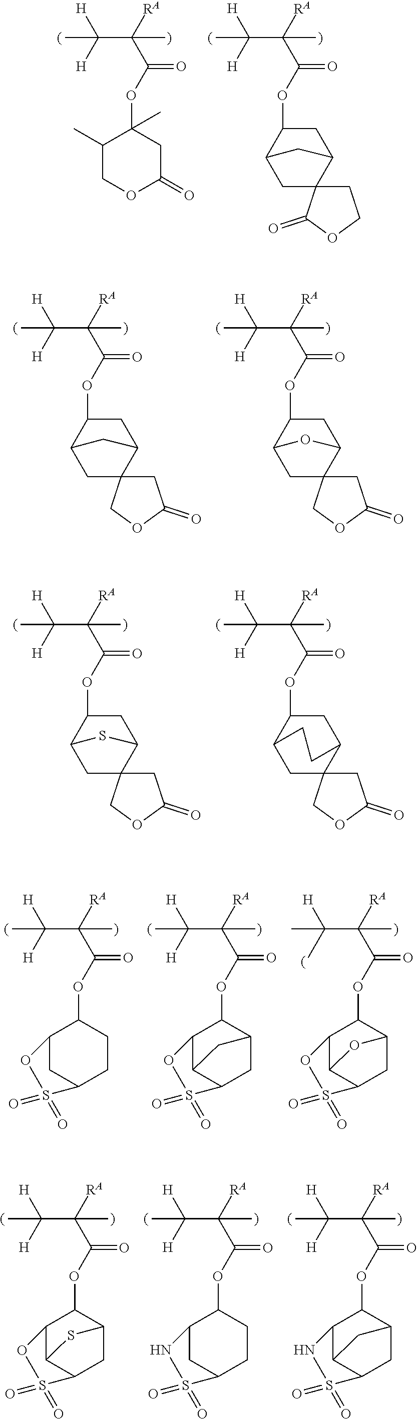

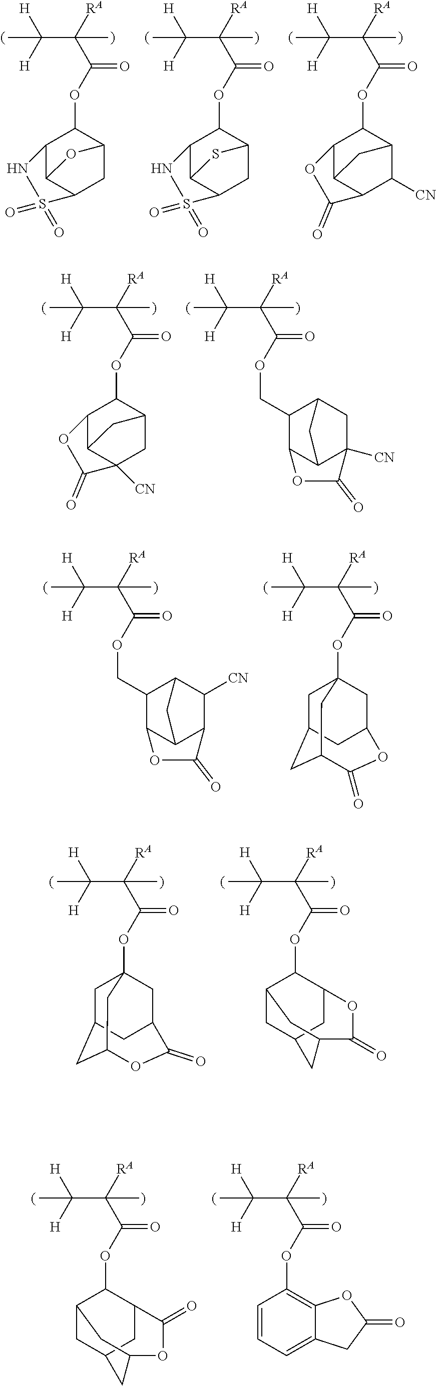

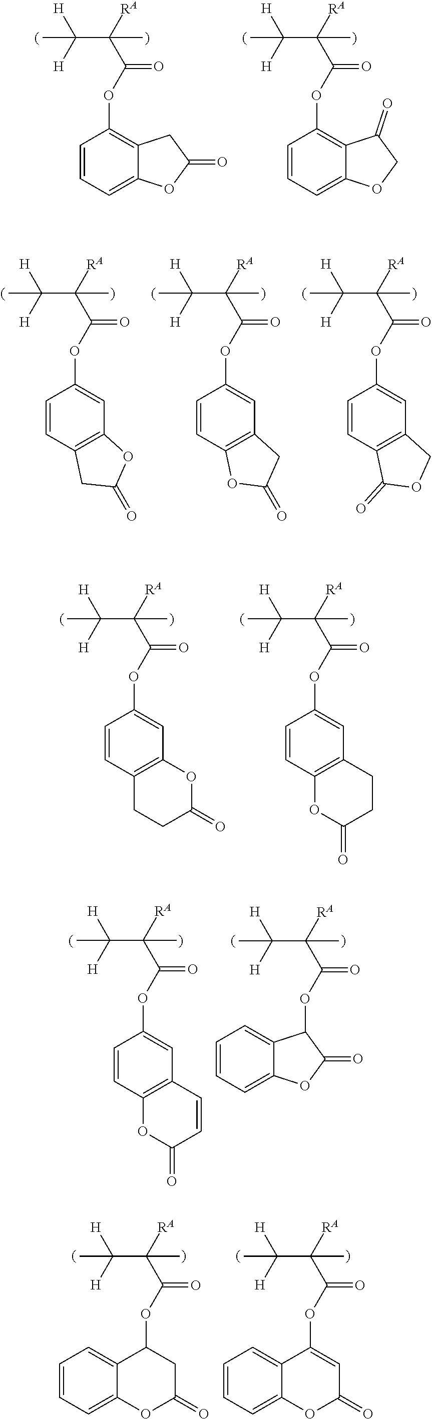

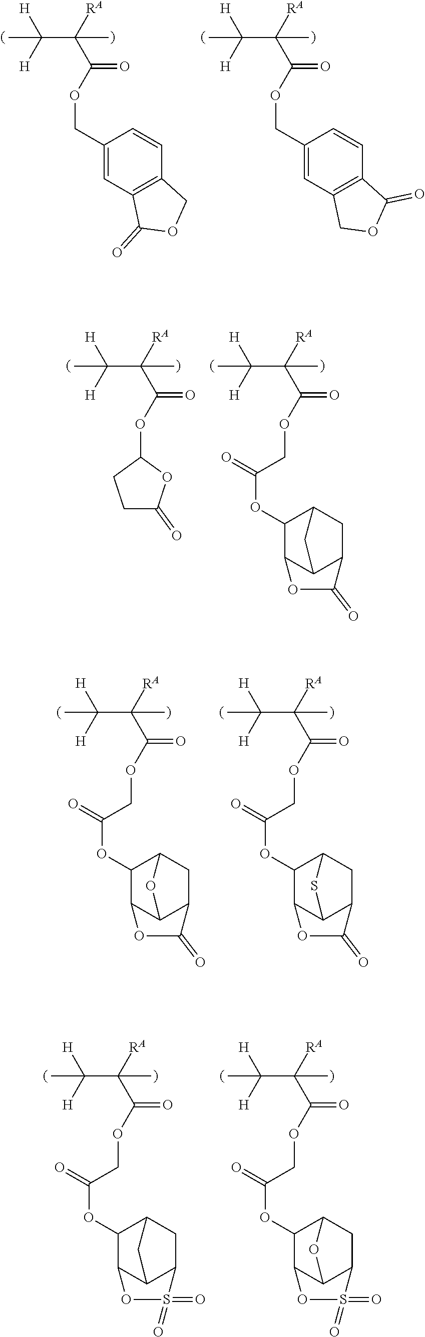

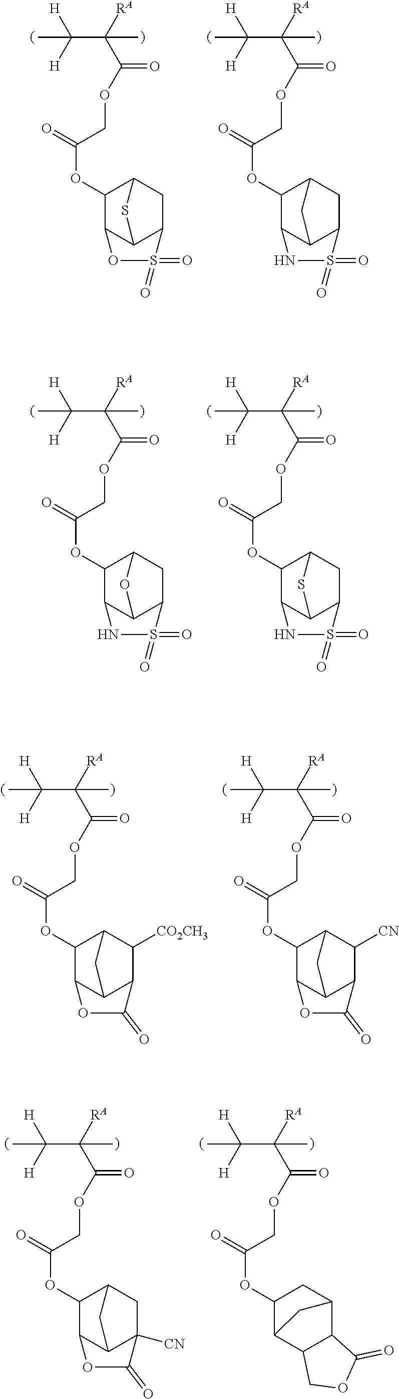

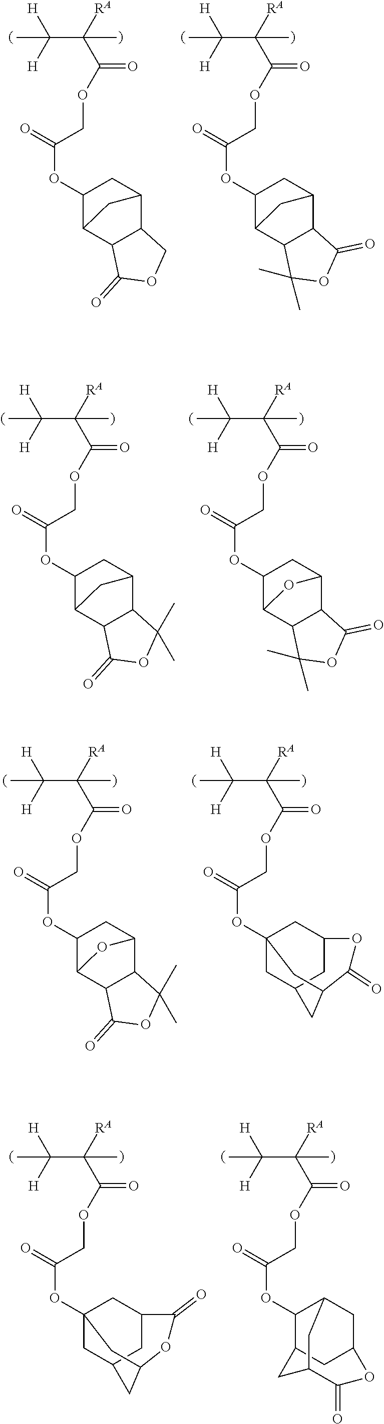

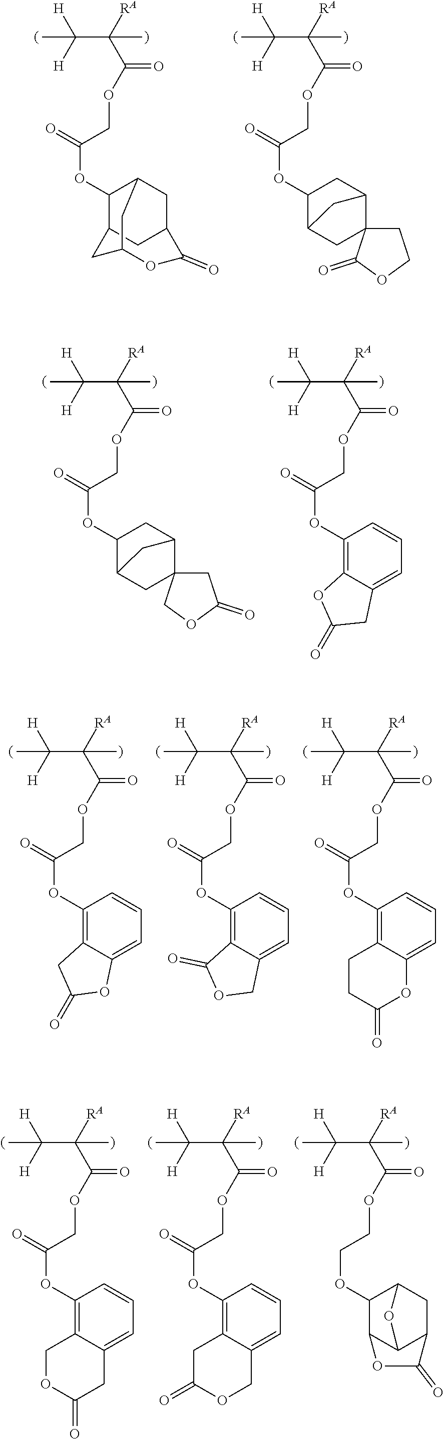

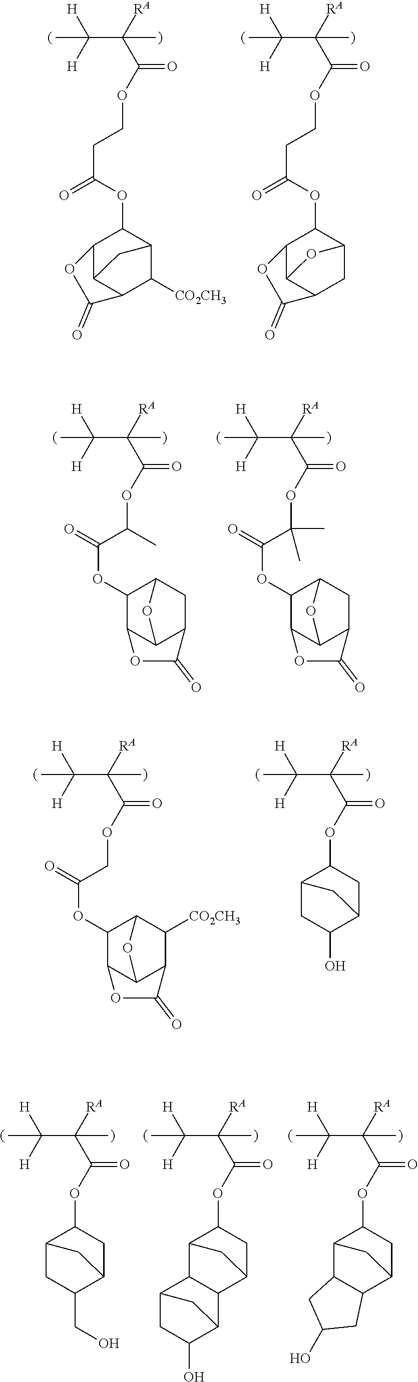

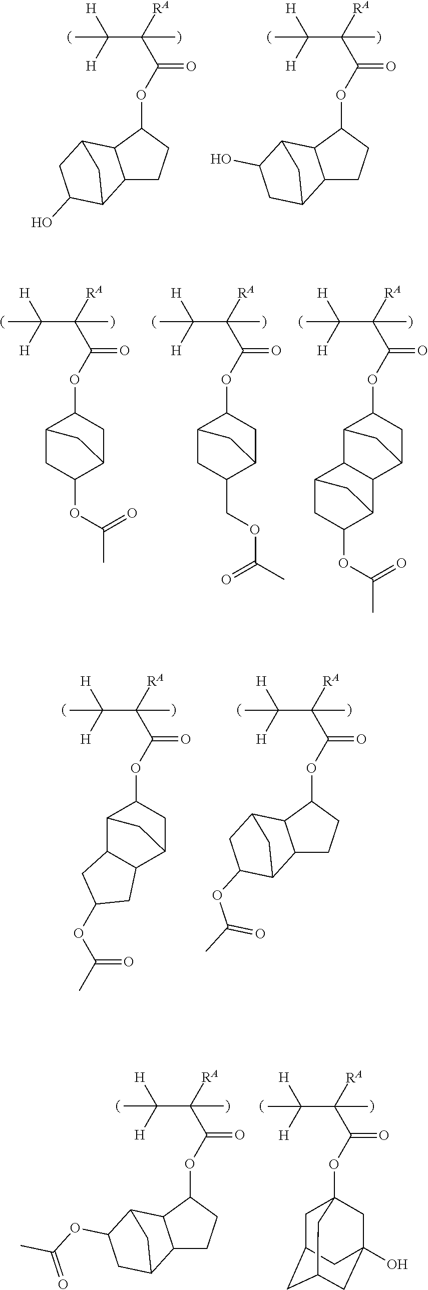

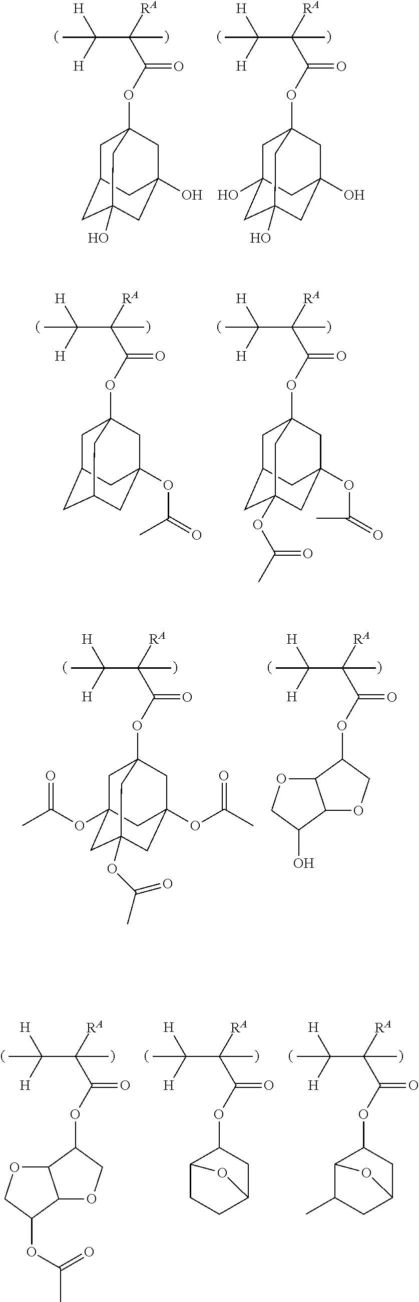

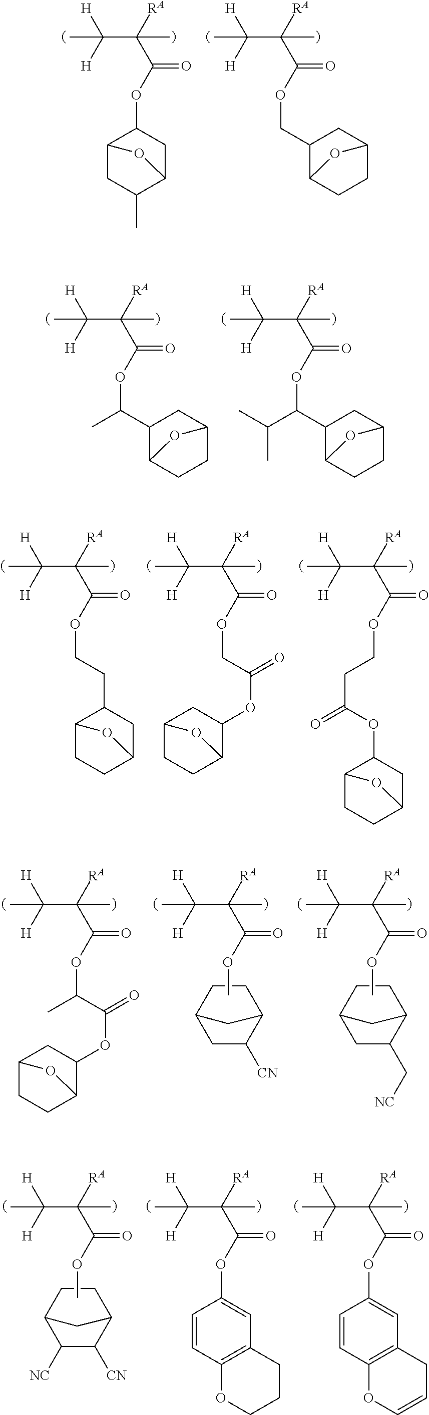

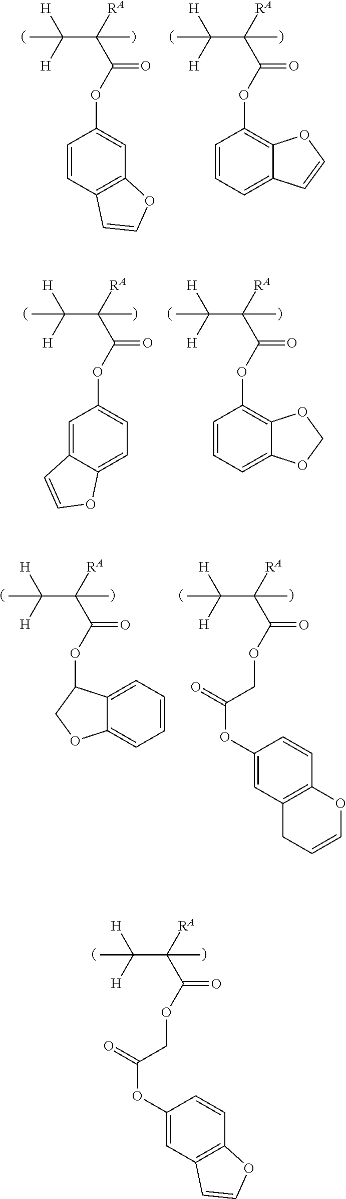

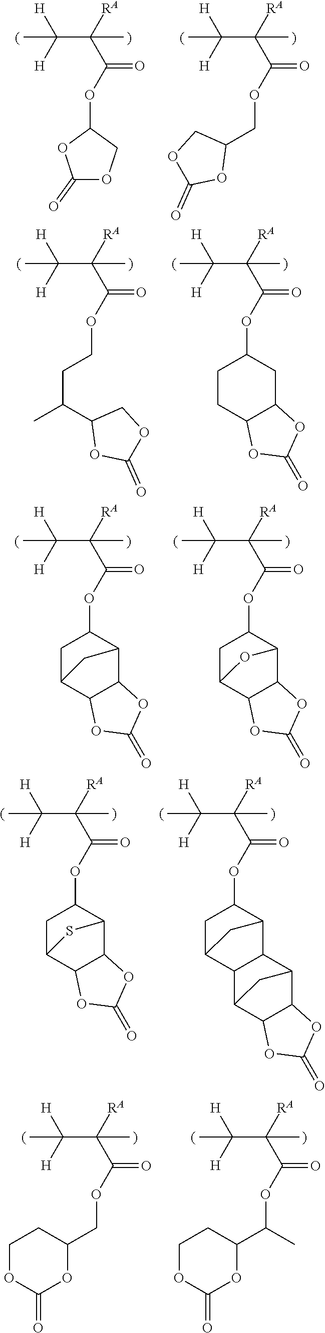

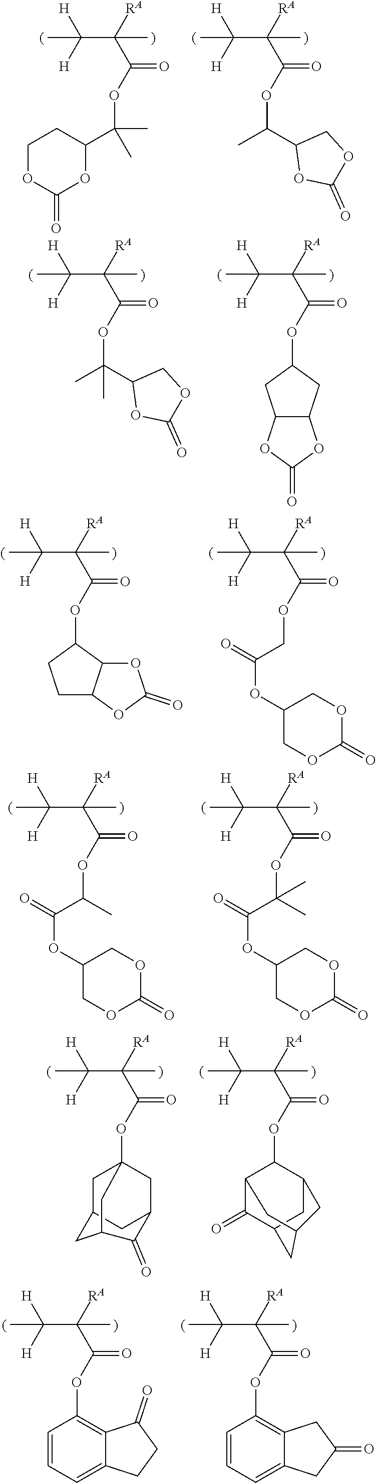

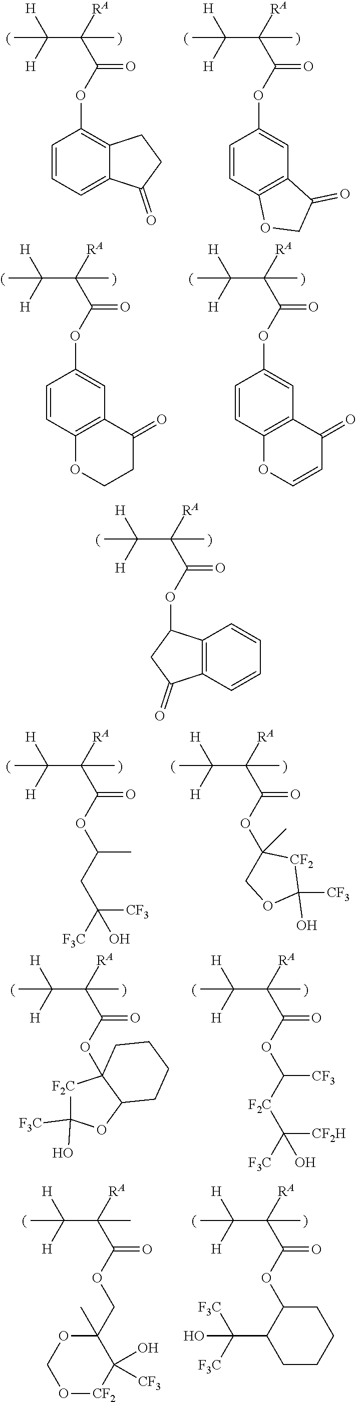

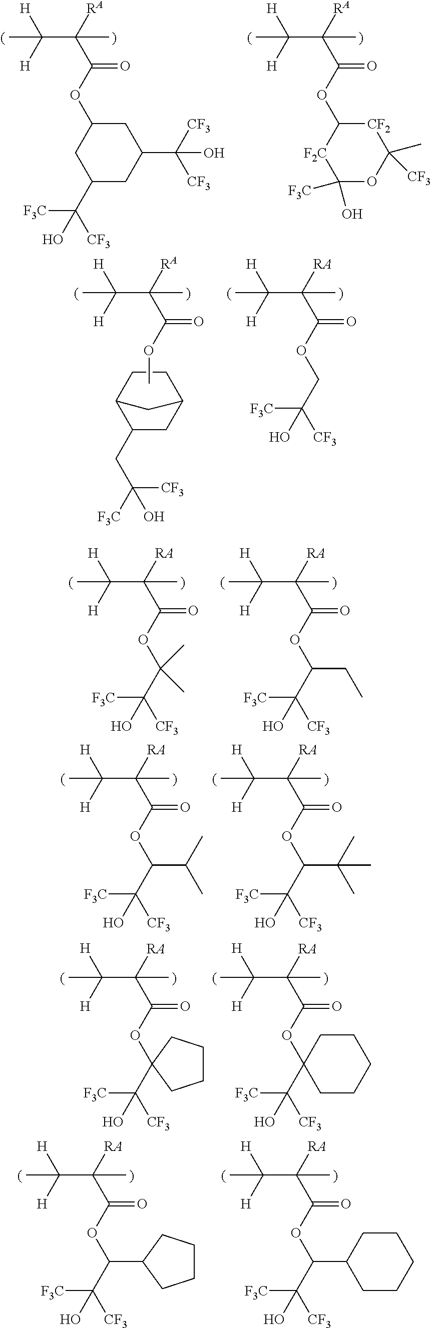

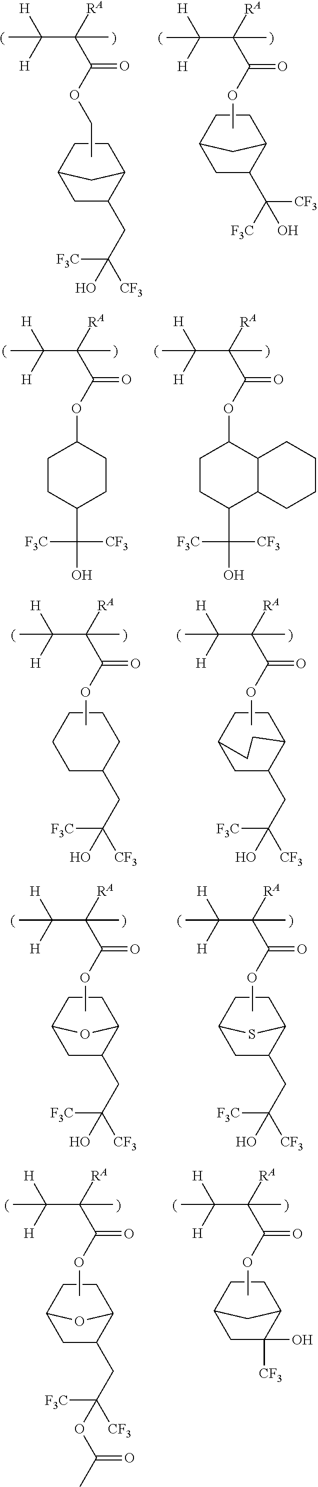

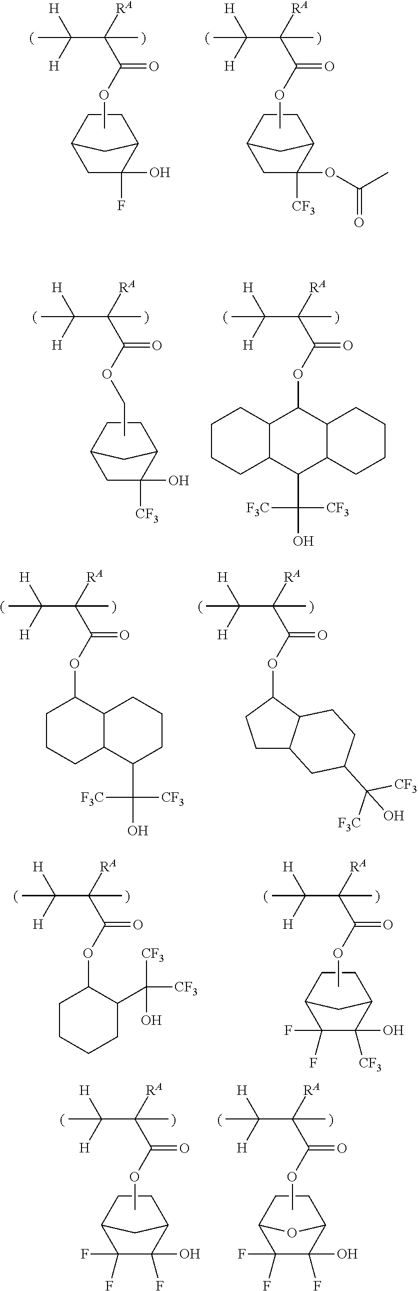

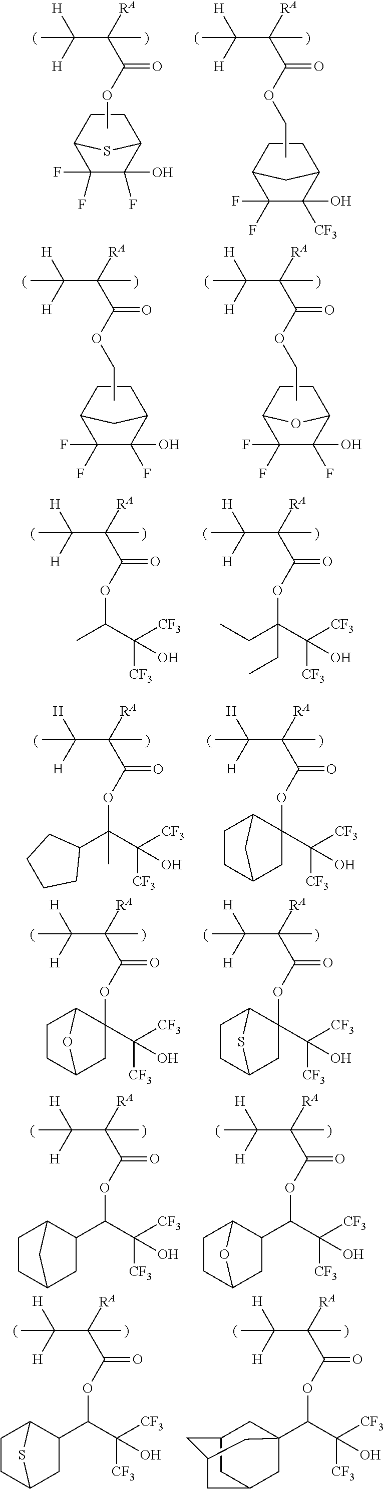

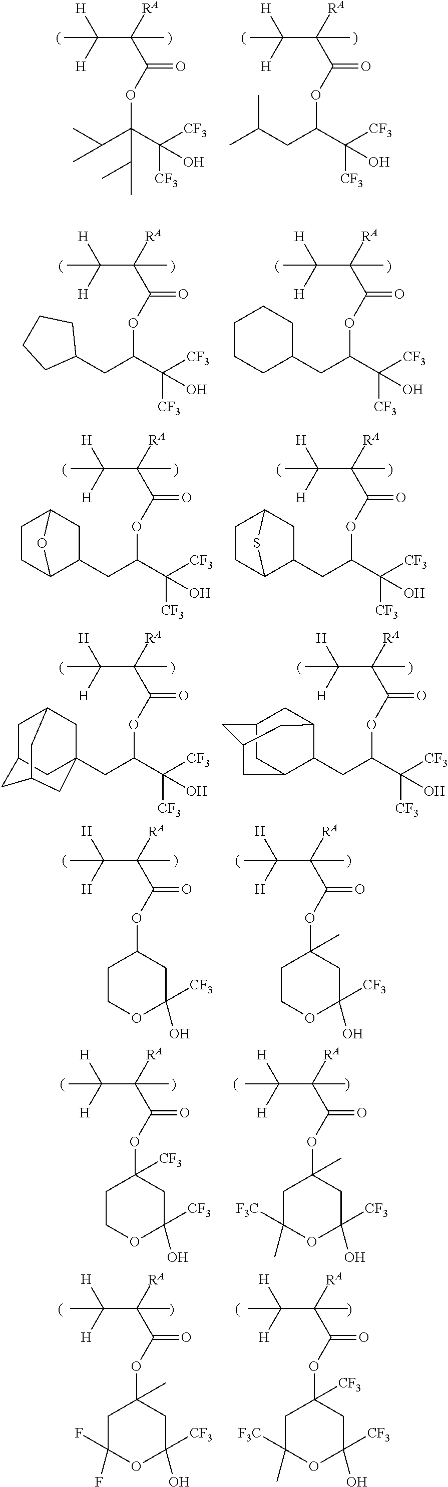

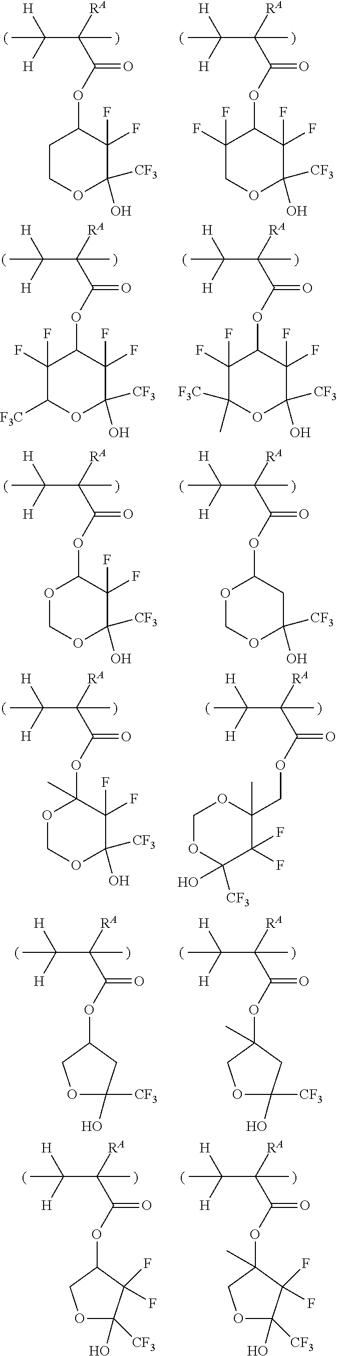

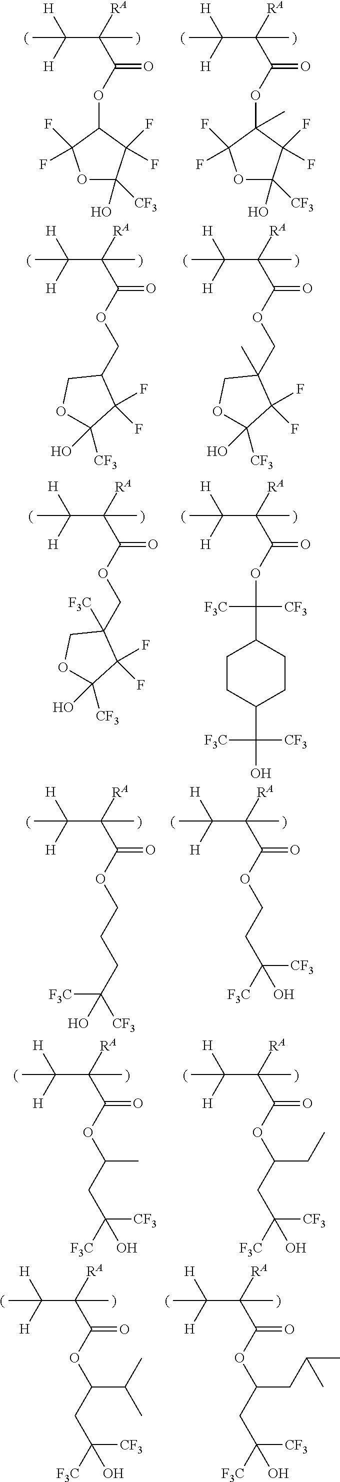

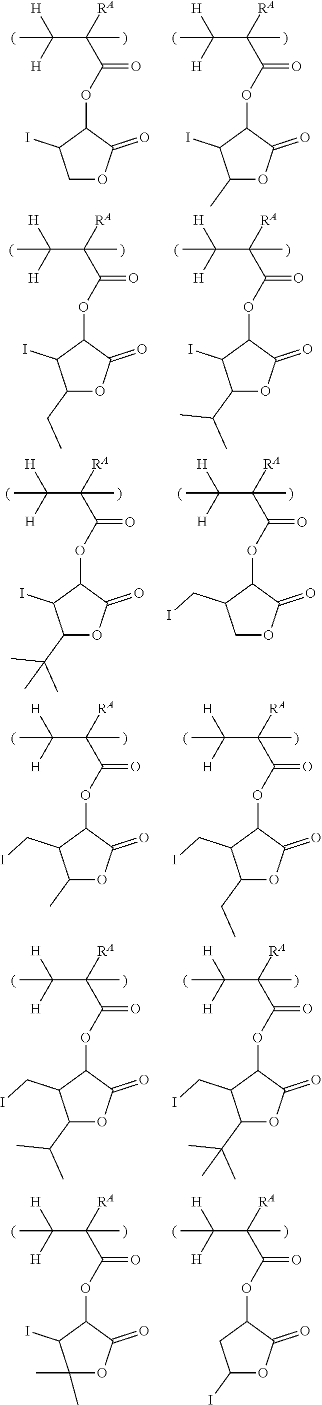

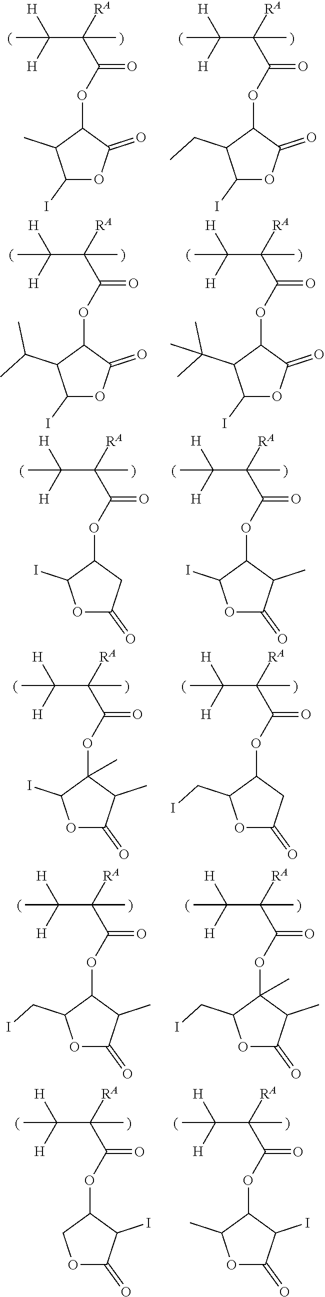

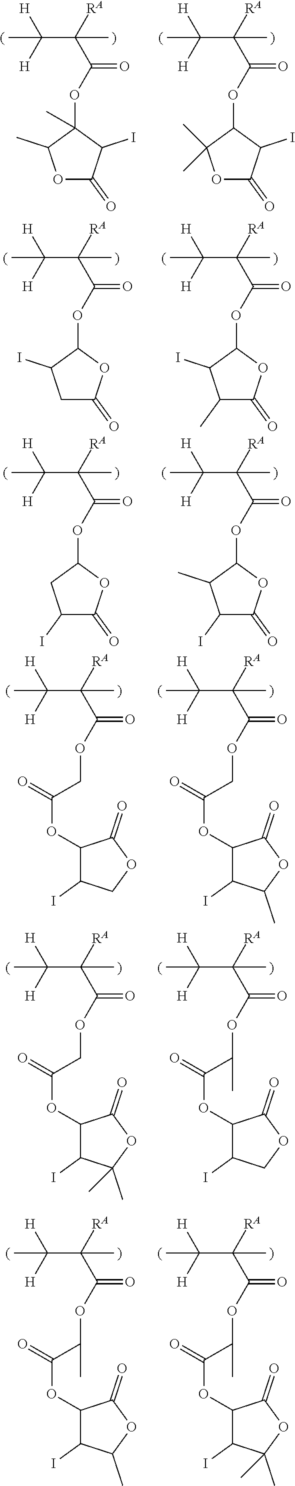

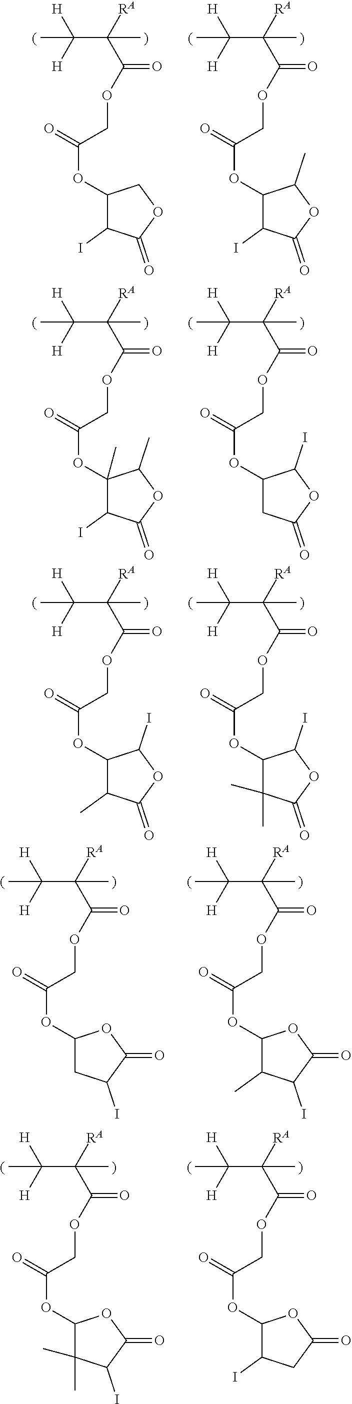

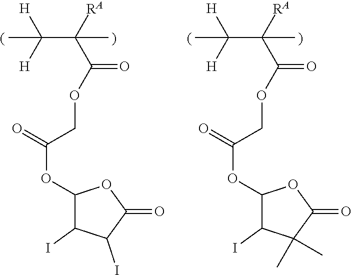

Examples of repeat units (b2) are shown below, but not limited thereto. Herein R^{A} is as defined above.

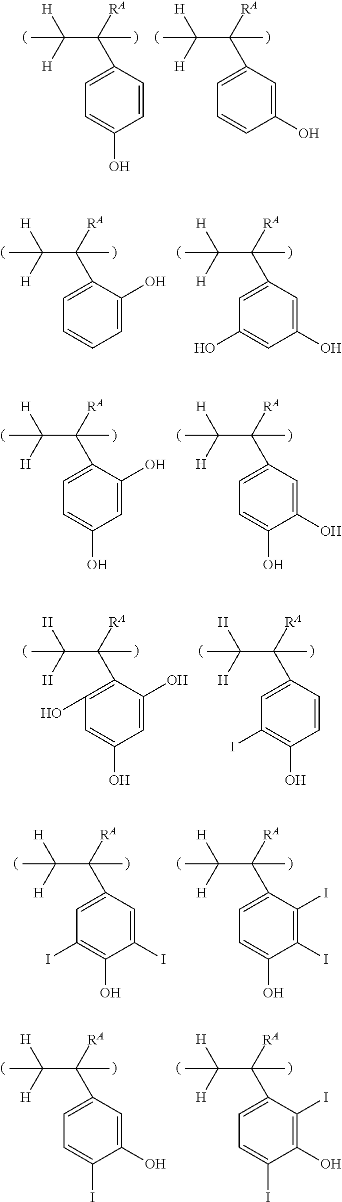

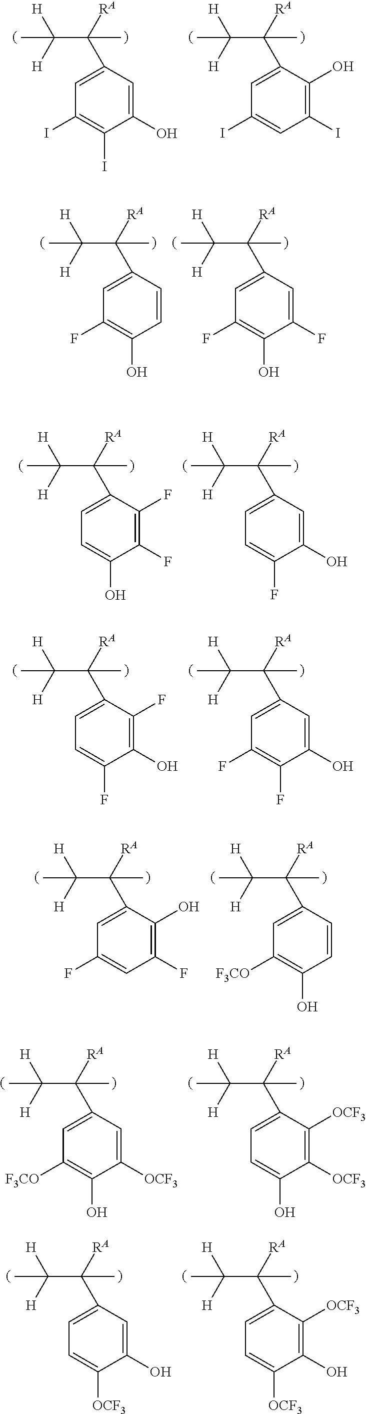

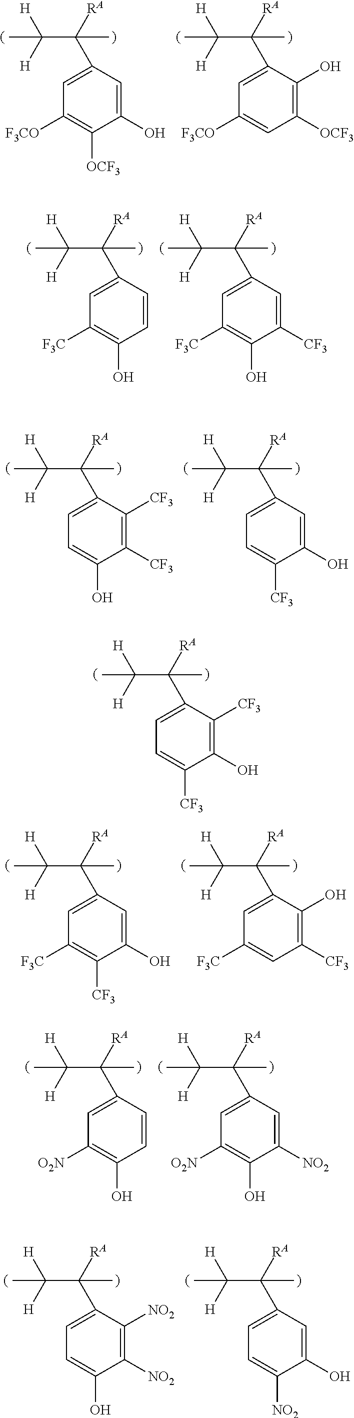

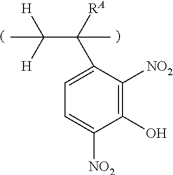

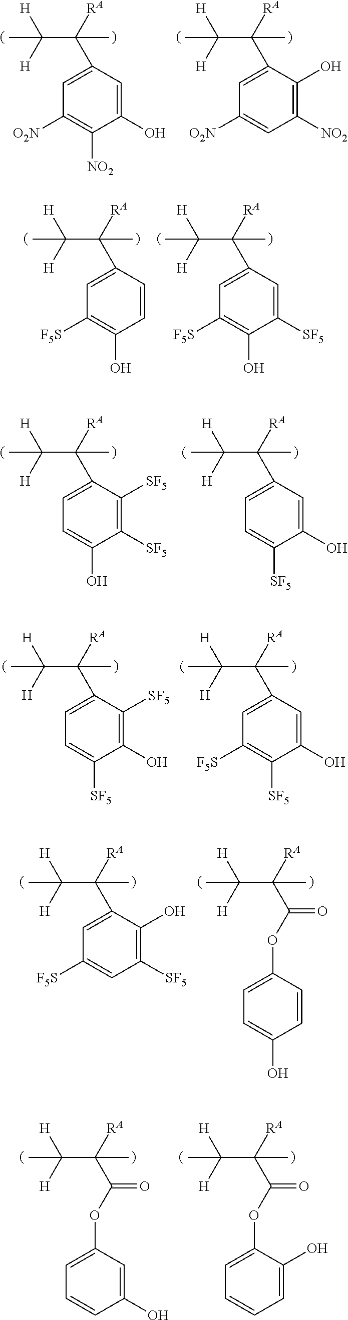

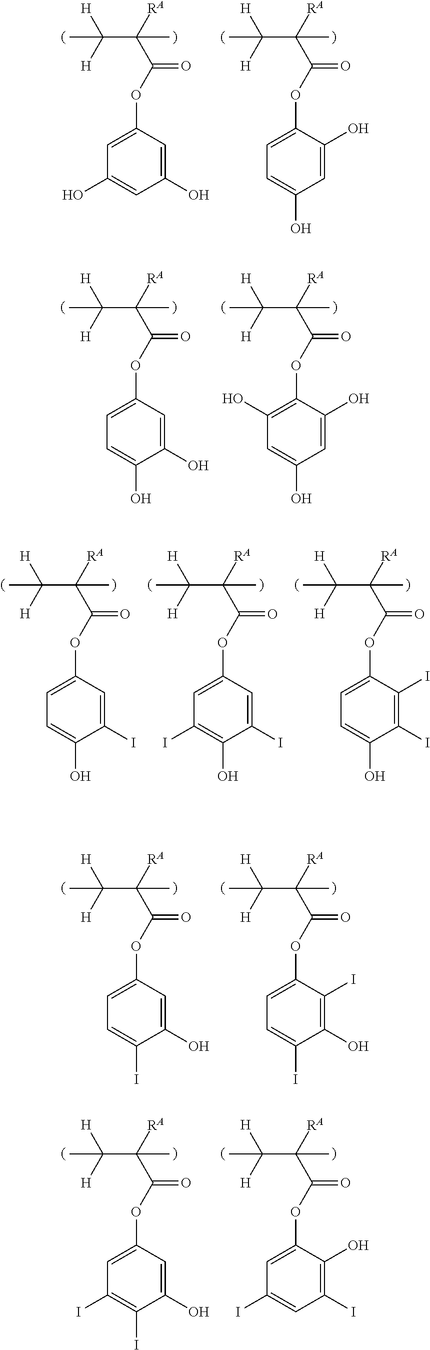

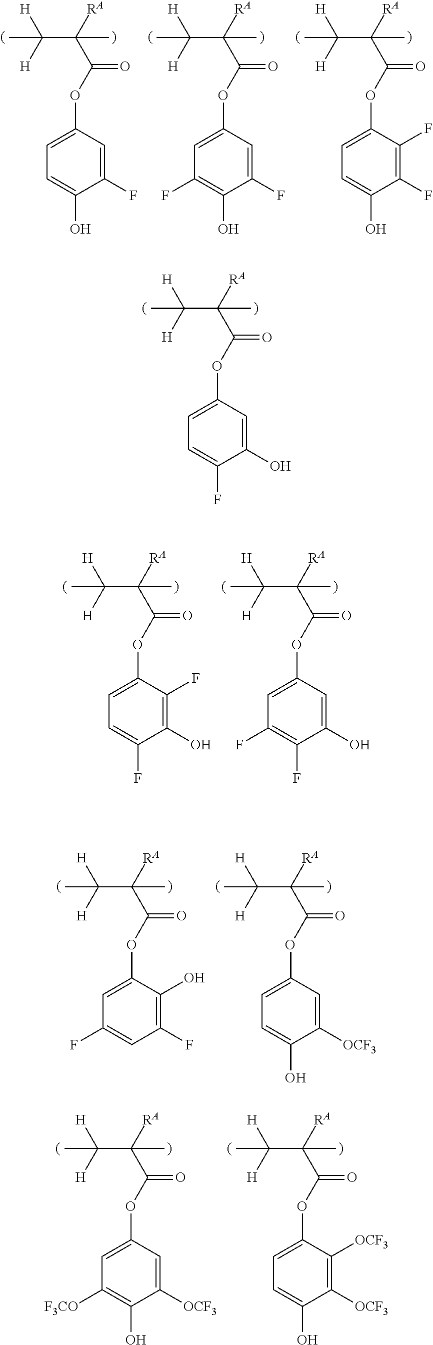

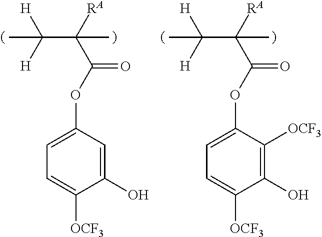

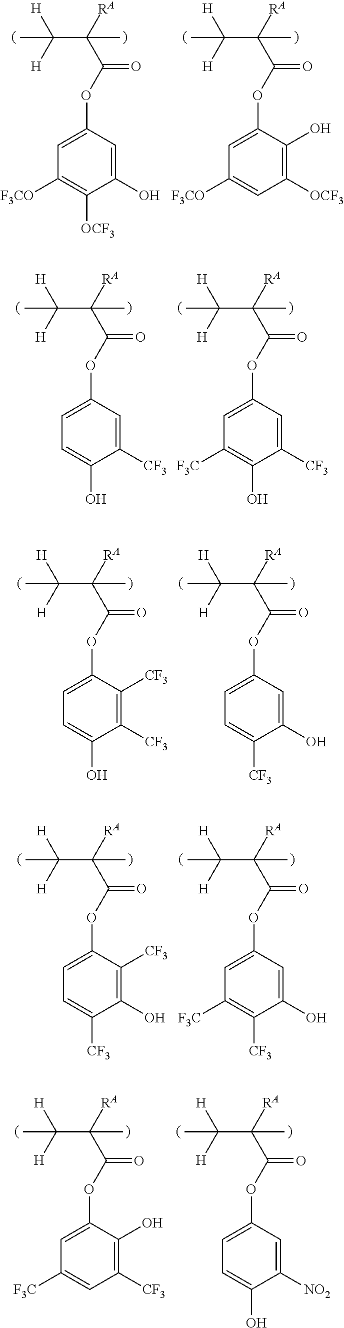

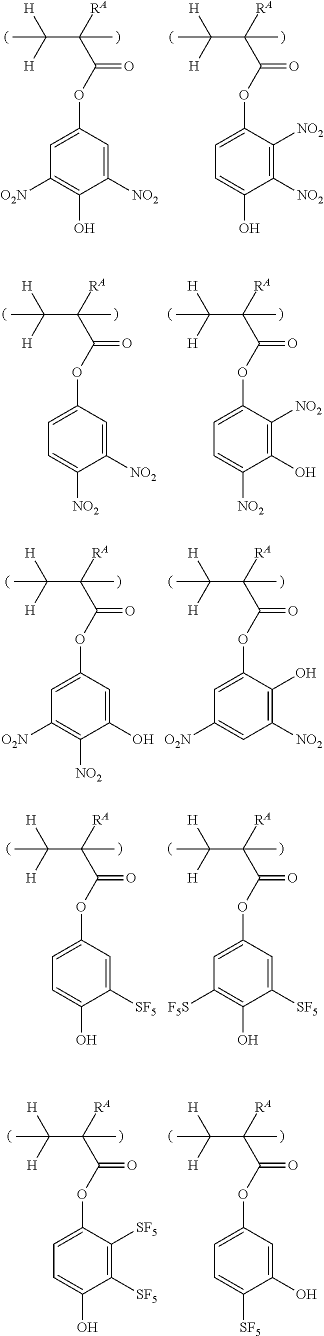

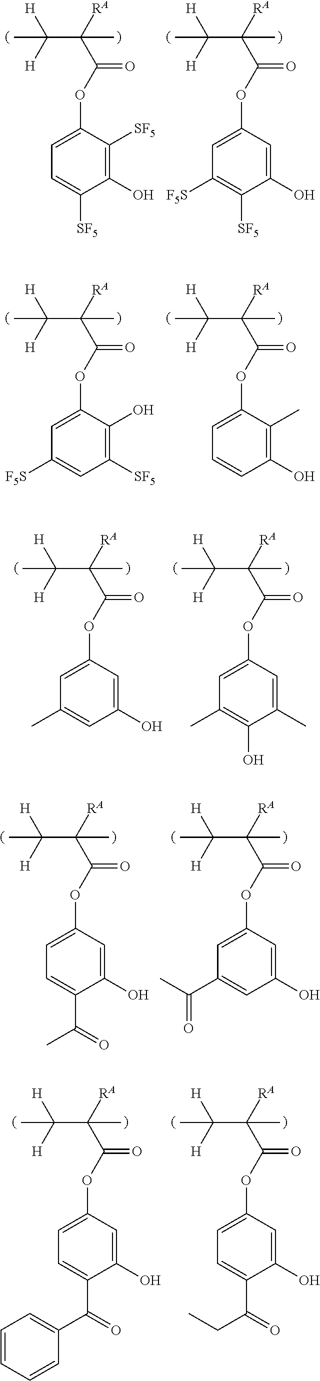

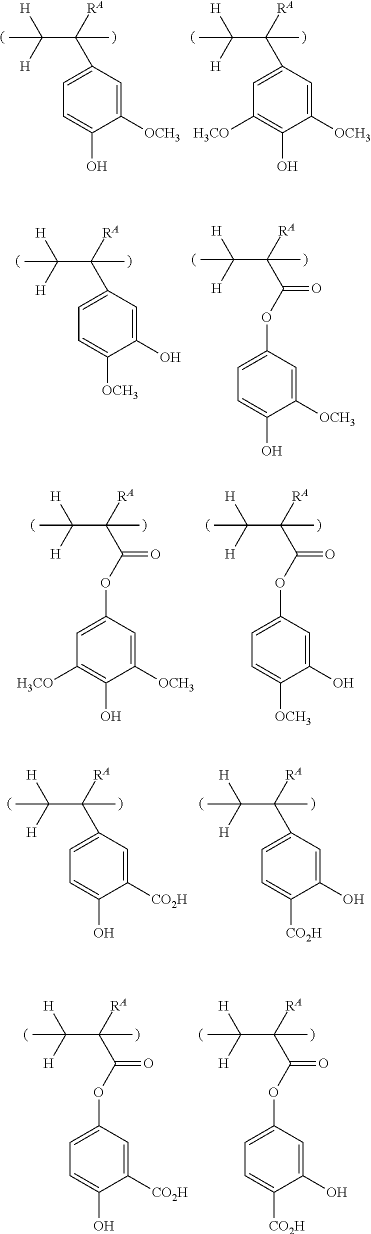

Of the repeat units (b1) and (b2), those units having a lactone ring as the polar group are preferred in the case of ArF lithography, and those units having a phenol site are preferred in the case of KrF, EB or EUV lithography.

The base polymer may further comprise repeat units of at least one type selected from repeat units having the formula (c1), repeat units having the formula (c2), repeat units having the formula (c3), repeat units having the formula (c4), and repeat units having the formula (c5), which are simply referred to as repeat units (c1) to (c5).

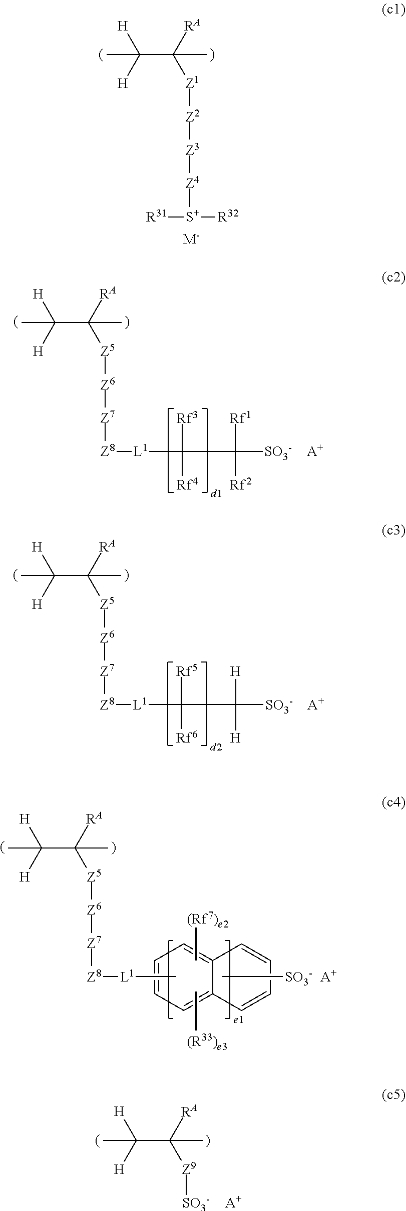

In formulae (c1) to (c5), R^{A} is each independently hydrogen, fluorine, methyl or trifluoromethyl.

Z¹ is a single bond or optionally substituted phenylene group. Z² is a single bond, **—C(═O)—O—Z²¹—, **—C(═O)—NH—Z²¹—, or **—O—Z²¹—. Z²¹ is a C₁-C₆ aliphatic hydrocarbylene group, phenylene group or a divalent group obtained by combining the foregoing, which may contain halogen, carbonyl moiety, ester bond, ether bond or hydroxy moiety. Z³ is a single bond, ether bond, ester bond, sulfonate ester bond, amide bond, sulfonamide bond, carbonate bond or carbamate bond. Z⁴ is a single bond or a C₁-C₆ aliphatic hydrocarbylene group, phenylene group or a divalent group obtained by combining the foregoing, which may contain halogen, carbonyl moiety, ester bond, ether bond or hydroxy moiety.

Z⁵ is each independently a single bond, optionally substituted phenylene group, naphthylene group or *—C(═O)—O—Z⁵¹—. Z⁵¹ is a C₁-C₁₀ aliphatic hydrocarbylene group, phenylene group or naphthylene group, the aliphatic hydrocarbylene group may contain halogen, hydroxy, ether bond, ester bond or lactone ring. Z⁶ is a single bond, ether bond, ester bond, sulfonate ester bond, amide bond, sulfonamide bond, carbonate bond or carbamate bond. Z⁷ is each independently a single bond, ***—Z⁷¹—C(═O)—O—, ***—C(═O)—NH—Z⁷¹— or ***—O—Z⁷¹—. Z⁷¹ is a C₁-C₂₀ hydrocarbylene group which may contain a heteroatom. Z⁸ is each independently a single bond, ****—Z⁸—C(═O)—O—, ****—C(═O)—NH—Z⁸¹— or ****—O—Z^{8l}—. Z⁸¹ is a C₁-C₂₀ hydrocarbylene group which may contain a heteroatom.

Z⁹ is a single bond, methylene, ethylene, phenylene, fluorinated phenylene, trifluoromethyl-substituted phenylene, *—C(═O)—O—Z⁹¹—, *—C(═O)—N(H)—Z⁹¹— or *—O—Z⁹¹—. Z⁹¹ is a C₁-C₆ aliphatic hydrocarbylene group, phenylene group, fluorinated phenylene group, or trifluoromethyl-substituted phenylene group, which may contain carbonyl, ester bond, ether bond or hydroxy.

Herein, * designates a point of attachment to the carbon atom in the backbone, ** designates a point of attachment to Z¹, *** designates a point of attachment to Z⁶, **** designates a point of attachment to Z⁷.

The aliphatic hydrocarbylene group represented by Z²¹, Z⁵¹ and Z⁹¹ may be straight, branched or cyclic. Examples thereof include alkanediyl groups such as methanediyl, ethane-1,1-diyl, ethane-1,2-diyl, propane-1,1-diyl, propane-1,2-diyl, propane-1,3-diyl, propane-2,2-diyl, butane-1,1-diyl, butane-1,2-diyl, butane-1,3-diyl, butane-2,3-diyl, butane-1,4-diyl, 1,1-dimethylethane-1,2-diyl, pentane-1,5-diyl, 2-methylbutane-1,2-diyl, and hexane-1,6-diyl; cycloalkanediyl groups such as cyclopropanediyl, cyclobutanediyl, cyclopentanediyl and cyclohexanediyl, and combinations thereof.

The hydrocarbylene group which may contain a heteroatom, represented by Z⁷¹ and Z⁸¹, may be saturated or unsaturated and straight, branched or cyclic. Examples thereof are shown below, but not limited thereto.

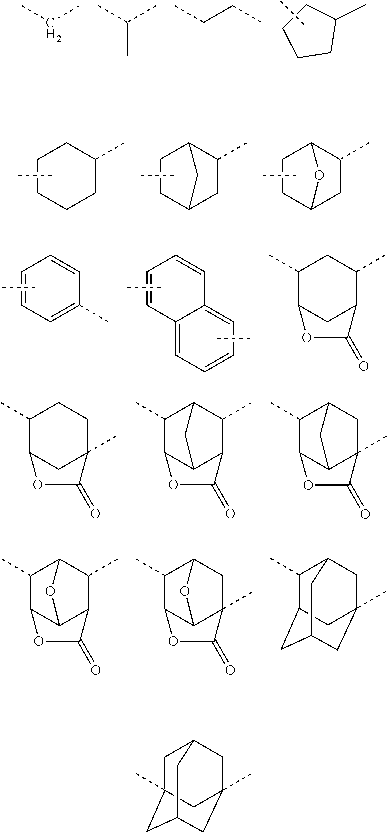

In formula (c1), R³¹ and R³² are each independently a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, and tert-butyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl, naphthyl and thienyl; C₇-C₂₀ aralkyl groups such as benzyl, 1-phenylethyl, and 2-phenylethyl, and combinations thereof. Of these, aryl groups are preferred. In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonate ester bond, carbonate bond, lactone ring, sultone ring, carboxylic anhydride (—C(═O)—O—C(═O)—) or haloalkyl moiety.

R³¹ and R³² may bond together to form a ring with the sulfur atom to which they are attached. Examples of the ring are shown below.

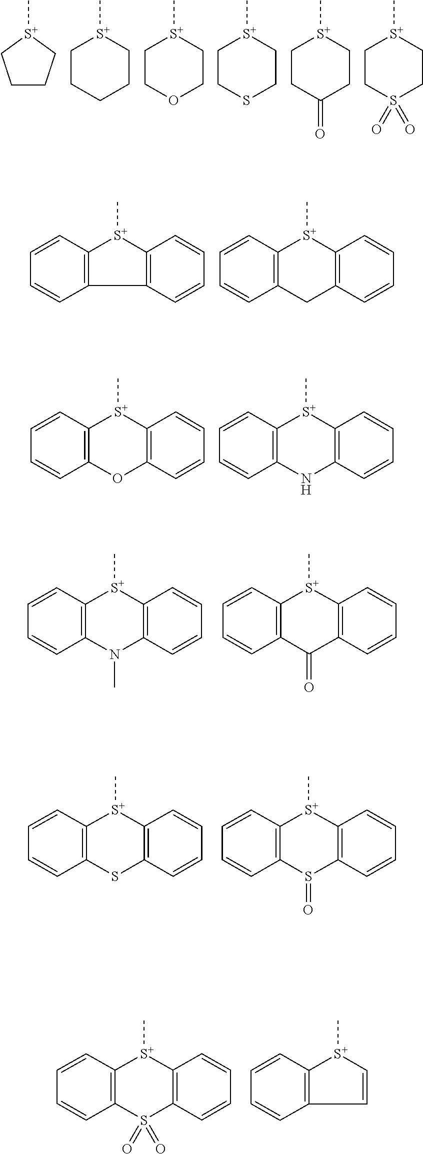

The broken line designates a point of attachment to Z⁴.

Examples of the cation in repeat unit (c1) are given below, but not limited thereto. Herein R^{A} is as defined above.

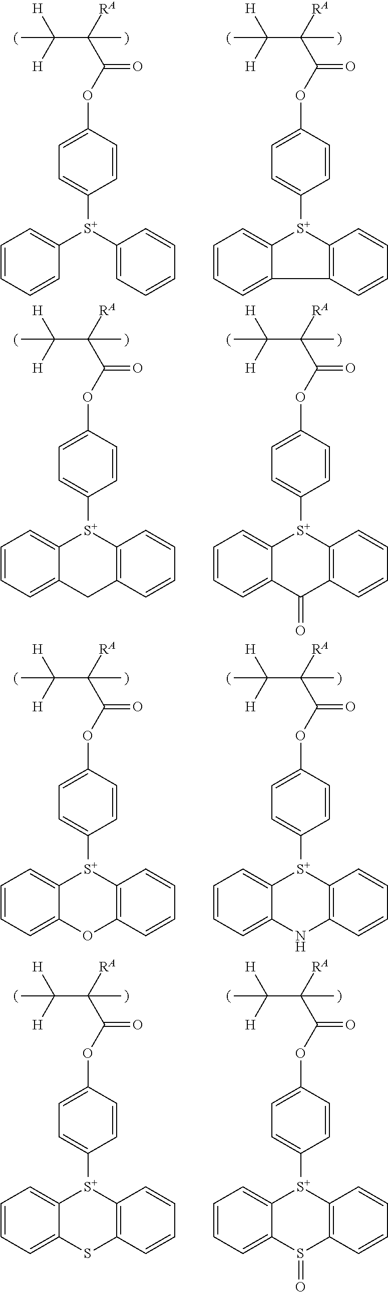

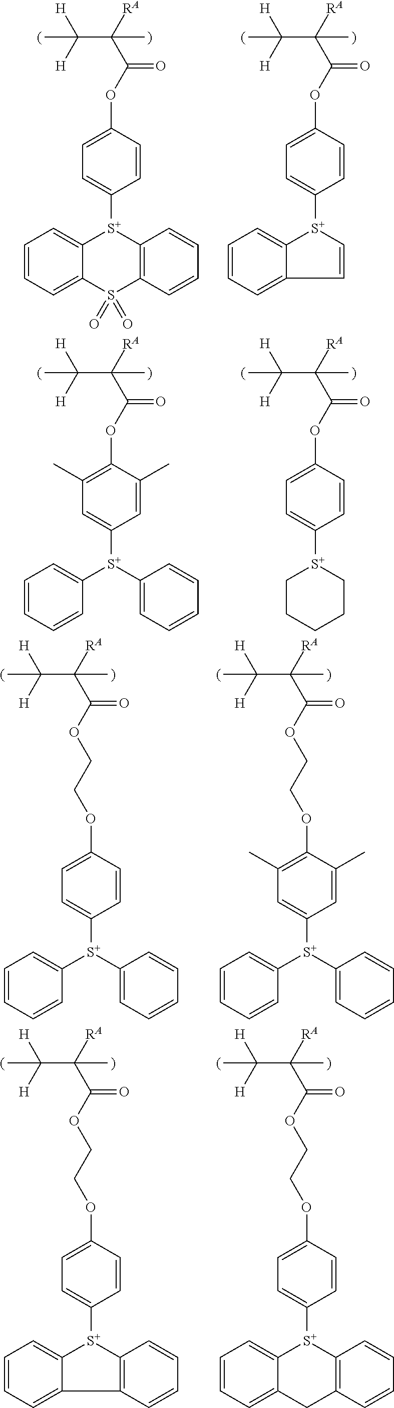

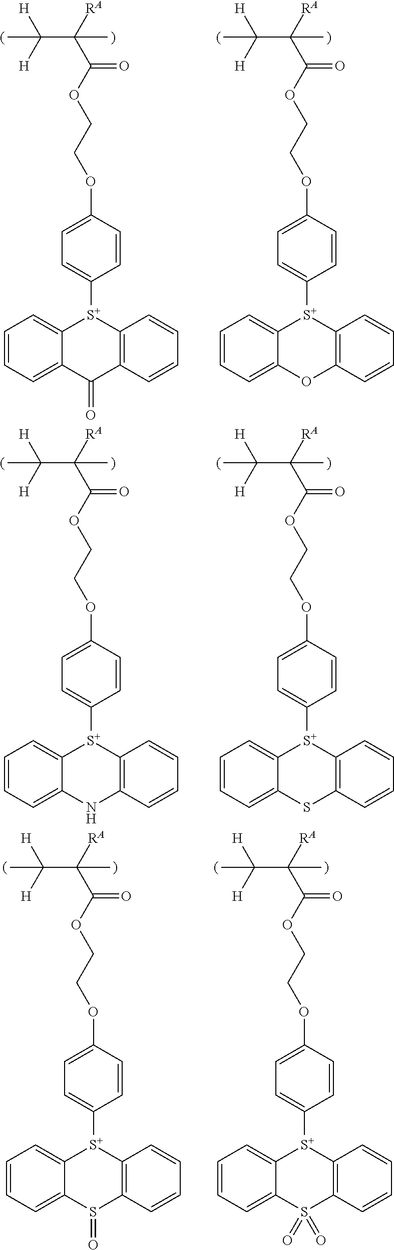

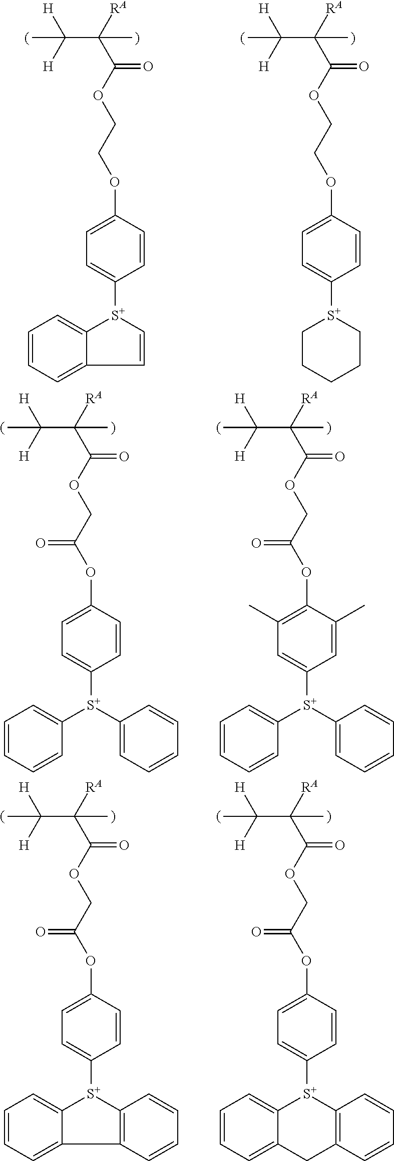

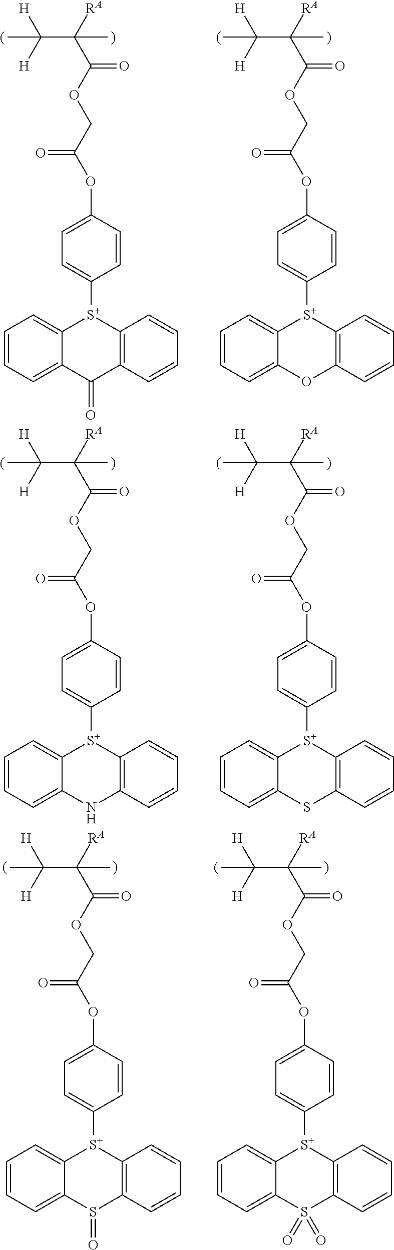

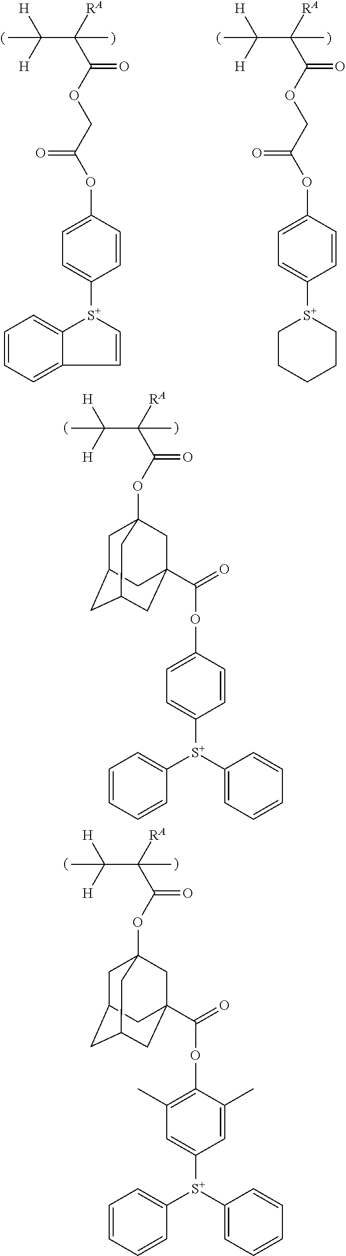

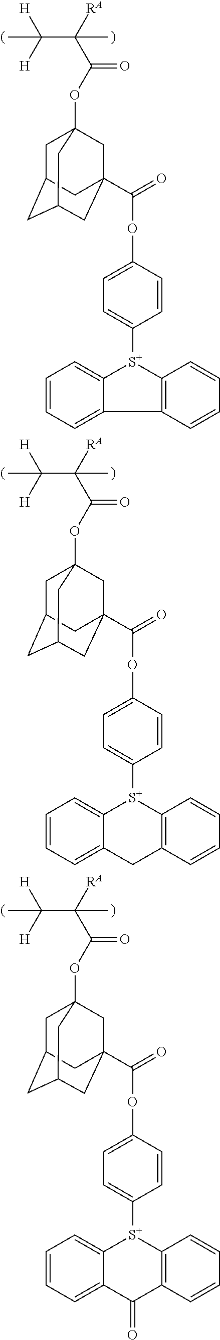

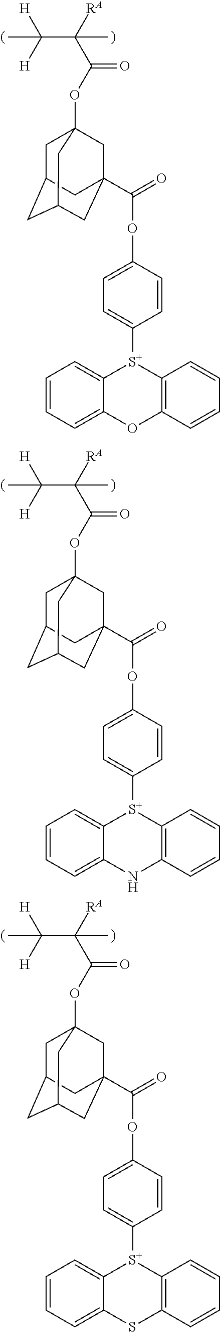

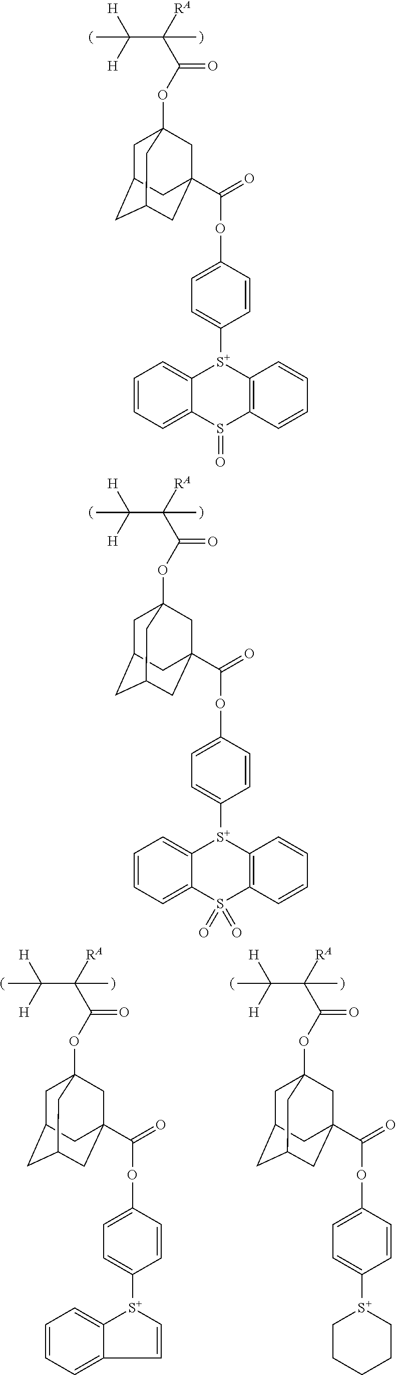

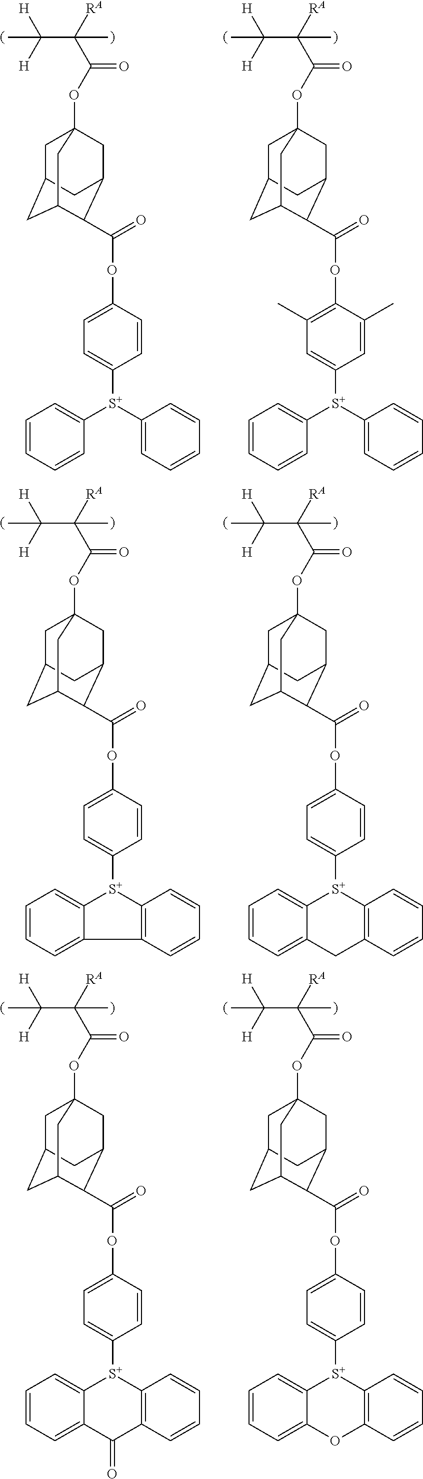

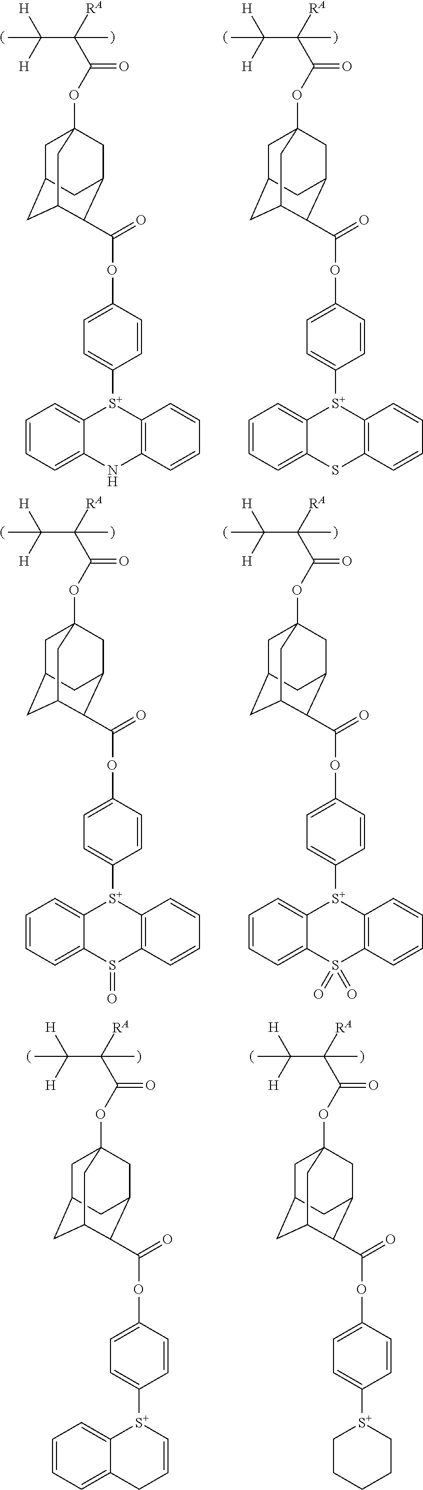

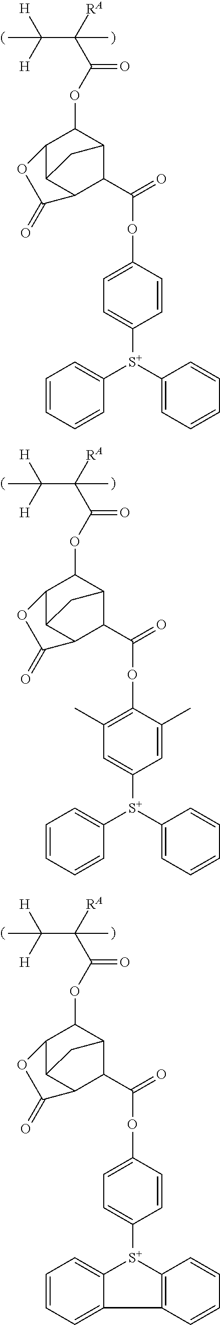

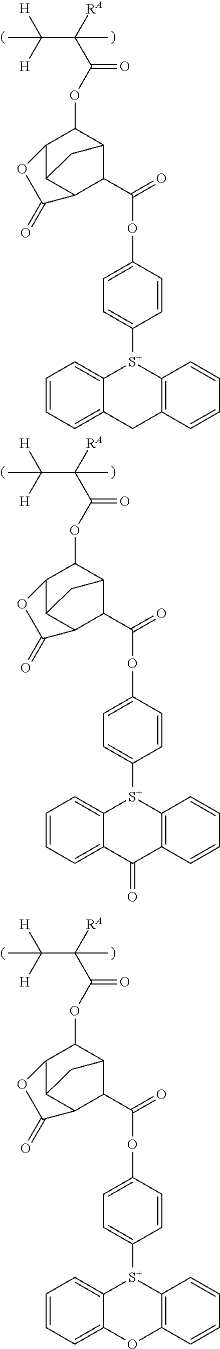

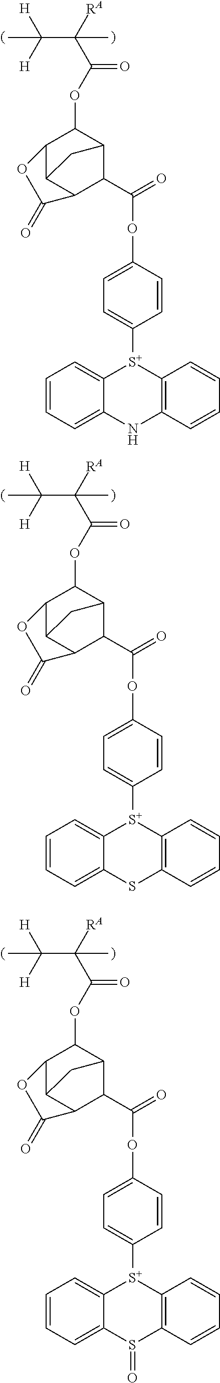

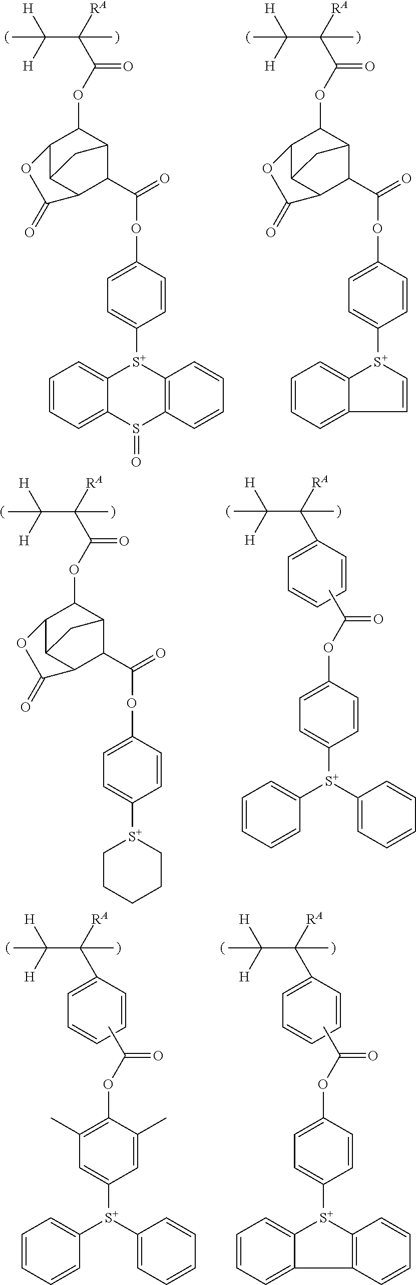

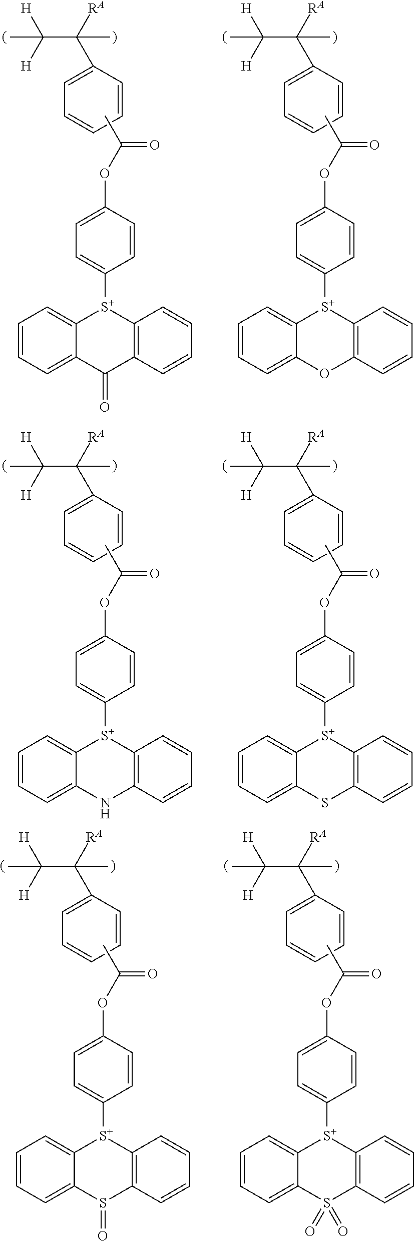

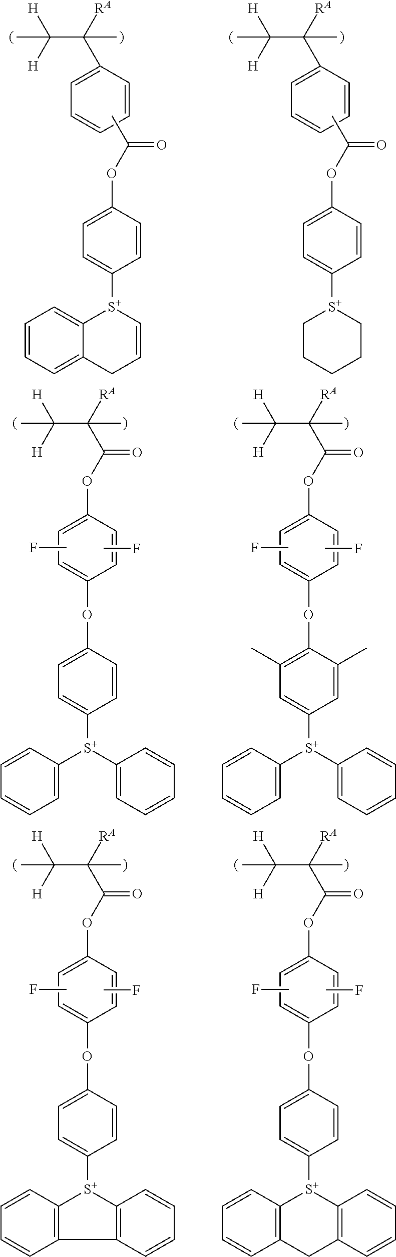

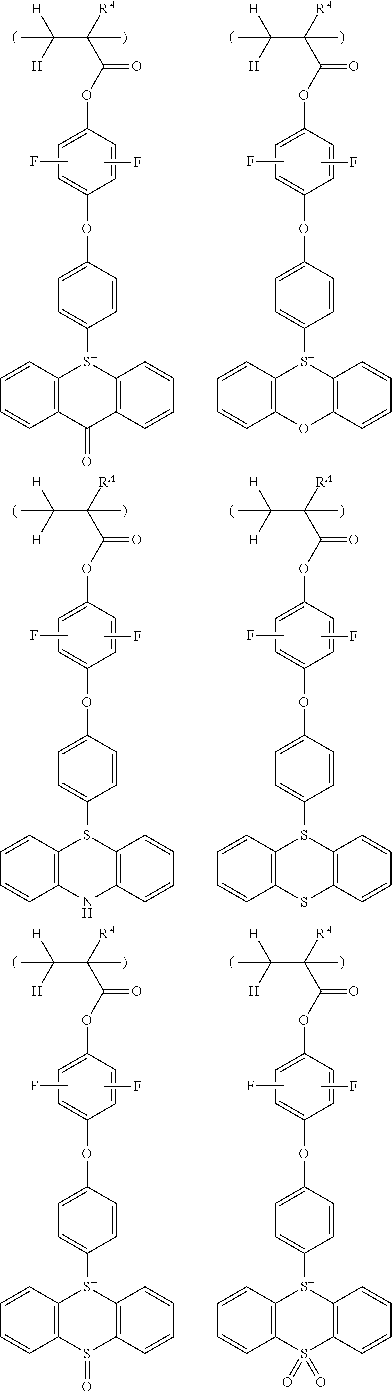

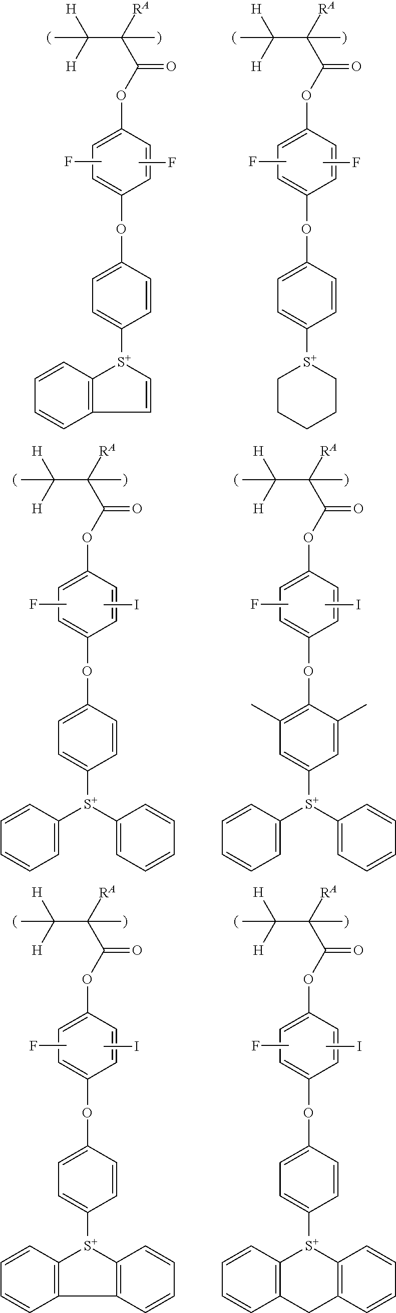

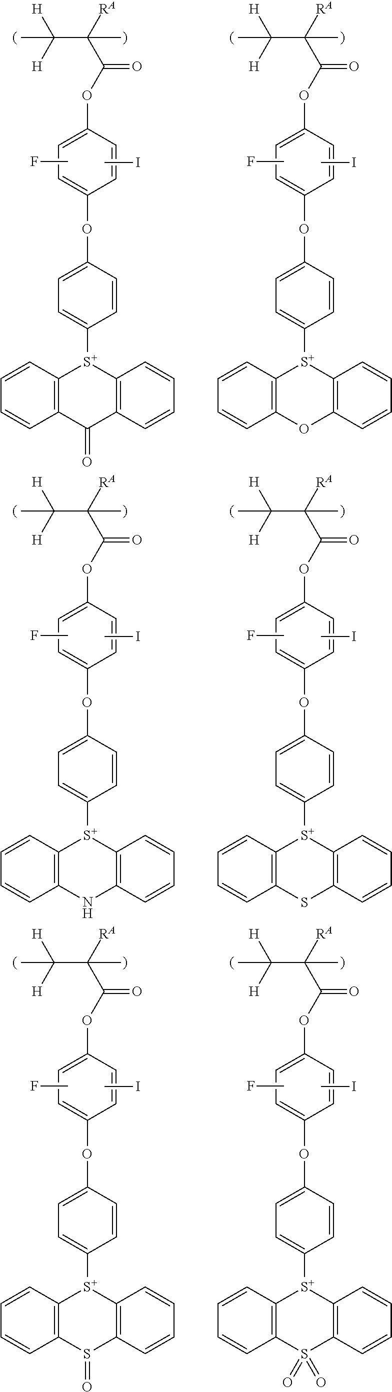

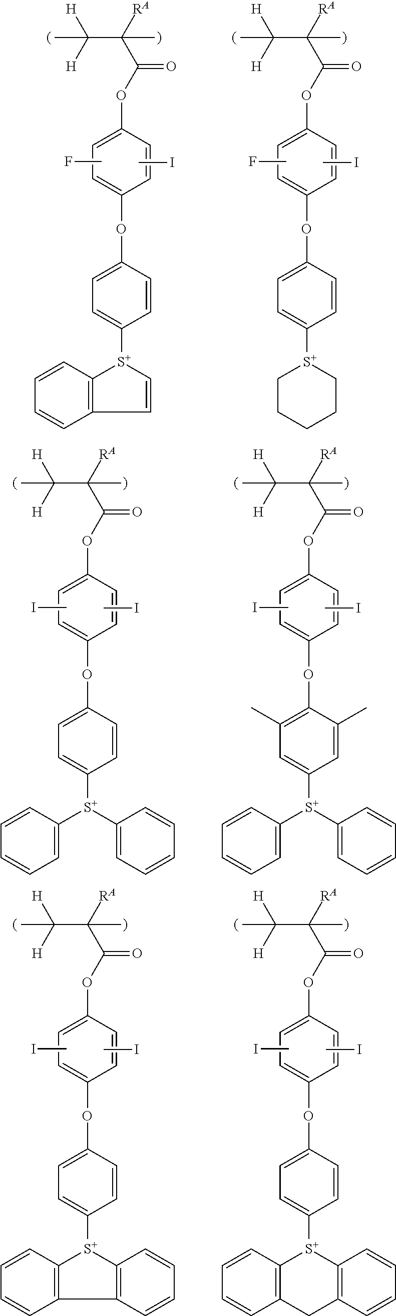

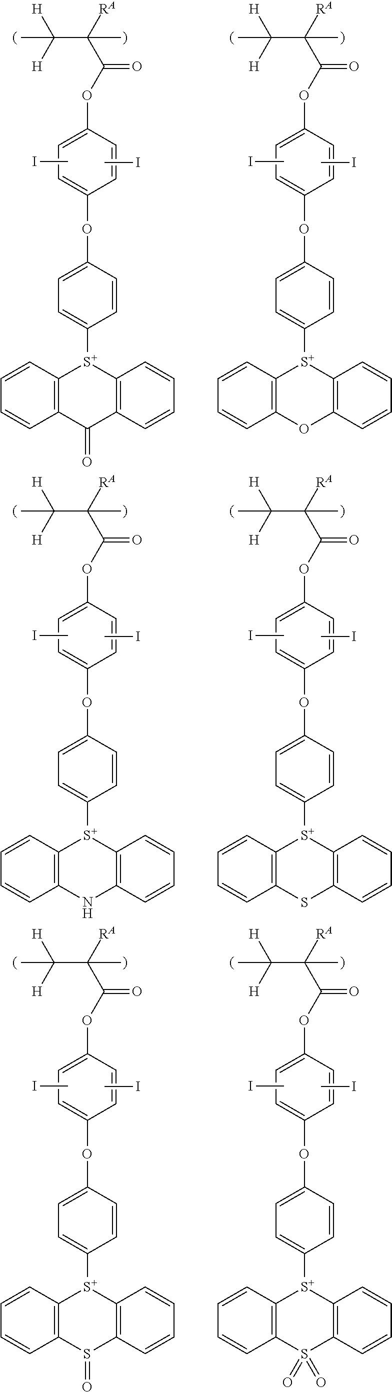

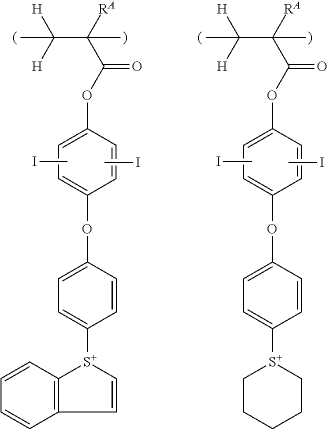

In formula (c1), M⁻ is a non-nucleophilic counter ion. Halide, sulfonate, imide and methide anions are preferred. Examples of the non-nucleophilic counter ion include halide ions such as chloride and bromide ions; sulfonate anions, specifically fluoroalkylsulfonate ions such as triflate, 1,1,1-trifluoroethanesulfonate, and nonafluorobutanesulfonate, arylsulfonate ions such as tosylate, benzenesulfonate, 4-fluorobenzenesulfonate, and 1,2,3,4,5-pentafluorobenzenesulfonate, alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; and methide ions such as tris(trifluoromethylsulfonyl)methide and tris(perfluoroethylsulfonyl)methide.

Anions having the following formulae (c1-1) to (c1-4) are also useful as the non-nucleophilic counter ion.

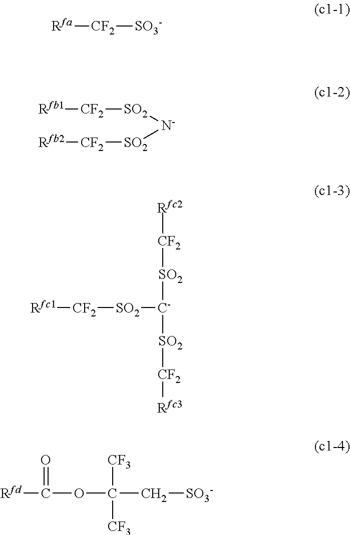

In formula (c1-1), R^{fa} is fluorine or a C₁-C₄₀ hydrocarbyl group which may contain a heteroatom. The hydrocarbyl group may be saturated or unsaturated and straight, branched or cyclic. Examples thereof are as will be exemplified below for the hydrocarbyl group R^{fa1} in formula (c1-1-1).

Of the anions of formula (c1-1), an anion having the formula (c1-1-1) is preferred.

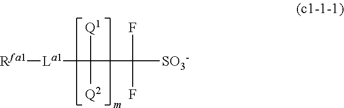

In formula (c1-1-1), Q¹ and Q² are each independently hydrogen, fluorine or a C₁-C₆ fluorinated saturated hydrocarbyl group. It is preferred for solvent solubility that at least one of Q¹ and Q² be trifluoromethyl. The subscript m is 0, 1, 2, 3 or 4, most preferably 1.

R^{fa1} is a C₁-C₃₅ hydrocarbyl group which may contain a heteroatom. As the heteroatom, oxygen, nitrogen, sulfur and halogen atoms are preferred, with oxygen being most preferred. Of the hydrocarbyl groups, those groups of 6 to 30 carbon atoms are preferred from the aspect of achieving a high resolution in forming patterns of small feature size. The hydrocarbyl group may be saturated or unsaturated and straight, branched or cyclic. Examples thereof include C₁-C₃₅ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, and icosyl; C₃-C₃₅ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclododecyl, tetracyclododecylmethyl, and dicyclohexylmethyl; C₂-C₃₅ unsaturated aliphatic hydrocarbyl groups such as 2-propenyl and 3-cyclohexenyl; C₆-C₃₅ aryl groups such as phenyl, 1-naphthyl, 2-naphthyl and 9-fluorenyl; and C₇-C₃₅ aralkyl groups such as benzyl and diphenylmethyl, and combinations thereof.

In the foregoing hydrocarbyl groups, some or all hydrogen may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl.

In formula (c1-1-1), L^{a1} is a single bond, ether bond, ester bond, sulfonate ester bond, carbonate bond or carbamate bond. From the aspect of synthesis, an ether bond or ester bond is preferred, with the ester bond being more preferred.

Examples of the anion having formula (c1-1) are shown below, but not limited thereto. Herein Q¹ is as defined above.

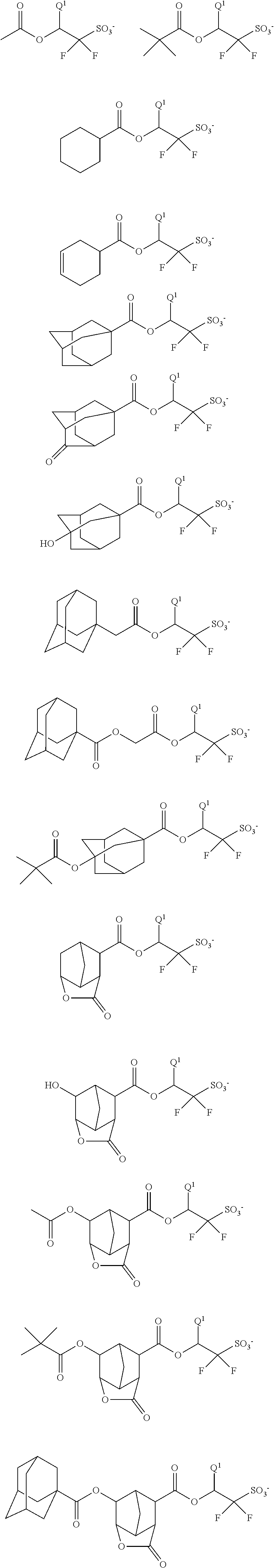

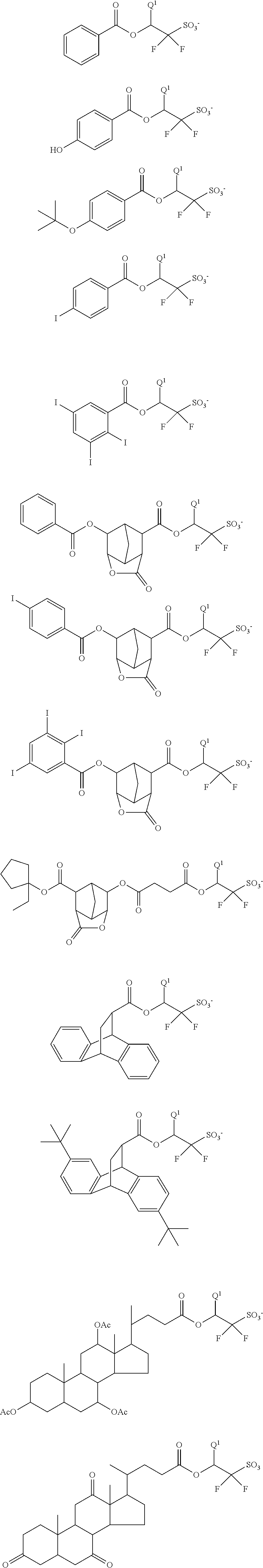

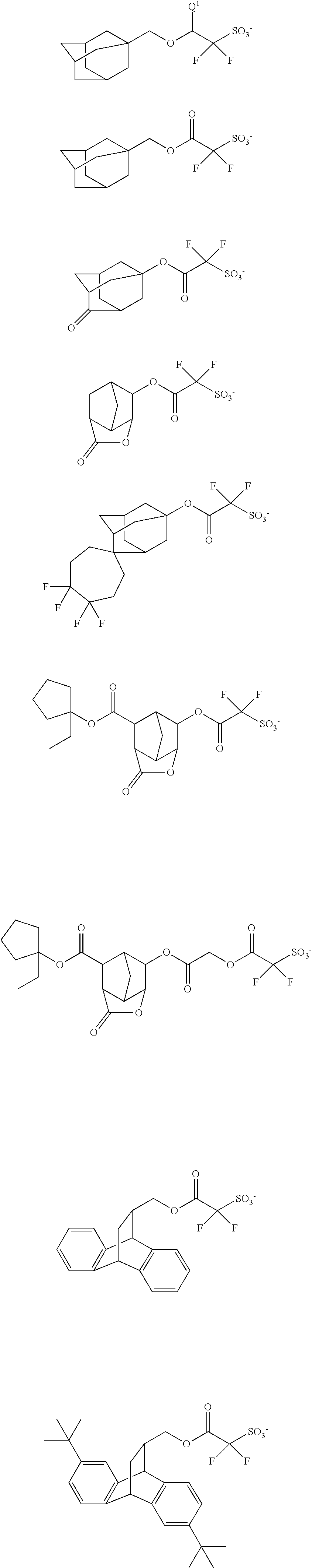

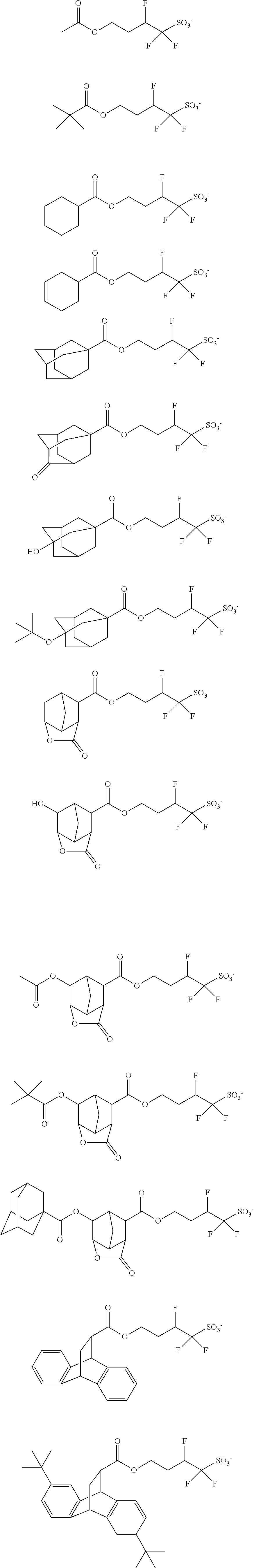

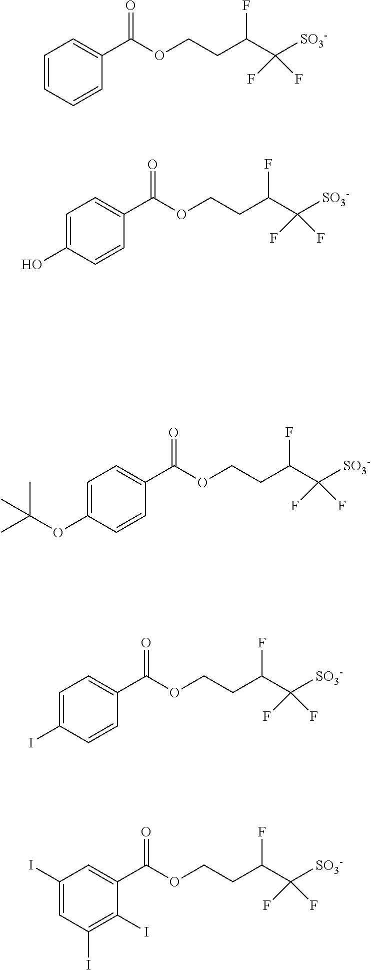

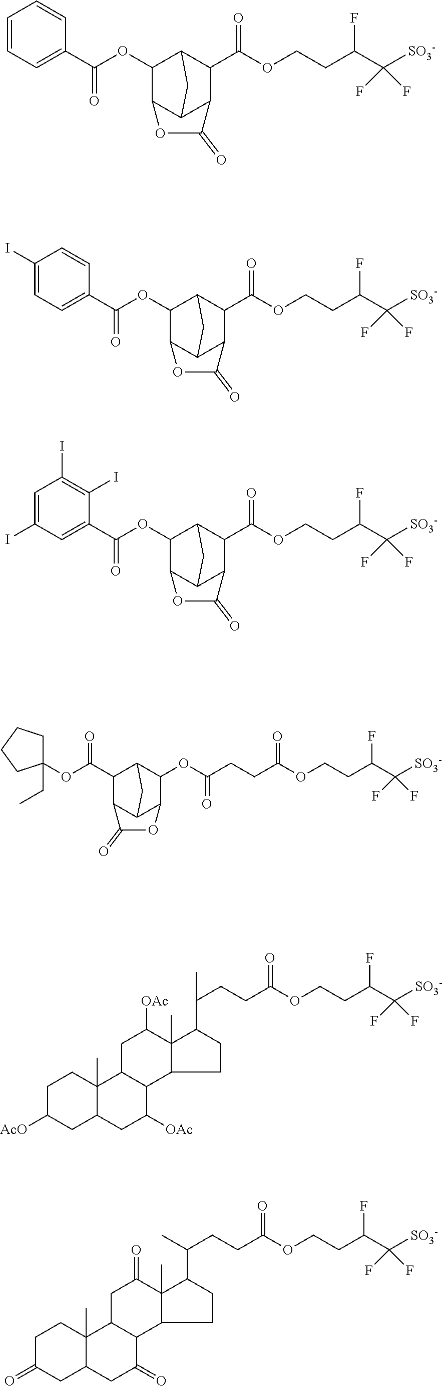

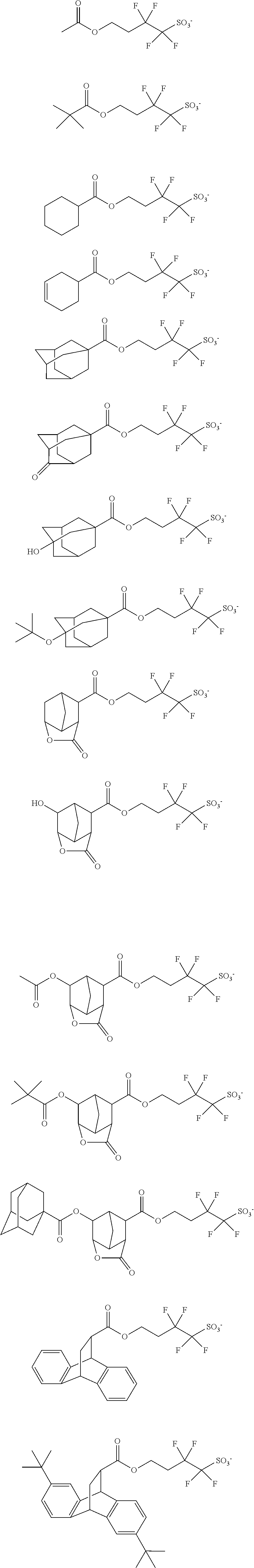

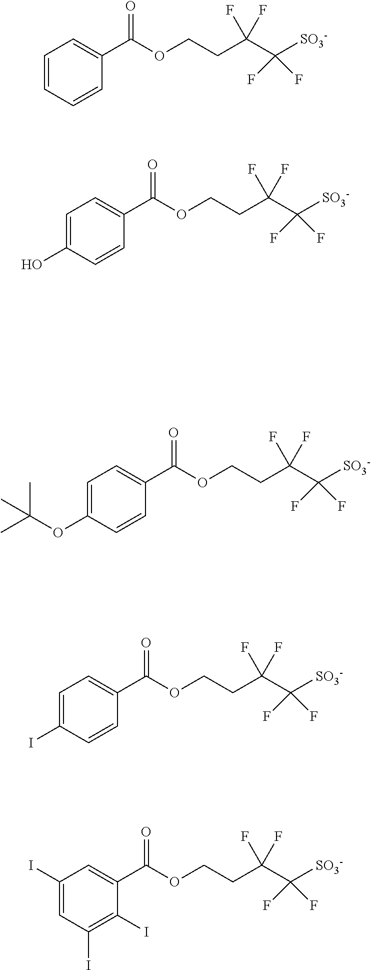

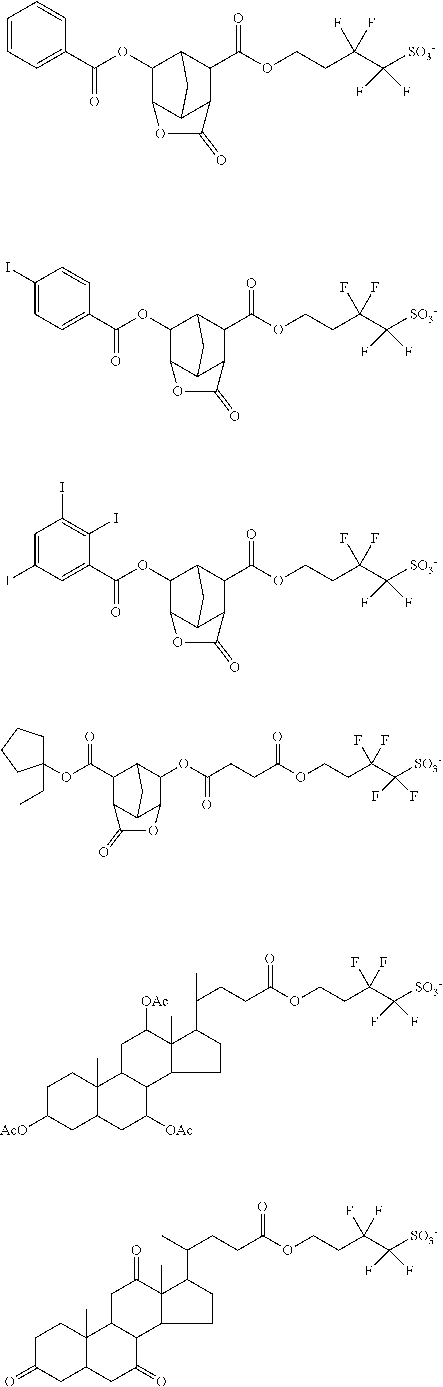

In formula (c1-2), R^{fb1} and R^{fb2} are each independently fluorine or a C₁-C₄₀ hydrocarbyl group which may contain a heteroatom. The hydrocarbyl group may be saturated or unsaturated and straight, branched or cyclic. Examples thereof are as exemplified above for the hydrocarbyl group R^{fa1} in formula (c1-1-1). Preferably R^{fb1} and R^{fb2} are fluorine or C₁-C₄ straight fluorinated alkyl groups. Also, R^{fb1} and R^{fb2} may bond together to form a ring with the linkage: —CF₂—SO₂—N—SO₂—CF₂— to which they are attached. It is preferred that a combination of R^{fb1} and R^{fb2} be a fluorinated ethylene or fluorinated propylene group.

In formula (c1-3), R^{fc1}, R^{fc2} and R^{fc3} are each independently fluorine or a C₁-C₄₀ hydrocarbyl group which may contain a heteroatom. The hydrocarbyl group may be saturated or unsaturated and straight, branched or cyclic. Examples thereof are as exemplified above for the hydrocarbyl group R^{fa1} in formula (c1-1-1). Preferably R^{fc1}, R^{fc2} and R^{fc3} are fluorine or C₁-C₄ straight fluorinated alkyl groups. Also, R^{fc1} and R^{fc2} may bond together to form a ring with the linkage: —CF₂—SO₂—C⁻—SO₂—CF₂— to which they are attached. It is preferred that a combination of R^{fc1} and R^{fc2} be a fluorinated ethylene or fluorinated propylene group.

In formula (c1-4), R^{fd} is a C₁-C₄₀ hydrocarbyl group which may contain a heteroatom. The hydrocarbyl group may be saturated or unsaturated and straight, branched or cyclic. Examples thereof are as exemplified above for R^{fa1}.

Examples of the anion having formula (c1-4) are shown below, but not limited thereto.

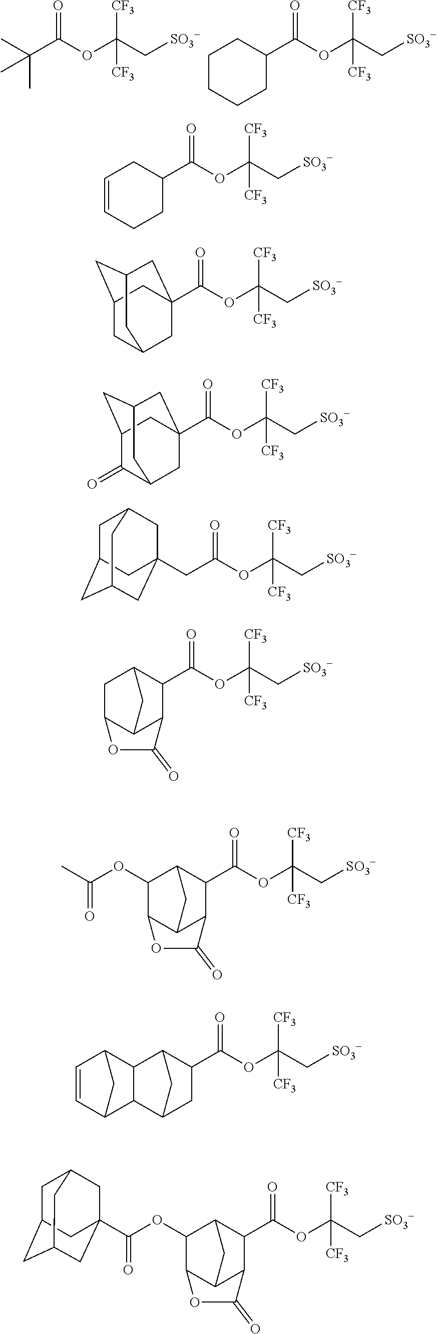

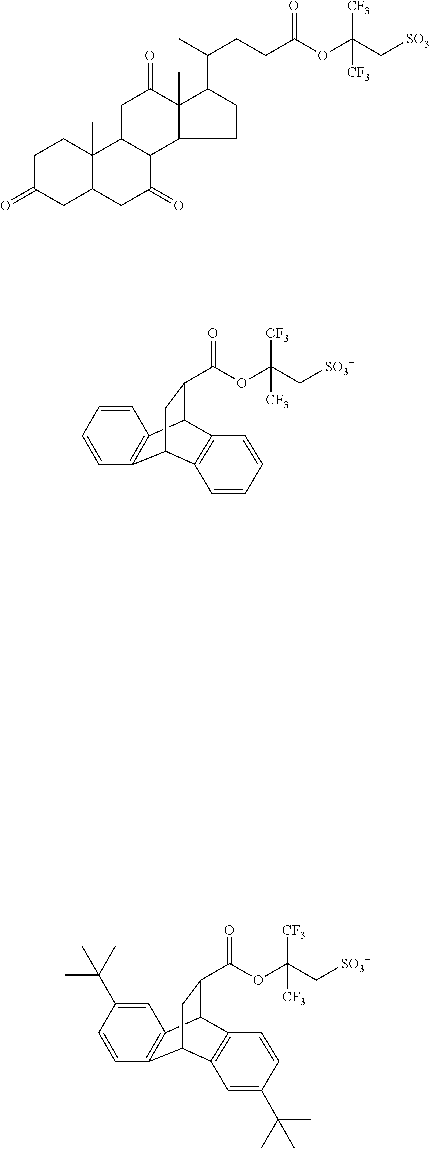

Anions having an iodized or brominated aromatic ring are also useful as the non-nucleophilic counter ion. These anions have the formula (c1-5).

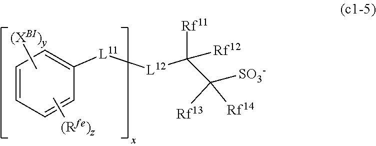

In formula (c1-5), x is 1, 2 or 3, y is 1, 2, 3, 4 or 5, z is 0, 1, 2 or 3, and y+z is from 1 to 5. Preferably, y is 1, 2 or 3, more preferably 2 or 3, and z is 0, 1 or 2.

X^{BI} is iodine or bromine. A plurality of X^{BI} may be identical or different when x and/or y is 2 or more.

L¹¹ is a single bond, ether bond, ester bond, or a C₁-C₆ saturated hydrocarbylene group which may contain an ether bond or ester bond. The saturated hydrocarbylene group may be straight, branched or cyclic.

L¹² is a single bond or a C₁-C₂₀ divalent linking group when x=1, or a C₁-C₂₀ (x+1)-valent linking group when x=2 or 3. The linking group may contain an oxygen, sulfur or nitrogen atom.

R^{fe} is hydroxy, carboxy, fluorine, chlorine, bromine, amino group, or a C₁-C₂₀ hydrocarbyl, C₁-C₂₀ hydrocarbyloxy, C₂-C₂₀ hydrocarbylcarbonyl, C₂-C₂₀ hydrocarbyloxycarbonyl, C₂-C₂₀ hydrocarbylcarbonyloxy, or C₁-C₂₀ hydrocarbylsulfonyloxy group, which may contain fluorine, chlorine, bromine, hydroxy, amino or ether bond, or —N(R^{feA})(R^{feB}), —N(R^{feC})—C(═O)—R^{feD} or —N(R^{feC})—C(═O)—O—R^{feD}. R^{feA} and R^{feB} are each independently hydrogen or a C₁-C₆ saturated hydrocarbyl group. R^{feC} is hydrogen, or a C₁-C₆ saturated hydrocarbyl group which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. R^{feD} is a C₁-C₁₆ aliphatic hydrocarbyl group, C₆-C₁₂ aryl group or C₇-C₁₅ aralkyl group, which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy, and hydrocarbylsulfonyloxy groups may be straight, branched or cyclic. A plurality of R^{fe} may be identical or different when x and/or z is 2 or more.

Of these, R^{fe} is preferably hydroxy, —N(R^{feC})—C(═O)—R^{feD}, —N(R^{feC})—C(═O)—O—R^{feD}, fluorine, chlorine, bromine, methyl or methoxy.

Rf¹¹ to Rf¹⁴ are each independently hydrogen, fluorine or trifluoromethyl, at least one of Rf¹¹ to Rf¹⁴ is fluorine or trifluoromethyl. Rf¹¹ and Rf¹², taken together, may form a carbonyl group. More preferably, both Rf¹³ and Rf¹⁴ are fluorine.

Examples of the anion having formula (c1-5) are shown below, but not limited thereto. X^{BI} is as defined above.

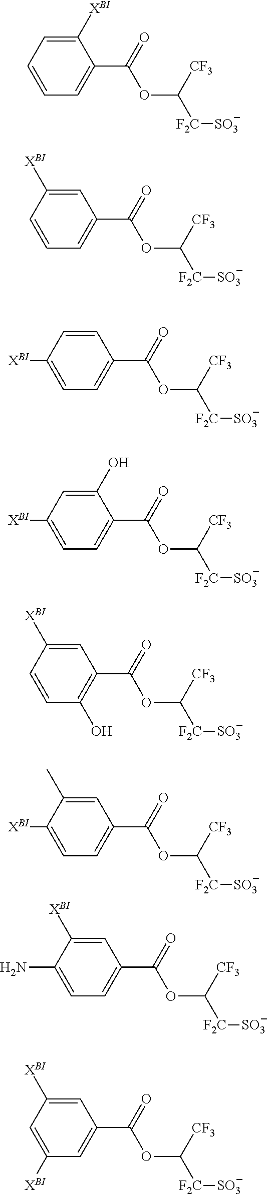

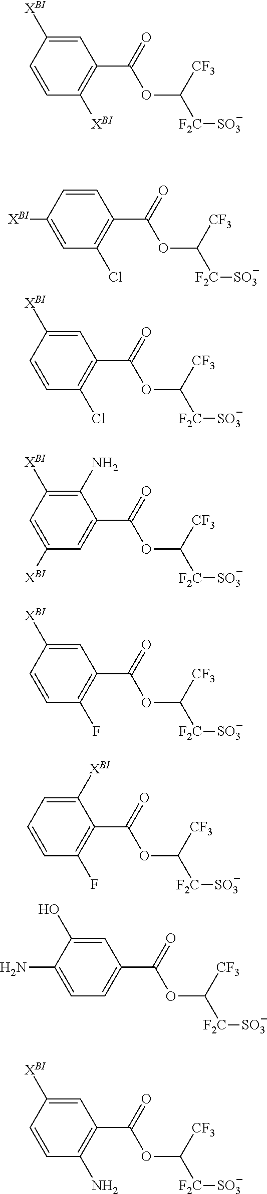

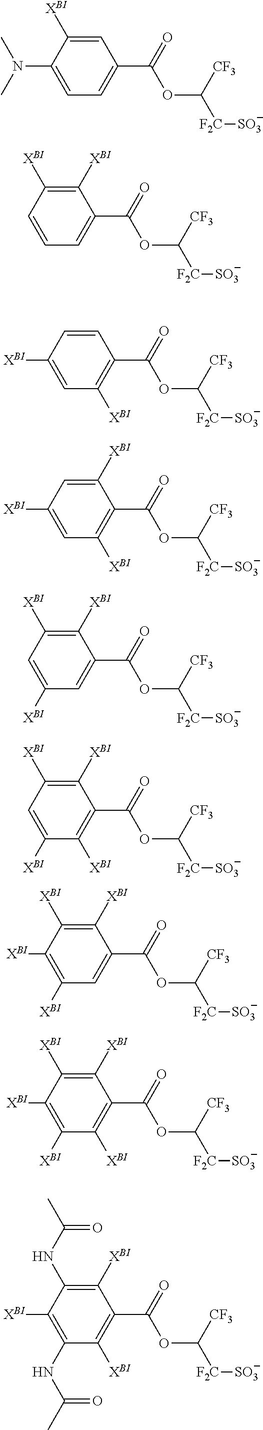

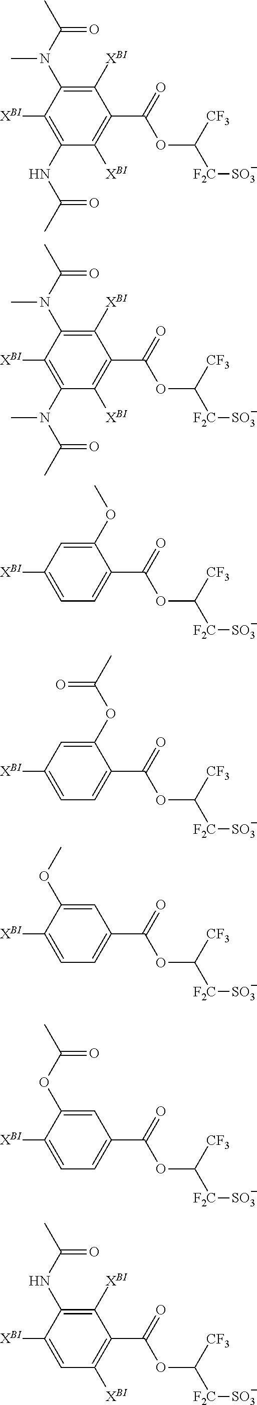

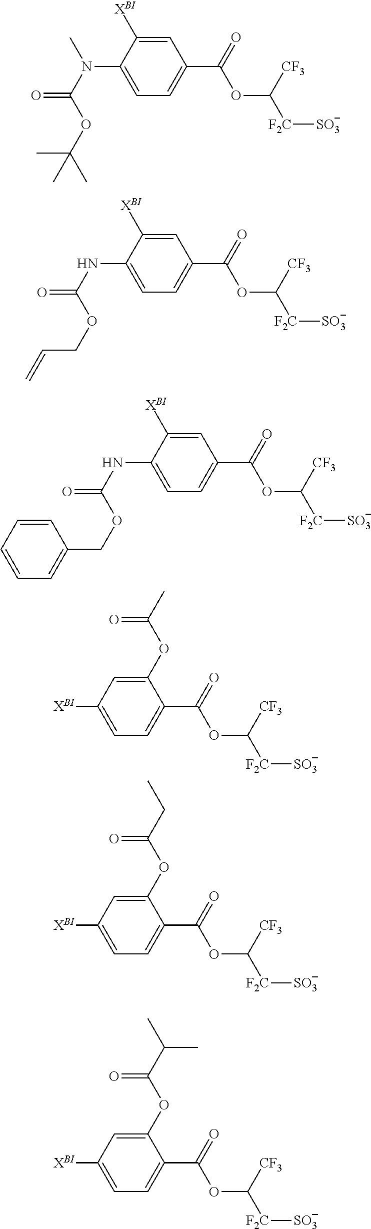

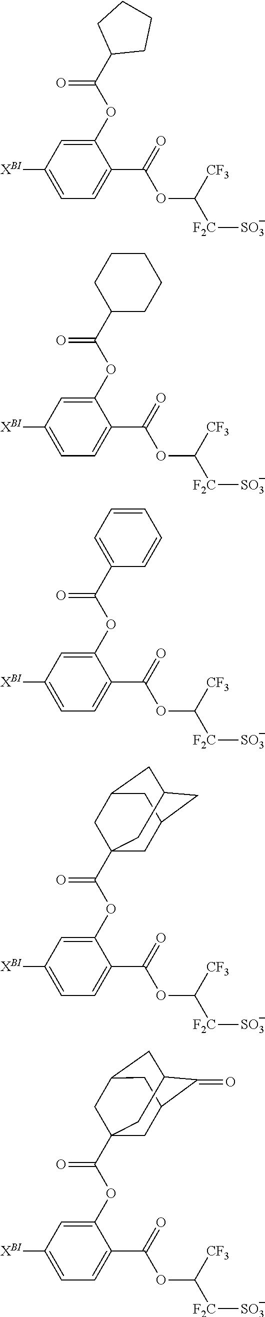

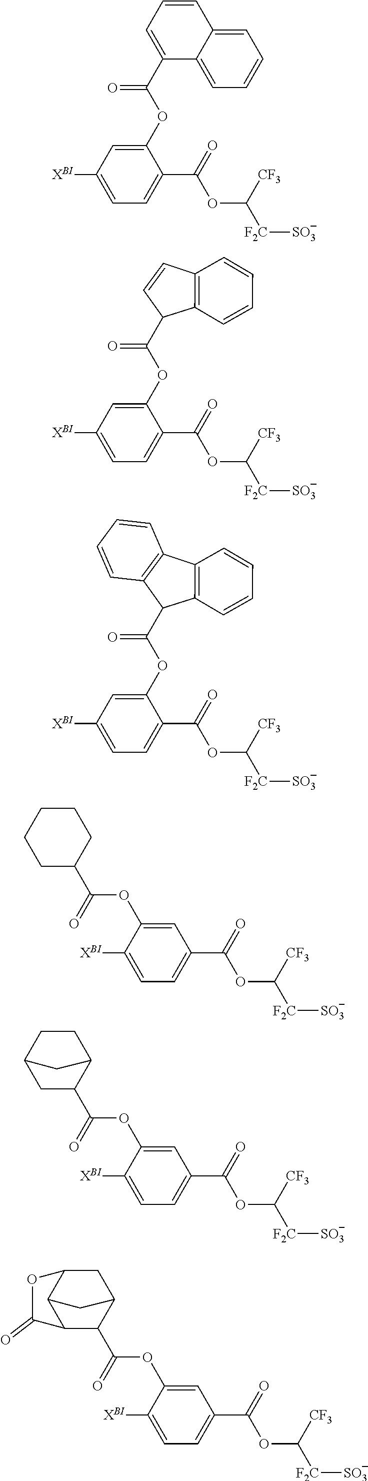

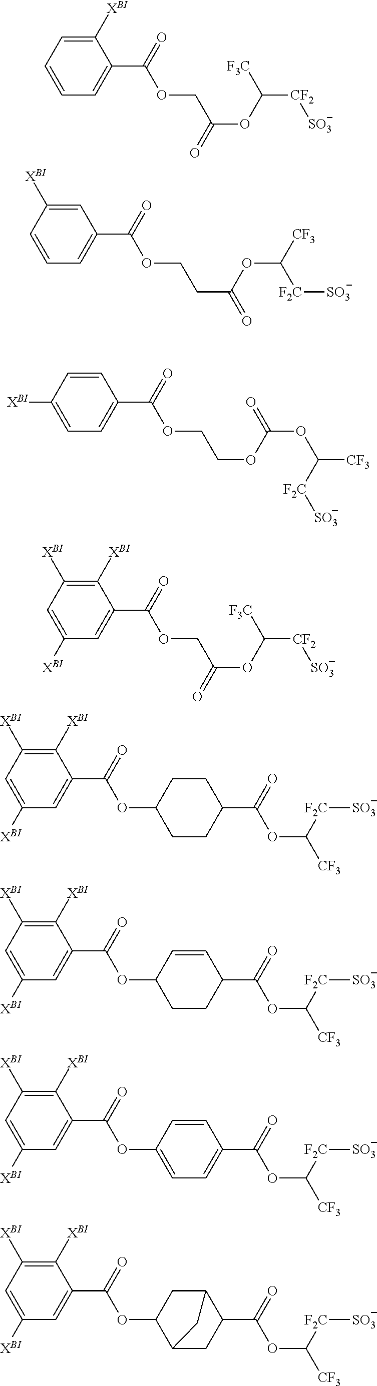

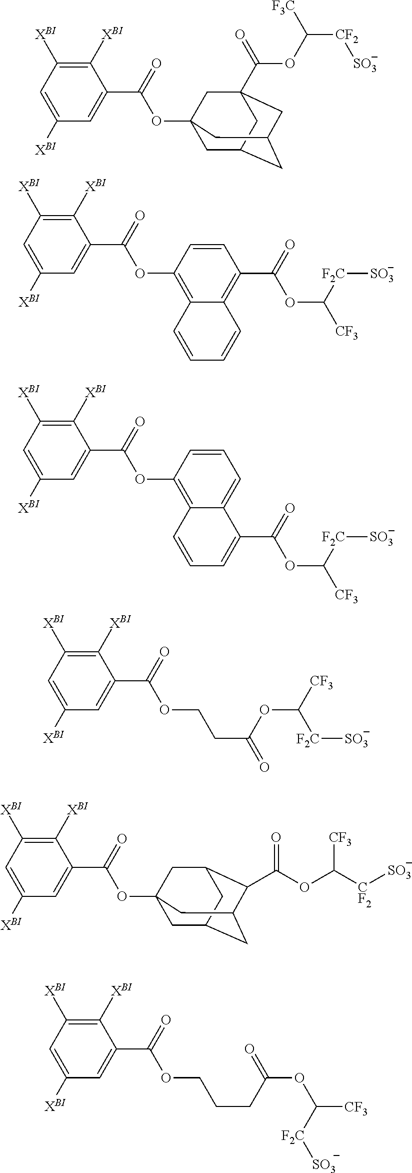

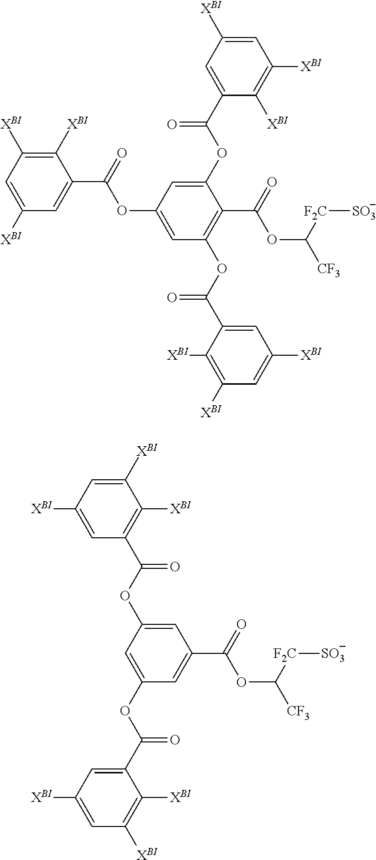

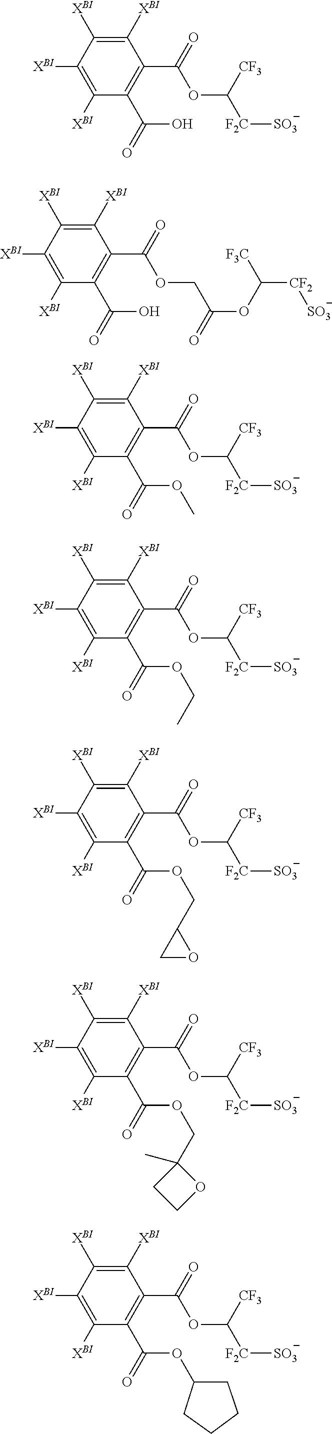

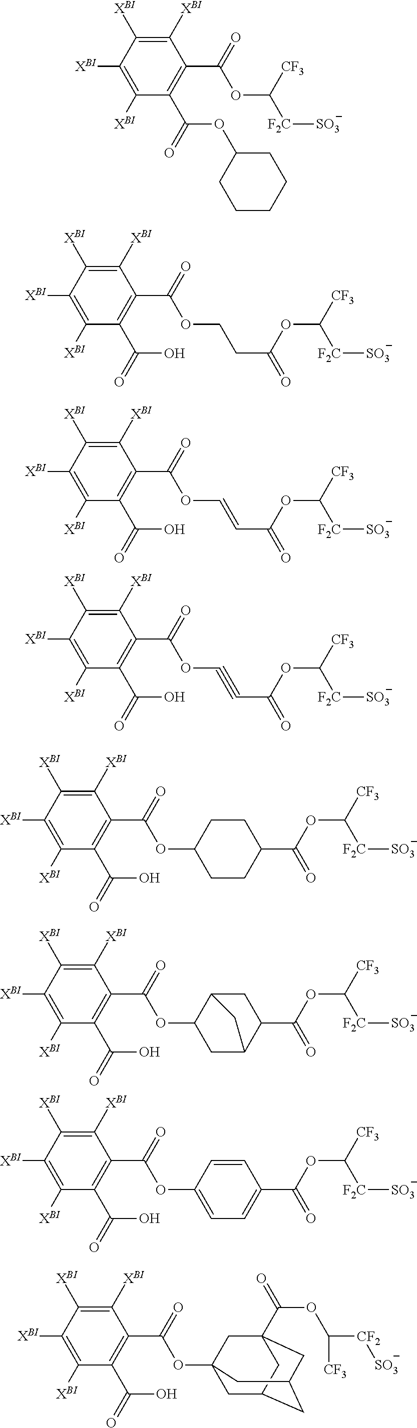

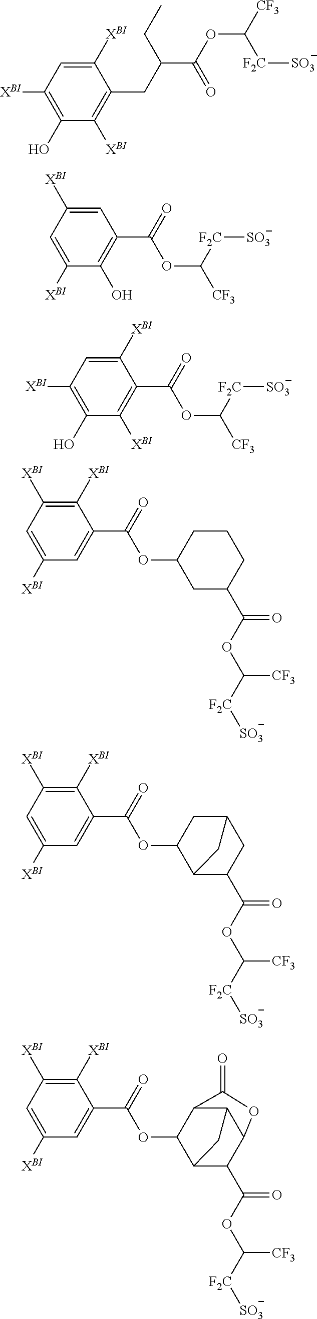

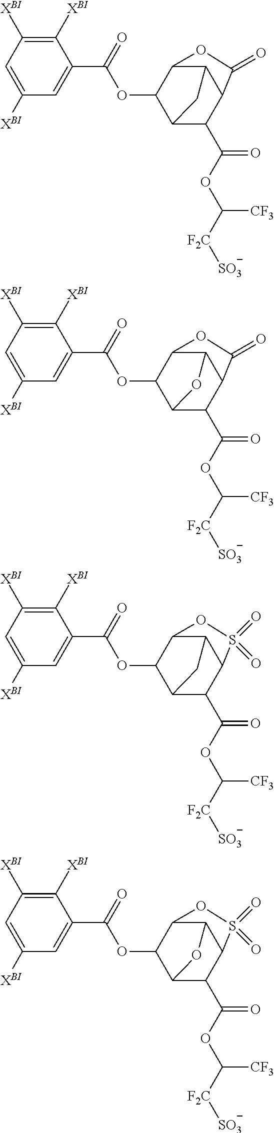

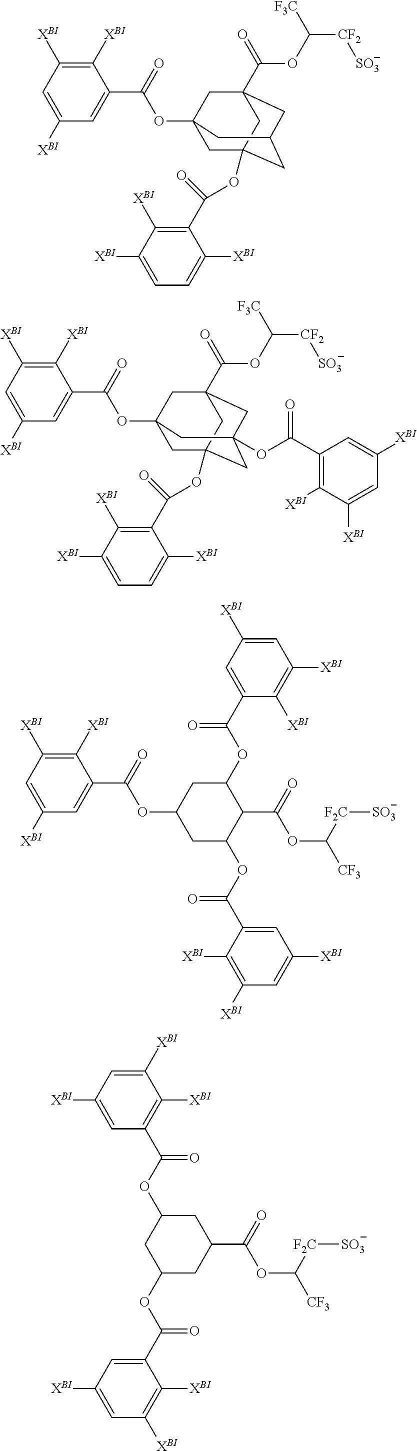

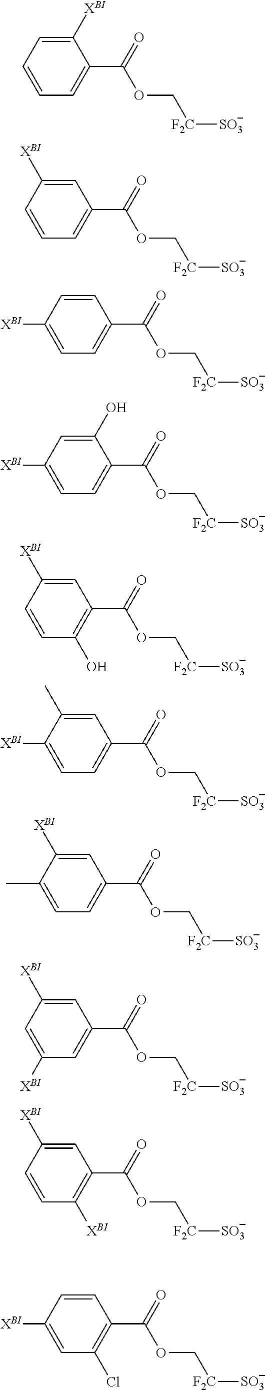

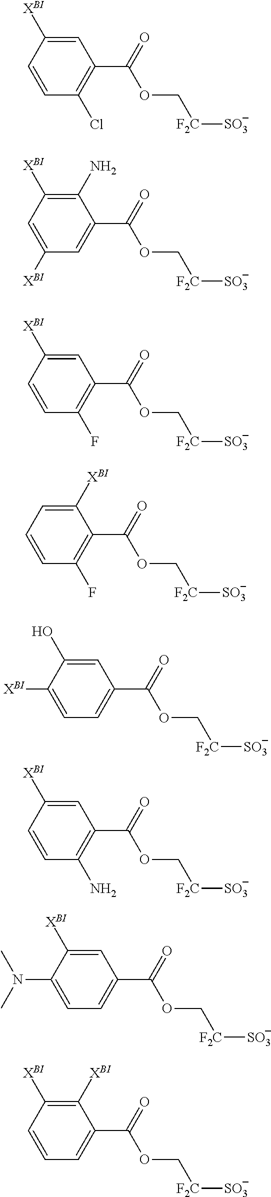

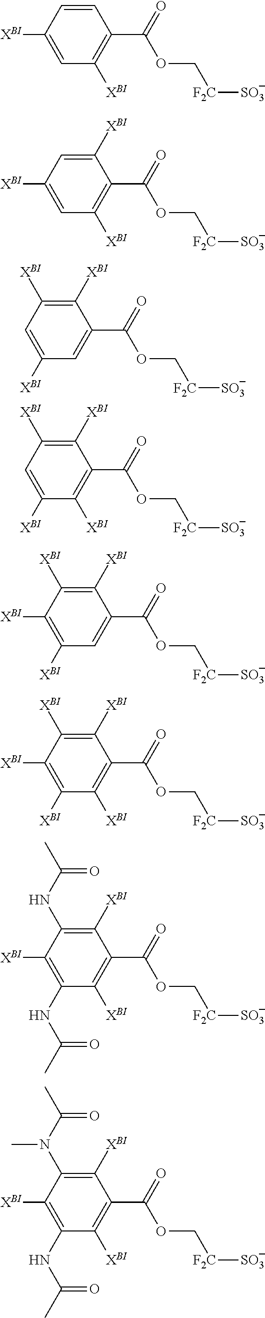

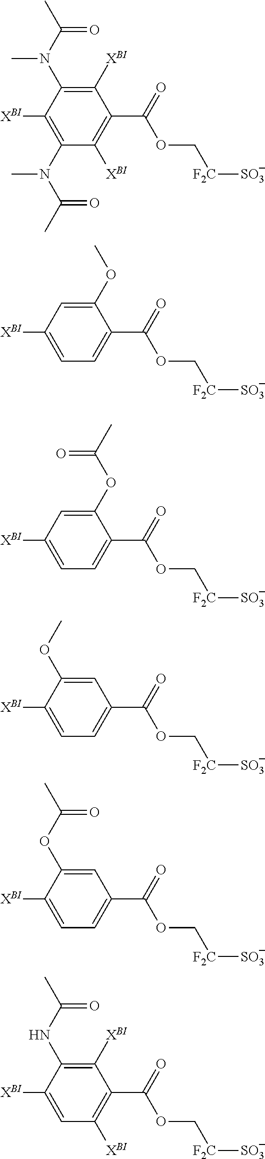

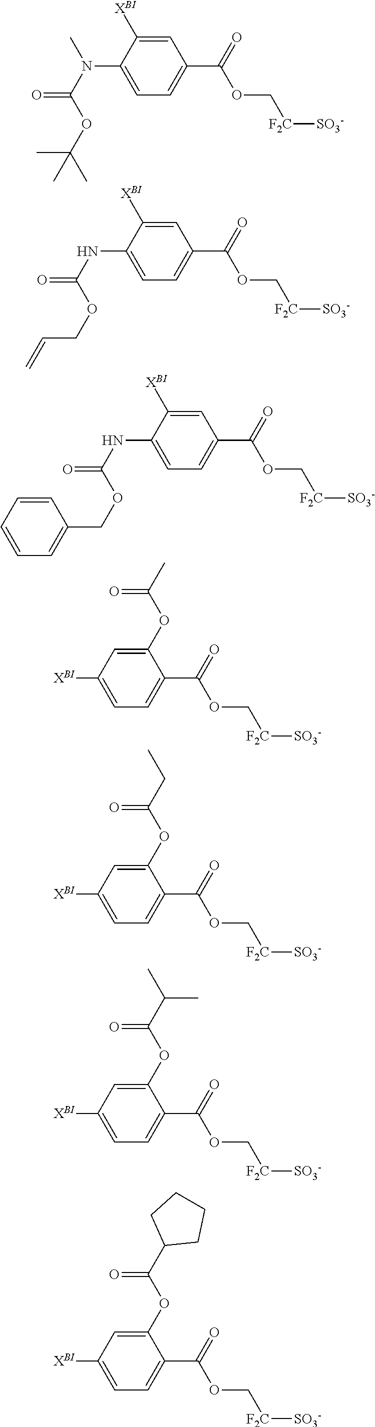

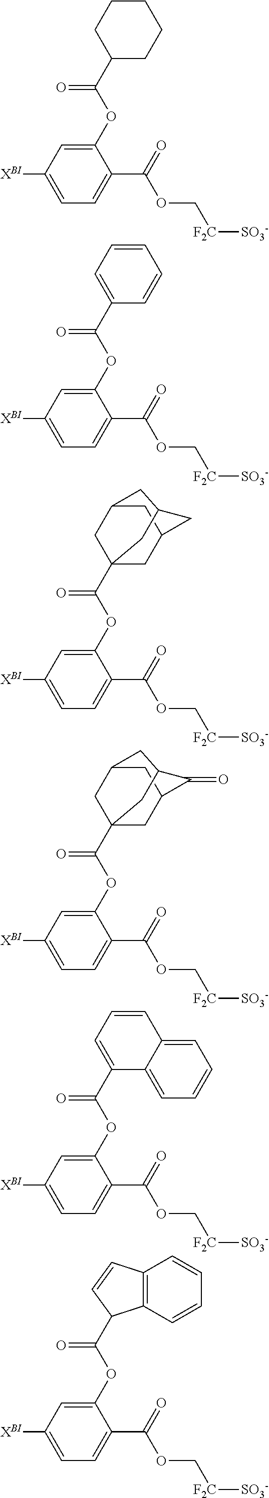

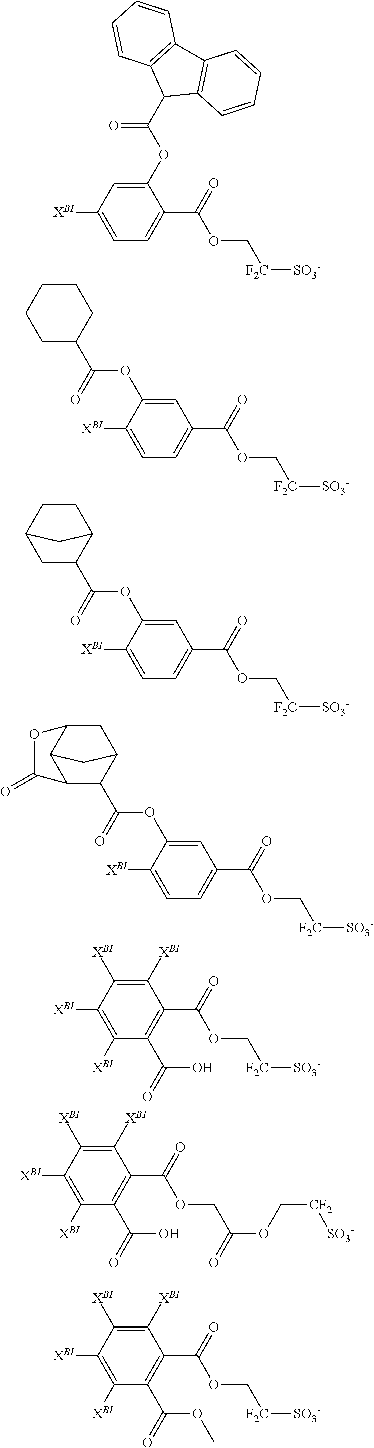

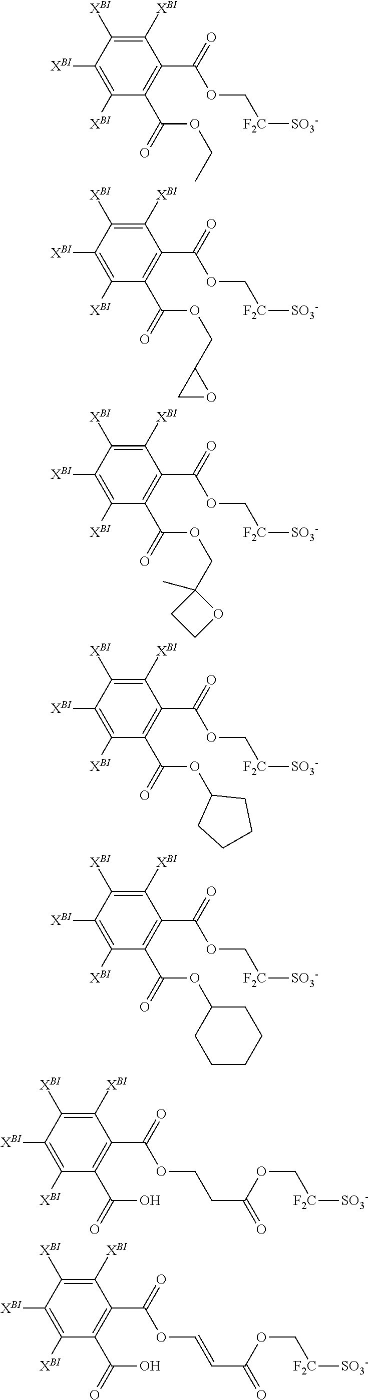

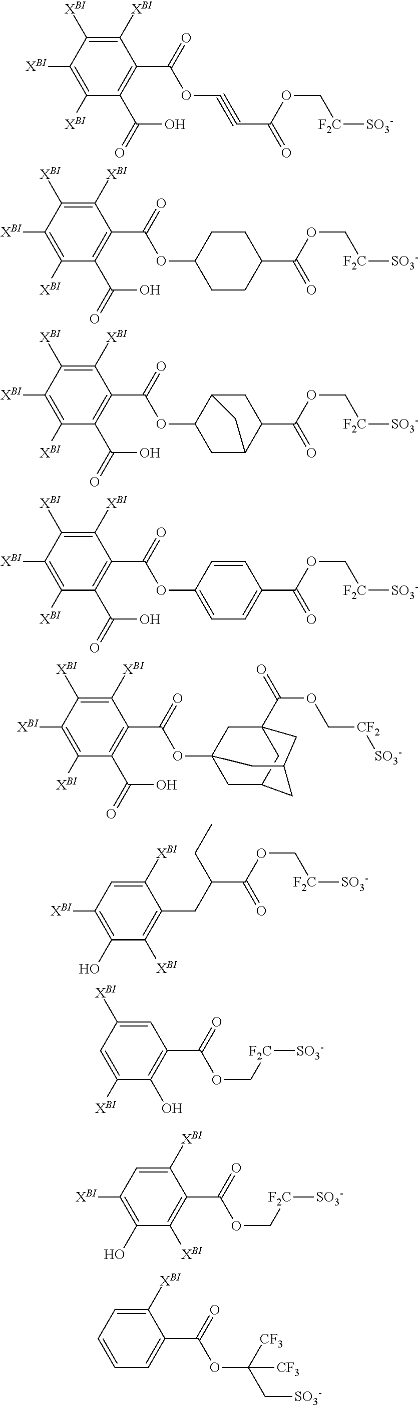

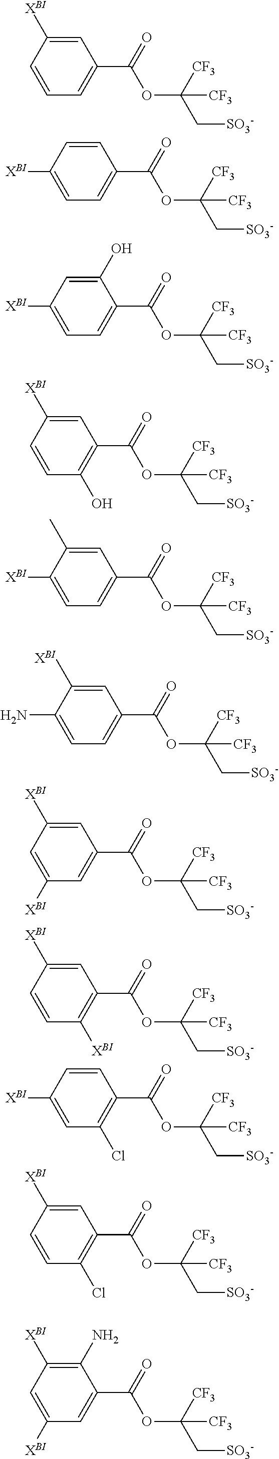

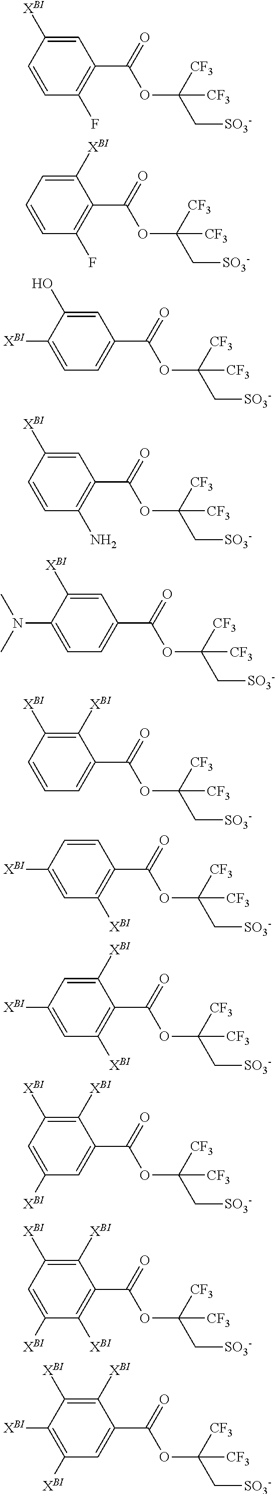

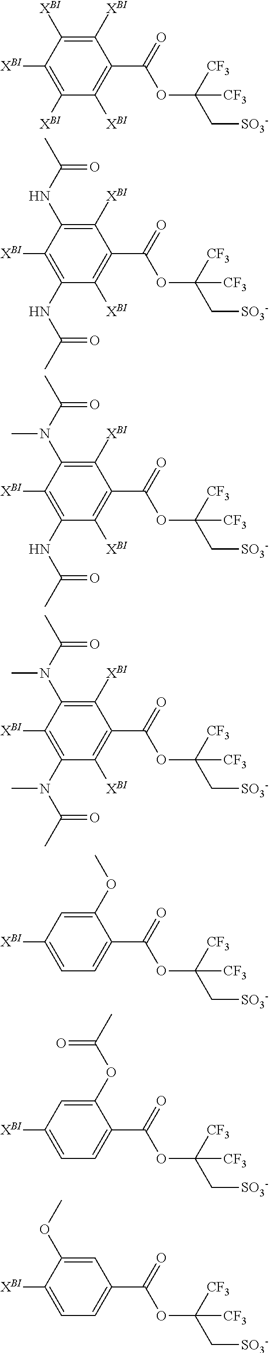

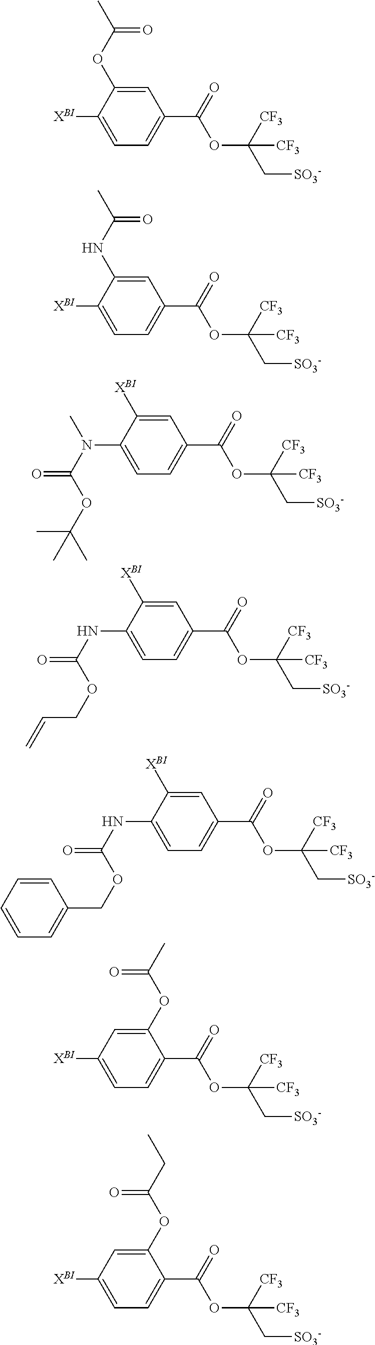

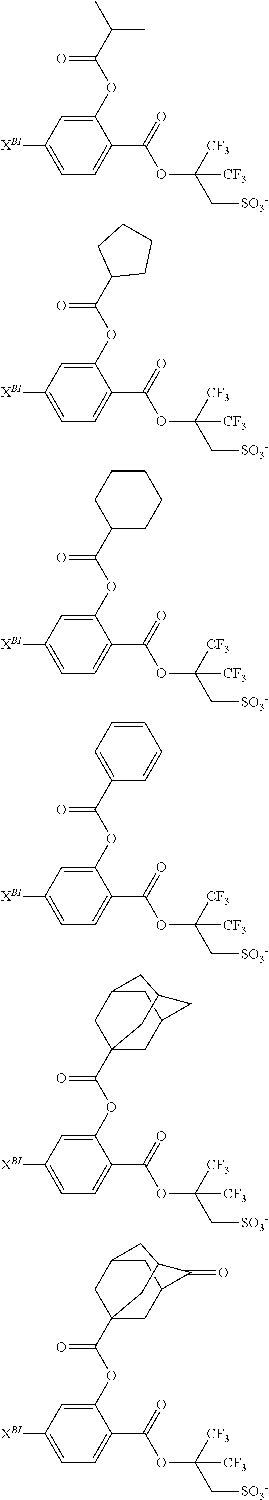

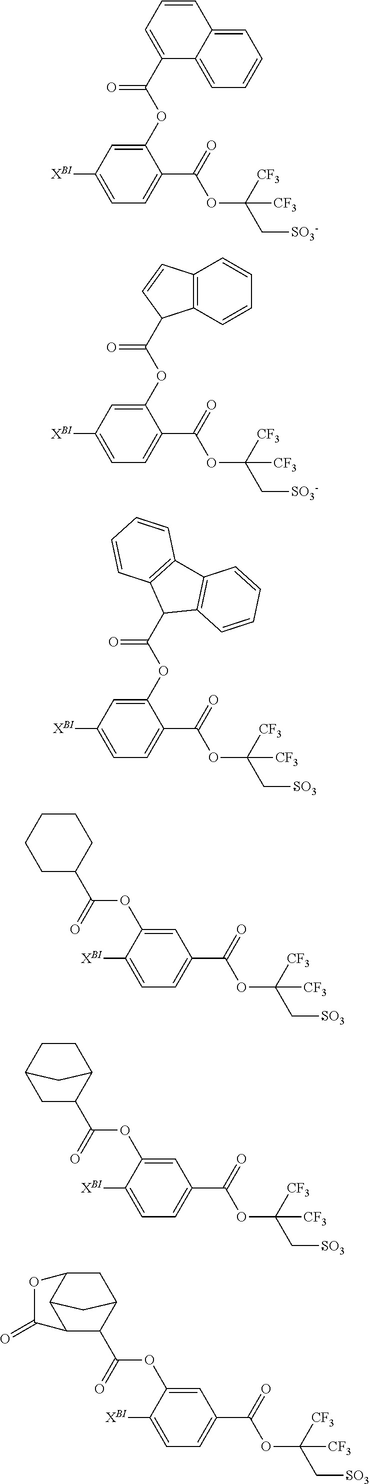

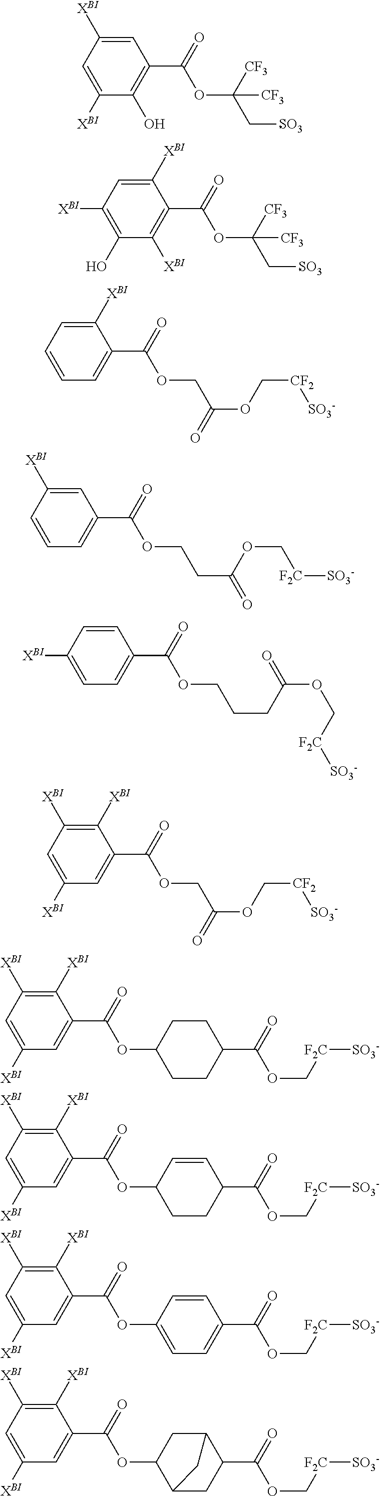

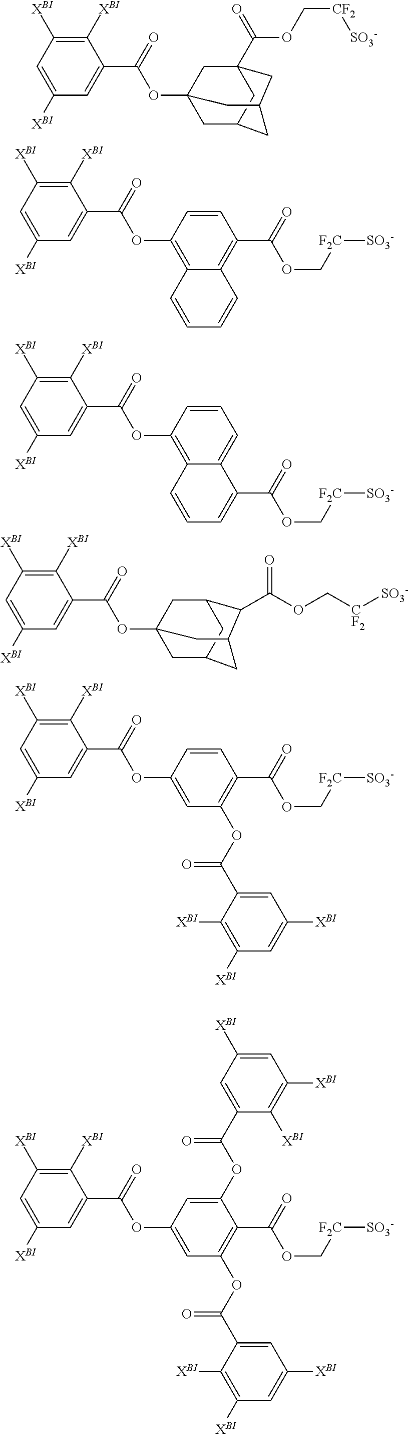

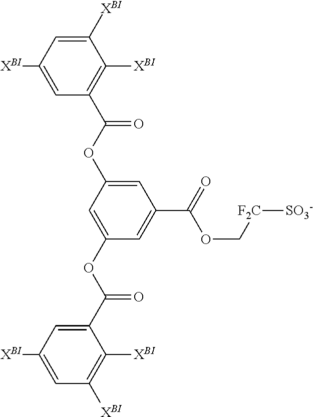

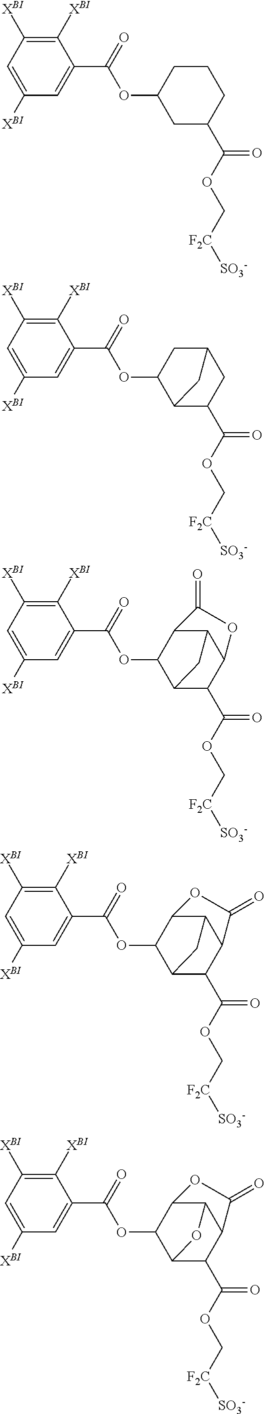

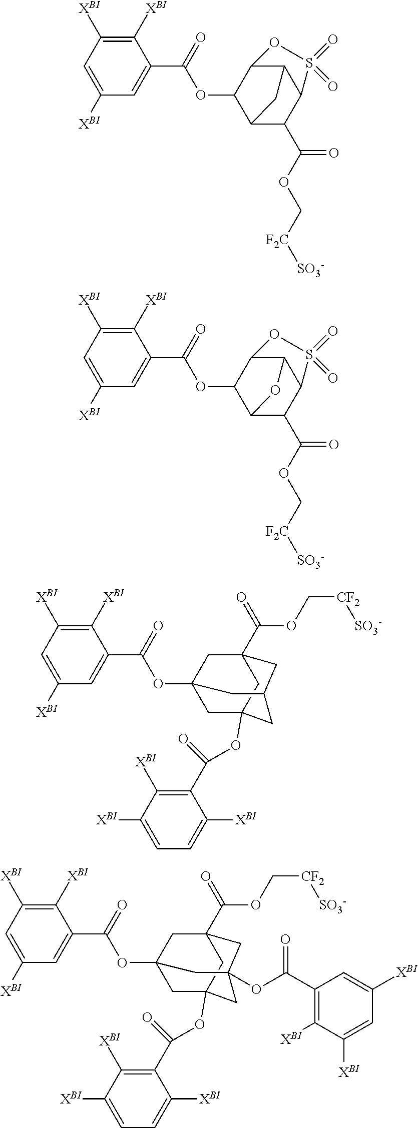

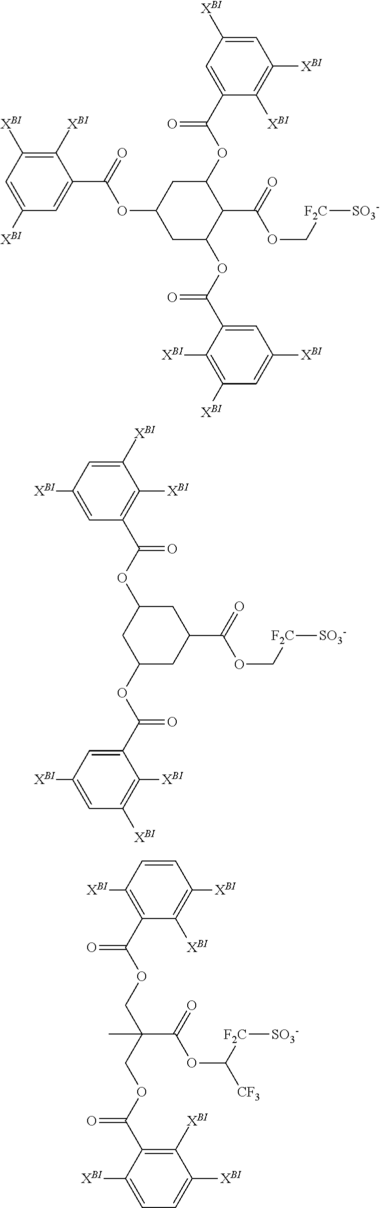

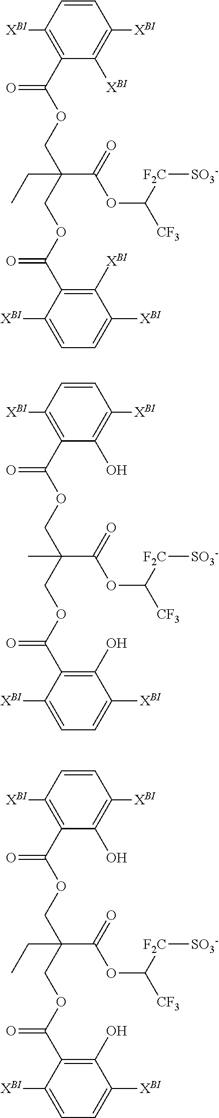

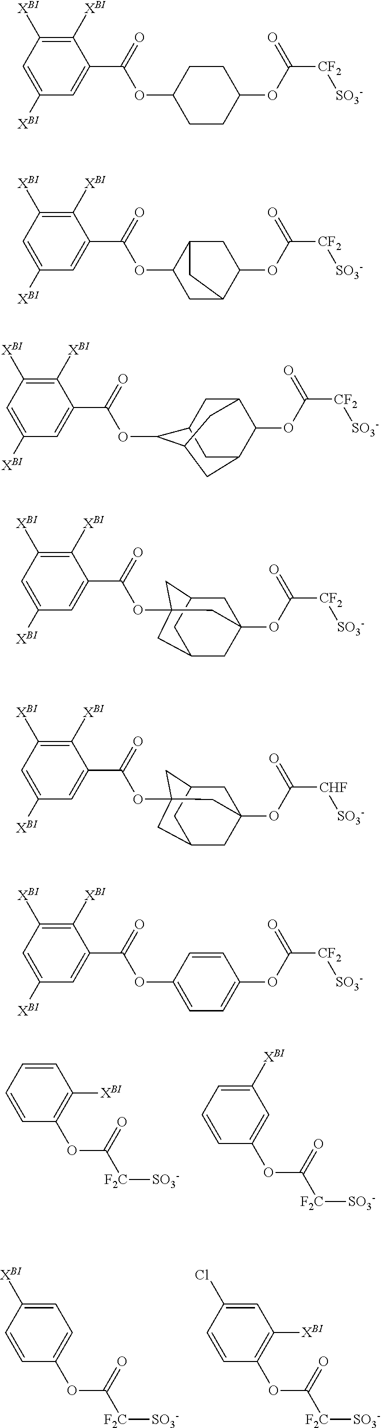

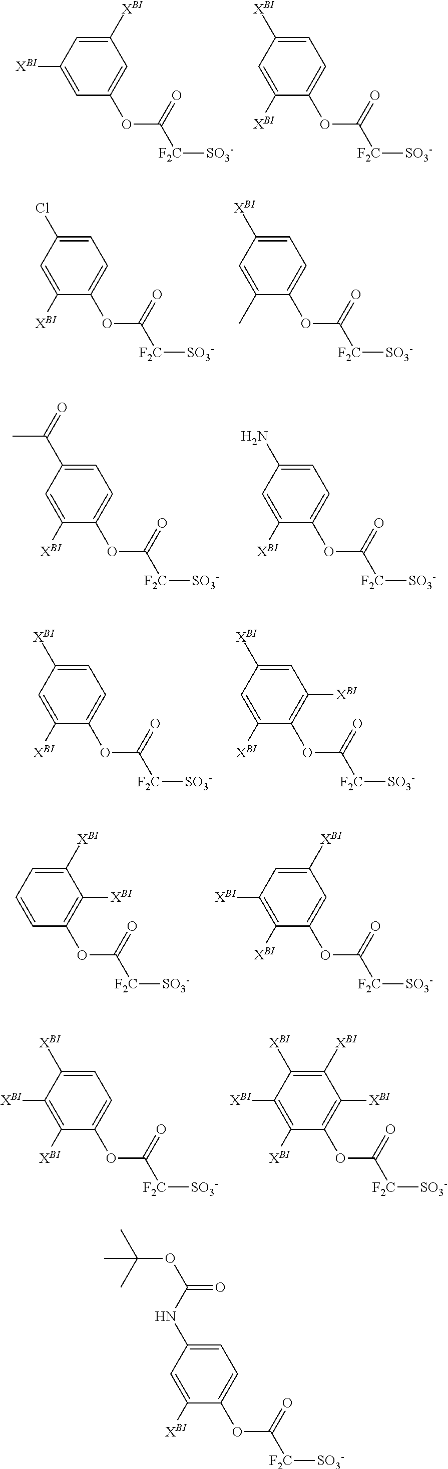

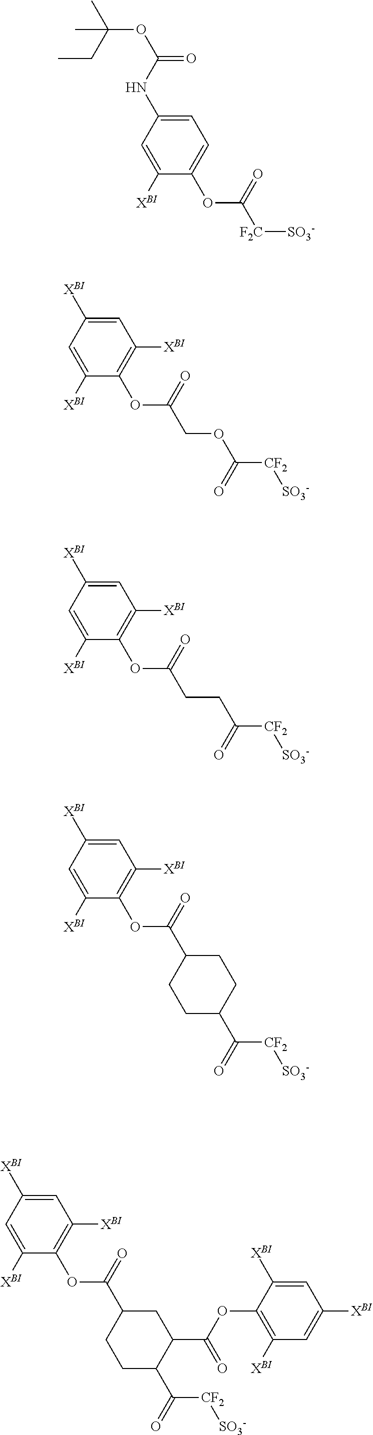

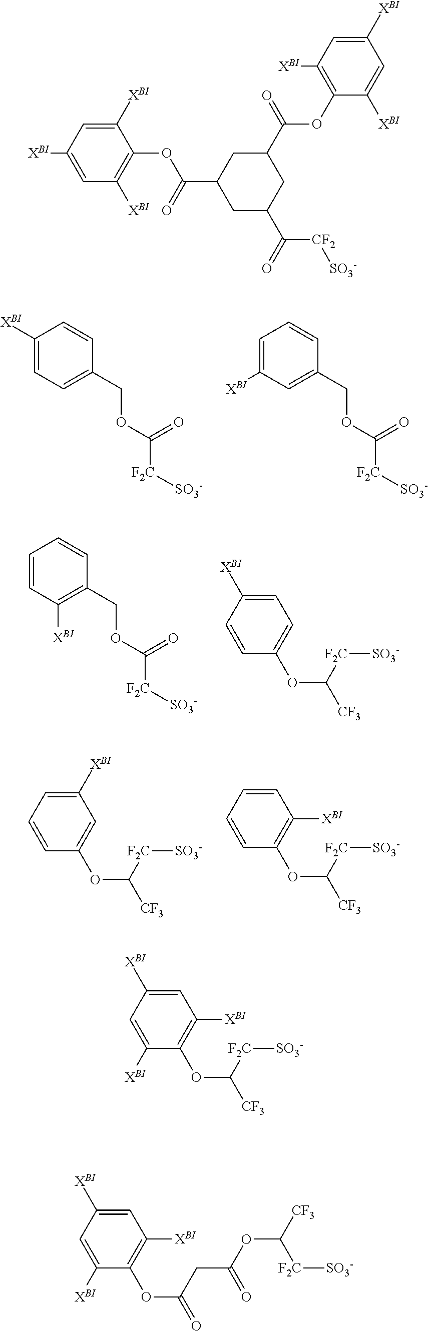

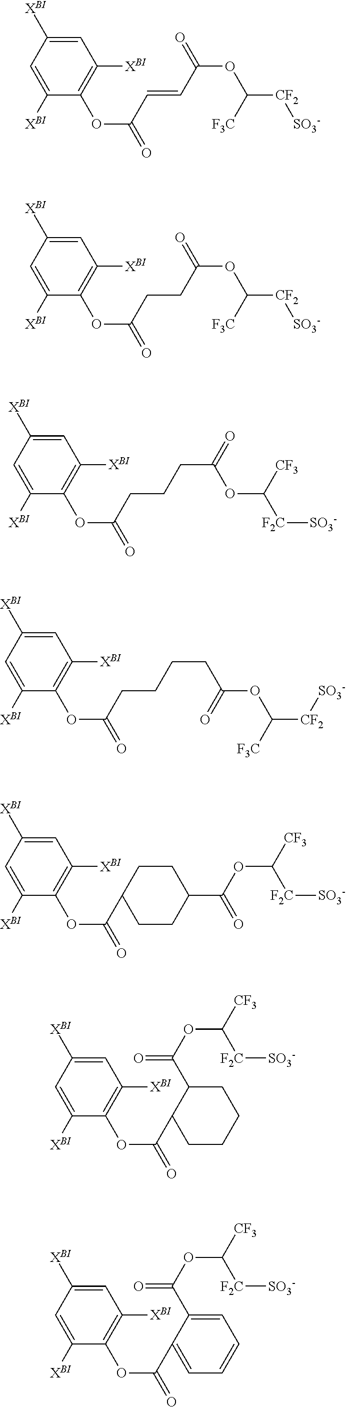

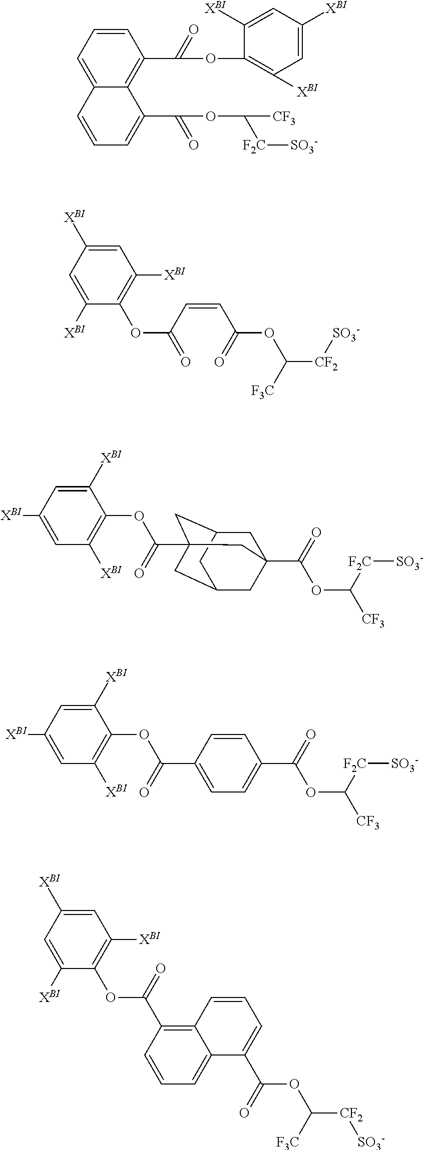

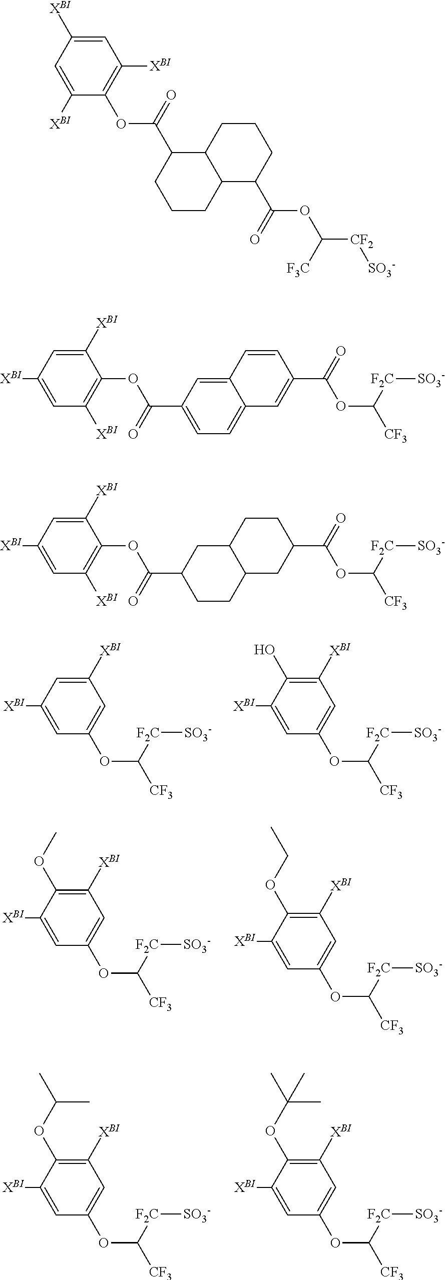

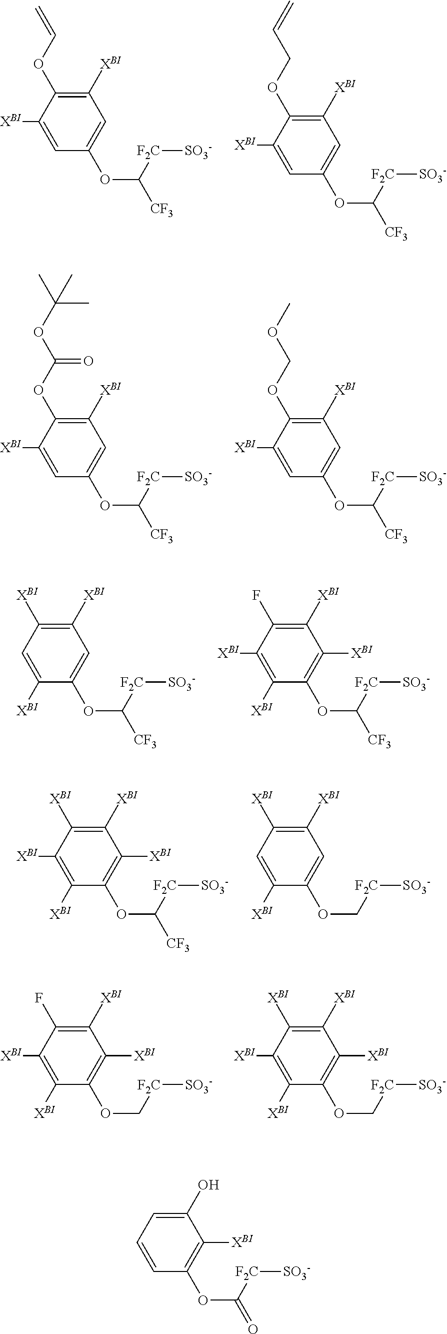

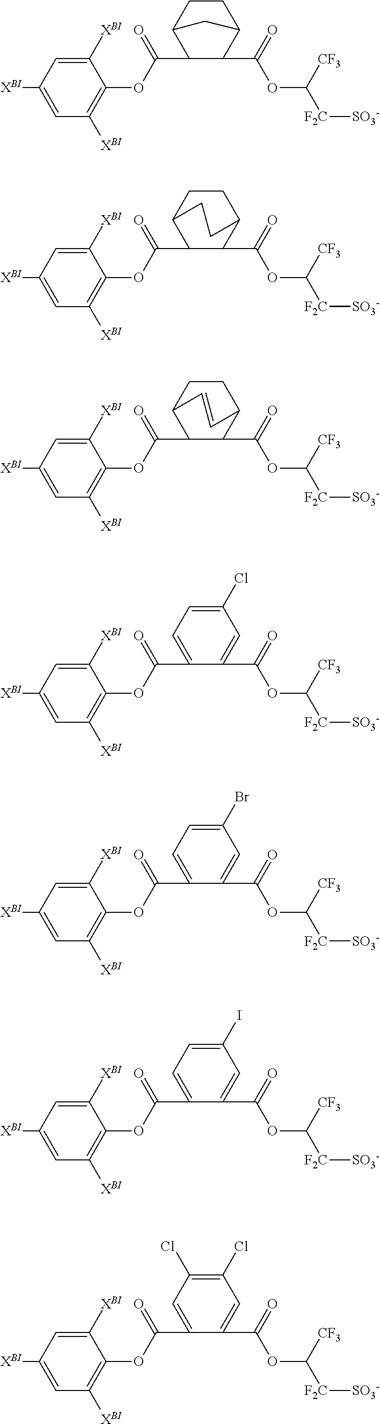

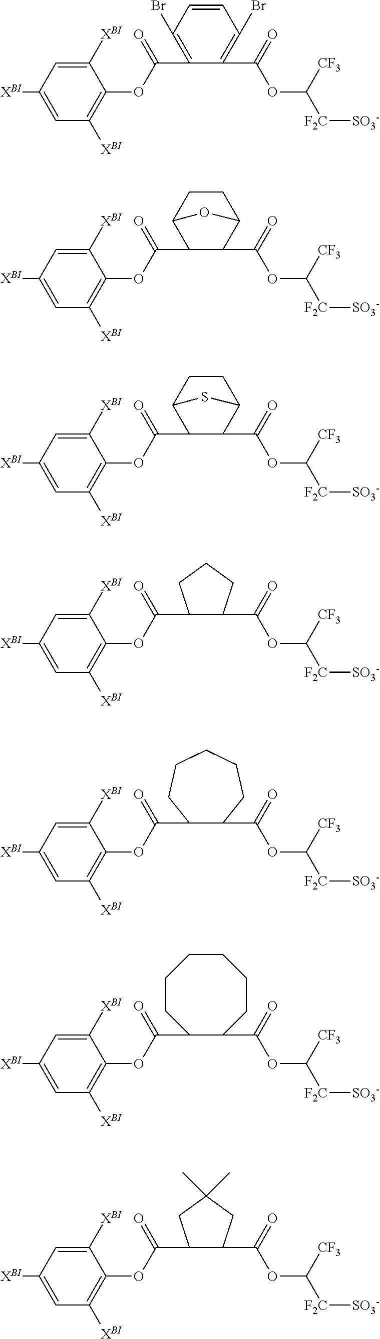

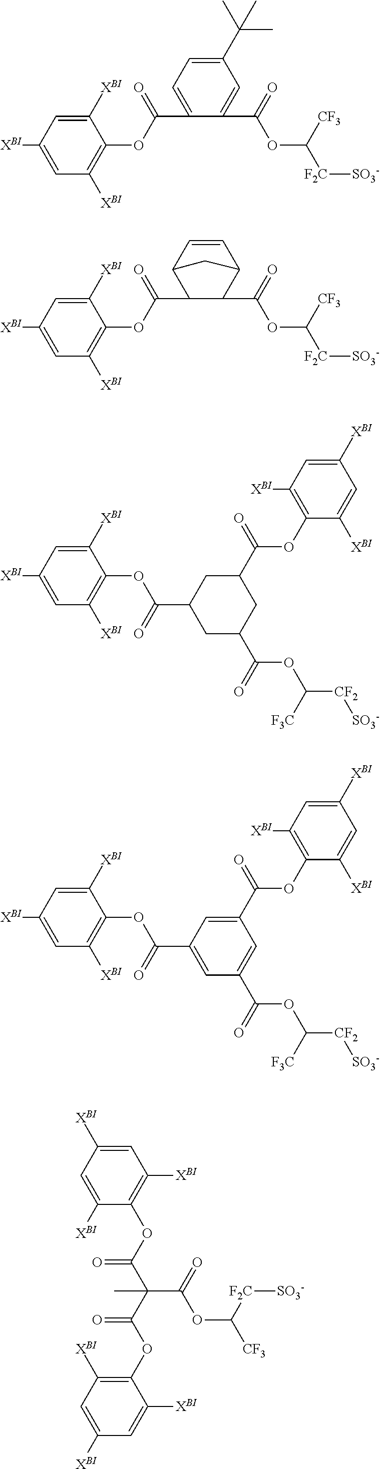

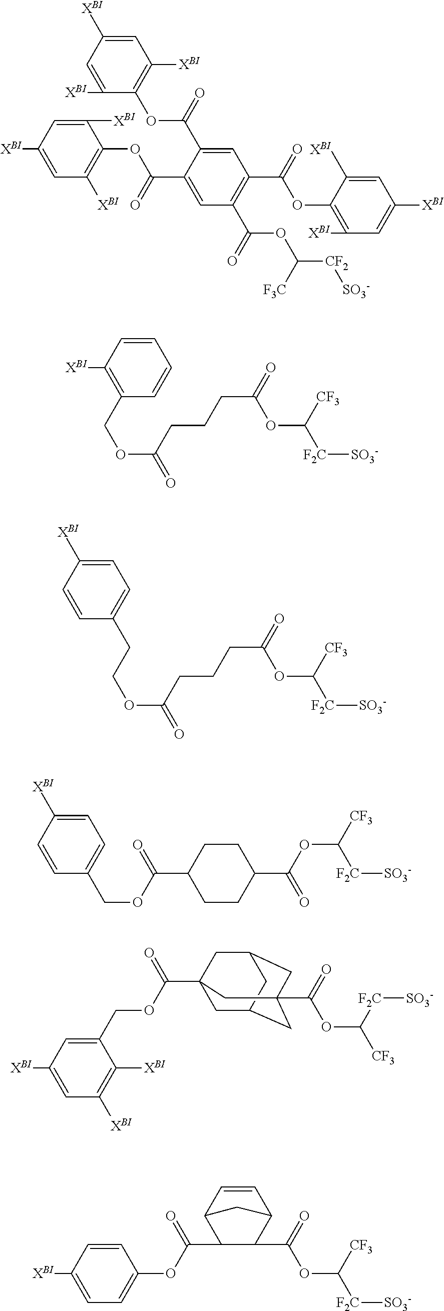

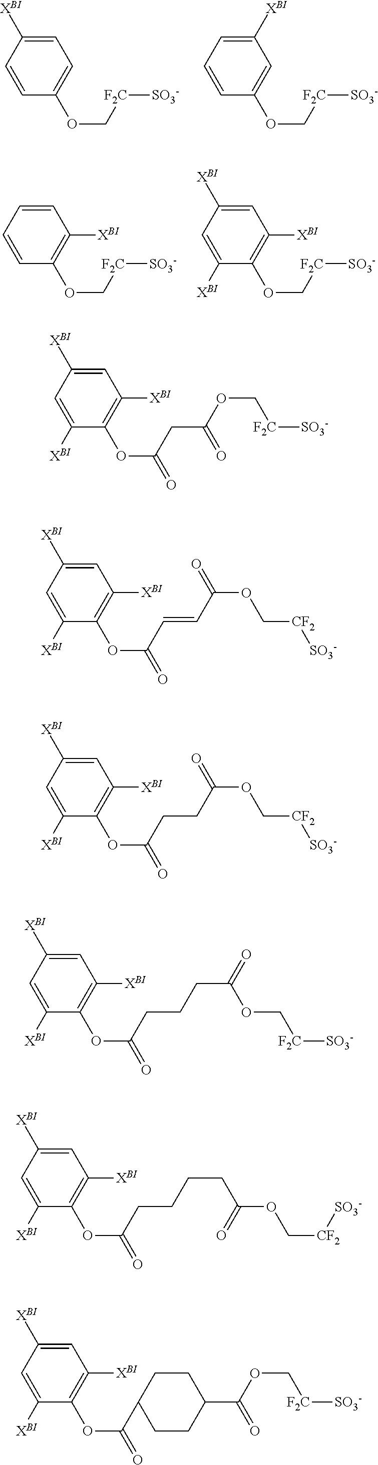

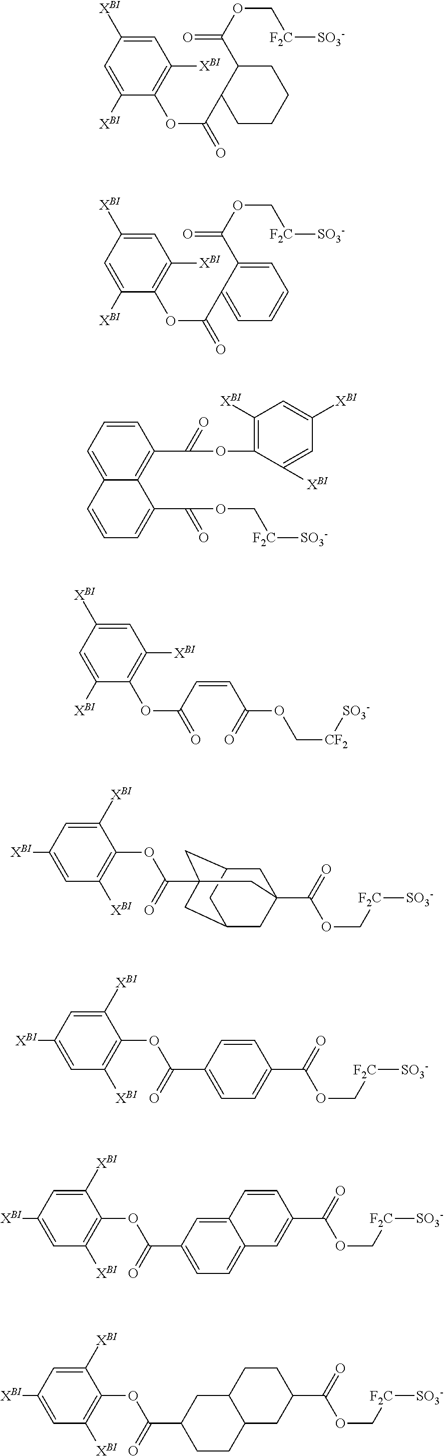

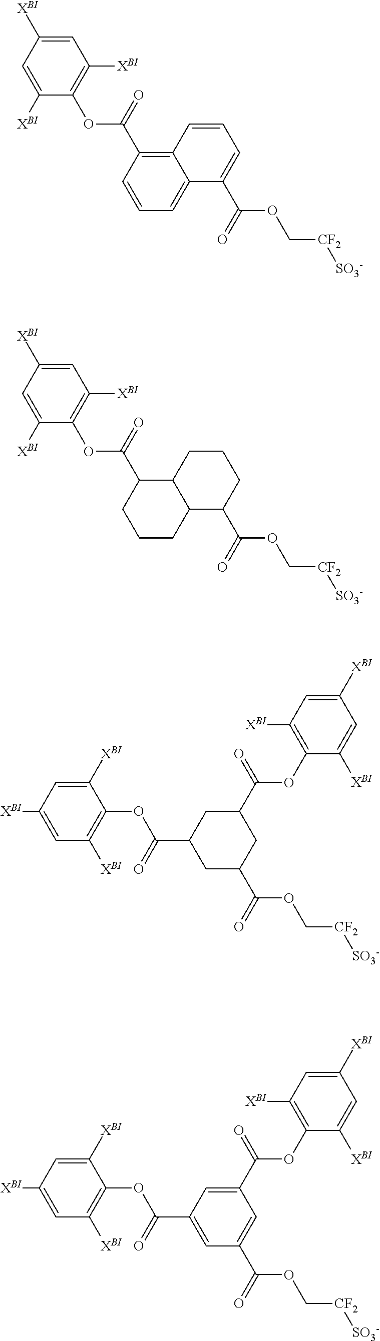

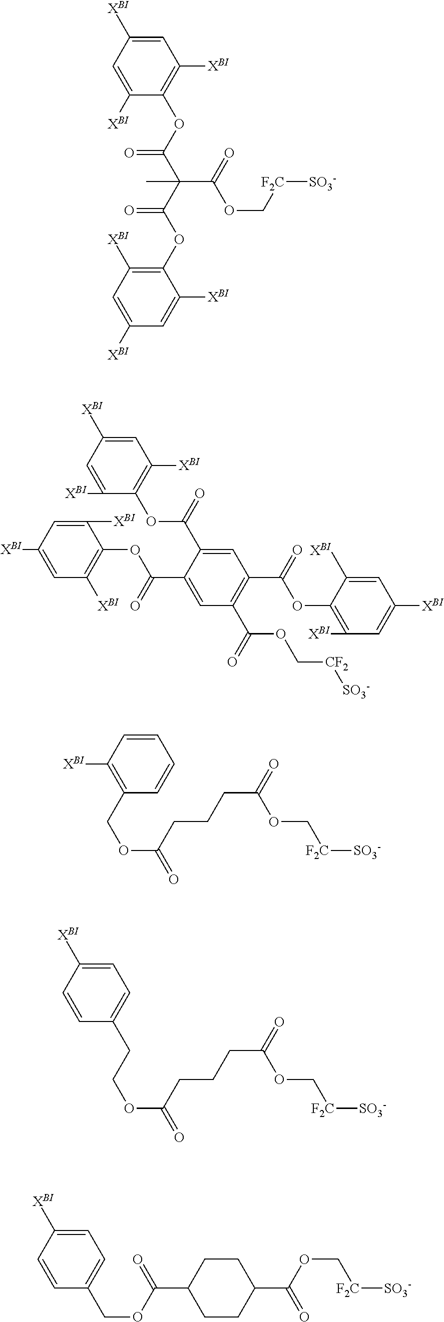

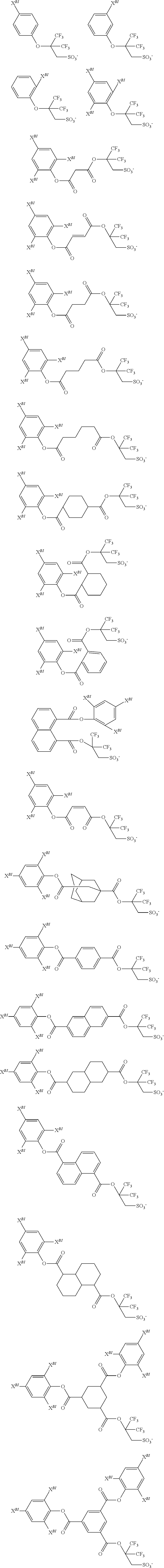

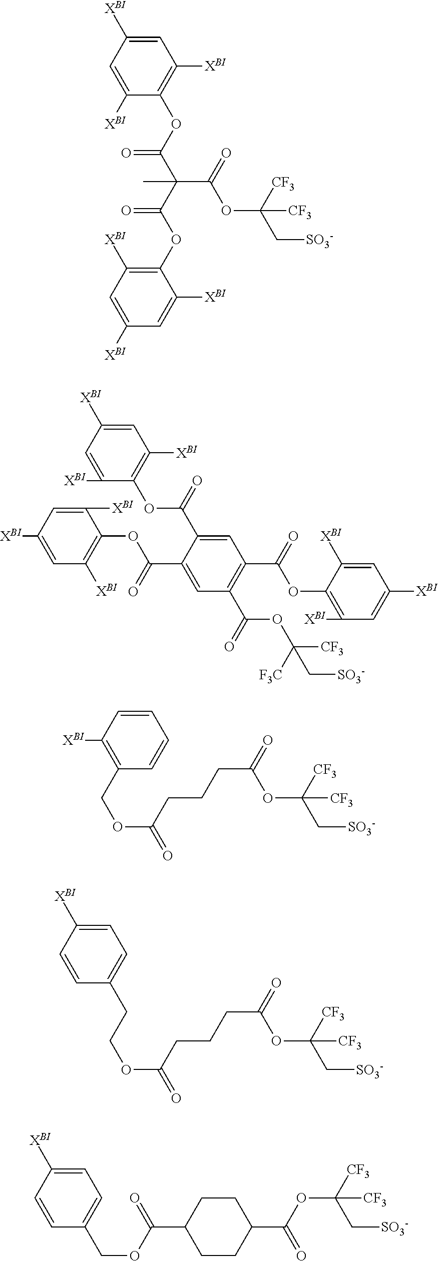

Other useful example of the non-nucleophilic counter ion include fluorobenzenesulfonic acid anions having an iodized aromatic ring bonded thereto as described in JP 6648726, anions having an acid-catalyzed decomposition mechanism as described in WO 2021/200056 and JP-A 2021-070692, anions having a cyclic ether group as described in JP-A 2018-180525 and JP-A 2021-035935, and anions as described in JP-A 2018-092159.

Further useful examples of the non-nucleophilic counter ion include bulky fluorine-free benzenesulfonic acid anions as described in JP-A 2006-276759, JP-A 2015-117200, JP-A 2016-065016, and JP-A 2019-202974; fluorine-free benzenesulfonic acid or alkylsulfonic acid anions having an iodized aromatic group bonded thereto as described in JP 6645464.

Also useful are bissulfonic acid anions as described in JP-A 2015-206932, sulfonamide or sulfonimide anions having sulfonic acid side and different side as described in WO 2020/158366, and anions having a sulfonic acid side and a carboxylic acid side as described in JP-A 2015-024989.

In formulae (c2) and (c3), d1 and d2 are each independently 0, 1, 2 or 3, preferably 1.

In formula (c4), e1 is 0 or 1, e2 is 0, 1, 2, 3 or 4, and e3 is 0, 1, 2, 3 or 4, meeting 0≤e2+e3≤when e1=0, and 0≤e2+e3≤6 when e1=1.

In formulae (c2), (c3) and (c4), L¹ is a single bond, ether bond, ester bond, carbonyl, sulfonate ester bond, sulfonamide bond, carbonate bond or carbamate bond. From the aspect of synthesis, an ether bond, ester bond or carbonyl is preferred, with the ester bond or carbonyl being more preferred.

In formula (c2), Rf¹ and R² are each independently fluorine or a C₁-C₆ fluorinated saturated hydrocarbyl group. It is preferred that both Rf¹ and Rf² be fluorine because the generated acid has a higher acid strength. Rf³ and Rf⁴ are each independently hydrogen, fluorine or a C₁-C₆ fluorinated saturated hydrocarbyl group. It is preferred for solvent solubility that at least one of Rf³ and Rf⁴ be trifluoromethyl.

In formula (c3), Rf⁵ and Rf⁶ are each independently hydrogen, fluorine or a C₁-C₆ fluorinated saturated hydrocarbyl group. It is excluded that all Rf⁵ and Rf⁶ are hydrogen at the same time. It is preferred for solvent solubility that at least one of Rf⁵ and Rf⁶ be trifluoromethyl.

In formula (c4), Rf⁷ is fluorine, a C₁-C₆ fluorinated alkyl group, C₁-C₆ fluorinated alkoxy group, or C₁-C₆ fluorinated alkylthio group. Rf⁷ is preferably fluorine, trifluoromethyl, difluoromethyl, trifluoromethoxy, difluoromethoxy, trifluoromethylthio or difluoromethylthio, more preferably fluorine, trifluoromethyl or trifluoromethoxy. When e2 is 2, 3 or 4, a plurality of Rf⁷ may be identical or different.

In formula (c4), R³³ is halogen exclusive of fluorine, or a C₁-C₂₀ hydrocarbyl group which may contain a heteroatom. The hydrocarbyl group may be saturated or unsaturated and straight, branched or cyclic. Examples thereof are as exemplified above for the hydrocarbyl group R¹ in formula (A), but not limited thereto. When e3 is 2, 3 or 4, a plurality of R³³ may be identical or different.

When e3 is 2, 3 or 4, a plurality of R³³ may bond together to form a ring with the carbon atoms to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

Examples of the anion in repeat unit (c2) are shown below, but not limited thereto. R^{A} is as defined above.

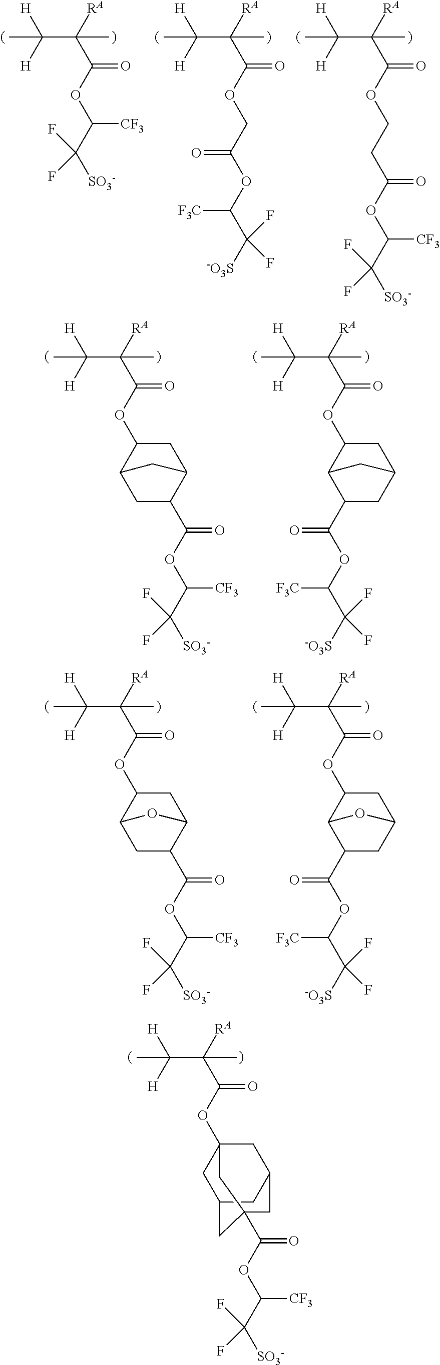

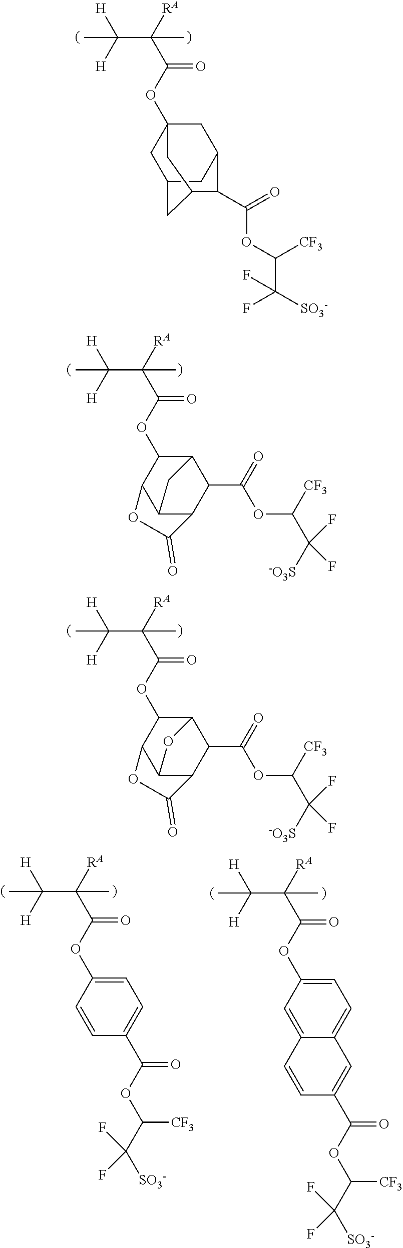

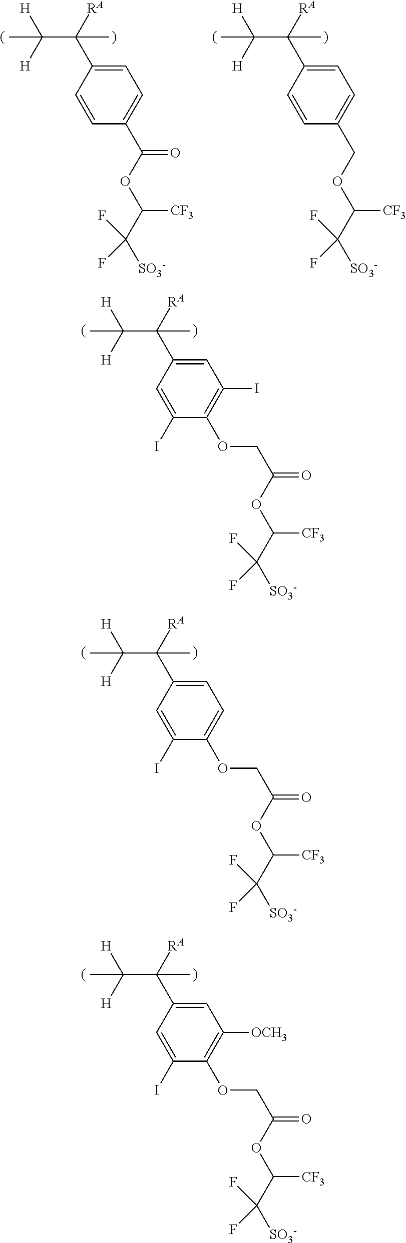

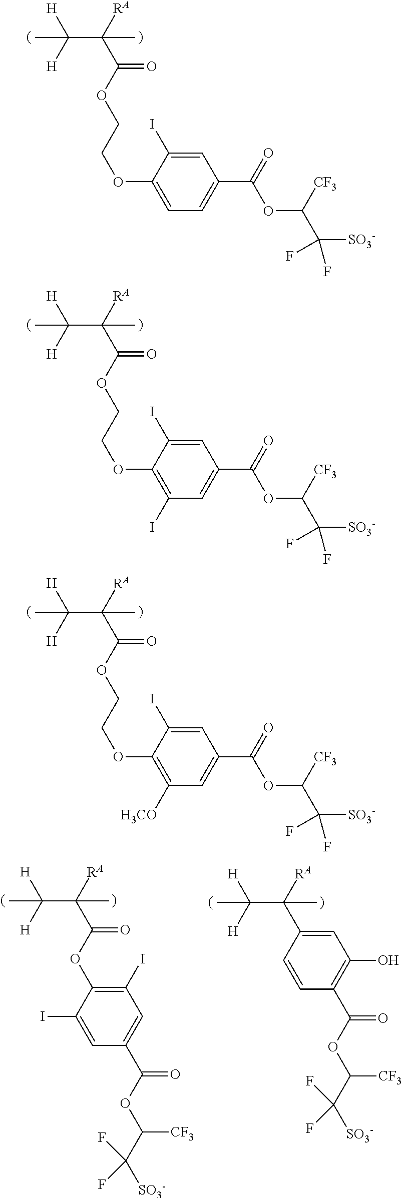

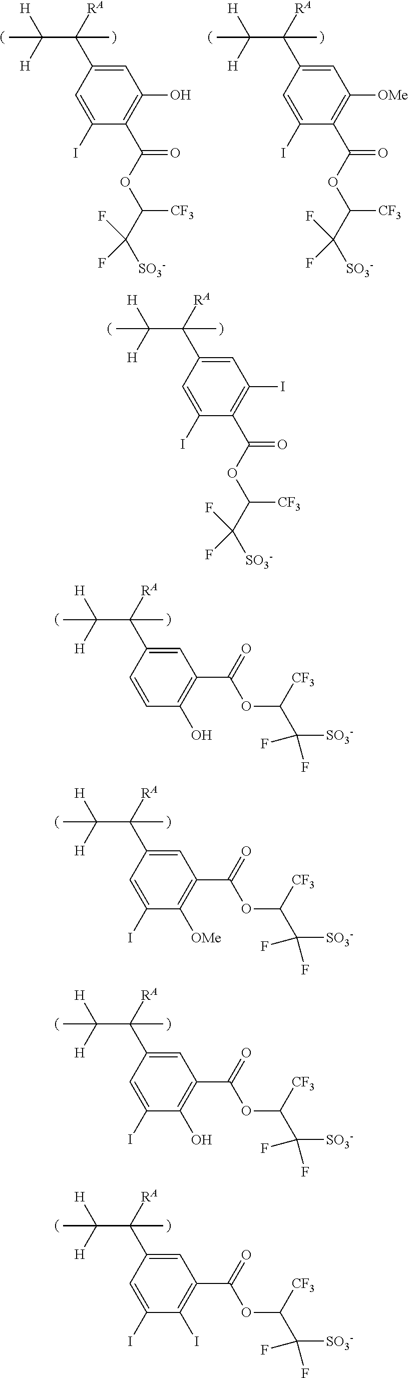

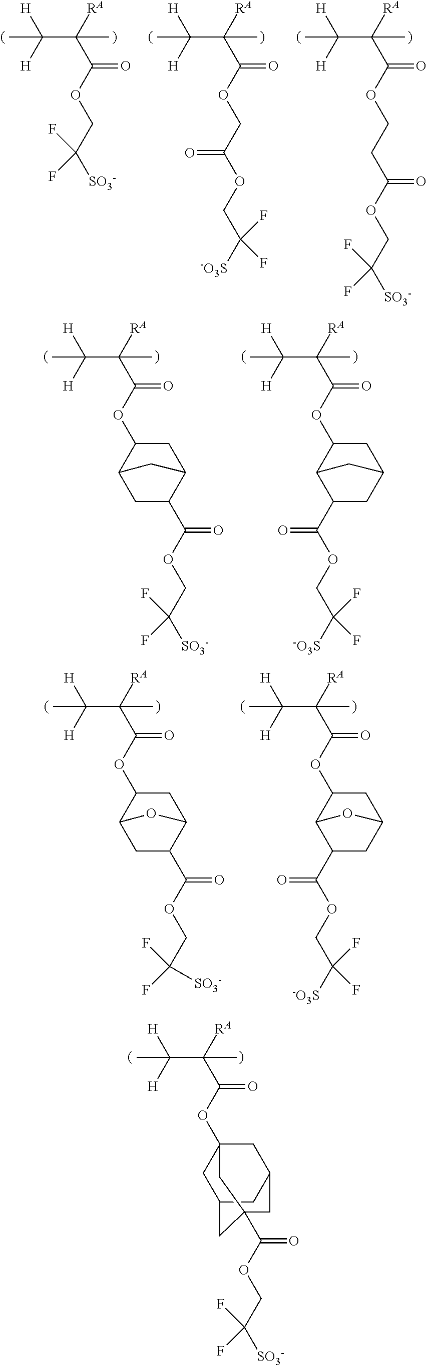

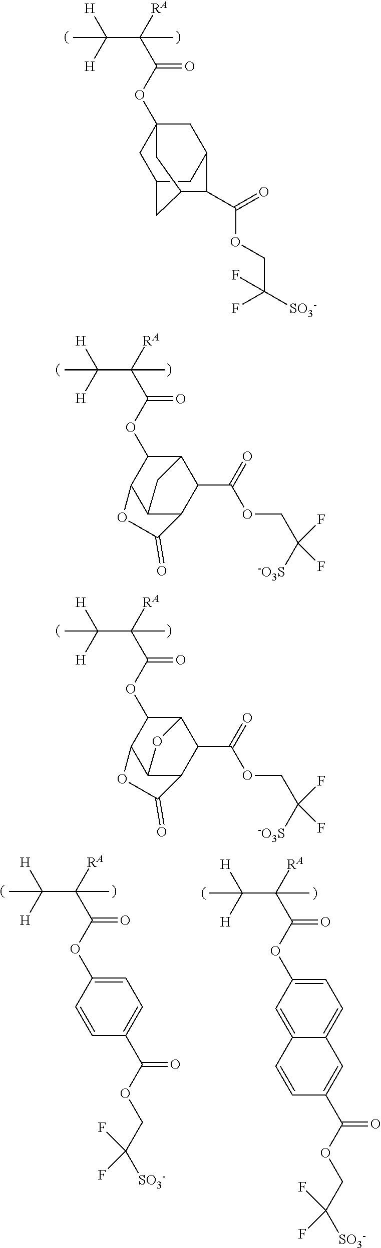

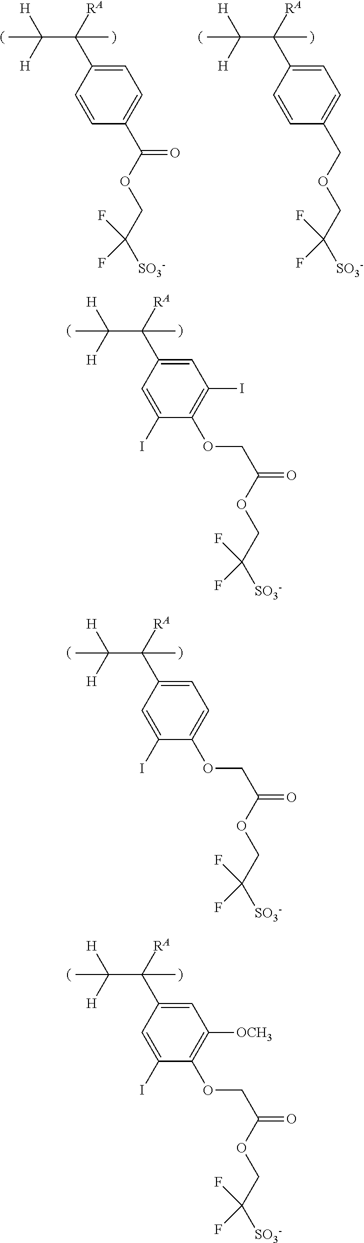

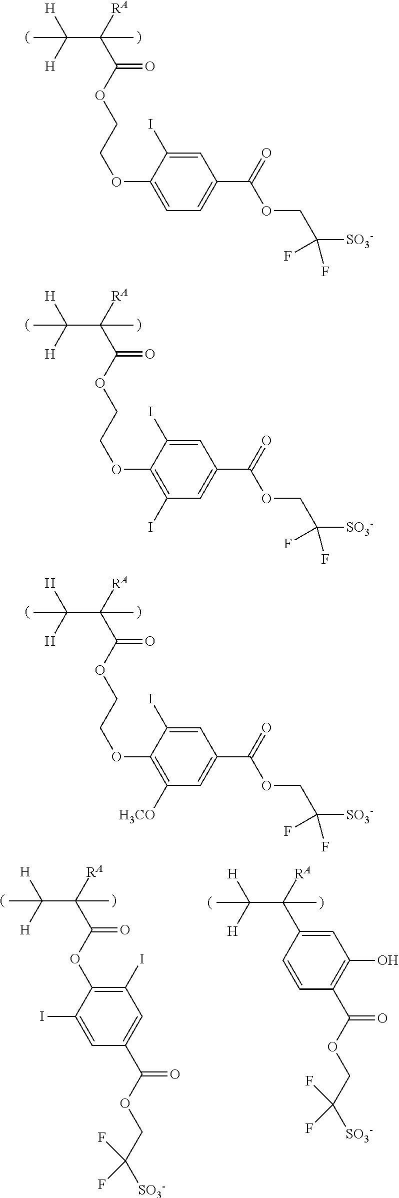

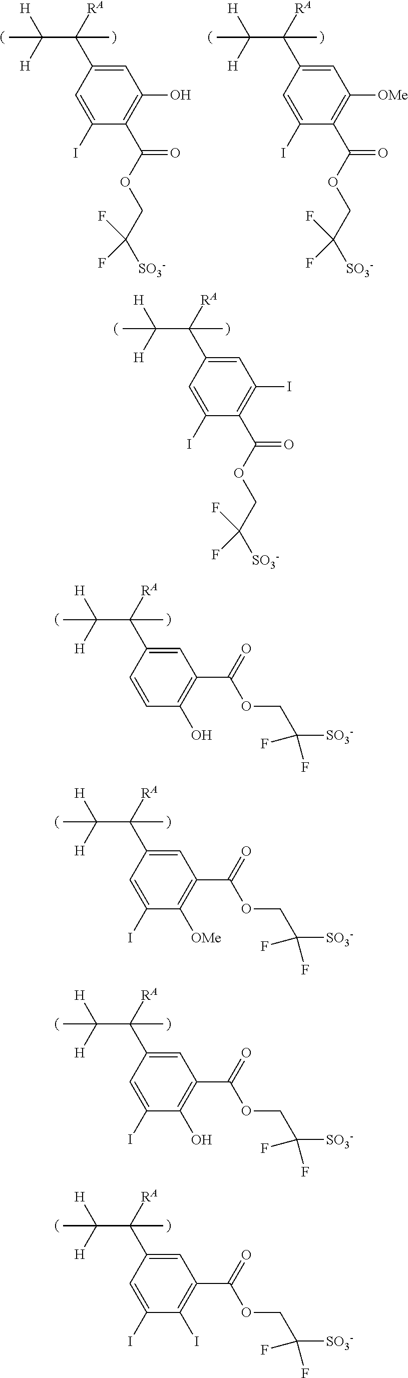

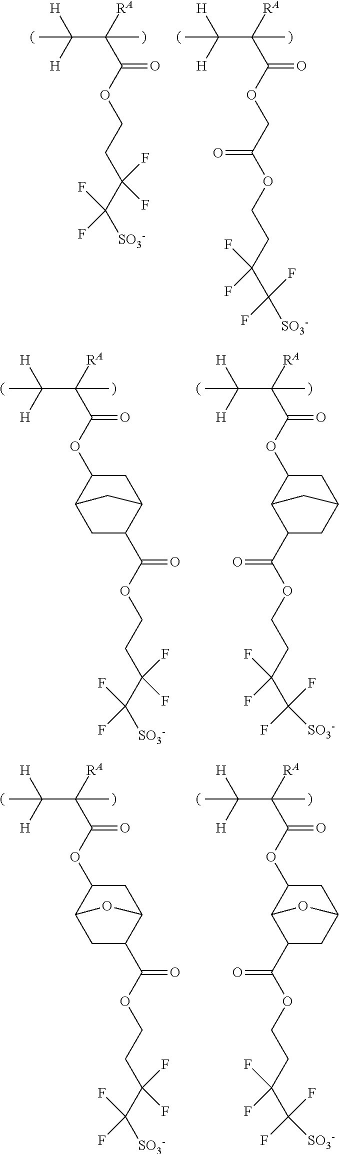

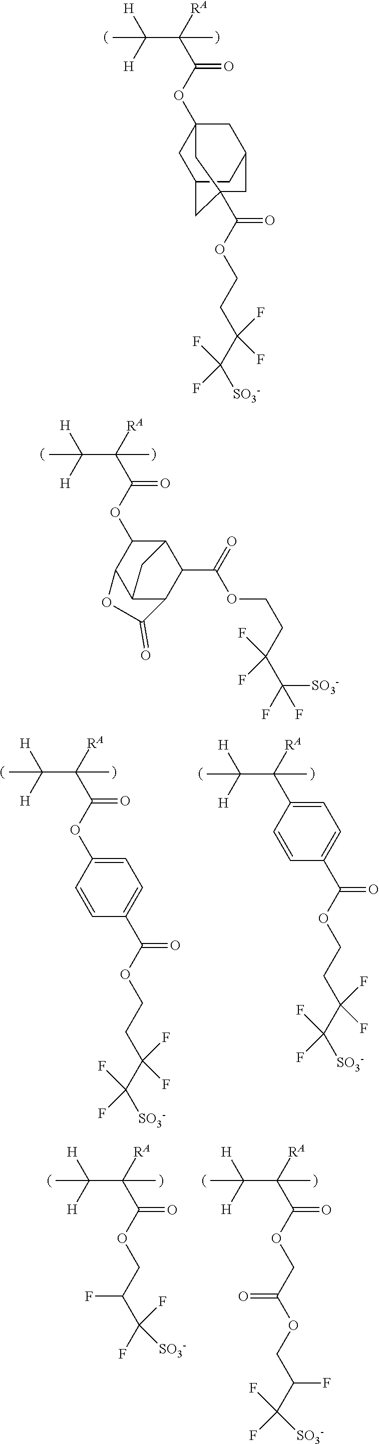

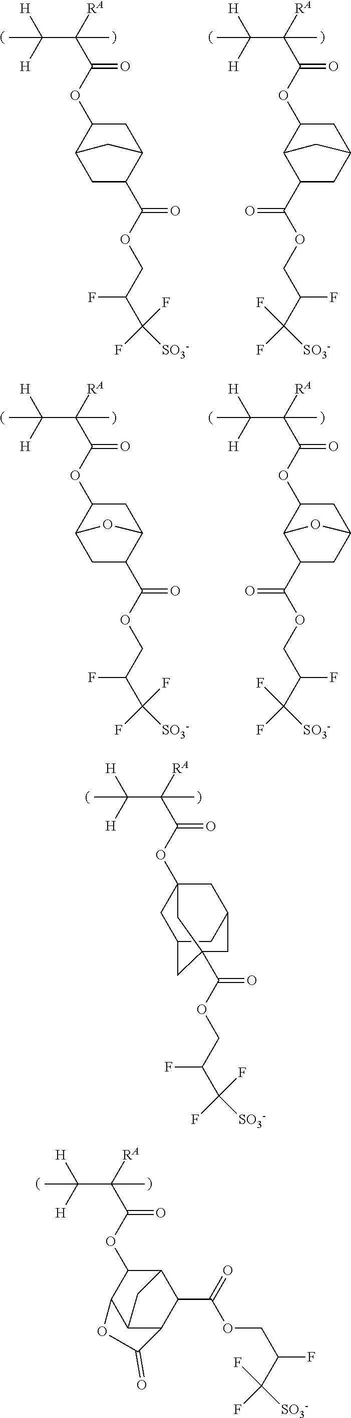

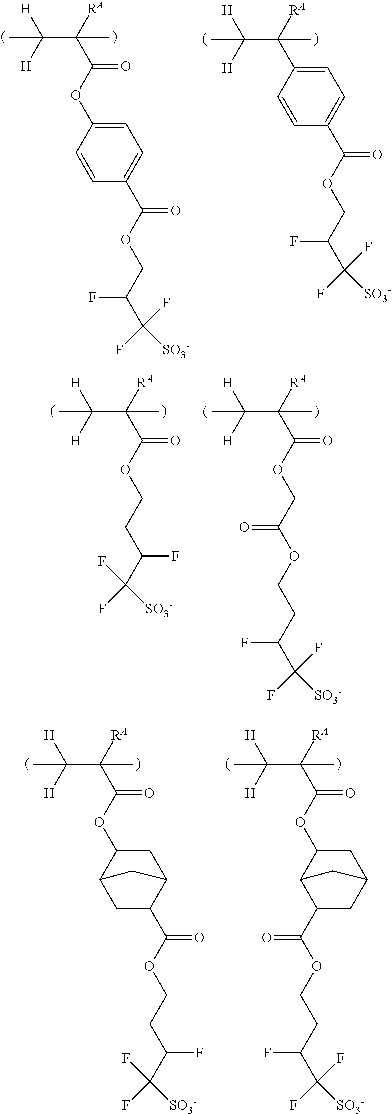

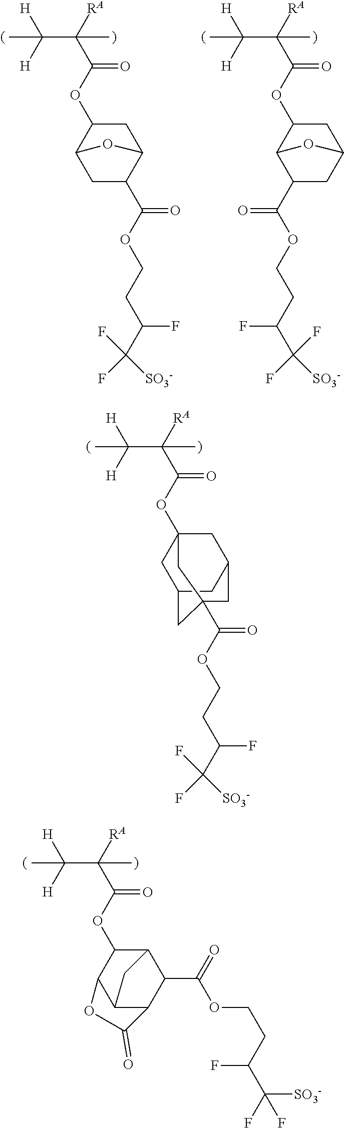

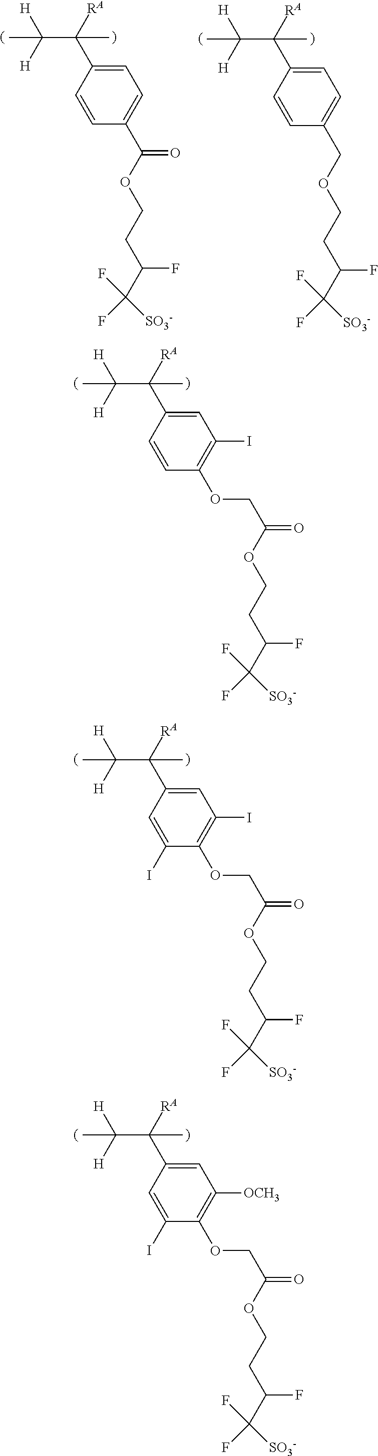

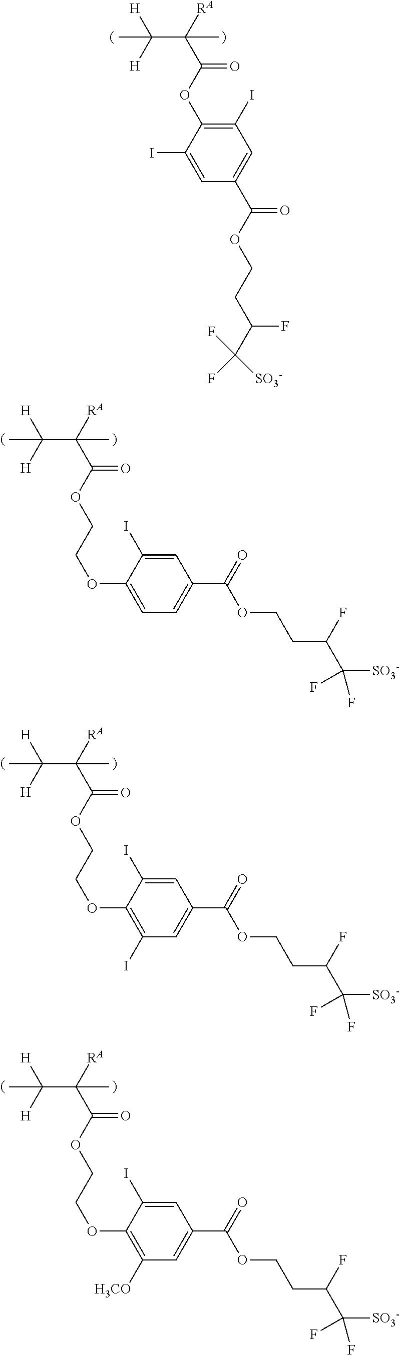

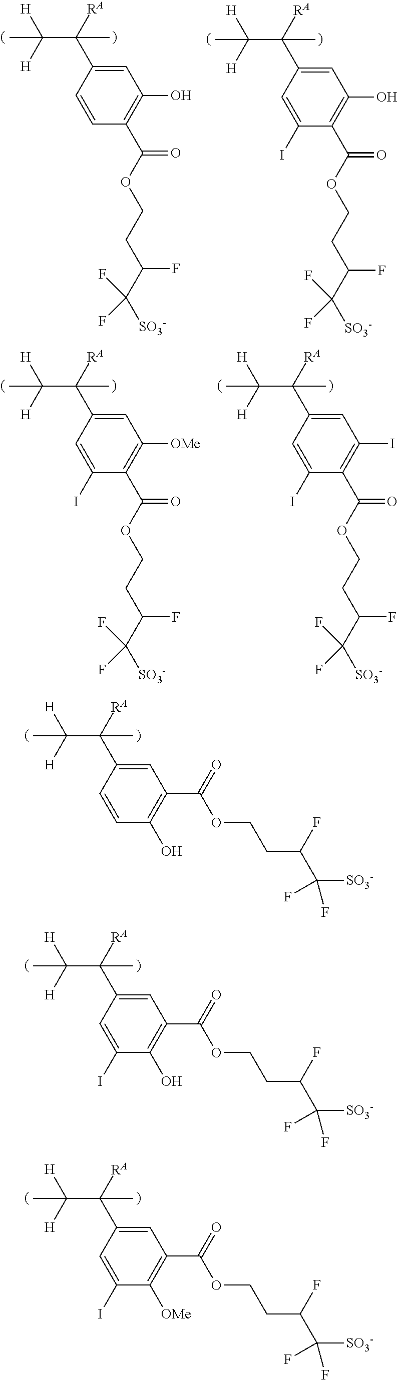

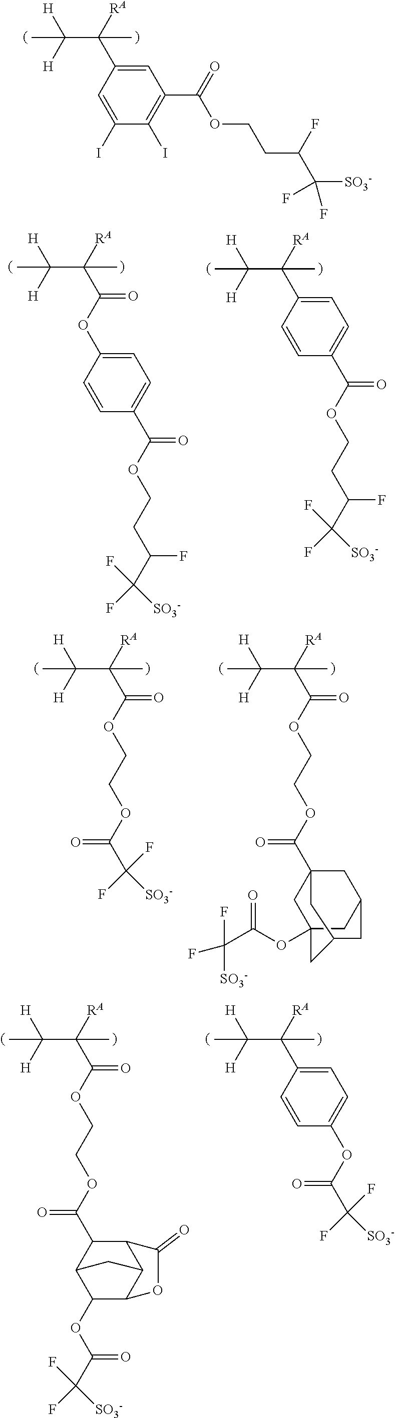

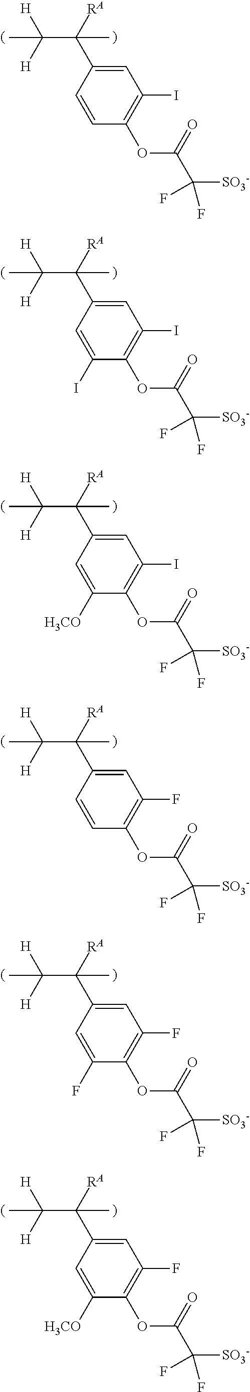

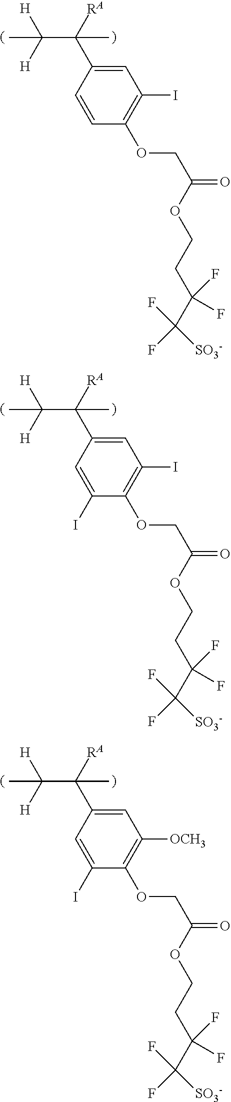

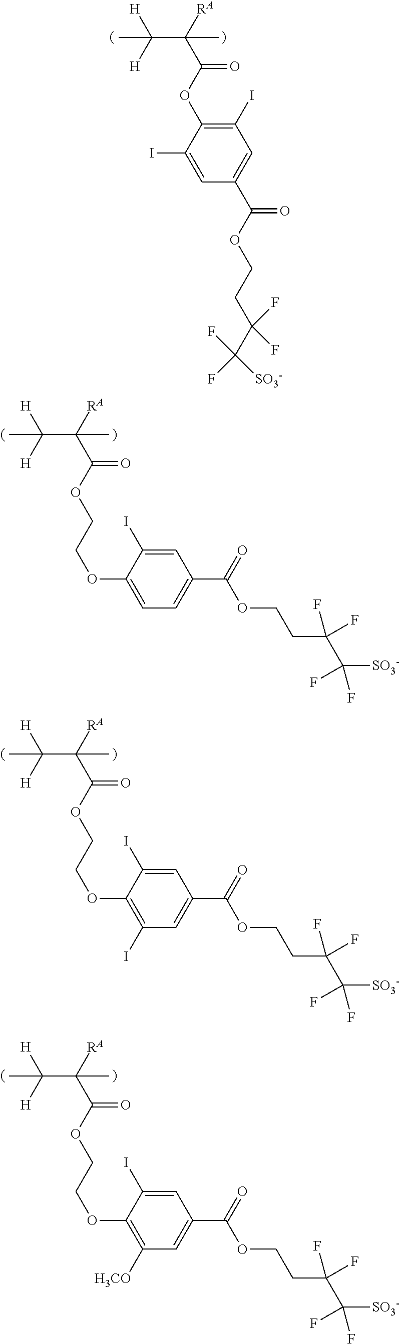

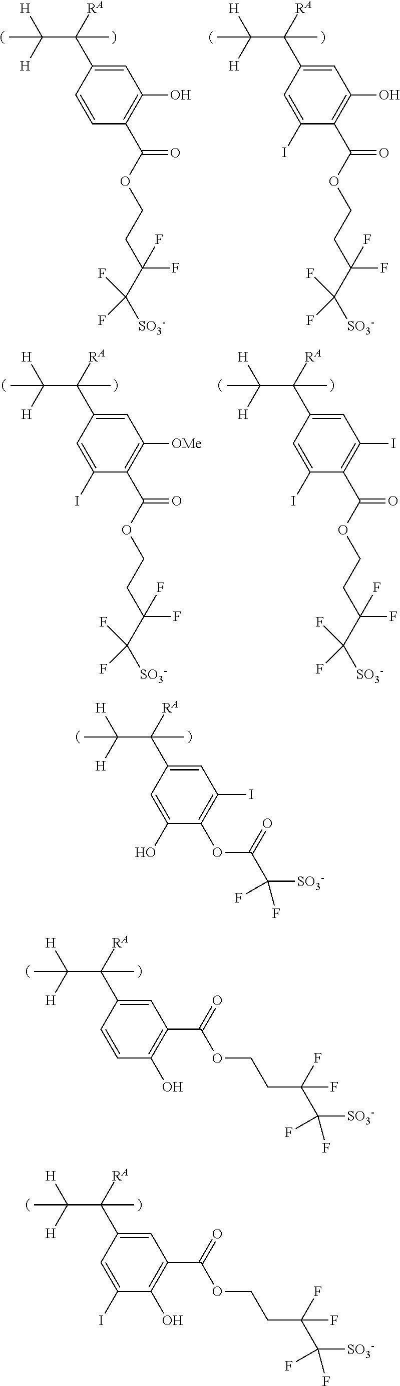

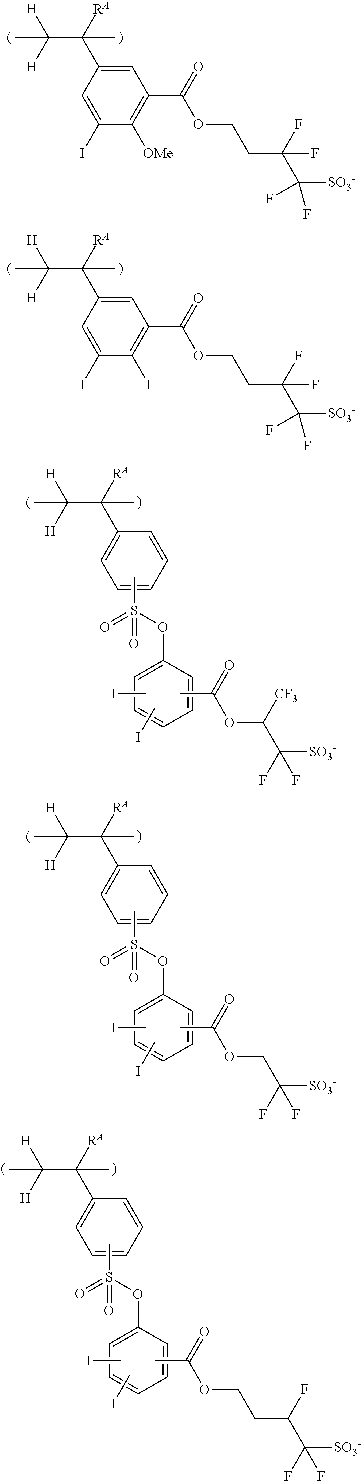

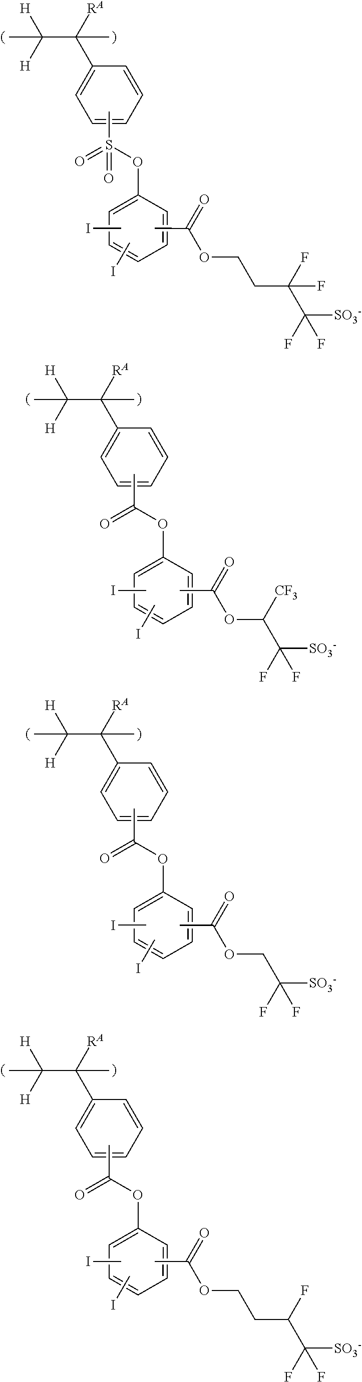

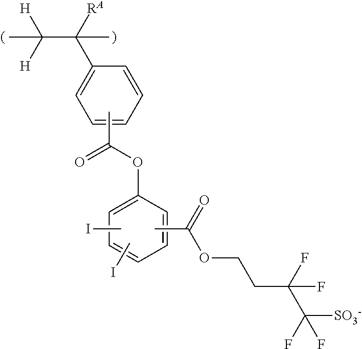

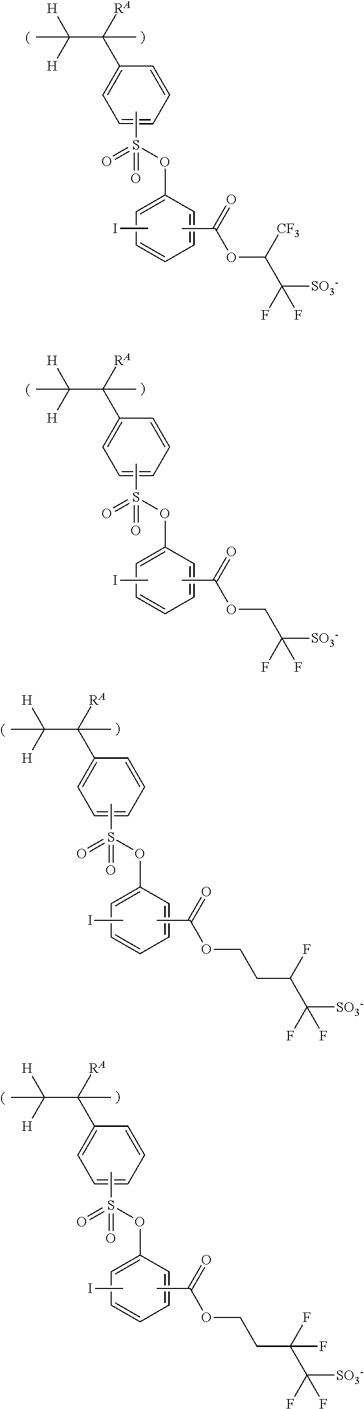

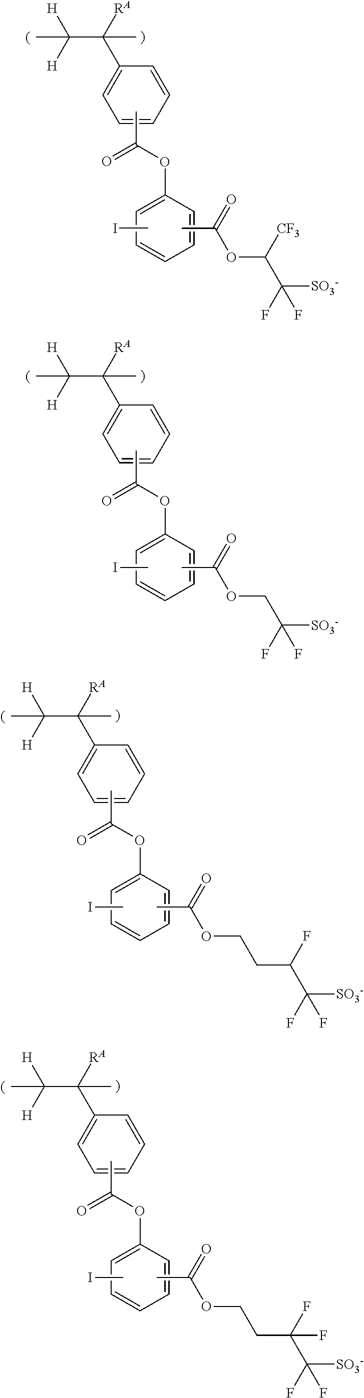

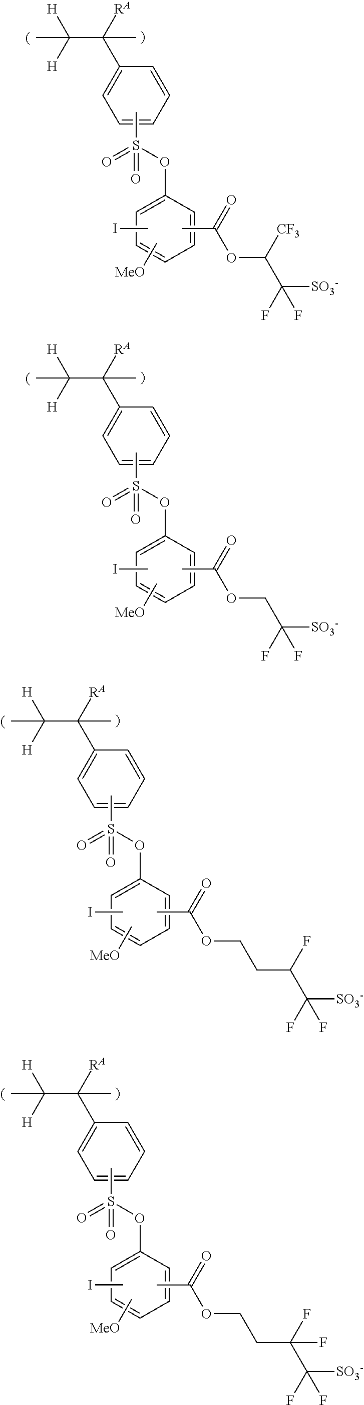

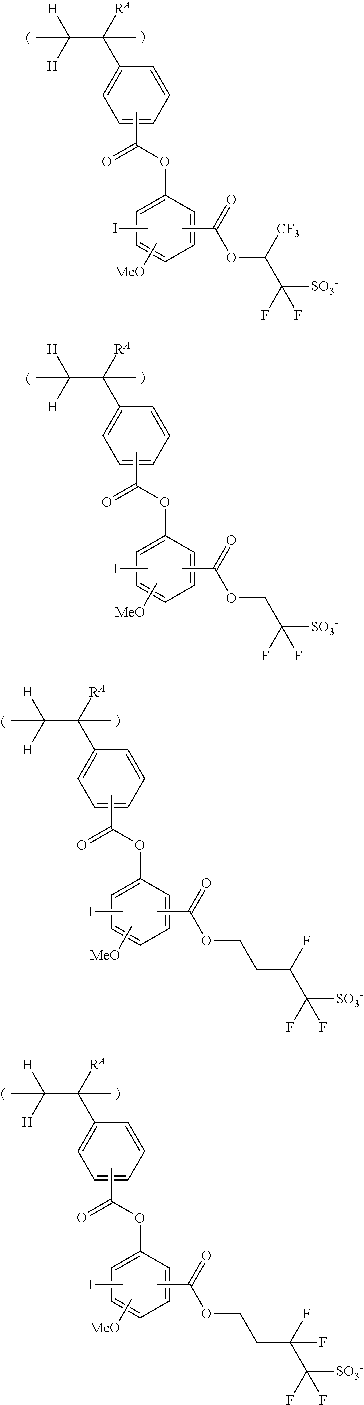

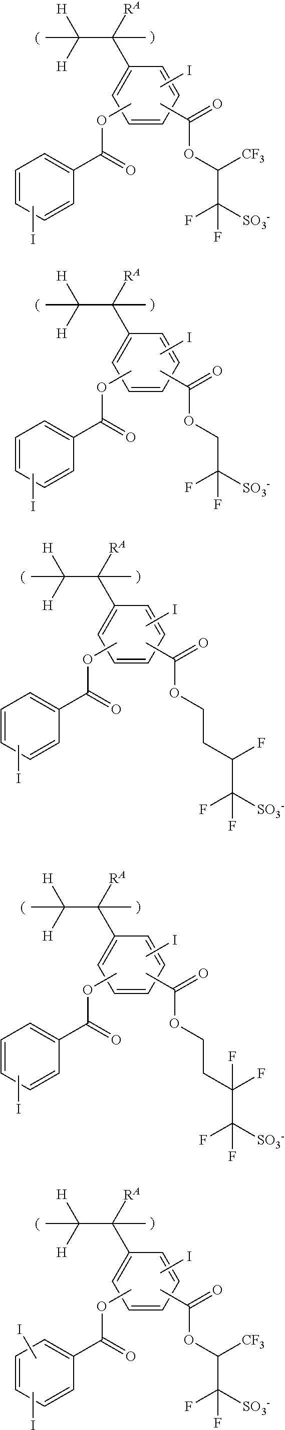

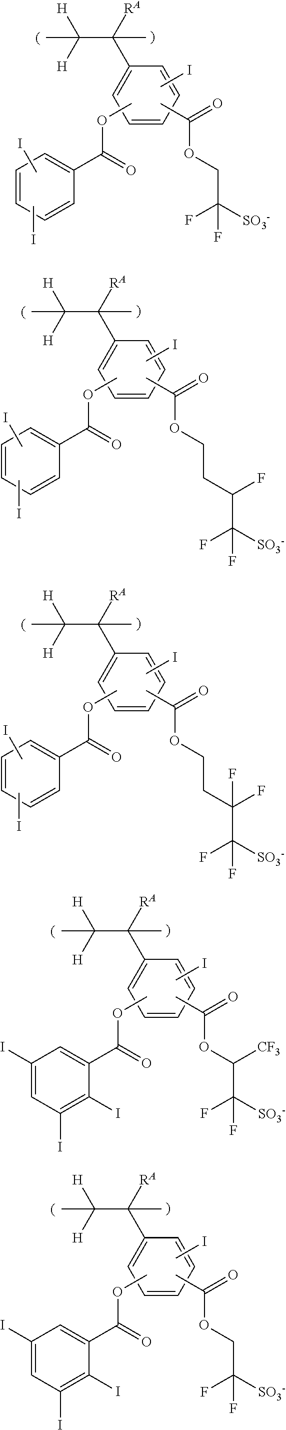

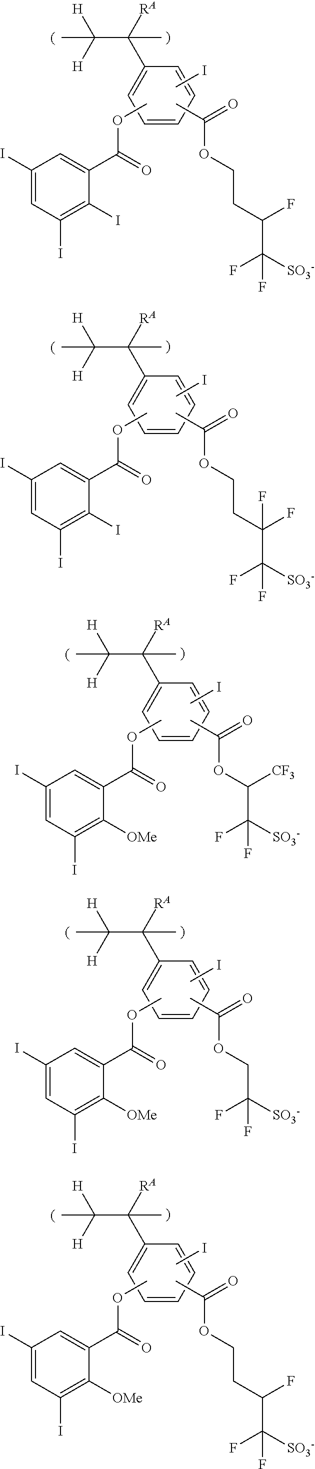

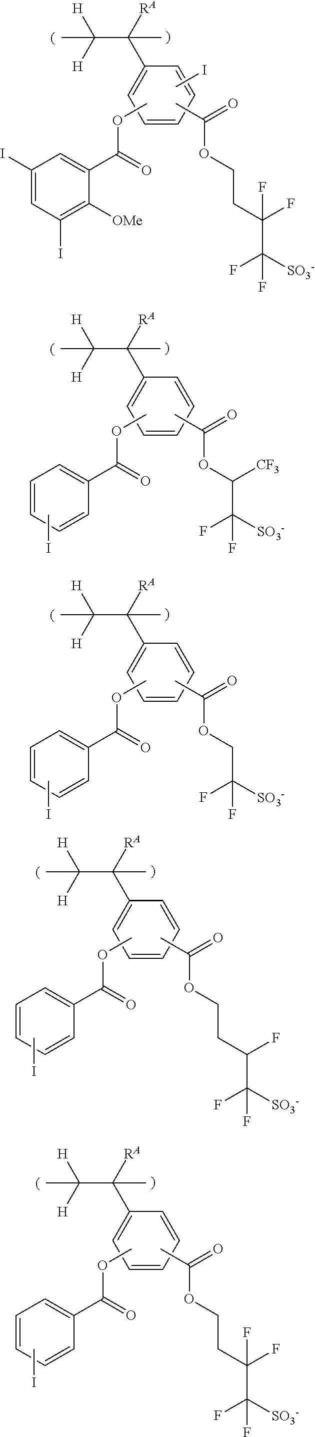

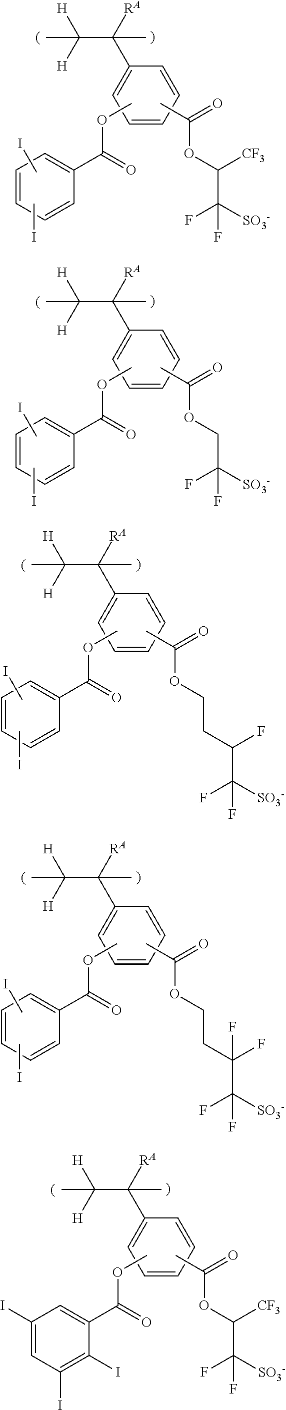

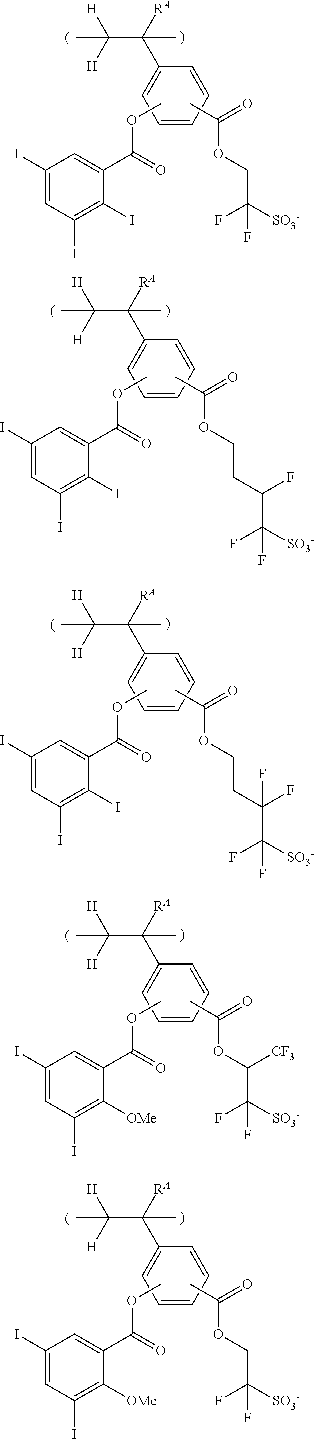

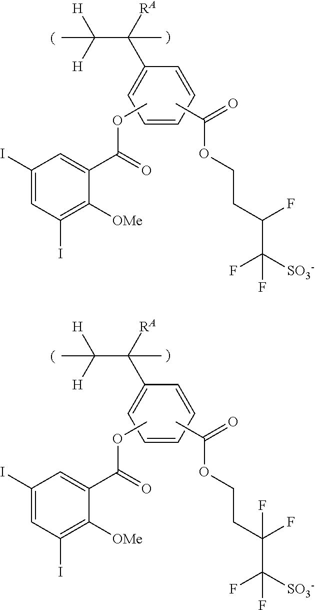

Examples of the anion in repeat unit (6) are shown below, but not limited thereto. R^{A} is as defined above.

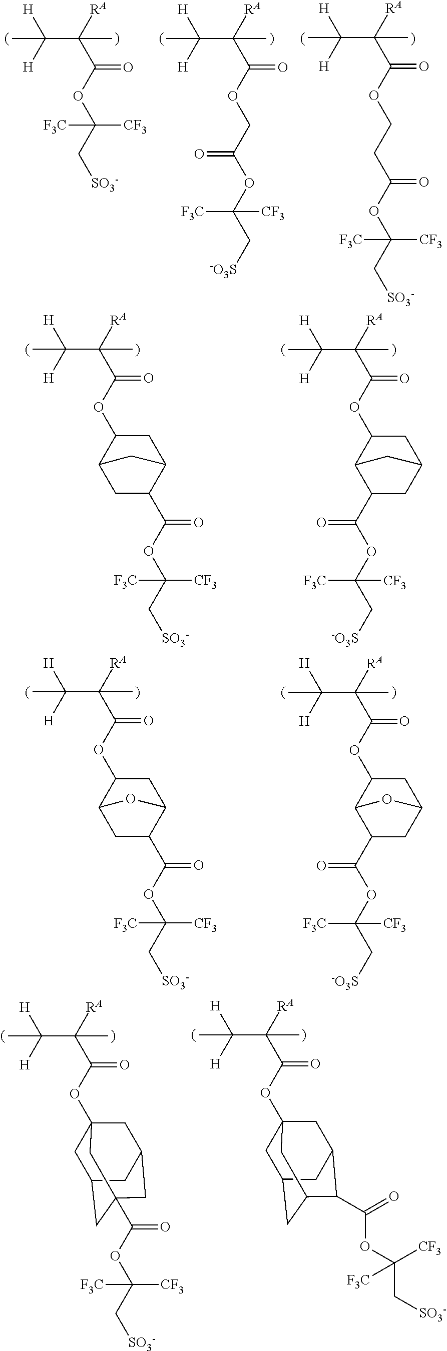

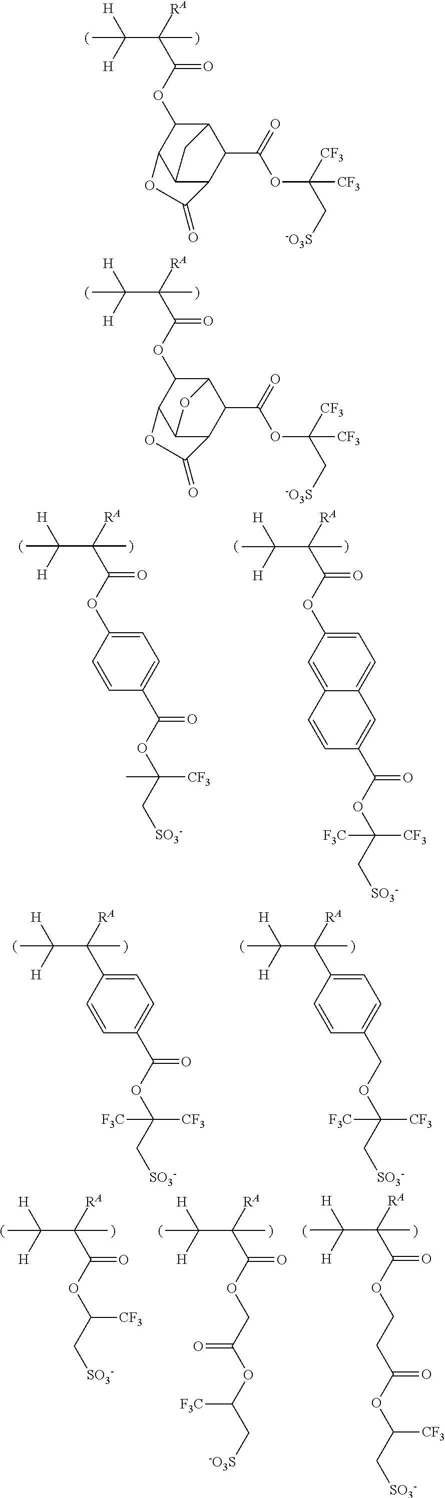

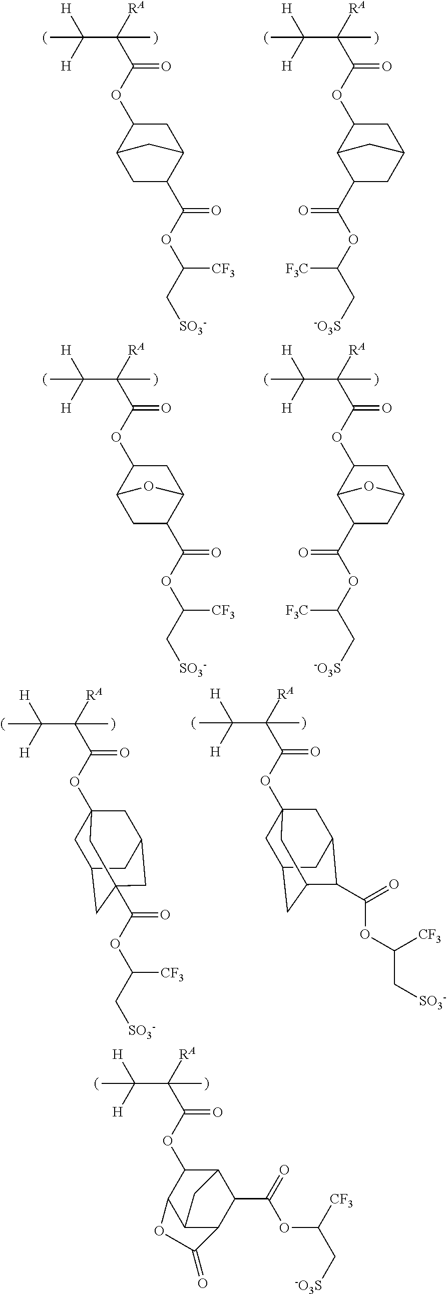

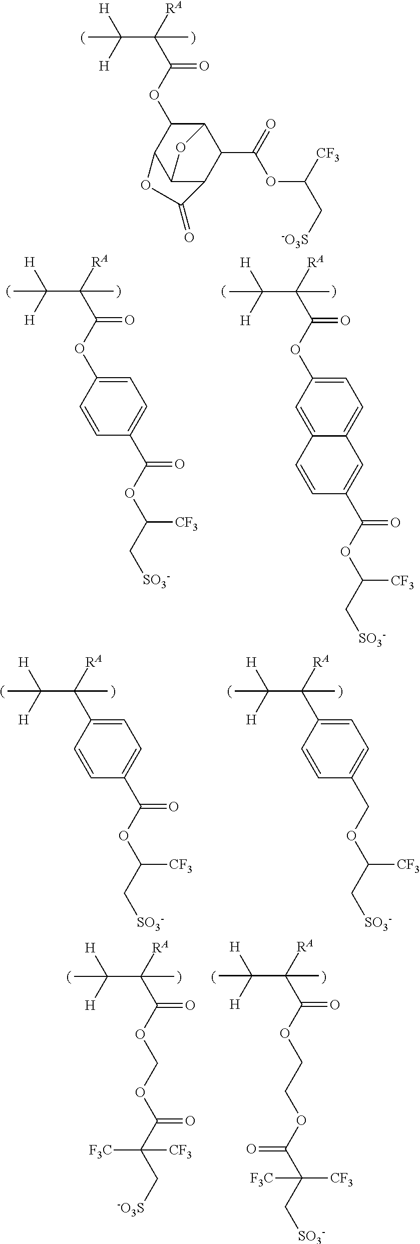

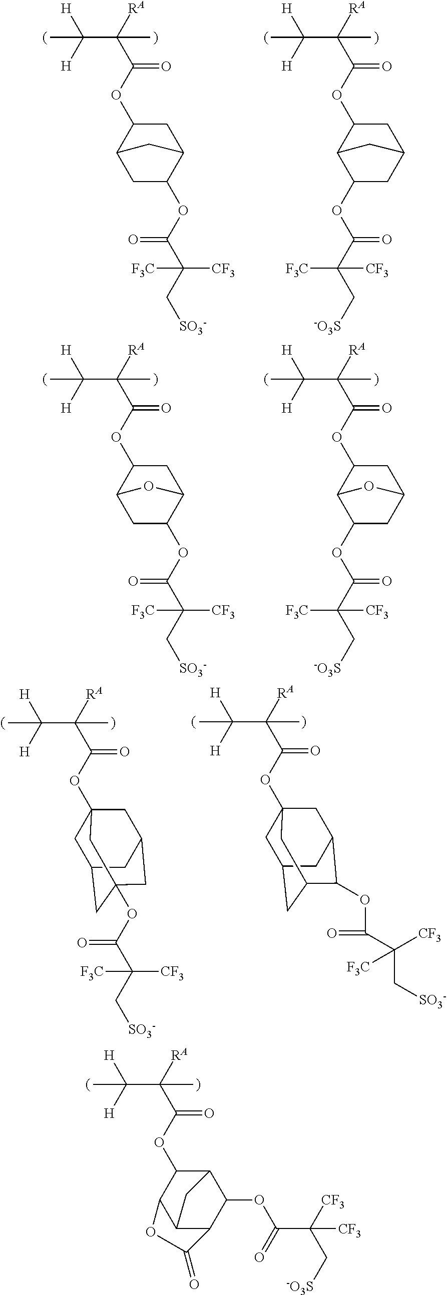

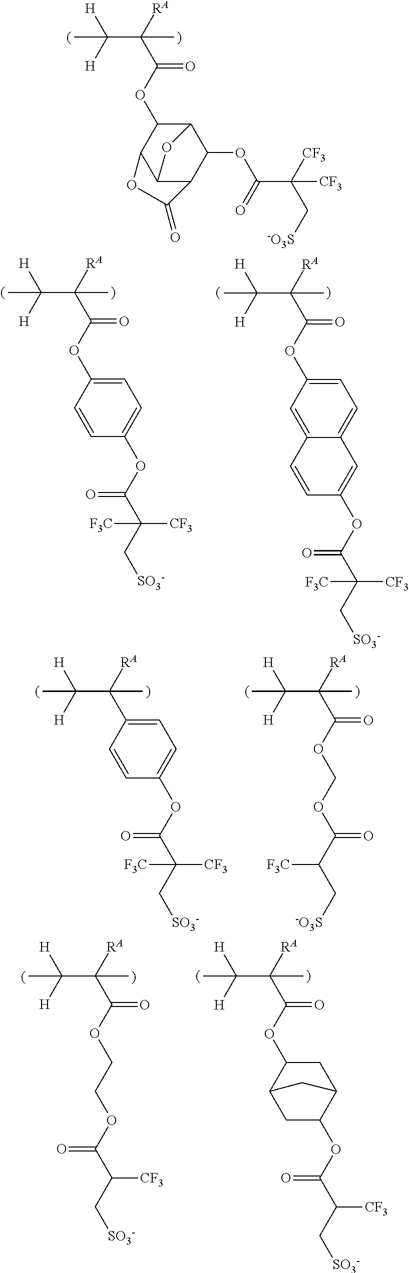

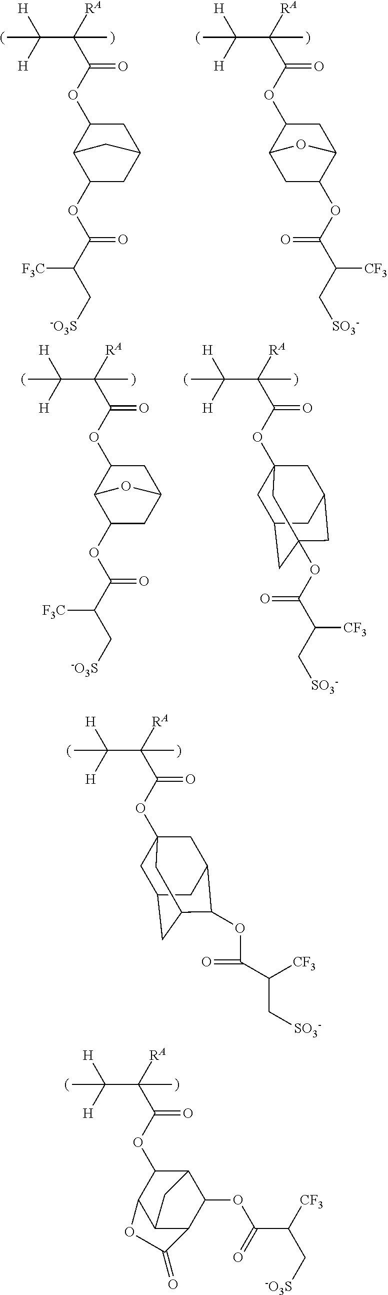

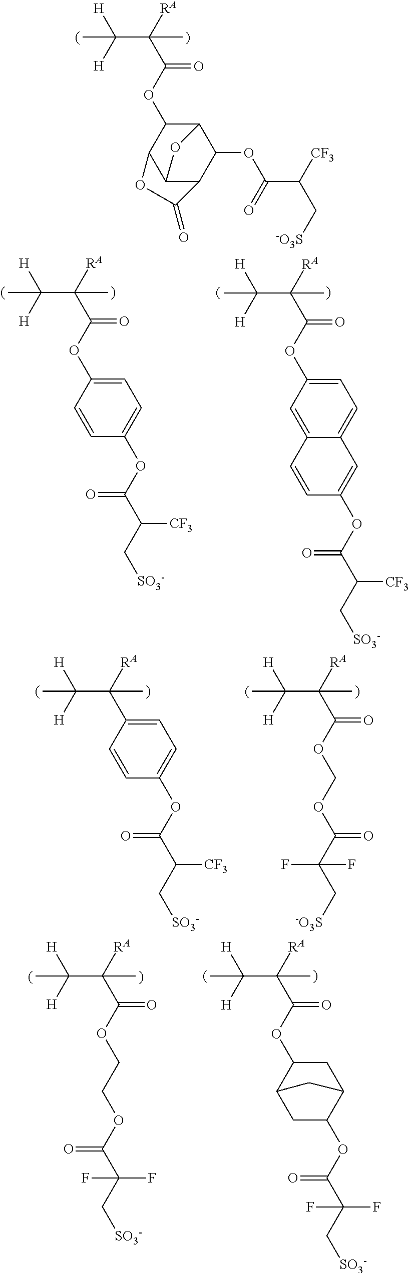

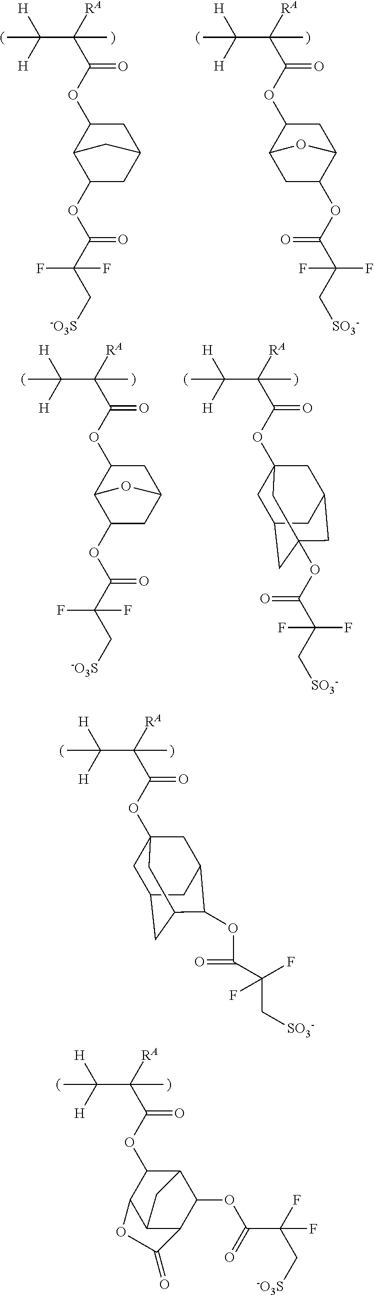

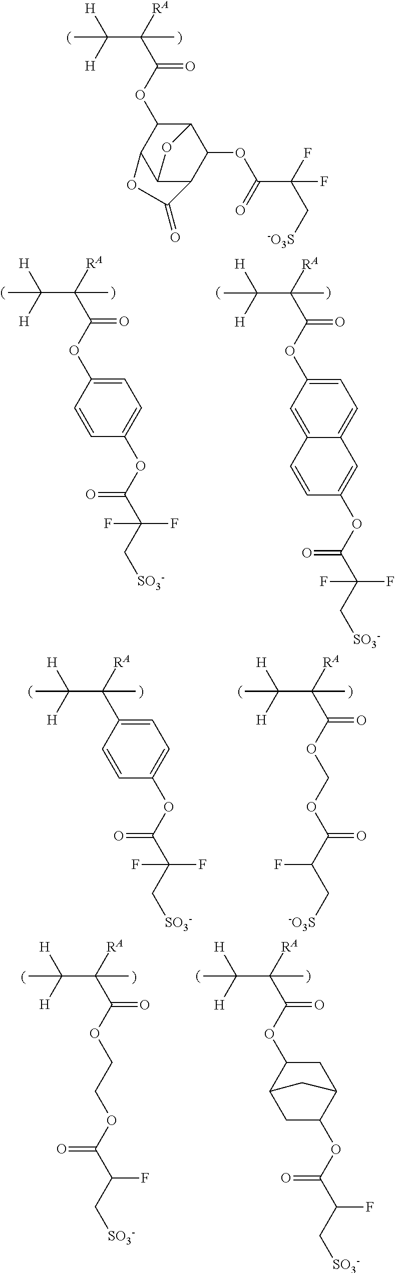

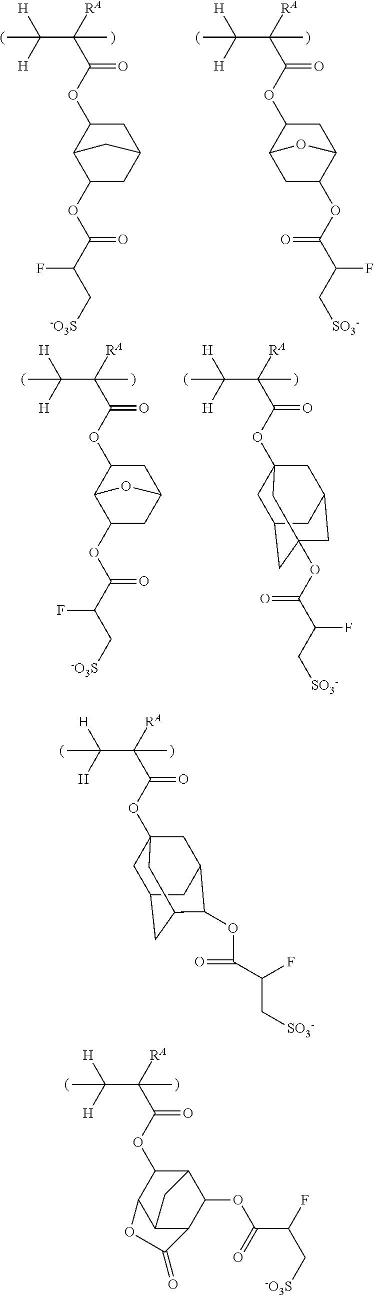

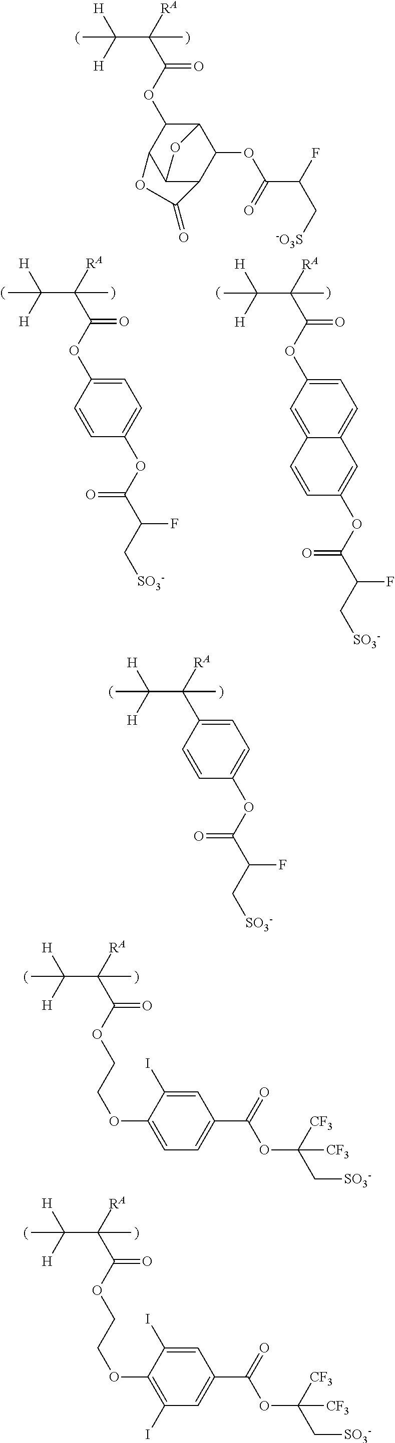

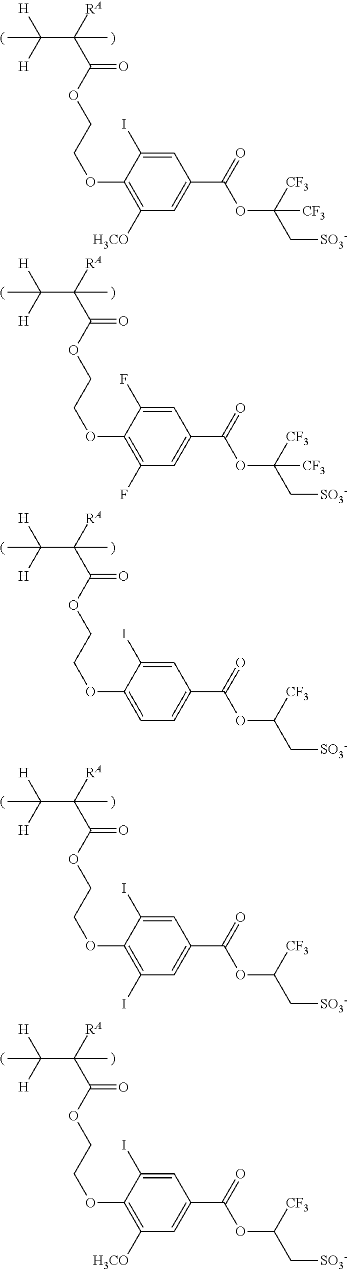

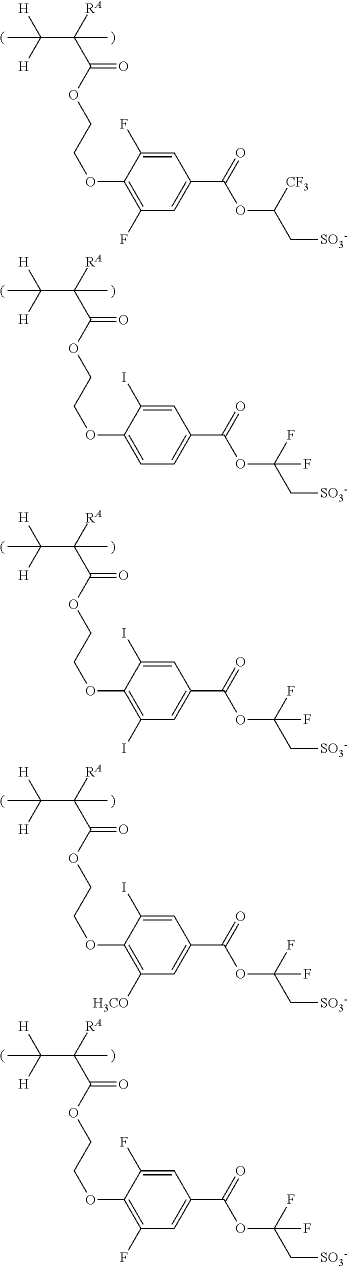

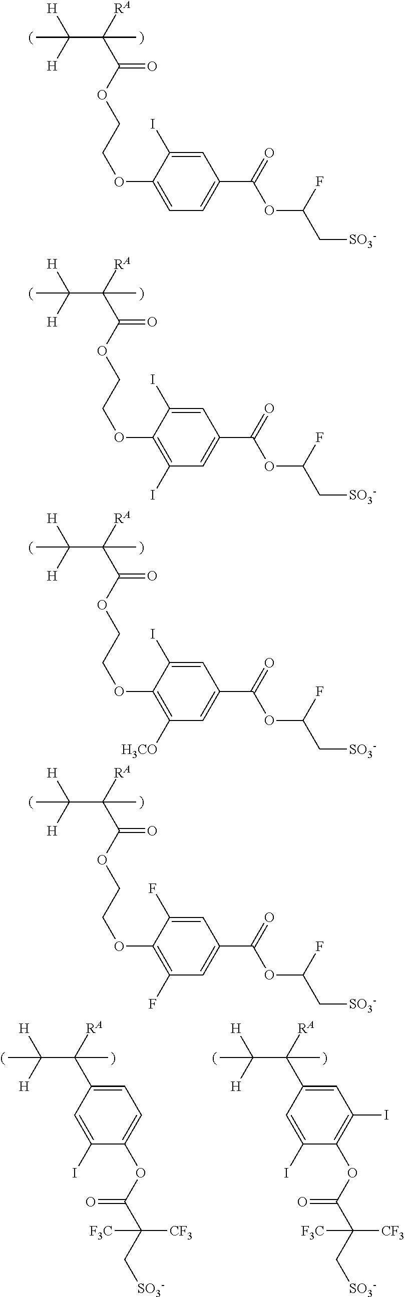

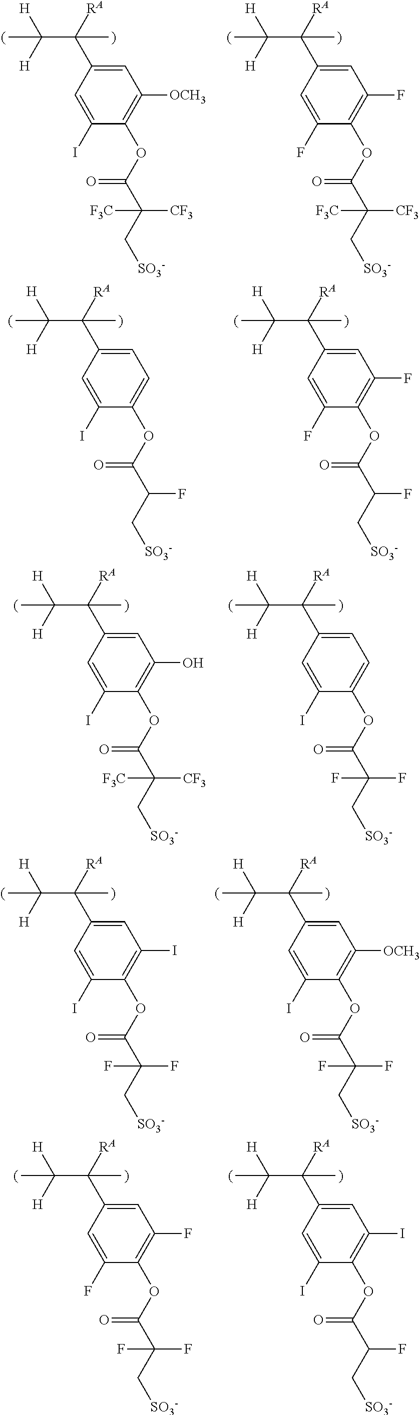

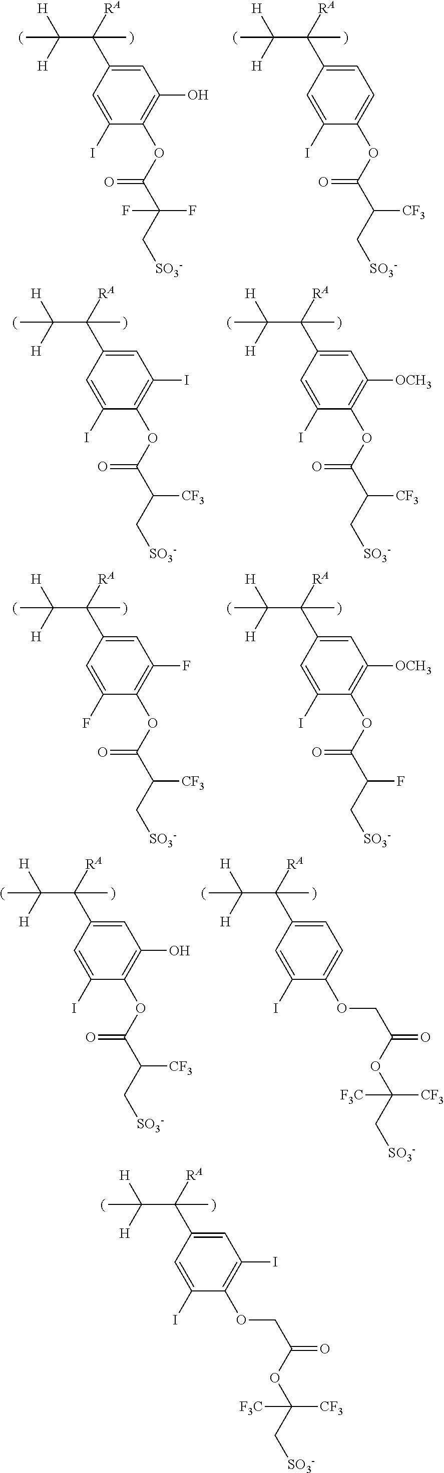

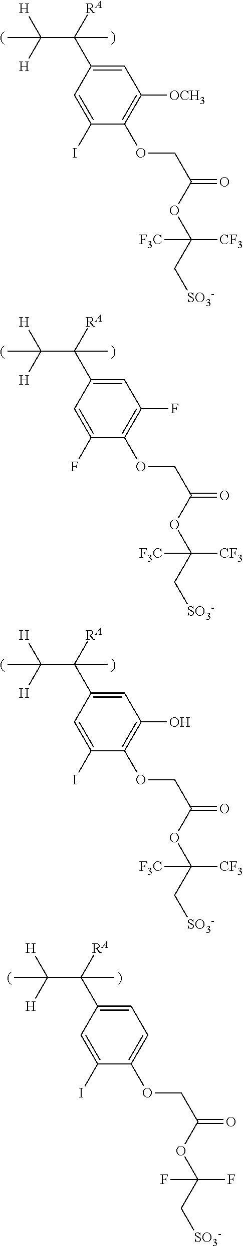

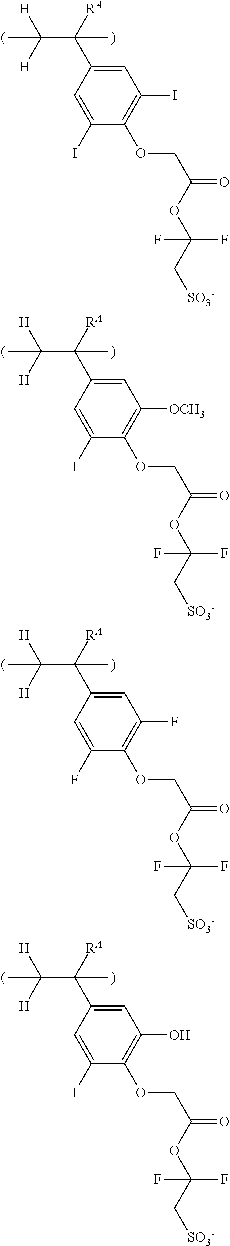

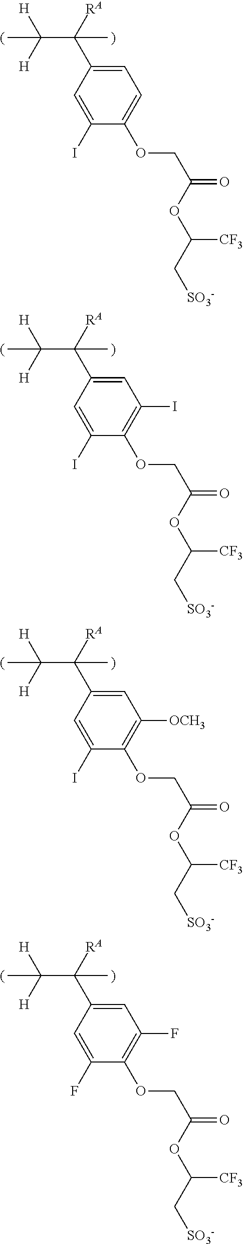

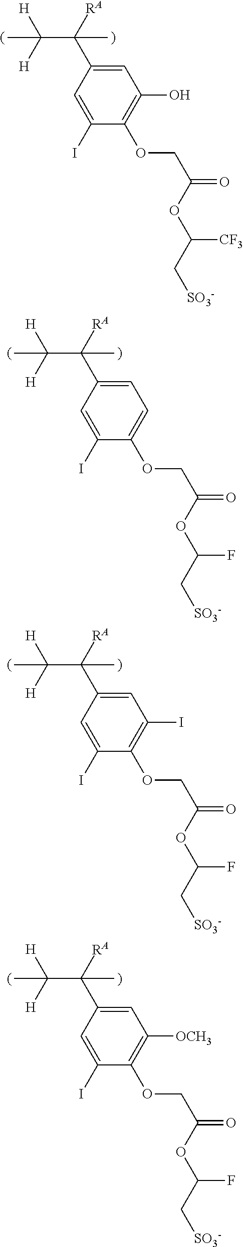

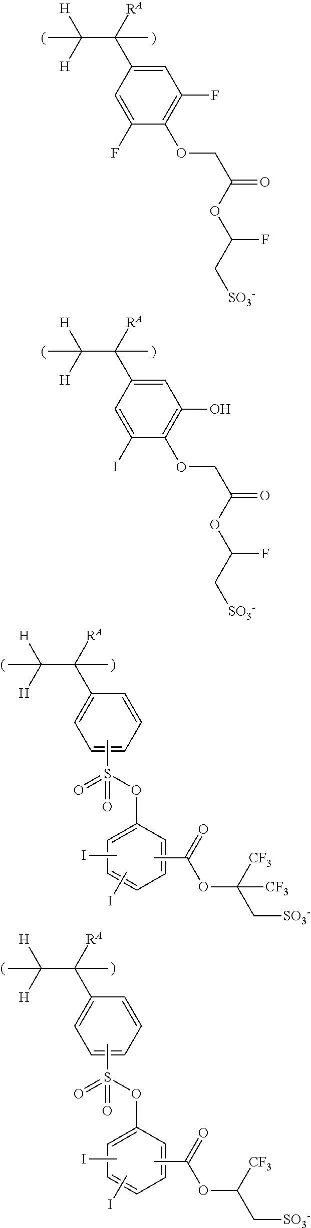

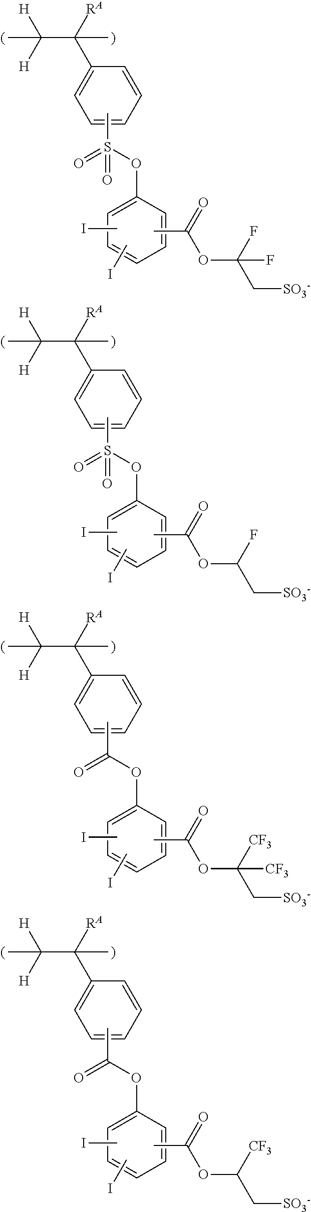

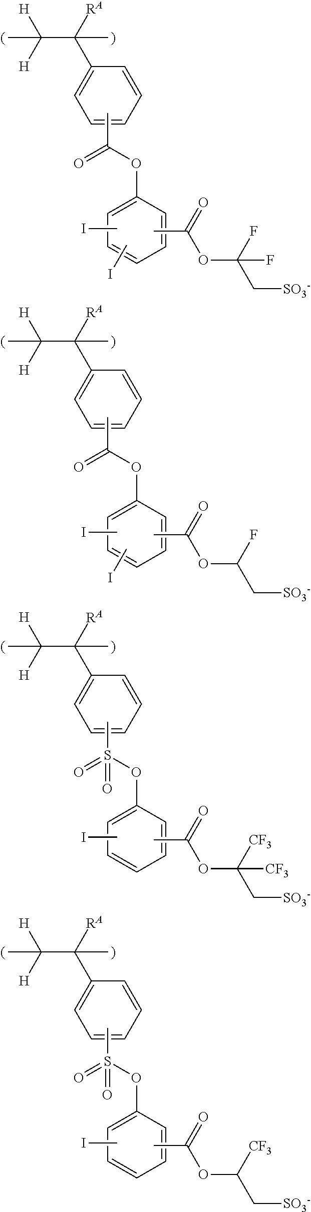

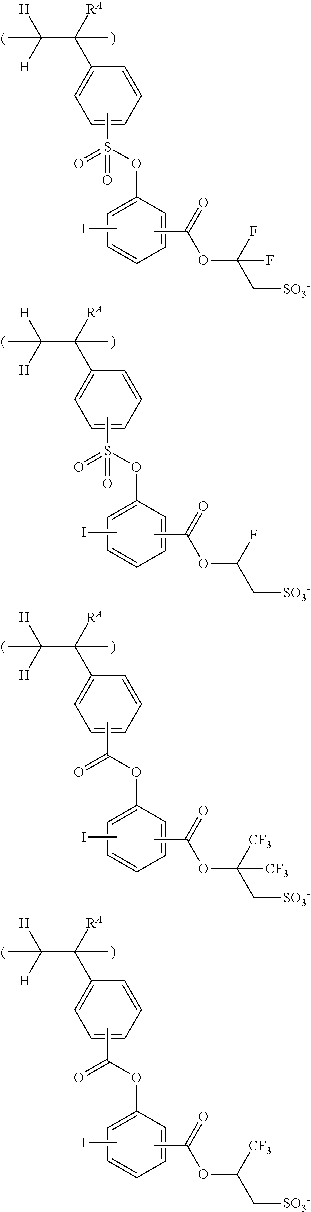

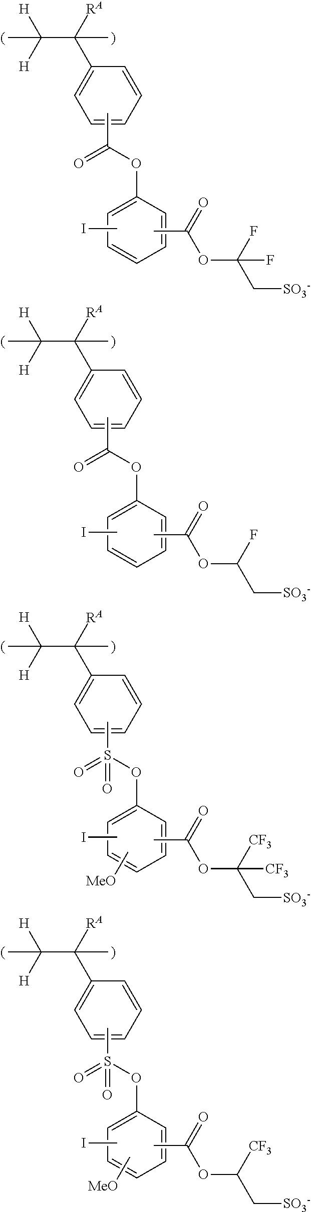

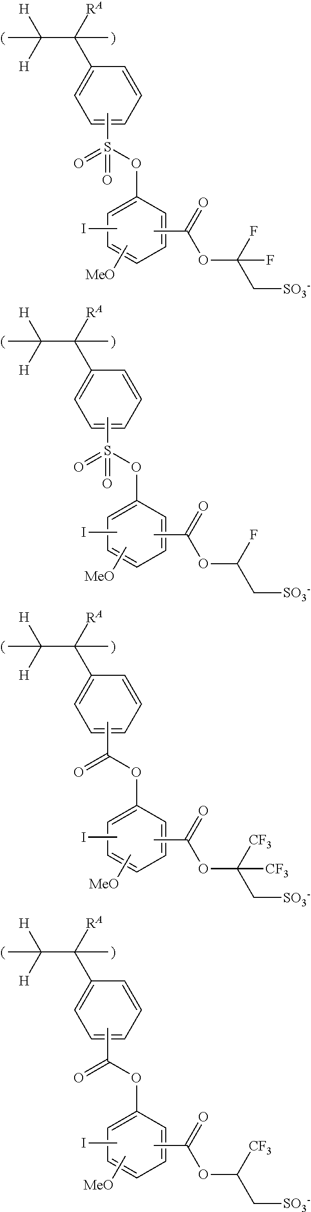

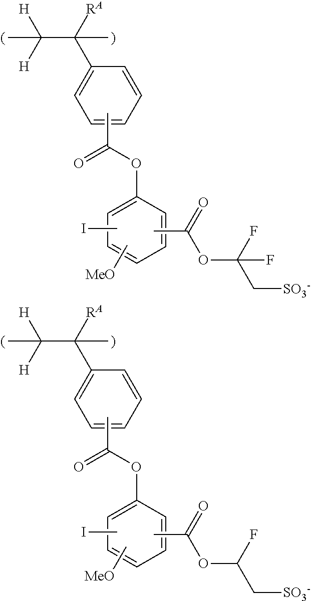

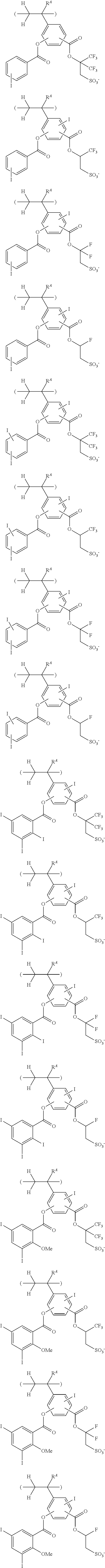

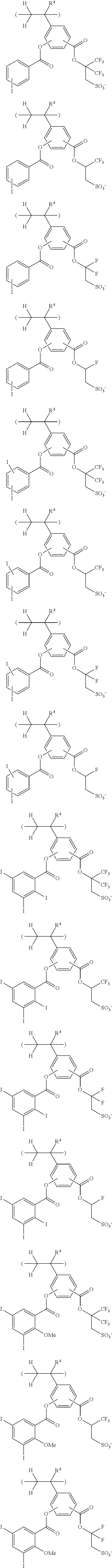

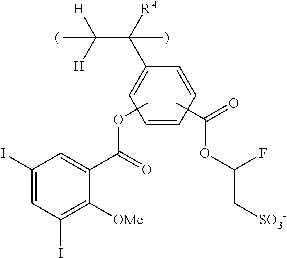

Examples of the anion in repeat unit (c4) are shown below, but not limited thereto. R^{A} is as defined above.

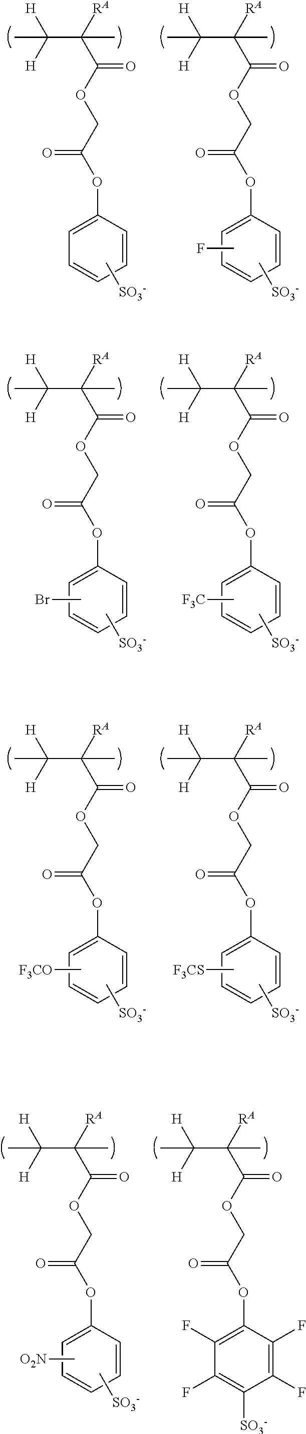

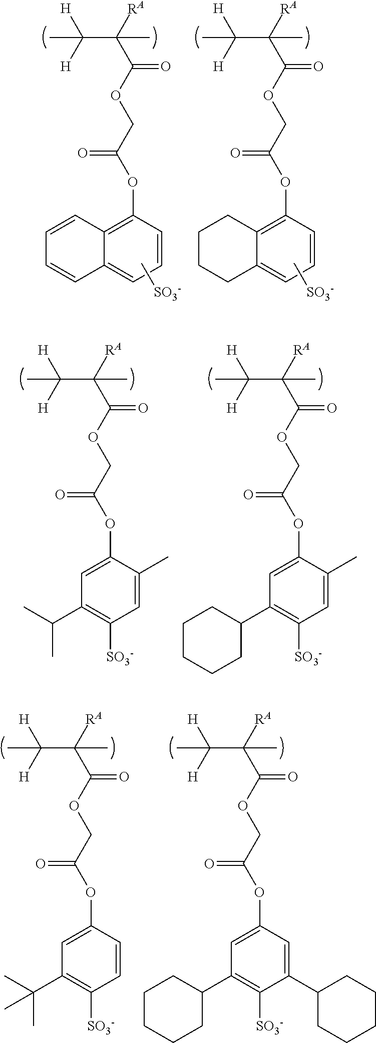

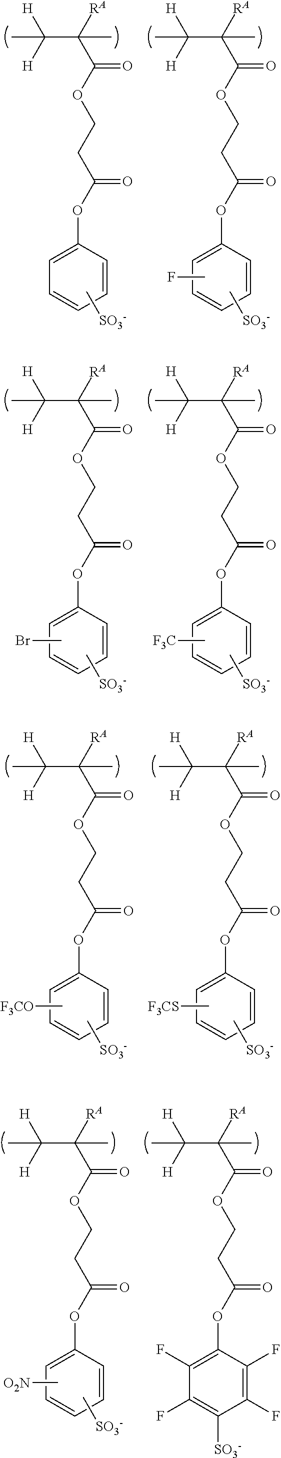

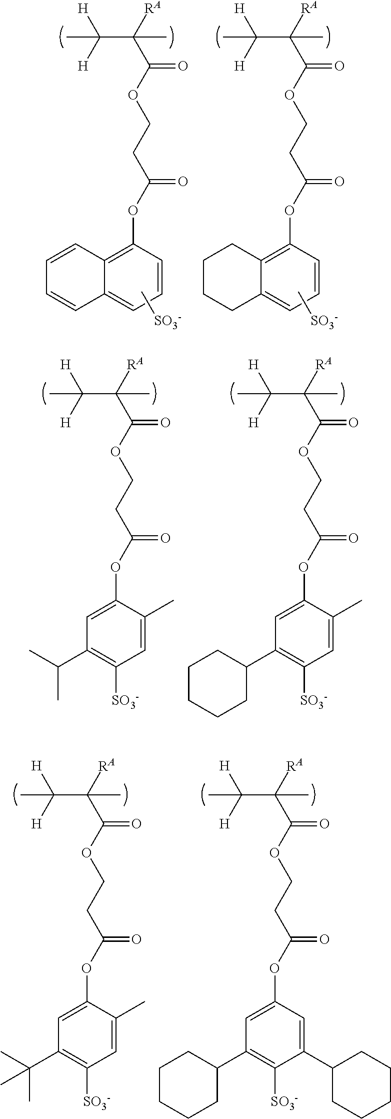

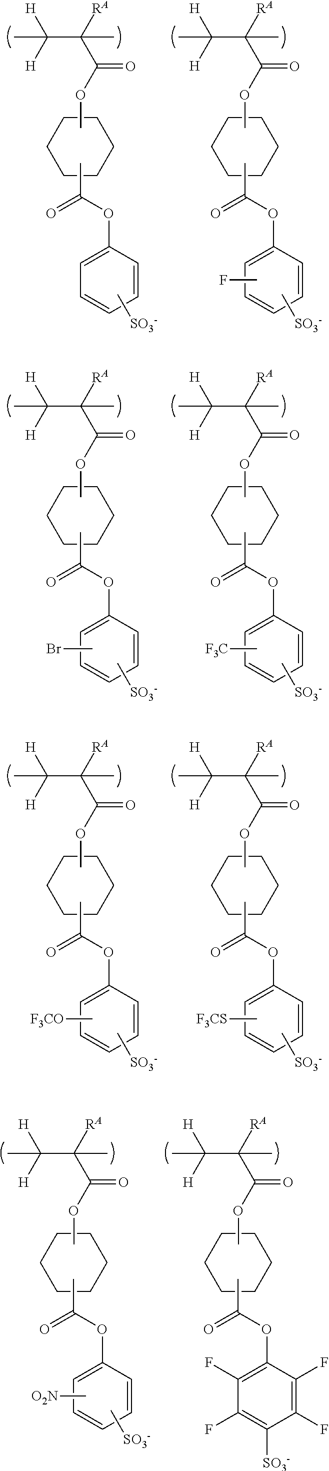

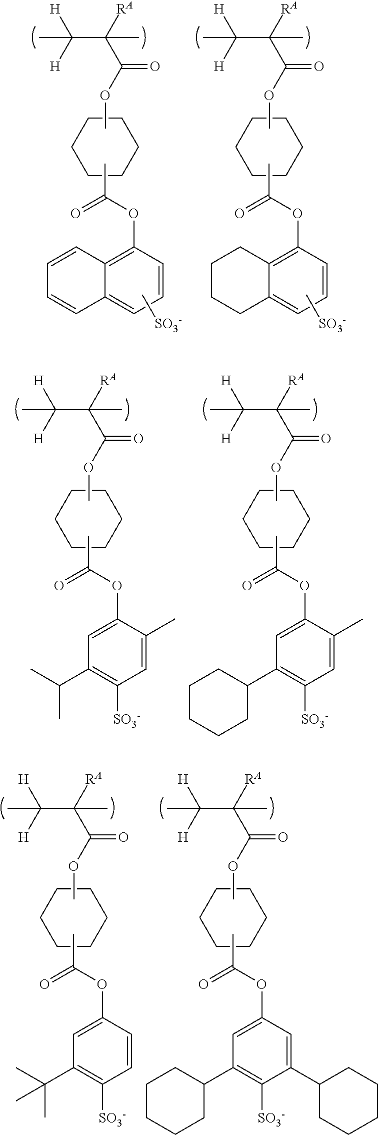

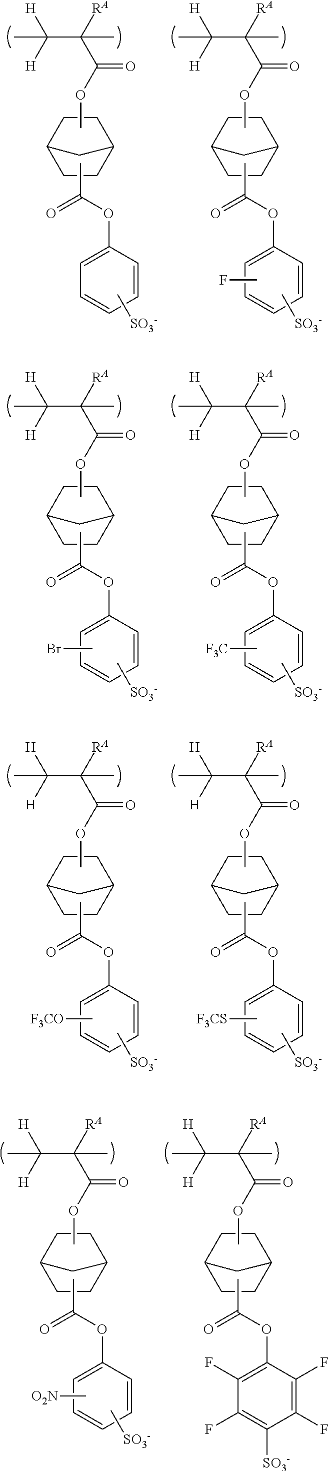

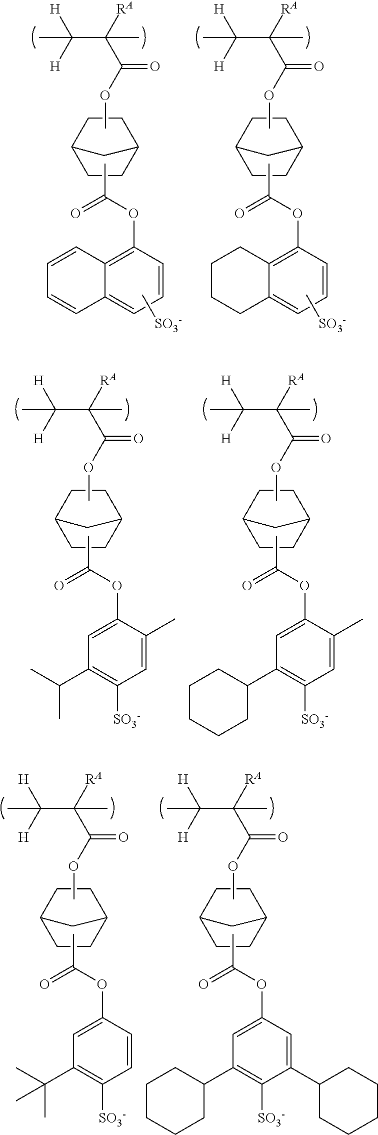

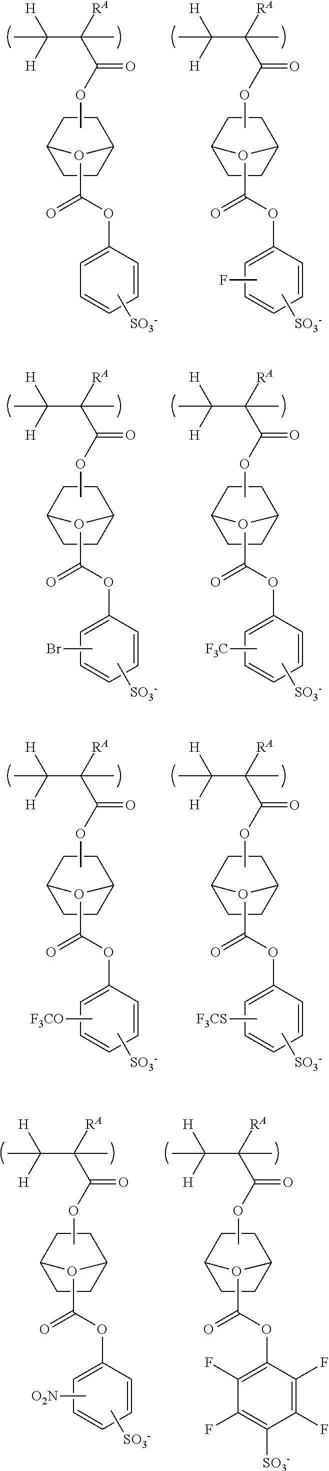

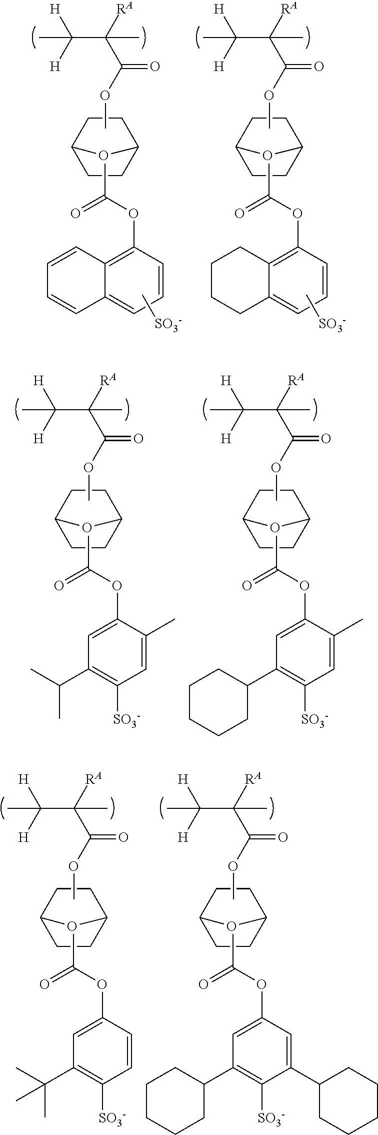

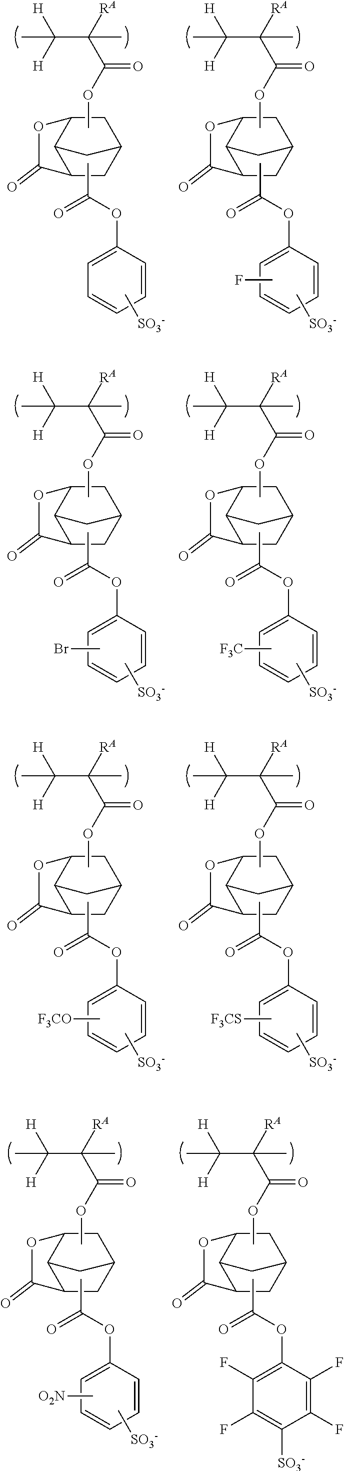

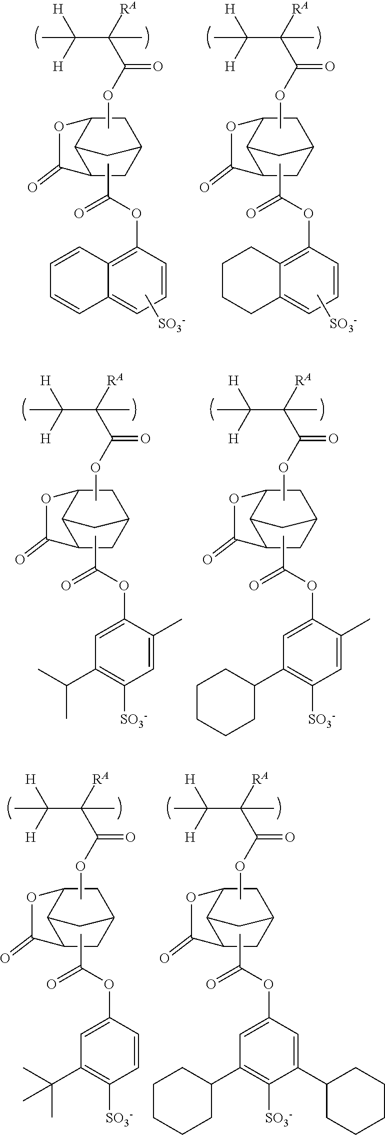

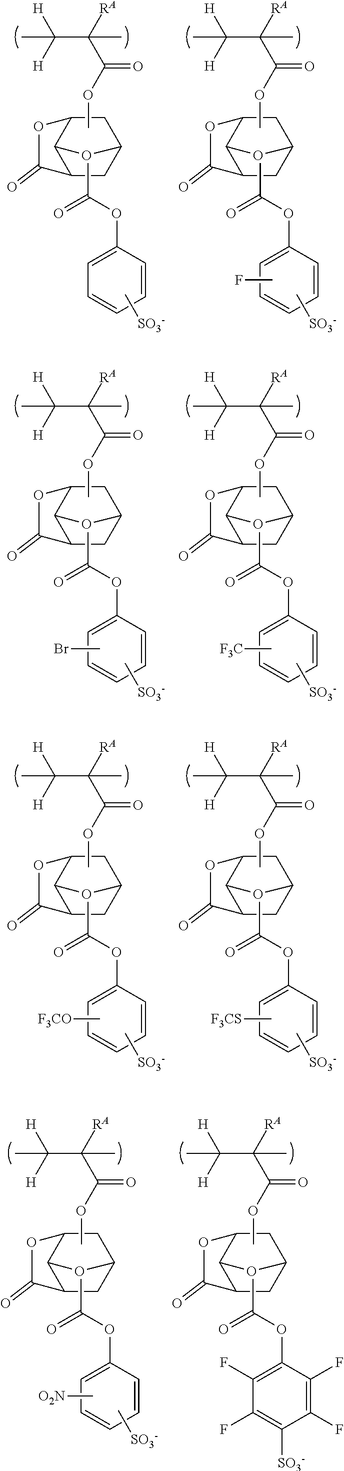

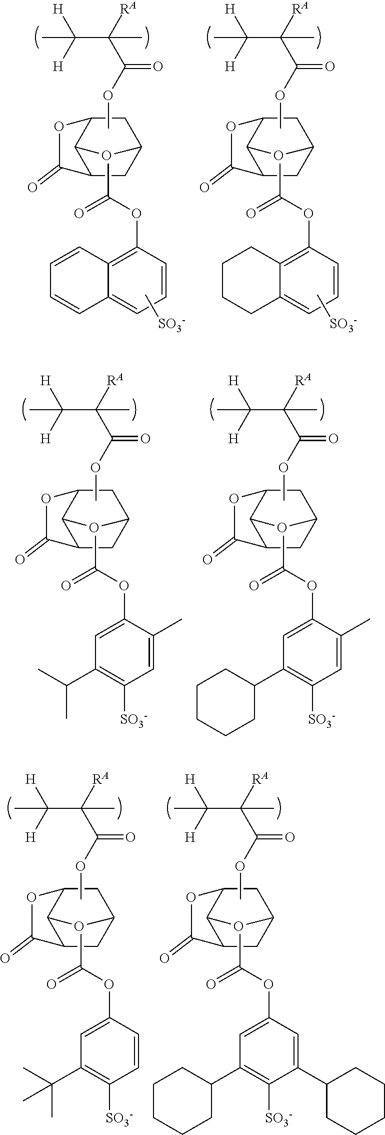

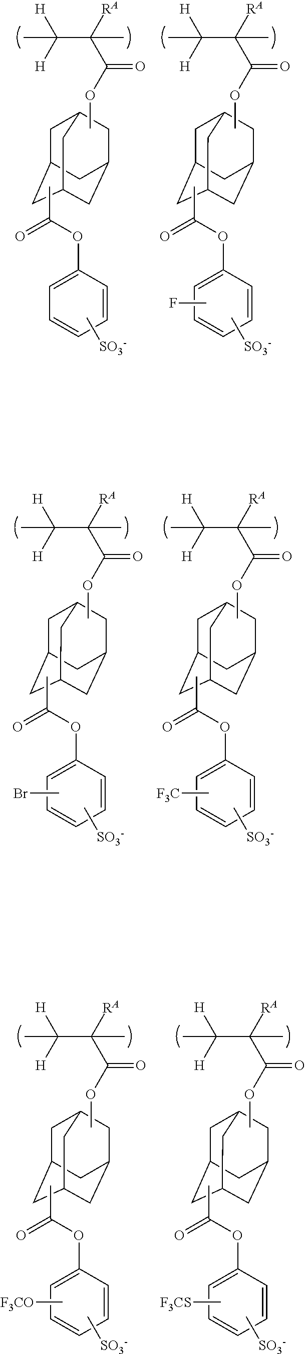

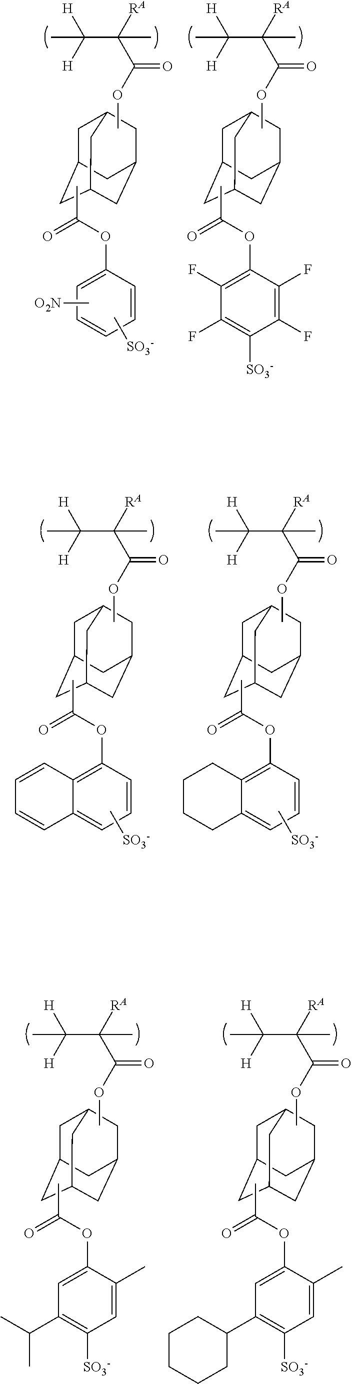

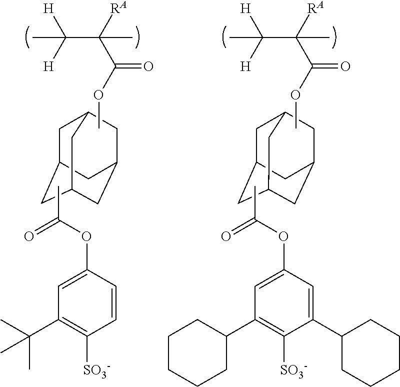

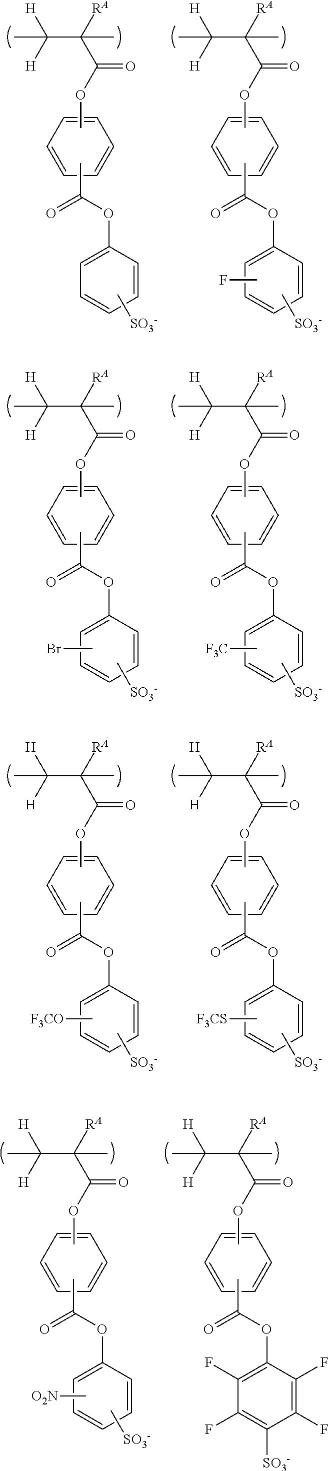

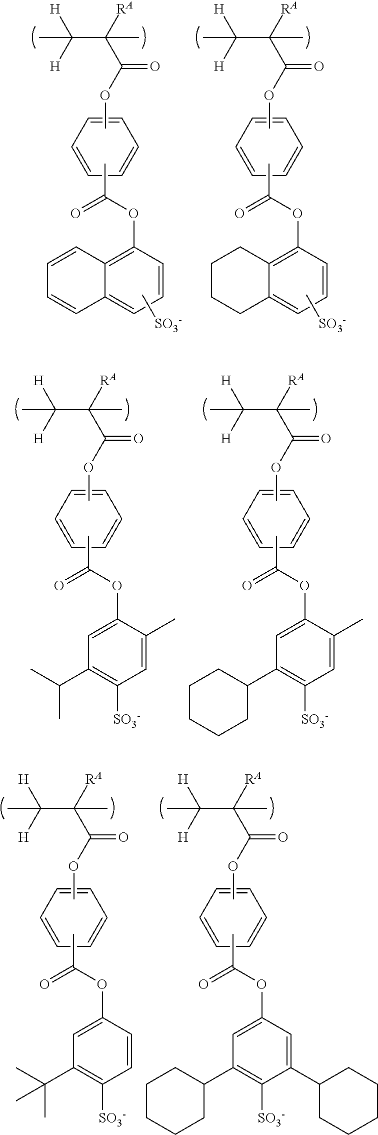

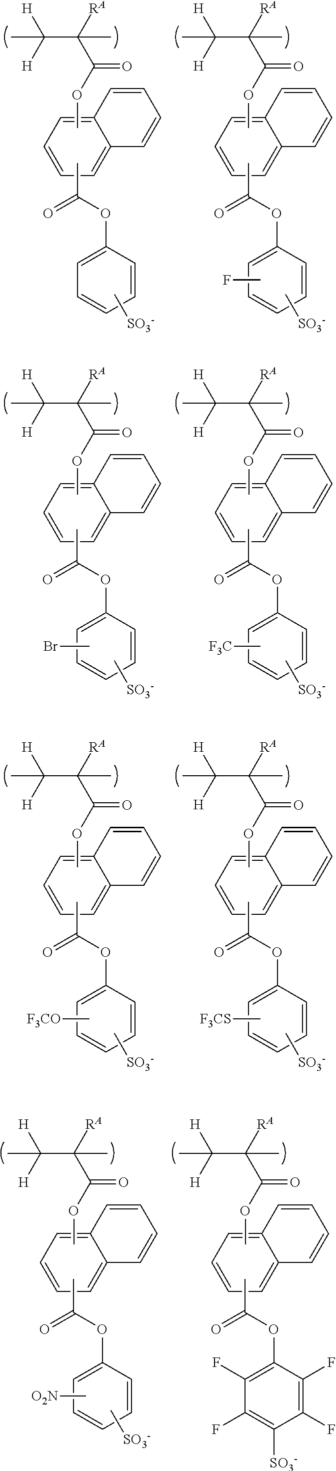

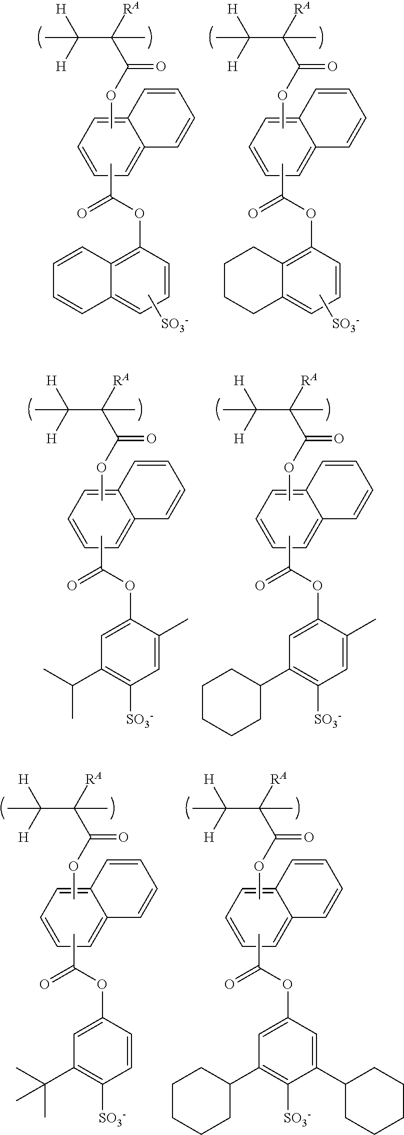

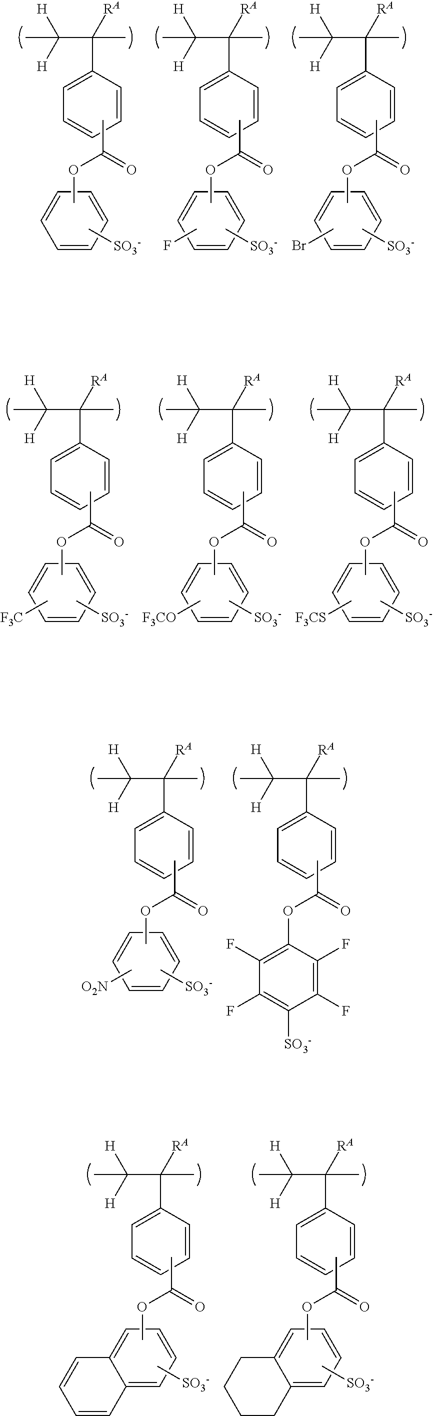

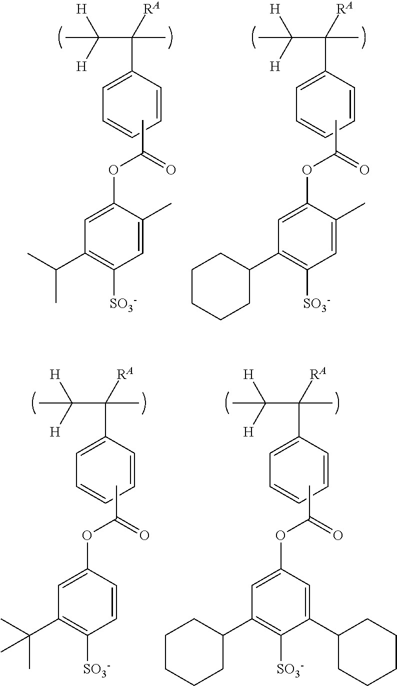

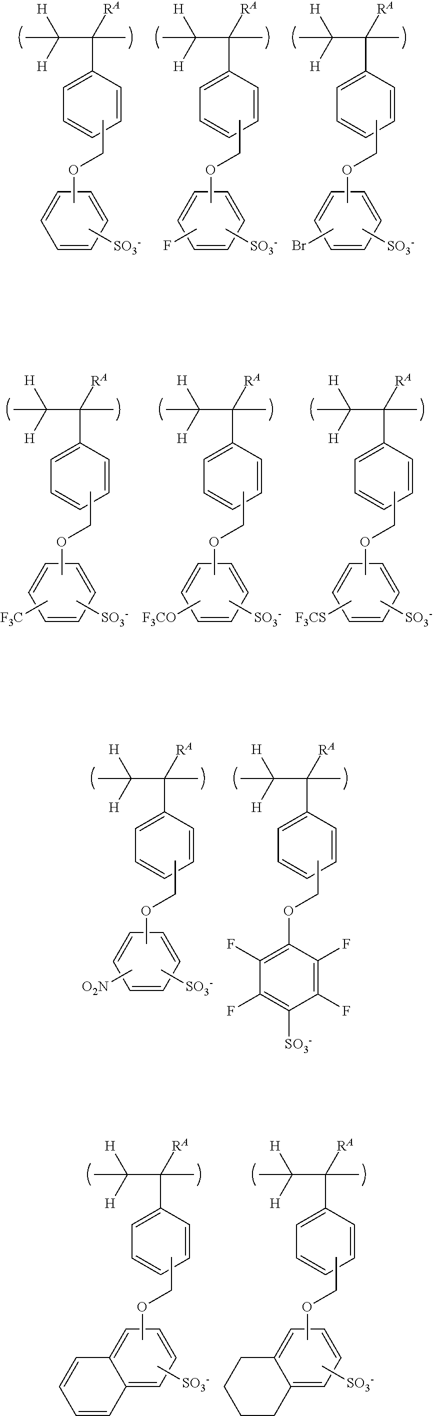

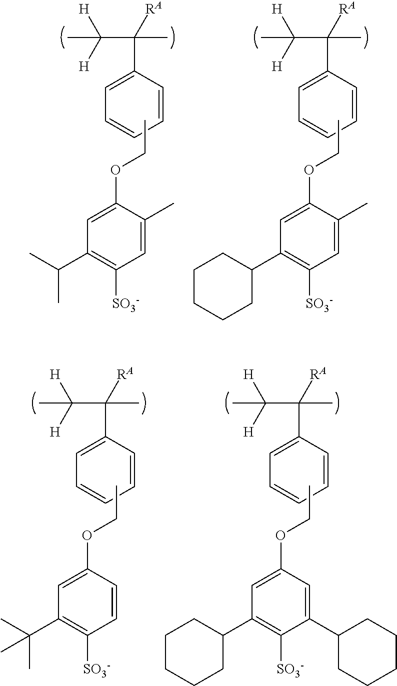

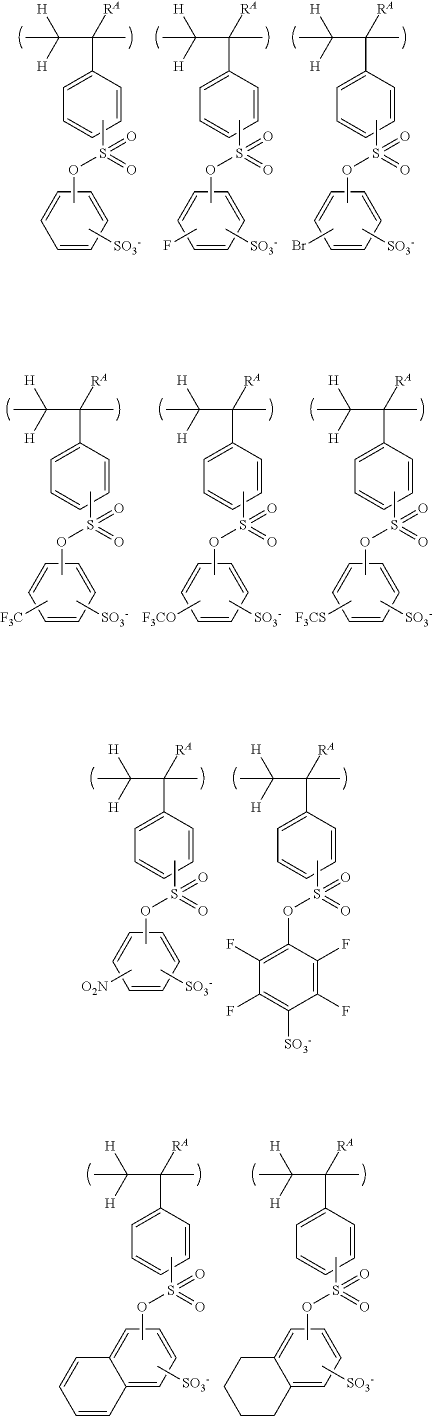

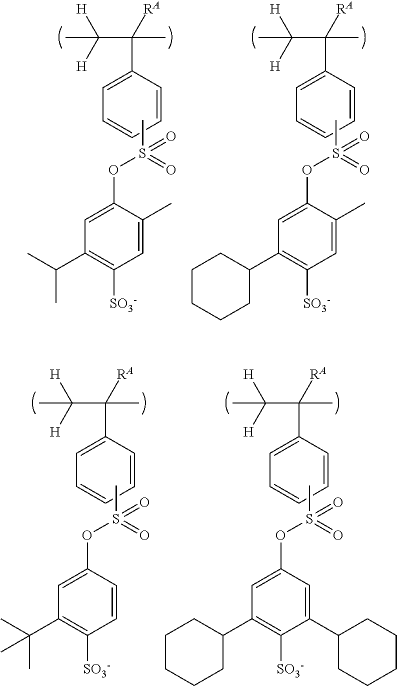

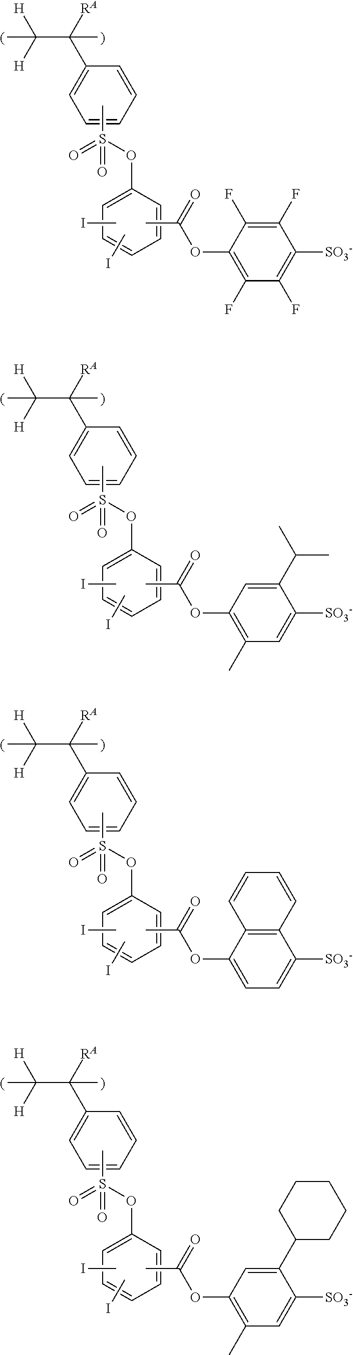

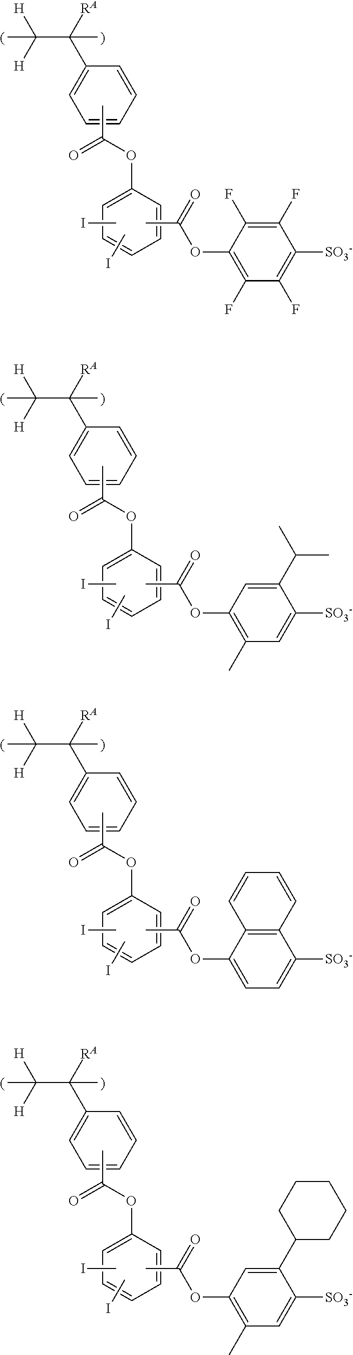

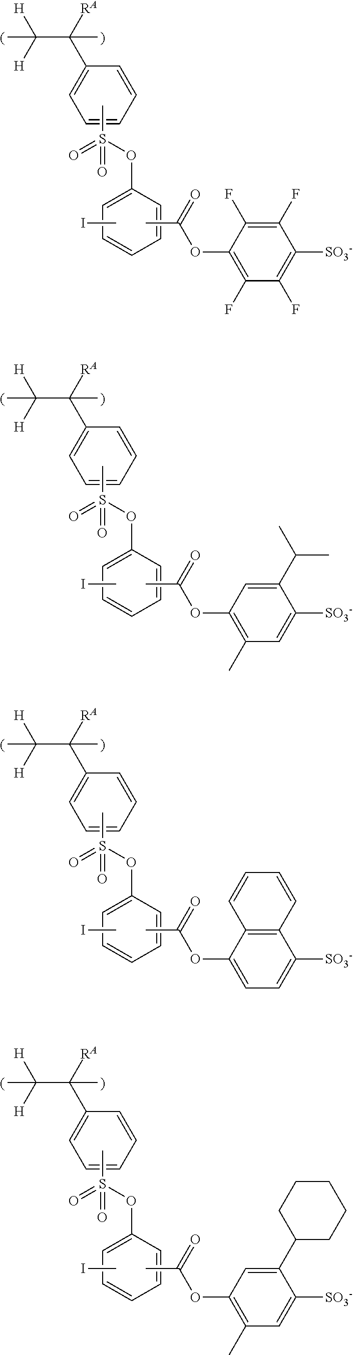

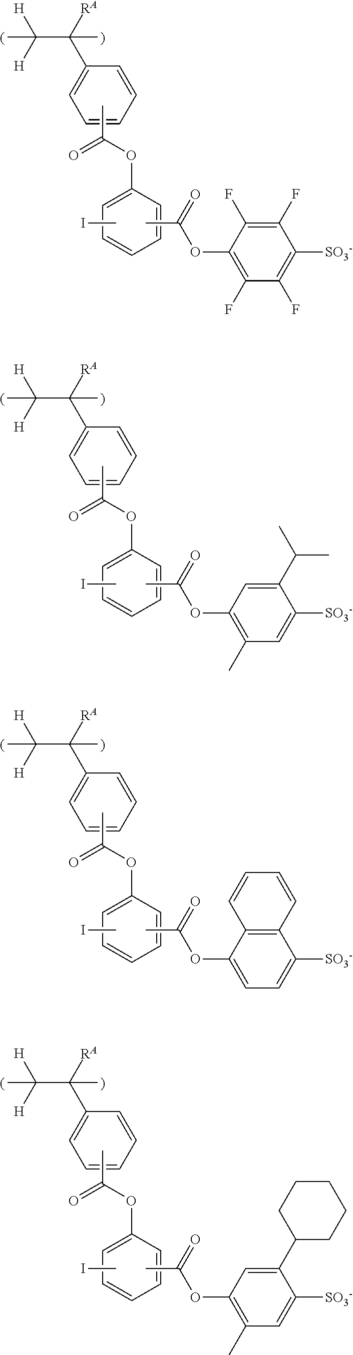

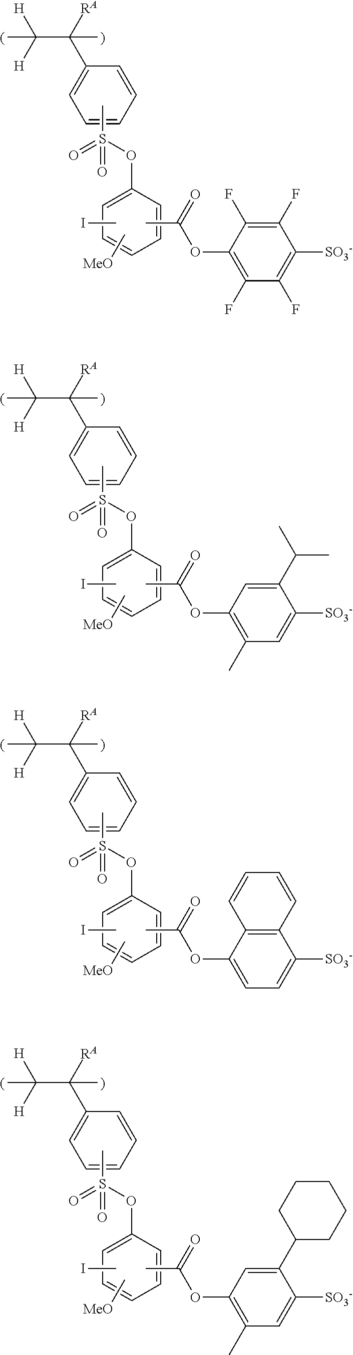

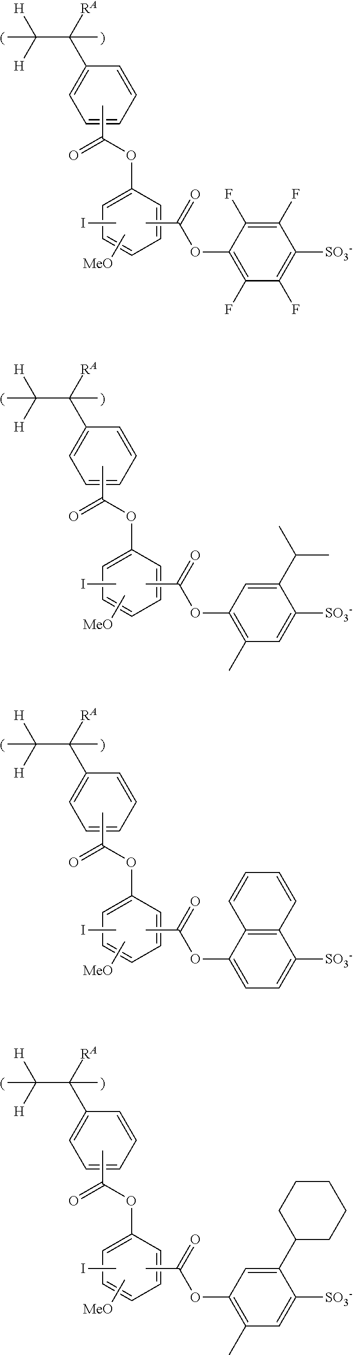

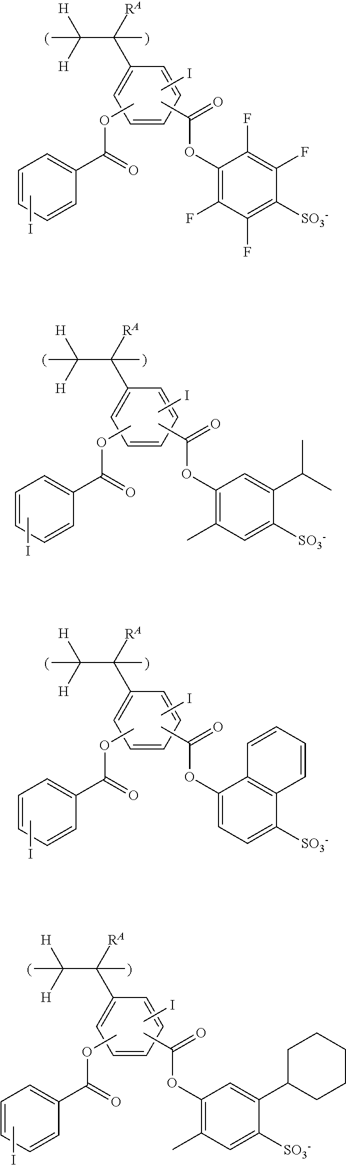

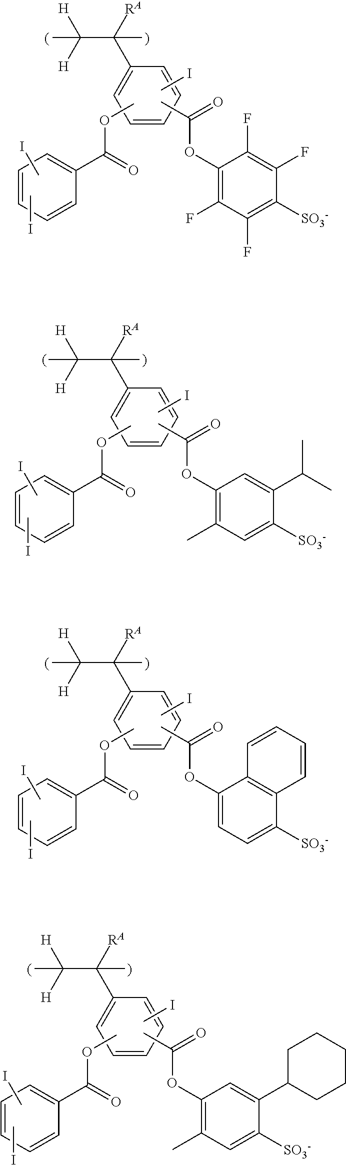

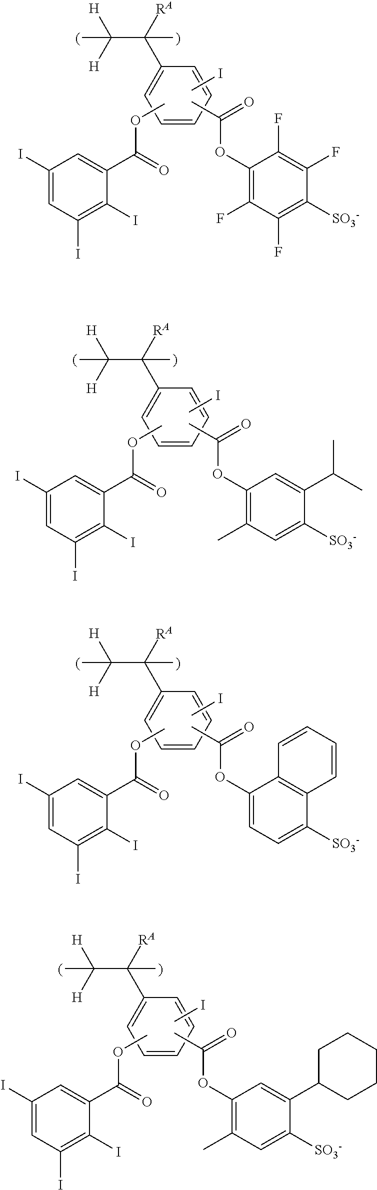

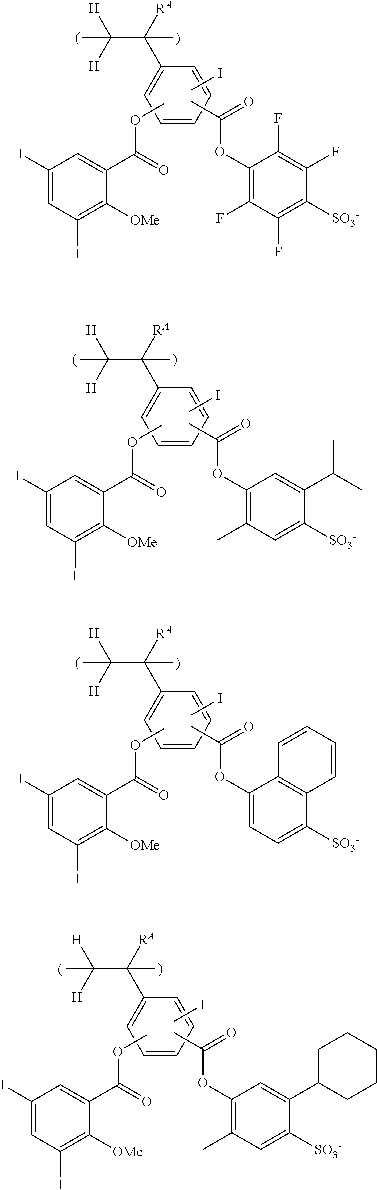

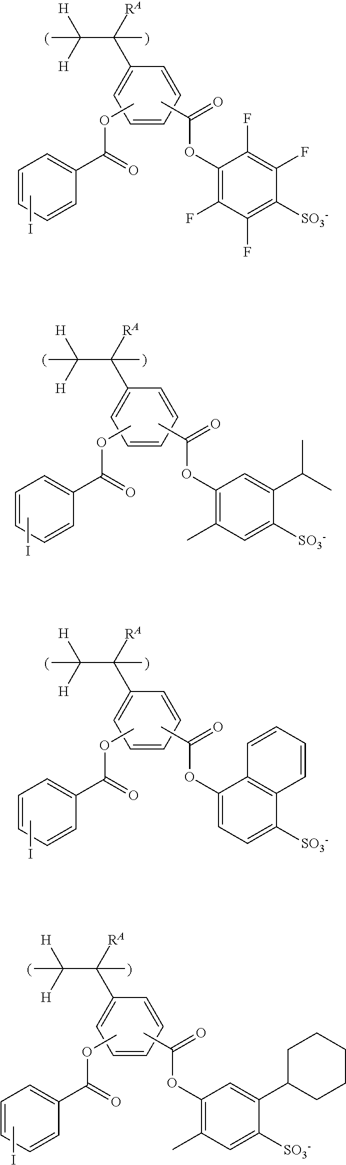

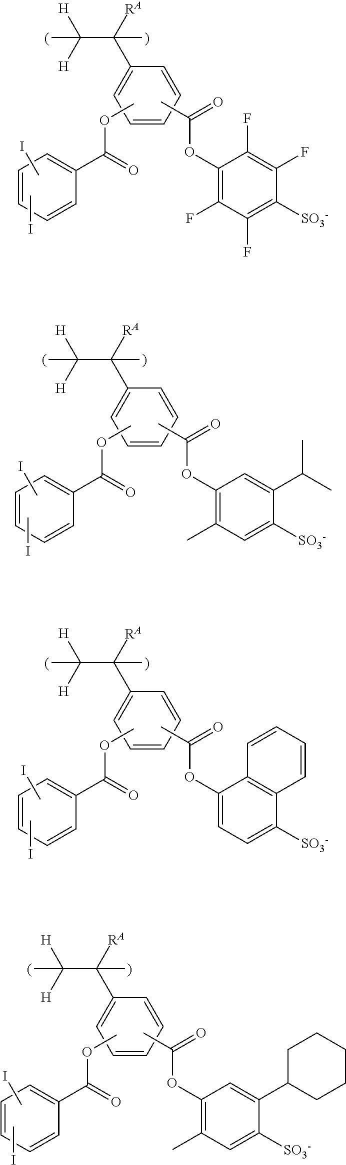

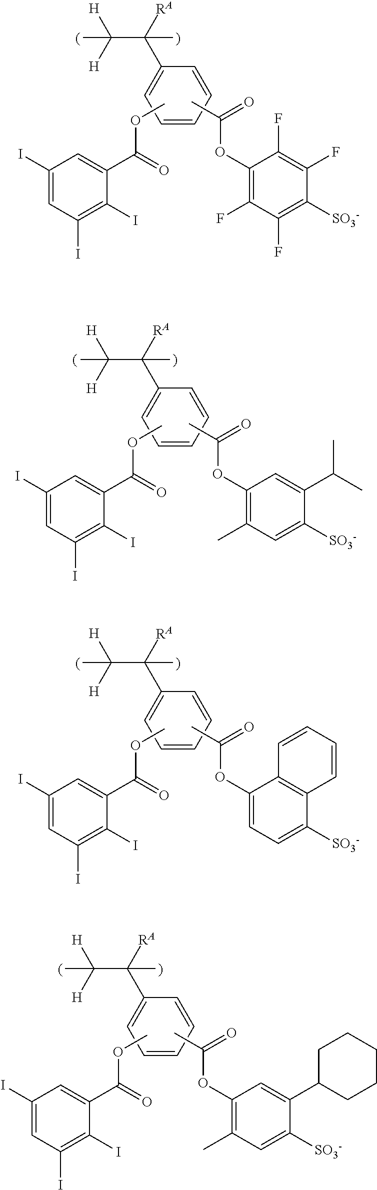

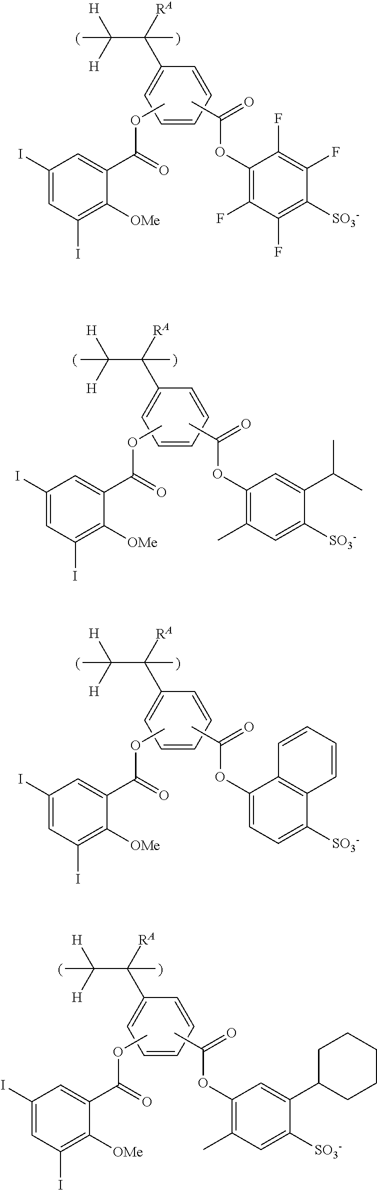

Examples of the anion in repeat unit (c5) are shown below, but not limited thereto. R^{A} is as defined above.

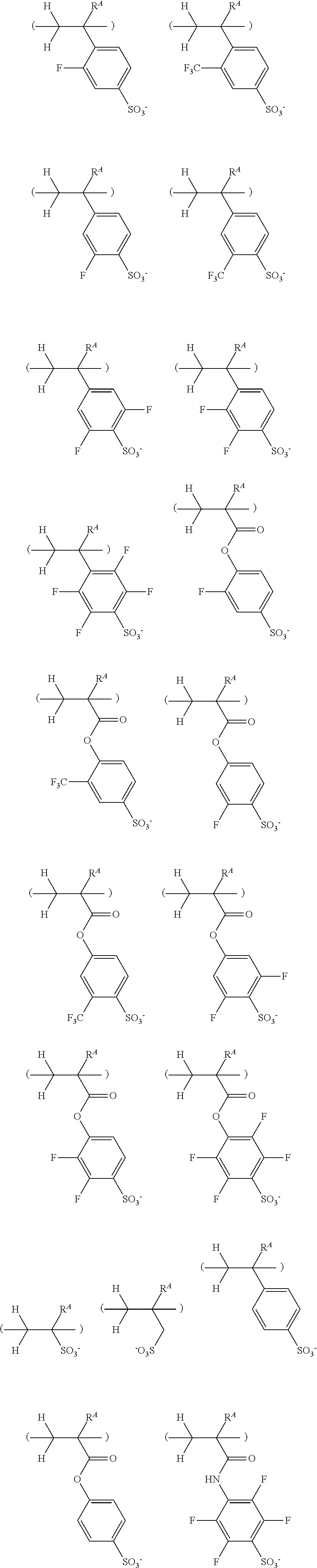

In formulae (c2) to (c5), Z⁺ is an onium cation. The preferred onium cation Z⁺ is a sulfonium cation having the formula (Z-1) or iodonium cation having the formula (Z-2).

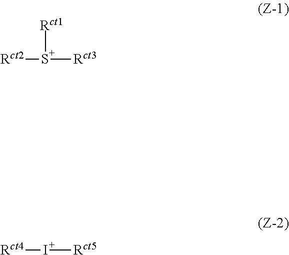

In formulae (Z-1) and (Z-2), R^{ct1} to R^{ct5} are each independently halogen or a C₁-C₃₀ hydrocarbyl group which may contain a heteroatom.

Suitable halogen atoms include fluorine, chlorine, bromine, and iodine.

The hydrocarbyl group may be saturated or unsaturated and straight, branched or cyclic. Examples thereof include C₁-C₃₀ alkyl groups such as methyl, ethyl, n-propyl, isopropyl, n-butyl, isobutyl, sec-butyl, and tert-butyl; C₃-C₃₀ cyclic saturated hydrocarbyl groups such as cyclopropyl, cyclopentyl, cyclohexyl, cylopropylmethyl, 4-methylcyclohexyl, cyclohexylmethyl, norbornyl, and adamantyl; C₂-C₃₀ alkenyl groups such as vinyl, 1-propenyl, 2-propenyl, butenyl, and hexenyl; C₃-C₃₀ cyclic unsaturated hydrocarbyl groups such as cyclohexenyl; C₆-C₃₀ aryl groups such as phenyl, naphthyl, and thienyl; C₇-C₃₀ aralkyl groups such as benzyl, 1-phenylethyl, and 2-phenylethyl, and combinations thereof. Of these, aryl groups are preferred. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, nitro moiety, carbonyl moiety, ether bond, ester bond, sulfonic ester bond, carbonate bond, lactone ring, sultone ring, carboxylic anhydride (—C(═O)—O—C(═O)—) or haloalkyl moiety.

Also, R^{ct1} and R^{ct2} may bond together to form a ring with the sulfur atom to which they are attached. Exemplary structures of the ring are shown below.

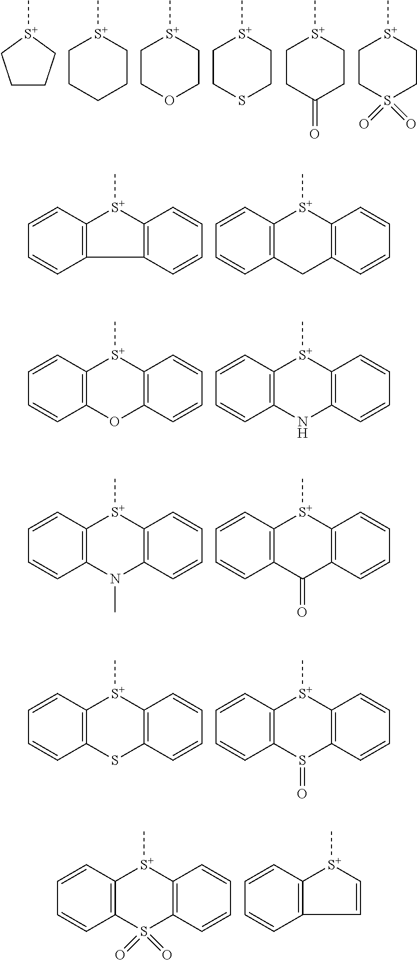

Herein the broken line designates a point of attachment to R^{ct3}.

Examples of the sulfonium cation having formula (Z-1) include those described in JP-A 2024-003744, paragraphs [0102]-[0125], but are not limited thereto. Examples of the iodonium cation having formula (Z-2) include those described in JP-A 2024-000259, paragraph [0181], but are not limited thereto.

A sulfonium cation having the formula (Z-3) is also preferred as the onium cation Z⁺.

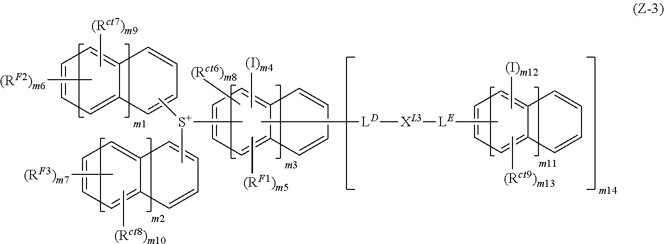

In formula (Z-3), m1 is 0 or 1. The relevant structure is a benzene ring when m1=0, and a naphthalene ring when m1=1. The benzene ring corresponding to m1=0 is preferred from the aspect of solvent solubility. The subscript m2 is 0 or 1. The relevant structure is a benzene ring when m2=0, and a naphthalene ring when m2=1. The benzene ring corresponding to m2=0 is preferred from the aspect of solvent solubility. The subscript m3 is 0 or 1. The relevant structure is a benzene ring when m3=0, and a naphthalene ring when m3=1. The benzene ring corresponding to m3=0 is preferred from the aspect of solvent solubility.

In formula (Z-3), m4 is 0, 1, 2, 3 or 4. As the number of iodine atoms in the cation structure increases, the compound becomes more absorptive to EUV, but so poor in solvent solubility that it may precipitate in a resist composition. For this reason, m4 is preferably 0, 1, 2 or 3, more preferably 0, 1 or 2.

In formula (Z-3), m5 is 0, 1, 2, 3 or 4. From the aspect of reactant availability, m5 is preferably 0, 1, 2 or 3, more preferably 0, 1 or 2. The subscript m6 is 0, 1, 2, 3, 4, 5 or 6. From the aspect of reactant availability, m6 is preferably 0, 1, 2 or 3, more preferably 0, 1 or 2. The subscript m7 is 0, 1, 2, 3, 4, 5 or 6. From the aspect of reactant availability, m7 is preferably 0, 1, 2 or 3, more preferably 0, 1 or 2.

In formula (Z-3), m8 is 0, 1 or 2. From the aspect of reactant availability, m8 is preferably 0 or 1. The subscript m9 is 0, 1 or 2. From the aspect of reactant availability, m9 is preferably 0 or 1. The subscript m10 is 0, 1 or 2. From the aspect of reactant availability, m10 is preferably 0 or 1.

In formula (Z-3), m11 is 0 or 1. The relevant structure is a benzene ring when m11=0, and a naphthalene ring when m11=1. The benzene ring corresponding to m11=0 is preferred from the aspect of solvent solubility.

In formula (Z-3), m12 is 0, 1, 2, 3 or 4. As the number of iodine atoms in the cation structure increases, the compound becomes more absorptive to EUV, but so poor in solvent solubility that it may precipitate in a resist composition. For this reason, m12 is preferably 0, 1, 2 or 3, more preferably 0, 1 or 2.

In formula (Z-3), m13 is 0, 1 or 2. From the aspect of reactant availability, m13 is preferably 0 or 1. The subscript m14 is 0, 1 or 2. From the aspect of synthesis, m14 is preferably 0 or 1.

The subscripts m1 to m14 are in the range: 0 m6+m9≤4 when m1=0, 0≤m6+m9≤6 when m1=1; 0≤m7+m10≤4 when m2=0, 0≤m7+m10≤6 when m2=1; 1≤m4+m5+m8+m14≤4 when m3=0, 1≤m4+m5+m8+m14≤6 when m3=1; 0≤m12+m13≤4 when m11=0, 0≤m12+m13≤6 when m11=1; and m4+m12≥1.

In formula (Z-3), R^{F1} to R^{F3} are each independently fluorine, a C₁-C₆ fluorinated saturated hydrocarbyl group, C₁-C₆ fluorinated saturated hydrocarbyloxy group, or C₁-C₆ fluorinated saturated hydrocarbylthio group. Of these, trifluoromethyl, trifluoromethoxy, and trifluorothiomethoxy are preferred. A plurality of R^{F}1 may be identical or different when m5 is 2, 3 or 4, a plurality of R^{F2} may be identical or different when m6 is 2, 3, 4, 5 or 6, and a plurality of R^{F3} may be identical or different when m7 is 2, 3, 4, 5 or 6.

In formula (Z-3), R^{ct6} to R^{ct9} are each independently halogen exclusive of iodine and fluorine, nitro, cyano, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom. The hydrocarbyl group and hydrocarbyl moiety in the hydrocarbyloxy and hydrocarbylthio groups may be saturated or unsaturated and straight, branched or cyclic. Examples thereof are as exemplified above for the hydrocarbyl group R¹ in formula (A). In the hydrocarbyl group and hydrocarbyl moiety in the hydrocarbyloxy and hydrocarbylthio groups, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety.

When m8=2, two R¹⁶ may be identical or different and two R^{ct6} may bond together to form a ring with the carbon atoms to which they are attached. When m9=2, two R^{ct7} may be identical or different and two R^{ct7} may bond together to form a ring with the carbon atoms to which they are attached. When m10=2, two R^{ct8} may be identical or different and two R^{ct8} may bond together to form a ring with the carbon atoms to which they are attached. When m13=2, two R^{ct9} may be identical or different and two R^{ct9} may bond together to form a ring with the carbon atoms to which they are attached. Examples of the ring thus formed include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

The aromatic rings directly bonded to S⁺ in the sulfonium cation having formula (Z-3) may bond together to form a ring with S⁺. Exemplary structures of the ring are shown below.

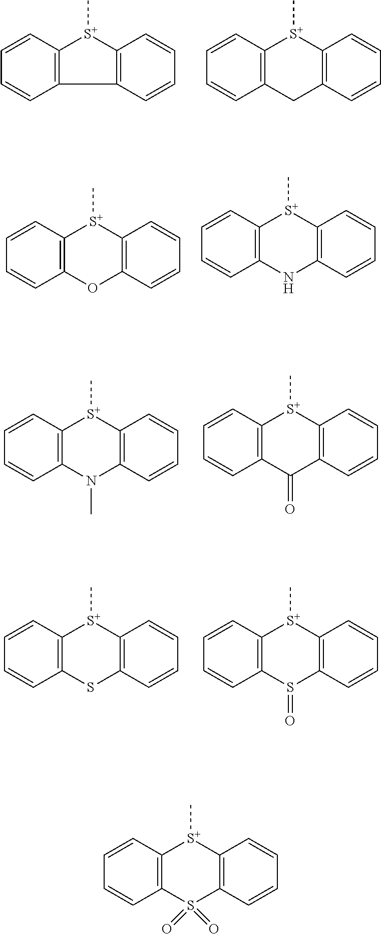

In formula (Z-3), L^{D} and L^{E} are each independently a single bond, ether bond, ester bond, sulfonate ester bond, amide bond, sulfonamide bond, carbonate bond or carbamate bond. L^{D} is preferably a single bond, ether bond, ester bond or sulfonate ester bond, more preferably an ester bond or sulfonate ester bond. L^{E} is preferably a single bond, ether bond or ester bond, more preferably a single bond.

In formula (Z-3), X^{L3} is a single bond or a C₁-C₄₀ hydrocarbylene group which may contain a heteroatom. The C₁-C₄₀ hydrocarbylene group may be straight, branched or cyclic. Suitable hydrocarbylene groups include alkanediyl, cyclic saturated hydrocarbylene and arylene groups. Suitable heteroatoms include oxygen, nitrogen and sulfur atoms.

Examples of the optionally heteroatom-containing C₁-C₄₀ hydrocarbylene group X^{L3} are as exemplified above for the optionally heteroatom-containing C₁-C₄₀ hydrocarbylene group X^{L2} in formula (A), but not limited thereto. Of X^{L}-0 to X^{L}-58, X^{L3} is preferably selected from X^{L}-0 to X^{L}-22, X^{L}-29 to X^{L}-34, and X^{L}-47 to X^{L}-58.

Of the sulfonium cations having formula (Z-3), those having the formula (Z-3-1) are preferred.

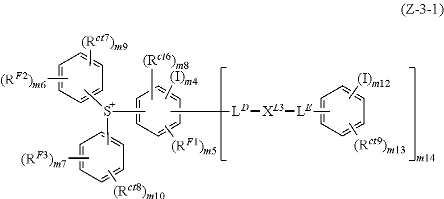

Herein m4 to m10, m12 to m14, R^{F1} to R^{F3}, R^{ct6} to R^{ct9}, L^{D}, L^{E}, and X^{L3} are as defined above.

Of the sulfonium cations having formula (Z-3-1), those having the formula (Z-3-2) are more preferred.

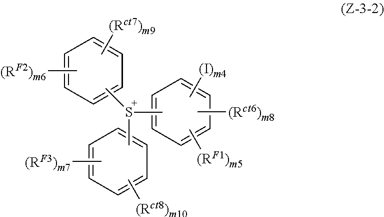

Herein m4 to m10, R^{F1} to R^{F3}, and R^{ct6} to R^{ct8} are as defined above.

Examples of the sulfonium cation having formula (Z-3) are shown below, but not limited thereto.

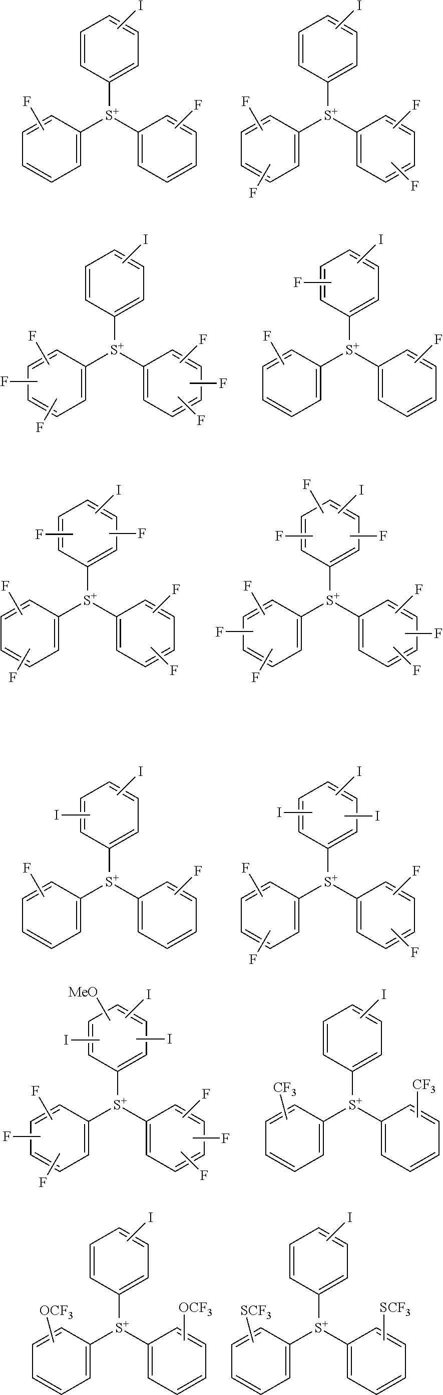

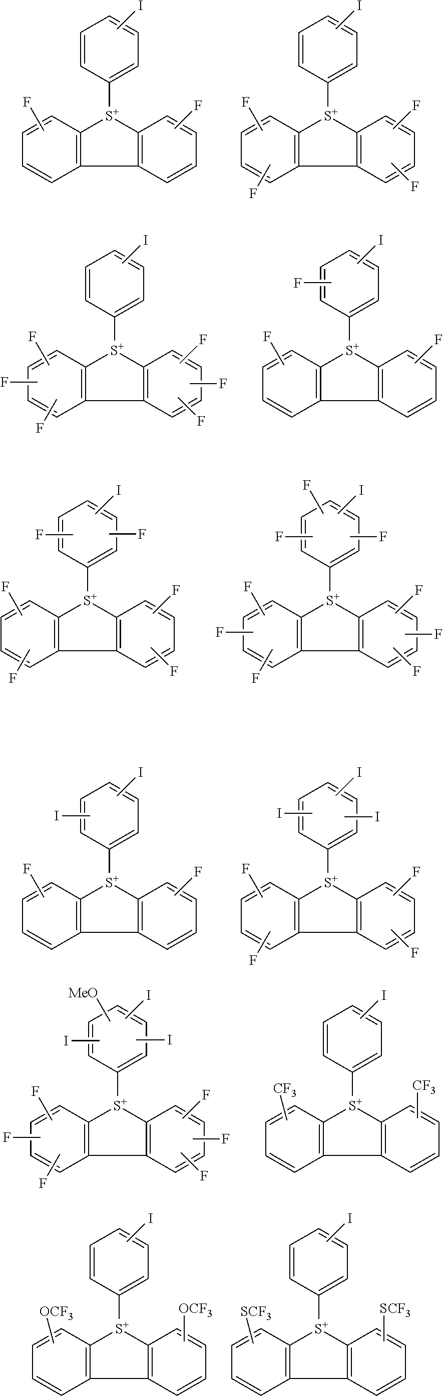

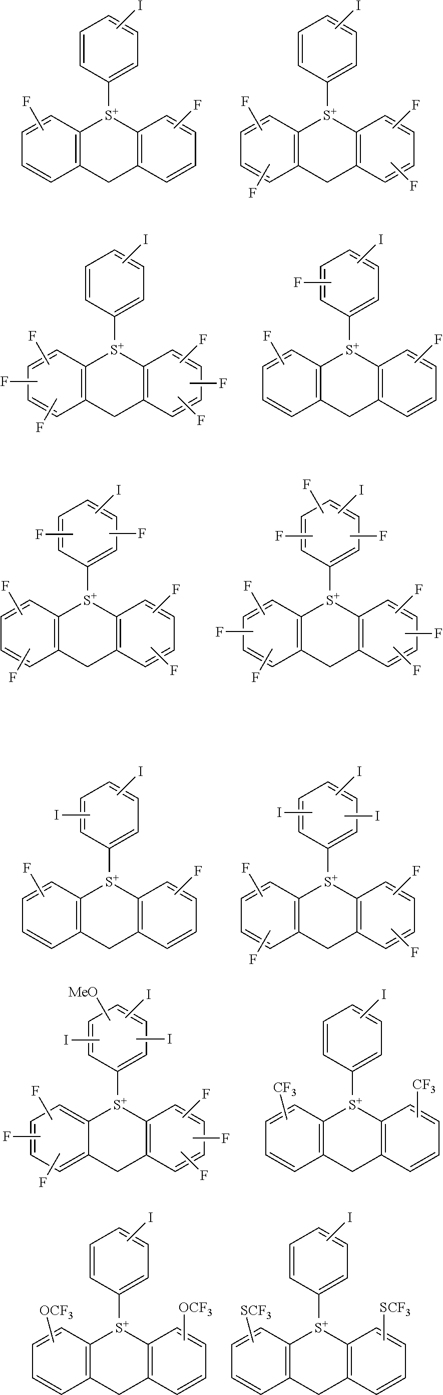

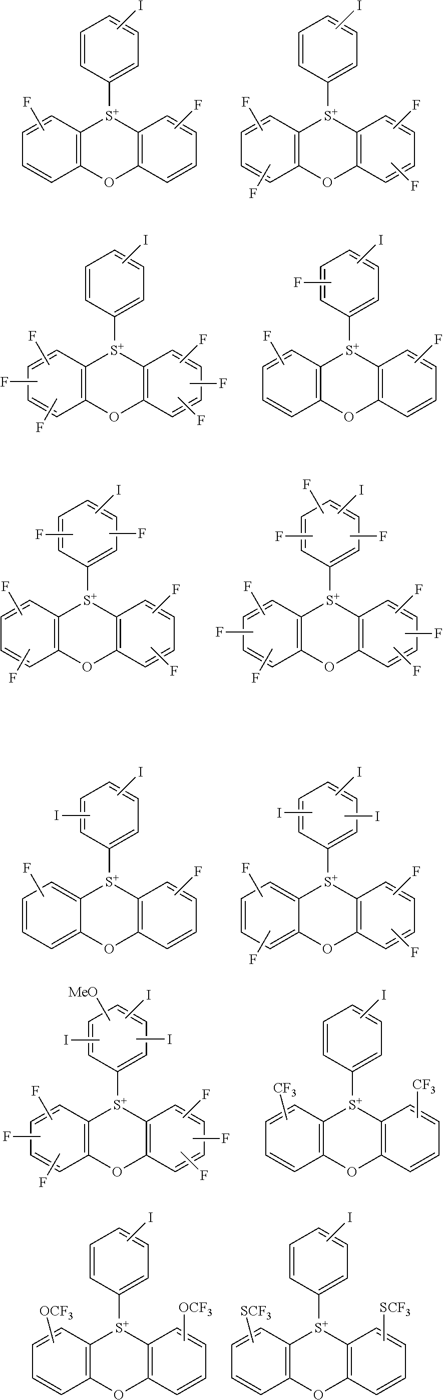

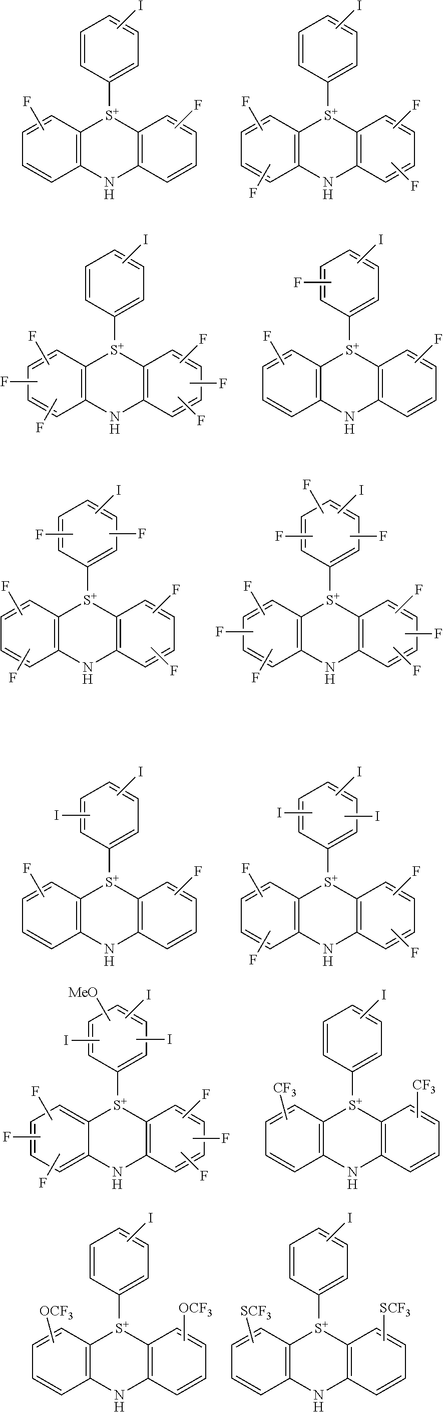

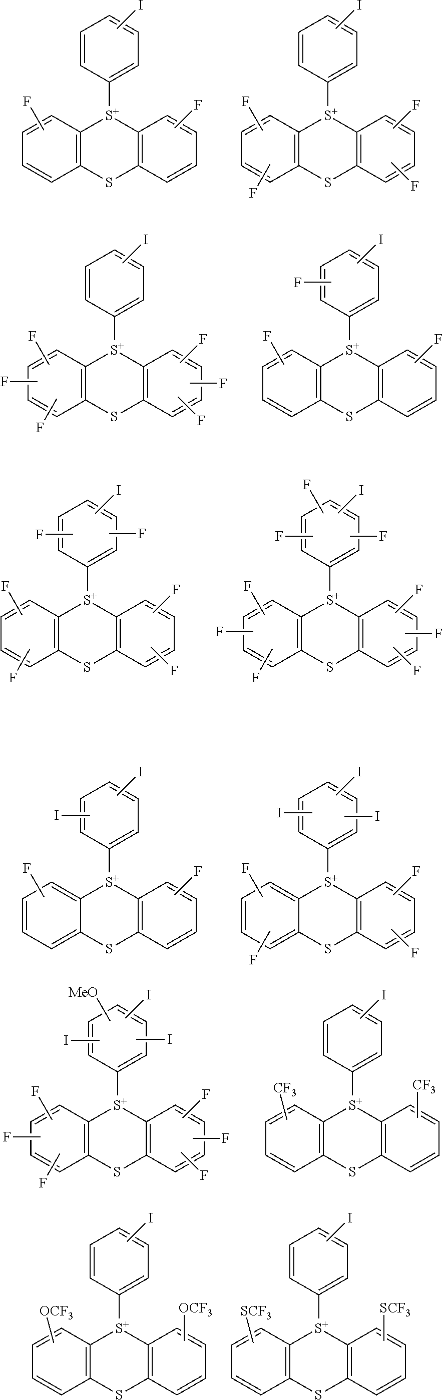

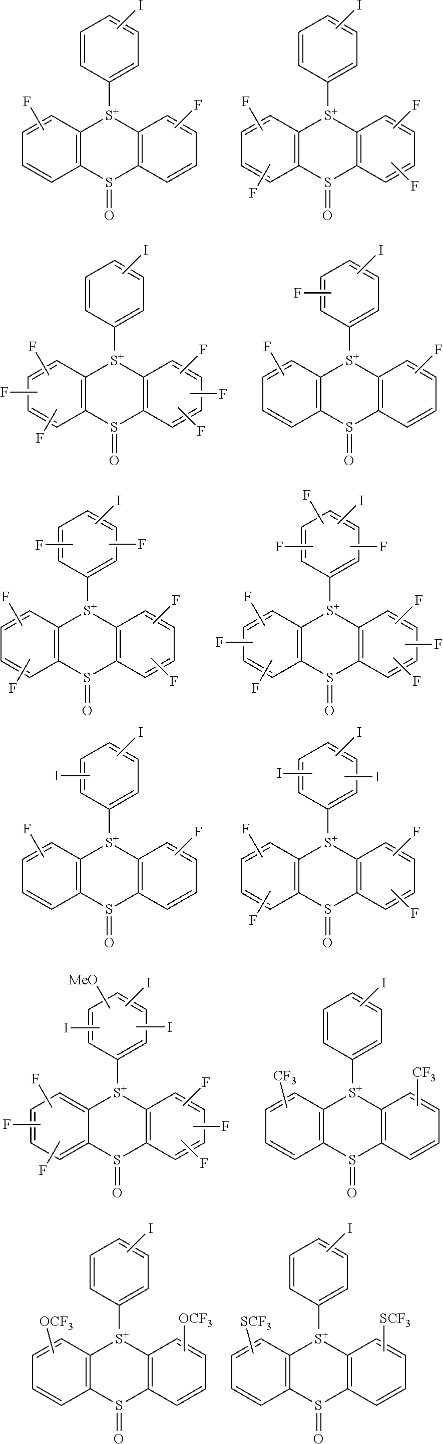

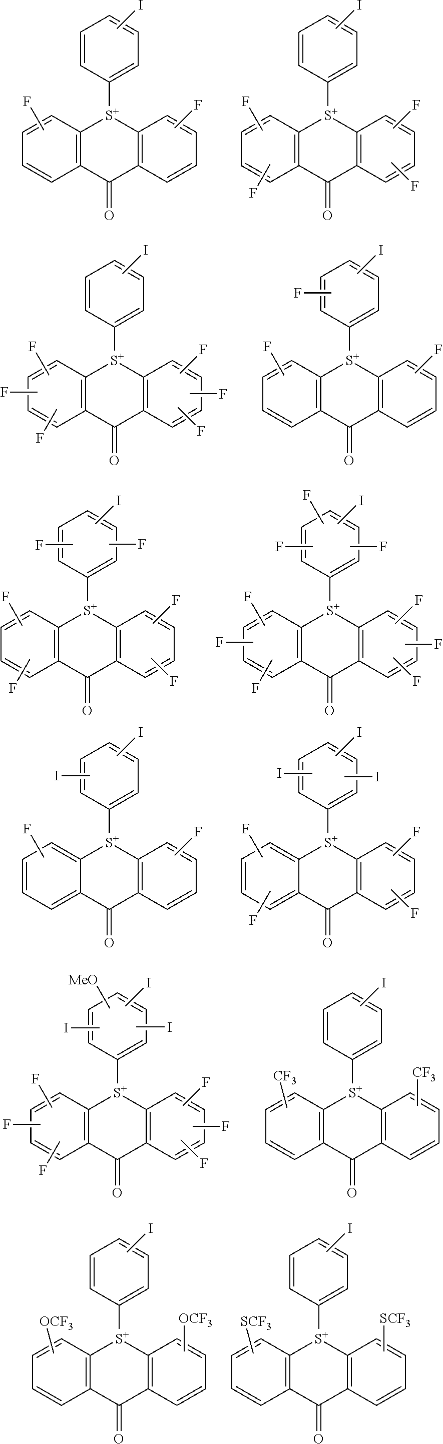

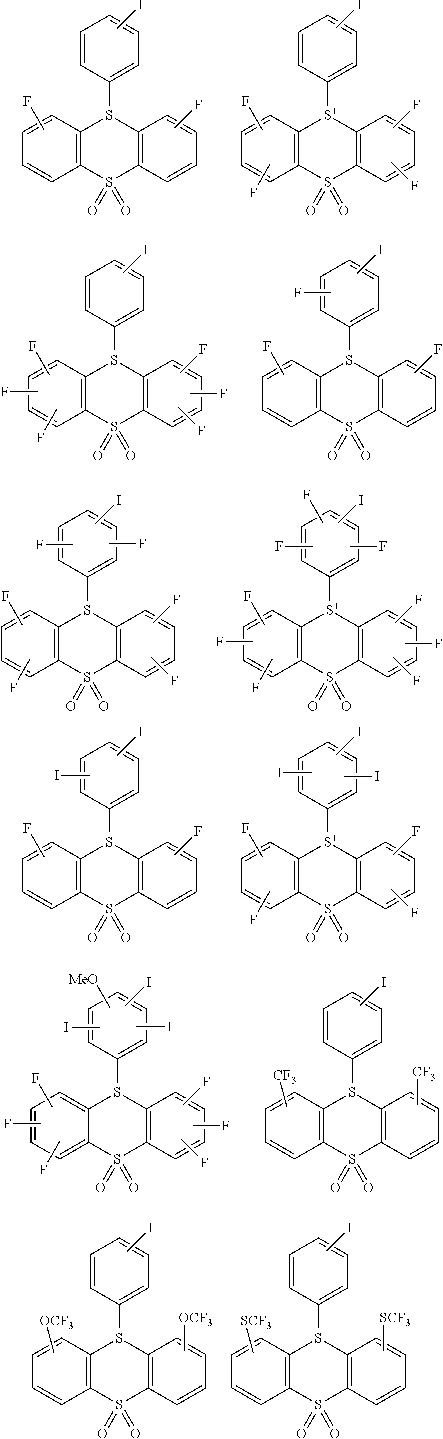

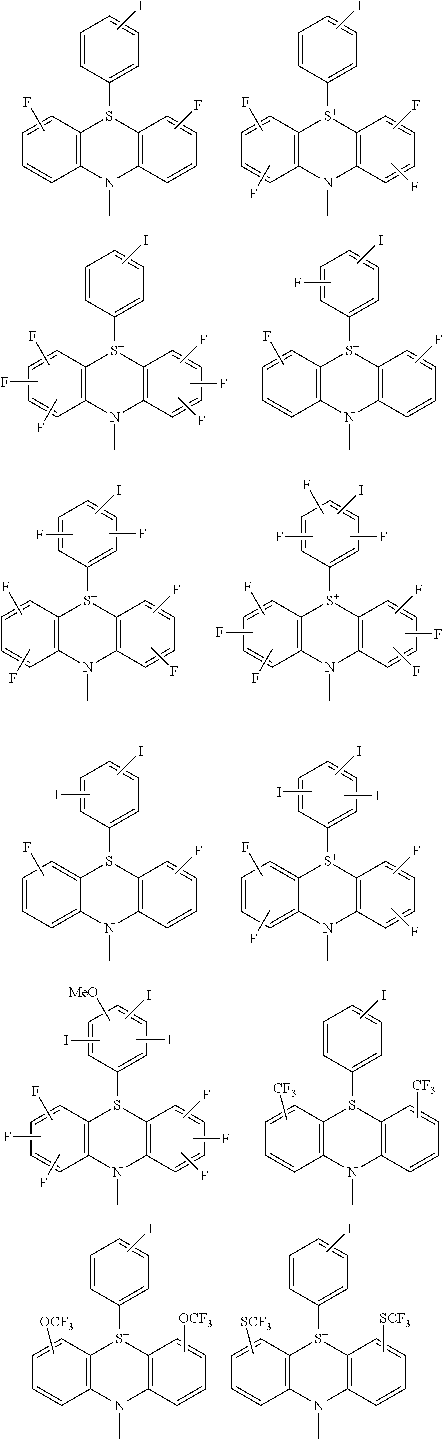

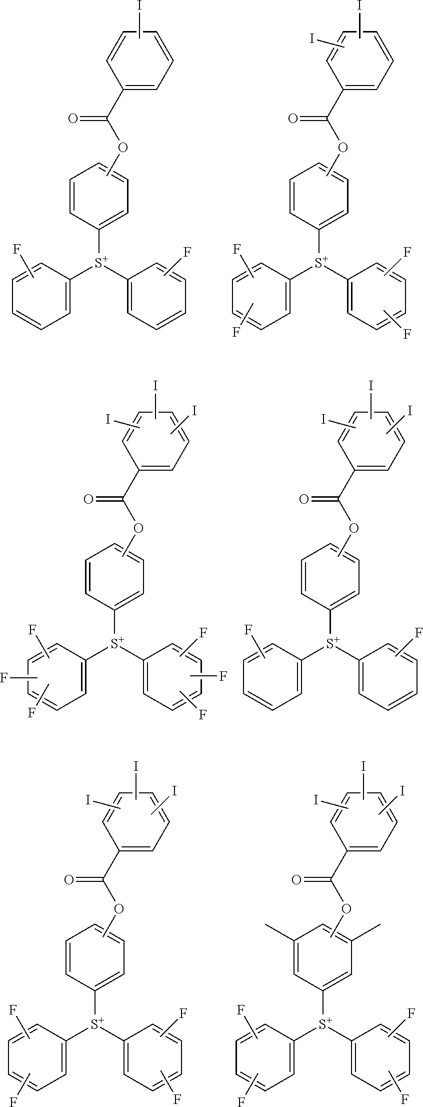

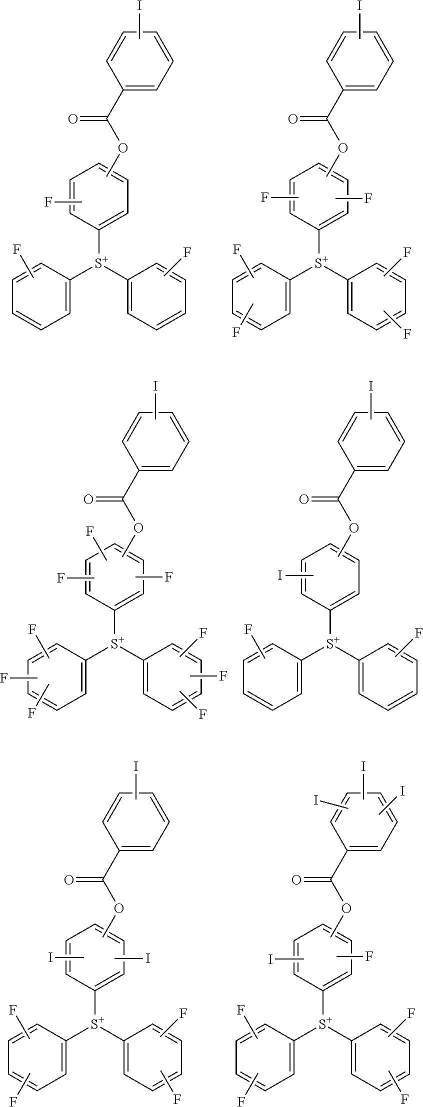

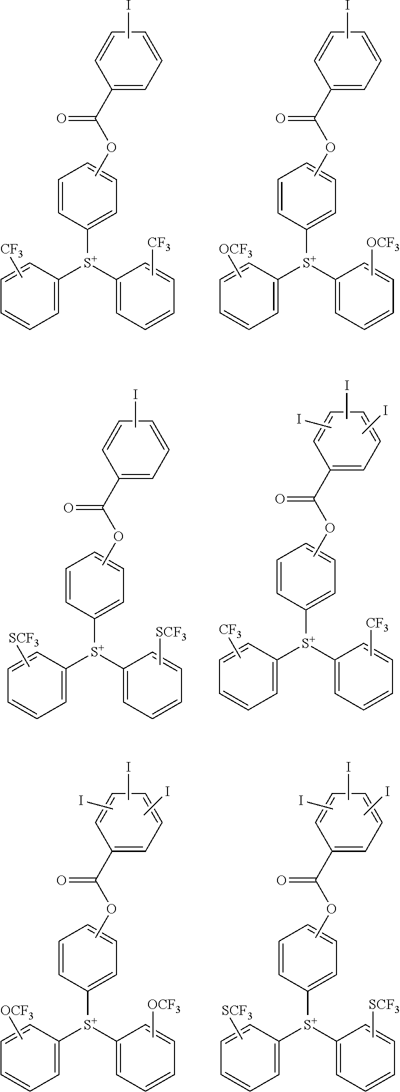

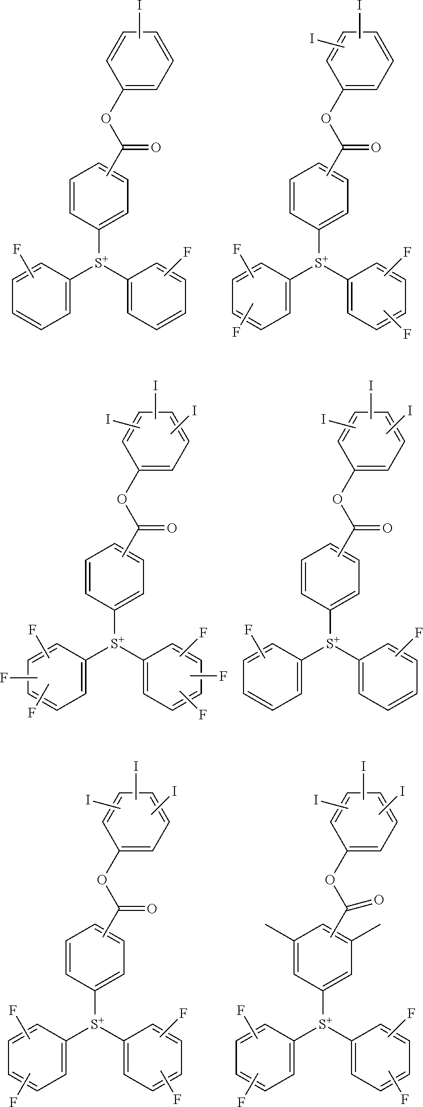

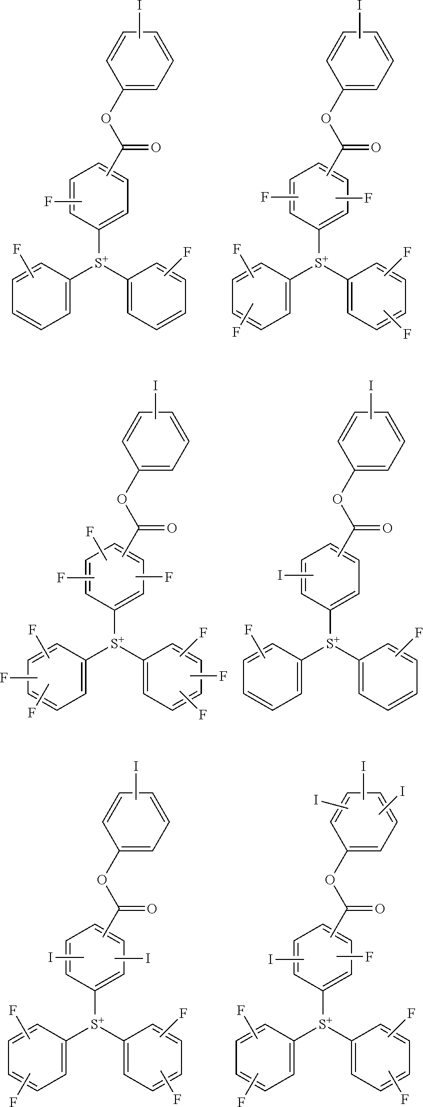

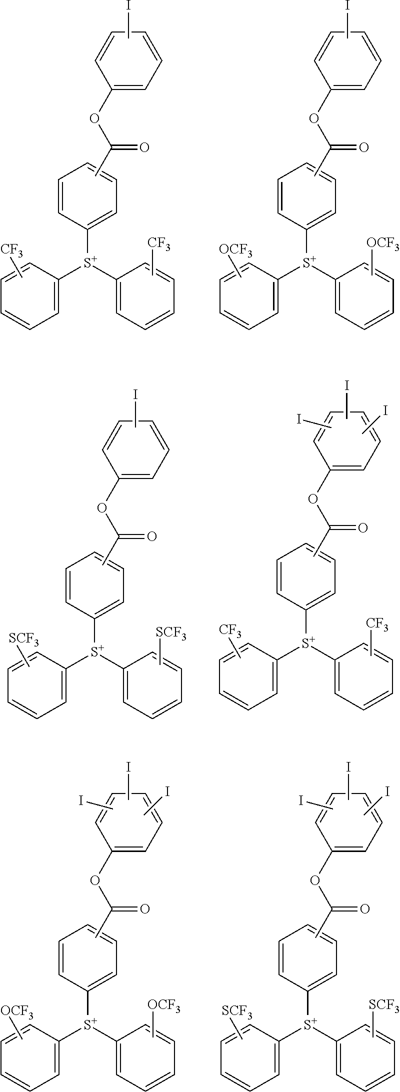

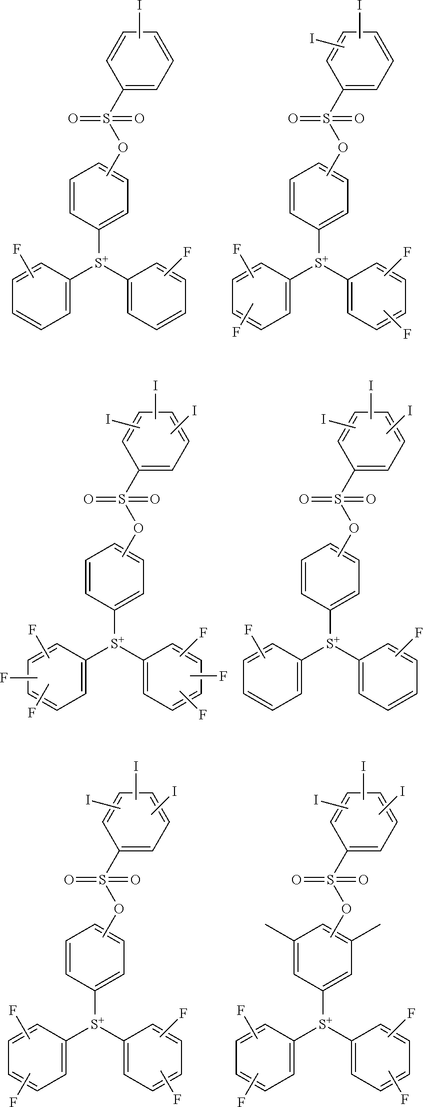

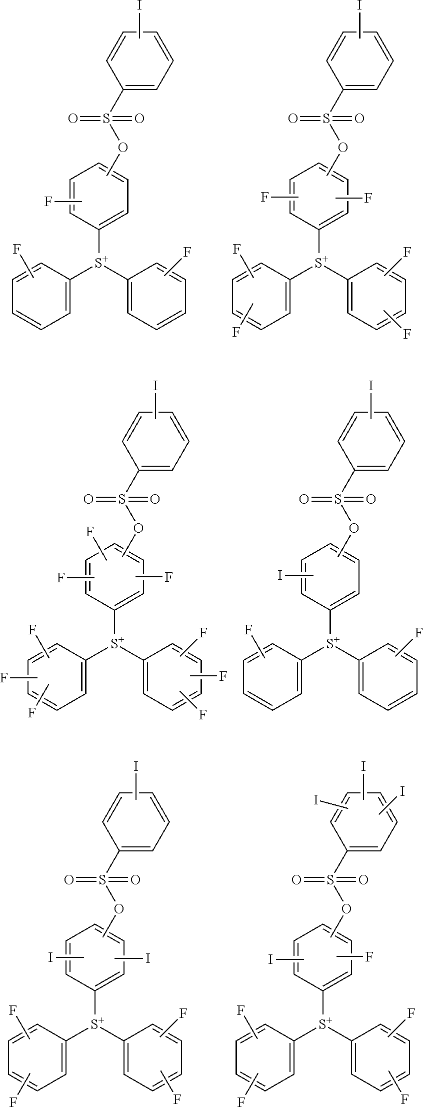

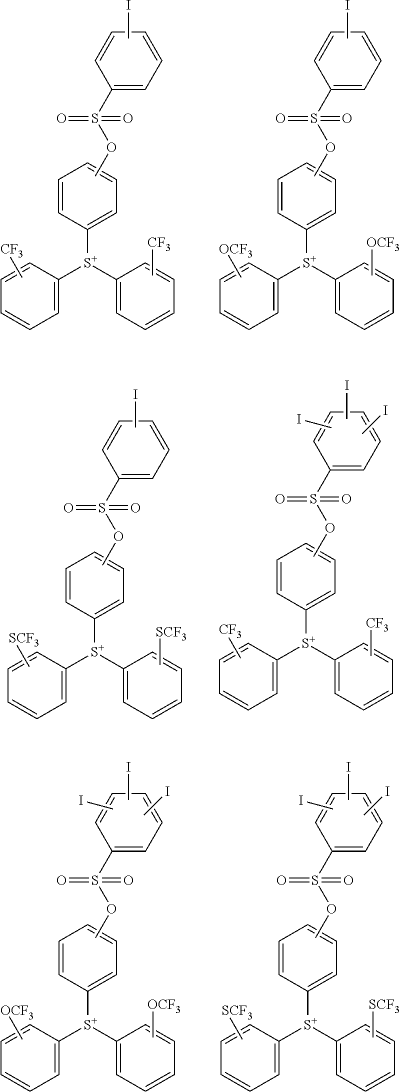

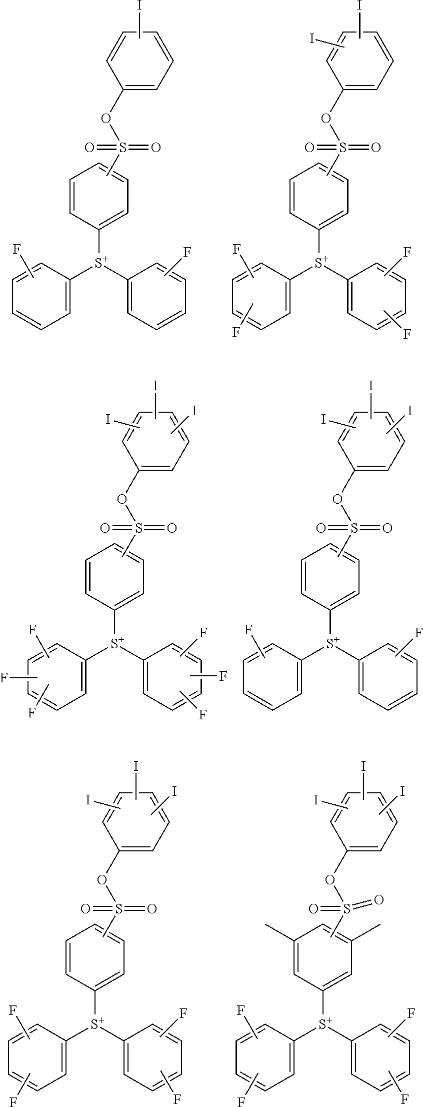

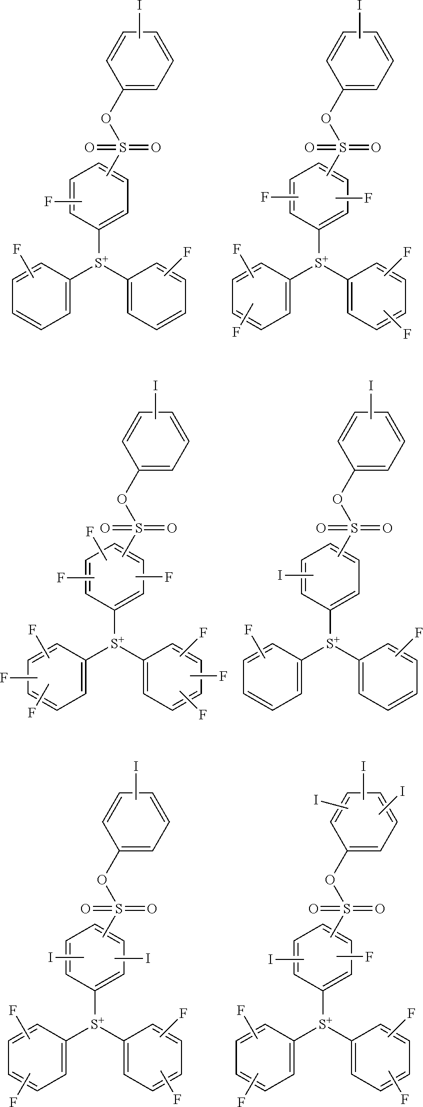

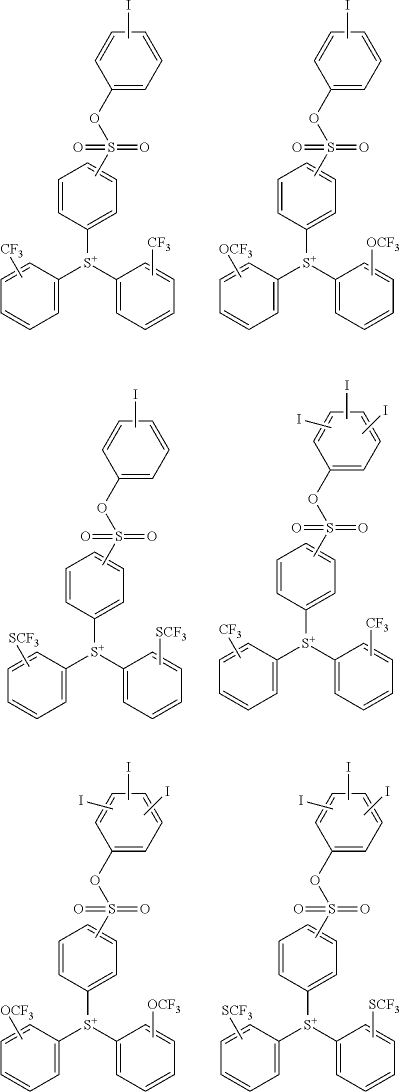

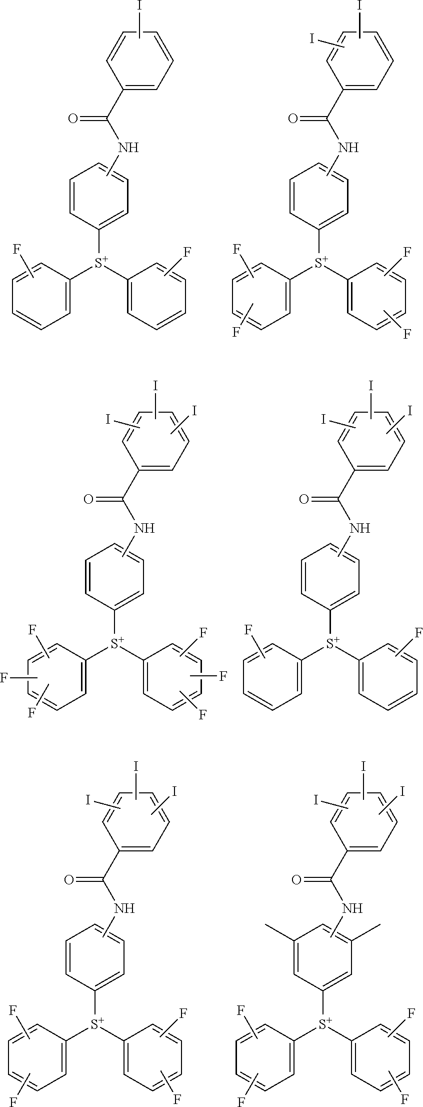

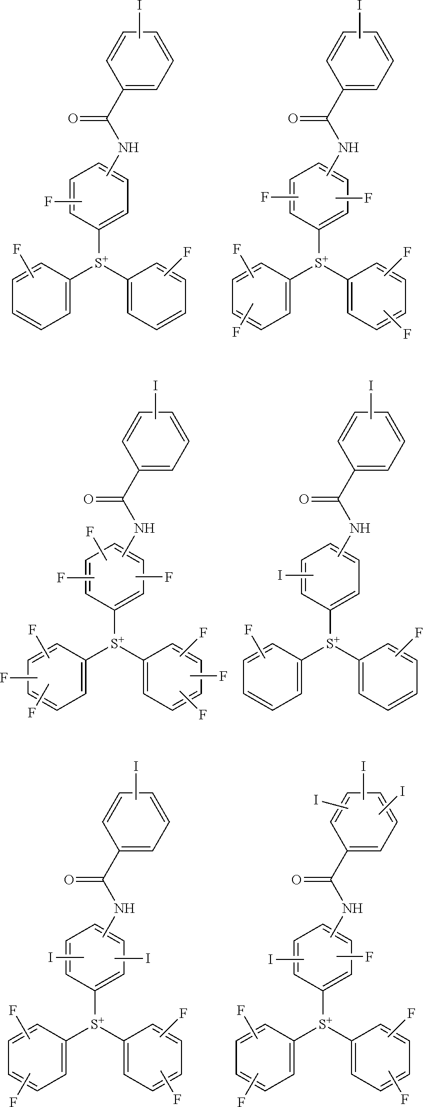

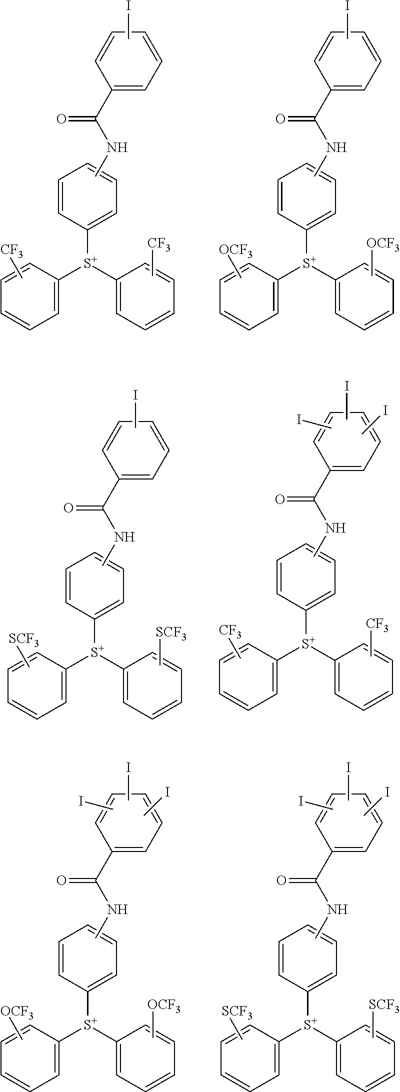

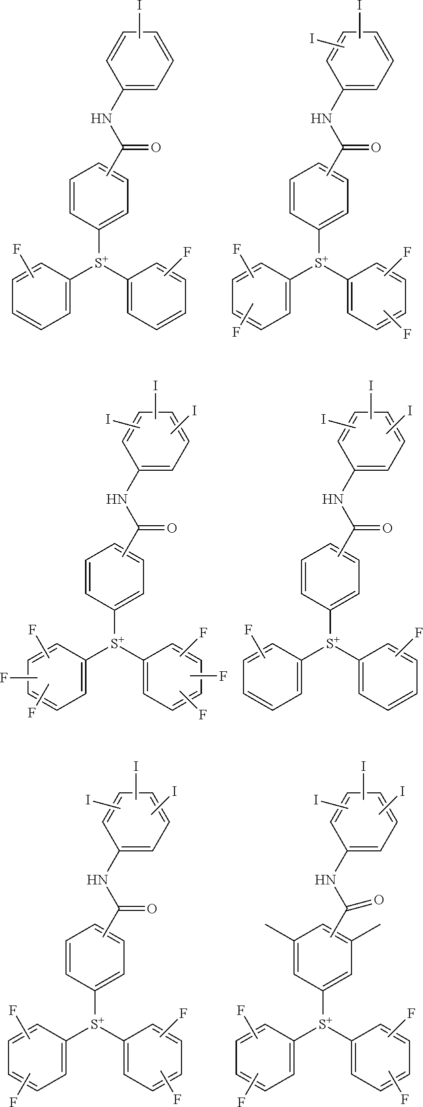

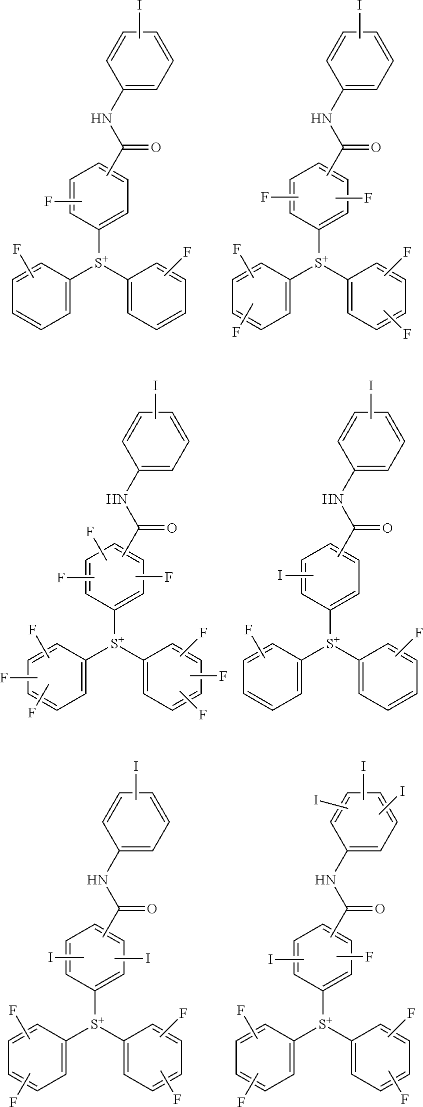

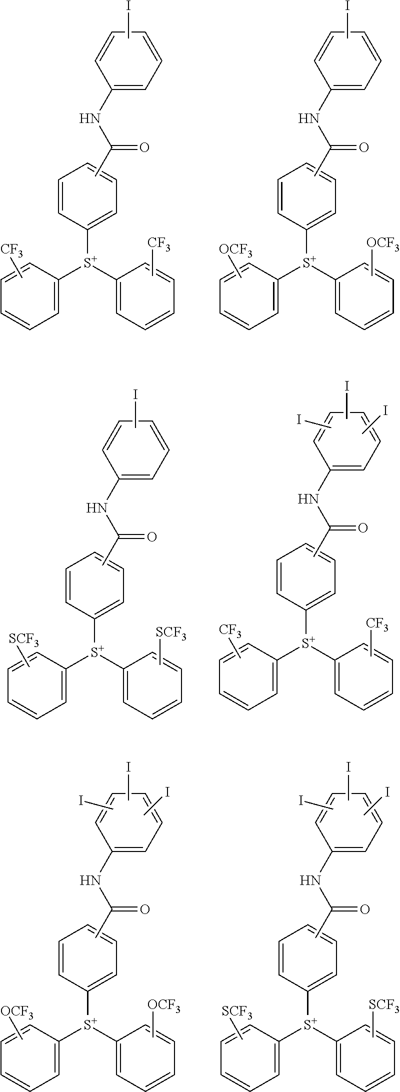

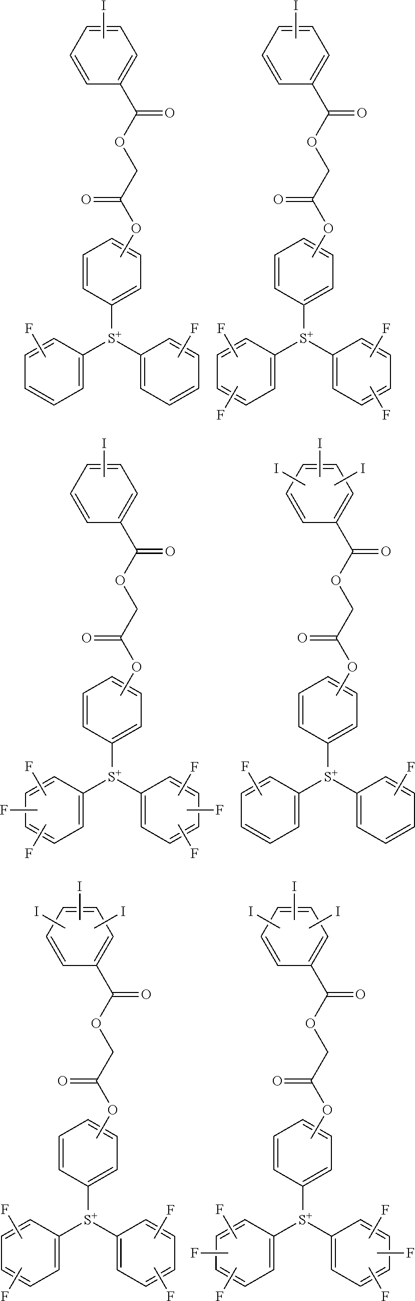

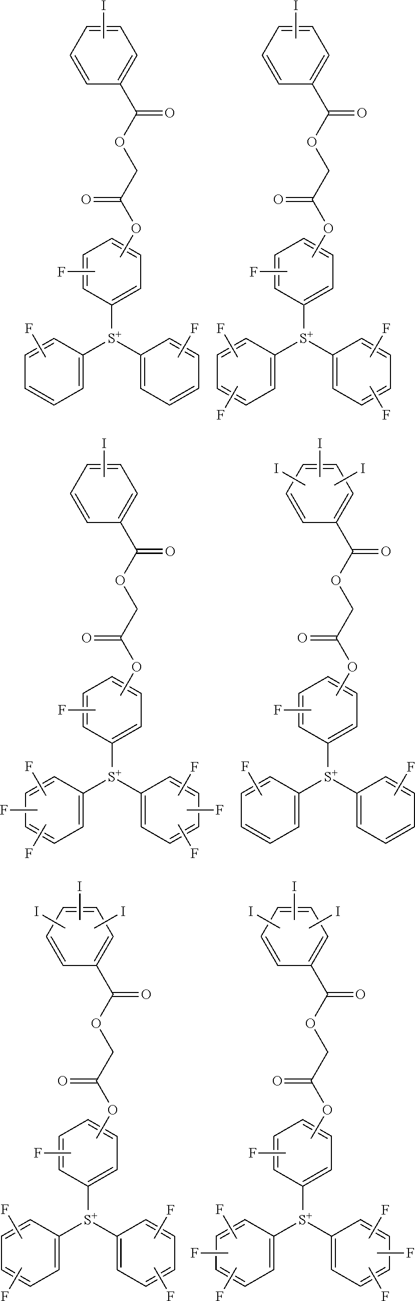

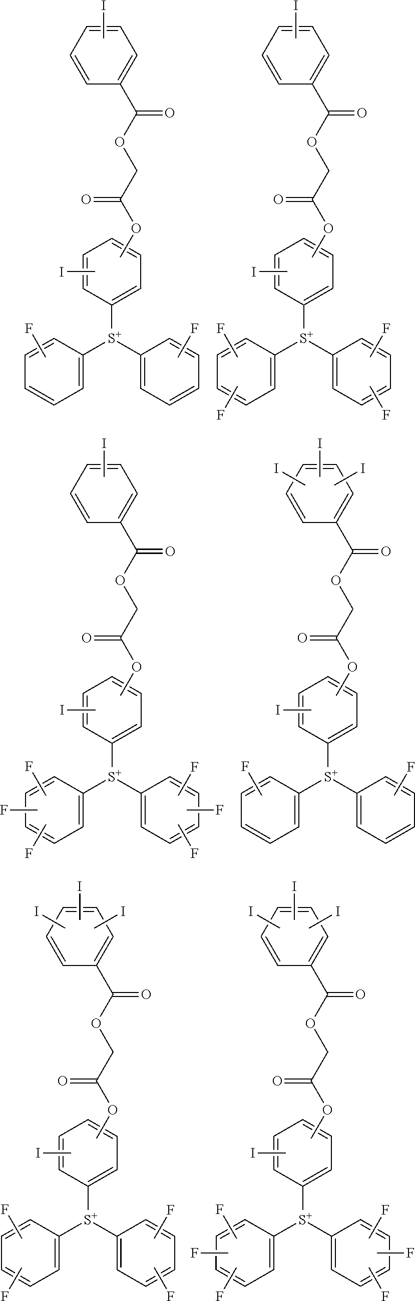

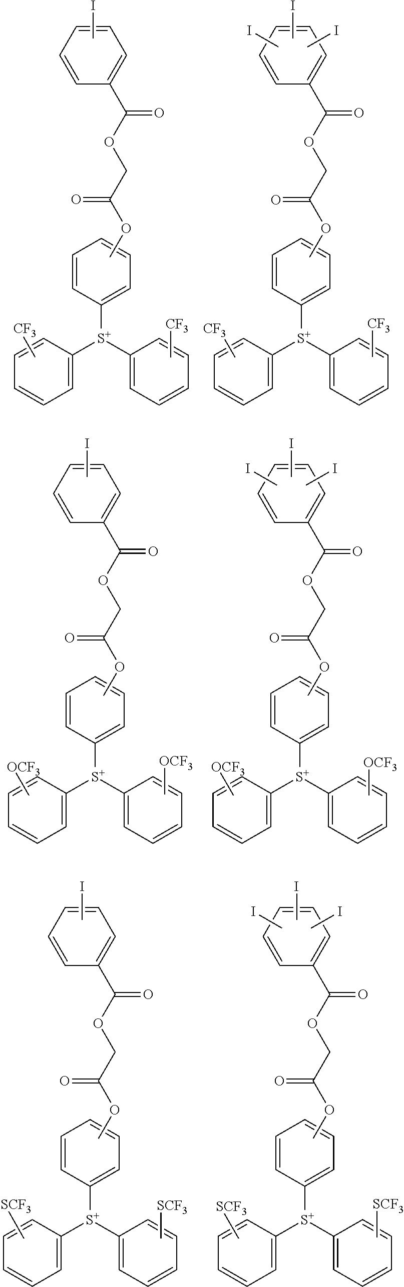

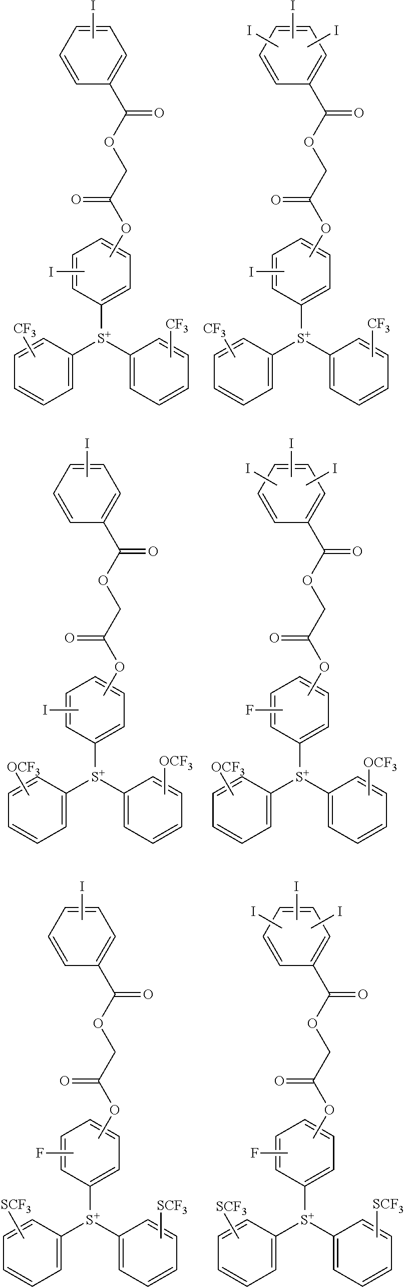

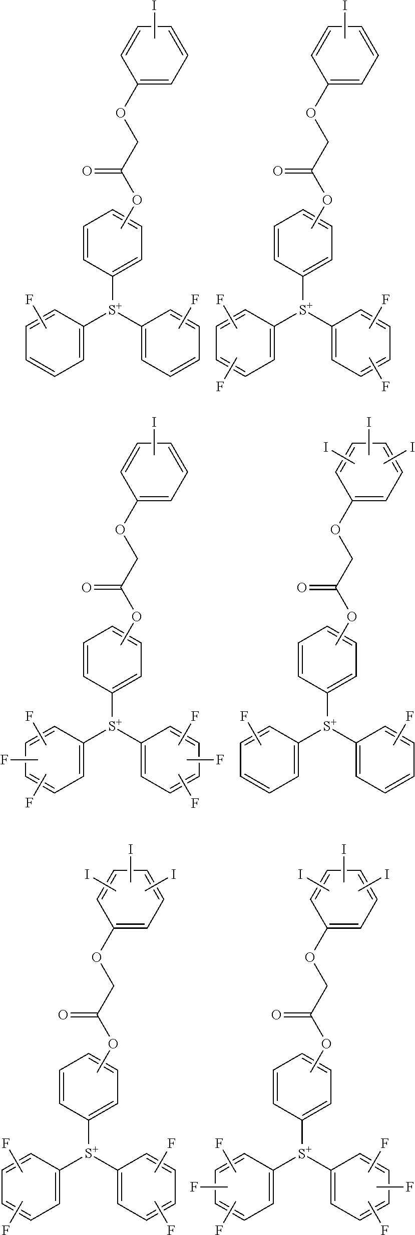

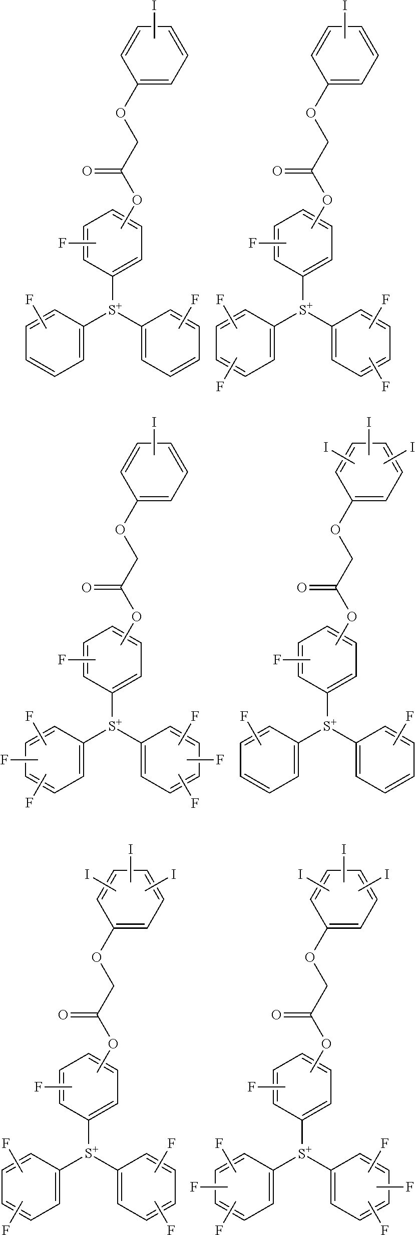

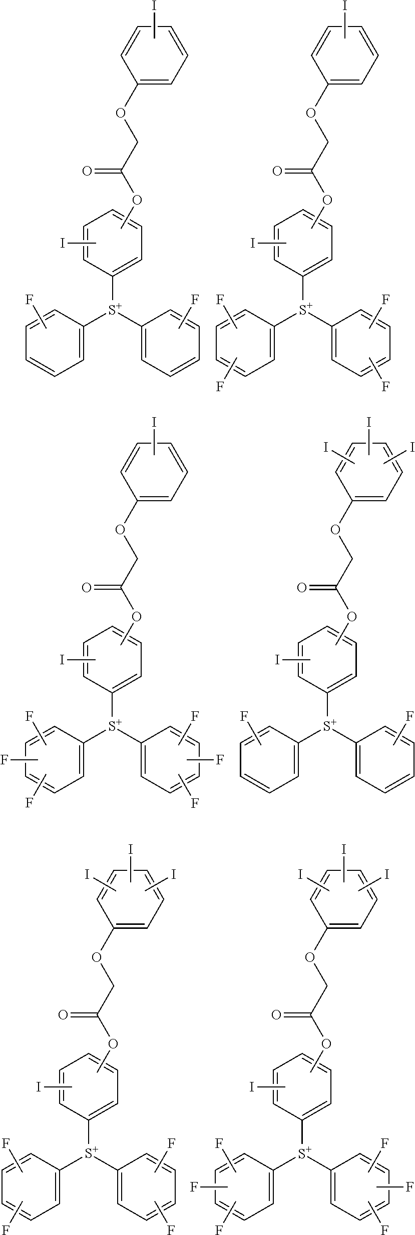

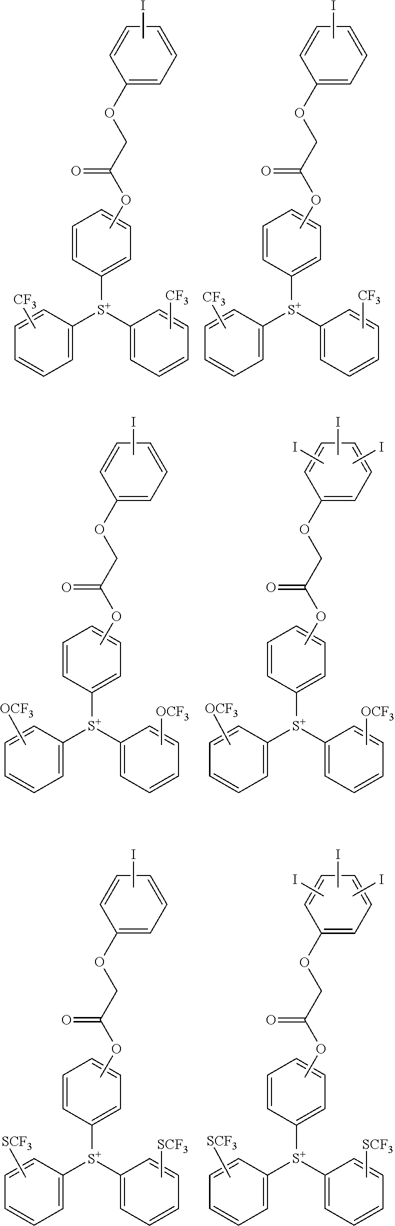

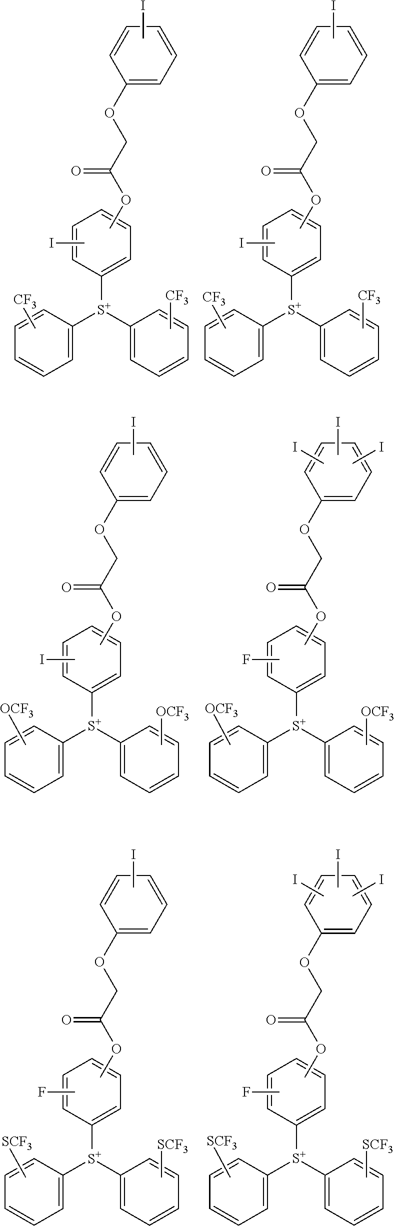

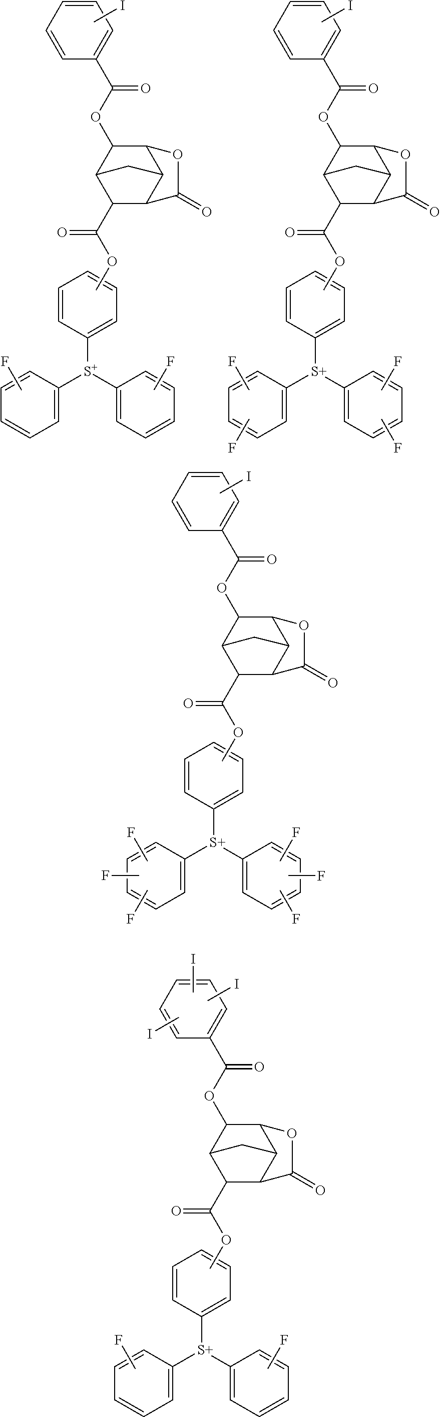

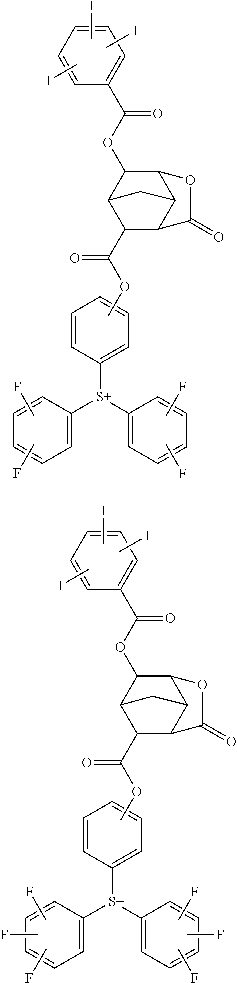

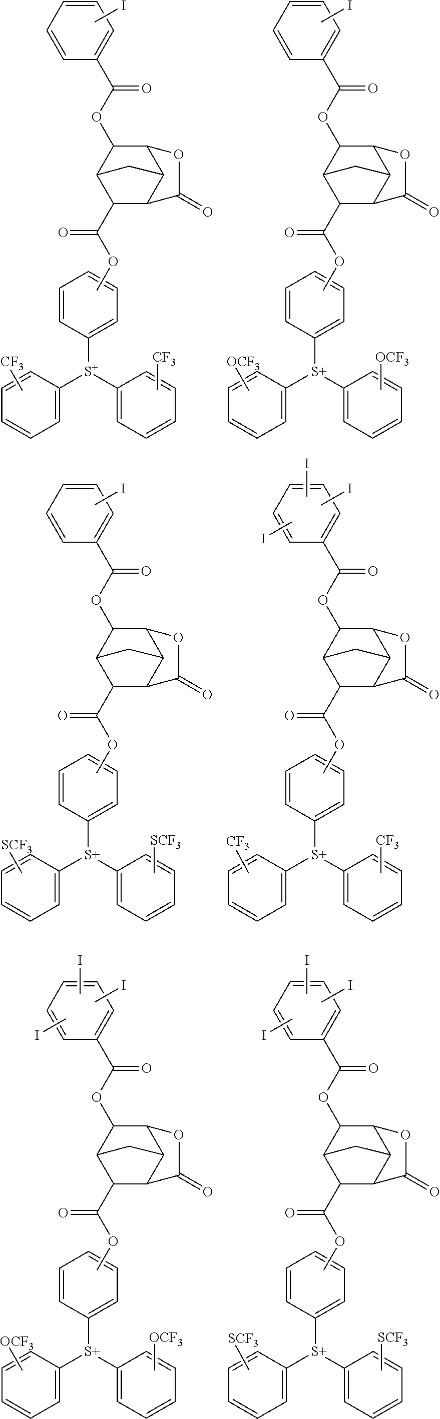

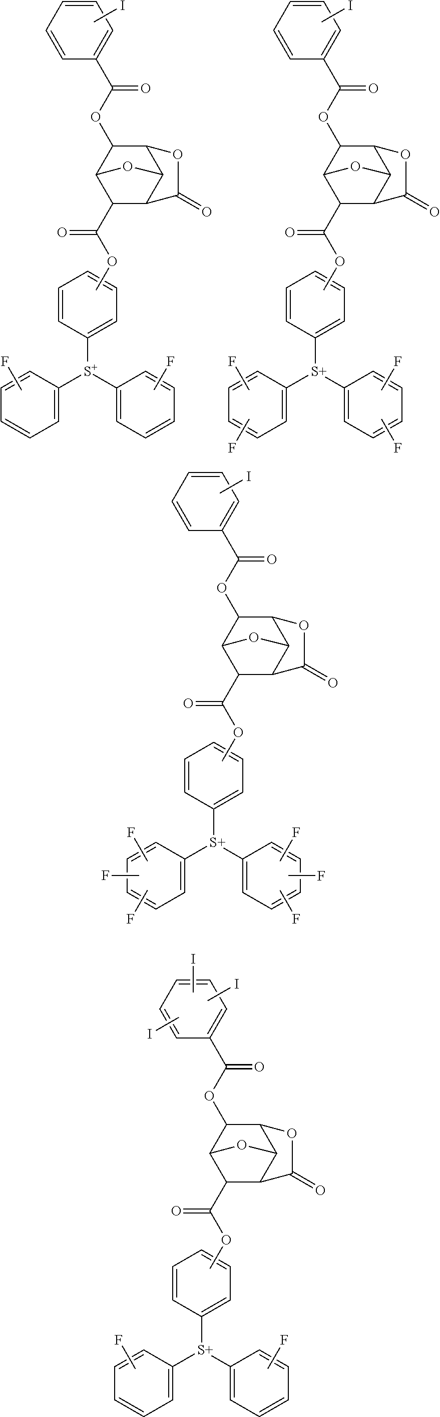

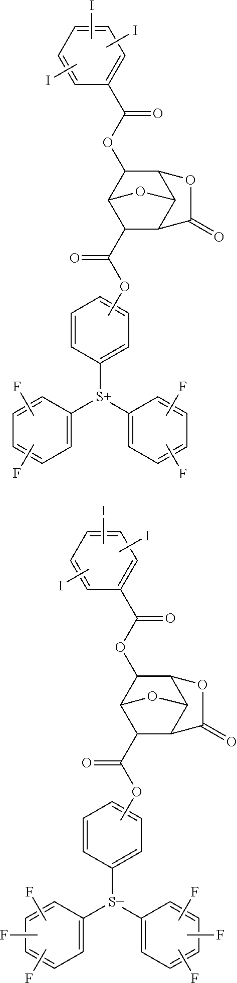

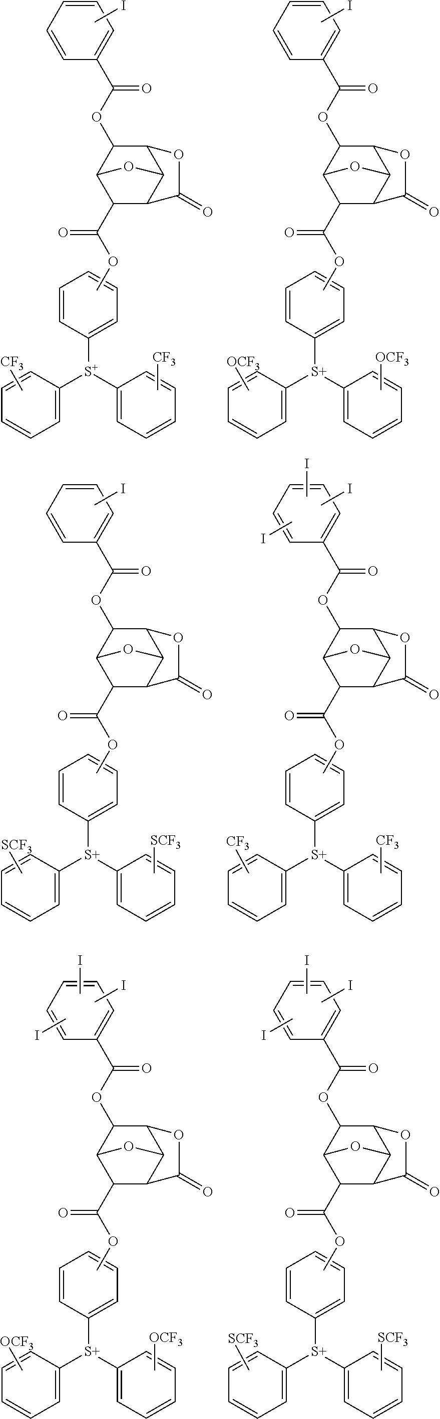

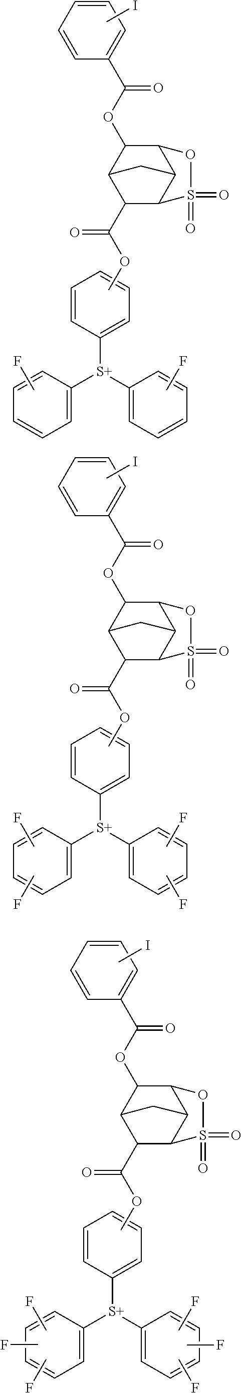

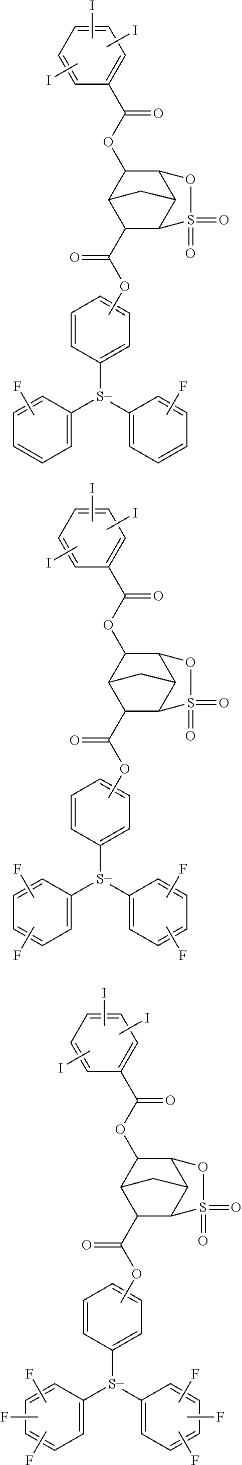

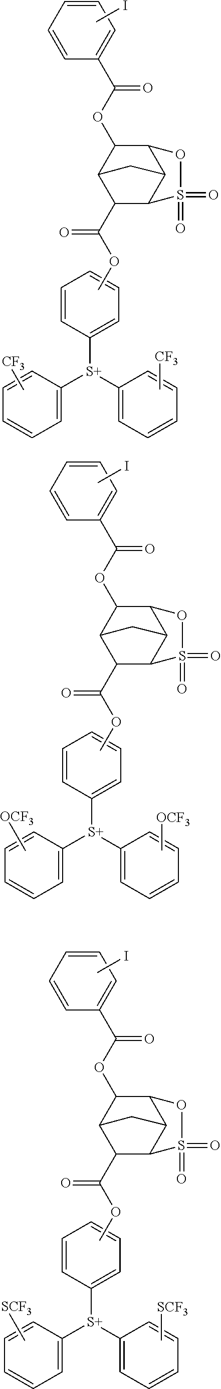

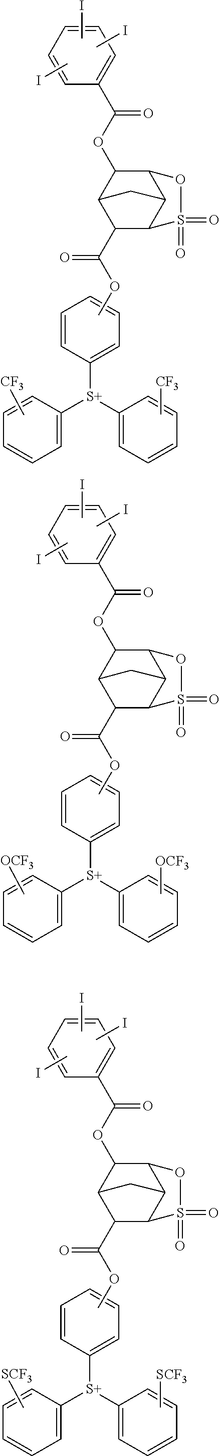

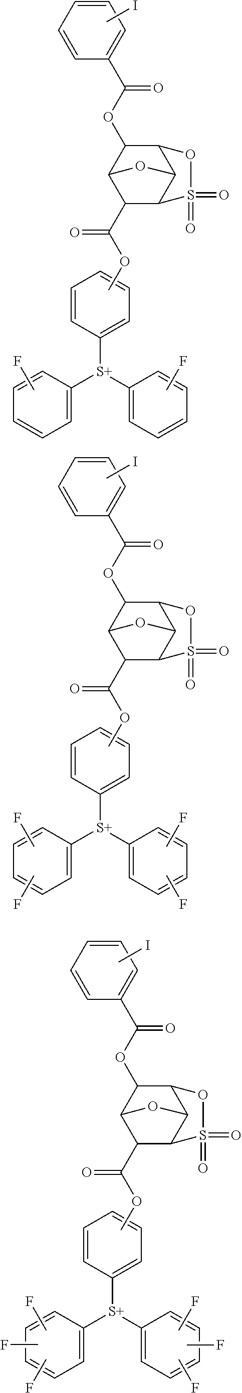

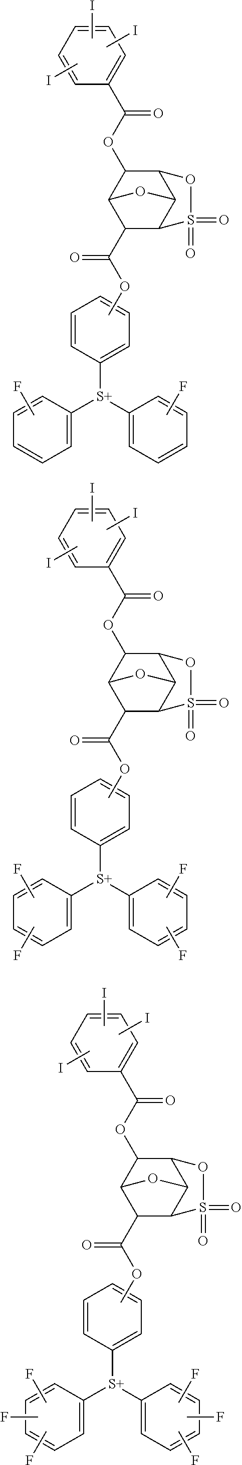

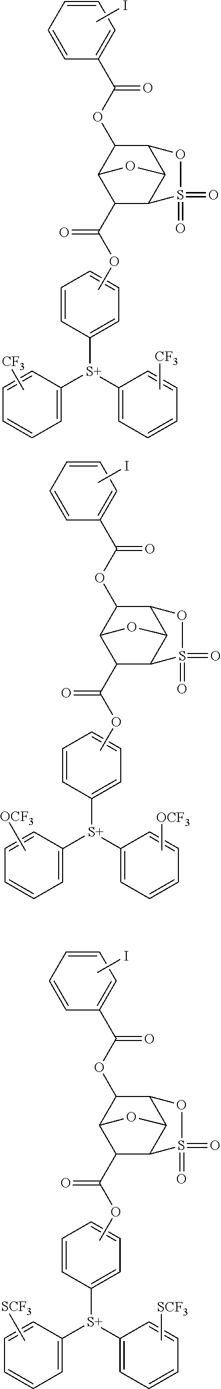

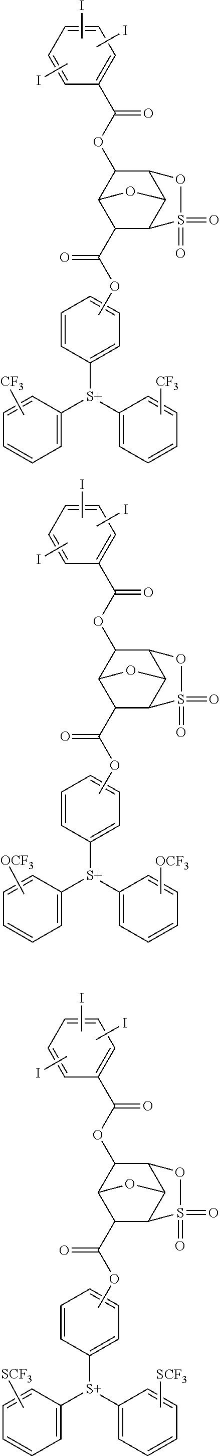

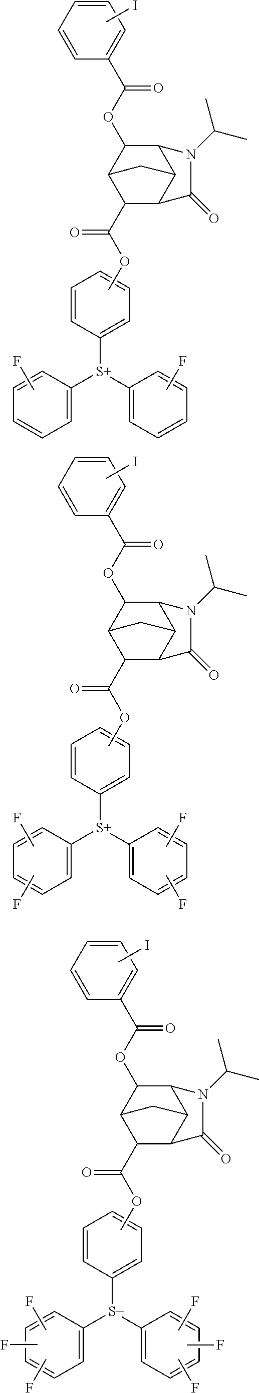

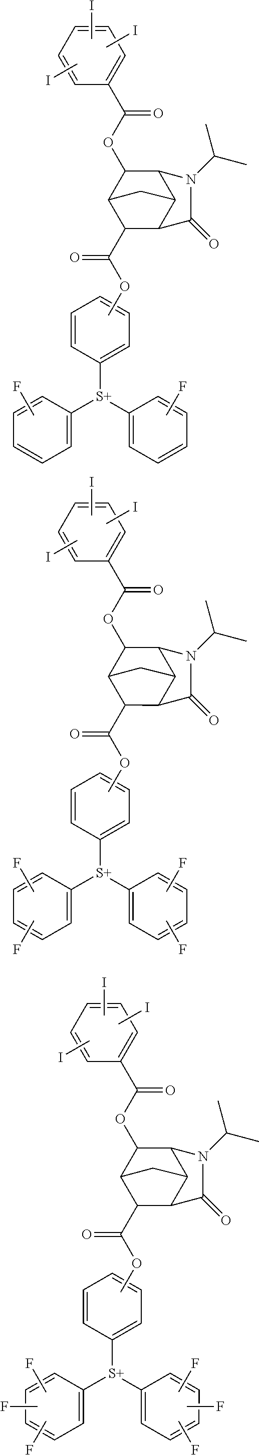

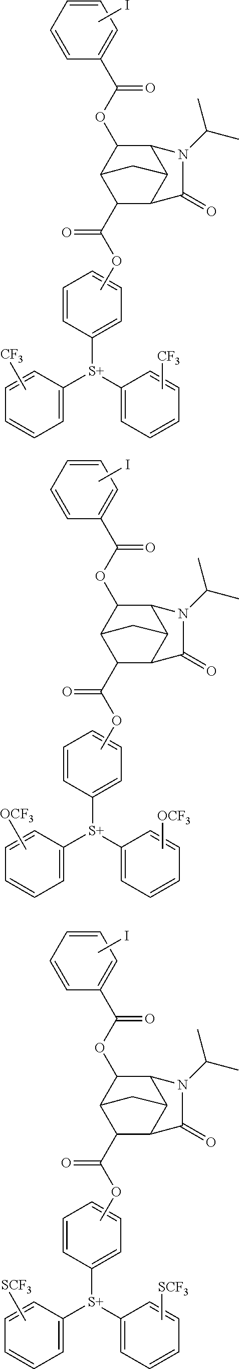

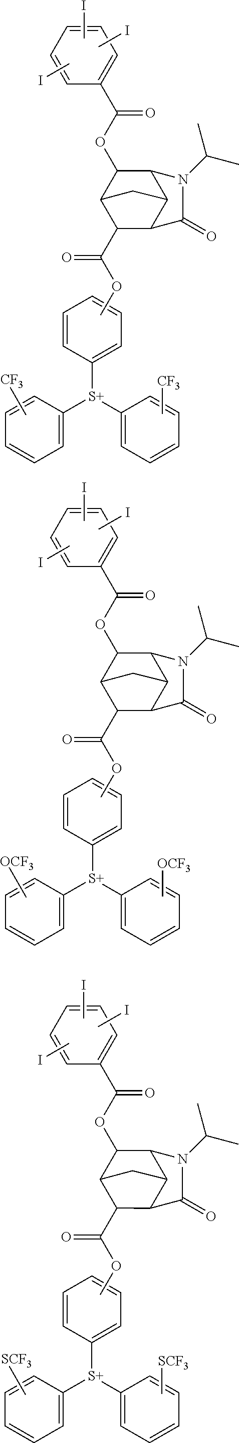

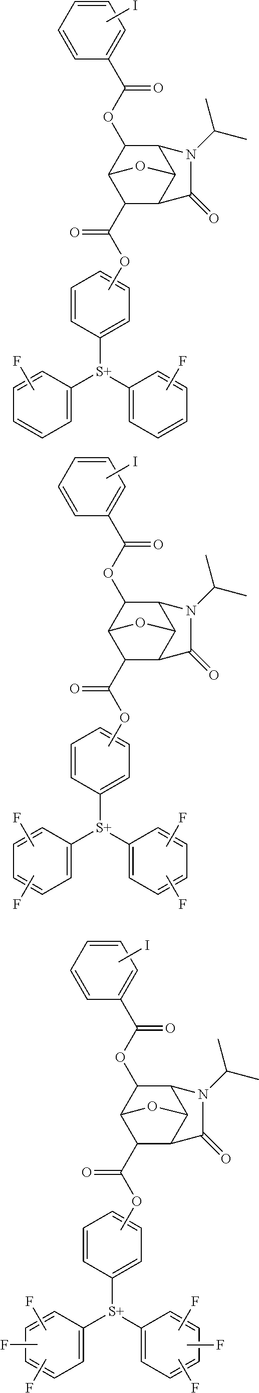

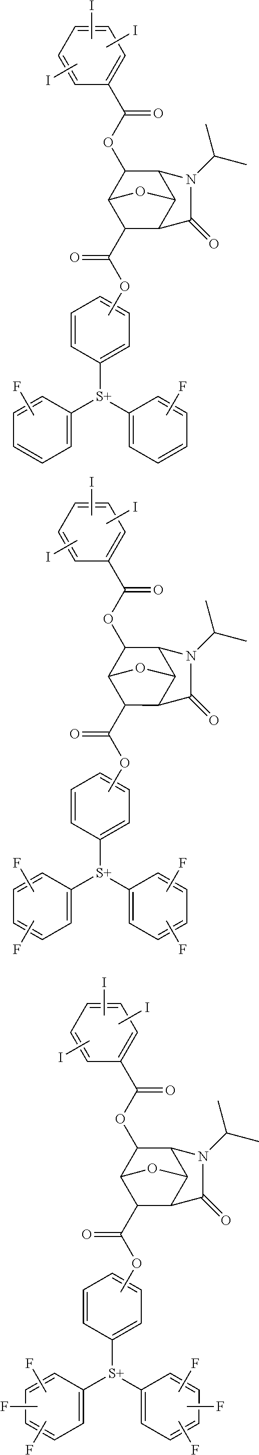

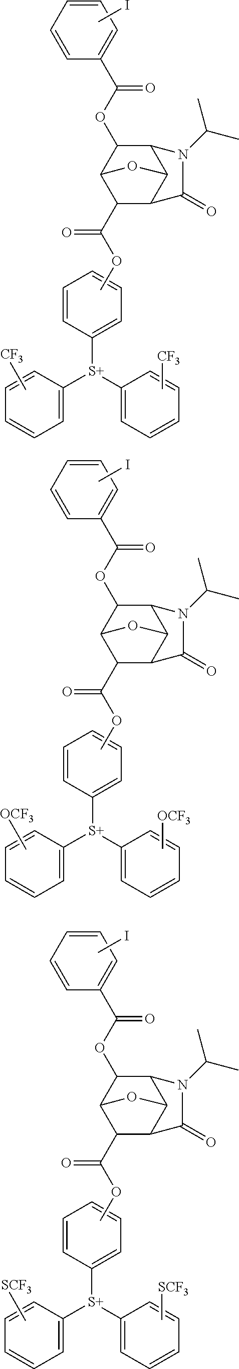

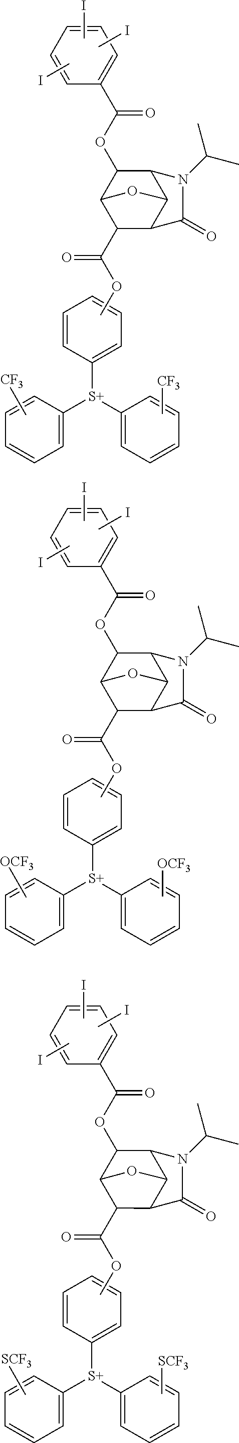

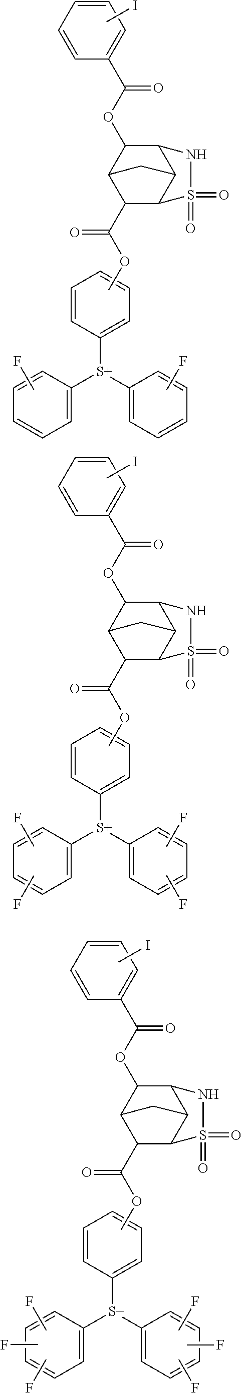

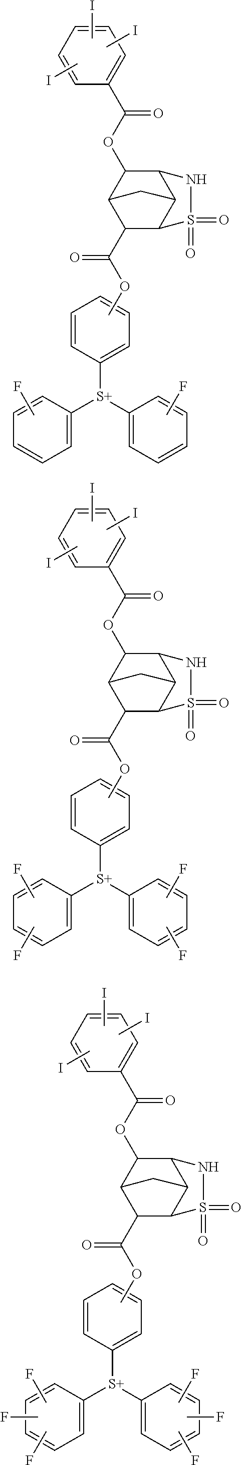

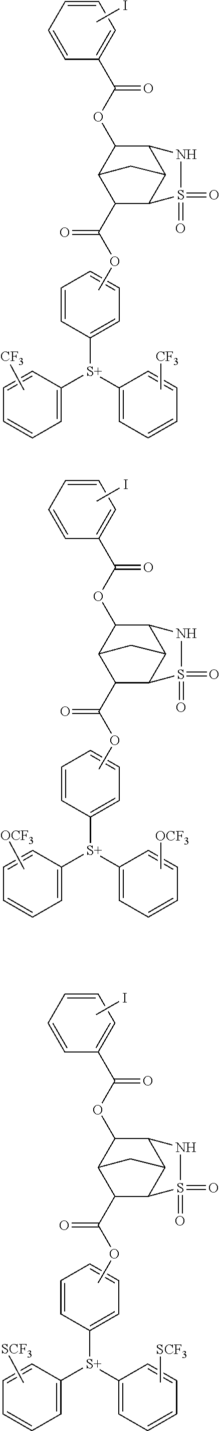

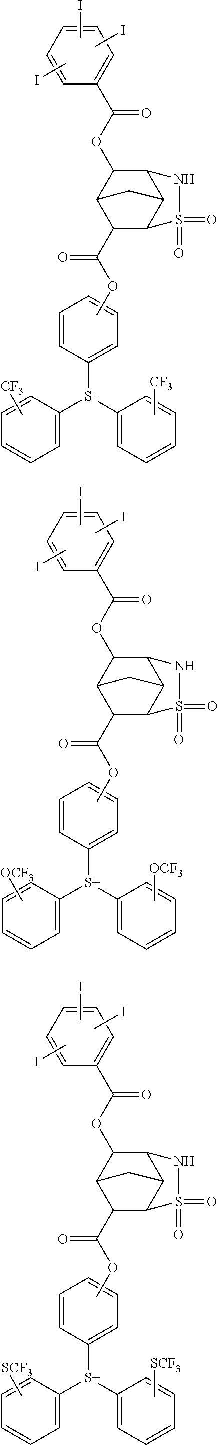

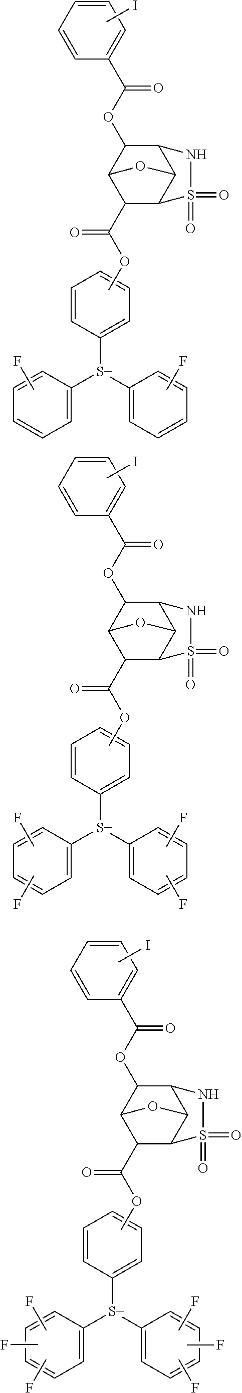

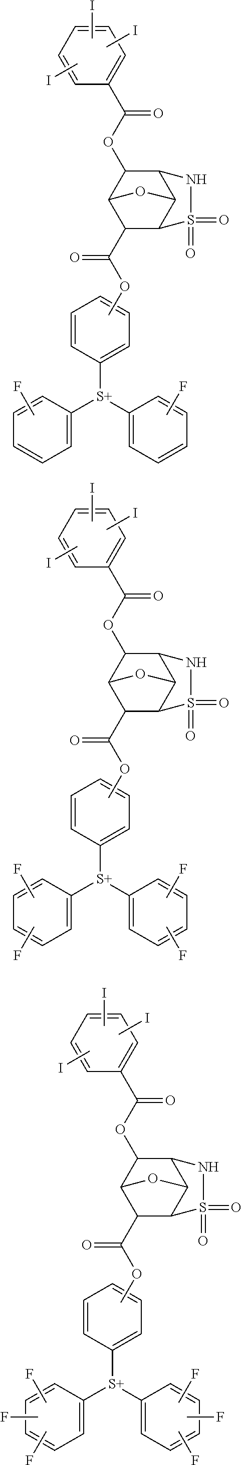

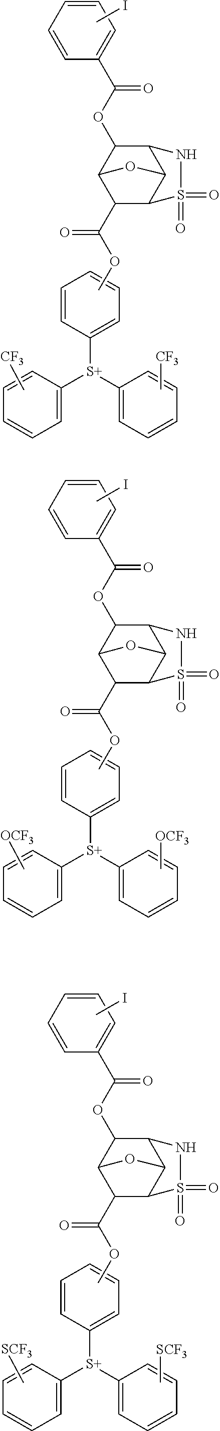

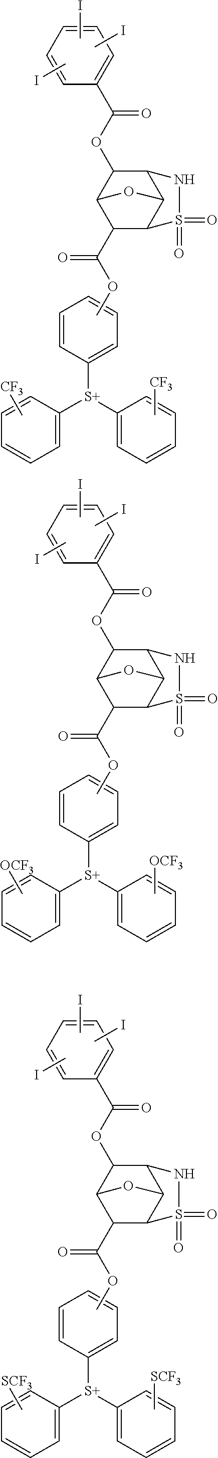

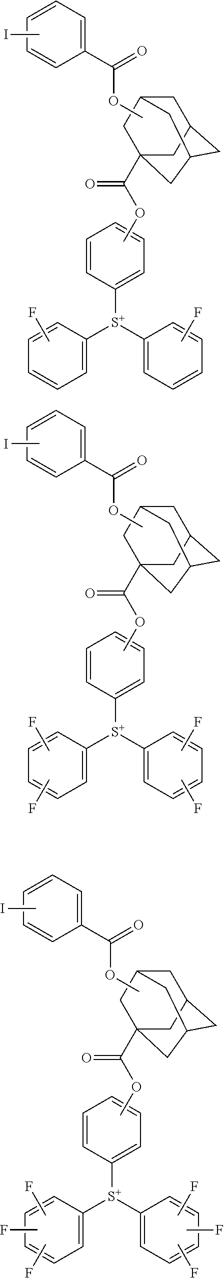

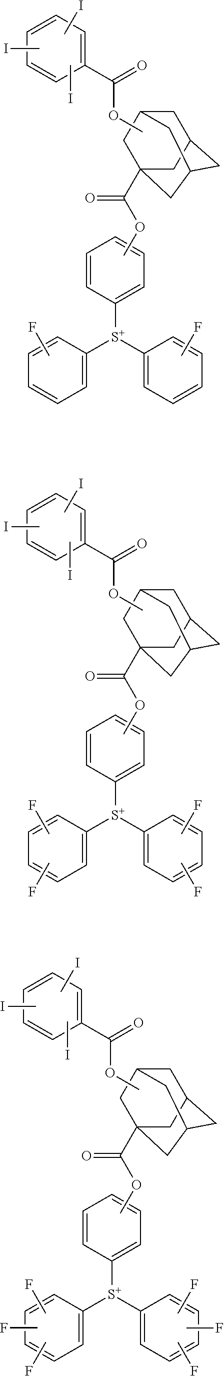

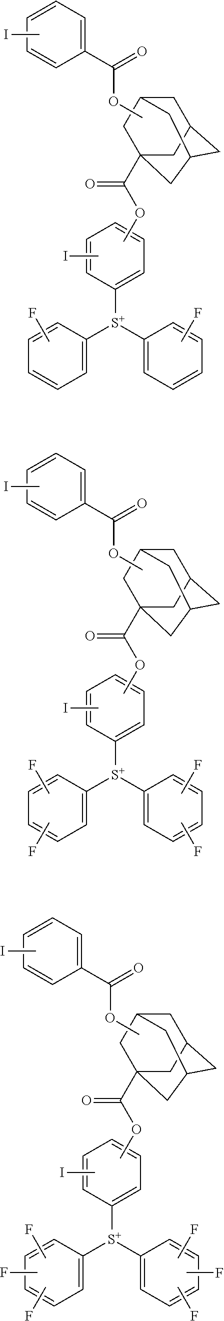

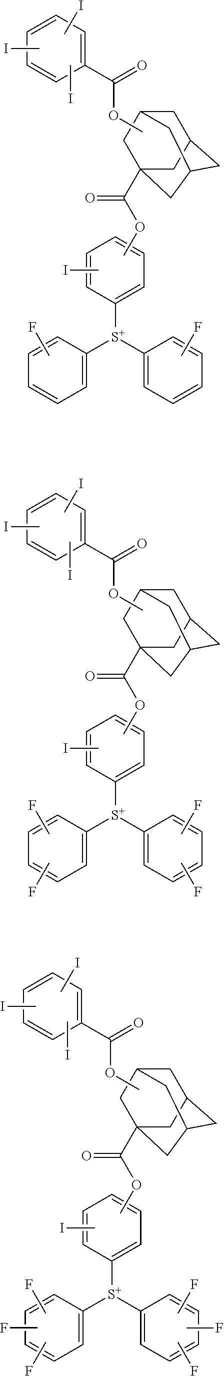

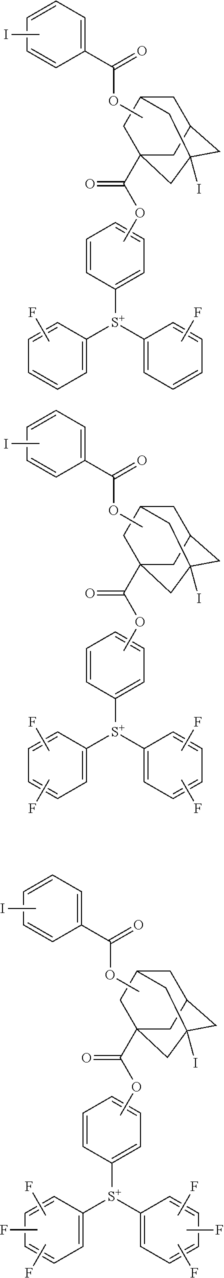

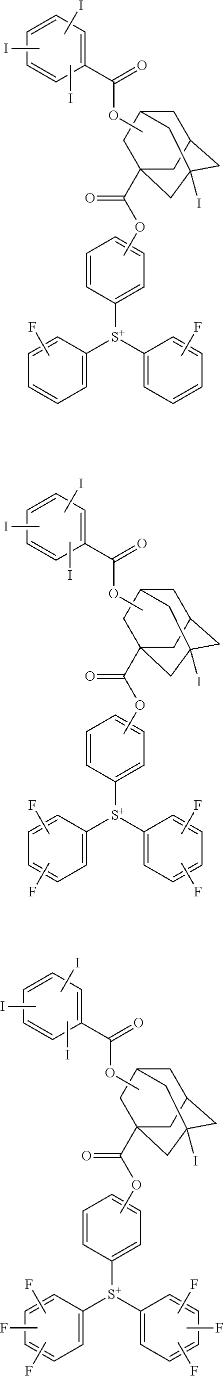

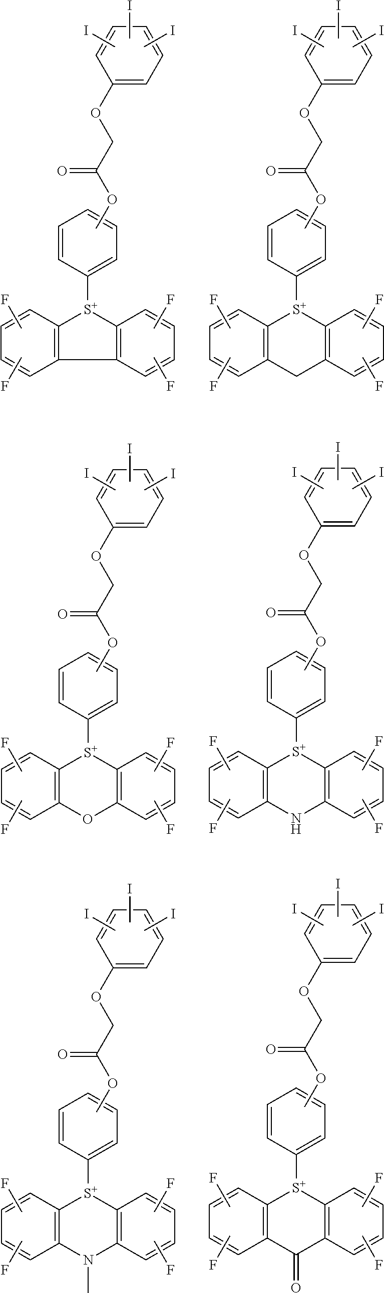

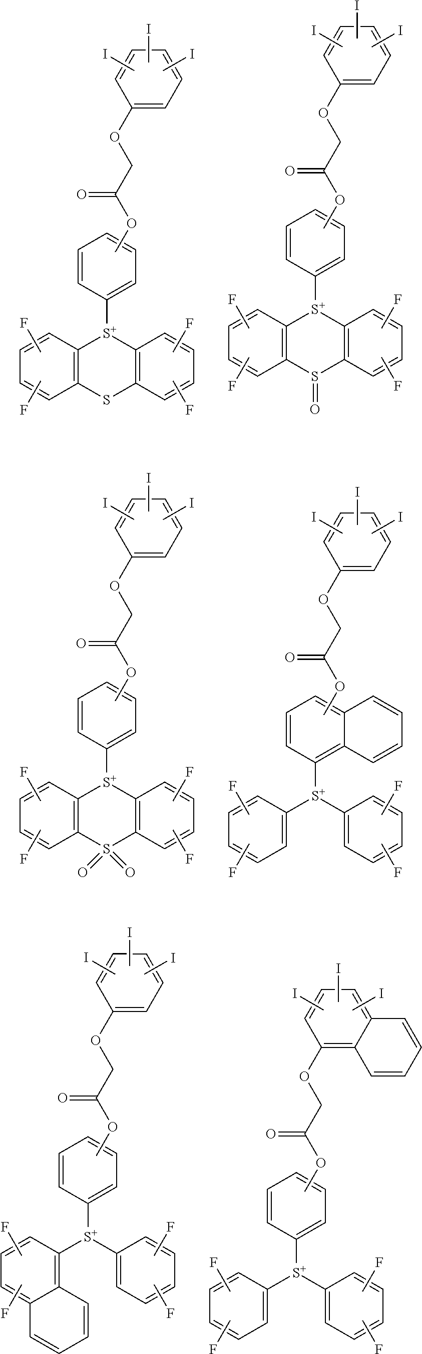

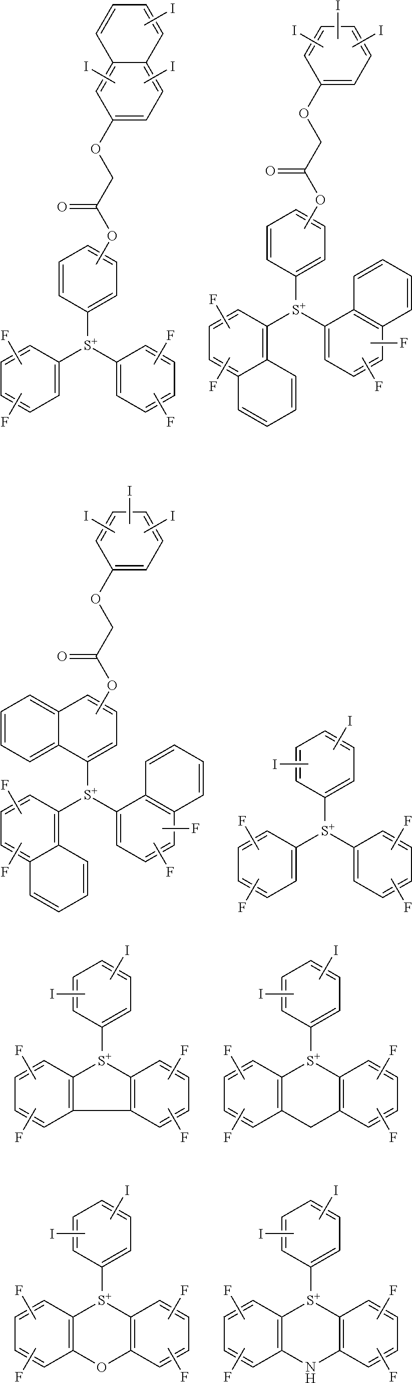

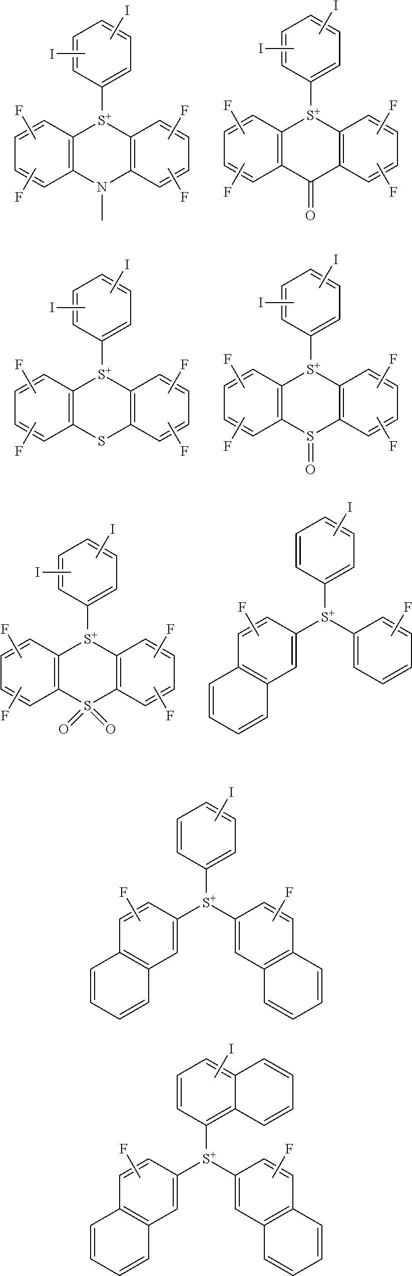

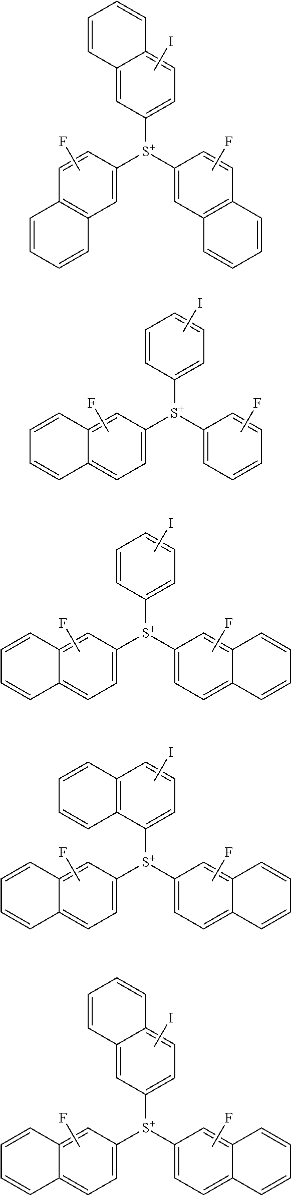

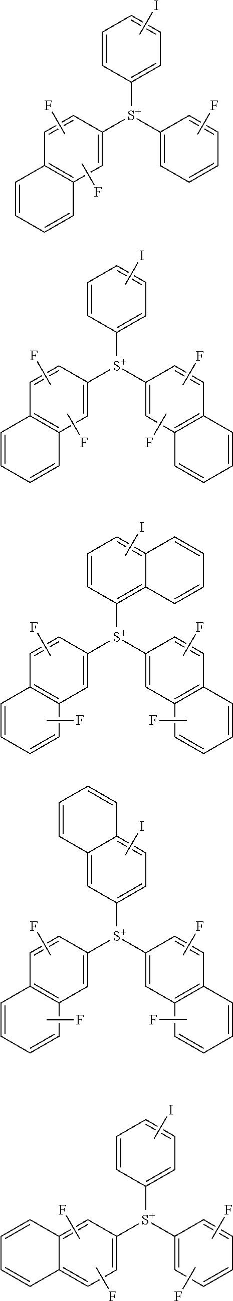

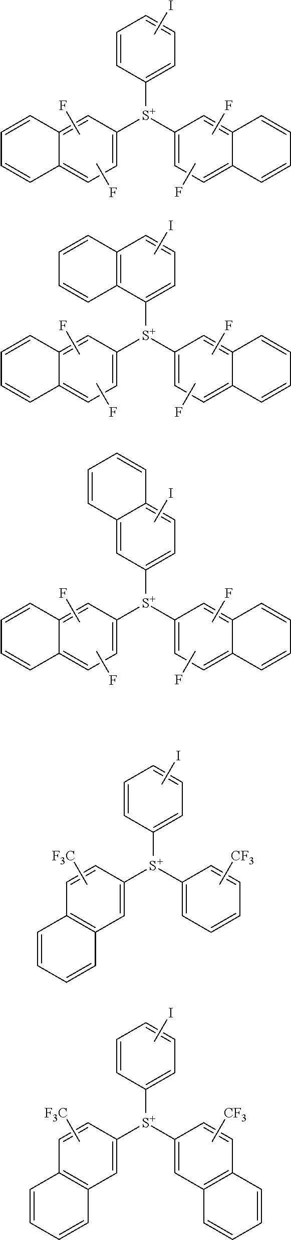

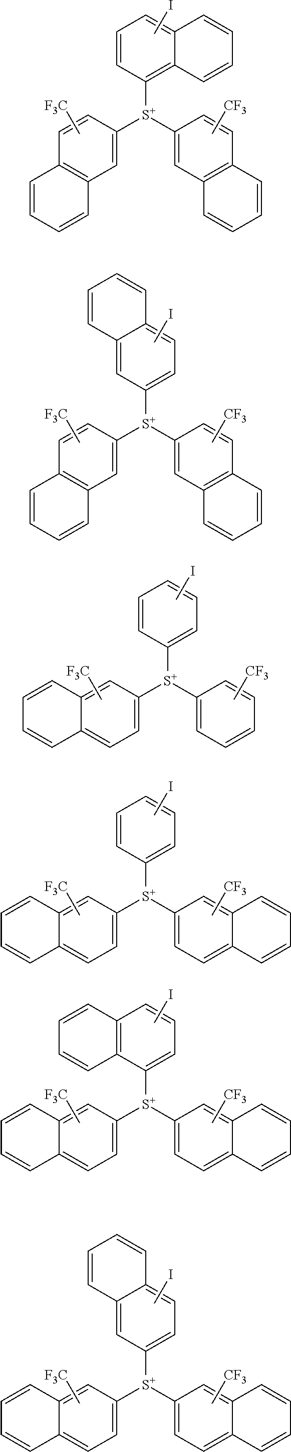

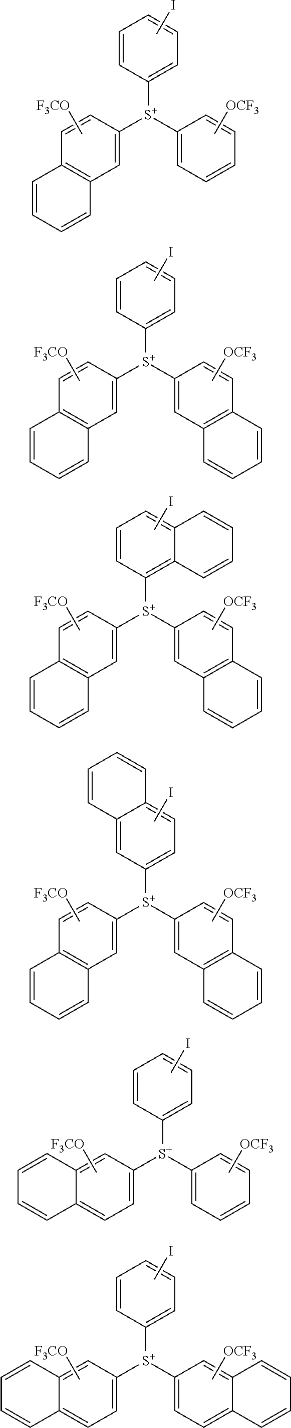

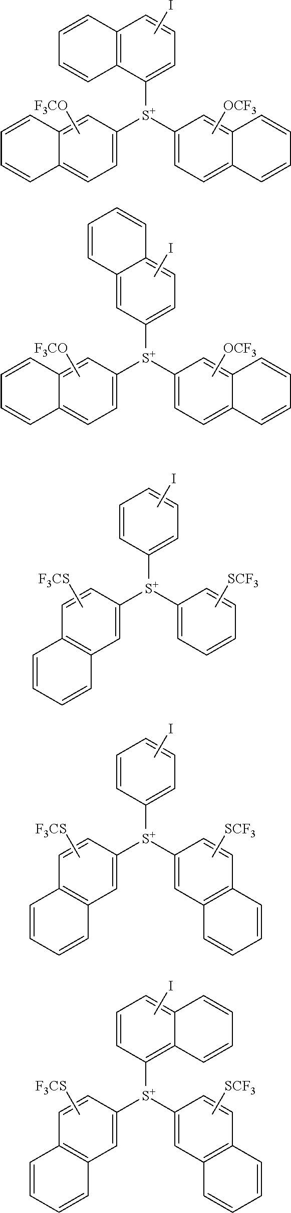

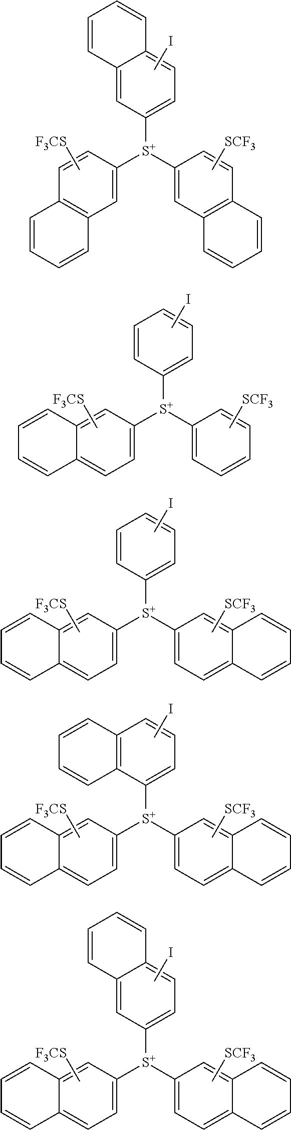

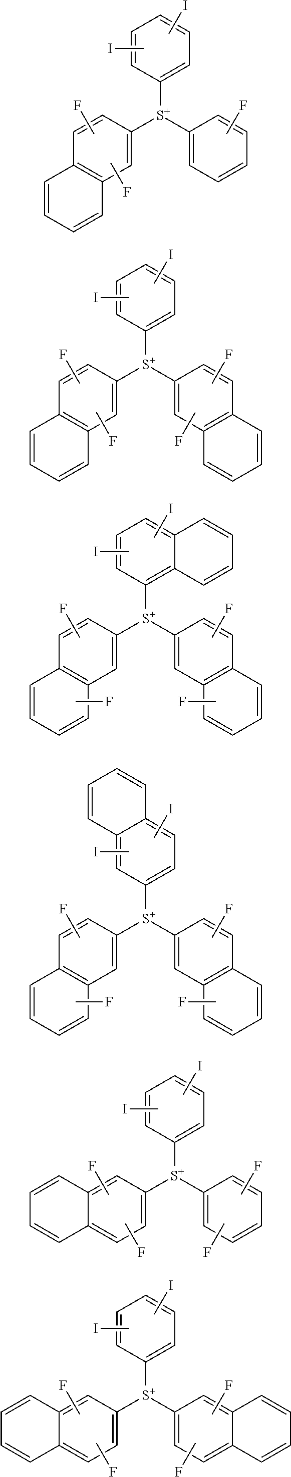

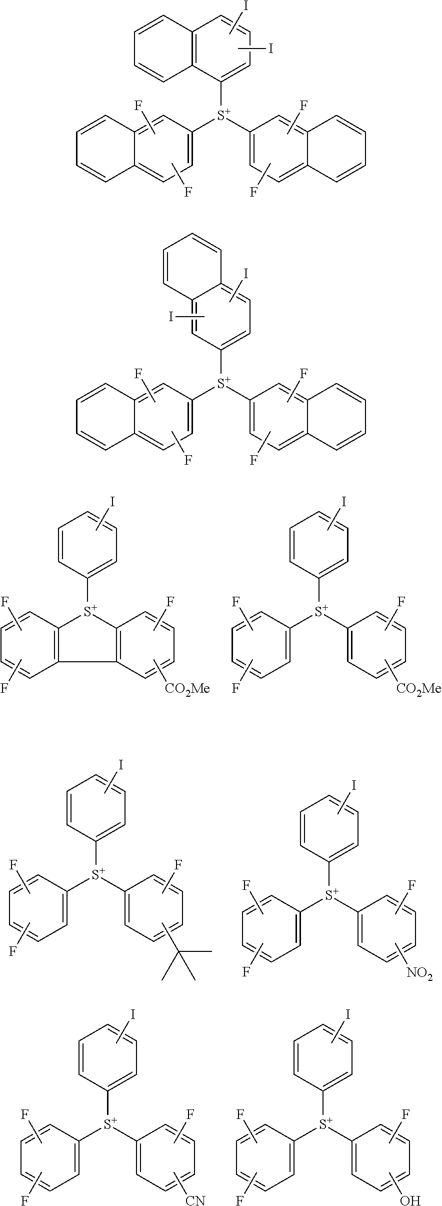

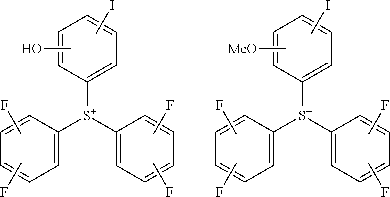

Exemplary structures of the repeat units (c1) to (c5) include arbitrary combinations of anions with cations, both as exemplified above.

Of the repeat units (c1) to (c5), repeat units (c2) to (c5) are preferred from the aspect of controlling acid diffusion, repeat units (c2), (c4) and (c5) are more preferred from the aspect of the acid strength of generated acid, and repeat units (c2) are most preferred from the aspect of solvent solubility.

The base polymer may further comprise repeat units (d) of a structure having a hydroxy group protected with an acid labile group. The repeat unit (d) is not particularly limited as long as the unit includes one or more structures having a hydroxy group protected with a protective group such that the protective group is decomposed to generate the hydroxy group under the action of acid. Repeat units having the formula (d1) are preferred.

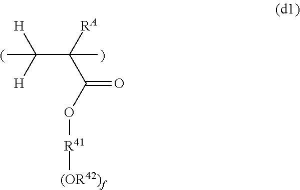

In formula (d1), R^{A} is as defined above. R⁴¹ is a C₁-C₃₀ (f+1)-valent hydrocarbon group which may contain a heteroatom. R⁴² is an acid labile group, and f is 1, 2, 3 or 4.

In formula (d1), the acid labile group R⁴² is deprotected under the action of acid so that a hydroxy group is generated. Although the structure of R⁴² is not particularly limited, an acetal structure, ketal structure, alkoxycarbonyl group and alkoxymethyl group having the following formula (d2) are preferred, with the alkoxymethyl group having formula (d2) being more preferred.

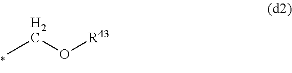

Herein R⁴³ is a C₁-C₁₅ hydrocarbyl group.

Illustrative examples of the acid labile group R⁴², the alkoxymethyl group having formula (d2), and the repeat units (d) are as exemplified for the repeat units (d) in JP-A 2020-111564 (US 20200223796).

In addition to the foregoing units, the base polymer may further comprise repeat units (e) derived from indene, benzofuran, benzothiophene, acenaphthylene, chromone, coumarin, norbornadiene and derivatives thereof. Examples of the monomer from which repeat units (e) are derived are shown below, but not limited thereto.

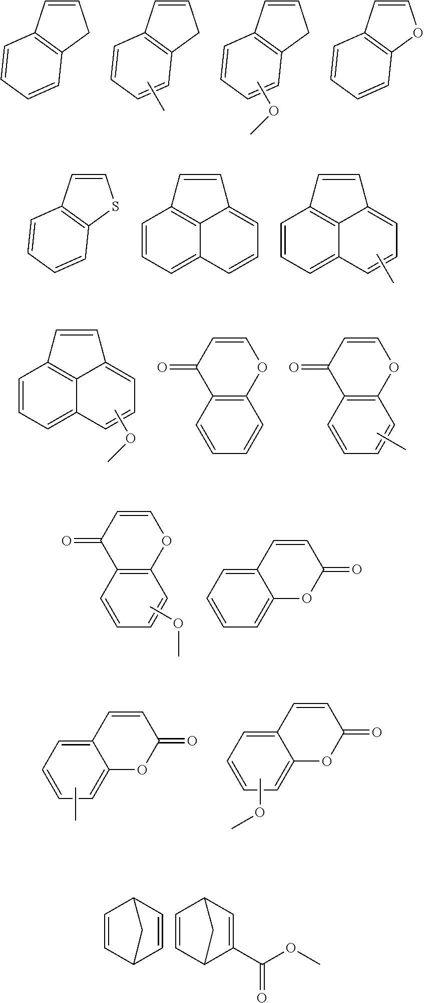

The base polymer may further comprise repeat units (f) derived from indane, vinylpyridine or vinylcarbazole.

In the polymer, repeat units (A), (a1), (a2), (a3), (b1), (b2), (c1) to (c5), (d), (e), and (f) are incorporated in a ratio of preferably 0<A≤0.5, 0≤a1≤0.8, 0≤a2≤0.8, 0≤a3≤0.6, 0≤b1≤0.6, 0≤b2≤0.6, 0≤c1≤0.4, 0≤c2≤0.4, 0≤c3≤0.4, 0≤c4≤0.4, 0≤c5≤0.4, 0≤d≤0.5, 0≤e≤0.3, and 0≤f≤0.3; more preferably 0<A≤0.4, 0≤a1≤0.7, 0≤a2≤0.7, 0≤a3≤0.5, 0b1≤0.5, 0≤b2≤0.5, 0≤c1≤0.3, 0≤c2≤0.3, 0≤c3≤0.3, 0≤c4≤0.3, 0≤c5≤0.3, 0≤d≤0.3, 0≤e≤0.3, and 0≤f≤0.3; with the proviso that A+a1+a2+a3+b1+b2+c1+c2+c3+c4+c5+d+e+f≤1.0.

The polymer should preferably have a weight average molecular weight (Mw) in the range of 1,000 to 500,000, and more preferably 3,000 to 100,000, as measured by GPC versus polystyrene standards using tetrahydrofuran (THF) or N,N-dimethylformamide (DMF) solvent. A Mw in the range ensures that the resist film has etch resistance and eliminates the risk of resolution decline by a failure to provide a difference in dissolution rate before and after exposure.

The influence of Mw/Mn becomes stronger as the pattern rule becomes finer. Therefore, the polymer should preferably have a narrow dispersity (Mw/Mn) of 1.0 to 2.0 in order to provide a resist composition suitable for micropatterning to a small feature size. A Mw/Mn in the range ensures that the contents of lower and higher molecular weight polymer fractions are low and eliminates a possibility that foreign matter is left on the pattern or the pattern profile is degraded.

The polymer may be synthesized, for example, by dissolving a monomer or monomers corresponding to the above-mentioned repeat units in an organic solvent, adding a radical polymerization initiator, and heating for polymerization.

Examples of the organic solvent which can be used for polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator used herein include 2,2′-azobisisobutyronitrile (AIBN), 2,2′-azobis(2,4-dimethylvaleronitrile), dimethyl 2,2-azobis(2-methylpropionate), 1,1′-azobis(1-acetoxy-1-phenylethane), benzoyl peroxide, and lauroyl peroxide. The initiator is preferably added in an amount of 0.01 to 25 mol % based on the total of monomers to be polymerized. The reaction temperature is preferably 50 to 150° C., more preferably 60 to 100° C. The reaction time is preferably 2 to 24 hours, more preferably 2 to 12 hours in view of production efficiency.

The polymerization initiator may be fed to the reactor either by adding the initiator to the monomer solution and feeding the solution to the reactor, or by dissolving the initiator in a solvent to form an initiator solution and feeding the initiator solution and the monomer solution independently to the reactor. Because of a possibility that in the standby duration, the initiator generates a radical which triggers polymerization reaction to form a ultra-high-molecular-weight polymer, it is preferred from the standpoint of quality control to prepare the monomer solution and the initiator solution separately and add them dropwise. The acid labile group that has been incorporated in the monomer may be kept as such, or polymerization may be followed by protection or partial protection. During the polymer synthesis, any known chain transfer agent such as dodecyl mercaptan or 2-mercaptoethanol may be added for molecular weight control purpose. The amount of chain transfer agent added is preferably 0.01 to 20 mol % based on the total of monomers.

When a hydroxy-containing monomer is copolymerized, the hydroxy group is substituted by an acetal group which is susceptible to deprotection with acid, typically ethoxyethoxy, prior to polymerization, and the polymerization is followed by deprotection with weak acid and water. Alternatively, the hydroxy group is substituted by an acetyl, formyl or pivaloyl group prior to polymerization, and the polymerization is followed by alkaline hydrolysis.

When hydroxystyrene or hydroxyvinylnaphthalene is copolymerized, one method is dissolving hydroxystyrene or hydroxyvinylnaphthalene and other monomers in an organic solvent, adding a radical polymerization initiator thereto, and heating the solution for polymerization. In an alternative method, acetoxystyrene or acetoxyvinylnaphthalene is used instead, and after polymerization, the acetoxy group is deprotected by alkaline hydrolysis, for thereby converting the polymer product to polyhydroxystyrene or polyhydroxyvinylnaphthalene.

For alkaline hydrolysis, a base such as aqueous ammonia or triethylamine may be used. Preferably the reaction temperature is −20° C. to 100° C., more preferably 0° C. to 60° C., and the reaction time is 0.2 to 100 hours, more preferably 0.5 to 20 hours.

The amounts of monomers in the monomer solution may be determined appropriate so as to provide the preferred fractions of repeat units.

It is now described how to use the polymer obtained by the above preparation method. The reaction solution resulting from polymerization reaction may be used as the final product. Alternatively, the polymer may be recovered in powder form through a purifying step such as re-precipitation step of adding the polymerization solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145](U.S. Pat. No. 7,537,880). Exemplary solvents include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as γ-butyrolactone (GBL); and high-boiling alcohols such as diethylene glycol, propylene glycol, glycerol, 1,4-butanediol, and 1,3-butanediol, which may be used alone or in admixture.

The polymer solution preferably has a polymer concentration of 0.01 to 30% by weight, more preferably 0.1 to 20% by weight.

Prior to use, the reaction solution or polymer solution is preferably filtered through a filter. Filtration is effective for consistent quality because foreign particles and gel which can cause defects are removed.

Suitable materials of which the filter is made include fluorocarbon, cellulose, nylon, polyester, and hydrocarbon base materials. Preferred for the filtration of a resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

### [Chemically Amplified Resist Composition]

### (A) Base Polymer

Another embodiment of the invention is a chemically amplified resist composition comprising (A) a base polymer containing the polymer defined above.

The base polymer (A) may be used alone or as a blend of two or more polymers which differ in compositional ratio, Mw and/or Mw/Mn. Component (A) may also be a blend of the polymer defined above and a hydrogenated product of ROMP. For the ROMP, reference is made to JP-A 2003-066612.

### (B) Organic Solvent

The resist composition may comprise an organic solvent as component (B). The organic solvent (B) is not particularly limited as long as the foregoing and other components are soluble therein. Suitable solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate (EL), ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone (GBL), and mixtures thereof.

Of the foregoing organic solvents, it is recommended to use 1-ethoxy-2-propanol, PGMEA, cyclohexanone, GBL, EL, DAA and mixtures thereof because the base polymer (A) is most soluble therein.

The organic solvent (B) is preferably added in an amount of 200 to 8,000 parts by weight, and more preferably 400 to 6,000 parts by weight per 80 parts by weight of the base polymer (A). The organic solvent may be used alone or in admixture.

### (C) Quencher

The resist composition may further comprise (C) a quencher. As used herein, the “quencher” refers to a compound capable of trapping the strong acid generated by the PAG to prevent the acid from diffusing into the unexposed region of resist film, for forming the desired pattern. As used herein, the strong acid refers to an acid having a sufficient acidity to trigger the deprotection reaction of an acid labile group.

Preferred examples of the quencher include onium salts having the formulae (1) and (2).

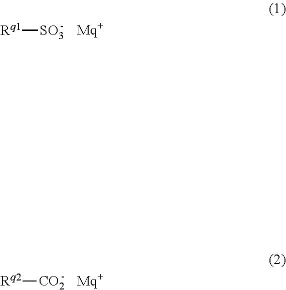

In formula (1), R^{q1} is hydrogen or a C₁-C₄₀ hydrocarbyl group which may contain a heteroatom, exclusive of the group wherein hydrogen bonded to the carbon atom at α-position relative to the sulfo group is substituted by fluorine or fluoroalkyl. In formula (2), R^{q2} is hydrogen or a C₁-C₄₀ hydrocarbyl group which may contain a heteroatom.

Examples of the C₁-C₄₀ hydrocarbyl group R^{q1} include C₁-C₄₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decyl, and adamantyl; C₆-C₄₀ aryl groups such as phenyl, naphthyl and anthracenyl. In the hydrocarbyl group, some or all hydrogen may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonic ester bond, carbonate bond, lactone ring, sultone ring, carboxylic anhydride (—C(═O)—O—C(═O)—), or haloalkyl moiety.

Examples of the hydrocarbyl group R^{q2} include those exemplified above for R^{q1} and fluorinated saturated hydrocarbyl groups, for example, fluorinated alkyl groups such as trifluoromethyl and trifluoroethyl, and fluorinated aryl groups such as pentafluorophenyl and 4-trifluoromethylphenyl.

Examples of the anion in the onium salt having formula (1) are shown below, but not limited thereto.

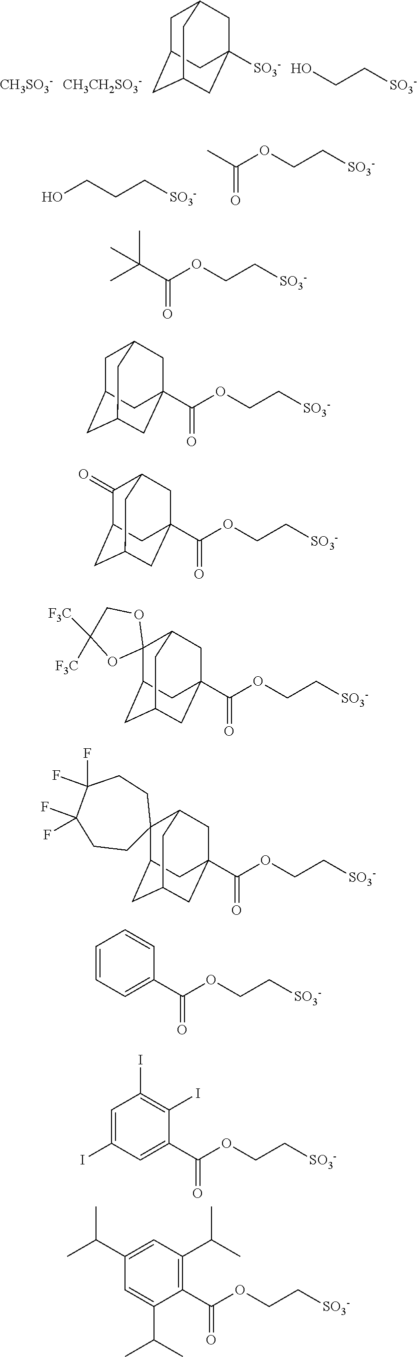

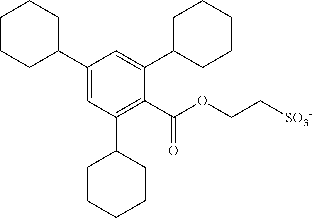

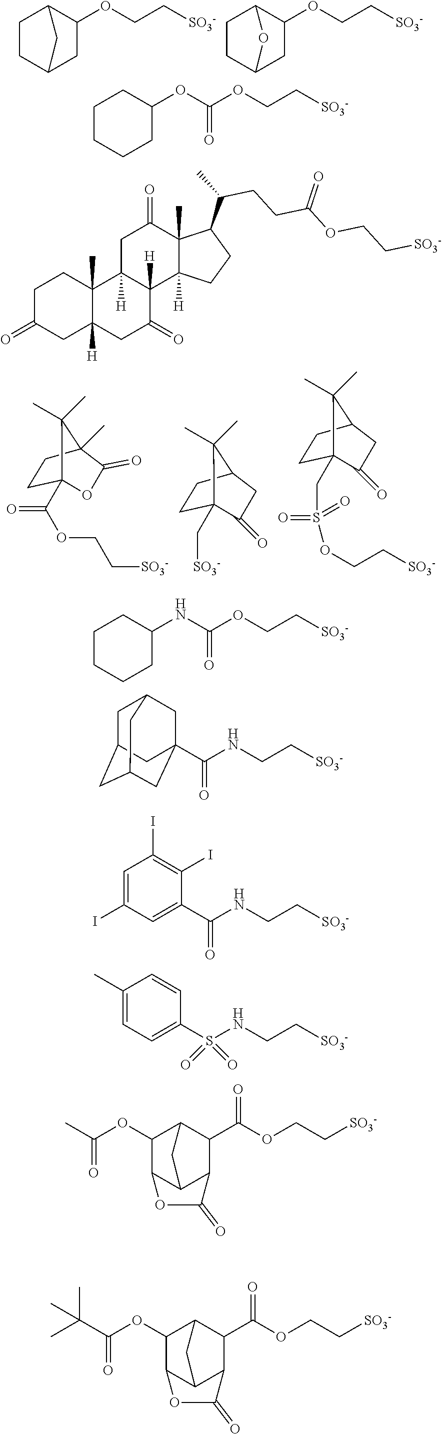

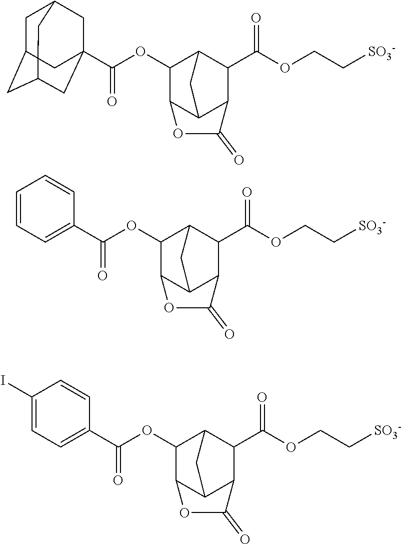

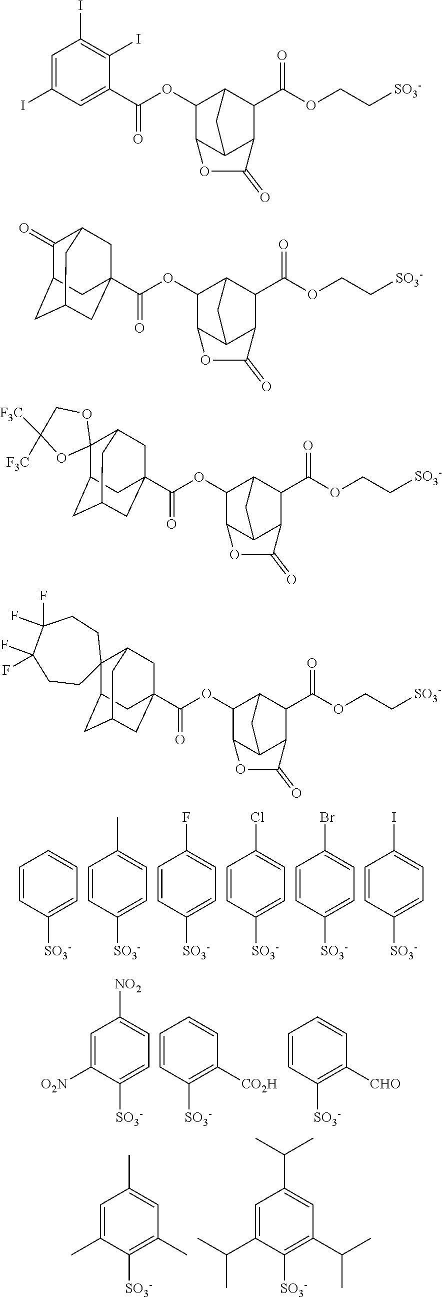

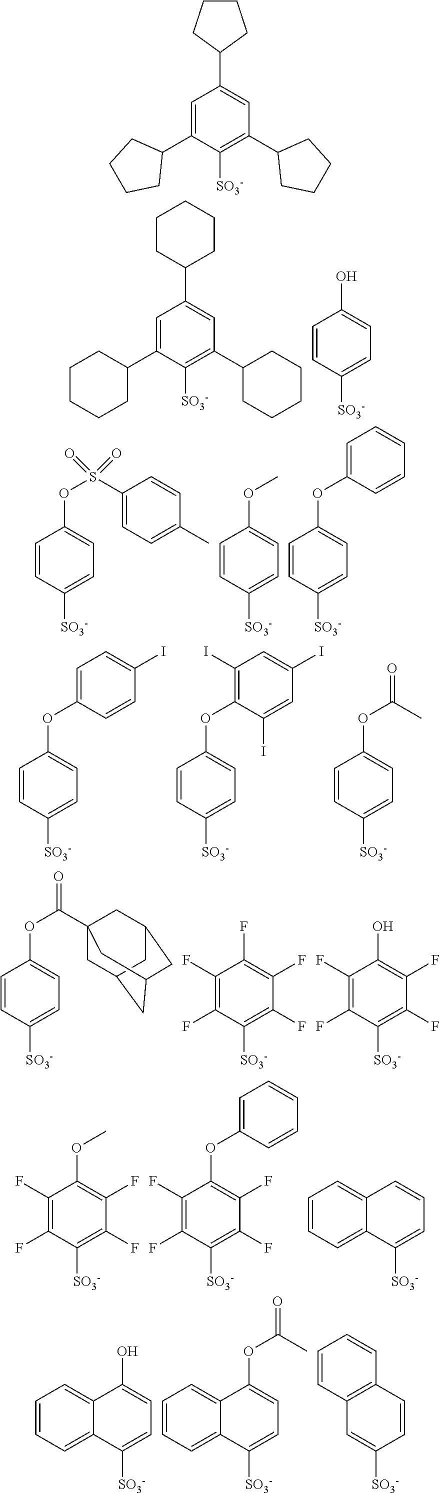

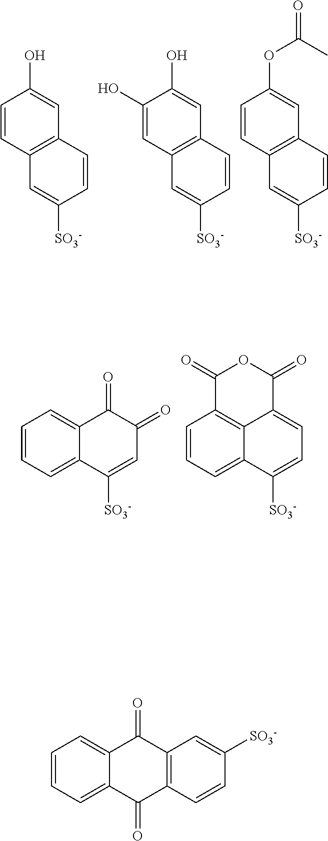

Examples of the anion in the onium salt having formula (2) are shown below, but not limited thereto.

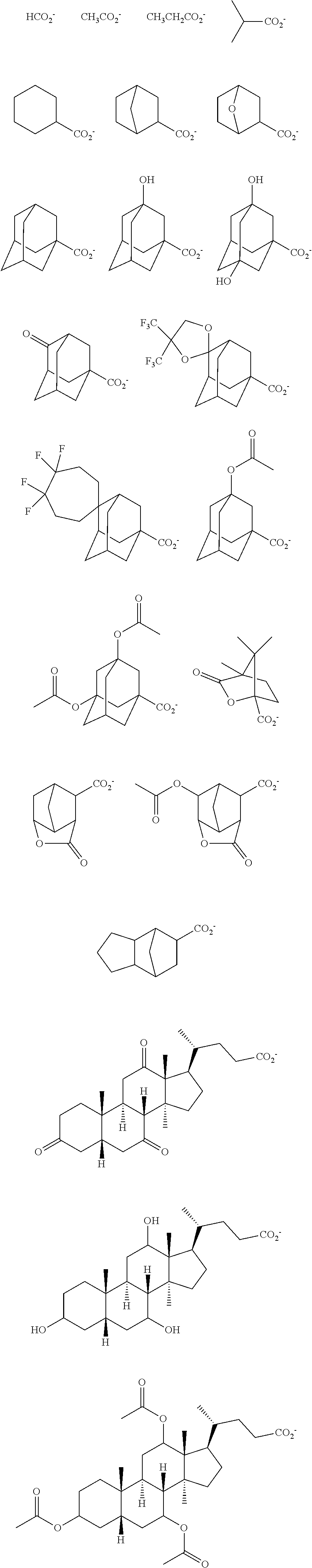

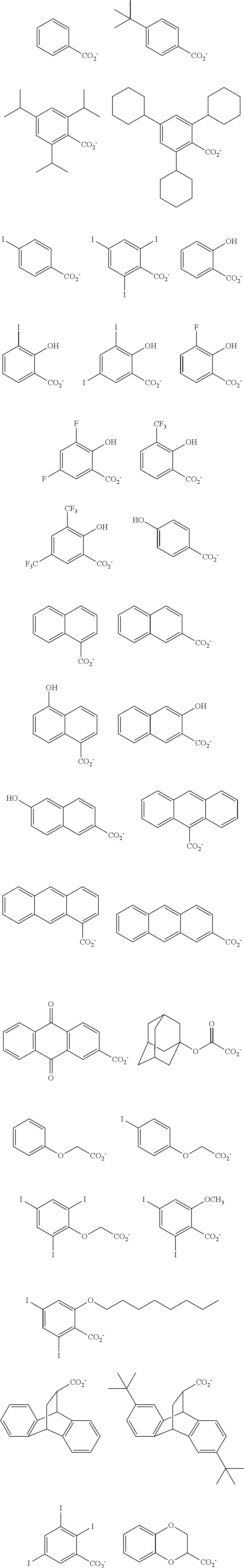

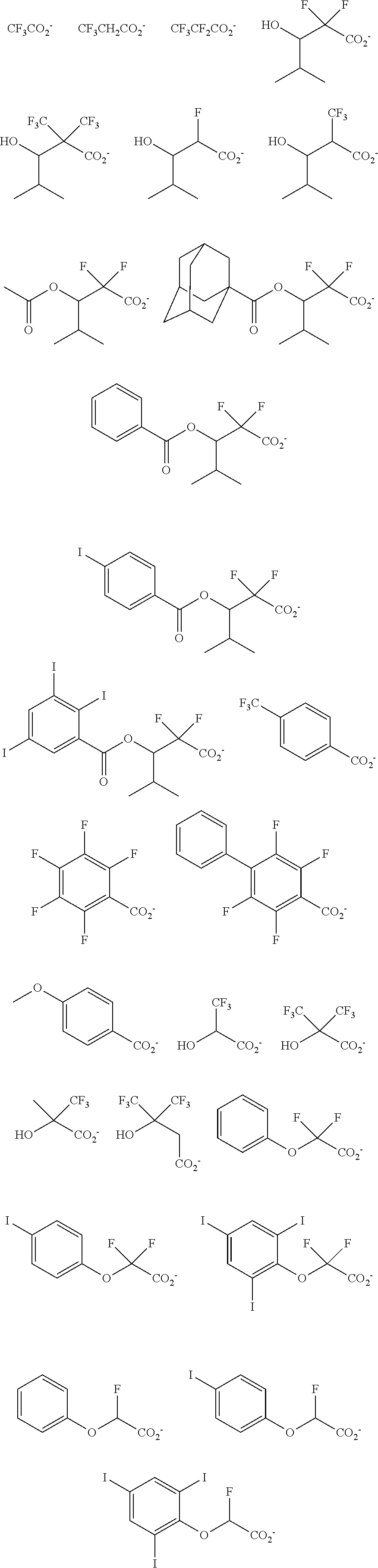

In formulae (1) and (2), Mq⁺ is an onium cation. Suitable onium cations include sulfonium, iodonium and ammonium cations. Examples of the sulfonium cation include those exemplified above for the sulfonium cation having formula (Z-1) and the sulfonium cation having formula (Z-3) as well as those described in JP-A 2024-003744, paragraphs [0102]-[0125], WO 2024/128017, paragraphs [0044]-[0049], and JP 7491173, paragraphs [0035]-[0046], but are not limited thereto.

Examples of the iodonium cation include those described in JP-A 2024-000259, paragraph [0181], but are not limited thereto.

Examples of the ammonium cation include those having the formula (am-1).

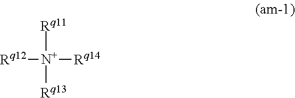

In formula (am-1), R^{q11} to R^{q14} are each independently a C₁-C₄₀ hydrocarbyl group which may contain a heteroatom. A pair of R^{q11} and R^{q12} may bond together to form a ring with the nitrogen atom to which they are attached. Examples of the hydrocarbyl group are as exemplified above for the hydrocarbyl group R¹ in formula (A).

Examples of the ammonium cation having formula (am-1) are shown below, but not limited thereto.

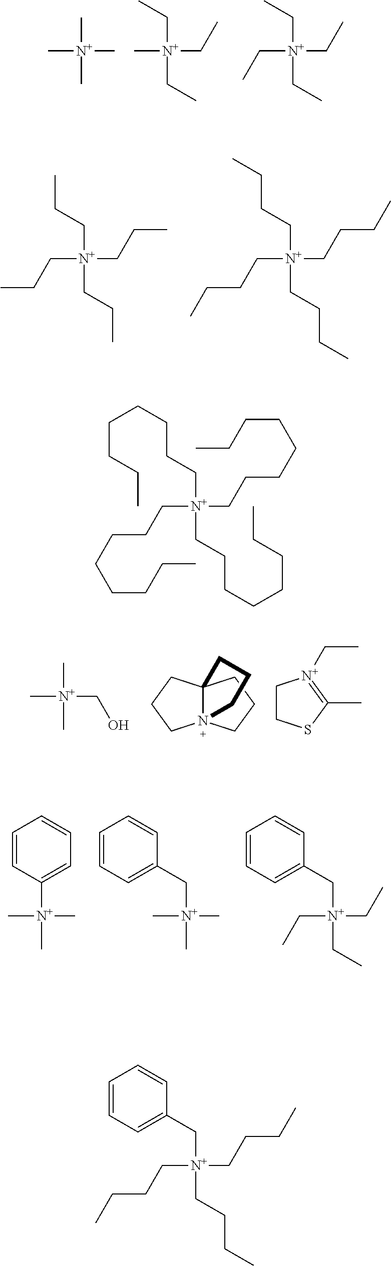

Examples of the onium salt having formula (1) or (2) include arbitrary combinations of anions with cations, both as exemplified above. These onium salts may be readily synthesized by ion exchange reaction according to any well-known organic chemistry technique. For the ion exchange reaction, reference may be made to JP-A 2007-145797, for example.

The onium salt having formula (1) or (2) functions as a quencher in the resist composition because the counter anion of the onium salt is a conjugated base of a weak acid. As used herein, the weak acid indicates an acidity insufficient to deprotect an acid labile group from an acid labile group-containing unit in the base polymer. The onium salt having formula (1) or (2) functions as a quencher when used in combination with an onium salt type PAG having a conjugated base of a strong acid (typically α-fluorinated sulfonic acid) as the counter anion. In a system using a mixture of an onium salt capable of generating a strong acid (e.g., α-fluorinated sulfonic acid) and an onium salt capable of generating a weak acid (e.g., non-fluorinated sulfonic acid or carboxylic acid), if the strong acid generated from the PAG upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling to control acid diffusion.

Also useful as the quencher (C) are onium salts having a sulfonium cation and a phenoxide anion site in a common molecule as described in JP 6848776, onium salts having a sulfonium cation and a carboxylate anion site in a common molecule as described in JP 6583136 and JP-A 2020-200311, and onium salts having an iodonium cation and a carboxylate anion site in a common molecule as described in JP 6274755.

If a PAG capable of generating a strong acid is an onium salt, an exchange from the strong acid generated upon exposure to high-energy radiation to a weak acid as above can take place, but it rarely happens that the weak acid generated upon exposure to high-energy radiation collides with the unreacted onium salt capable of generating a strong acid to induce a salt exchange. This is because of a likelihood of an onium cation forming an ion pair with a stronger acid anion.

When the onium salt having formula (1) or (2) is used as the quencher (C), the amount of the onium salt used is preferably 0.1 to 20 parts by weight, more preferably 0.1 to 10 parts by weight per 80 parts by weight of the base polymer (A). As long as the amount of component (C) is in the range, a satisfactory resolution is available without a substantial lowering of sensitivity. The onium salt having formula (1) or (2) may be used alone or in admixture.

Nitrogen-containing compounds may also be used as the quencher (C). Suitable nitrogen-containing compounds include primary, secondary and tertiary amine compounds, specifically amine compounds having a hydroxy group, ether bond, ester bond, lactone ring, cyano group or sulfonic ester bond, as described in JP-A 2008-111103, paragraphs [0146]-[0164](U.S. Pat. No. 7,537,880), and primary or secondary amine compounds protected with a carbamate group, as described in JP 3790649.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-046501, for example.

When the nitrogen-containing compound is used as the quencher (C), the amount of the nitrogen-containing compound used is preferably 0.001 to 12 parts by weight, more preferably 0.01 to 8 parts by weight per 80 parts by weight of the base polymer (A). The nitrogen-containing compound may be used alone or in admixture.

### (D) Photoacid Generator

The chemically amplified resist composition may comprise (D) a photoacid generator. The PAG is not particularly limited as long as it is capable of generating an acid upon exposure to high-energy radiation.

The preferred PAG is a salt having the formula (3) or (4).

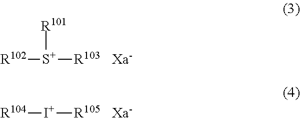

In formula (3), R¹⁰¹ to R¹⁰⁵ are each independently halogen or a C₁-C₂₀ hydrocarbyl group which may contain a heteroatom. Any two of R¹⁰¹, R¹⁰² and R¹⁰³ may bond together to form a ring with the sulfur atom to which they are attached.

Examples of the cation in the sulfonium and iodonium salts having formulae (3) and (4) are as exemplified above for the sulfonium and iodonium cations A⁺ in formulae (c2) to (c5), but not limited thereto.

In formulae (3) and (4), Xa⁻ is an anion of strong acid selected from formulae (c1-1) to (c1-5).

Compounds having the formula (5) are also preferred as the PAG (D).

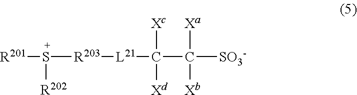

In formula (5), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰² and R²⁰³ may bond together to form a ring with the sulfur atom to which they are attached.

The C₁-C₃₀ hydrocarbyl group represented by R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl, and adamantyl; and C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl, and combinations thereof. In these hydrocarbyl groups, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety.

The C₁-C₃₀ hydrocarbylene group R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbornanediyl and adamantanediyl; and arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, and tert-butylnaphthylene. In these hydrocarbylene groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety. Of the heteroatoms, oxygen is preferred.

In formula (5), L²¹ is a single bond, ether bond or a C₁-C₂₀ hydrocarbylene group which may contain a heteroatom. The hydrocarbylene group may be saturated or unsaturated and straight, branched or cyclic. Examples thereof are as exemplified above for the hydrocarbylene group R²⁰³.

In formula (5), X^{a}, X^{b}, X^{c} and X^{d} are each independently hydrogen, fluorine or trifluoromethyl, at least one of X^{a}, X^{b}, X^{c} and X^{d} being fluorine or trifluoromethyl.

Of the PAGs having formula (5), those having formula (5′) are preferred.

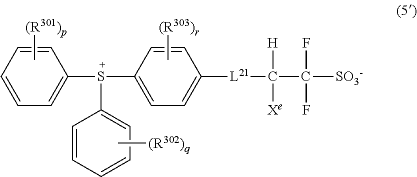

In formula (5′), L²¹ is as defined above. X^{e} is hydrogen or trifluoromethyl, preferably trifluoromethyl. R³⁰¹, R³⁰² and R³⁰³ are each independently hydrogen or a C₁-C₂₀ hydrocarbyl group which may contain a heteroatom. The hydrocarbyl group may be saturated or unsaturated and straight, branched or cyclic. Examples thereof are as exemplified above for R^{fa1} in formula (c1-1-1). The subscripts p and q are each independently 0, 1, 2, 3, 4 or 5, and r is 0, 1, 2, 3 or 4.

Examples of the PAG having formula (5) include those exemplified for the PAG having formula (2) in JP-A 2017-026980.

Of the foregoing PAGs, those having an anion of formula (c1-1-1) or (c1-4) are especially preferred because of reduced acid diffusion and high solubility in solvents. Also those having formula (5′) are especially preferred because of extremely reduced acid diffusion.

When used, the PAG (D) is preferably added in an amount of 0.1 to 40 parts, and more preferably 0.5 to 20 parts by weight per 80 parts by weight of the base polymer (A). As long as the amount of the PAG is in the range, good resolution is achievable and the risk of foreign particles being formed after development or during stripping of resist film is avoided. The PAG may be used alone or in admixture.

### (E) Surfactant

The resist composition may further include (E) a surfactant. Preferred are a surfactant which is insoluble or substantially insoluble in water and soluble in alkaline developer, and a surfactant which is insoluble or substantially insoluble in water and alkaline developer. For the surfactant, reference should be made to those compounds described in JP-A 2010-215608 and JP-A 2011-016746.

While many examples of the surfactant which is insoluble or substantially insoluble in water and alkaline developer are described in the patent documents cited herein, preferred examples are surfactants FC-4430 (3M), Olfine® E1004 (Nissin Chemical Co., Ltd.), Surflon® 5-381, KH-20 and KH-30 (AGC Seimi Chemical Co., Ltd.). Partially fluorinated oxetane ring-opened polymers having the formula (surf-1) are also useful.

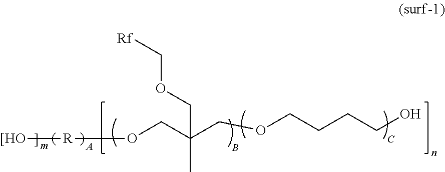

It is provided herein that R, Rf, A, B, C, m, and n are applied to only formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

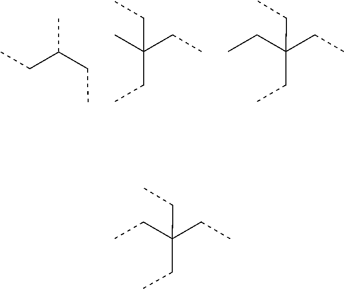

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferably used.

Rf is trifluoromethyl or pentafluoroethyl, and preferably trifluoromethyl. The letter m is an integer of 0 to 3, n is an integer of 1 to 4, and the sum of m and n, which represents the valence of R, is an integer of 2 to 4. “A” is equal to 1, B is an integer of 2 to 25, and C is an integer of 0 to 10. Preferably, B is an integer of 4 to 20, and C is 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist surface for achieving a function of minimizing water penetration or leaching. The surfactant is also effective for preventing water-soluble components from being leached out of the resist film for minimizing any damage to the exposure tool. The surfactant becomes solubilized during aqueous alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing repeat units of at least one type selected from the formulae (6A) to (6E).

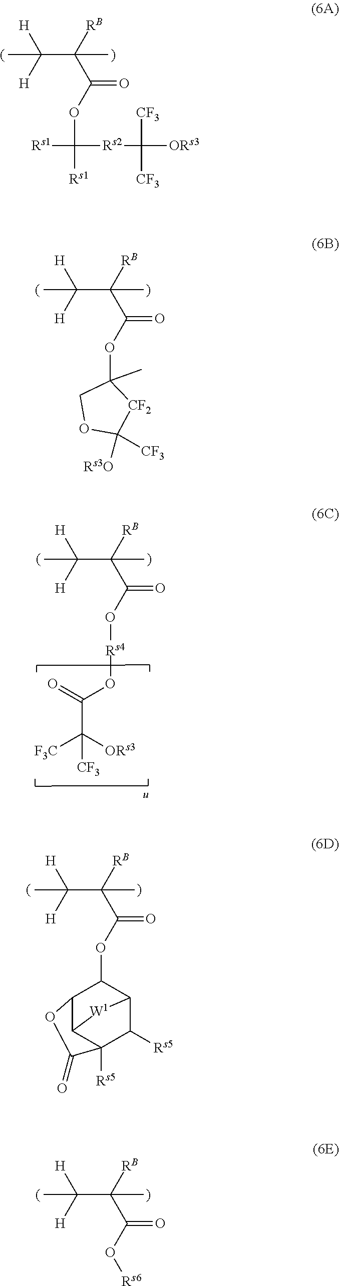

Herein, R^{B} is hydrogen, fluorine, methyl or trifluoromethyl. W¹ is —CH₂—, —CH₂CH₂— or —O—, or two separate —H. R^{s1} is each independently hydrogen or a C₁-C₁₀ hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group, and u is 1, 2 or 3. R^{s5} is each independently hydrogen or a group: —C(═O)—O—R^{sa} wherein R^{sa} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The hydrocarbyl group represented by R^{s1} may be straight, branched or cyclic and is preferably saturated. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl, n-decyl, and C₃-C₁₀ cyclic saturated hydrocarbyl groups such as cyclopropyl, cyclobutyl, cyclopentyl, cyclohexyl, adamantyl, and norbornyl. Inter alia, C₁-C₆ hydrocarbyl groups are preferred.

The hydrocarbylene group represented by R^{s2} may be straight, branched or cyclic and is preferably saturated. Examples thereof include methylene, ethylene, propylene, butylene and pentylene.

The hydrocarbyl group represented by R^{s3} or R^{S6} may be saturated or unsaturated and straight, branched or cyclic. Examples thereof include saturated hydrocarbyl groups, and aliphatic unsaturated hydrocarbyl groups such as alkenyl and alkynyl groups, with the saturated hydrocarbyl groups being preferred. Suitable saturated hydrocarbyl groups include those exemplified for the hydrocarbyl group represented by R^{s1} as well as undecyl, dodecyl, tridecyl, tetradecyl, and pentadecyl. Examples of the fluorinated hydrocarbyl group represented by R^{s3} or R^{S6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group represented by R^{s3} include groups of the above formulae (AL-3) to (AL-5), trialkylsilyl groups in which each alkyl moiety has 1 to 6 carbon atoms, and C₄-C₂₀ oxoalkyl groups.

The (u+1)-valent hydrocarbon or fluorinated hydrocarbon group represented by R^{S4} may be straight, branched or cyclic and examples thereof include the foregoing hydrocarbyl or fluorinated hydrocarbyl groups from which u number of hydrogen atoms are eliminated.

The fluorinated hydrocarbyl group represented by R^{sa} may be straight, branched or cyclic and is preferably saturated. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units having formulae (6A) to (6E) are shown below, but not limited thereto. Herein R^{B} is as defined above.

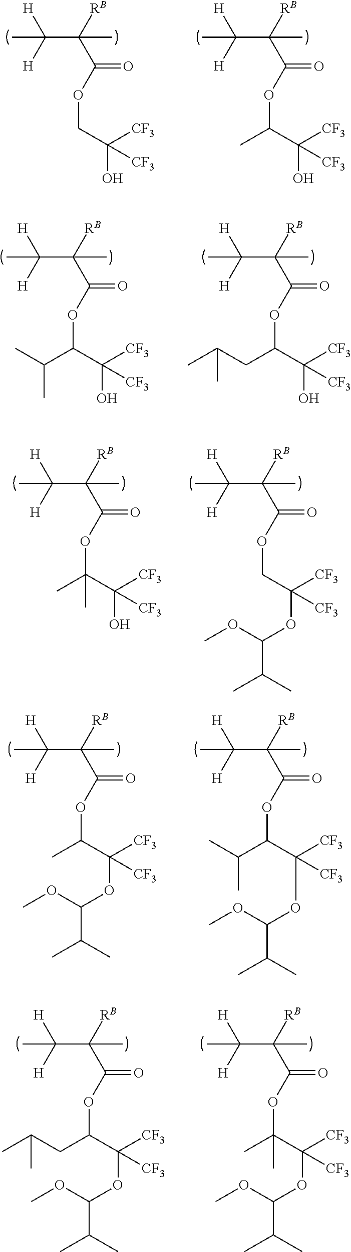

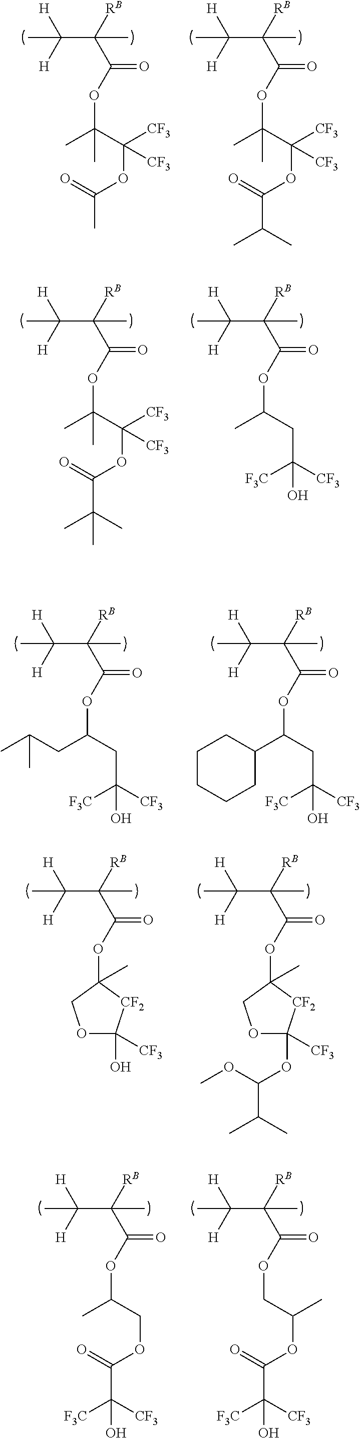

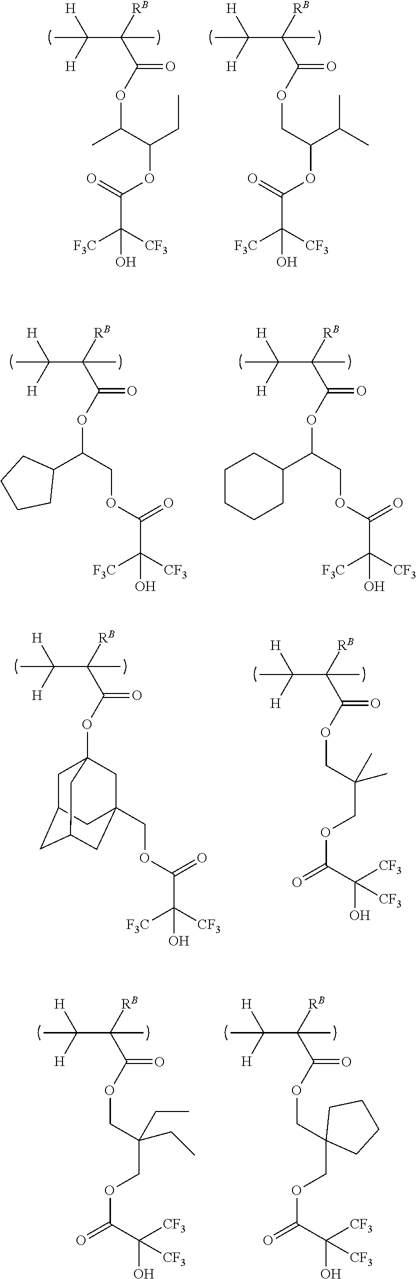

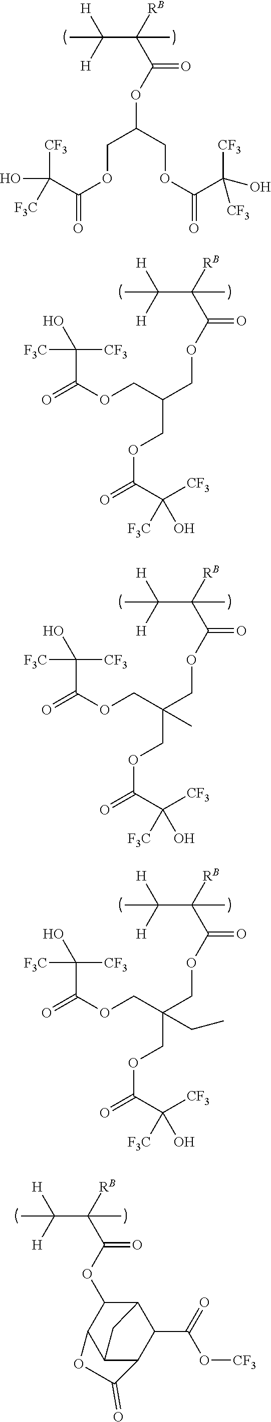

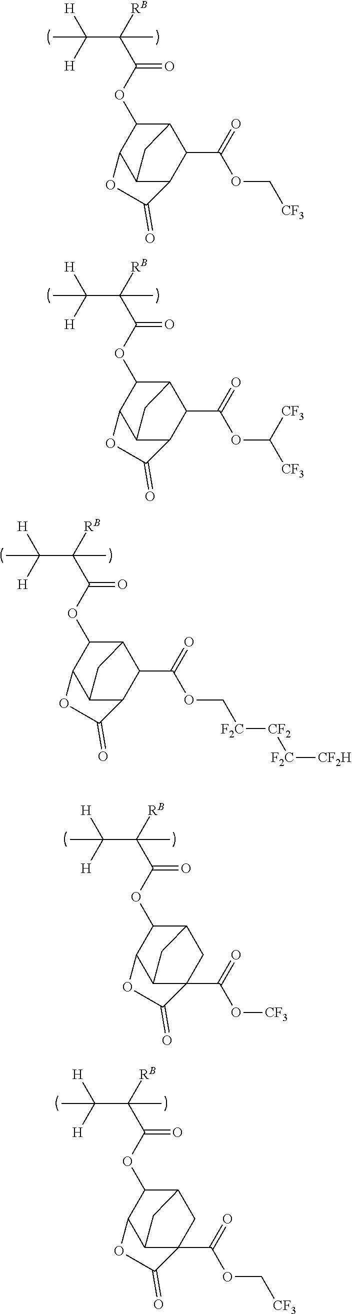

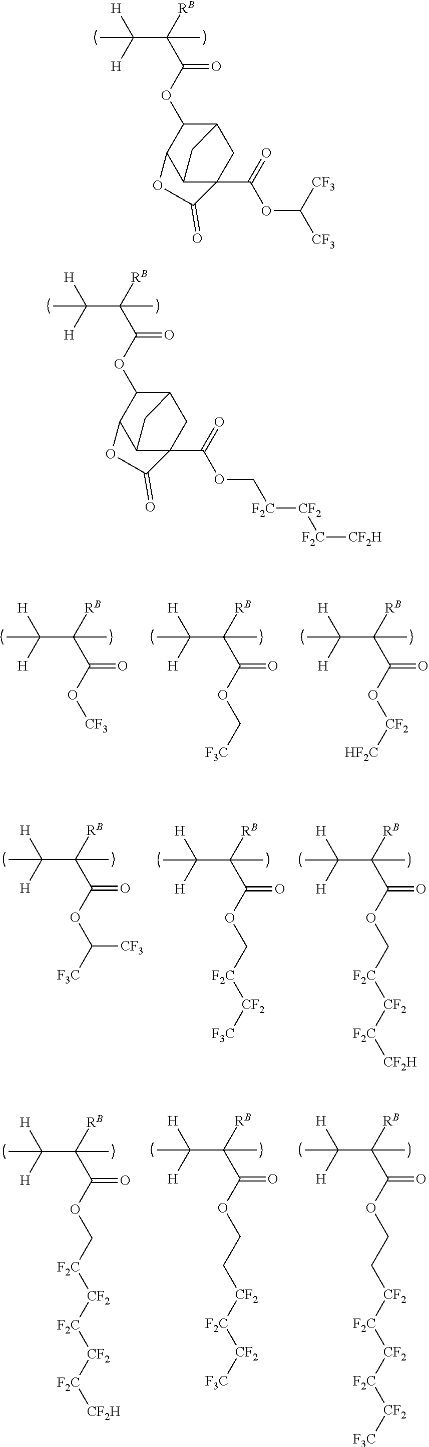

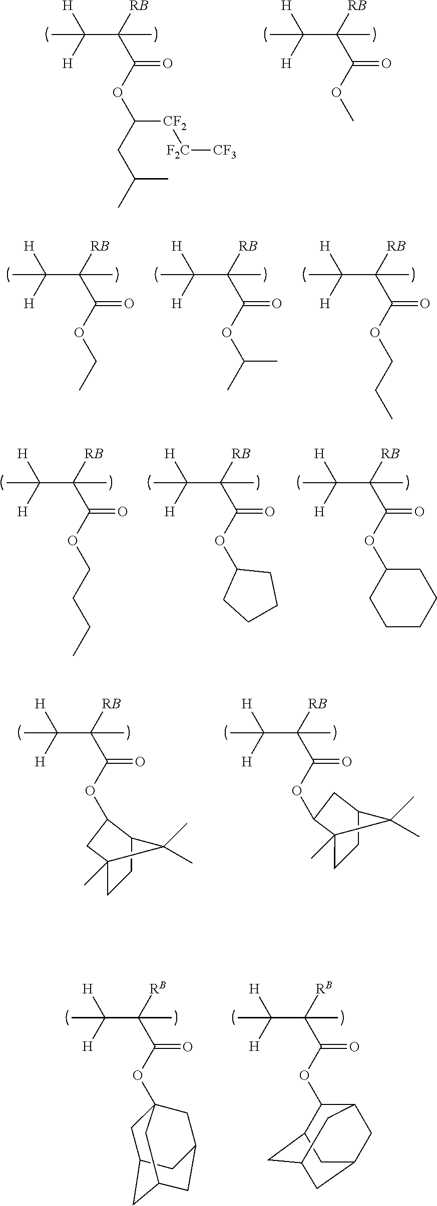

The polymeric surfactant may further contain repeat units other than the repeat units having formulae (6A) to (6E). Typical other repeat units are those derived from methacrylic acid and α-trifluoromethylacrylic acid derivatives. In the polymeric surfactant, the content of the repeat units having formulae (6A) to (6E) is preferably at least 20 mol %, more preferably at least 60 mol %, most preferably 100 mol % of the overall repeat units.

The polymeric surfactant preferably has a Mw of 1,000 to 500,000, more preferably 3,000 to 100,000 and a Mw/Mn of 1.0 to 2.0, more preferably 1.0 to 1.6.

The polymeric surfactant may be synthesized by any desired method, for example, by dissolving an unsaturated bond-containing monomer or monomers providing repeat units having formula (6A) to (6E) and optionally other repeat units in an organic solvent, adding a radical initiator, and heating for polymerization. Suitable organic solvents used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. Preferably the reaction temperature is 50 to 100° C. and the reaction time is 4 to 24 hours. The acid labile group that has been incorporated in the monomer may be kept as such, or the polymer may be protected or partially protected therewith at the end of polymerization.

During the synthesis of polymeric surfactant, any known chain transfer agent such as dodecyl mercaptan or 2-mercaptoethanol may be added for molecular weight control purpose. The amount of chain transfer agent added is preferably 0.01 to 10 mol % based on the total moles of monomers to be polymerized.

When the resist composition contains a surfactant (E), the amount thereof is preferably 0.1 to 50 parts by weight, and more preferably 0.5 to 10 parts by weight per 80 parts by weight of the base polymer (A). At least 0.1 part of the surfactant is effective in improving the receding contact angle with water of the resist film at its surface. Up to 50 parts of the surfactant is effective in forming a resist film having a low rate of dissolution in a developer and capable of maintaining the height of a small-size pattern formed therein. The surfactant (E) may be used alone or in admixture.

### (F) Other Components

The resist composition may further comprise (F) another component, for example, a compound which is decomposed with an acid to generate another acid (i.e., acid amplifier compound), an organic acid derivative, a fluorinated alcohol, and a compound having a Mw of up to 3,000 which changes its solubility in developer under the action of an acid (i.e., dissolution inhibitor). Specifically, the acid amplifier compound is described in JP-A 2009-269953 and JP-A 2010-215608 and preferably used in an amount of 0 to 5 parts, more preferably 0 to 3 parts by weight per 80 parts by weight of the base polymer (A). An extra amount of the acid amplifier compound can make the acid diffusion control difficult and cause degradations to resolution and pattern profile. With respect to the remaining additives, reference should be made to JP-A 2009-269953 and JP-A 2010-215608.

### [Process]

A further embodiment of the invention is a process of forming a pattern from the resist composition defined above by lithography. The preferred process includes the steps of applying the resist composition onto a substrate to form a resist film thereon, exposing the resist film to high-energy radiation, and developing the exposed resist film in a developer. Any desired steps may be added to the process if necessary.

The substrate used herein may be a substrate for integrated circuitry fabrication, e.g., Si, SiO₂, SiN, SiON, TiN, WSi, BPSG, SOG, organic antireflective film, etc. or a substrate for mask circuitry fabrication, e.g., Cr, CrO, CrON, MoSi₂, SiO₂, etc.

The resist composition is applied onto a substrate by a suitable coating technique such as spin coating. The coating is prebaked on a hot plate preferably at a temperature of 60 to 150° C. for 1 to 10 minutes, more preferably at 80 to 140° C. for 1 to 5 minutes. The resulting resist film preferably has a thickness of 0.05 to 2 μm.

Then the resist film is exposed to a pattern of high-energy radiation, typically KrF or ArF excimer laser, EUV of wavelength 3 to 15 nm or EB. On use of KrF excimer laser, ArF excimer laser or EUV, the resist film is exposed through a mask having a desired pattern, preferably in a dose of 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid having a refractive index of at least 1.0 between the resist film and the projection lens may be employed if desired. The liquid is typically water, and in this case, a protective film which is insoluble in water may be formed on the resist film.

While the water-insoluble protective film serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB), for example, on a hotplate preferably at 60 to 150° C. for 1 to 5 minutes, more preferably at 80 to 140° C. for 1 to 3 minutes.

The resist film is then developed with a developer in the form of an aqueous base solution, for example, 0.1 to 5 wt %, preferably 2 to 3 wt % aqueous solution of tetramethylammonium hydroxide (TMAH) for 0.1 to 3 minutes, preferably 0.5 to 2 minutes by conventional techniques such as dip, puddle and spray techniques. In this way, the exposed region of the resist film is dissolved away, and a desired resist pattern is formed on the substrate.

Any desired step may be added to the pattern forming process. For example, after the resist film is formed, a step of rinsing with pure water may be introduced to extract the acid generator or the like from the film surface or wash away particles. After exposure, a step of rinsing may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process, negative tone development may also be used. That is, an organic solvent may be used instead of the aqueous alkaline solution as the developer for developing and dissolving away the unexposed region of the resist film.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. These organic solvents may be used alone or in admixture of two or more.

### EXAMPLES

Synthesis Examples, Examples and Comparative Examples are given below by way of illustration and not by way of limitation. The abbreviation “pbw” is parts by weight. Analysis is made by IR and proton-NMR spectroscopy using the analyzers shown below.

IR: NICOLET 6700 by Thermo Fisher Scientific Inc.

¹H-NMR: ECA-500 by JEOL Ltd.

### [1] Synthesis of Monomers

### Example 1-1

### Synthesis of Monomer A-1

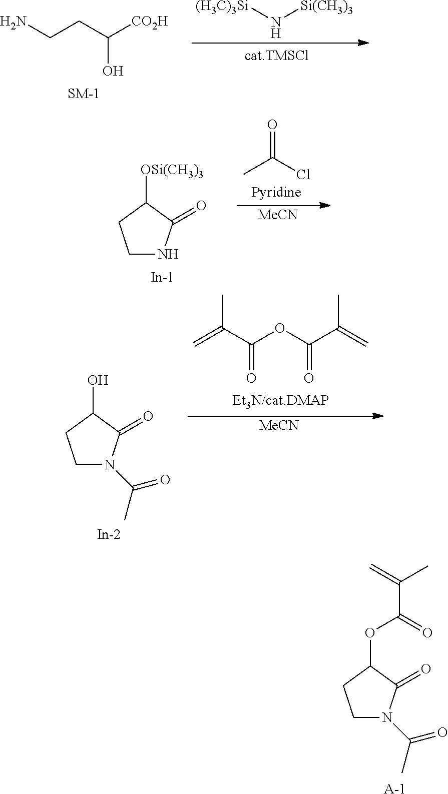

### (1) Synthesis of Intermediate In-1

Under nitrogen atmosphere, a reactor was charged with 75 g of 4-amino-2-hydroxybutanoic acid, 203 g of hexamethyldisilazane, and 600 ml of toluene. To the solution, 3.4 g of chlorotrimethylsilane was added dropwise at room temperature. After the temperature was raised, the solution was heated at an internal temperature of 120° C. under reflux for 4 hours. The reaction solution was cooled and poured into 700 ml of ethanol to quench the reaction. The solution was concentrated, the concentrate was dissolved in methylene chloride, and the insoluble was filtered off. The filtrate was concentrated, obtaining 100 g of Intermediate In-1 as colorless oily matter (crude yield 92%). Intermediate In-1 was used in the next step without further purification.

### (2) Synthesis of Intermediate In-2

Under nitrogen atmosphere, a reactor was charged with 100 g of Intermediate In-1, 100 g of pyridine, and 400 g of acetonitrile. With cooling, 79 g of acetyl chloride was added dropwise. The solution was stirred at room temperature for 12 hours, after which it was cooled again. To the solution, 100 ml of saturated sodium chloride solution and 50 ml of water were added to quench the reaction. From the water layer, the desired compound was extracted with ethyl acetate. The organic layer was dried over sodium sulfate. After the sodium sulfate was removed by filtration, the filtrate was evaporated to dryness under reduced pressure. The solids were washed with hexane. The solids were filtered and dried, obtaining 115 g of Intermediate In-2 as white crystals (yield 96%).

Intermediate In-2 was analyzed by IR and proton NMR, with the results shown below.

IR (D-ATR): ν=3401, 3007, 2972, 2915, 1718, 1705, 1694, 1480, 1461, 1416, 1370, 1318, 1298, 1280, 1242, 1197, 1128, 1091, 1045, 1004, 964, 920, 880, 763, 723, 615, 598, 559 cm⁻¹

¹H-NMR (600 MHz in DMSO-d6): δ=5.87 (1H, d), 4.31 (1H, ddd), 3.68 (1H, ddd), 3.32 (1H, ddd), 2.36 (3H, s), 2.26 (1H, dddd), 1.69 (1H, dddd) ppm

### (3) Synthesis of Monomer A-1

Under nitrogen atmosphere, a reactor was charged with 115 g of Intermediate In-2, 121 g of triethylamine, 4.9 g of 4-dimethylaminopyridine, and 400 ml of acetonitrile. At an internal temperature of 30° C., 154 g of methacrylic anhydride was added dropwise. At the end of addition, the solution was aged at an internal temperature of 45° C. for 2 hours. Thereafter, 100 ml of saturated sodium bicarbonate solution and 100 ml of water were added to quench the reaction. The target compound was extracted with 300 ml of ethyl acetate. This was followed by standard aqueous work-up, solvent distillation, and purification by distillation, obtaining 164 g of Monomer A-1 as colorless oil (3-step yield 86%).

Monomer A-1 was analyzed by IR and proton NMR, with the results shown below.

IR (D-ATR): ν=2931, 1753, 1723, 1704, 1637, 1484, 1454, 1418, 1374, 1298, 1252, 1158, 1108, 1086, 1041, 994, 948, 912, 812, 763, 717, 607, 552 cm-1

¹H-NMR (600 MHz in DMSO-d6): δ=6.10 (1H, s), 5.78 (1H, s), 5.61 (1H, dd), 3.79 (1H, dd), 3.46 (1H, ddd), 2.45 (1H, dddd), 2.37 (3H, s), 1.98 (1H, dddd), 1.90 (3H, s) ppm

### Examples 1-2 to 1-10

### Synthesis of Monomers A-2 to A-10

Monomers A-2 to A-10 shown below were similarly synthesized using the corresponding reactants and organic synthesis reaction.

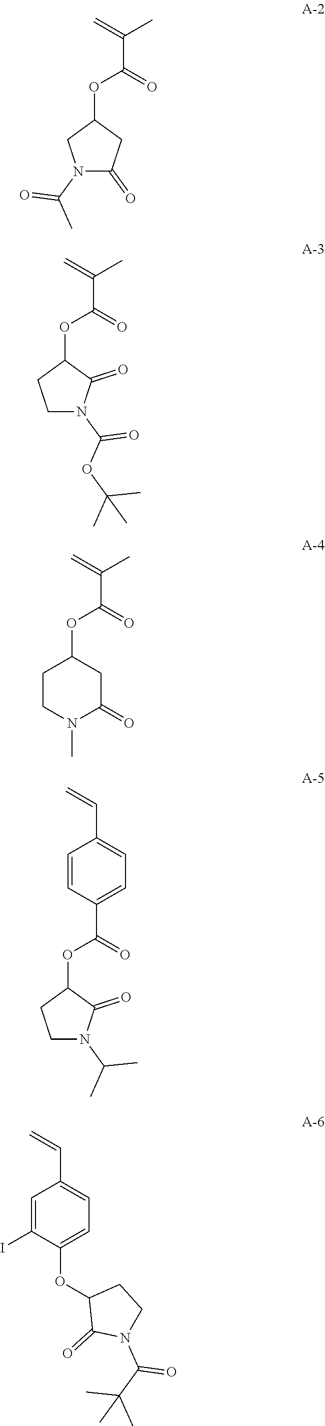

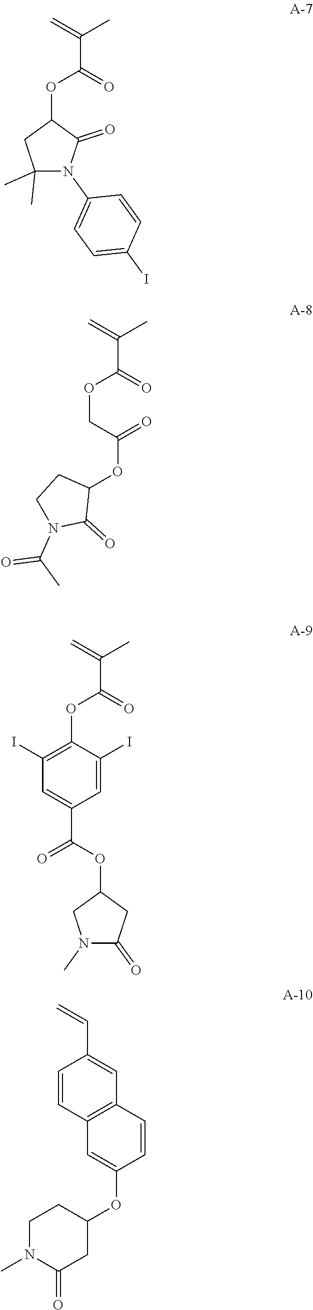

### Comparative Examples 1-1 to 1-9

Synthesis of Monomers cA-1 to cA-9

Comparative Monomers cA-1 to cA-9 were similarly synthesized using the corresponding reactants and organic synthesis reaction.

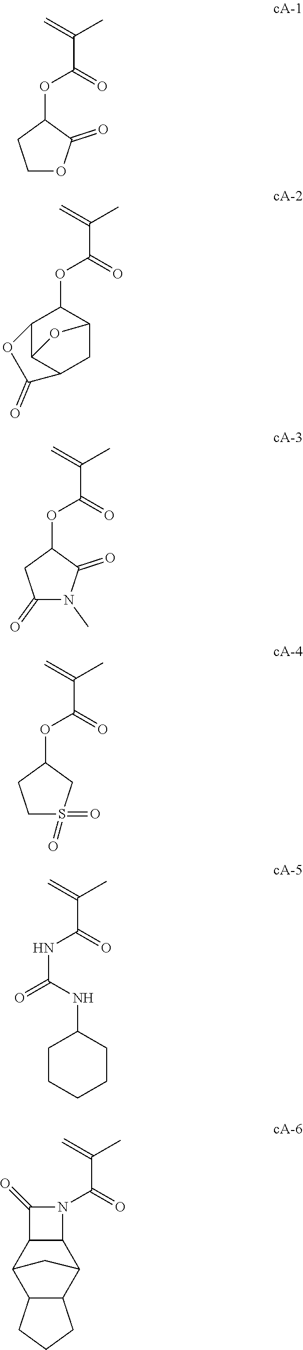

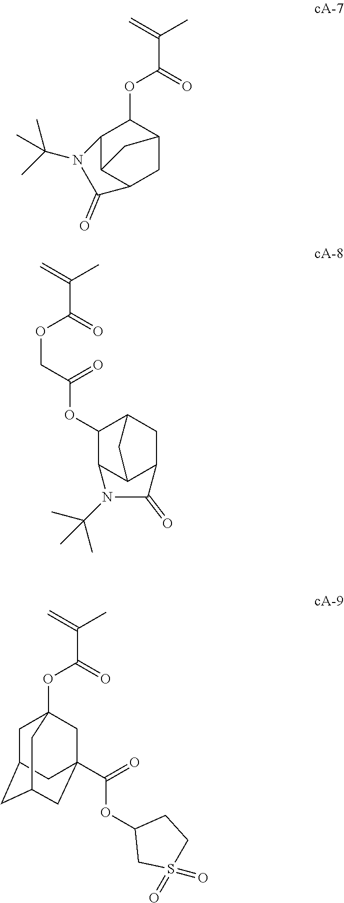

### [2] Synthesis of Base Polymers

A variety of monomers were used in the synthesis of polymers. The monomers other than Monomers A-1 to A-10 and cA-1 to cA-9 are shown below.

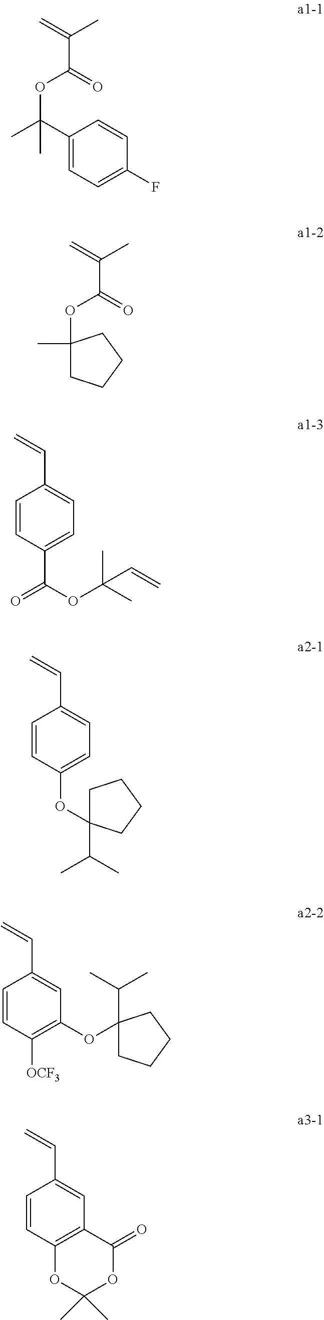 [SMILES: C=C(C)C(=O)OC(C)(C)c1ccc(F)cc1 ; C=C(C)C(=O)OC1(C)CCCC1 ; C=Cc1ccc(C(=O)OC(C)(C)C=C)cc1 ; C=Cc1ccc(OC2(C(C)C)CCCC2)cc1 ; C=Cc1ccc(OC(F)(F)F)c(OC2(C(C)C)CCCC2)c1 ; C=Cc1ccc2c(c1)C(=O)OC(C)(C)O2]

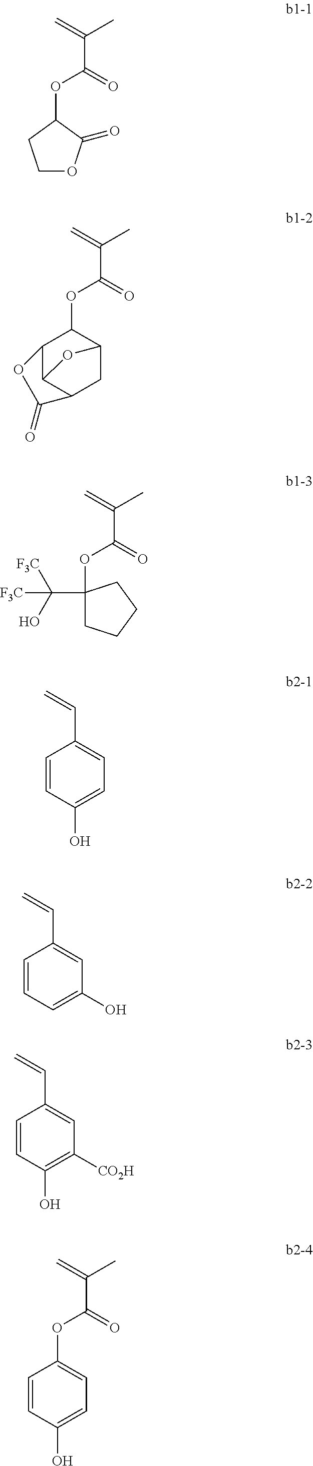

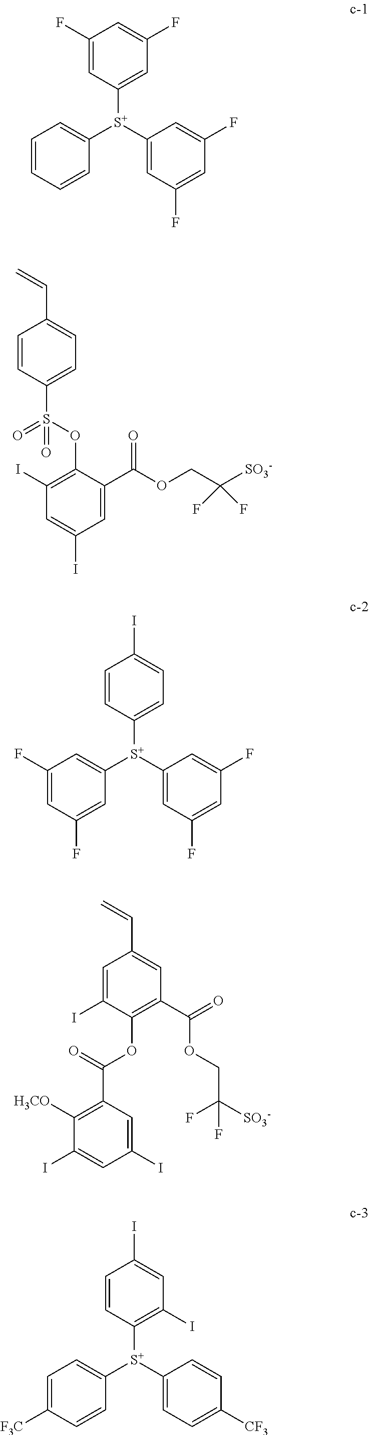

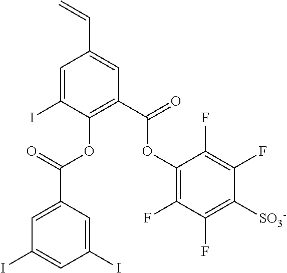

### Example 2-1

### Synthesis of Polymer P-1

A flask under nitrogen atmosphere was charged with 6.9 g of Monomer A-1, 35.2 g of Monomer a1-1, 9.5 g of Monomer b2-1, 48.9 g of Monomer c-1, 3.63 g of dimethyl 2,2′-azobis(2-methylpropionate) (trade name V601, FUJIFILM Wako Pure Chemical Corp.), and 140 g of methyl ethyl ketone (MEK) solvent to form a monomer/initiator solution. Another flask under nitrogen atmosphere was charged with 46 g of MEK, which was heated at 80° C. with stirring. The monomer/initiator solution was added dropwise to the flask over 4 hours. At the end of dropwise addition, the solution was continuously stirred for 2 hours while the temperature of 80° C. was maintained during polymerization. The solution was cooled at room temperature, after which it was added dropwise to 2,000 g of hexane with vigorous stirring. The precipitated polymer was collected by filtration, washed twice with 600 g of hexane, and vacuum dried at 50° C. for 20 hours, obtaining Polymer P-1 as white powder (amount 96.1 g, yield 96%). Polymer P-1 had a Mw of 9,700 and a Mw/Mn of 1.54. It is noted that Mw is determined by GPC versus polystyrene standards using DMF solvent.

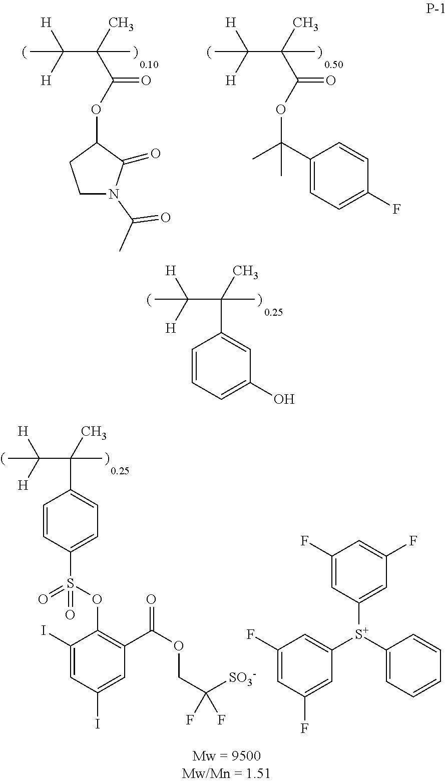

### Examples 2-2 to 2-60 and Comparative Examples 2-1 to 2-52

### Synthesis of Polymers P-2 to P-60 and Comparative Polymers CP-1 to CP-52

The polymers shown in Tables 1 to 4 were synthesized by the same procedure as in Example 2-1 except that the type and amount of monomers were changed.

TABLE 1

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | Mw/ |
| Polymer | Unit 1 | (mol %) | Unit 2 | (mol %) | Unit 3 | (mol %) | Unit 4 | (mol %) | Unit 5 | (mol %) | Mw | Mn |
| P-1 | A-1 | 10 | a1-1 | 50 | b2-1 | 25 | c-1 | 15 | — | — | 9,500 | 1.51 |
| P-2 | A-2 | 10 | a1-1 | 50 | b2-1 | 25 | c-1 | 15 | — | — | 9,300 | 1.51 |
| P-3 | A-3 | 10 | a1-1 | 50 | b2-1 | 25 | c-1 | 15 | — | — | 9,200 | 1.53 |
| P-4 | A-4 | 10 | a1-1 | 50 | b2-1 | 25 | c-1 | 15 | — | — | 9,100 | 1.52 |
| P-5 | A-5 | 10 | a1-1 | 50 | b2-1 | 25 | c-1 | 15 | — | — | 9,400 | 1.54 |
| P-6 | A-6 | 10 | a1-1 | 50 | b2-1 | 25 | c-1 | 15 | — | — | 9,500 | 1.53 |
| P-7 | A-7 | 10 | a1-1 | 50 | b2-1 | 25 | c-1 | 15 | — | — | 9,300 | 1.51 |
| P-8 | A-8 | 10 | a1-1 | 50 | b2-1 | 25 | c-1 | 15 | — | — | 9,200 | 1.52 |
| P-9 | A-9 | 10 | a1-1 | 50 | b2-1 | 25 | c-1 | 15 | — | — | 9,300 | 1.51 |
| P-10 | A-10 | 10 | a1-1 | 50 | b2-1 | 25 | c-1 | 15 | — | — | 9,200 | 1.53 |
| P-11 | A-1 | 10 | a1-1 | 50 | b2-1 | 25 | c-2 | 15 | — | — | 9,100 | 1.52 |
| P-12 | A-2 | 10 | a1-1 | 50 | b2-1 | 25 | c-2 | 15 | — | — | 9,300 | 1.55 |
| P-13 | A-3 | 10 | a1-1 | 50 | b2-1 | 25 | c-2 | 15 | — | — | 9,200 | 1.51 |
| P-14 | A-4 | 10 | a1-1 | 50 | b2-1 | 25 | c-2 | 15 | — | — | 9,400 | 1.53 |
| P-15 | A-5 | 10 | a1-1 | 50 | b2-1 | 25 | c-2 | 15 | — | — | 9,400 | 1.54 |
| P-16 | A-6 | 10 | a1-1 | 50 | b2-1 | 25 | c-2 | 15 | — | — | 9,300 | 1.52 |
| P-17 | A-7 | 10 | a1-1 | 50 | b2-1 | 25 | c-2 | 15 | — | — | 9,400 | 1.55 |
| P-18 | A-8 | 10 | a1-1 | 50 | b2-1 | 25 | c-2 | 15 | — | — | 9,100 | 1.53 |
| P-19 | A-9 | 10 | a1-1 | 50 | b2-1 | 25 | c-2 | 15 | — | — | 9,400 | 1.51 |
| P-20 | A-10 | 10 | a1-1 | 50 | b2-1 | 25 | c-2 | 15 | — | — | 9,600 | 1.56 |
| P-21 | A-1 | 10 | a1-1 | 50 | b2-1 | 25 | c-3 | 15 | — | — | 9,200 | 1.52 |
| P-22 | A-2 | 10 | a1-1 | 50 | b2-1 | 25 | c-3 | 15 | — | — | 9,500 | 1.51 |
| P-23 | A-4 | 10 | a1-1 | 50 | b2-1 | 25 | c-3 | 15 | — | — | 9,100 | 1.53 |
| P-24 | A-6 | 10 | a1-1 | 50 | b2-1 | 25 | c-3 | 15 | — | — | 9,300 | 1.52 |
| P-25 | A-9 | 10 | a1-1 | 50 | b2-1 | 25 | c-3 | 15 | — | — | 9,200 | 1.55 |
| P-26 | A-1 | 10 | a1-2 | 50 | b2-1 | 25 | c-1 | 15 | — | — | 9,500 | 1.51 |
| P-27 | A-2 | 10 | a1-2 | 50 | b2-1 | 25 | c-1 | 15 | — | — | 9,200 | 1.54 |
| P-28 | A-3 | 10 | a1-2 | 50 | b2-1 | 25 | c-1 | 15 | — | — | 9,100 | 1.52 |
| P-29 | A-5 | 10 | a1-2 | 50 | b2-1 | 25 | c-1 | 15 | — | — | 9,500 | 1.53 |
| P-30 | A-8 | 10 | a1-2 | 50 | b2-1 | 25 | c-1 | 15 | — | — | 9,200 | 1.54 |

TABLE 2

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | Mw/ |
| Polymer | Unit 1 | (mol %) | Unit 2 | (mol %) | Unit 3 | (mol %) | Unit 4 | (mol %) | Unit 5 | (mol %) | Mw | Mn |
| P-31 | A-1 | 10 | a1-3 | 50 | b2-1 | 25 | c-1 | 15 | — | — | 9,100 | 1.52 |
| P-32 | A-2 | 10 | a1-3 | 50 | b2-2 | 25 | c-2 | 15 | — | — | 9,200 | 1.54 |
| P-33 | A-6 | 10 | a1-3 | 50 | b2-3 | 25 | c-3 | 15 | — | — | 9,300 | 1.51 |
| P-34 | A-7 | 10 | a1-3 | 50 | b2-4 | 25 | c-1 | 15 | — | — | 9,500 | 1.54 |
| P-35 | A-10 | 10 | a1-3 | 50 | b2-2 | 25 | c-1 | 15 | — | — | 9,200 | 1.51 |
| P-36 | A-1 | 10 | a2-1 | 50 | b2-1 | 25 | c-1 | 15 | — | — | 9,300 | 1.52 |
| P-37 | A-2 | 10 | a2-1 | 50 | b2-2 | 25 | c-2 | 15 | — | — | 9,400 | 1.53 |
| P-38 | A-4 | 10 | a2-1 | 50 | b2-3 | 25 | c-3 | 15 | — | — | 9,200 | 1.51 |
| P-39 | A-1 | 10 | a3-1 | 45 | b2-4 | 25 | c-1 | 15 | — | — | 9,100 | 1.53 |
| P-40 | A-1 | 10 | a1-1 | 30 | a1-2 | 25 | b2-1 | 25 | c-1 | 10 | 9,300 | 1.55 |
| P-41 | A-1 | 10 | a1-1 | 30 | a1-3 | 25 | b2-1 | 25 | c-1 | 10 | 9,500 | 1.52 |
| P-42 | A-1 | 10 | a1-1 | 30 | a2-1 | 25 | b2-2 | 25 | c-1 | 10 | 9,100 | 1.52 |
| P-43 | A-2 | 15 | a1-1 | 30 | a3-1 | 15 | b2-1 | 30 | c-2 | 10 | 9,300 | 1.52 |
| P-44 | A-1 | 10 | a1-3 | 45 | b1-1 | 5 | b2-1 | 30 | c-1 | 10 | 9,200 | 1.53 |
| P-45 | A-2 | 10 | a1-1 | 45 | b1-2 | 5 | b2-1 | 25 | c-1 | 15 | 9,100 | 1.54 |
| P-46 | A-1 | 15 | a1-1 | 50 | b2-1 | 35 | — | — | — | — | 5,500 | 1.53 |
| P-47 | A-2 | 15 | a1-1 | 50 | b2-1 | 35 | — | — | — | — | 5,400 | 1.52 |
| P-48 | A-5 | 15 | a1-1 | 50 | b2-1 | 35 | — | — | — | — | 5,700 | 1.54 |
| P-49 | A-8 | 15 | a1-1 | 50 | b2-1 | 35 | — | — | — | — | 5,600 | 1.53 |
| P-50 | A-9 | 15 | a1-1 | 50 | b2-1 | 35 | — | — | — | — | 6,000 | 1.51 |
| P-51 | A-1 | 15 | a1-1 | 50 | b2-2 | 35 | — | — | — | — | 6,200 | 1.52 |
| P-52 | A-2 | 15 | a1-1 | 50 | b2-2 | 35 | — | — | — | — | 5,500 | 1.51 |
| P-53 | A-1 | 15 | a1-2 | 50 | b2-2 | 35 | — | — | — | — | 5,600 | 1.53 |
| P-54 | A-4 | 20 | a1-3 | 50 | b2-3 | 30 | — | — | — | — | 6,200 | 1.52 |
| P-55 | A-1 | 15 | a1-1 | 25 | a2-1 | 25 | b2-1 | 30 | b1-1 | 5 | 5,400 | 1.54 |
| P-56 | A-1 | 15 | a1-1 | 25 | a2-1 | 25 | b2-1 | 30 | b1-2 | 5 | 5,800 | 1.53 |
| P-57 | A-1 | 10 | a1-1 | 50 | b2-2 | 30 | b1-2 | 10 | — | — | 5,700 | 1.53 |
| P-58 | A-4 | 20 | a1-1 | 50 | b2-3 | 30 | — | — | — | — | 6,000 | 1.54 |
| P-59 | A-2 | 5 | a3-1 | 30 | b2-2 | 65 | — | — | — | — | 7,100 | 1.53 |
| P-60 | A-1 | 10 | cA-1 | 10 | a1-1 | 50 | a2-1 | 30 | — | — | 6,200 | 1.55 |

TABLE 3

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | Mw/ |
| Polymer | Unit 1 | (mol %) | Unit 2 | (mol %) | Unit 3 | (mol %) | Unit 4 | (mol %) | Unit 5 | (mol %) | Mw | Mn |
| CP-1 | cA-1 | 10 | a1-1 | 50 | b2-1 | 25 | c-1 | 15 | — | — | 9,500 | 1.51 |
| CP-2 | cA-2 | 10 | a1-1 | 50 | b2-1 | 25 | c-1 | 15 | — | — | 9,300 | 1.51 |
| CP-3 | cA-3 | 10 | a1-1 | 50 | b2-1 | 25 | c-1 | 15 | — | — | 9,200 | 1.53 |
| CP-4 | cA-4 | 10 | a1-1 | 50 | b2-1 | 25 | c-1 | 15 | — | — | 9,100 | 1.52 |
| CP-5 | cA-5 | 10 | a1-1 | 50 | b2-1 | 25 | c-1 | 15 | — | — | 9,400 | 1.54 |
| CP-6 | cA-6 | 10 | a1-1 | 50 | b2-1 | 25 | c-1 | 15 | — | — | 9,500 | 1.53 |
| CP-7 | cA-7 | 10 | a1-1 | 50 | b2-1 | 25 | c-1 | 15 | — | — | 9,300 | 1.51 |
| CP-8 | cA-8 | 10 | a1-1 | 50 | b2-1 | 25 | c-1 | 15 | — | — | 9,200 | 1.52 |
| CP-9 | cA-9 | 10 | a1-1 | 50 | b2-1 | 25 | c-1 | 15 | — | — | 9,500 | 1.53 |
| CP-10 | cA-1 | 10 | a1-1 | 50 | b2-1 | 25 | c-2 | 15 | — | — | 9,300 | 1.51 |
| CP-11 | cA-2 | 10 | a1-1 | 50 | b2-1 | 25 | c-2 | 15 | — | — | 9,200 | 1.53 |
| CP-12 | cA-3 | 10 | a1-1 | 50 | b2-1 | 25 | c-2 | 15 | — | — | 9,300 | 1.55 |
| CP-13 | cA-4 | 10 | a1-1 | 50 | b2-1 | 25 | c-2 | 15 | — | — | 9,200 | 1.52 |
| CP-14 | cA-5 | 10 | a1-1 | 50 | b2-1 | 25 | c-2 | 15 | — | — | 9,100 | 1.52 |
| CP-15 | cA-6 | 10 | a1-1 | 50 | b2-1 | 25 | c-2 | 15 | — | — | 9,300 | 1.52 |
| CP-16 | cA-7 | 10 | a1-1 | 50 | b2-1 | 25 | c-2 | 15 | — | — | 9,200 | 1.53 |
| CP-17 | cA-8 | 10 | a1-1 | 50 | b2-1 | 25 | c-2 | 15 | — | — | 9,300 | 1.54 |
| CP-18 | cA-9 | 10 | a1-1 | 50 | b2-1 | 25 | c-2 | 15 | — | — | 9,300 | 1.53 |
| CP-19 | cA-1 | 10 | a1-1 | 50 | b2-1 | 25 | c-3 | 15 | — | — | 9,200 | 1.52 |
| CP-20 | cA-4 | 10 | a1-1 | 50 | b2-1 | 25 | c-3 | 15 | — | — | 9,500 | 1.54 |
| CP-21 | cA-7 | 10 | a1-1 | 50 | b2-1 | 25 | c-3 | 15 | — | — | 9,100 | 1.53 |
| CP-22 | cA-6 | 10 | a1-2 | 50 | b2-1 | 25 | c-1 | 15 | — | — | 9,300 | 1.51 |
| CP-23 | cA-8 | 10 | a1-2 | 50 | b2-1 | 25 | c-1 | 15 | — | — | 9,200 | 1.52 |
| CP-24 | cA-1 | 10 | a1-3 | 50 | b2-1 | 25 | c-1 | 15 | — | — | 9,500 | 1.51 |
| CP-25 | cA-3 | 10 | a1-3 | 50 | b2-2 | 25 | c-2 | 15 | — | — | 9,200 | 1.53 |
| CP-26 | cA-5 | 10 | a1-3 | 50 | b2-3 | 25 | c-3 | 15 | — | — | 9,100 | 1.52 |
| CP-27 | cA-7 | 10 | a1-3 | 50 | b2-4 | 25 | c-1 | 15 | — | — | 9,500 | 1.54 |
| CP-28 | cA-1 | 10 | a2-1 | 50 | b2-1 | 25 | c-1 | 15 | — | — | 9,200 | 1.53 |
| CP-29 | cA-3 | 10 | a2-1 | 50 | b2-2 | 25 | c-2 | 15 | — | — | 9,500 | 1.53 |
| CP-30 | cA-4 | 10 | a2-1 | 50 | b2-3 | 25 | c-3 | 15 | — | — | 9,300 | 1.54 |

TABLE 4

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | Mw/ |
| Polymer | Unit 1 | (mol %) | Unit 2 | (mol %) | Unit 3 | (mol %) | Unit 4 | (mol %) | Unit 5 | (mol %) | Mw | Mn |
| CP-31 | cA-2 | 10 | a3-1 | 45 | b2-4 | 25 | c-1 | 15 | — | — | 9,200 | 1.53 |
| CP-32 | cA-3 | 10 | a1-1 | 30 | a1-2 | 25 | b2-1 | 25 | c-1 | 10 | 9,100 | 1.51 |
| CP-33 | cA-4 | 10 | a1-1 | 30 | a1-3 | 25 | b2-1 | 25 | c-1 | 10 | 9,400 | 1.52 |
| CP-34 | cA-1 | 10 | a1-1 | 30 | a2-1 | 25 | b2-2 | 25 | c-1 | 10 | 9,500 | 1.53 |
| CP-35 | cA-2 | 15 | a1-1 | 30 | a3-1 | 15 | b2-1 | 30 | c-2 | 10 | 9,300 | 1.53 |
| CP-36 | cA-3 | 10 | a1-3 | 45 | b1-1 | 5 | b2-1 | 30 | c-1 | 10 | 9,200 | 1.54 |
| CP-37 | cA-7 | 10 | a1-1 | 45 | b1-2 | 5 | b2-1 | 25 | c-1 | 15 | 9,300 | 1.53 |
| CP-38 | cA-1 | 15 | a1-1 | 50 | b2-1 | 35 | — | — | — | — | 5,500 | 1.53 |
| CP-39 | cA-2 | 15 | a1-1 | 50 | b2-1 | 35 | — | — | — | — | 5,400 | 1.52 |
| CP-40 | cA-7 | 15 | a1-1 | 50 | b2-1 | 35 | — | — | — | — | 5,700 | 1.54 |
| CP-41 | cA-8 | 15 | a1-1 | 50 | b2-1 | 35 | — | — | — | — | 5,600 | 1.53 |
| CP-42 | cA-9 | 15 | a1-1 | 50 | b2-1 | 35 | — | — | — | — | 6,000 | 1.51 |
| CP-43 | cA-1 | 15 | a1-1 | 50 | b2-2 | 35 | — | — | — | — | 6,200 | 1.52 |
| CP-44 | cA-2 | 15 | a1-1 | 50 | b2-2 | 35 | — | — | — | — | 5,500 | 1.51 |
| CP-45 | cA-3 | 15 | a1-2 | 50 | b2-2 | 35 | — | — | — | — | 5,600 | 1.53 |
| CP-46 | cA-4 | 20 | a1-3 | 50 | b2-3 | 30 | — | — | — | — | 6,200 | 1.52 |
| CP-47 | cA-1 | 15 | a1-1 | 25 | a2-1 | 25 | b2-1 | 30 | b1-1 | 5 | 5,400 | 1.54 |
| CP-48 | cA-6 | 15 | a1-1 | 25 | a2-1 | 25 | b2-1 | 30 | b1-2 | 5 | 5,800 | 1.53 |
| CP-49 | cA-7 | 10 | a1-1 | 50 | b2-2 | 30 | b1-2 | 10 | — | — | 5,700 | 1.53 |
| CP-50 | cA-3 | 20 | a1-1 | 50 | b2-3 | 30 | — | — | — | — | 6,000 | 1.54 |
| CP-51 | cA-4 | 5 | a3-1 | 30 | b2-2 | 55 | — | — | — | — | 7,100 | 1.53 |
| CP-52 | cA-1 | 10 | cA-3 | 10 | a1-1 | 50 | a2-1 | 30 | — | — | 6,200 | 1.55 |

### [3] Preparation of Chemically Amplified Resist Compositions

### Examples 3-1 to 3-60 and Comparative Examples 3-1 to 3-52

A chemically amplified resist composition (R-1 to R-60, CR-1 to CR-52) in solution form was prepared by dissolving a base polymer (P-1 to P-60), comparative base polymer (CP-1 to CP-52), photoacid generator (PAG-X and PAG-Y), and quencher (Q-1 to Q-4) in an organic solvent in accordance with the recipe shown in Tables 5 to 8, and filtering the solution through a Teflon® filter with a pore size of 0.2 μm. The organic solvent contained 0.0% by weight of surfactant A.

TABLE 5

| | | Resist | Base | | Photoacid | | | |
|---|---|---|---|---|---|---|---|---|
| | | compo- | polymer | Quencher | generator | Solvent 1 | Solvent 2 | Solvent 3 |
| | | sition | (pbw) | (pbw) | (pbw) | (pbw) | (pbw) | (pbw) |
| Example | 3-1 | R-1 | P-1 (80) | Q-1 (8.0) | — | PGMEA (2250) | EL (2800) | DAA (550) |
| | 3-2 | R-2 | P-2 (80) | Q-1 (8.2) | — | PGMEA (2250) | EL (2800) | DAA (550) |
| | 3-3 | R-3 | P-3 (80) | Q-1 (8.0) | — | PGMEA (2250) | EL (2800) | DAA (550) |
| | 3-4 | R-4 | P-4 (80) | Q-1 (7.8) | — | PGMEA (2250) | EL (2800) | DAA (550) |
| | 3-5 | R-5 | P-5 (80) | Q-1 (8.0) | — | PGMEA (2250) | EL (2800) | DAA (550) |
| | 3-6 | R-6 | P-6 (80) | Q-1 (8.0) | — | PGMEA (2250) | EL (2800) | DAA (550) |
| | 3-7 | R-7 | P-7 (80) | Q-1 (8.2) | — | PGMEA (2250) | EL (2800) | DAA (550) |
| | 3-8 | R-8 | P-8 (80) | Q-1 (8.0) | — | PGMEA (2250) | EL (2800) | DAA (550) |
| | 3-9 | R-9 | P-9 (80) | Q-1 (7.6) | — | PGMEA (2250) | EL (2800) | DAA (550) |
| | 3-10 | R-10 | P-10 (80) | Q-1 (7.6) | — | PGMEA (2250) | EL (2800) | DAA (550) |
| | 3-11 | R-11 | P-11 (80) | Q-1 (8.0) | — | PGMEA (2250) | EL (2800) | DAA (550) |
| | 3-12 | R-12 | P-12 (80) | Q-1 (8.2) | — | PGMEA (2250) | EL (2800) | DAA (550) |
| | 3-13 | R-13 | P-13 (80) | Q-1 (8.0) | — | PGMEA (2250) | EL (2800) | DAA (550) |
| | 3-14 | R-14 | P-14 (80) | Q-1 (7.8) | — | PGMEA (2250) | EL (2800) | DAA (550) |
| | 3-15 | R-15 | P-15 (80) | Q-1 (8.0) | — | PGMEA (2250) | EL (2800) | DAA (550) |
| | 3-16 | R-16 | P-16 (80) | Q-1 (8.0) | — | PGMEA (2250) | EL (2800) | DAA (550) |
| | 3-17 | R-17 | P-17 (80) | Q-1 (8.0) | — | PGMEA (2250) | EL (2800) | DAA (550) |
| | 3-18 | R-18 | P-18 (80) | Q-1 (8.0) | — | PGMEA (2250) | EL (2800) | DAA (550) |
| | 3-19 | R-19 | P-19 (80) | Q-1 (8.2) | — | PGMEA (2250) | EL (2800) | DAA (550) |
| | 3-20 | R-20 | P-20 (80) | Q-1 (8.0) | — | PGMEA (2250) | EL (2800) | DAA (550) |
| | 3-21 | R-21 | P-21 (80) | Q-1 (7.8) | — | PGMEA (2250) | EL (2800) | DAA (550) |
| | 3-22 | R-22 | P-22 (80) | Q-1 (8.0) | — | PGMEA (2250) | EL (2800) | DAA (550) |
| | 3-23 | R-23 | P-23 (80) | Q-1 (8.0) | — | PGMEA (2250) | EL (2800) | DAA (550) |
| | 3-24 | R-24 | P-24 (80) | Q-1 (8.0) | — | PGMEA (2250) | EL (2800) | DAA (550) |
| | 3-25 | R-25 | P-25 (80) | Q-1 (7.8) | — | PGMEA (2250) | EL (2800) | DAA (550) |
| | 3-26 | R-26 | P-26 (80) | Q-3 (8.0) | — | PGMEA (2250) | EL (2800) | DAA (550) |
| | 3-27 | R-27 | P-27 (80) | Q-1 (7.8) | — | PGMEA (2250) | EL (2800) | DAA (550) |
| | 3-28 | R-28 | P-28 (80) | Q-2 (8.0) | — | PGMEA (2250) | EL (2800) | DAA (550) |
| | 3-29 | R-29 | P-29 (80) | Q-3 (7.8) | — | PGMEA (2250) | EL (2800) | DAA (550) |
| | 3-30 | R-30 | P-30 (80) | Q-1 (8.0) | — | PGMEA (2250) | EL (2800) | DAA (550) |

TABLE 6

| | | Resist | Base | | Photoacid | | | |
|---|---|---|---|---|---|---|---|---|
| | | compo- | polymer | Quencher | generator | Solvent 1 | Solvent 2 | Solvent 3 |
| | | sition | (pbw) | (pbw) | (pbw) | (pbw) | (pbw) | (pbw) |
| Example | 3-31 | R-31 | P-31 (80) | Q-2 (8.2) | — | PGMEA (2250) | EL (2800) | DAA (550) |
| | 3-32 | R-32 | P-32 (80) | Q-1 (8.0) | — | PGMEA (2250) | EL (2800) | DAA (550) |
| | 3-33 | R-33 | P-33 (80) | Q-3 (7.6) | — | PGMEA (2250) | EL (2800) | DAA (550) |
| | 3-34 | R-34 | P-34 (80) | Q-1 (8.0) | — | PGMEA (2250) | EL (2800) | DAA (550) |
| | 3-35 | R-35 | P-35 (80) | Q-2 (8.2) | — | PGMEA (2250) | EL (2800) | DAA (550) |
| | 3-36 | R-36 | P-36 (80) | Q-3 (8.0) | — | PGMEA (2250) | EL (2800) | DAA (550) |
| | 3-37 | R-37 | P-37 (80) | Q-2 (8.0) | — | PGMEA (2250) | EL (2800) | DAA (550) |
| | 3-38 | R-38 | P-38 (80) | Q-1 (8.0) | — | PGMEA (2250) | EL (2800) | DAA (550) |
| | 3-39 | R-39 | P-39 (80) | Q-1 (7.8) | — | PGMEA (2250) | EL (2800) | DAA (550) |
| | 3-40 | R-40 | P-40 (80) | Q-2 (8.0) | PAG-X (10) | PGMEA (2250) | EL (2800) | DAA (550) |
| | 3-41 | R-41 | P-41 (80) | Q-3 (7.8) | PAG-Y (15) | PGMEA (2250) | EL (2800) | DAA (550) |
| | 3-42 | R-42 | P-42 (80) | Q-1 (4.0) | PAG-X (10) | PGMEA (2250) | EL (2800) | DAA (550) |
| | | | | Q-4 (3.8) | | | | |
| | 3-43 | R-43 | P-43 (80) | Q-2 (8.0) | PAG-Y (15) | PGMEA (2250) | EL (2800) | DAA (550) |
| | 3-44 | R-44 | P-44 (80) | Q-3 (7.8) | PAG-X (10) | PGMEA (2250) | EL (2800) | DAA (550) |
| | 3-45 | R-45 | P-45 (80) | Q-1 (7.8) | PAG-Y (15) | PGMEA (2250) | EL (2800) | DAA (550) |
| | 3-46 | R-46 | P-46 (80) | Q-1 (8.0) | PAG-X (24) | PGMEA (2250) | EL (2800) | DAA (550) |
| | 3-47 | R-47 | P-47 (80) | Q-1 (8.0) | PAG-X (24) | PGMEA (2250) | EL (2800) | DAA (550) |
| | 3-48 | R-48 | P-48 (80) | Q-2 (8.2) | PAG-X (24) | PGMEA (2250) | EL (2800) | DAA (550) |
| | 3-49 | R-49 | P-49 (80) | Q-2 (4.0) | PAG-Y (22) | PGMEA (2250) | EL (2800) | DAA (550) |
| | | | | Q-4 (3.8) | | | | |
| | 3-50 | R-50 | P-50 (80) | Q-3 (7.6) | PAG-X (24) | PGMEA (2250) | EL (2800) | DAA (550) |
| | 3-51 | R-51 | P-51 (80) | Q-1 (8.0) | PAG-X (22) | PGMEA (2250) | EL (2800) | DAA (550) |
| | 3-52 | R-52 | P-52 (80) | Q-3 (7.6) | PAG-Y (24) | PGMEA (2250) | EL (2800) | DAA (550) |
| | 3-53 | R-53 | P-53 (80) | Q-1 (8.0) | PAG-X (24) | PGMEA (2250) | EL (2800) | DAA (550) |
| | 3-54 | R-54 | P-54 (80) | Q-3 (7.6) | PAG-X (22) | PGMEA (2250) | EL (2800) | DAA (550) |
| | 3-55 | R-55 | P-55 (80) | Q-3 (4.0) | PAG-Y (24) | PGMEA (2250) | EL (2800) | DAA (550) |
| | | | | Q-4 (3.8) | | | | |
| | 3-56 | R-56 | P-56 (80) | Q-1 (8.0) | PAG-X (24) | PGMEA (2250) | EL (2800) | DAA (550) |
| | 3-57 | R-57 | P-57 (80) | Q-2 (8.2) | PAG-Y (24) | PGMEA (2250) | EL (2800) | DAA (550) |
| | 3-58 | R-58 | P-58 (80) | Q-3 (7.6) | PAG-Y (24) | PGMEA (2250) | EL (2800) | DAA (550) |
| | 3-59 | R-59 | P-59 (80) | Q-1 (8.0) | PAG-X (22) | PGMEA (2250) | EL (2800) | DAA (550) |
| | 3-60 | R-60 | P-60 (80) | Q-3 (7.6) | PAG-Y (24) | PGMEA (2250) | EL (2800) | DAA (550) |

TABLE 7

| | | Resist | Base | | Photoacid | | | |
|---|---|---|---|---|---|---|---|---|
| | | compo- | polymer | Quencher | generator | Solvent 1 | Solvent 2 | Solvent 3 |
| | | sition | (pbw) | (pbw) | (pbw) | (pbw) | (pbw) | (pbw) |
| Compar- | 3-1 | CR-1 | P-1 (80) | Q-1 (8.0) | — | PGMEA (2250) | EL (2800) | DAA (550) |
| ative | 3-2 | CR-2 | P-2 (80) | Q-1 (8.2) | — | PGMEA (2250) | EL (2800) | DAA (550) |
| Example | 3-3 | CR-3 | P-3 (80) | Q-1 (8.0) | — | PGMEA (2250) | EL (2800) | DAA (550) |
| | 3-4 | CR-4 | P-4 (80) | Q-1 (7.8) | — | PGMEA (2250) | EL (2800) | DAA (550) |
| | 3-5 | CR-5 | P-5 (80) | Q-1 (8.0) | — | PGMEA (2250) | EL (2800) | DAA (550) |
| | 3-6 | CR-6 | P-6 (80) | Q-1 (8.0) | — | PGMEA (2250) | EL (2800) | DAA (550) |
| | 3-7 | CR-7 | P-7 (80) | Q-1 (7.6) | — | PGMEA (2250) | EL (2800) | DAA (550) |
| | 3-8 | CR-8 | P-8 (80) | Q-1 (8.0) | — | PGMEA (2250) | EL (2800) | DAA (550) |
| | 3-9 | CR-9 | P-9 (80) | Q-1 (7.8) | — | PGMEA (2250) | EL (2800) | DAA (550) |
| | 3-10 | CR-10 | P-10 (80) | Q-1 (8.0) | — | PGMEA (2250) | EL (2800) | DAA (550) |
| | 3-11 | CR-11 | P-11 (80) | Q-1 (8.2) | — | PGMEA (2250) | EL (2800) | DAA (550) |
| | 3-12 | CR-12 | P-12 (80) | Q-1 (8.0) | — | PGMEA (2250) | EL (2800) | DAA (550) |
| | 3-13 | CR-13 | P-13 (80) | Q-1 (8.2) | — | PGMEA (2250) | EL (2800) | DAA (550) |
| | 3-14 | CR-14 | P-14 (80) | Q-1 (7.8) | — | PGMEA (2250) | EL (2800) | DAA (550) |
| | 3-15 | CR-15 | P-15 (80) | Q-1 (8.0) | — | PGMEA (2250) | EL (2800) | DAA (550) |
| | 3-16 | CR-16 | P-16 (80) | Q-1 (7.8) | — | PGMEA (2250) | EL (2800) | DAA (550) |
| | 3-17 | CR-17 | P-17 (80) | Q-1 (7.8) | — | PGMEA (2250) | EL (2800) | DAA (550) |
| | 3-18 | CR-18 | P-18 (80) | Q-1 (8.2) | — | PGMEA (2250) | EL (2800) | DAA (550) |
| | 3-19 | CR-19 | P-19 (80) | Q-1 (8.2) | — | PGMEA (2250) | EL (2800) | DAA (550) |
| | 3-20 | CR-20 | P-20 (80) | Q-1 (8.2) | — | PGMEA (2250) | EL (2800) | DAA (550) |
| | 3-21 | CR-21 | P-21 (80) | Q-1 (7.8) | — | PGMEA (2250) | EL (2800) | DAA (550) |
| | 3-22 | CR-22 | P-22 (80) | Q-1 (8.0) | — | PGMEA (2250) | EL (2800) | DAA (550) |
| | 3-23 | CR-23 | P-23 (80) | Q-1 (8.0) | — | PGMEA (2250) | EL (2800) | DAA (550) |
| | 3-24 | CR-24 | P-24 (80) | Q-1 (8.0) | — | PGMEA (2250) | EL (2800) | DAA (550) |
| | 3-25 | CR-25 | P-25 (80) | Q-1 (7.8) | — | PGMEA (2250) | EL (2800) | DAA (550) |
| | 3-26 | CR-26 | P-26 (80) | Q-3 (8.0) | — | PGMEA (2250) | EL (2800) | DAA (550) |
| | 3-27 | CR-27 | P-27 (80) | Q-1 (7.8) | — | PGMEA (2250) | EL (2800) | DAA (550) |
| | 3-28 | CR-28 | P-28 (80) | Q-2 (8.0) | — | PGMEA (2250) | EL (2800) | DAA (550) |
| | 3-29 | CR-29 | P-29 (80) | Q-3 (7.8) | — | PGMEA (2250) | EL (2800) | DAA (550) |
| | 3-30 | CR-30 | P-30 (80) | Q-1 (8.0) | — | PGMEA (2250) | EL (2800) | DAA (550) |

TABLE 8

| | | Resist | Base | | Photoacid | | | |
|---|---|---|---|---|---|---|---|---|
| | | compo- | polymer | Quencher | generator | Solvent 1 | Solvent 2 | Solvent 3 |
| | | sition | (pbw) | (pbw) | (pbw) | (pbw) | (pbw) | (pbw) |
| Compar- | 3-31 | CR-31 | P-31 (80) | Q-2 (8.2) | — | PGMEA (2250) | EL (2800) | DAA (550) |
| ative | 3-32 | CR-32 | P-32 (80) | Q-2 (8.0) | PAG-X (10) | PGMEA (2250) | EL (2800) | DAA (550) |
| Example | 3-33 | CR-33 | P-33 (80) | Q-3 (7.8) | PAG-Y (15) | PGMEA (2250) | EL (2800) | DAA (550) |
| | 3-34 | CR-34 | P-34 (80) | Q-1 (4.0) | PAG-X (10) | PGMEA (2250) | EL (2800) | DAA (550) |
| | | | | Q-4 (3.8) | | | | |
| | 3-35 | CR-35 | P-35 (80) | Q-2 (8.0) | PAG-Y (15) | PGMEA (2250) | EL (2800) | DAA (550) |
| | 3-36 | CR-36 | P-36 (80) | Q-3 (7.8) | PAG-X (10) | PGMEA (2250) | EL (2800) | DAA (550) |
| | 3-37 | CR-37 | P-37 (80) | Q-1 (7.8) | PAG-Y (15) | PGMEA (2250) | EL (2800) | DAA (550) |
| | 3-38 | CR-38 | P-38 (80) | Q-1 (8.0) | PAG-X (24) | PGMEA (2250) | EL (2800) | DAA (550) |
| | 3-39 | CR-39 | P-39 (80) | Q-1 (8.0) | PAG-X (24) | PGMEA (2250) | EL (2800) | DAA (550) |
| | 3-40 | CR-40 | P-40 (80) | Q-2 (8.2) | PAG-X (24) | PGMEA (2250) | EL (2800) | DAA (550) |
| | 3-41 | CR-41 | P-41 (80) | Q-2 (4.0) | PAG-Y (22) | PGMEA (2250) | EL (2800) | DAA (550) |
| | | | | Q-4 (3.8) | | | | |
| | 3-42 | CR-42 | P-42 (80) | Q-3 (7.6) | PAG-X (24) | PGMEA (2250) | EL (2800) | DAA (550) |
| | 3-43 | CR-43 | P-43 (80) | Q-1 (8.0) | PAG-X (22) | PGMEA (2250) | EL (2800) | DAA (550) |
| | 3-44 | CR-44 | P-44 (80) | Q-3 (7.6) | PAG-Y (24) | PGMEA (2250) | EL (2800) | DAA (550) |
| | 3-45 | CR-45 | P-45 (80) | Q-1 (8.0) | PAG-X (24) | PGMEA (2250) | EL (2800) | DAA (550) |
| | 3-46 | CR-46 | P-46 (80) | Q-3 (7.6) | PAG-X (22) | PGMEA (2250) | EL (2800) | DAA (550) |
| | 3-47 | CR-47 | P-47 (80) | Q-2 (4.0) | PAG-Y (24) | PGMEA (2250) | EL (2800) | DAA (550) |
| | | | | Q-4 (3.8) | | | | |
| | 3-48 | CR-48 | P-48 (80) | Q-1 (8.0) | PAG-X (24) | PGMEA (2250) | EL (2800) | DAA (550) |
| | 3-49 | CR-49 | P-49 (80) | Q-2 (8.2) | PAG-Y (24) | PGMEA (2250) | EL (2800) | DAA (550) |
| | 3-50 | CR-50 | P-50 (80) | Q-3 (7.6) | PAG-Y (24) | PGMEA (2250) | EL (2800) | DAA (550) |
| | 3-51 | CR-51 | P-51 (80) | Q-1 (8.0) | PAG-X (22) | PGMEA (2250) | EL (2800) | DAA (550) |
| | 3-52 | CR-52 | P-52 (80) | Q-3 (7.6) | PAG-Y (24) | PGMEA (2250) | EL (2800) | DAA (550) |

The components in Tables 5 to 8 are identified below.

### Solvent:

PGMEA (propylene glycol monomethyl ether acetate)
EL (ethyl lactate)
DAA (diacetone alcohol)
   Photoacid generators: PAG-X and PAG-Y

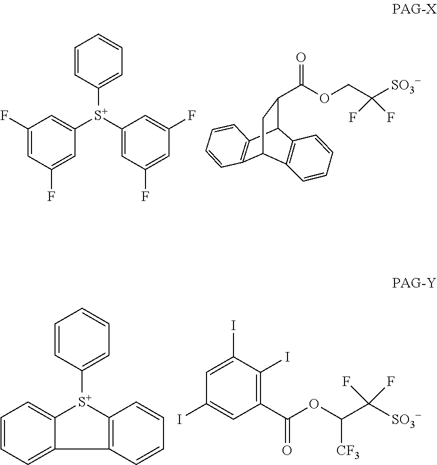

### Quenchers: Q-1 to Q-4

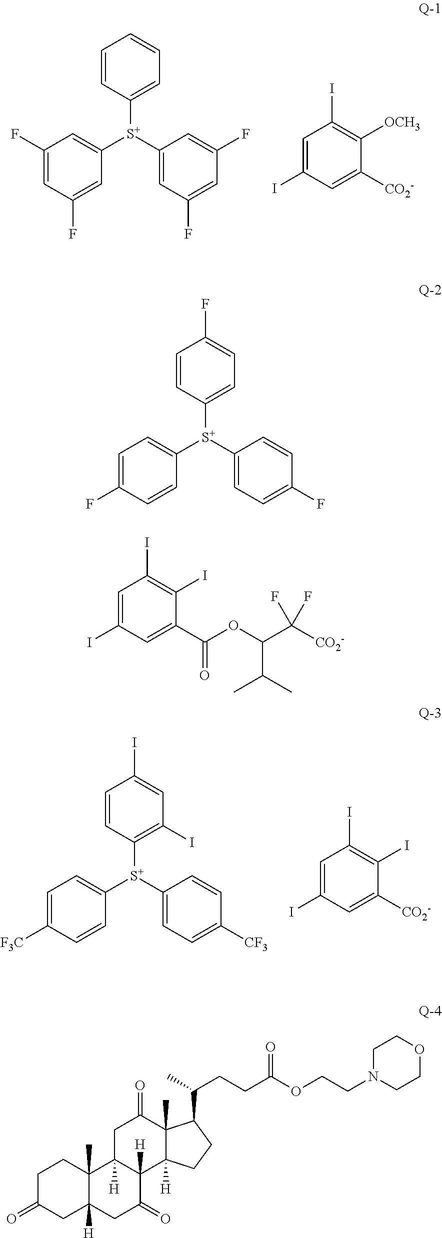

### Surfactant A:

3-methyl-3-(2,2,2-trifluoroethoxymethyl)oxetane/tetrahydrofuran/2,2-dimethyl-1,3-propanediol copolymer of the formula below (Omnova Solutions, Inc.)

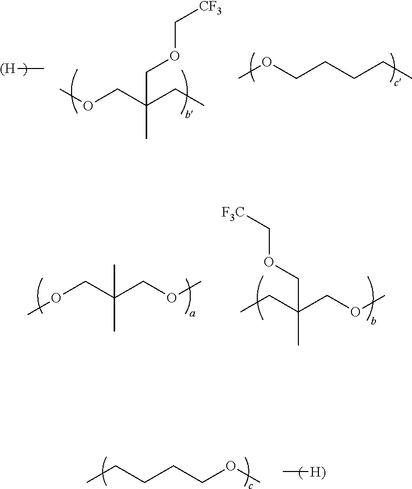

a:(b+b′):(c+c′)=1:4-7:0.01-1 (molar ratio)
Mw=1,500

### [4] EUV Lithography Test 1

### Examples 4-1 to 4-60 and Comparative Examples 4-1 to 4-52

Each of the chemically amplified resist compositions (R-1 to R-60, CR-1 to CR-52) in Tables 5 to 8 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3400 (ASML, NA 0.33, σ 0.9/0.6, dipole illumination), the resist film was exposed to EUV through a mask bearing a LS pattern having a size of 18 nm and a pitch of 36 nm (on-wafer size) while varying the dose and focus (dose pitch: 1 mJ/cm², focus pitch: 0.020 μm). The resist film was baked (PEB) on a hotplate at the temperature shown in Tables 9 to 12 for 60 seconds and puddle developed in a 2.38 wt % TMAH aqueous solution for 30 seconds, rinsed with a rinse fluid containing surfactant, and spin dried to form a positive pattern.

The LS pattern as developed was observed under CD-SEM (CG6300, Hitachi High-Technologies Corp.) whereupon sensitivity, EL, LWR, DOF and collapse limit were evaluated by the following methods. The results are shown in Tables 9 to 12.

### [Evaluation of Sensitivity]

The optimum dose Eop (mJ/cm²) which provided a LS pattern with a line width of 18 nm and a pitch of 36 nm was determined as an index of sensitivity. A smaller value indicates a higher sensitivity.

### [Evaluation of EL]

The exposure dose which provided a LS pattern with a space width of 18 nm±10% (i.e., 16.2 to 19.8 nm) was determined. EL (%) is calculated from the exposure doses according to the following equation:

$$EL ( \% ) = ( \vert {E}_{1} - {E}_{2} \vert / Eop ) \times 100$$

wherein E₁ is an optimum exposure dose which provides a LS pattern with a line width of 16.2 nm and a pitch of 36 nm, E₂ is an optimum exposure dose which provides a LS pattern with a line width of 19.8 nm and a pitch of 36 nm, and Eop is an optimum exposure dose which provides a LS pattern with a line width of 18 nm and a pitch of 36 nm. A greater value indicates better performance.

### [Evaluation of LWR]

For the LS pattern formed by exposure at the optimum dose Eop, the line width was measured at 10 longitudinally spaced apart points, from which a 3-fold value (36) of the standard deviation (6) was determined and reported as LWR. A smaller value of 36 indicates a pattern having small roughness and uniform line width.

### [Evaluation of DOF]

As an index of DOF, a range of focus which provided a LS pattern with a size of 18 nm±10% (i.e., 16.2 to 19.8 nm) was determined. A greater value indicates a wider DOF.

### [Evaluation of Collapse Limit of Line Pattern]

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

TABLE 9

| | | Resist | PEB | | | | | Collapse |
|---|---|---|---|---|---|---|---|---|
| | | compo- | temp. | Eop | EL | LWR | DOF | limit |
| | | sition | (° C.) | (mJ/cm²) | (%) | (nm) | (nm) | (nm) |
| Example | 4-1 | R-1 | 95 | 32 | 18 | 2.3 | 120 | 10.9 |
| | 4-2 | R-2 | 100 | 32 | 19 | 2.4 | 110 | 10.7 |
| | 4-3 | R-3 | 100 | 33 | 17 | 2.5 | 120 | 10.6 |
| | 4-4 | R-4 | 95 | 34 | 18 | 2.3 | 110 | 11.1 |
| | 4-5 | R-5 | 105 | 33 | 17 | 2.4 | 100 | 11.2 |
| | 4-6 | R-6 | 100 | 32 | 17 | 2.3 | 120 | 11.3 |
| | 4-7 | R-7 | 95 | 34 | 18 | 2.5 | 110 | 11.2 |
| | 4-8 | R-8 | 95 | 33 | 19 | 2.3 | 100 | 11.3 |
| | 4-9 | R-9 | 100 | 33 | 17 | 2.4 | 110 | 11.2 |
| | 4-10 | R-10 | 100 | 32 | 18 | 2.3 | 120 | 11.3 |
| | 4-11 | R-11 | 100 | 32 | 19 | 2.4 | 120 | 11.4 |
| | 4-12 | R-12 | 95 | 33 | 17 | 2.4 | 110 | 10.8 |
| | 4-13 | R-13 | 105 | 32 | 18 | 2.5 | 120 | 10.6 |
| | 4-14 | R-14 | 100 | 32 | 17 | 2.3 | 100 | 10.9 |
| | 4-15 | R-15 | 95 | 34 | 17 | 2.4 | 110 | 10.7 |
| | 4-16 | R-16 | 95 | 35 | 18 | 2.3 | 120 | 11.1 |
| | 4-17 | R-17 | 100 | 32 | 17 | 2.5 | 110 | 11.2 |
| | 4-18 | R-18 | 95 | 32 | 18 | 2.3 | 110 | 11.4 |
| | 4-19 | R-19 | 95 | 34 | 18 | 2.4 | 120 | 11.3 |
| | 4-20 | R-20 | 100 | 33 | 17 | 2.5 | 110 | 10.9 |
| | 4-21 | R-21 | 100 | 32 | 19 | 2.4 | 110 | 10.7 |
| | 4-22 | R-22 | 100 | 33 | 17 | 2.3 | 110 | 11.2 |
| | 4-23 | R-23 | 95 | 32 | 18 | 2.4 | 110 | 11.3 |
| | 4-24 | R-24 | 95 | 32 | 18 | 2.3 | 120 | 11.2 |
| | 4-25 | R-25 | 100 | 34 | 17 | 2.5 | 110 | 11.0 |
| | 4-26 | R-26 | 100 | 33 | 19 | 2.3 | 110 | 10.7 |
| | 4-27 | R-27 | 95 | 32 | 18 | 2.3 | 120 | 11.2 |
| | 4-28 | R-28 | 100 | 33 | 17 | 2.5 | 110 | 11.0 |
| | 4-29 | R-29 | 100 | 34 | 19 | 2.5 | 110 | 10.7 |
| | 4-30 | R-30 | 95 | 32 | 18 | 2.3 | 120 | 10.9 |

TABLE 10

| | | Resist | PEB | | | | | Collapse |
|---|---|---|---|---|---|---|---|---|
| | | compo- | temp. | Eop | EL | LWR | DOF | limit |
| | | sition | (° C.) | (mJ/cm²) | (%) | (nm) | (nm) | (nm) |
| Example | 4-31 | R-31 | 100 | 32 | 19 | 2.4 | 110 | 10.7 |
| | 4-32 | R-32 | 100 | 33 | 17 | 2.5 | 120 | 10.6 |
| | 4-33 | R-33 | 95 | 34 | 18 | 2.3 | 110 | 11.1 |
| | 4-34 | R-34 | 105 | 33 | 17 | 2.4 | 100 | 11.2 |
| | 4-35 | R-35 | 100 | 32 | 17 | 2.3 | 120 | 11.3 |
| | 4-36 | R-36 | 95 | 34 | 18 | 2.5 | 110 | 11.2 |
| | 4-37 | R-37 | 95 | 33 | 19 | 2.3 | 100 | 11.3 |
| | 4-38 | R-38 | 100 | 33 | 17 | 2.4 | 110 | 11.2 |
| | 4-39 | R-39 | 100 | 32 | 18 | 2.3 | 120 | 11.3 |
| | 4-40 | R-40 | 100 | 32 | 19 | 2.4 | 120 | 11.4 |
| | 4-41 | R-41 | 95 | 33 | 17 | 2.4 | 110 | 10.8 |
| | 4-42 | R-42 | 105 | 32 | 18 | 2.5 | 120 | 10.6 |
| | 4-43 | R-43 | 100 | 32 | 17 | 2.3 | 100 | 10.9 |
| | 4-44 | R-44 | 95 | 34 | 17 | 2.4 | 110 | 10.7 |
| | 4-45 | R-45 | 95 | 35 | 18 | 2.3 | 120 | 11.1 |
| | 4-46 | R-46 | 100 | 32 | 17 | 2.5 | 110 | 11.2 |
| | 4-47 | R-47 | 95 | 32 | 18 | 2.3 | 110 | 11.4 |
| | 4-48 | R-48 | 95 | 34 | 18 | 2.4 | 120 | 11.3 |
| | 4-49 | R-49 | 100 | 33 | 17 | 2.5 | 110 | 10.9 |
| | 4-50 | R-50 | 100 | 32 | 19 | 2.4 | 110 | 10.7 |
| | 4-51 | R-51 | 100 | 33 | 17 | 2.3 | 110 | 11.2 |
| | 4-52 | R-52 | 95 | 32 | 18 | 2.4 | 110 | 11.3 |
| | 4-53 | R-53 | 95 | 32 | 18 | 2.3 | 120 | 11.2 |
| | 4-54 | R-54 | 100 | 34 | 17 | 2.5 | 110 | 11.0 |
| | 4-55 | R-55 | 100 | 33 | 19 | 2.3 | 110 | 10.7 |
| | 4-56 | R-56 | 95 | 32 | 18 | 2.3 | 120 | 11.2 |
| | 4-57 | R-57 | 100 | 33 | 17 | 2.5 | 110 | 11.0 |
| | 4-58 | R-58 | 100 | 34 | 19 | 2.5 | 110 | 10.7 |
| | 4-59 | R-59 | 100 | 33 | 17 | 2.5 | 110 | 11.0 |
| | 4-60 | R-60 | 100 | 34 | 19 | 2.5 | 110 | 10.7 |

TABLE 11

| | | Resist | PEB | | | | | Collapse |
|---|---|---|---|---|---|---|---|---|
| | | compo- | temp. | Eop | EL | LWR | DOF | limit |
| | | sition | (° C.) | (mJ/cm²) | (%) | (nm) | (nm) | (nm) |
| Compar- | 4-1 | CR-1 | 95 | 37 | 14 | 3.1 | 90 | 12.8 |
| ative | 4-2 | CR-2 | 100 | 38 | 13 | 2.8 | 80 | 12.7 |
| Example | 4-3 | CR-3 | 100 | 36 | 14 | 2.7 | 80 | 13.1 |
| | 4-4 | CR-4 | 100 | 38 | 13 | 2.9 | 90 | 12.7 |
| | 4-5 | CR-5 | 95 | 34 | 15 | 3.3 | 60 | 14.1 |
| | 4-6 | CR-6 | 100 | 37 | 13 | 2.8 | 60 | 12.9 |
| | 4-7 | CR-7 | 100 | 37 | 15 | 2.7 | 90 | 11.9 |
| | 4-8 | CR-8 | 100 | 38 | 13 | 2.8 | 80 | 12.1 |
| | 4-9 | CR-9 | 95 | 36 | 14 | 2.7 | 90 | 12.2 |
| | 4-10 | CR-10 | 100 | 37 | 13 | 2.8 | 80 | 12.4 |
| | 4-11 | CR-11 | 100 | 36 | 15 | 2.9 | 80 | 12.1 |
| | 4-12 | CR-12 | 100 | 36 | 14 | 2.9 | 80 | 11.8 |
| | 4-13 | CR-13 | 95 | 37 | 13 | 2.8 | 90 | 11.9 |
| | 4-14 | CR-14 | 105 | 37 | 11 | 2.7 | 50 | 12.9 |
| | 4-15 | CR-15 | 100 | 38 | 15 | 2.9 | 70 | 12.5 |
| | 4-16 | CR-16 | 95 | 36 | 13 | 2.8 | 80 | 12.3 |
| | 4-17 | CR-17 | 95 | 33 | 14 | 3.3 | 60 | 14.2 |
| | 4-18 | CR-18 | 100 | 39 | 13 | 2.8 | 60 | 12.4 |
| | 4-19 | CR-19 | 100 | 37 | 14 | 2.9 | 60 | 12.4 |
| | 4-20 | CR-20 | 95 | 36 | 15 | 2.8 | 90 | 11.9 |
| | 4-21 | CR-21 | 100 | 36 | 14 | 2.7 | 80 | 12.1 |
| | 4-22 | CR-22 | 100 | 33 | 11 | 3.3 | 90 | 14.3 |
| | 4-23 | CR-23 | 100 | 36 | 13 | 2.7 | 60 | 12.4 |
| | 4-24 | CR-24 | 95 | 38 | 14 | 2.9 | 80 | 12.1 |
| | 4-25 | CR-25 | 100 | 37 | 15 | 2.8 | 80 | 11.8 |
| | 4-26 | CR-26 | 100 | 37 | 14 | 2.7 | 90 | 12.9 |
| | 4-27 | CR-27 | 100 | 36 | 14 | 2.8 | 90 | 12.5 |
| | 4-28 | CR-28 | 95 | 37 | 15 | 3 | 80 | 12.3 |
| | 4-29 | CR-29 | 100 | 37 | 14 | 3.1 | 60 | 12.6 |
| | 4-30 | CR-30 | 100 | 38 | 13 | 3.1 | 80 | 14.2 |

TABLE 12

| | | Resist | PEB | | | | | Collapse |
|---|---|---|---|---|---|---|---|---|
| | | compo- | temp. | Eop | EL | LWR | DOF | limit |
| | | sition | (° C.) | (mJ/cm²) | (%) | (nm) | (nm) | (nm) |
| Compar- | 4-31 | CR-31 | 100 | 36 | 14 | 2.8 | 80 | 12.4 |
| ative | 4-32 | CR-32 | 100 | 38 | 13 | 2.7 | 90 | 12.4 |
| Example | 4-33 | CR-33 | 95 | 34 | 14 | 2.9 | 60 | 11.9 |
| | 4-34 | CR-34 | 95 | 37 | 13 | 2.9 | 60 | 12.1 |
| | 4-35 | CR-35 | 100 | 37 | 15 | 2.8 | 90 | 11.8 |
| | 4-36 | CR-36 | 100 | 38 | 13 | 3.3 | 80 | 11.9 |
| | 4-37 | CR-37 | 95 | 36 | 14 | 2.8 | 90 | 12.9 |
| | 4-38 | CR-38 | 100 | 37 | 13 | 2.9 | 80 | 12.5 |
| | 4-39 | CR-39 | 100 | 38 | 15 | 2.8 | 80 | 12.3 |
| | 4-40 | CR-40 | 95 | 37 | 14 | 2.7 | 80 | 14.2 |
| | 4-41 | CR-41 | 95 | 37 | 14 | 3.3 | 90 | 12.4 |
| | 4-42 | CR-42 | 100 | 36 | 11 | 2.7 | 50 | 12.4 |
| | 4-43 | CR-43 | 100 | 37 | 13 | 2.9 | 80 | 11.9 |
| | 4-44 | CR-44 | 100 | 37 | 14 | 2.8 | 90 | 12.1 |
| | 4-45 | CR-45 | 100 | 38 | 15 | 3 | 60 | 14.3 |
| | 4-46 | CR-46 | 95 | 38 | 14 | 3.1 | 80 | 12.4 |
| | 4-47 | CR-47 | 95 | 36 | 14 | 3.1 | 80 | 12.1 |
| | 4-48 | CR-48 | 100 | 37 | 15 | 2.8 | 90 | 11.8 |
| | 4-49 | CR-49 | 100 | 38 | 14 | 2.7 | 90 | 12.9 |
| | 4-50 | CR-50 | 95 | 37 | 13 | 3.3 | 80 | 12.5 |
| | 4-51 | CR-51 | 100 | 37 | 14 | 2.7 | 90 | 12.3 |
| | 4-52 | CR-52 | 100 | 36 | 13 | 2.6 | 50 | 12.6 |

It is demonstrated in Tables 9 to 12 that chemically amplified resist compositions comprising PAGs within the scope of the invention exhibit a high sensitivity and improved values of EL, LWR and DOF. Small values of collapse limit attest that in forming a small-size pattern, the pattern is resistant to collapse. The resist compositions are useful in the EUV lithography process.

### [5] EUV Lithography Test 2

### Examples 5-1 to 5-60 and Comparative Examples 5-1 to 5-52

Each of the chemically amplified resist compositions (R-1 to R-60, CR-1 to CR-52) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 (ASML, NA 0.33, σ 0.9/0.6, quadrupole illumination), the resist film was exposed to EUV through a mask bearing a hole pattern having a pitch of 46 nm+20% bias (on-wafer size). The resist film was baked (PEB) on a hotplate at the temperature shown in Tables 13 to 16 for 60 seconds and developed in a 2.38 wt % TMAH aqueous solution for 30 seconds to form a hole pattern having a size of 23 nm.

The pattern as developed was observed under CD-SEM (CG6300, Hitachi High-Technologies Corp.). The dose (mJ/cm²) at which a pattern with a hole size of 23 nm was printed was determined as an index of sensitivity. The size of 50 holes was measured, from which a 3-fold value (3σ) of the standard deviation (σ) was determined as a dimensional variation (or CDU). The results are shown in Tables 13 to 16.

TABLE 13

| | | Resist | PEB temp. | Optimum dose | CDU |
|---|---|---|---|---|---|
| | | composition | (° C.) | (mJ/cm²) | (nm) |
| Example | 5-1 | R-1 | 95 | 22 | 2.2 |
| | 5-2 | R-2 | 95 | 23 | 2.4 |
| | 5-3 | R-3 | 90 | 23 | 2.3 |
| | 5-4 | R-4 | 90 | 23 | 2.3 |
| | 5-5 | R-5 | 90 | 22 | 2.4 |
| | 5-6 | R-6 | 95 | 24 | 2.5 |
| | 5-7 | R-7 | 95 | 23 | 2.4 |
| | 5-8 | R-8 | 90 | 24 | 2.3 |
| | 5-9 | R-9 | 95 | 24 | 2.3 |
| | 5-10 | R-10 | 95 | 23 | 2.4 |
| | 5-11 | R-11 | 95 | 25 | 2.4 |
| | 5-12 | R-12 | 90 | 22 | 2.3 |
| | 5-13 | R-13 | 90 | 24 | 2.3 |
| | 5-14 | R-14 | 90 | 23 | 2.4 |
| | 5-15 | R-15 | 90 | 23 | 2.3 |
| | 5-16 | R-16 | 85 | 24 | 2.3 |
| | 5-17 | R-17 | 95 | 25 | 2.4 |
| | 5-18 | R-18 | 95 | 24 | 2.2 |
| | 5-19 | R-19 | 90 | 22 | 2.5 |
| | 5-20 | R-20 | 95 | 23 | 2.4 |
| | 5-21 | R-21 | 95 | 23 | 2.4 |
| | 5-22 | R-22 | 95 | 24 | 2.3 |
| | 5-23 | R-23 | 95 | 24 | 2.3 |
| | 5-24 | R-24 | 90 | 25 | 2.4 |
| | 5-25 | R-25 | 95 | 22 | 2.3 |
| | 5-26 | R-26 | 95 | 23 | 2.2 |
| | 5-27 | R-27 | 90 | 25 | 2.5 |
| | 5-28 | R-28 | 95 | 22 | 2.2 |
| | 5-29 | R-29 | 95 | 23 | 2.4 |
| | 5-30 | R-30 | 95 | 22 | 2.3 |

TABLE 14

| | | Resist | PEB temp. | Optimum dose | CDU |
|---|---|---|---|---|---|
| | | composition | (° C.) | (mJ/cm²) | (nm) |
| Example | 5-31 | R-31 | 95 | 23 | 2.3 |
| | 5-32 | R-32 | 90 | 23 | 2.3 |
| | 5-33 | R-33 | 90 | 23 | 2.3 |
| | 5-34 | R-34 | 90 | 22 | 2.4 |
| | 5-35 | R-35 | 95 | 24 | 2.5 |
| | 5-36 | R-36 | 95 | 23 | 2.3 |
| | 5-37 | R-37 | 90 | 24 | 2.2 |
| | 5-38 | R-38 | 95 | 24 | 2.5 |
| | 5-39 | R-39 | 95 | 23 | 2.3 |
| | 5-40 | R-40 | 95 | 25 | 2.2 |
| | 5-41 | R-41 | 90 | 22 | 2.2 |
| | 5-42 | R-42 | 90 | 24 | 2.4 |
| | 5-43 | R-43 | 90 | 23 | 2.3 |
| | 5-44 | R-44 | 90 | 23 | 2.3 |
| | 5-45 | R-45 | 85 | 24 | 2.3 |
| | 5-46 | R-46 | 95 | 25 | 2.3 |
| | 5-47 | R-47 | 95 | 24 | 2.4 |
| | 5-48 | R-48 | 90 | 22 | 2.5 |
| | 5-49 | R-49 | 95 | 23 | 2.4 |
| | 5-50 | R-50 | 95 | 23 | 2.3 |
| | 5-51 | R-51 | 95 | 24 | 2.3 |
| | 5-52 | R-52 | 95 | 24 | 2.3 |
| | 5-53 | R-53 | 90 | 25 | 2.3 |
| | 5-54 | R-54 | 95 | 22 | 2.2 |
| | 5-55 | R-55 | 95 | 23 | 2.5 |
| | 5-56 | R-56 | 90 | 25 | 2.3 |
| | 5-57 | R-57 | 95 | 22 | 2.3 |
| | 5-58 | R-58 | 95 | 23 | 2.2 |
| | 5-59 | R-59 | 95 | 22 | 2.5 |
| | 5-60 | R-60 | 95 | 23 | 2.3 |

TABLE 15

| | | Resist | PEB temp. | Optimum dose | CDU |
|---|---|---|---|---|---|
| | | composition | (° C.) | (mJ/cm²) | (nm) |
| Comparative | 5-1 | CR-1 | 95 | 29 | 2.9 |
| Example | 5-2 | CR-2 | 95 | 28 | 2.8 |
| | 5-3 | CR-3 | 95 | 27 | 2.7 |
| | 5-4 | CR-4 | 90 | 27 | 2.8 |
| | 5-5 | CR-5 | 90 | 24 | 3.4 |
| | 5-6 | CR-6 | 95 | 28 | 2.8 |
| | 5-7 | CR-7 | 90 | 29 | 2.7 |
| | 5-8 | CR-8 | 90 | 27 | 2.9 |
| | 5-9 | CR-9 | 90 | 28 | 2.7 |
| | 5-10 | CR-10 | 95 | 28 | 2.8 |
| | 5-11 | CR-11 | 95 | 28 | 2.6 |
| | 5-12 | CR-12 | 95 | 29 | 2.9 |
| | 5-13 | CR-13 | 85 | 27 | 2.8 |
| | 5-14 | CR-14 | 95 | 29 | 2.8 |
| | 5-15 | CR-15 | 95 | 27 | 2.7 |
| | 5-16 | CR-16 | 90 | 27 | 2.8 |
| | 5-17 | CR-17 | 95 | 24 | 3.3 |
| | 5-18 | CR-18 | 95 | 28 | 2.9 |
| | 5-19 | CR-19 | 90 | 29 | 2.7 |
| | 5-20 | CR-20 | 90 | 28 | 2.8 |
| | 5-21 | CR-21 | 95 | 27 | 2.7 |
| | 5-22 | CR-22 | 90 | 24 | 3.3 |
| | 5-23 | CR-23 | 90 | 29 | 2.8 |
| | 5-24 | CR-24 | 90 | 28 | 2.7 |
| | 5-25 | CR-25 | 95 | 29 | 2.9 |
| | 5-26 | CR-26 | 95 | 27 | 2.8 |
| | 5-27 | CR-27 | 95 | 28 | 2.8 |
| | 5-28 | CR-28 | 90 | 27 | 2.9 |
| | 5-29 | CR-29 | 95 | 28 | 2.8 |
| | 5-30 | CR-30 | 95 | 29 | 2.8 |

TABLE 16

| | | Resist | PEB temp. | Optimum dose | CDU |
|---|---|---|---|---|---|
| | | composition | (° C.) | (mJ/cm²) | (nm) |
| Comparative | 5-31 | CR-31 | 95 | 27 | 3.4 |
| Example | 5-32 | CR-32 | 90 | 28 | 2.8 |
| | 5-33 | CR-33 | 95 | 27 | 2.7 |
| | 5-34 | CR-34 | 95 | 27 | 2.9 |
| | 5-35 | CR-35 | 95 | 24 | 2.7 |
| | 5-36 | CR-36 | 90 | 28 | 2.9 |
| | 5-37 | CR-37 | 90 | 29 | 2.8 |
| | 5-38 | CR-38 | 90 | 27 | 2.8 |
| | 5-39 | CR-39 | 90 | 27 | 2.6 |
| | 5-40 | CR-40 | 85 | 28 | 2.9 |
| | 5-41 | CR-41 | 95 | 28 | 2.8 |
| | 5-42 | CR-42 | 95 | 27 | 2.8 |
| | 5-43 | CR-43 | 90 | 27 | 2.6 |
| | 5-44 | CR-44 | 95 | 24 | 2.9 |
| | 5-45 | CR-45 | 95 | 28 | 2.8 |
| | 5-46 | CR-46 | 90 | 27 | 2.8 |
| | 5-47 | CR-47 | 90 | 28 | 2.7 |
| | 5-48 | CR-48 | 95 | 27 | 2.9 |
| | 5-49 | CR-49 | 95 | 27 | 2.8 |
| | 5-50 | CR-50 | 95 | 24 | 2.8 |
| | 5-51 | CR-51 | 90 | 28 | 2.9 |
| | 5-52 | CR-52 | 95 | 27 | 2.8 |

It is demonstrated in Tables 13 to 16 that chemically amplified resist compositions within the scope of the invention exhibit a high sensitivity and satisfactory CDU.

Japanese Patent Application No. 2024-227503 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A monomer having the formula (A): 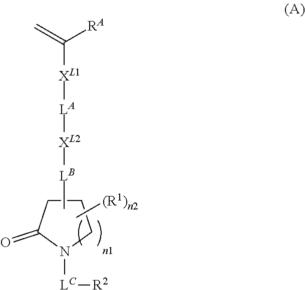
wherein n1 is 1, 2 or 3, n2 is 0, 1 or 2,
R^{A} is hydrogen, fluorine, methyl or trifluoromethyl,
X^{L1} is a single bond, optionally substituted phenylene group or optionally substituted naphthylene group,
X^{L2} is a single bond or C₁-C₄₀ hydrocarbylene group which may contain a heteroatom,
L^{A} and L^{B} are each independently a single bond, ether bond, ester bond, amide bond, carbonate bond or carbamate bond,
L^{C} is a single bond, carbonyl group or ester bond,
R¹ is halogen, nitro, cyano, hydroxy, carboxy, or a C₁-C₂₀ hydrocarbyl group, C₁-C₂₀ hydrocarbyloxy group or C₁-C₂₀ hydrocarbylthio group, which may contain a heteroatom; when n2=2, two R¹ may be identical or different, and
R² is a C₁-C₂₀ hydrocarbyl group which may contain a heteroatom.

2. The monomer of claim 1, having the formula (A1): 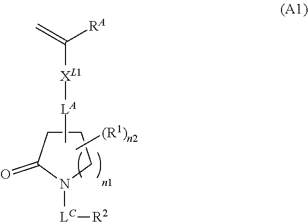
wherein n1, n2, R^{A}, X^{L1}, L^{A}, L^{C}, R¹ and R² are as defined above.

3. A polymer comprising repeat units derived from the monomer of claim 1.

4. The polymer of claim 3, further comprising repeat units having the formula (a1) or (a2): 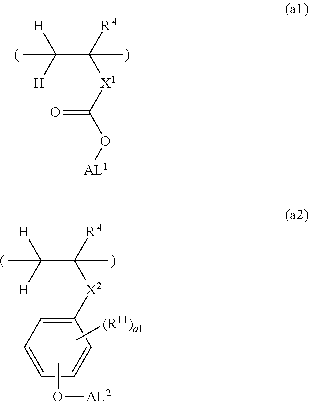
wherein R^{A} is each independently hydrogen, fluorine, methyl or trifluoromethyl,
X¹ is a single bond, phenylene, naphthylene, *—C(═O)—O—X¹¹— or *—C(═O)—NH—X¹¹—, the phenylene or naphthylene group may be substituted with hydroxy, nitro, cyano, optionally fluorinated C₁-C₁₀ saturated hydrocarbyl moiety, optionally fluorinated C₁-C₁₀ saturated hydrocarbyloxy moiety, or halogen, X¹¹ is a C₁-C₁₀ saturated hydrocarbylene group, phenylene group or naphthylene group, the saturated hydrocarbylene group may contain hydroxy, ether bond, ester bond or lactone ring,
X² is a single bond, *—C(═O)—O— or *—C(═O)—NH—,
* designates a point of attachment to the carbon atom in the backbone,
R¹¹ is halogen, cyano, hydroxy, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom; when a1 is 2, 3 or 4, a plurality of R¹¹ may be identical or different,
AL¹ and AL² are each independently an acid labile group, and
a1 is 0, 1, 2, 3 or 4.

5. The polymer of claim 3, further comprising repeat units having the formula (a3): 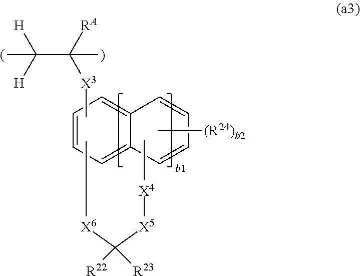
wherein b1 is 0 or 1, b2 is 0, 1, 2 or 3 when b1=0, b2 is 0, 1, 2, 3, 4 or 5 when b1=1,
R^{A} is hydrogen, fluorine, methyl or trifluoromethyl,
X³ is a single bond, *—C(═O)—O— or *—C(═O)—NH—, * designates a point of attachment to the carbon atom in the backbone,
X⁴ is a single bond, C₁-C₄ aliphatic hydrocarbylene group, carbonyl, sulfonyl or a group obtained by combining the foregoing,
X⁵ and X⁶ are each independently oxygen or sulfur, X⁴ and X⁶ are attached to adjoining carbon atoms on the aromatic ring,
R¹² and R¹³ are each independently hydrogen or a C₁-C₂₀ hydrocarbyl group which may contain a heteroatom, R¹² and R¹³ may bond together to form a ring with the carbon atom to which they are attached,
R¹⁴ is halogen, hydroxy, cyano, nitro, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{14A})(R^{14B}), R^{14A} and R^{14B} are each independently hydrogen or a C₁-C₆ hydrocarbyl group; and when b2 is 2 or more, a plurality of R¹⁴ may be identical or different and a plurality of R¹⁴ may bond together to form a ring with the carbon atom on the aromatic ring to which they are attached.

6. The polymer of claim 3, further comprising repeat units having the formula (b1) or (b2): 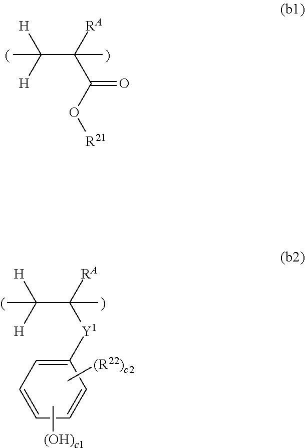
wherein R^{A} is hydrogen, fluorine, methyl or trifluoromethyl,
Y¹ is a single bond or *—C(═O)—O—, * designates a point of attachment to the carbon atom in the backbone,
R²¹ is hydrogen or a C₁-C₂₀ group containing at least one structure selected from hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—),
R²² is halogen, carboxy, nitro, cyano, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom; when c2 is 2, 3 or 4, a plurality of R²² may be identical or different,
c1 is 1, 2, 3 or 4, c2 is 0, 1, 2, 3 or 4, and 1≤c1+c2≤5.

7. The polymer of claim 3, further comprising repeat units of at least one type selected from repeat units having the formula (c1), repeat units having the formula (c2), repeat units having the formula (c3), repeat units having the formula (c4), and repeat units having the formula (c5): 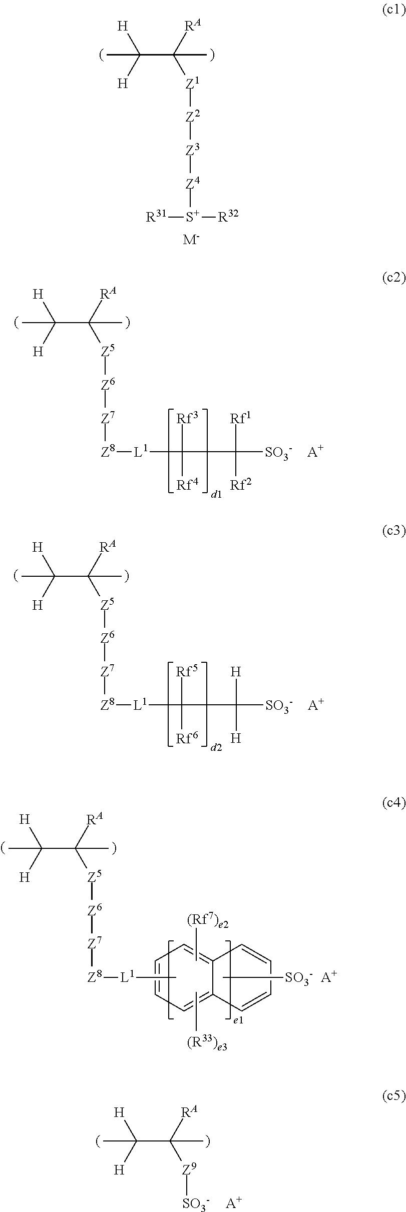
wherein d1 and d2 are each independently 0, 1, 2 or 3,
e1 is 0 or 1, e2 is 0, 1, 2, 3 or 4, e3 is 0, 1, 2, 3 or 4, meeting 0≤e2+e3≤4 when e1=0, and 0≤e2+e3≤6 when e1=1,
R^{A} is each independently hydrogen, fluorine, methyl or trifluoromethyl,
Z¹ is a single bond or optionally substituted phenylene group,
Z² is a single bond, **—C(═O)—O—Z²¹—, **—C(═O)—NH—Z²¹—, or **—O—Z²¹—, Z²¹ is a C₁-C₆ aliphatic hydrocarbylene group, phenylene group or a divalent group obtained by combining the foregoing, which may contain halogen, carbonyl moiety, ester bond, ether bond or hydroxy moiety,
Z³ is a single bond, ether bond, ester bond, sulfonate ester bond, amide bond, sulfonamide bond, carbonate bond or carbamate bond,
Z⁴ is a single bond or a C₁-C₆ aliphatic hydrocarbylene group, phenylene group or a divalent group obtained by combining the foregoing, which may contain halogen, carbonyl moiety, ester bond, ether bond or hydroxy moiety,
Z⁵ is each independently a single bond, optionally substituted phenylene group, naphthylene group or *—C(═O)—O—Z⁵¹—, Z⁵¹ is a C₁-C₁₀ aliphatic hydrocarbylene group, phenylene group or naphthylene group, the aliphatic hydrocarbylene group may contain halogen, hydroxy, ether bond, ester bond or lactone ring,
Z⁶ is a single bond, ether bond, ester bond, sulfonate ester bond, amide bond, sulfonamide bond, carbonate bond or carbamate bond,
Z⁷ is each independently a single bond, ***—Z⁷—C(═O)—O—, ***—C(═O)—NH—Z⁷— or ***—O—Z⁷¹—, Z⁷¹ is a C₁-C₂₀ hydrocarbylene group which may contain a heteroatom,
Z⁸ is each independently a single bond, ****—Z⁸—C(═O)—O—, ****—C(═O)—NH—Z⁸¹— or ****—O—Z⁸¹—, Z⁸¹ is a C₁-C₂₀ hydrocarbylene group which may contain a heteroatom,
Z⁹ is a single bond, methylene, ethylene, phenylene, fluorinated phenylene, trifluoromethyl-substituted phenylene, *—C(═O)—O—Z⁹¹—, *—C(═O)—N(H)—Z⁹¹— or *—O—Z⁹¹—, Z⁹¹ is a C₁-C₆ aliphatic hydrocarbylene group, phenylene group, fluorinated phenylene group, or trifluoromethyl-substituted phenylene group, which may contain carbonyl, ester bond, ether bond or hydroxy,
* designates a point of attachment to the carbon atom in the backbone, ** designates a point of attachment to Z¹, *** designates a point of attachment to Z⁶, **** designates a point of attachment to Z⁷,
L¹ is a single bond, ether bond, ester bond, carbonyl group, sulfonate ester bond, sulfonamide bond, carbonate bond or carbamate bond,
Rf¹ and Rf² are each independently fluorine or a C₁-C₆ fluorinated saturated hydrocarbyl group,
Rf³ and Rf⁴ are each independently hydrogen, fluorine or a C₁-C₆ fluorinated saturated hydrocarbyl group,
Rf⁵ and Rf⁶ are each independently hydrogen, fluorine or a C₁-C₆ fluorinated saturated hydrocarbyl group, excluding that all Rf⁵ and Rf⁶ are hydrogen at the same time,
Rf⁷ is fluorine, a C₁-C₆ fluorinated alkyl group, C₁-C₆ fluorinated alkoxy group, or C₁-C₆ fluorinated alkylthio group; when e2 is 2, 3 or 4, a plurality of Rf may be identical or different,
R³¹ and R³² are each independently a C₁-C₂₀ hydrocarbyl group which may contain a heteroatom, R³¹ and R³² may bond together to form a ring with the sulfur atom to which they are attached,
R³³ is halogen exclusive of fluorine, or a C₁-C₂₀ hydrocarbyl group which may contain a heteroatom; when e3 is 2, 3 or 4, a plurality of R³³ may be identical or different and a plurality of R³³ may bond together to form a ring with the carbon atoms to which they are attached,
M⁻ is a non-nucleophilic counter ion, and
A⁺ is an onium cation.

8. A chemically amplified resist composition comprising a base polymer containing the polymer of claim 3.

9. The resist composition of claim 8, further comprising an organic solvent.

10. The resist composition of claim 8, further comprising a quencher.

11. The resist composition of claim 8, further comprising an acid generator.

12. The resist composition of claim 8, further comprising a surfactant.

13. A pattern forming process comprising the steps of applying the chemically amplified resist composition of claim 8 onto a substrate to form a resist film thereon, exposing the resist film to high-energy radiation, and developing the exposed resist film in a developer.

14. The process of claim 13 wherein the high-energy radiation is KrF excimer laser, ArF excimer laser, EB or EUV of wavelength 3 to 15 nm.
